(19) United States
(12) Patent Application Publication
(10) Pub. No.: US 2003/0018166 A1
(43) Pub. Date: Jan. 23, 2003

(54) Structure of isocitrate lyase enzyme from mycobacterium tuberculosis and inhibitory agents to combat persistent infection

(76) Inventors: James C. Sacchettini, College Station, TX (US); William R. Jacobs JR., Bronx, NY (US); David G. Russell, Ithaca, NY (US); John D. McKinney, New York, NY (US); Vivek Sharma, Pearland, TX (US); Sujata Sharma, Pearland, TX (US); Kerstin A. Honer Zu Bentrup, New Orleans, LA (US)

Correspondence Address: Scott Reese, Ph.D., Howrey, Simon, Arnold & White, L.L.P., 750 Bering Drive, Houston, TX, 77057-2198, US

(21) Appl. No.: 09/921,635

(22) Filed: Aug. 3, 2001

Related U.S. Application Data
(60) Provisional application No. 60/222,768, filed on Aug. 3, 2000.

(51) Int. Cl. C07K 1/00; C07K 14/00; C07K 17/00

(52) U.S. Cl. 530/350

(57) ABSTRACT

The invention provides methods and compositions for use in identifying inhibitors of biochemical pathways important for persistent infection, allowing the identification and/or design of improved therapeutics for treating persistent infections by pathogenic microbes. Particularly disclosed is the importance of the glyoxylate shunt to the persistent phase of various infectious agents, including Mycobacteria, such as M. tuberculosis, and the identification of preferred targets for drug development, including the enzymes isocitrate lyase (ICL) and malate synthase. Crystals and three-dimensional structures of M. tuberculosis ICL, without ligand and in complex with two inhibitors are also disclosed, for exemplary use in the design of inhibitors and therapeutic agents.

### BACKGROUND OF THE INVENTION

The present application claims priority to co-pending U.S. provisional patent application Ser. No. 60/222,768, filed Aug. 03, 2000, the entire text and figures of which application are specifically incorporated by reference herein without disclaimer. The U.S. Government owns rights in the present invention pursuant to Grant Numbers AI 43268 and GM 62410 from the National Institutes of Health.

### FIELD OF THE INVENTION

The present invention generally relates to the fields of pathogenic microbes and to therapeutic agents for treating persistent infections, including infection by M. tuberculosis. Through rigorous definition of an important pathway for persistent infection, the invention provides preferred targets for drug development from the glyoxylate shunt pathway, such as the isocitrate lyase and malate synthase enzymes. Exemplary embodiments of the invention concern crystals and three-dimensional structures of M. tuberculosis isocitrate lyase in complex with inhibitors, for particular use in the design of inhibitors and therapeutic agents.

### DESCRIPTION OF RELATED ART

Although modem medicine has provided many weapons to combat disease, infection by pathogenic microbes still poses a significant threat to human life. In recent times, an increasing number of microbes have developed resistance to many commonly used antimicrobial agents, thereby contributing to a new spread of disease. Mycobacteria are examples of microbial pathogens that exhibit persistent infection. Mycobacterium tuberculosis, the causative agent of the tuberculosis (TB) disease, exhibits a penetrance in the human population that is rivaled by few other pathogens. Tuberculosis remains the largest cause of death in the world from a single infectious disease and causes many fatalities in developing countries.

The success of M. tuberculosis is dependent on its ability to persist and maintain chronic infection in humans (Parrish et al., 1998). During chronic tuberculosis, the bacteria exist in diverse metabolic states that are not targeted by conventional antimycobacterials (Mitchison, 1980). Lengthy regimens of anti-TB drugs are necessary and are currently the only way to even approach killing of the persistent bacteria.

The current drugs have further drawbacks, such as targeting only a small number of bacterial processes, notably cell wall formation and chromosomal replication (Parrish et al., 1998; McKinney et al. 1998). The effectiveness of drugs aimed at intervening in such processes is further limited by the ability of the organisms to adapt under the selective pressure of the treatment and become resistant.

The emergence of drug resistant strains is a constant threat to the use of the currently available antimycobacterial agents. The development of multiple drug resistant strains of M. tuberculosis has resulted in fatal outbreaks of disease, including those in the United States. In fact, resistance to isoniazid, one of the most widely used anti-tuberculosis drugs for both therapy and prophylaxis, is now quite common. In recent years, resistance to isoniazid has been as high as 26% in some areas of the United States.

Despite ongoing efforts in this field, new drugs are thus urgently needed for use against TB and other microbes involved in persistent infection. As these pathogens are often able to evade currently available antimycobacterials, there is a particular need to identify better targets for drug development, leading to the identification or design of long sought after therapeutics. The development of agents particularly attuned to combating the persistent stage of infection would represent a significant advance in the art.

### SUMMARY OF THE INVENTION

The present invention satisfies these needs in the art by providing important targets for use in the development of improved antimicrobials, particularly in the discovery and/or design of therapeutic agents to counteract the persistent stage of infectious pathogens. The invention is based, in part, upon the rigorous definition of pathways important for persistent infection, particularly the glyoxylate shunt pathway, and on the identification of the isocitrate lyase (ICL) and malate synthase enzymes as important targets for inhibition. In certain embodiments, the invention particularly provides crystallographic and three-dimensional structural information for M. tuberculosis isocitrate lyase, without ligand and in complex with two inhibitors, for exemplary use in the design of inhibitors and therapeutic agents.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “inhibitor”, for example, as used herein, means “at least a first inhibitor”. The operable limits and parameters of combinations, as with the amounts of any single agent, will be known to those of ordinary skill in the art in light of the present disclosure.

In overall aspects, the present invention provides methods for identifying agents for use in treating or preventing persistent microbial infections. Such methods generally comprise first choosing or selecting an enzyme or “enzyme target” from, or closely functionally associated with, the glyoxylate shunt pathway of a pathogenic microbe that exhibits a persistent stage of infection. Next, the methods generally comprise identifying, selecting or designing a compound or agent that inhibits the chosen or selected enzyme target, thereby identifying and enabling the production of a compound, inhibitor or “agent” for use in treating or preventing a persistent microbial infection.

Currently preferred selected enzyme targets are isocitrate lyase and malate synthase, unique enzymes of the glyoxylate shunt. Other suitable selected enzyme targets are the malate dehydrogenase, citrate synthase and aconitase isoenzymes of the glyoxylate shunt. Further candidates as selected enzyme targets are acetyl CoA synthase, fructose-1,6-bisphosphatase and other acetyltransferases and transporters functionally related to the effective operation of the glyoxylate shunt.

These methods may utilize selected enzyme targets from or associated with the glyoxylate shunt pathway in any one of a variety of microbial pathogens, particularly intracellular microbial pathogens, such as those that cause persistent infections, most particularly intracellular microbial pathogens that participate in persistent infections in inflammatory macrophages in vivo.

Irrespective of the source of the selected enzyme targets and/or particular pathogen employed in the screening methods, due to the unifying nature of the glyoxylate shunt pathway across the spectrum of intracellular microbial pathogens, the compounds, inhibitors and agents identified by these methods will have broad spectrum activity across this class of microbes. For example, although a mycobacterial isolated enzyme or survival assay may be chosen, the compounds, inhibitors and agents identified will not be limited to uses against mycobacteria, but may be used against any pathogen target of the invention, such as fungi. However, should the intent be to particularly develop agents for use against a given pathogen, e.g., M. tuberculosis, it is evidently an advantage of the invention that selected enzyme targets from M. tuberculosis may be employed in these methods.

Thus, the selected enzyme targets may be enzymes isolated from or flnctional within mycobacteria, such as M. tuberculosis or M. avium; from pathogenic fungi, such as C. albicans; and from other organisms, such as Pseudomonas, Salmonella, Yersinia, and Leishmania, each of which cause persistent infection in animals and humans.

Aside from the source of the selected enzyme target or original microbial pathogen used in the screening methods, the compounds, inhibitors and agents so identified may inhibit the same or the counterpart selected enzyme target from a mycobacterium, thereby identifying an agent for use in treating or preventing a persistent mycobacterial infection. Wherein, the compounds, inhibitors and agents so identified inhibit the same or the counterpart selected enzyme target from M. tuberculosis, such compounds, inhibitors and agents are effective for use in treating or preventing persistent or chronic tuberculosis. Irrespective of the source of the selected enzyme target or original microbial pathogen, the compounds, inhibitors and agents so identified may inhibit the same or the counterpart selected enzyme target from a fungus, thereby identifying an agent for use in treating or preventing a persistent fungal infection.

One of the advantageous insights of the present invention is that the selected enzyme target, whether isolated or obtained from, or maintained present within the intact host, should be a selected enzyme target from an intracellular microbial pathogen grown on a carbon source in vitro that mimics the nutrient environment encountered during the persistent phase of infection in vivo. Accordingly, the screening or other means of inhibitor identification will facilitate the identification of compounds, inhibitors and agents will therapeutic or prophylactic utility. In particular, the methods will favor the identification of compounds, inhibitors and agents that are effective against treating or preventing the persistent phase of infection, thus satisfying the most urgent need in the art.

Although preferably obtained from a microbial pathogen grown on a carbon source in vitro that mimics the nutrient environment encountered during the persistent phase of infection in vivo, compounds that inhibit the selected enzyme target may be either pre-selected or identified by testing the ability of candidate compounds to inhibit the activity of the selected enzyme target in a cell-free enzyme activity assay.

In other methods, compounds that inhibit the selected enzyme target may be identified more directly by testing the ability of candidate compounds to inhibit the growth of the intracellular microbial pathogen when grown on a carbon source in vitro that mimics the nutrient environment encountered during the persistent phase of infection in vivo. When such methods are used, the candidate compound may have been pre-screened using the foregoing type of isolated enzyme assay.

The “growth on a carbon source in vitro that mimics the nutrient environment encountered during the persistent phase of infection in vivo” means that the rich media, typically employed without concern in the prior art, should be avoided. Exemplary carbon sources for use in vitro that mimic the nutrient environment of the persistent phase in vivo infection are C₂ carbon sources, such as acetate.

Other preferred embodiments of this invention are those aspects that concern identifying compounds and inhibitors by testing the ability of candidate compounds to differentially inhibit the growth of an intracellular microbial pathogen when grown in vitro on two different carbon sources, a first carbon source that mimics the nutrient environment encountered during the persistent phase of infection in vivo, i.e., that induces the glyoxylate shunt, and a second carbon source that renders the glyoxylate shunt dispensable. For example, the first carbon source will be a C₂ carbon source, such as acetate, and the second carbon source will be a C₆ carbon source, such as glucose.

Under such conditions, the preferred candidate inhibitors are those that exhibit “differential inhibitory properties”. That is, those that have the ability to preferentially inhibit microbial growth or survival on a C₂ carbon source in comparison to growth or survival on a C₆ carbon source. Preferably, the chosen candidate inhibitors will significantly inhibit microbial growth or survival on a C₂ carbon source, such as acetate; and will not significantly inhibit microbial growth or survival on a C₆ carbon source, such as glucose.

In certain other preferred embodiments, compounds that inhibit the selected enzyme target(s) are identified by at least a two-part screening technique. In such methods, the candidate compounds are first tested for the fundamental ability to inhibit the activity of the selected enzyme target in a cell-free enzyme activity assay. Positive candidate inhibitors from the enzyme assays are then tested for the ability to inhibit the selected enzyme target in intact cells, preferably when the microbes are grown on a carbon source in vitro that mimics the nutrient environment encountered during the persistent phase of infection in vivo.

A particularly useful practical embodiment of this inventive technique is a screening method that comprises:

(a) testing the ability of a candidate compound to inhibit the activity of a selected enzyme target in a cell-free enzyme activity assay, thereby selecting a positive candidate inhibitor; and

(b) further testing or confirming the ability of the positive candidate inhibitors from the cell-free enzyme activity assay to significantly inhibit the growth of the intracellular microbial pathogen when grown on acetate in vitro, but to not significantly inhibit the growth of the intracellular microbial pathogen when grown under appropriately controlled conditions, but on glucose, thereby favoring the selection of an inhibitor that preferentially inhibits an enzyme of the glyoxylate shunt pathway operative in persistent infection of inflammatory macrophages in vivo.

The invention further provides the motivation to prepare or “grow” crystals of the selected enzyme targets so that their crystal structure can be determined and candidate inhibitors selected or designed from an understanding of such three-dimensional structural information.

Accordingly, the invention provides screening methods wherein a compound that inhibits the selected enzyme target is identified by a method comprising:

(a) preparing a crystal of the selected enzyme target;

(b) obtaining the atomic coordinates of the selected enzyme target by X-ray diffraction studies using the crystal;

(c) using the atomic coordinates to define the catalytic active site of the selected enzyme target; and

(d) identifying or designing a non-native substrate compound that fits the catalytic active site, thereby identifying a candidate compound that inhibits the selected enzyme target.

In exemplary embodiments, the invention provides methods for preparing isocitrate lyase and malate synthase crystals, and provides the resultant crystals and three-dimensional structural information. The crystals and three-dimensional structural information are preferably for microbial enzymes. In certain embodiments, the sources of the enzymes are mycobacterial, such as from M. tuberculosis, and in other embodiments, the sources of the enzymes are fungal, such as from C. albicans.

The crystals and three-dimensional structural information are also preferably for enzymes that include an ordered active site. As such, the invention particularly provides a crystallized mycobacterial, or M. tuberculosis, isocitrate lyase enzyme that includes an ordered active site. In certain embodiments, the invention provides a crystallized microbial isocitrate lyase enzyme other than an isocitrate lyase enzyme from E. coli and other than an isocitrate lyase enzyme from Aspergillus nidulans.

In certain preferred embodiments, the invention provides a crystallized M. tuberculosis isocitrate lyase enzyme without ligand, i.e., apo-ICL, that has the crystallographic data of Table 1. Crystallized apo-ICL that has the crystallographic or atomic coordinates of FIG. 5, as deposited in the Protein Data Bank under accession code 1F61 is another embodiment of the invention.

Further aspects of the invention are the structure of ICL of FIG. 2, and a computer-readable data storage medium comprising a data storage material encoded with computer-readable data, wherein the data comprises the structural information for ICL as represented in FIG. 2.

Additional embodiments of the invention are an ICL tetramer wherein each subunit is composed of 14 α-helices and 14 β-strands; wherein the eight α-helices (α4-α11) and eight β-strands (β2-β5, β8, β12-β14) of the largest domain form an α/β-barrel that has a topology of (βα)₂α(βα)₅β; wherein helix α12, present after the eighth β-strand, projects away from the barrel; wherein helices α12, α13 and α14 form interactions exclusively with the neighboring subunit; wherein residues 184-200 and 235-254 form a small β-domain of a short five-stranded β-sheet (β6, β7, β9, β10, β11) which lies atop the α/β-barrel; and a computer-readable data storage medium comprising a data storage material encoded with computer-readable data, wherein the data comprises the structural information for a so-defined ICL tetramer.

Certain preferred embodiments of the invention are ICL crystals and the three dimensional structure of such ICL crystals in combination with an inhibitor, such as 3-brompyruvate or 3-nitropropionate. In addition to native enzymes, mutant forms of ICL may be used throughout and may prove advantageous in certain embodiments, as shown by the data herein.

Exemplary methods for making crystals of microbial, mycobacterial or M. tuberculosis isocitrate lyase enzymes are provided that comprise:

(a) crystallizing a microbial, mycobacterial or M. tuberculosis isocitrate lyase enzyme by vapor diffusion using a buffer comprising about 100 mM HEPES and about 1.4 M sodium citrate at about pH 7.5; or

(b) incubating a microbial, mycobacterial or M. tuberculosis isocitrate lyase enzyme with glyoxylate, modifying the incubated enzyme with 3-bromopyruvate and crystallizing the modified enzyme in the presence of a buffer comprising about 0.1 M Tris-HCl, about 0.2 M sodium acetate, about 20-30% PEG4000 at about pH 8.0.

In terms of inhibitor complexes, the invention particularly provides crystallized ICL in complex with the inhibitor 3-brompyruvate that has the crystallographic or atomic coordinates of FIG. 6, as deposited in the Protein Data Bank under accession code 1F8M; and crystallized ICL in complex with the inhibitor 3-nitropropionate that has the crystallographic or atomic coordinates of FIG. 7, as deposited in the Protein Data Bank under accession code 1F8I.

The invention also provides the structures of the bound complexes of FIG. 3, and a computer-readable data storage medium comprising a data storage material encoded with computer-readable data, wherein the data comprises the structural information for the bound complexes of FIG. 3.

Further embodiments of the invention are thus structural determinants of a mutant 1CI in which Cys 191 is changed to Ser and in which a crystal is formed with glyoxylate or 3-nitropropionate, such that the glyoxylate binds by coordination to the active site Mg²⁺ ion and forms hydrogen bonds with residues Ser 91 OG, Gly 92 N, Trp 93 N and Arg 228 NH2; structural determinants in which a succinate molecule is fit to the density such that one carboxylate makes specific hydrogen bonds with the side chains of residues Asn 313 ND1, Glu 295 OE2, Arg 228 NH1 and Gly 192 N, while the second carboxylate forms hydrogen bonds with Thr 347 OG, Asn 313 ND2, Ser 315 OG, Ser 317 OG and His 193 ND1, wherein the protein surface that packs against the C2 and C3 methylene carbons of succinte is provided by residues Trp 93, Thr 347 and Leu 348, and the C2 carbon of succinate is located 3.9 Å from the aldehyde carbon of glyoxylate and 3.2 Å from the hydroxyl of Ser 191 of the C191S mutant; and a computer-readable data storage medium comprising a data storage material encoded with computer-readable data, wherein the data comprises the so-defined structural determinants.

The invention yet further provides the structures of the active site of ICL of FIG. 4, and a computer-readable data storage medium comprising a data storage material encoded with computer-readable data, wherein the data comprises the structural information for the active site of ICL of FIG. 4.

Still further embodiments of the invention are thus structural determinants of ICL under inhibition by 3-bromopyruvate, wherein inhibition is accomplished via dehalogenation of the inhibitor to form a covalent adduct with active site nucleophile, Cys 191; wherein the pyruvyl moiety occupies the site where the second carboxylate of succinate was located and forms hydrogen bonds with the side chains of His 193 ND1, Asn 313 ND2, Ser 315 OG, Ser 317 OG, Thr 347 OG1 and a solvent molecule; and a computer-readable data storage medium comprising a data storage material encoded with computer-readable data, wherein the data comprises the so-defined structural determinants of ICL under inhibition by 3 -bromopyruvate.

Additional aspects of the present invention include structural information on conformational changes of ICL, particularly in two regions that control access to the active site; including information on the ‘open’ conformation of the apoenzyme and the ‘closed’ conformation adopted upon binding of inhibitor, and on interactions connected with obtaining the closed conformation, such as interactions connected with loop closure in the pocket formed by residues His 193, Asn 313, Ser 315, Ser 317, and Thr 347, wherein residues His 193, Asn 313, Ser 315 and Ser 317 undergo significant movements upon binding; and a computer-readable data storage medium comprising a data storage material encoded with computer-readable data, wherein the data comprises the so-defined structural information on conformational changes, active site and interactions connected with obtaining the closed conformation.

Particular embodiments of the invention are computer-readable data storage media that comprise data storage materials encoded with computer-readable data, wherein the data comprises the ICL crystal structure as defined in Table 1; the crystal structure coordinates for apo-ICL, as shown in FIG. 5 and deposited in the Protein Data Bank under accession code 1F61; the crystal structure coordinates for the 3-brompyruvate-ICL complex, as shown in FIG. 6 and deposited in the Protein Data Bank under accession code 1F8M; the crystal structure coordinates for the 3-nitropropionate-ICL complex, as shown in FIG. 7 and deposited in the Protein Data Bank under accession code 1F8I; and/or the crystal structure coordinates of ICL amino acids His 193, Asn 313, Ser 315 and Ser 317 according to FIG. 5, FIG. 6 or FIG. 7.

Certain preferred computer-readable data storage media are those comprising a data storage material encoded with computer-readable data, wherein the data comprises the structure coordinates of ICL amino acids His 193, Asn 313, Ser 315 and Ser 317 according to FIG. 5, FIG. 6 or FIG. 7; wherein amino acids His 193, Asn 313, Ser 315 and Ser 317 mediate closure of the active site loop upon binding of an inhibitor to ICL.

Further aspects of the invention are computerized formats of relevant crystal structure data and computers for producing three-dimensional representations of enzymes important in the development of inhibitors for use in treating persistent microbial infections. Computerized formats of the preferred crystal structure data and computers for producing three-dimensional representations of microbial glyoxylate shunt enzymes, preferably ICL and malate synthase are particularly provided.

In light of the technical skill in this particular art, those of ordinary skill will be able to make and use suitable computer-readable data storage media and computers relevant to the presently claimed invention without undue experimentation. Nonetheless, U.S. Pat. No. 6,183,121 is specifically incorporated herein by reference for purposes including further describing and enabling those aspects of the claimed invention concerning computer-readable data storage media and computers.

One example of these embodiments is a computer for producing a three-dimensional representation of:

(a) a molecule or molecular complex comprising the structure coordinates of FIG. 5 (Protein Data Bank accession code 1F61), FIG. 6 (Protein Data Bank accession code 1F8M) or FIG. 7 (Protein Data Bank accession code 1F8I);or

(b) a homologue of such a molecule or molecular complex, wherein the homologue comprises structure coordinates that have a root mean square deviation from the backbone atoms of the amino acids of the structure coordinates of FIG. 5, FIG. 6 or FIG. 7 of not more than 1.5 angstroms; wherein the computer comprises:

(i) a computer-readable data storage medium comprising a data storage material encoded with computer-readable data, wherein the data comprises the structure coordinates of FIG. 5, FIG. 6 or FIG. 7;

(ii) a working memory for storing instructions for processing the computer-readable data;

(iii) a central-processing unit coupled to the working memory and to the computer-readable data storage medium for processing the computer-machine readable data into the three-dimensional representation; and

(iv) a display coupled to the central-processing unit for displaying the three-dimensional representation.

A further example is a computer for producing a three-dimensional representation of:

(a) a molecule or molecular complex that comprises an active site loop defined by the structure coordinates of ICL amino acids His 193, Asn 313, Ser 315 and Ser 317 according to FIG. 5, FIG. 6 or FIG. 7; wherein amino acids His 193, Asn 313, Ser 315 and Ser 317 mediate closure of the active site loop upon binding of an inhibitor to ICL; or

(b) a homologue of such a molecule or molecular complex, wherein the homologue comprises an active site loop that has a root mean square deviation from the backbone atoms of the foregoing amino acids of not more than 1.5 angstroms, wherein the computer comprises:

(i) a computer-readable data storage medium comprising a data storage material encoded with computer-readable data, wherein the data comprises the structure coordinates of ICL amino acids His 193, Asn 313, Ser 315 and Ser 317 according to FIG. 5, FIG. 6 or FIG. 7;

(ii) a working memory for storing instructions for processing the computer-readable data;

(iii) a central-processing unit coupled to the working memory and to the computer-readable data storage medium for processing the computer-machine readable data into the three-dimensional representation; and

(iv) a display coupled to the central-processing unit for displaying the three-dimensional representation.

Yet another example is a computer for determining at least a portion of the structure coordinates corresponding to X-ray diffraction data obtained from a molecule or molecular complex, wherein the computer comprises:

(a) a computer-readable data storage medium comprising a data storage material encoded with machine-readable data, wherein the data comprises at least a portion of the structural coordinates of apo-ICL according to FIG. 5 (Protein Data Bank accession code 1F61), ICL-3-brompyruvate complex according to FIG. 6 (Protein Data Bank accession code 1F8M) or ICL-3-nitropropionate complex according to FIG. 7 (Protein Data Bank accession code 1F8I);

(b) a computer-readable data storage medium comprising a data storage material encoded with computer-readable data, wherein the data comprises X-ray diffraction data obtained from the molecule or molecular complex;

(c) a working memory for storing instructions for processing the computer-readable data of (a) and (b);

(d) a central-processing unit coupled to the working memory and to the computer-readable data storage medium of (a) and (b) for performing a Fourier transform of the machine readable data of (a) and for processing the computer-readable data of (b) into structure coordinates; and

(e) a display coupled to the central-processing unit for displaying the structure coordinates of the molecule or molecular complex.

In certain tspects, the computer and methods of operation therewith, the computer-readable data storage medium will comprise a data storage material encoded with machine-readable data that comprises the structural coordinates of apo-ICL according to FIG. 5 (1F61 ) ICI-3-brompyruvate complex according to FIG. 6 (1F8M) and/or ICL-3-nitropropionate complex according to FIG. 7 (1F8I). In other aspects, the molecule or molecular complex will comprise or represent a polypeptide having isocitrate lyase enzyme activity.

Returning to the methodological aspects of the present invention, there are also provided various methods for identifying inhibitors of selected enzyme targets from microbial, mycobacterial, M. tuberculosis or fungal glyoxylate shunt pathways, preferably isocitrate lyase or malate synthase enzymes. Such methods comprise delineating the crystal structure of microbial, mycobacterial or M. tuberculosis or fungal isocitrate lyase or malate synthase to provide crystal structure information for the active site, and identifying a non-native substrate compound that fits the active site, wherein a non-native substrate compound that fits the active site is indicative of an inhibitor of a microbial, mycobacterial or M. tuberculosis or fungal isocitrate lyase or malate synthase enzyme.

In the methods of the invention, the entire range of crystals, crystal structure information, data carriers and computers may be employed, as described hereinabove and throughout the present application. Although the following methods are succinctly described in terms of certain preferred and exemplary embodiments, it will be understood that the methods apply to the entire breadth of crystals and atomic coordinate information and related compositions and apparatus. As such, enzymes may be native or mutant, and the primary information may have been gathered from crystals of the apo-forms of the enzymes or the enzymes in complexes with inhibitors.

The invention provides, as an example, methods of using the structural information in one or more of FIG. 1, FIG. 2, FIG. 3, FIG. 4, FIG. 5 (1F61), FIG. 6 (1F8M) and FIG. 7 (1F8I), and/or computer-readable data storage media comprising a data storage material encoded with computer-readable data, wherein the data comprises the structural information in one or more of FIG. 1, FIG. 2, FIG. 3, FIG. 4, FIG. 5 (1F61), FIG. 6 (1F8M) and FIG. 7 (1F8I), to identify, design or develop new compounds, and/or to adapt, modify or refine existing compounds, which compounds inhibit the biochemical activity of ICL. These methods are particularly suitable to identify, design, develop, adapt and/or refine compounds that inhibit the biochemical activity of ICL during the persistent phase of microbial infections, such as mycobacterial infection, and particularly, persistent infection by M. tuberculosis.

These methods of identifying or designing inhibitors comprise using the structural information in one or more of FIG. 1, FIG. 2, FIG. 3, FIG. 4, FIG. 5 (1F61), FIG. 6 (1F8M) and FIG. 7 (1F8I), or computer-readable data comprising such structural information, to develop one or more compounds that bind to ICL.

Such methods generally comprise the steps of:

(a) obtaining the structural information for the ICL enzyme in one or more of FIG. 1, FIG. 2, FIG. 3, FIG. 4, FIG. 5 (1F61), FIG. 6 (1IF8M) and FIG. 7 (1F8I), or computer-readable data comprising such structural information; and

(b) using the structural information to identify and/or design a compound that will bind to and inhibit the activity of the ICL enzyme, e.g., by using the structural information to identify and/or design a compound that fits the active or catalytic site of the ICL enzyme, such that the compound will bind to the active or catalytic site and thereby inhibit the enzyme.

Additional methods of the invention are methods for evaluating the potential of a candidate compound to associate with:

(a) a molecule or molecular complex that comprises an active site loop defined by the structure coordinates of ICL amino acids His 193, Asn 313, Ser 315 and Ser 317 according to FIG. 5, FIG. 6 or FIG. 7; or

(b) a homologue of the molecule or molecular complex, wherein the homologue comprises an active site loop that has a root mean square deviation from the backbone atoms of the amino acids of not more than 1.5 angstroms; wherein the method comprises the steps of:

(i) employing computational means to perform a fitting operation between the candidate compound and an active site loop defined by the structure coordinates of ICL amino acids His 193, Asn 313, Ser 315 and Ser 317 according to FIG. 5, FIG. 6 or FIG. 7 within a root mean square deviation from the backbone atoms of such amino acids of not more than 1.5 angstroms; and

(ii) analyzing the results of the fitting operation to quantify the association between the candidate compound and the active site loop.

Such methods may be used to evaluate the potential of the candidate compound to associate with:

(a) a molecule or molecular complex defined by structure coordinates of all amino acids of ICL, wherein the structure coordinates are selected from the group consisting of apo-ICL according to FIG. 5 (1F61), ICL-3-brompyruvate complex according to FIG. 6 (1F8M) and ICL-3-nitropropionate complex according to FIG. 7 (1F8I); or

(b) a homologue of the molecule or molecular complex having a root mean square deviation from the backbone atoms of the amino acids of not more than 1.5 angstroms.

Further methods are those for identifying an inhibitor of a microbial isocitrate lyase enzyme (ICL), comprising:

(a) defining the catalytic active site of ICL from the atomic coordinates selected from the group consisting of the atomic coordinates for the apo-ICL of FIG. 5 (Protein Data Bank accession code 1F61), the atomic coordinates for the ICL-3-brompyruvate complex of FIG. 6 (Protein Data Bank accession code 1F8M) and the atomic coordinates for the ICL-3-nitropropionate complex of FIG. 7 (Protein Data Bank accession code 1F8I); and

(b) identifying a non-native substrate compound that fits the active site and thereby inhibits the microbial isocitrate lyase enzyme.

Additional methods for identifying an inhibitor of a microbial ICL, comprise:

(a) obtaining atomic coordinates of ICL, wherein the atomic coordinates are selected from the group consisting of the apo-ICL atomic coordinates of FIG. 5 (Protein Data Bank accession code 1F61), the ICL-3-brompyruvate complex atomic coordinates of FIG. 6 (Protein Data Bank accession code 1F8M) and the ICL-3-nitropropionate complex atomic coordinates of FIG. 7 (Protein Data Bank accession code 1F8I);

(b) defining the catalytic active site of ICL from the atomic coordinates; and

(c) identifying a non-native substrate compound that fits the catalytic active site, wherein a non-native substrate compound that fits the active site is indicative of an inhibitor of a microbial ICL.

In these methods, the non-native substrate compound that fits the catalytic active site should preferably be identified or confirmed, e.g., by selecting a candidate compound and confirming that the candidate compound inhibits the microbial ICL.

Further embodiments that exemplify the generic aspects of the claimed invention with respect to methods to identify inhibitors of microbial ICL, comprise:

(a) obtaining atomic coordinates of ICL, wherein the atomic coordinates are selected from the group consisting of the apo-ICL atomic coordinates of FIG. 5 (Protein Data Bank accession code 1F61), the ICL-3-brompyruvate complex atomic coordinates of FIG. 6 (Protein Data Bank accession code 1F8M) and the ICL-3-nitropropionate complex atomic coordinates of FIG. 7 (Protein Data Bank accession code 1F8I);

(b) defining the catalytic active site of ICL from the atomic coordinates;

(c) selecting a candidate compound by identifying a non-native substrate compound that fits the catalytic active site; and

(d) contacting the microbial ICL with the candidate compound under conditions effective for ICL activity, wherein a candidate compound that inhibits the activity of the microbial ICL is confirmed as an inhibitor of the microbial ICL.

In reference to ICL, simply by means of an example, the invention provides methods for identifying a potential agonist or antagonist of a molecule comprising a microbial ICL-like active site loop, wherein such methods comprise:

(a) using the atomic coordinates of ICL amino acids His 193, Asn 313, Ser 315 and Ser 317 according to FIG. 5, FIG. 6 or FIG. 7 within a root mean square deviation from the backbone atoms of the amino acids of not more than 1.5 angstroms, to generate a three-dimensional structure of molecule comprising a microbial ICL-like active site loop;

(b) employing the three-dimensional structure to design or select the potential agonist or antagonist;

(c) purifying or synthesizing the potential agonist or antagonist; and

(d) contacting the potential agonist or antagonist with the molecule to determine the ability of the potential agonist or antagonist to interact with the molecule.

Use of the atomic coordinates of all the amino acids of ICL according to FIG. 5, FIG. 6 or FIG. 7 within a root mean square deviation from the backbone atoms of the amino acids of not more than 1.5 angstroms is a further embodiment.

As tuberculosis is a widespread and fatal disease, certain methods of the invention are particularly adapted for use in identifying agents for use in the treatment of chronic tuberculosis, and as such comprise:

(a) obtaining atomic coordinates of M. tuberculosis ICL, wherein the atomic coordinates are selected from the group consisting of the apo-ICL atomic coordinates of FIG. 5 (Protein Data Bank accession code 1F61), the ICL-3-brompyruvate complex atomic coordinates of FIG. 6 (Protein Data Bank accession code 1F8M) and the ICL-3-nitropropionate complex atomic coordinates of FIG. 7 (Protein Data Bank accession code 1F8I);

(b) defining the catalytic active site of ICL from the atomic coordinates;

(c) selecting a candidate compound by identifying a non-native substrate compound that fits the catalytic active site; and

(d) contacting M. tuberculosis ICL with the candidate compound under conditions effective for ICL activity, wherein a candidate compound that inhibits the activity of the M. tuberculosis ICL is indicative of an agent for use in the treatment of chronic tuberculosis.

Candidate or potential inhibitors may prove to be actual inhibitors by binding to the isocitrate lyase or malate synthase enzymes at various points. However, it is particularly contemplated that many useful inhibitors will be non-native substrate compounds that fit the active site and that, upon binding of the inhibitor to the enzyme, change the structure of the enzyme from the open conformation to the closed conformation. As such, one may wish to pre-select or design candidate inhibitors so that they are intended to interact with at least one amino acid residue involved in the open to closed structural transition.

In terms of ICL, one may wish to pre-select or design candidate inhibitors so that they are intended to interact with at least one of amino acid residues His 193, Asn 313, Ser 315 or Ser 317 in the structure of isocitrate lyase from M. tuberculosis, or the equivalent amino acids in the structures of isocitrate lyase from other organisms. The candidate compound may also be selected by identifying a compound intended to interact with at least one of ICL amino acids His 193, Asn 313, Ser 315 or Ser 317 according to FIG. 5, FIG. 6 or FIG. 7. Such selection steps employ computational means to perform a fitting operation between the candidate compound and a binding pocket defined by the foregoing amino acids within a root mean square deviation from the backbone atoms of the amino acids of not more than 1.5 angstroms.

Once crystal structure information is available, whether for ICL, malate synthase or other selected enzyme targets from a microbial glyoxylate shunt pathway, there is a high level of technical skill in the art concerning the identification and/or design of inhibitors. One of ordinary skill in the art will thus be able to identify and/or design appropriate inhibitors in light of the present disclosure without undue experimentation Nonetheless, U.S. Pat. No. 6,057,119 is specifically incorporated herein by reference for purposes including further describing and enabling those aspects of the claimed invention concerning the origin of candidate inhibitors and the identification and/or design of actual inhibitors.

The candidate compounds may thus be selected from consideration of a database of compounds, selected by de novo design and/or selected by design starting from a known inhibitor, as described in U.S. Pat. No. 6,057,119. Candidate inhibitors designed starting from complete or partial structural information for a known inhibitor are particularly advantageous in the presently claimed invention, given the detailed structural information for ICL-inhibitor complexes provided herein.

Again, whether conducted in reference to ICL, malate synthase or other selected enzyme targets from a microbial glyoxylate shunt pathway, once a candidate inhibitor has been identified, the invention provides various methods of perfecting such a candidate inhibitor for development into a therapeutic. Naturally, if the inhibitor has been selected from known compounds or databases or derived from related molecules, the inhibitor should be purified. Where the inhibitor has been designed from first principles, e.g., using computer modeling, the inhibitor should be synthesized (and then purified if necessary).

In the execution of the methods, the candidate or potential inhibitor compounds identified from crystal structure considerations should preferably be confirmed as actual inhibitors by biochemical studies. Generally, the candidate compound should be purified or synthesized and the ability of the candidate compound to inhibit the selected enzyme target should be confirmed in a cell-free, in vitro or in vivo assay or combination thereof.

Suitable confirmatory methods would generally comprise contacting the enzyme with the candidate inhibitor compound under conditions appropriate for enzyme activity, i.e., at least in the presence of substrate and under suitable temperature, pH and the like, and determining the inhibition of the enzymatic activity of the enzyme by the candidate compound. Either by design in advance, and/or by testing after identification, the inhibitors identified by the present invention may be competitive, non-competitive or uncompetitive inhibitors.

A “competitive” inhibitor is one that inhibits enzyme activity by binding the same kinetic form of the enzyme as its substrate binds, thus directly competing with the substrate for the active site of the enzyme. Competitive inhibition can be reversed completely by increasing the substrate concentration. An “uncompetitive” inhibitor is one that inhibits an enzyme by binding to a different kinetic form of the enzyme than does the substrate. Such inhibitors bind to enzymes already bound with the substrate and not to the free enzyme. Uncompetitive inhibition cannot be reversed completely by increasing the substrate concentration. A “non-competitive” inhibitor is one that can bind to either the free or substrate bound form of an enzyme. One of ordinary skill in the art may identify inhibitors as competitive, uncompetitive or non-competitive, by computer fitting enzyme kinetic data, e.g., using standard equations according to Segel (1975), specifically incorporated herein by reference.

Following appropriate confirmatory studies, to prepare agents for use in treating or preventing persistent microbial infections, the compounds so identified should be purified or synthesized, as appropriate, and then formulated in a pharmaceutically acceptable formulation. Optionally, such pharmaceutically acceptable formulations may further comprise at least a second antimicrobial agent.

In yet further embodiments, the invention provides the inhibitors identified and prepared by any one or more of the foregoing methods.

In still other embodiments, the invention provides methods of inhibiting selected enzyme targets of the glyoxylate shunt pathway of a pathogenic microbe, such as ICL or malate synthase. These methods first comprise identifying, obtaining and/or preparing one or more compounds capable of inhibiting selected enzyme targets of the glyoxylate shunt pathway of a pathogenic microbe, e.g., by any one or more of the foregoing methods. The methods then comprise contacting the selected enzyme target, or a composition comprising at least one of the selected enzyme targets, with the inhibitory compound so identified in an amount and for a time effective to inhibit the activity of the selected enzyme target.

The “contacting” is generally performed so that the inhibitory compound contacts the selected enzyme target under conditions in which the enzyme would otherwise be active, but for the presence of the inhibitory compound. For example, the inhibitory compound is provided to the selected enzyme target in the presence of appropriate substrates, and any cofactors, etc., and at appropriate temperatures, pH, and other conditions suitable for enzyme activity. “Contacting”, in this context, means providing the compound in an effective amount and for an effective period of time, i.e., in amounts and for times effective to result in enzyme inhibition.

Where the selected enzyme target to be so inhibited is within a microorganism, the invention further provides methods of inhibiting the growth of, or killing, such a microorganism. Such methods comprise identifying, obtaining and/or preparing one or more compounds capable of inhibiting selected enzyme targets of the glyoxylate shunt pathway of a pathogenic microbe, e.g., by any one or more of the foregoing methods, and contacting a microorganism that comprises the selected enzyme target with an amount of the inhibitory compounds effective to inhibit the selected enzyme target, thereby inhibiting the growth of, or killing, the microorganism.

Where the microorganism that comprises the selected enzyme target is located within an animal or patient, the invention further provides methods for treating a microbial infection, particularly a chronic or persistent microbial infection. Such methods comprise identifying, obtaining and/or preparing one or more compounds capable of inhibiting a selected enzyme target of the glyoxylate shunt pathway of a pathogenic microbe, e.g., by any one or more of the foregoing methods, and providing the inhibitory compound to an animal or patient having, or suspected of having, a microbial infection in an amount effective to inhibit the selected enzyme target within the microorganism, thereby treating the microbial infection, and particularly treating a chronic or persistent microbial infection.

The present invention further provides preventative and prophylactic methods, comprising identifying, obtaining and/or preparing one or more compounds capable of inhibiting a selected enzyme target of the glyoxylate shunt pathway of a pathogenic microbe, e.g., by any one or more of the foregoing methods; and providing the inhibitory compound to an animal or patient at risk of developing a microbial infection in an amount effective to inhibit the selected enzyme target within a microorganism, thereby preventing or reducing the severity and/or duration of microbial infection in the at risk animal or patient.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A, FIG. 1B, FIG. 1C, FIG. ID, FIG. 1E, FIG. 1F and FIG. 1G. Inhibition of ICL activity. FIG. 1A, the reaction catalyzed by ICL can be inhibited by 3-bromopyruvate (FIG. 1B) and 3-nitropropionate (FIG. 1C). FIG. 1D, FIG. 1E, FIG. 1F and FIG. 1G, the inhibitory effects of 3-nitropropionate on both wild type and a pICL1 complemented mutant strain of M. smegmatis (ICL from M. smegmatis replaced with M. tuberculosis) are restricted to growth on acetate and are not observed on glucose. The drug discs shown in each of FIG. 1D, FIG. 1E, FIG. 1F and FIG. 1G are saturated with 30 mM and 60 mM nitropropionate. The M. smegmatis wild type was grown on glucose (FIG. 1D) or acetate (FIG. 1F) and the M. smegmatis Δicl mutant was defective for growth on fatty acids was rescued by complementation with pICL1 (Example 2; McKinney et al., 2000) and grown on glucose (FIG. 1E) or acetate (FIG. 1G).

FIG. 2A, FIG. 2B and FIG. 2C. The structure of ICL. FIG. 2A, ribbon representation of the ICL homotetramer, with each subunit is colored differently. The four subunits of the tetramer are related by 222 symmetry. The cyan and blue and the yellow and green subunits show extensive interactions, primarily via helix-swapping. FIG. 2B and FIG. 2C, stereo views of a subunit. The α-helices are shown in yellow and the β-strands are shown in purple.

FIG. 3A, FIG. 3B, FIG. 3C, FIG. 3D and FIG. 3E. Binding of 3-nitropropionate and glyoxylate. FIG. 3A and FIG. 3B, stereo view of the active site of the ternary complex of the ICL C191S mutant with glyoxylate (GA) and 3-nitropropionate (shown as succinate, SA). Since the ambiguity of the nitro group is unresolved, succinate is used to depict the 3-nitropropionate in figures and the text. The carbon atoms are shown in yellow (protein), green (GA) or cyan (SA). FIG. 3C and FIG. 3D, stereo view of the NCS averaged difference Fourier maps, (|Fₒ|-|F_{c}|φ_{c}) contoured at the 2σ level with glyoxylate (green), succinate (cyan), Mg²⁺ ion (yellow) and three waters (red) in the active site of ICL. FIG. 3E, schematic diagram of ICL interactions with glyoxylate and succinate.

FIG. 4A, FIG. 4B, FIG. 4C and FIG. 4. Active site of ICL. FIG. 4A and FIG. 4B, stereo view of the 2|Fₒ|-|F_{c}|φ_{c} electron density map contoured at 1σ for bound 3-bromopyruvate modified ICL. Shown is the active site with the pyruvyl moiety (purple) attached to the thiolate of Cys 191. FIG. 4C and FIG. 4, stereo view of active site of ICL shown as a molecular surface colored according to the electrostatic surface potential. The surface was generated based on the protein coordinates of the ternary complex of ICL with glyoxylate (green) and succinate (cyan). The atoms of the active site loop (residues 183-197) and the C-terminal segment (residues 410-427) of the adjacent subunit were excluded during surface calculations. The loop segments are shown as yellow ribbon for the inhibitor complex and white ribbon for the apo enzyme. Side-chains of some of the residues have been omitted for clarity, as is standard in this art.

FIG. 5-1 though FIG. 5-97. Coordinates for apo-ICL. The coordinates for apo-ICL have been deposited in the Protein Data Bank (accession code 1F61 for apo-ICL).

FIG. 6-1 through FIG. 6-187. Coordinates for the 3-brompyruvate-ICL complex. The coordinates for the 3-brompyruvate-ICL complex have been deposited in the Protein Data Bank (accession code 1F8M for the 3-brompyruvate complex).

FIG. 7-1 through FIG. 7-189. Coordinates for the 3-nitropropionate-ICL complex. The coordinates for the 3-nitropropionate-ICL complex have been deposited in the Protein Data Bank (accession code 1F8I for the 3-nitropropionate complex).

### DESCRIPTION OF ILLUSTRATIVE EMBODIMENTS

### I. Inhibitors of the Glyoxylate Shunt

Many pathogenic microbes, including Mycobacteria such as M. tuberculosis, cause persistent infections that are difficult to treat effectively. In TB infections, mycobacteria are able to colonize macrophages throughout the course of host's immune response This progression is delineated by rapid replication of bacteria in the naive host, to the maintenance of chronic infection while confronting an effective immune response. Studies on macrophages in culture indicate that this transition is regulated by activation of host macrophages, which leads to exposure of M. tuberculosis to less hospitable environments (Schaible et al., 1998).

Despite some understanding of the effects of environmental changes on bacterial metabolism in culture, prior to the present invention, there was minimal appreciation of the impact of these enviromnental shifts on intracellular pathogens. In order to develop more effective drugs for use in treating persistent infection, the inventors realized that it was imperative to expand the knowledge of the plasticity of bacterial metabolism during the course of infection. Through an enhanced and rigorously tested understanding of the mechanisms and pathways involved in persistence, the inventors are now able to provide important targets and methodology for developing and/or refining anti-mycobacterial agents.

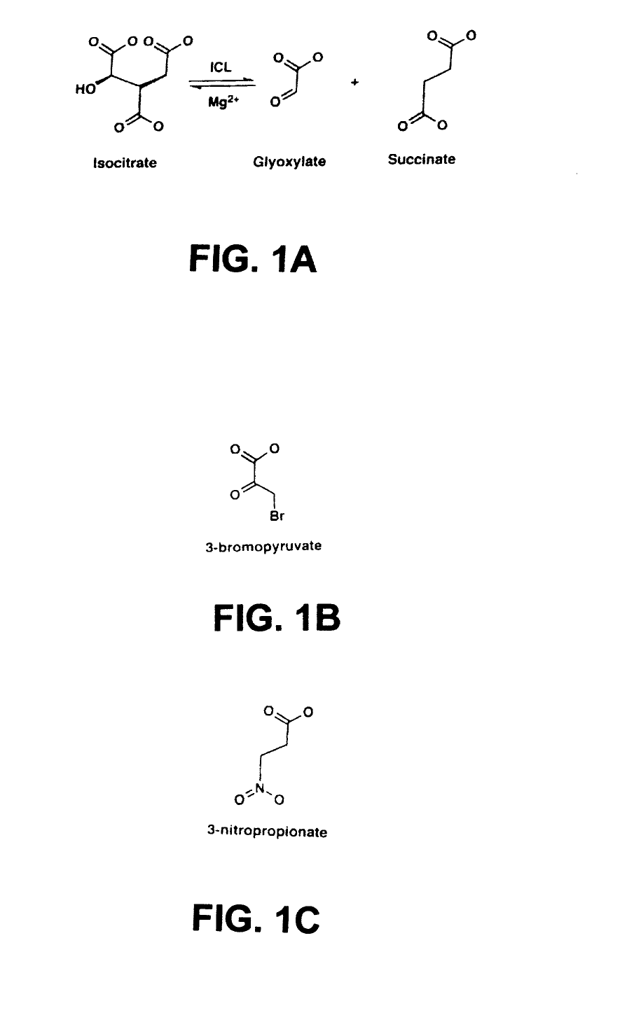

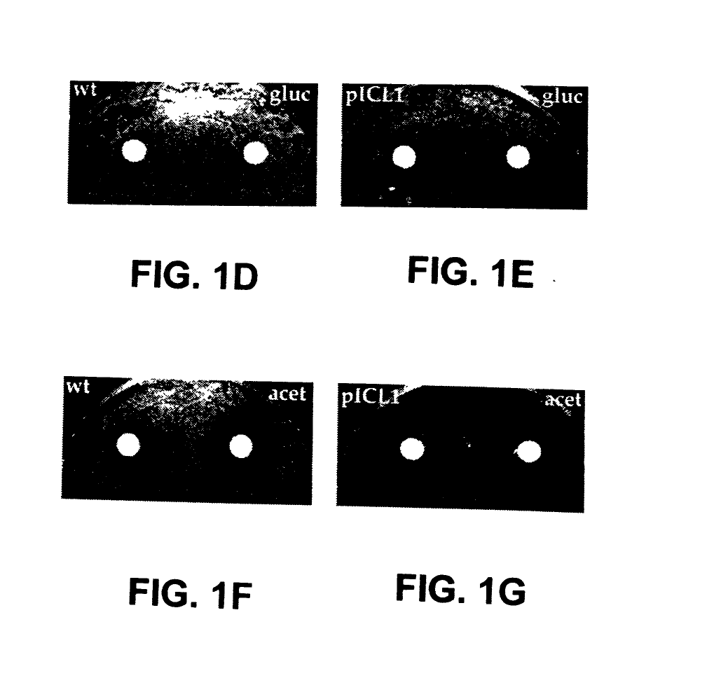

Persistent mycobacteria not only adapt to the adverse environment within the macrophage, but also survive killing by potent anti-mycobacterial or anti-TB drugs. The strategy for survival during chronic stages of infection entails a metabolic shift in the bacteria's carbon source to C₂ substrates generated by β-oxidation of fatty acids (Segal, 1984). Under these conditions, glycolysis is decreased and the glyoxylate shunt is significantly upregulated to allow anaplerotic maintenance of the TCA cycle and assimilation of carbon via gluconeogenesis (Wheeler and Ratledge, 1988). The glyoxylate shunt accomplishes this by converting isocitrate to succinate and glyoxylate (FIG. 1A) by isocitrate lyase (ICL: threo-D_{S}-isocitrate-glyoxylate-lyase; EC 4.1.3.1), followed by addition of acetyl-CoA to glyoxylate to form malate by malate synthase.

The carbon conserving glyoxylate pathway is present in most prokaryotes, lower eukaryotes and plants, but has not been observed in vertebrates (Vanni et al., 1990). In plants, it serves to utilize seed lipids for growth, and in microorganisms it provides a means to survive on fatty acids as the sole carbon source. Highly elevated levels of ICL are observed in Mycobacterium spp. grown on C₂ sources (Hoener zu Bentrup et al., 1999), and shortly after uptake into human macrophages (Graham and Clark-Curtiss, 1999).

In order to identify better targets for drug development, particularly for the development of agents to combat persistent infection, the inventors decided to focus on the glyoxylate shunt pathway. The present invention is therefore founded, at least in part, upon the realization that enzymes of the glyoxylate shunt pathway in pathogenic organisms are ideal targets for use in developing therapeutics to combat persistent infections, and particularly on the rigorous validation of these concepts. The inventors realized that enzymes of the glyoxylate shunt pathway, such as isocitrate lyase (ICL) and malate synthase, play a pivotal role in persistent infection by a number of pathogens, including Mycobacterium tuberculosis, by sustaining intracellular infection, e.g., in inflammatory macrophages. In light of the present invention, these enzymes can now be used in both screening and rational drug design to identify, design and/or refine inhibitors for therapeutic use.

Initial data towards the invention concerned the characterization of activity and expression of isocitrate lyase in M. avium and M. tuberculosis (Example 1; Honer Zu Bentrup et al., 1999; specifically incorporated herein by reference). Analysis by two-dimensional gel electrophoresis revealed that M. avium expressed several proteins unique to an intracellular infection. One abundant protein with an apparent molecular mass of 50 kDa was isolated, and the N-terminal sequence was determined. This sequence matched a sequence in the M. tuberculosis database with similarity to isocitrate lyase of both Corynebacterium glutamicum and Rhodococcus fascians.

Only marginal similarity was observed between this open reading frame (ORF) (termed icl) and a second, distinct ORF (named aceA), which exhibits a low similarity to other isocitrate lyases. Both ORFs can be found as distinct genes in the various mycobacterial databases. By expression and purification of ICL and AceA proteins, both were shown to exhibit isocitrate lyase activity and to be effectively inhibited by various known isocitrate lyase inhibitors. Initial evidence was generated to suggest that in both M. avium and M. tuberculosis the production and activity of isocitrate lyase is enhanced under minimal growth conditions when supplemented with acetate or palmitate (Example 1; Honer Zu Bentrup et al., 1999; specifically incorporated herein by reference).

Important data to validate the role of the glyoxylate shunt in persistent infection involved the demonstration that disruption of the gene for ICL (icl) attenuates persistence of M. tuberculosis in mice and inflammatory macrophages (Example 2; McKinney et al., 2000; specifically incorporated herein by reference). While the deletion of ICL did not affect bacterial growth in immunocompromised mice during the acute phase of infection (0-2 wk), significant reduction in the bacterial loads and macroscopic lesions were observed during the chronic phase (2-16 wk). A connection between the expression of ICL in mycobacterium and the immune status of the host was established by the restored virulence of Aicl bacteria in interferon-γ knockout mice. This link was apparent at the level of the infected macrophage as activation of infected macrophages increased expression of ICL, and the Δicl mutant was markedly attenuated for survival in activated, but not resting macrophages. These data indicate that the metabolism of M. tuberculosis in vivo is profoundly influenced by the host response to infection, an observation with important implications for the treatment of chronic tuberculosis.

Thus, according to the present invention, the enzymes of the glyoxylate shunt can be used as targets to develop or refine inhibitors for use in effective antimicrobial strategies to combat persistent infection. Particularly suitable enzyme targets are isocitrate lyase and malate synthase. These are most preferred as they are their activities are specific and limited to the glyoxylate shunt.

However, the invention is not limited to isocitrate lyase and malate synthase, and the malate dehydrogenase, citrate synthase and aconitase enzymes may also be used as targets for drug screening and/or design and ultimate inhibition as part of this invention. Naturally, those of ordinary skill in the art will understand that where isoenzymes are present, the isoenzyme of the glyoxylate shunt should be employed. This is exemplified by malate dehydrogenase, where the cytosolic malate dehydrogenase of the glyoxylate shunt is particularly relevant, in contrast to the mitochondrial or peroxisomal forms.

Although the isocitrate lyase, malate synthase, malate dehydrogenase, citrate synthase and aconitase enzymes of the glyoxylate shunt itself are the primary focus of the invention, and most preferably isocitrate lyase and malate synthase, other enzymes functionally related to this metabolic shunt are by no means excluded from use. For example, acetyl CoA synthase, fructose-1,6-bisphosphatase and other acetyltransferases and transporters associated with the integration of the glyoxylate shunt with other metabolic pathways may be used.

The ICL enzyme allows net carbon gain by diverting acetyl-CoA from β-oxidation of fatty acids into the glyoxylate shunt pathway. Given its role as a drug target against persistent infections, newly validated by the gene disruption data of this invention, the inventors also solved the structure of ICL without ligand (in the apo-form) and in complex with two inhibitors. Covalent modification of an active site residue, Cys 191, by the inhibitor 3-bromopyruvate traps the enzyme in a catalytic conformation with the active site completely inaccessible to solvent. The structure of a C191S mutant of the enzyme with the inhibitor 3-nitropropionate, in particular, provides further insight into the chemical mechanism.

The crystal structure data encompassed by this invention may be advantageously used in the development of antimicrobial agents. However, the development of ICL inhibitors as novel drug candidates with preferential activity against persistent bacteria and fungi does not rest solely on such three-dimensional structures, as such inhibitors can now be identified through standard screening and other biochemical techniques, given the motivation provided by the present data.

Nonetheless, the structures of the inhibitor complexes of the invention provide especially meaningful guidance for the development of drugs to target and inhibit ICL, which drugs would combat the chronic stages of various infections, including tuberculosis. These three dimensional structures allow an understanding of the interactions between the enzyme and inhibitors, which enables those of ordinary skill in the art to utilize rational mechanism-based and structure-based drug design technology to develop specific inhibitors for use as novel drugs. In addition to the design of completely new agents using molecular modeling, known substrate analogs can also be refined using the data of these aspects of the invention, to provide “second generation” ICL inhibitors with improved therapeutic profiles.

The present structural data are particularly important as they pertain both to ICL from a known pathogen and to complexes of ICL with inhibitors. These data further overcome the drawbacks with other structural studies, such as the difficulties in obtaining ordered crystals of the native ICL enzyme from E. coli (Abeysinghe et al., 1991) and the limitations of the Aspergillus nidulans apo ICL study reported by Britton et al (2000). As the active site region in the Britton et al. (2000) structures was disordered, several important questions were left unanswered, severely limiting the usefulness of such data in drug design. In contrast, the present invention solves the structure of native ICL from M. tuberculosis using the multiwavelength anomalous dispersion (MAD) method (Table 1) and provides useful structures of ICL after reaction with 3-bromopyruvate and of the C191S mutant in complex with 3-nitropropionate and glyoxylate. This information can now be used in rational drug design.

Moreover, as operation of the glyoxylate shunt is a key feature amongst other microbes that give rise to persistence infections, the present invention is not limited to therapeutic intervention in Mycobacteria. Indeed, a wide range of organisms can be targeted by the present invention, including Pseudomonas, Salmonella, Yersinia, and Leishmania, all of which cause persistent infection in humans.

One important aspect of the invention is the design of inhibitors for use in treating fungal infections, such as those caused by C. albicans and other pathogenic fungi (Example 4). Fungal infections constitute a significant worldwide health problem. In addition to life-threatening systemic infections in immunocompromised individuals, low grade fungal infections also pose a meaningful health risk. Commonly known consequences of fungal infections include athletes foot, scalp itch, jock itch, ringworm and other cutaneous mycoses; sinusitis; thrush and vaginal candidiasis; nasal poly formation, asthma and eosinophilias. In addition, chronic low grade fungal infections in otherwise healthy human subjects are likely causative agents of tissue allergic responses.

The anti-fungal agents currently available for veterinary and human use suffer from many drawbacks, including toxic side-effects. The poor tissue penetration of many such agents also limits their effectiveness against deep seated fungal infections. Irrespective of the toxicity issues, the available anti-fungal therapeutics are often not fungicidal at the concentrations achievable clinically and only prove to be “fungistatic”. Moreover, the ability of pathogenic fungi to evade both host defenses and clinical intervention is shown by the fact that many individuals often have difficulty in completely eliminating fungal infections. The application of the present invention in the development of improved anti-fungal agents is therefore a significant advance.

As the glyoxylate shunt enzyme targets of the present invention are present in microbes and plants, but not in animals and humans, the inhibitors identified or designed by application of the present invention are ideal as antibacterial and antifungal agents. The safety profile for such agents is thus a particularly attractive aspect of this invention. This is another feature emphasizing the preference for isocitrate lyase and malate synthase, which do not exist in mammalian cells.

### II. Structural Data and Molecule Modeling

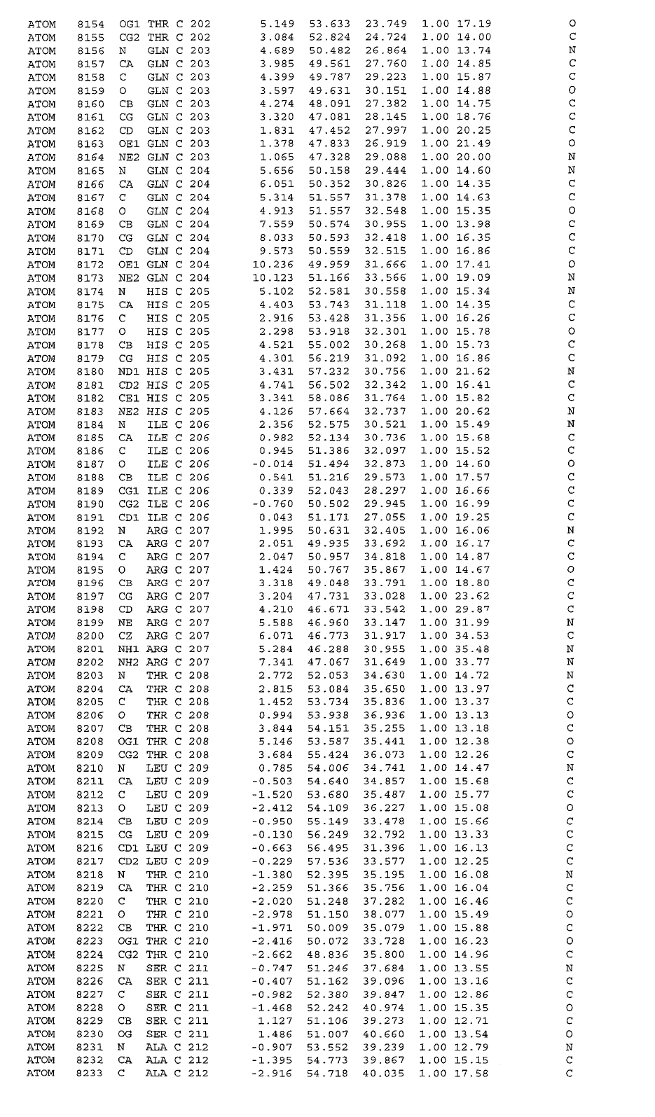

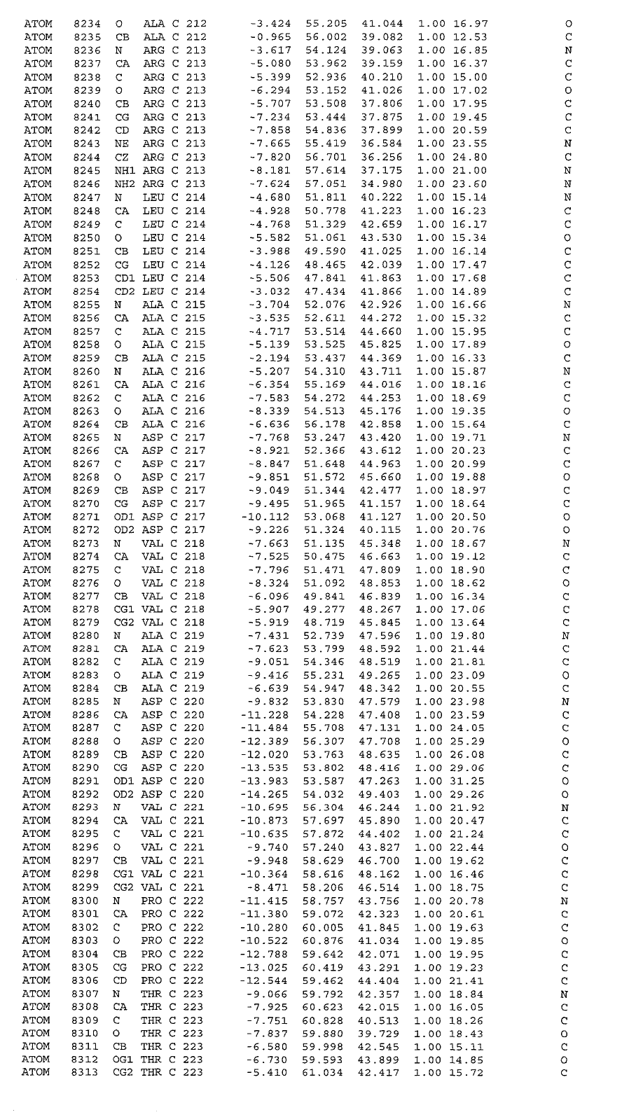

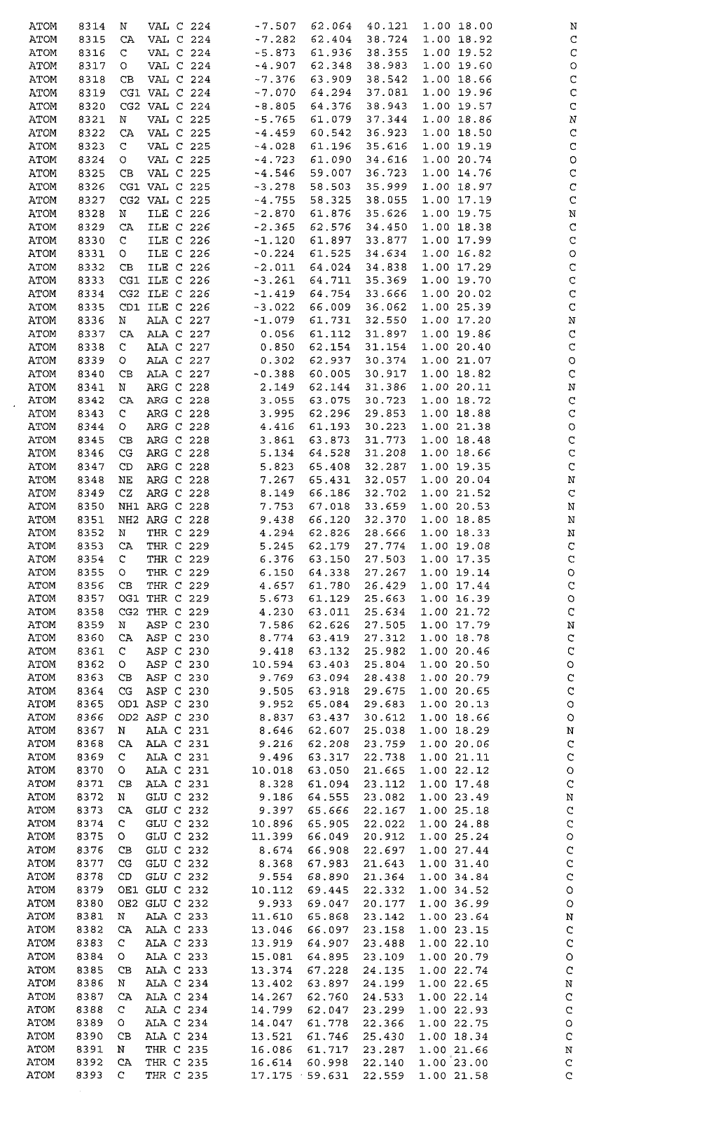

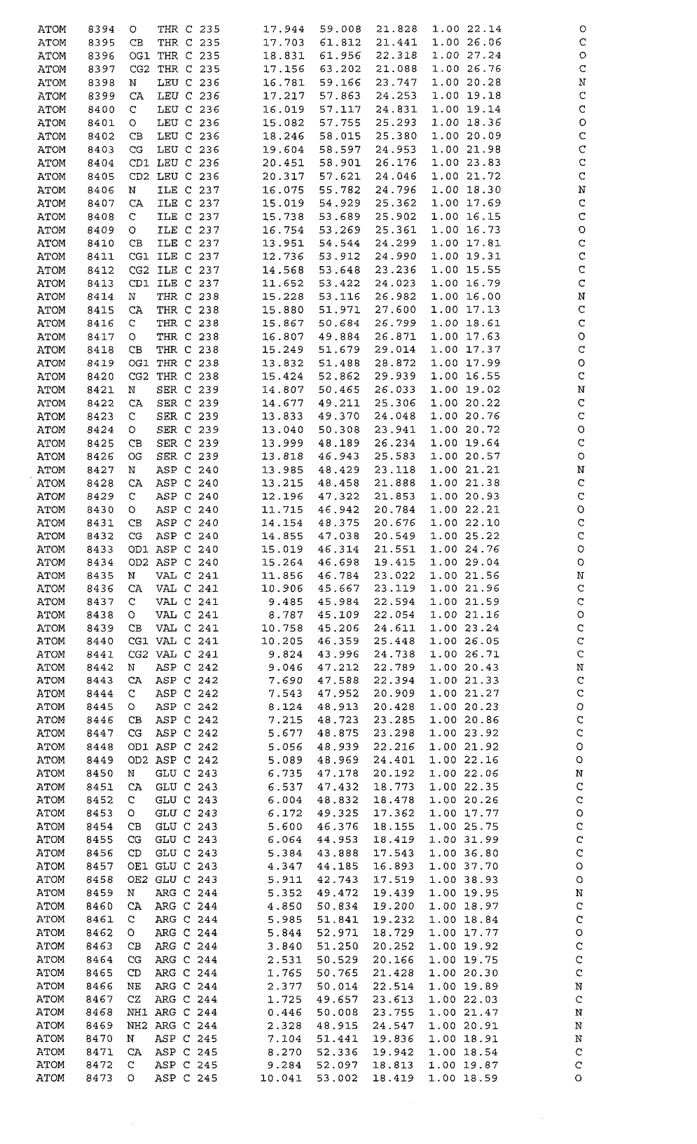

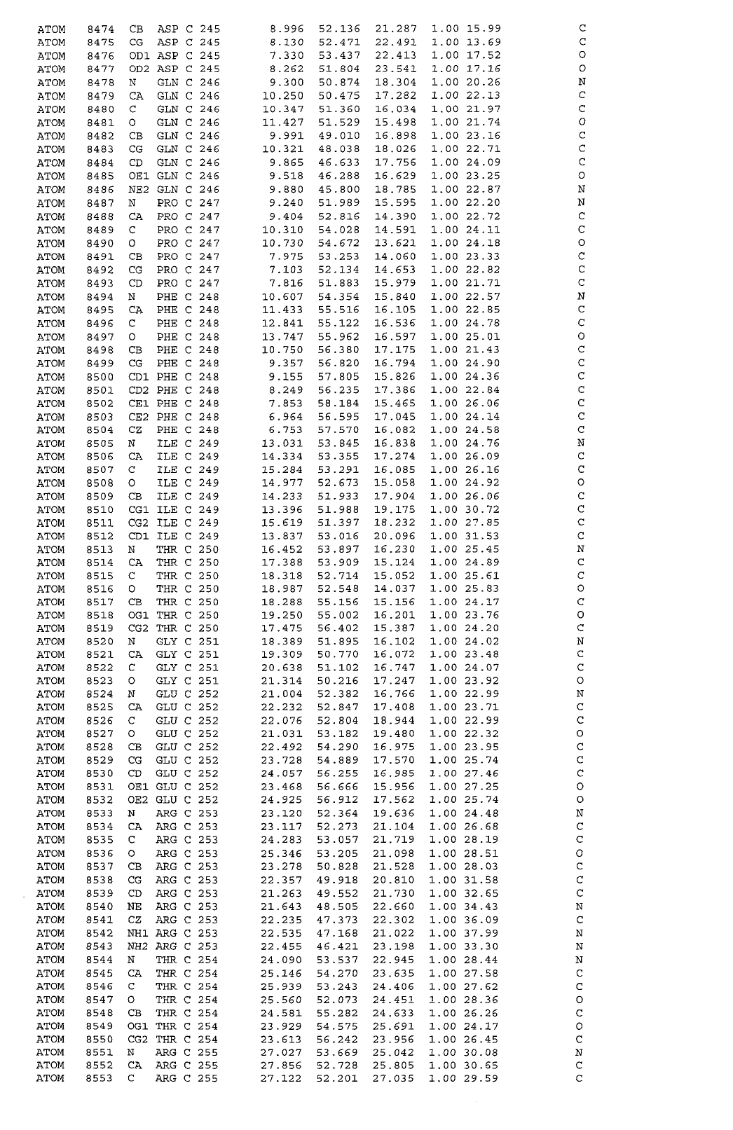

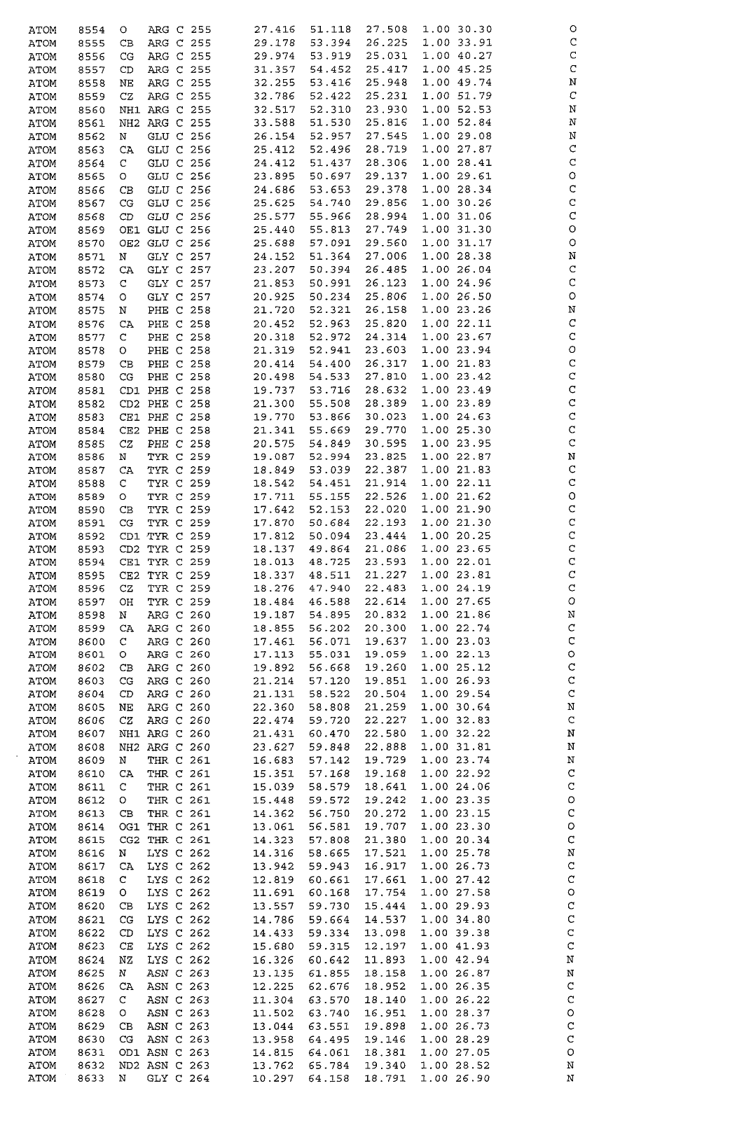

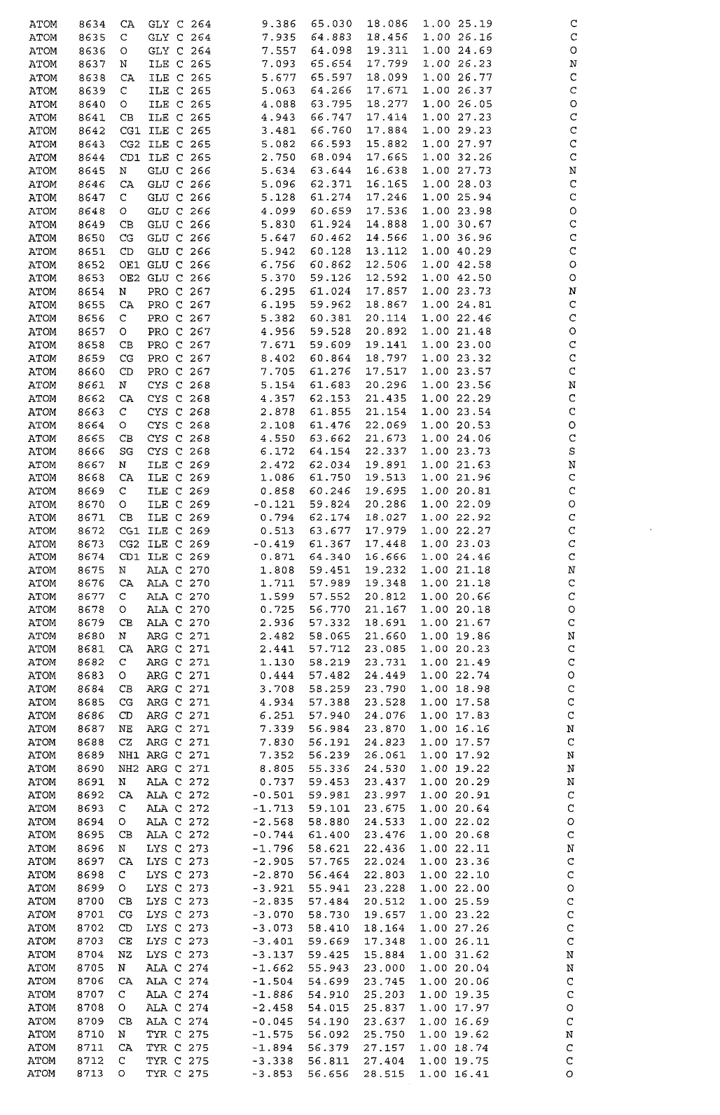

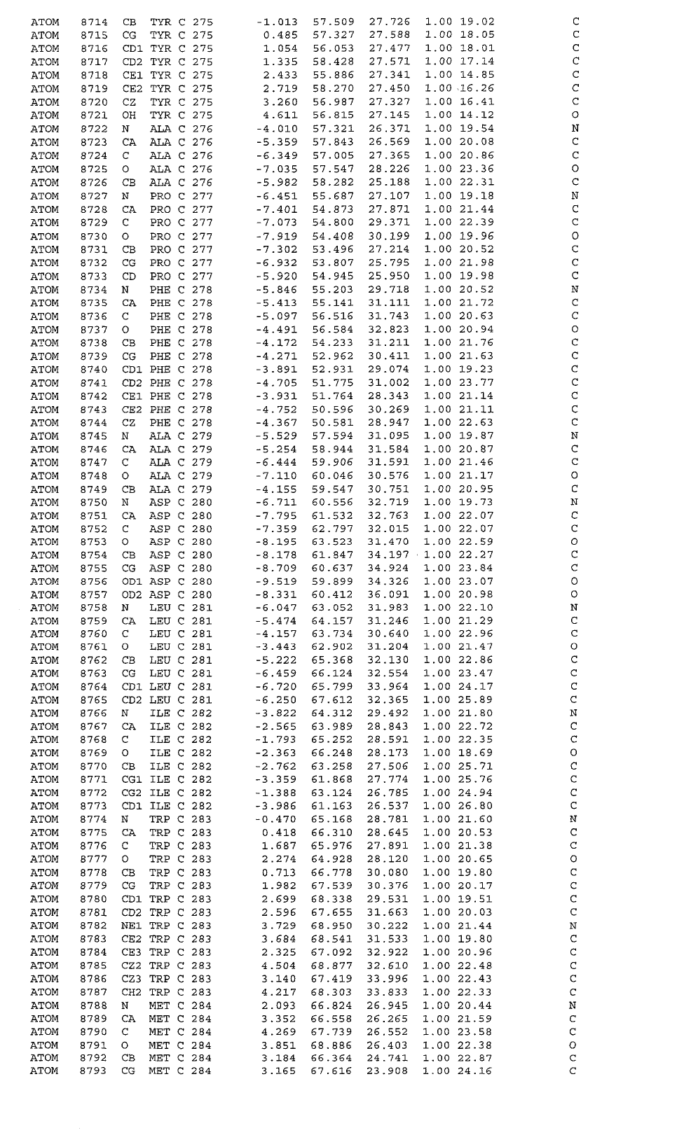

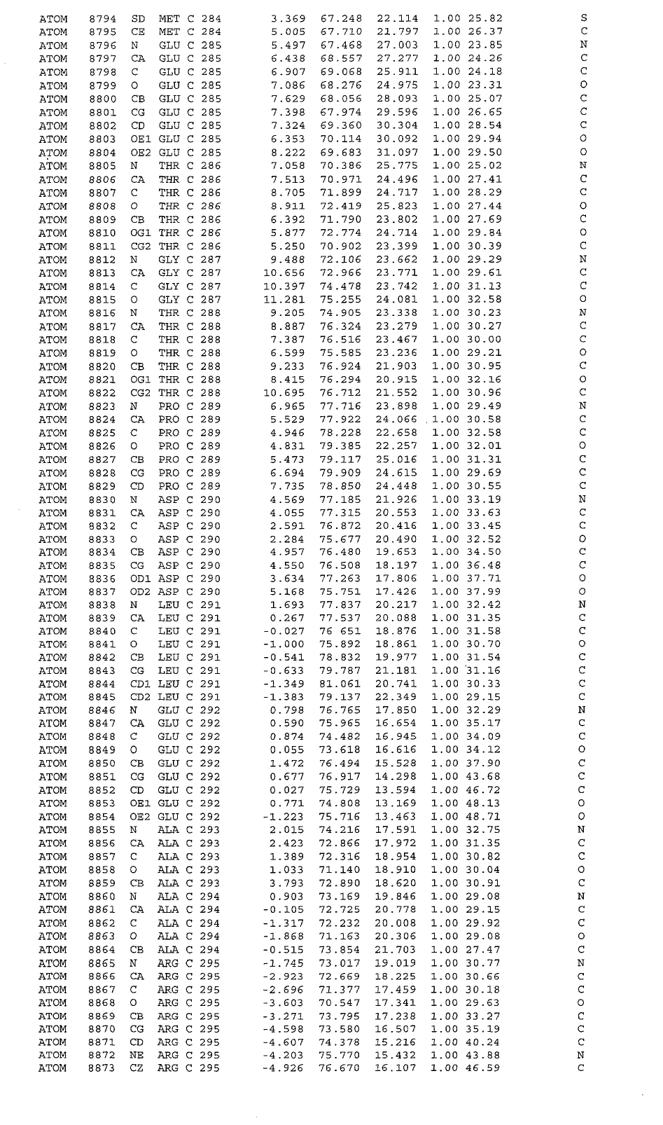

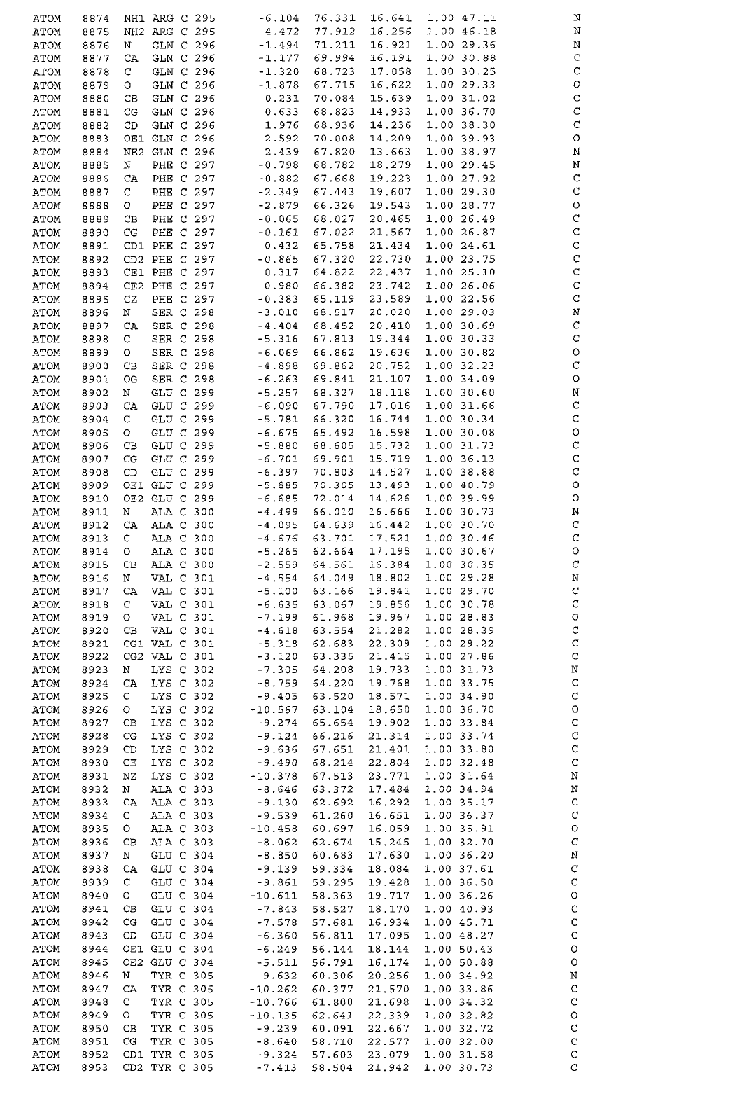

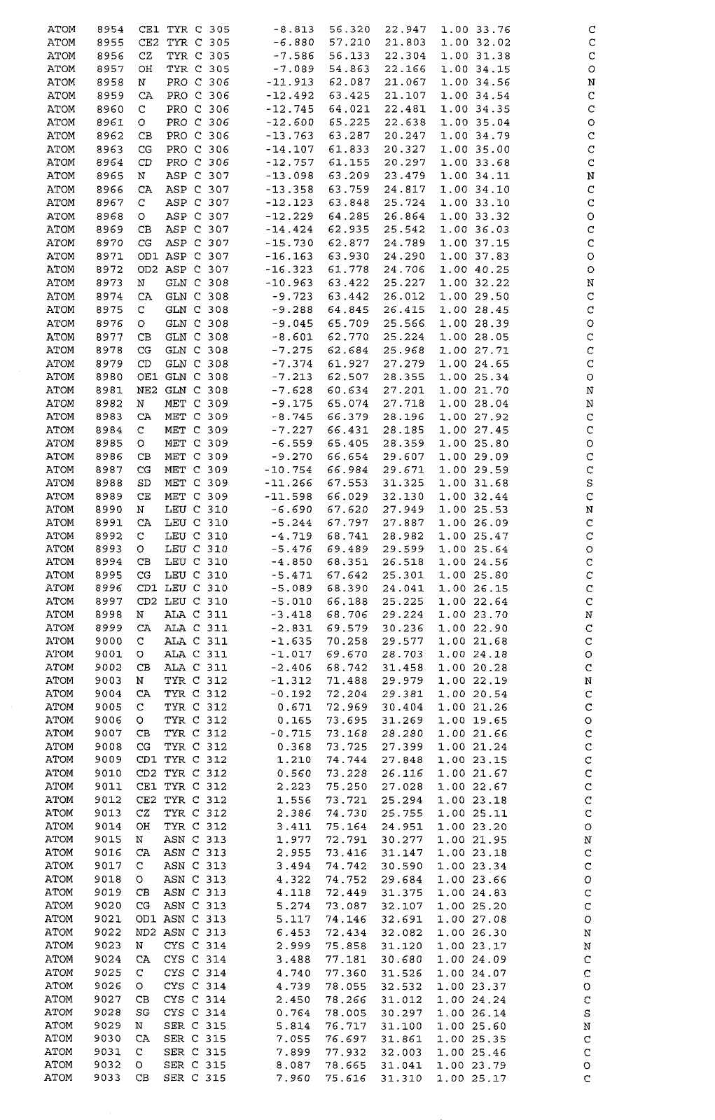

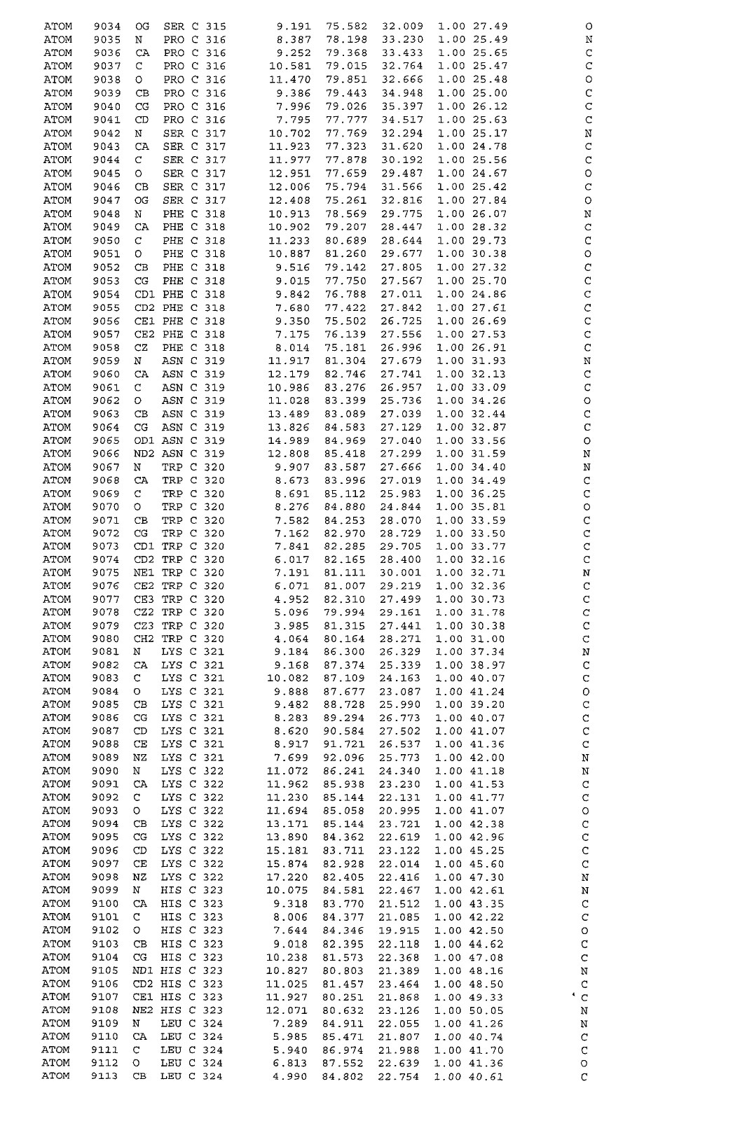

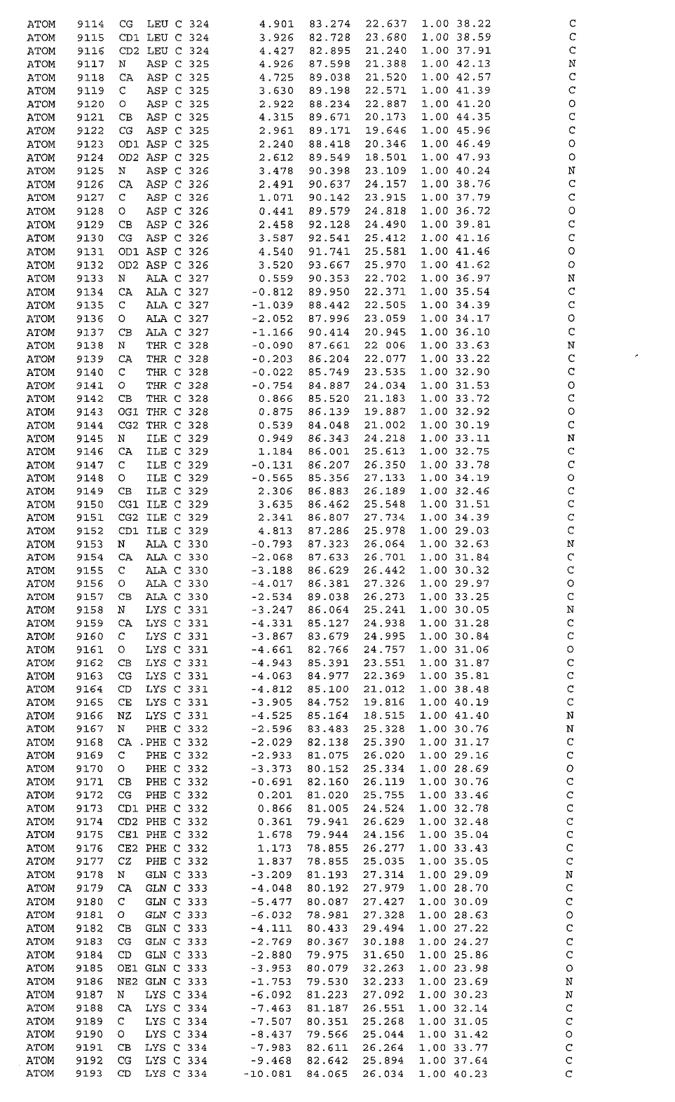

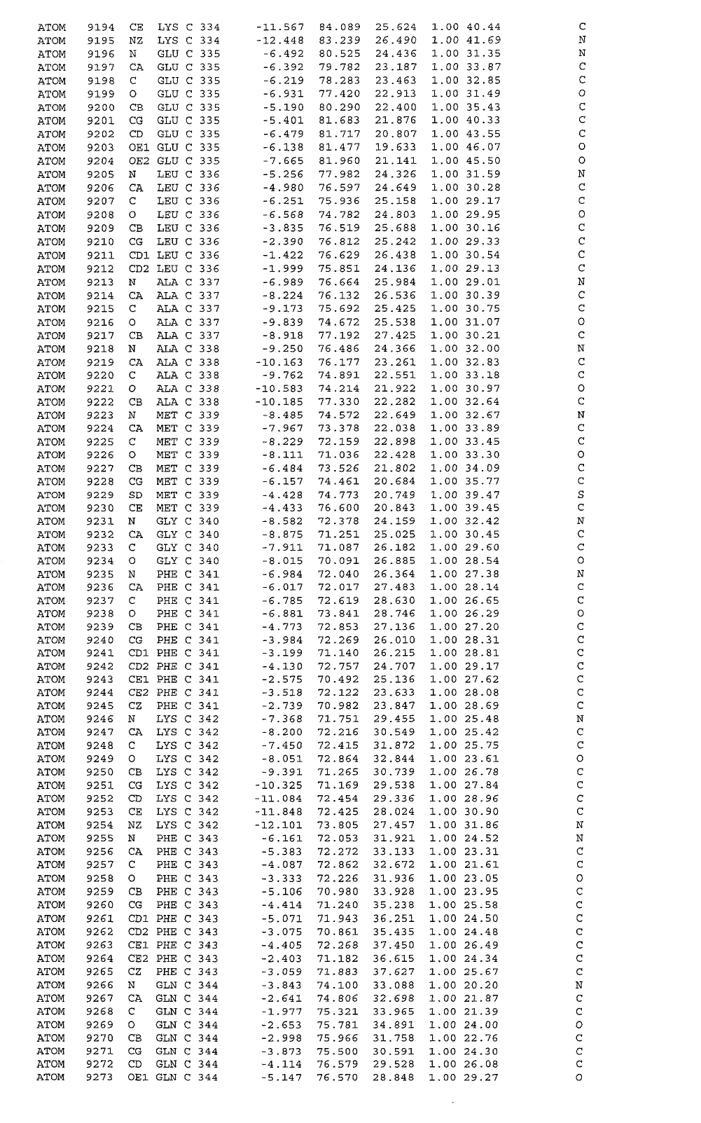

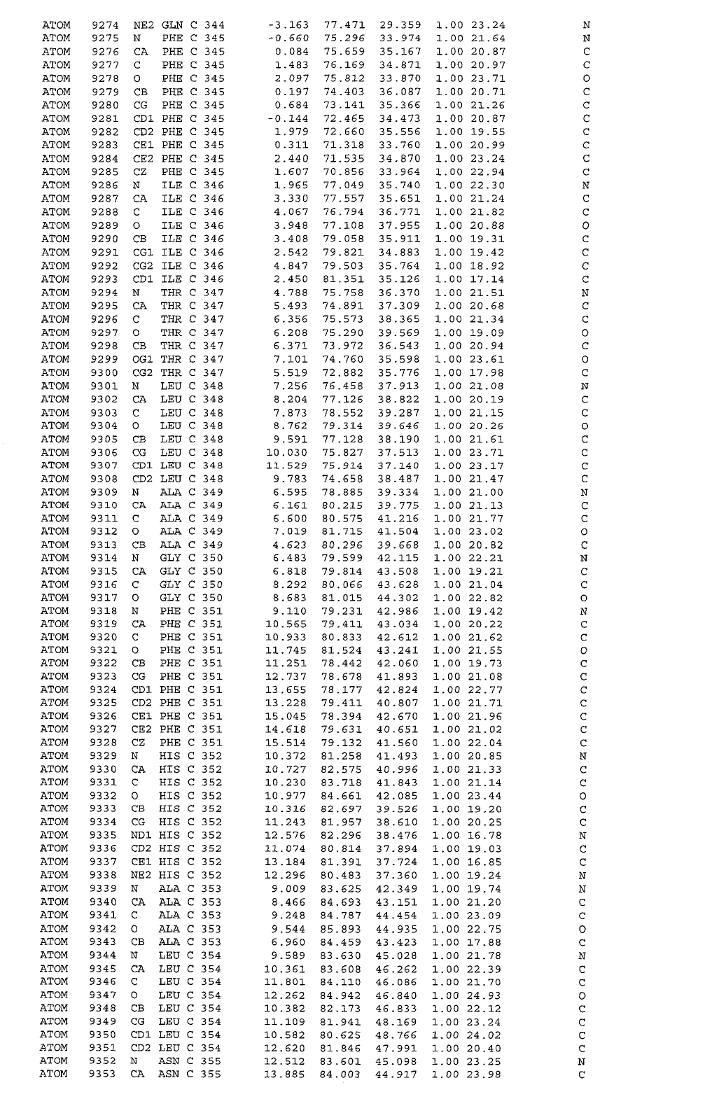

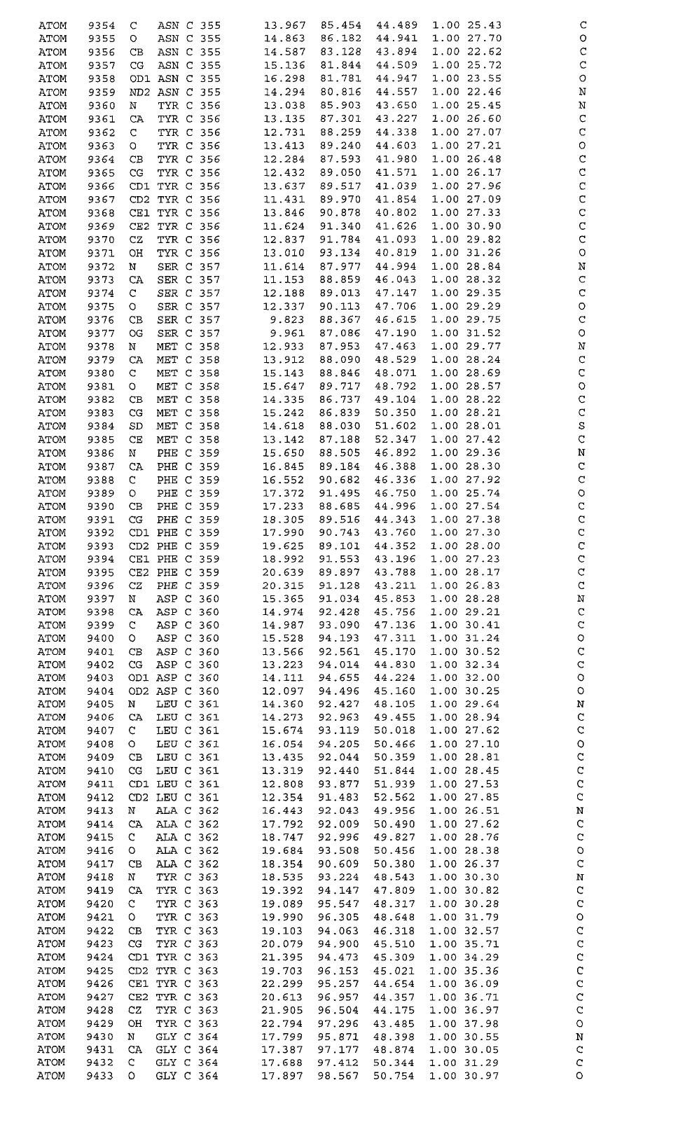

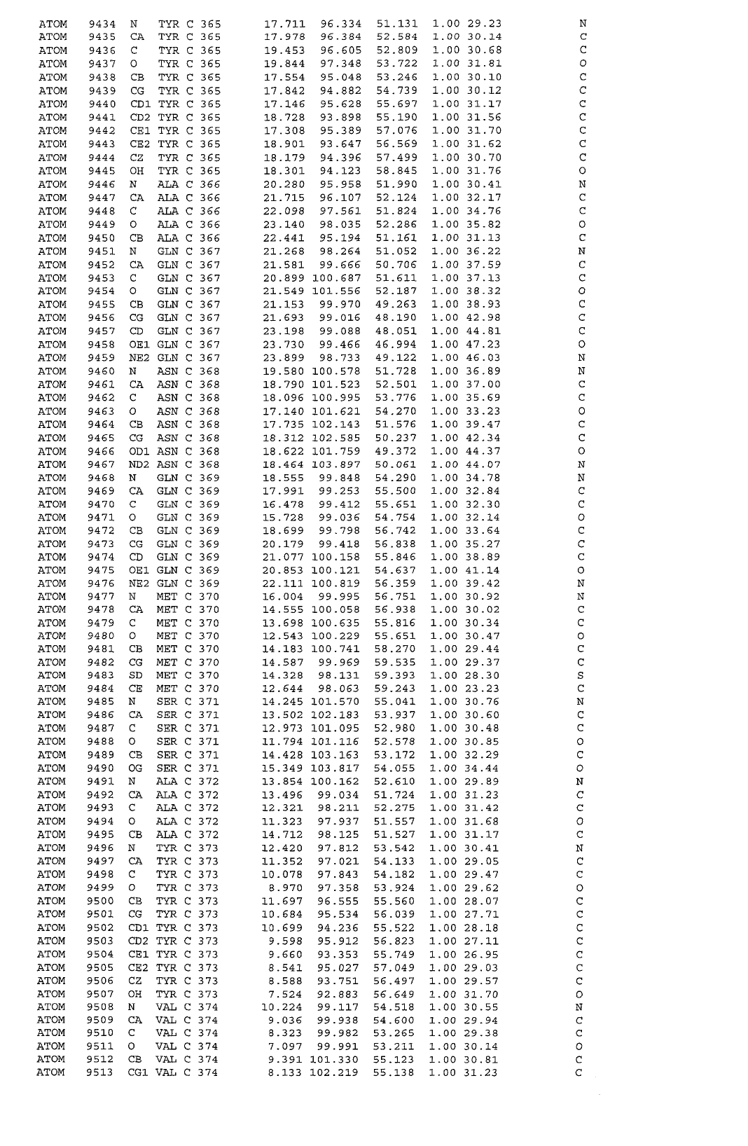

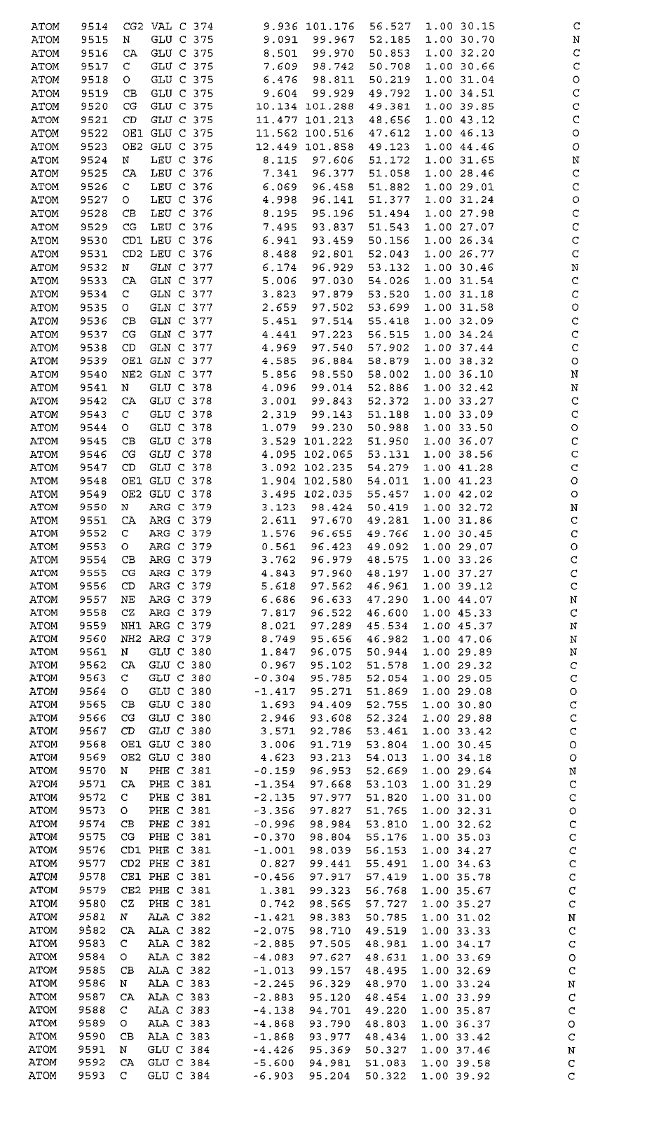

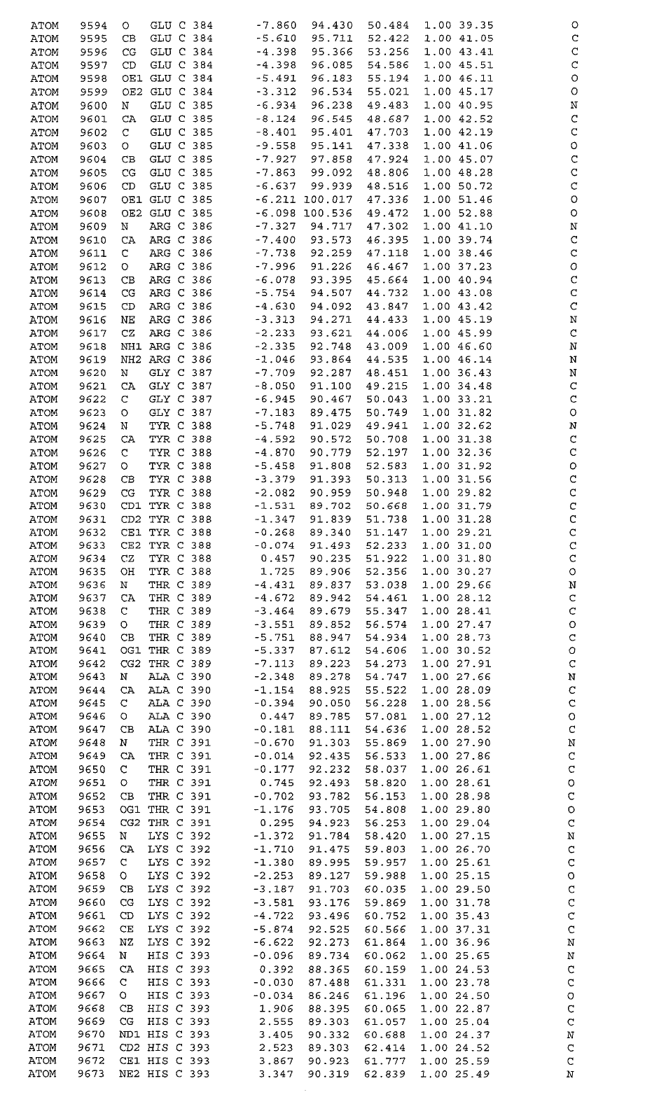

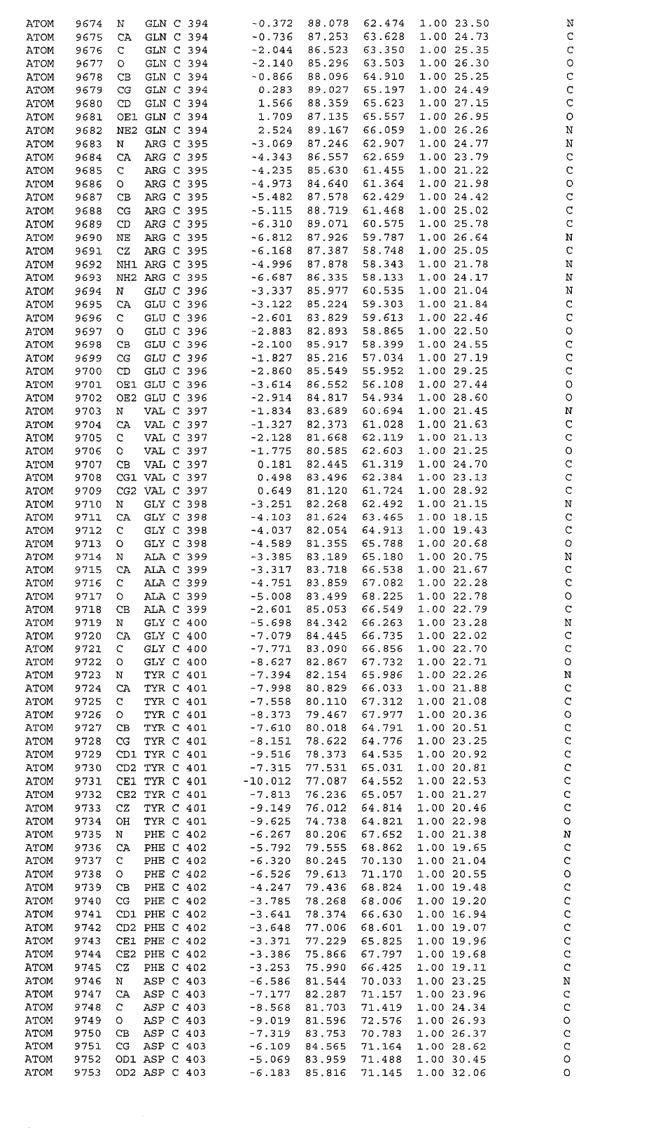

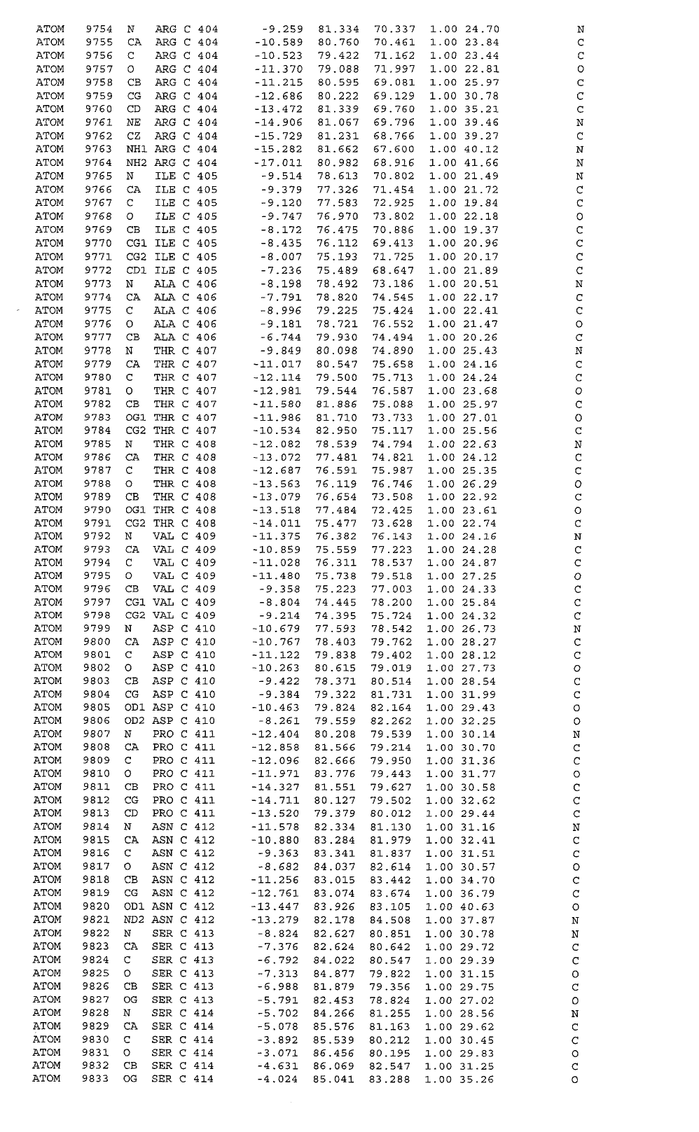

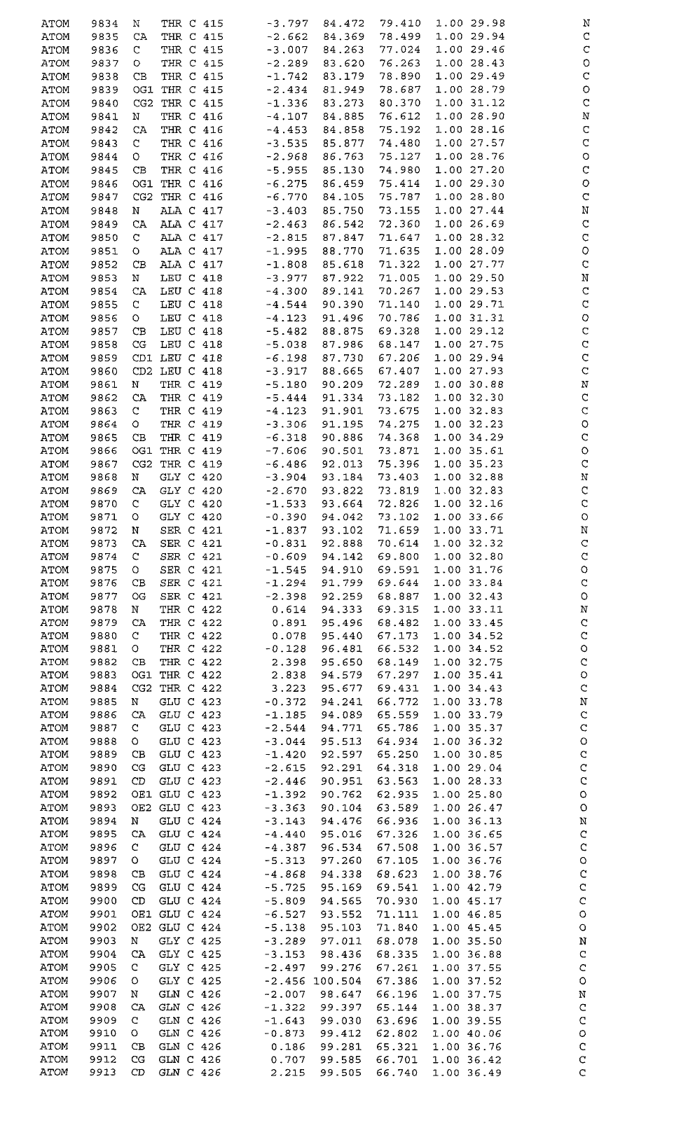

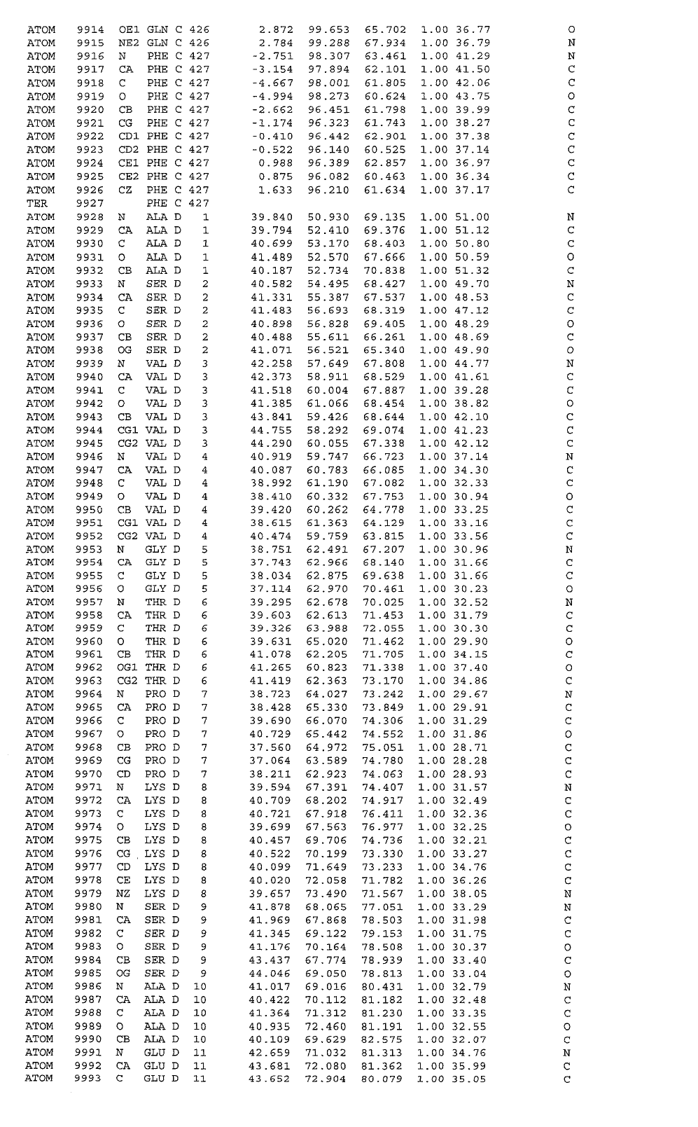

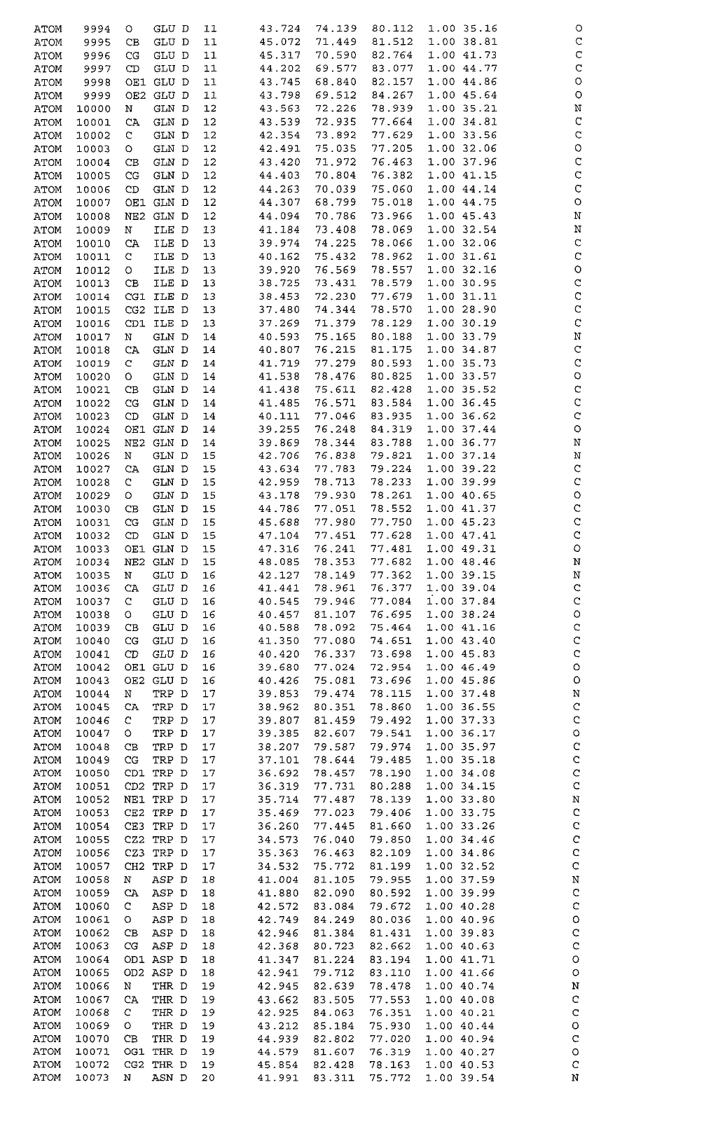

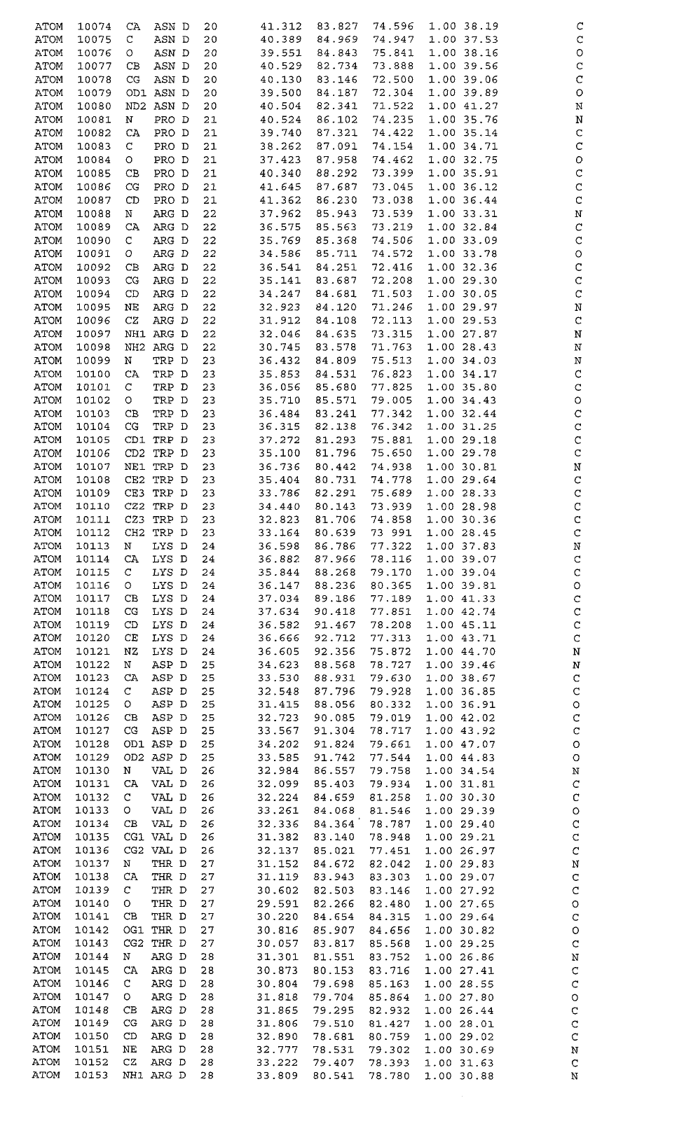

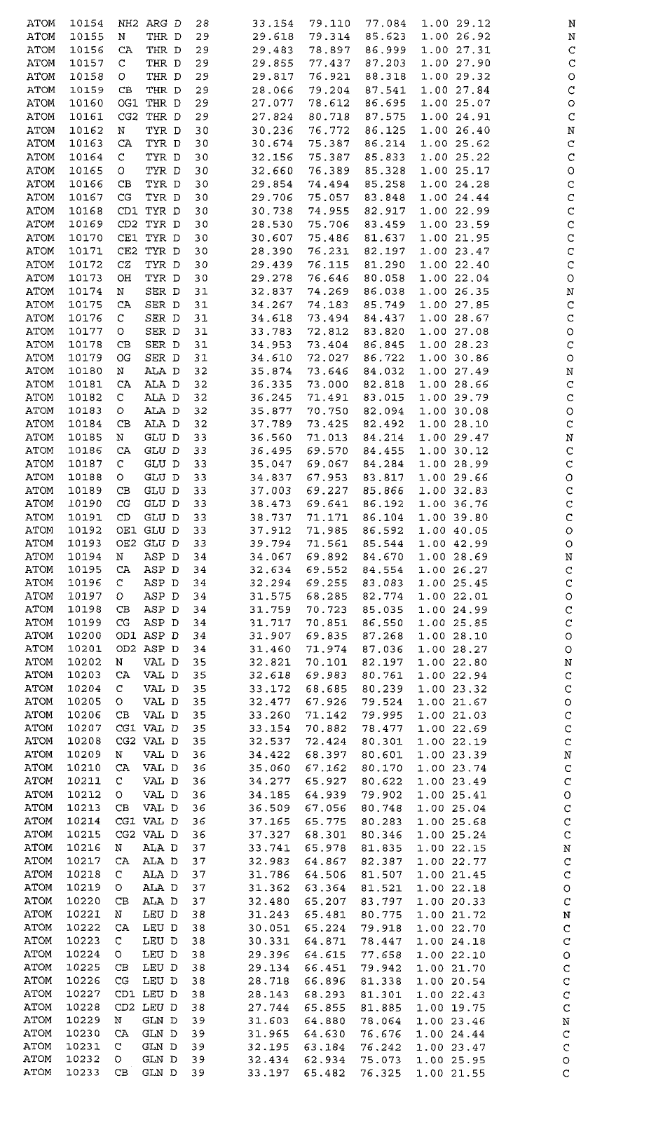

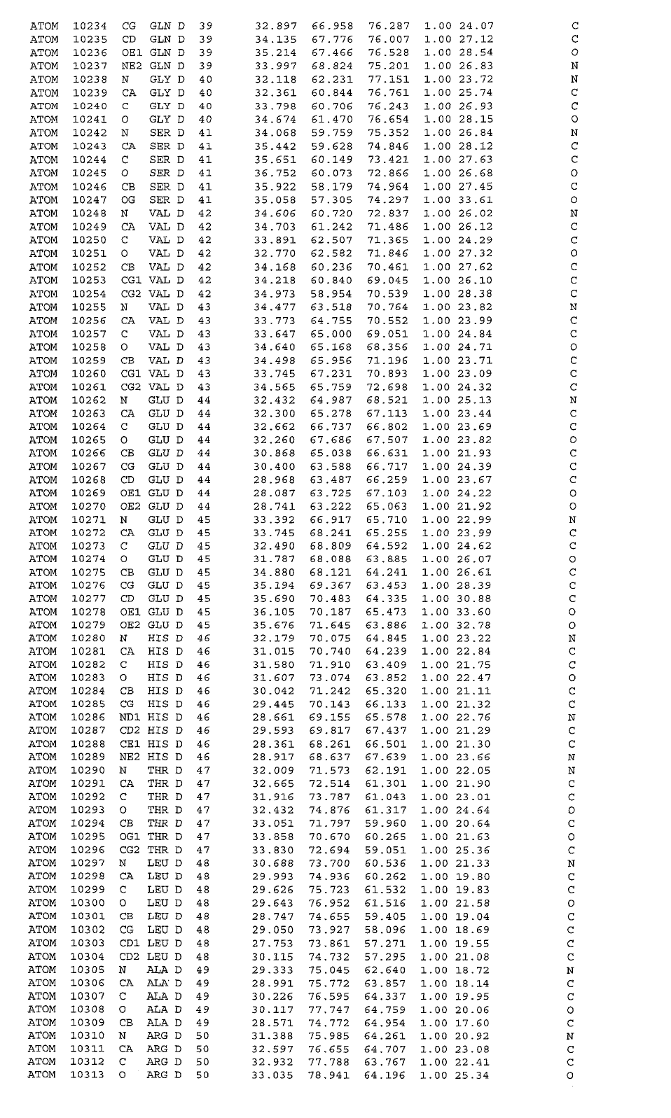

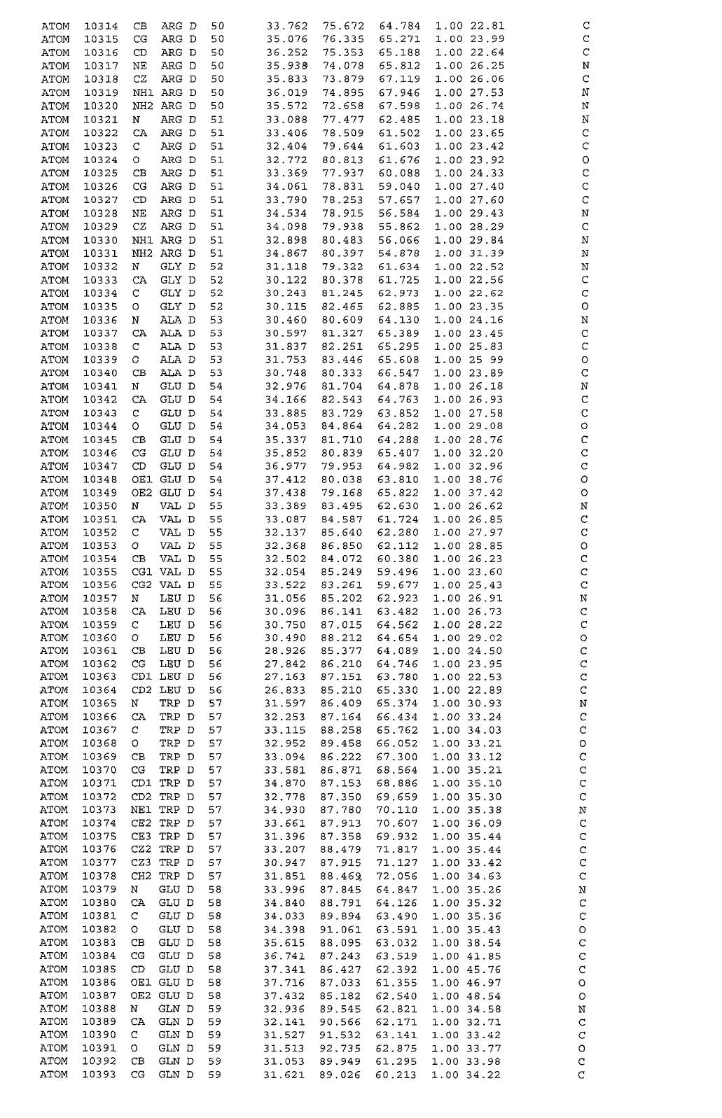

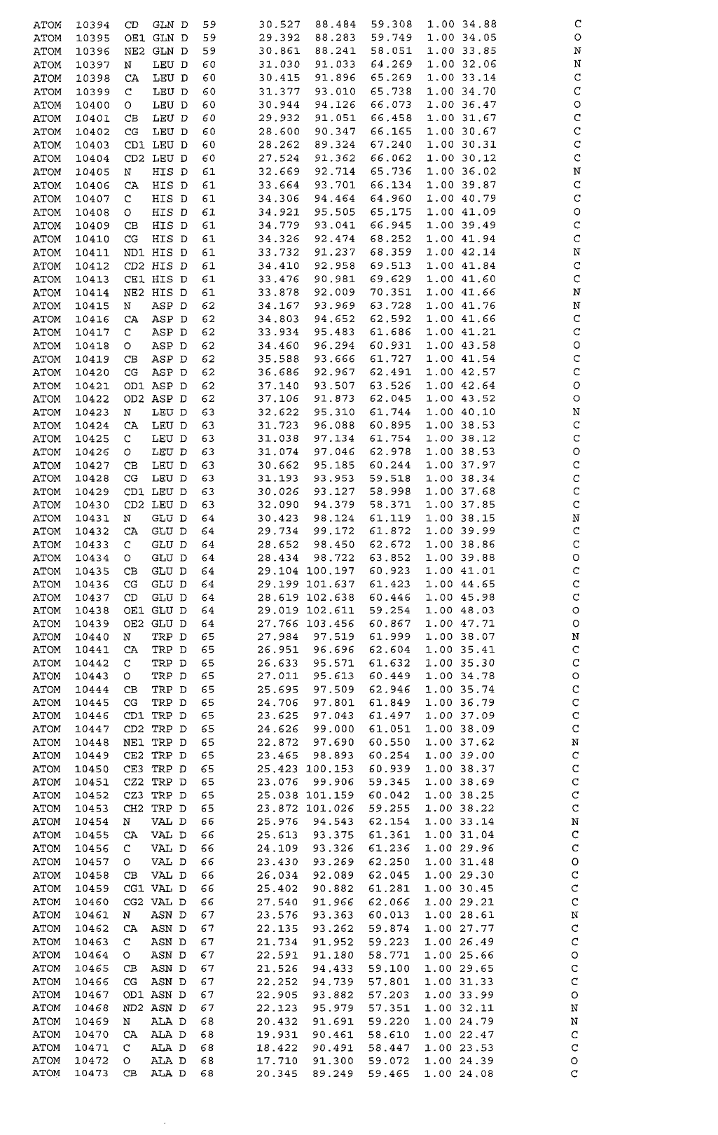

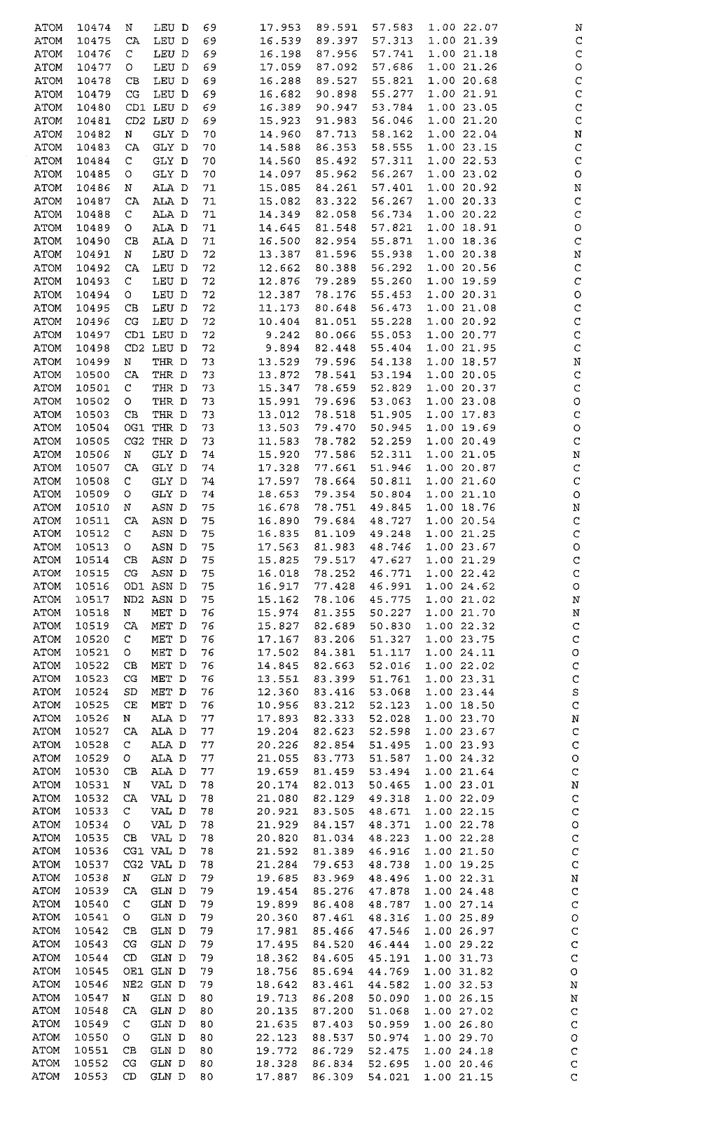

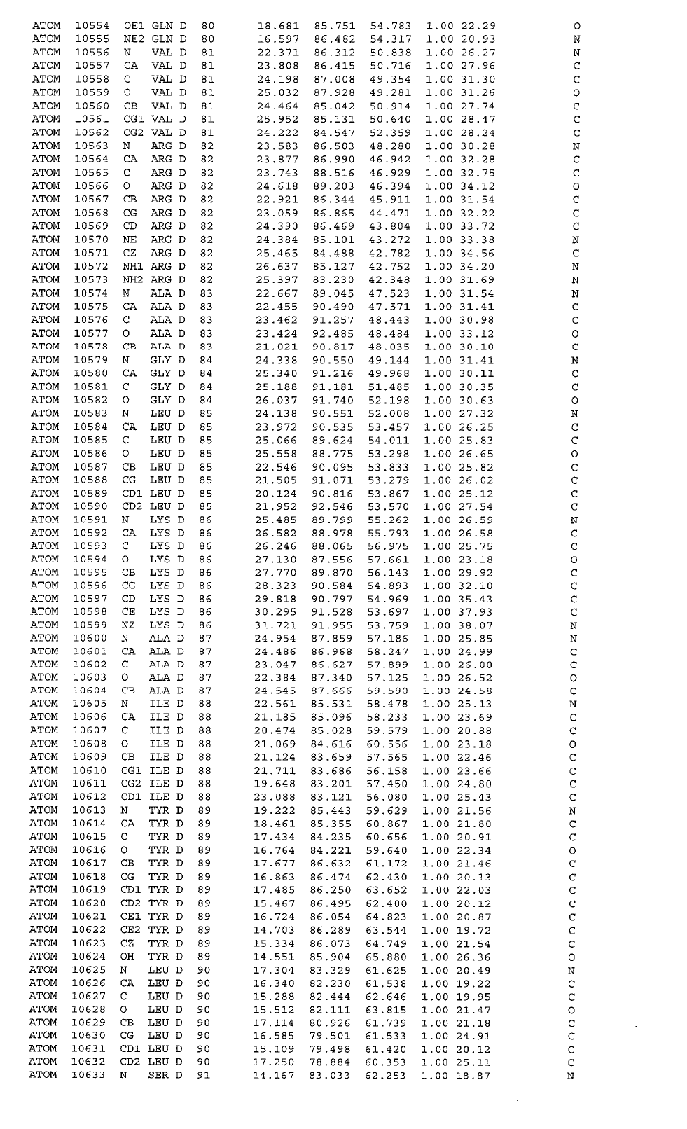

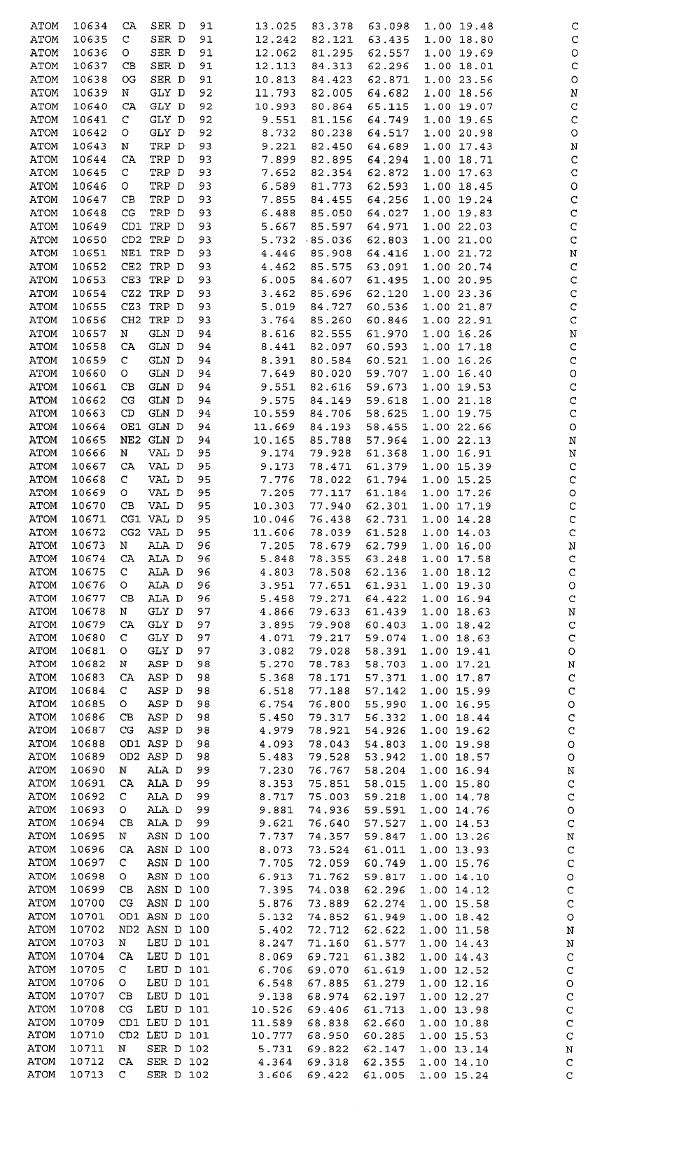

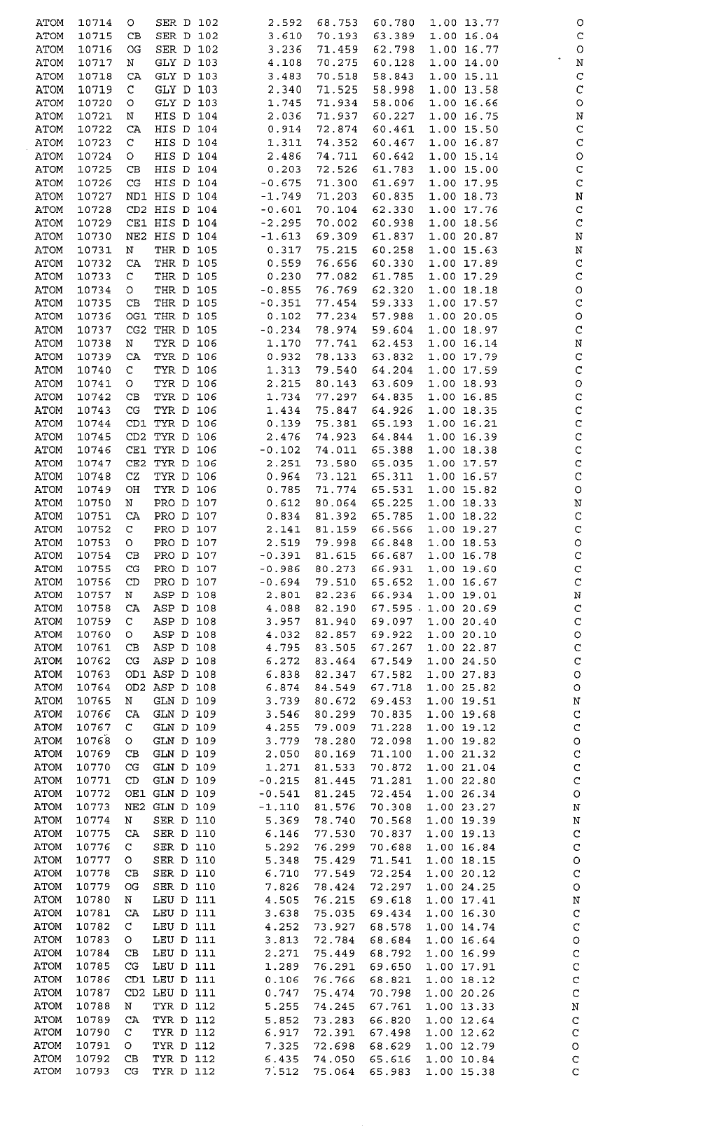

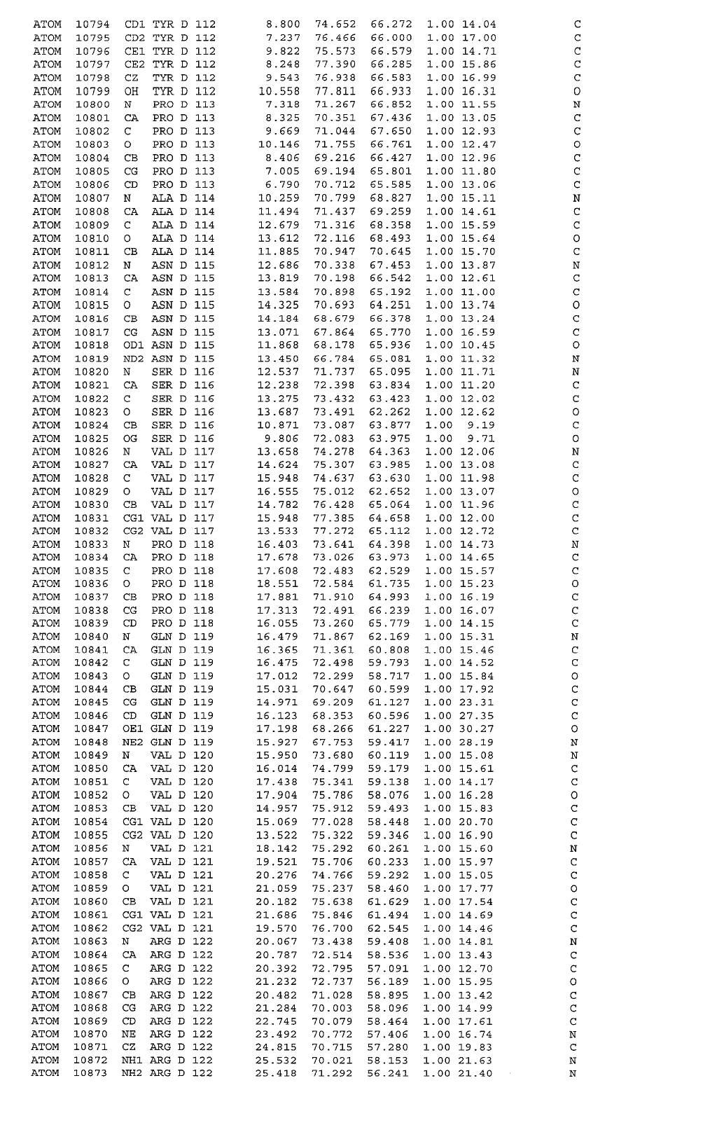

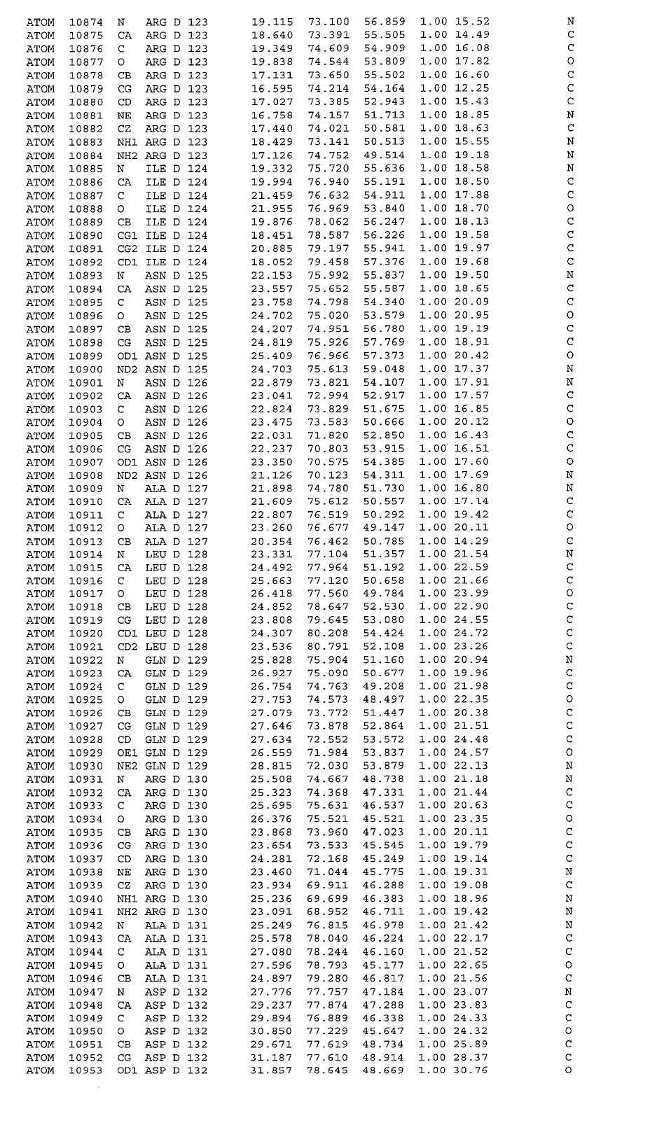

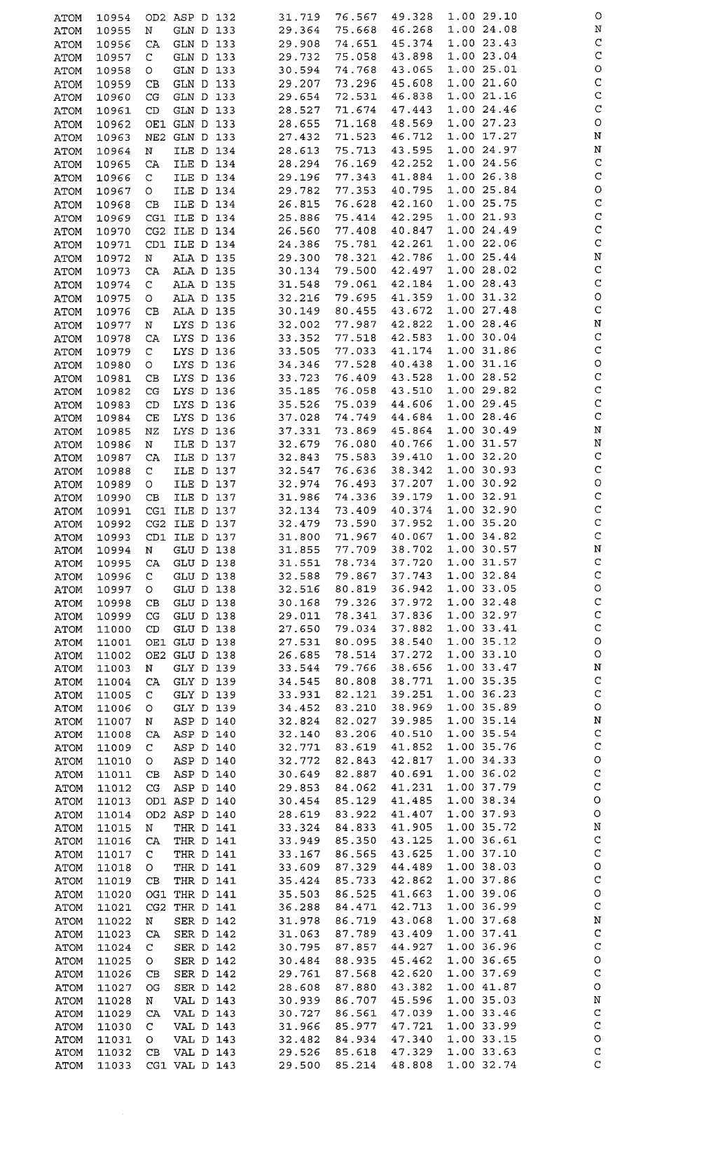

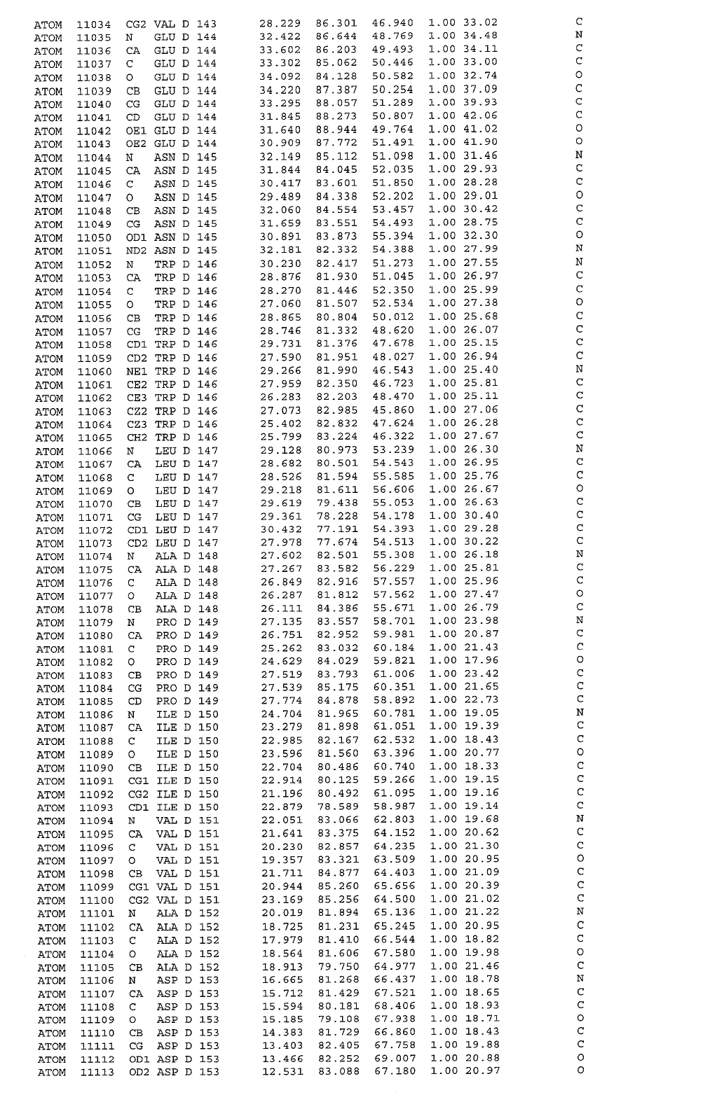

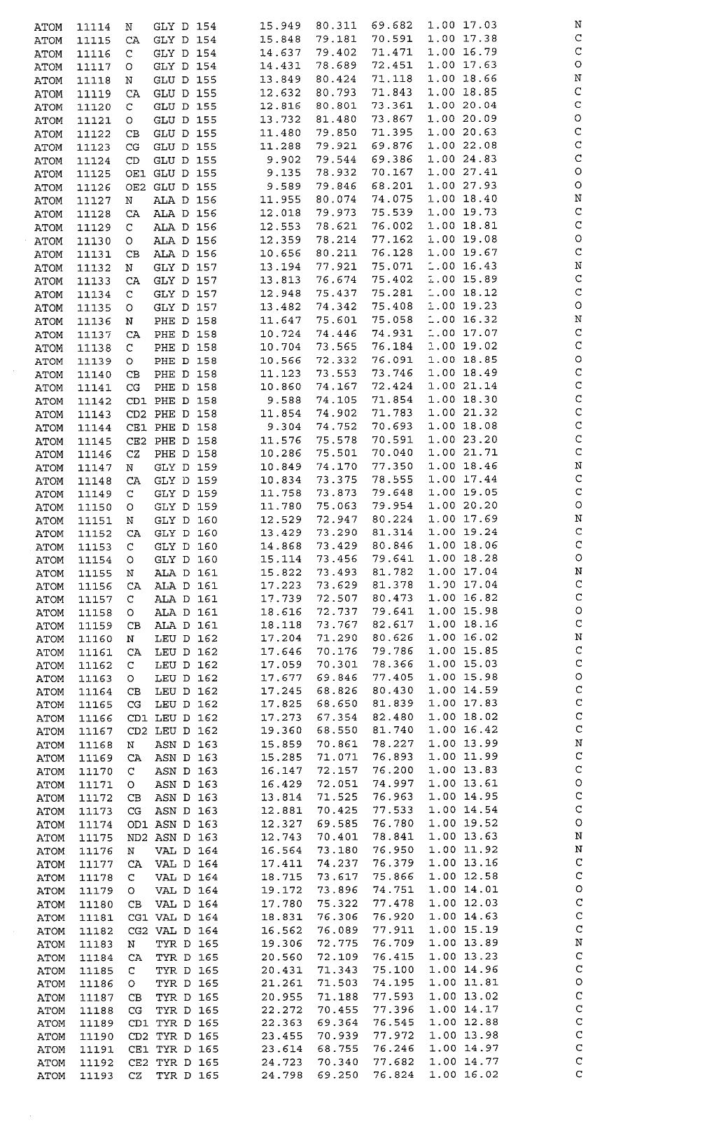

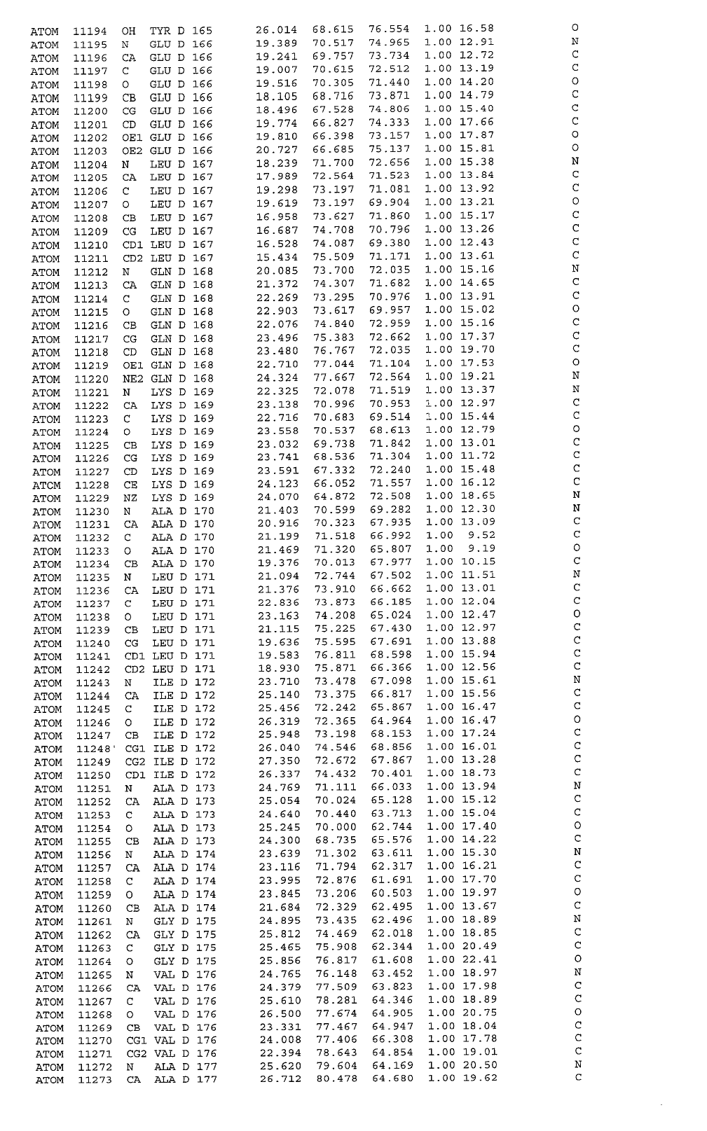

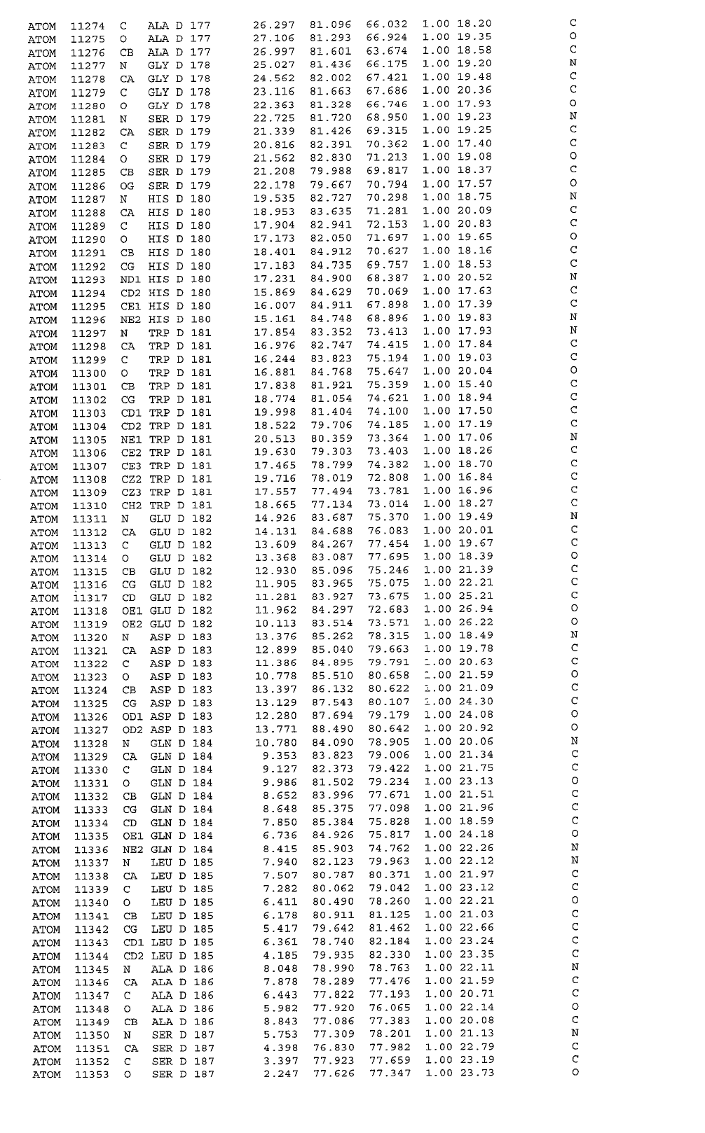

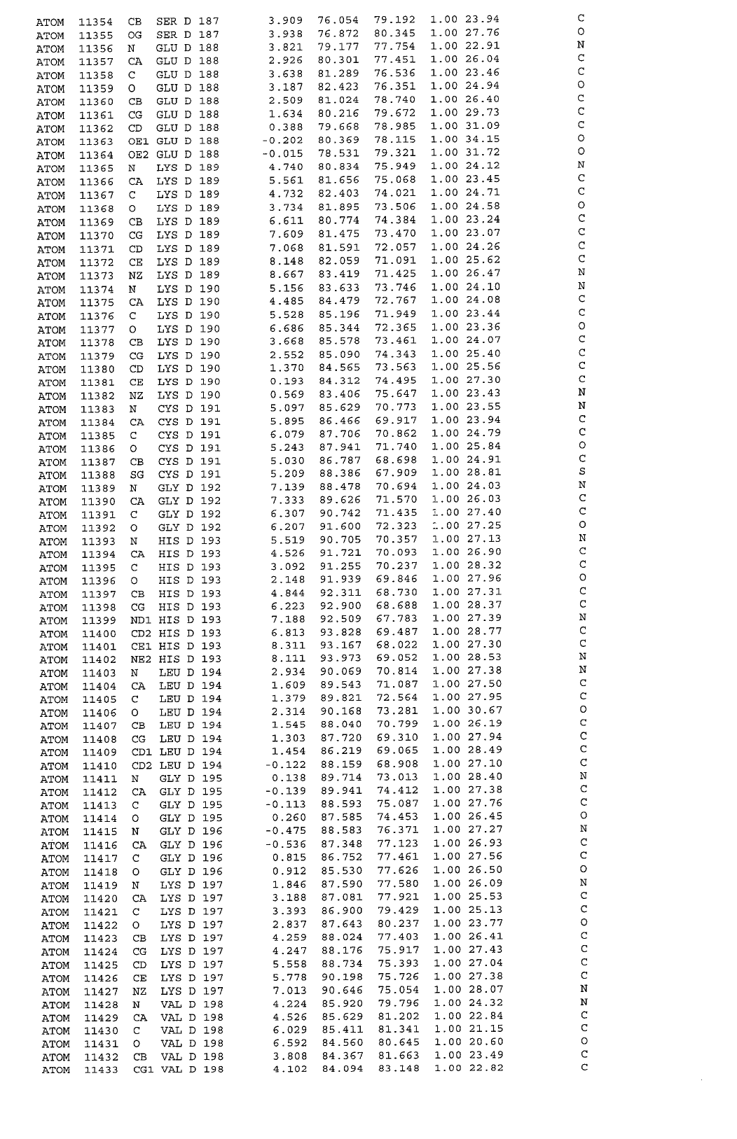

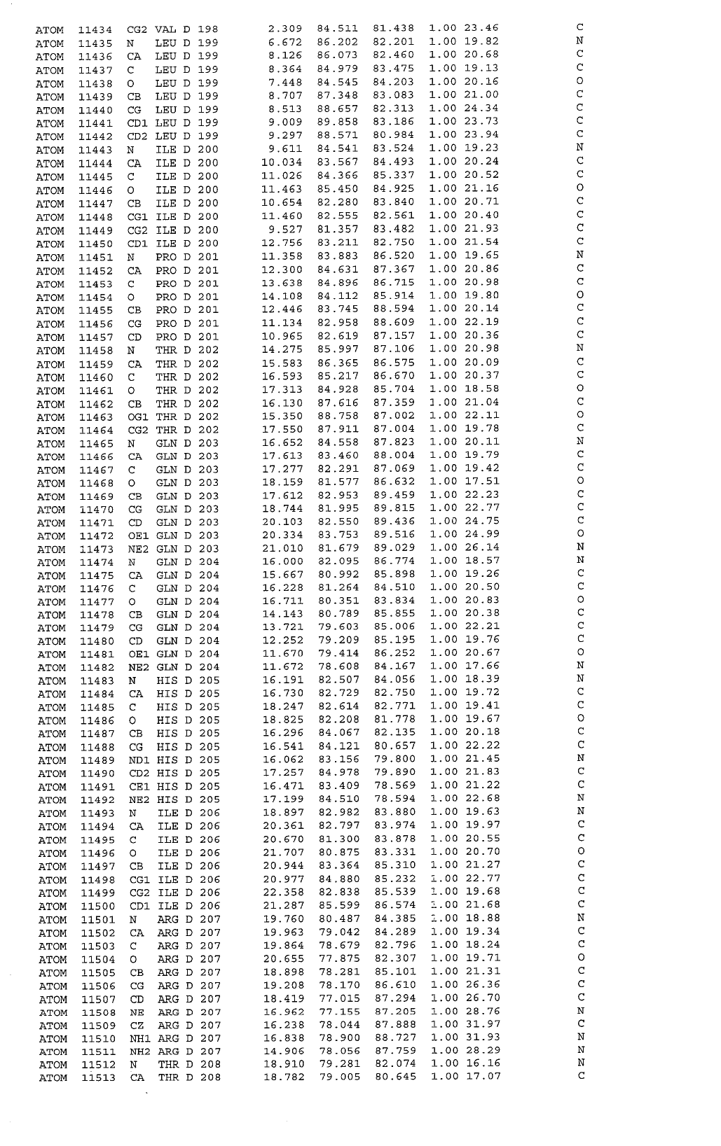

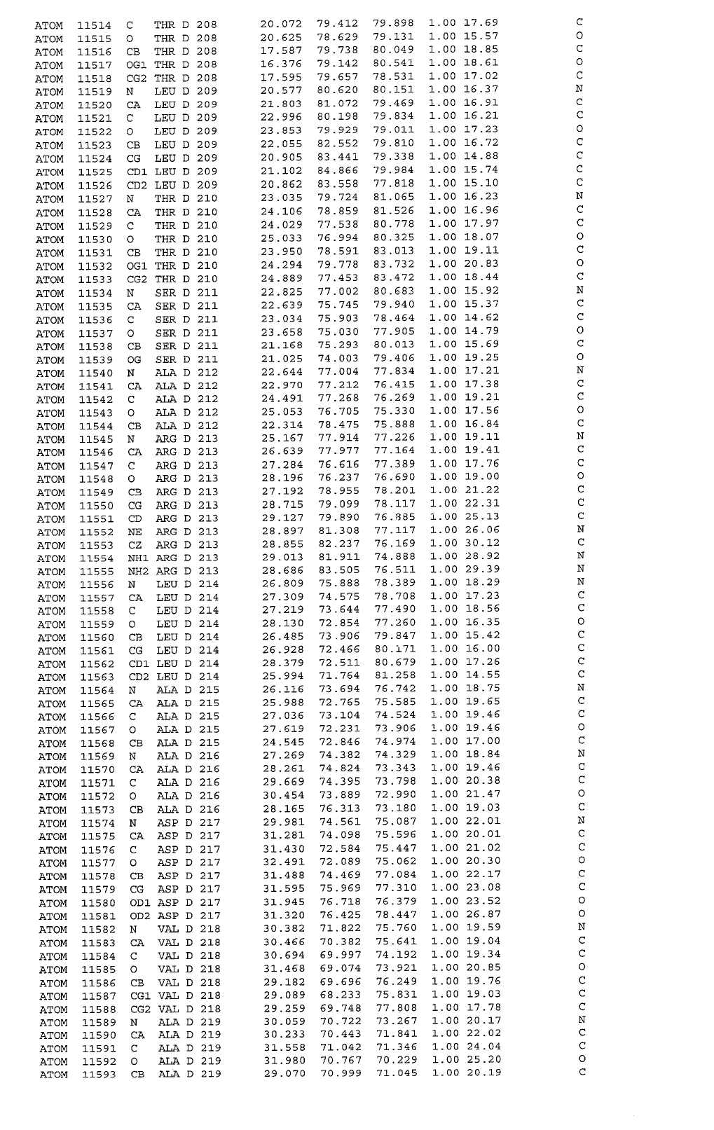

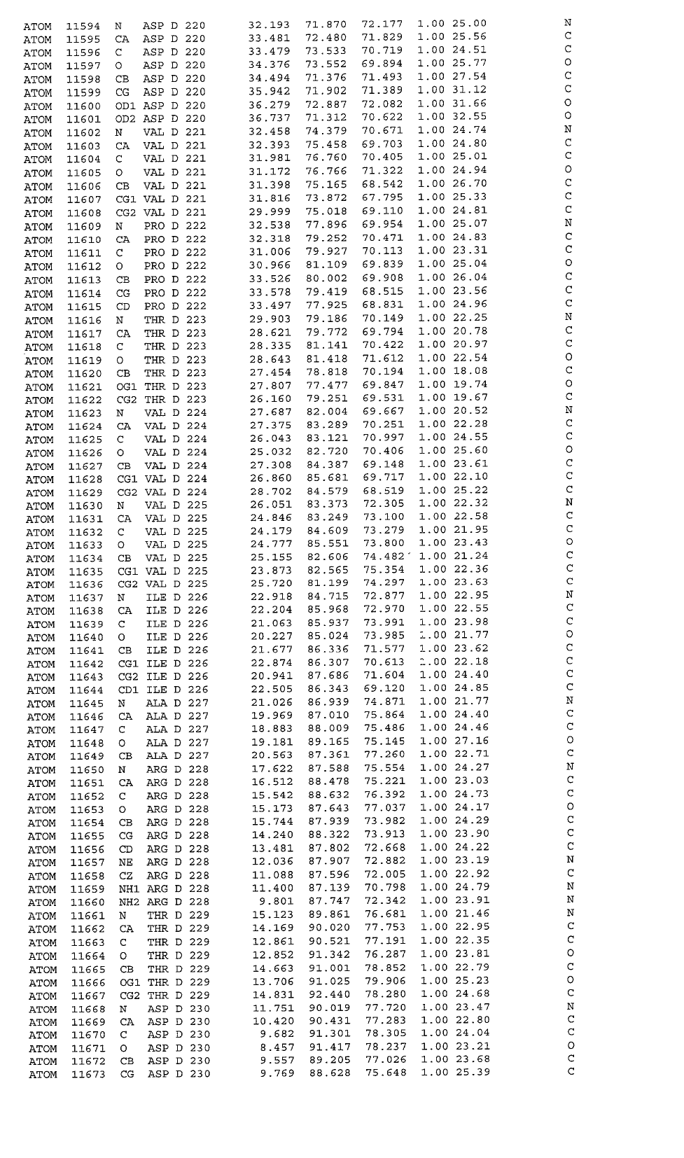

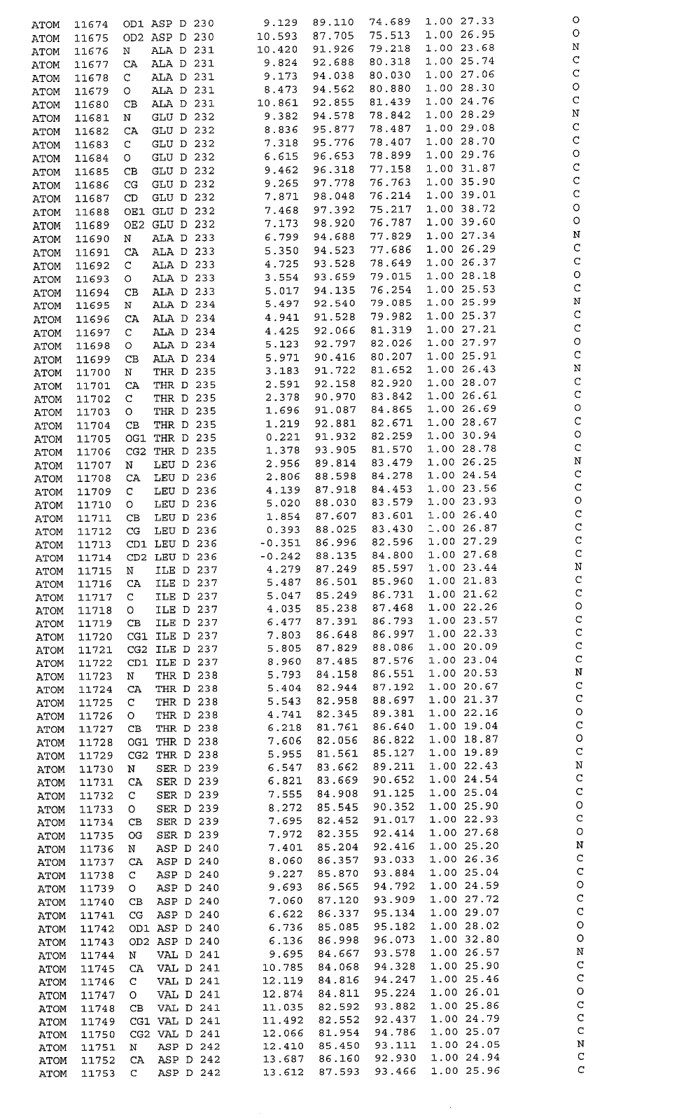

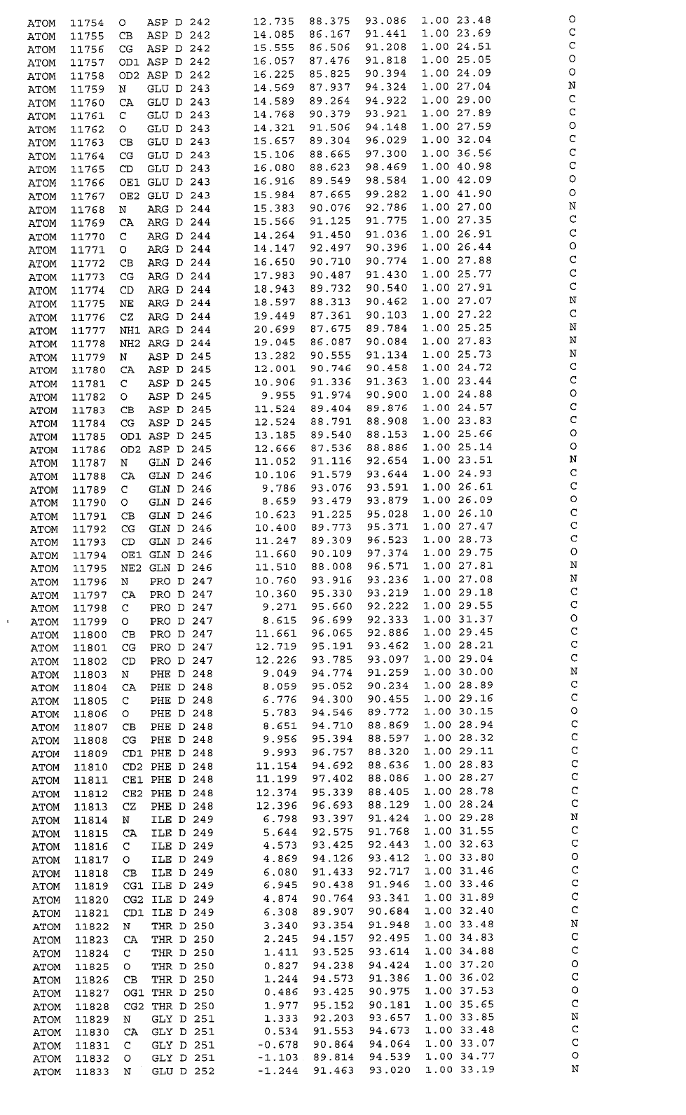

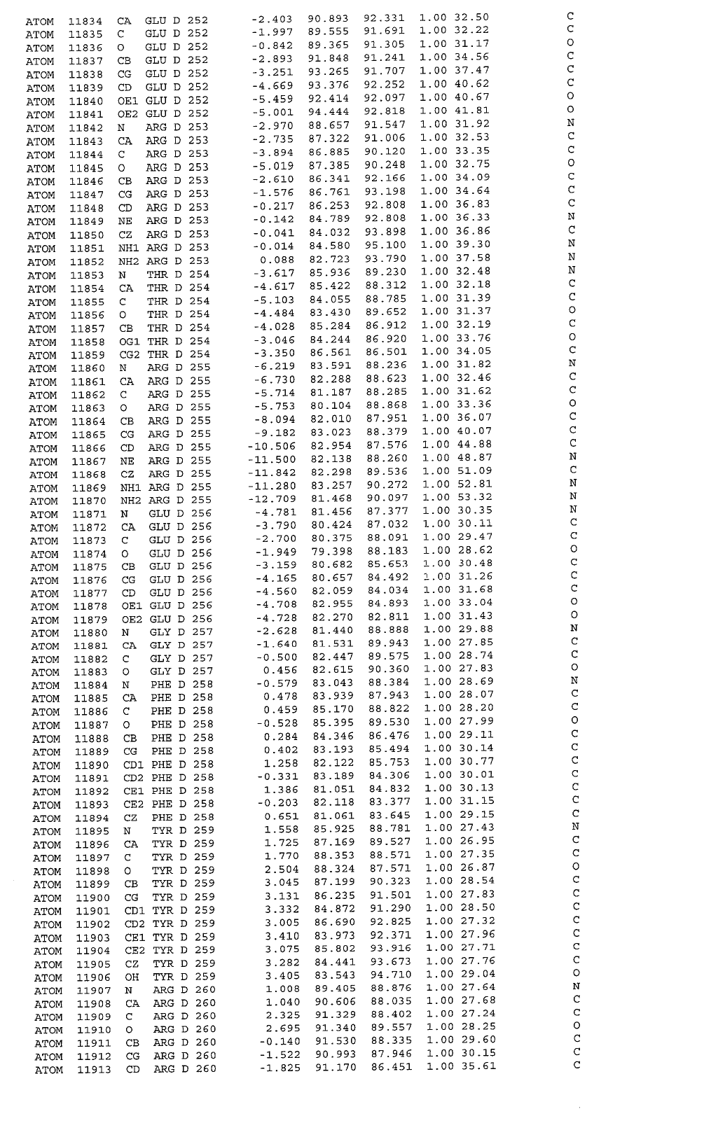

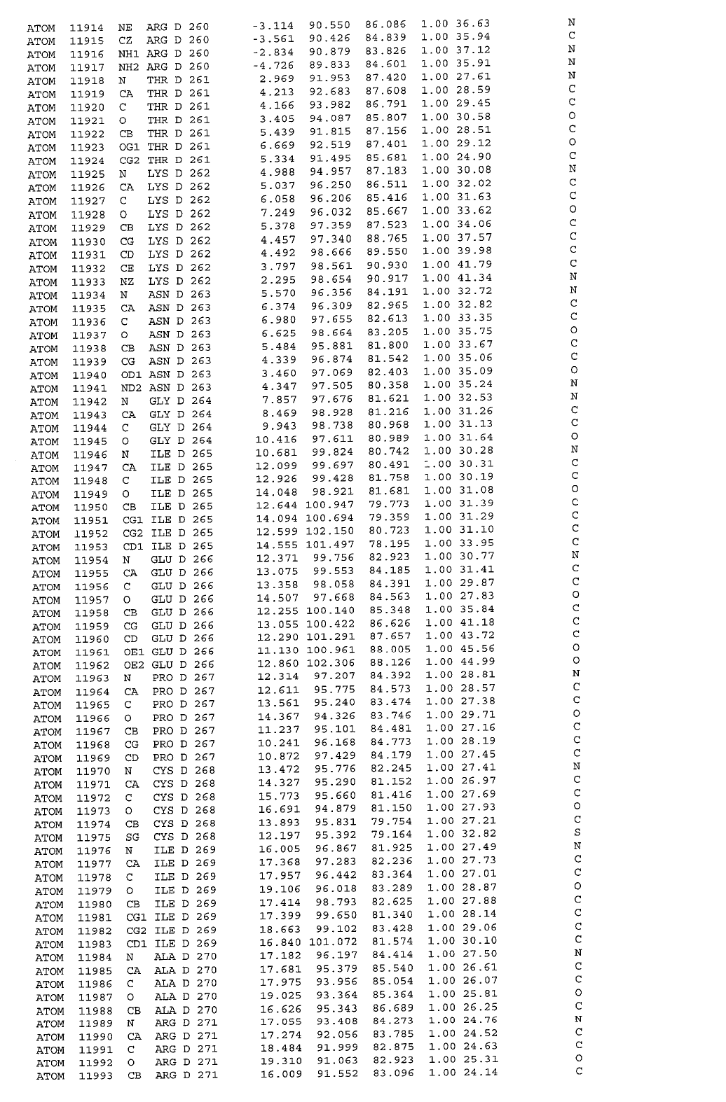

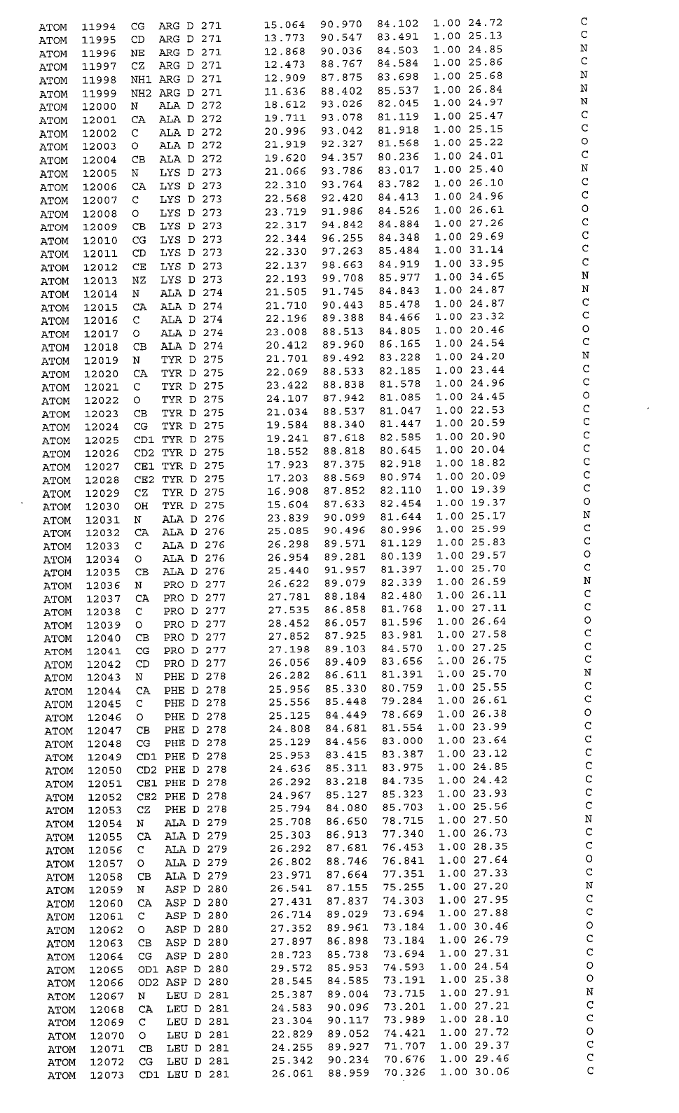

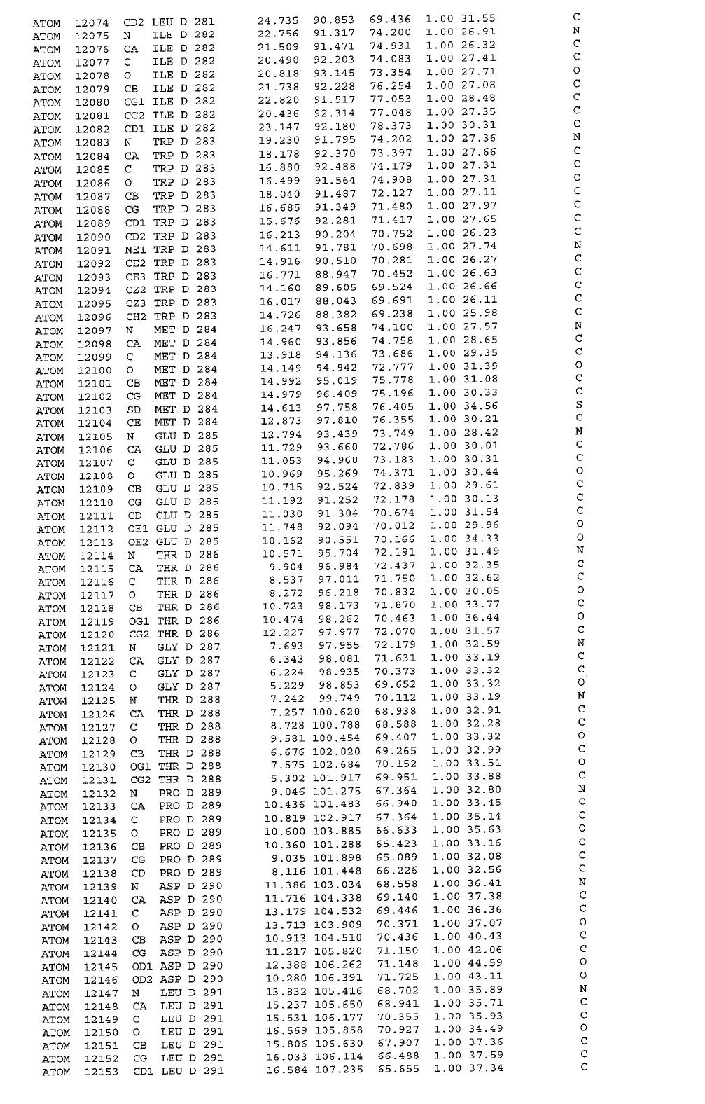

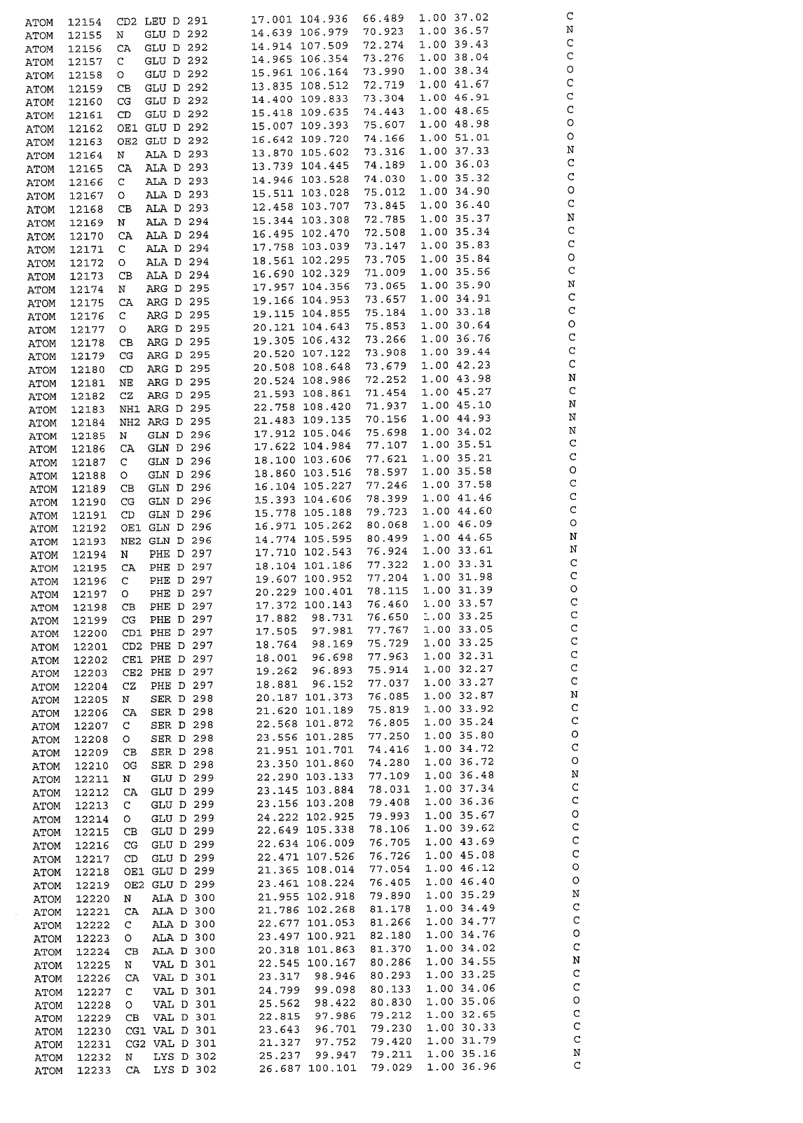

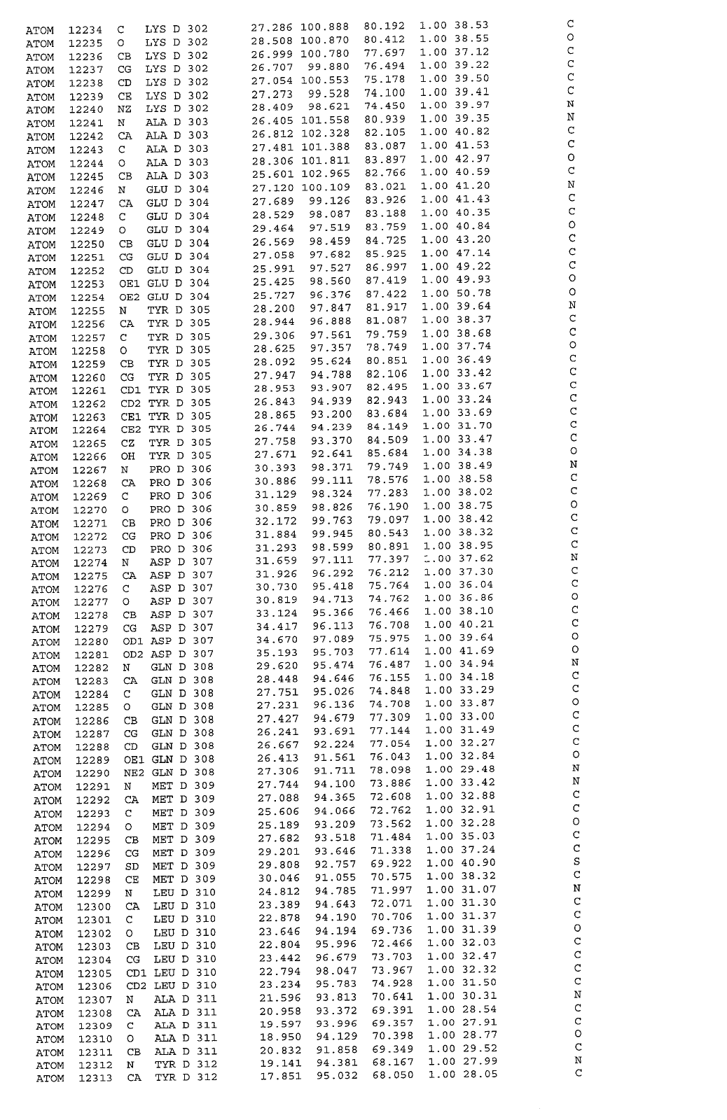

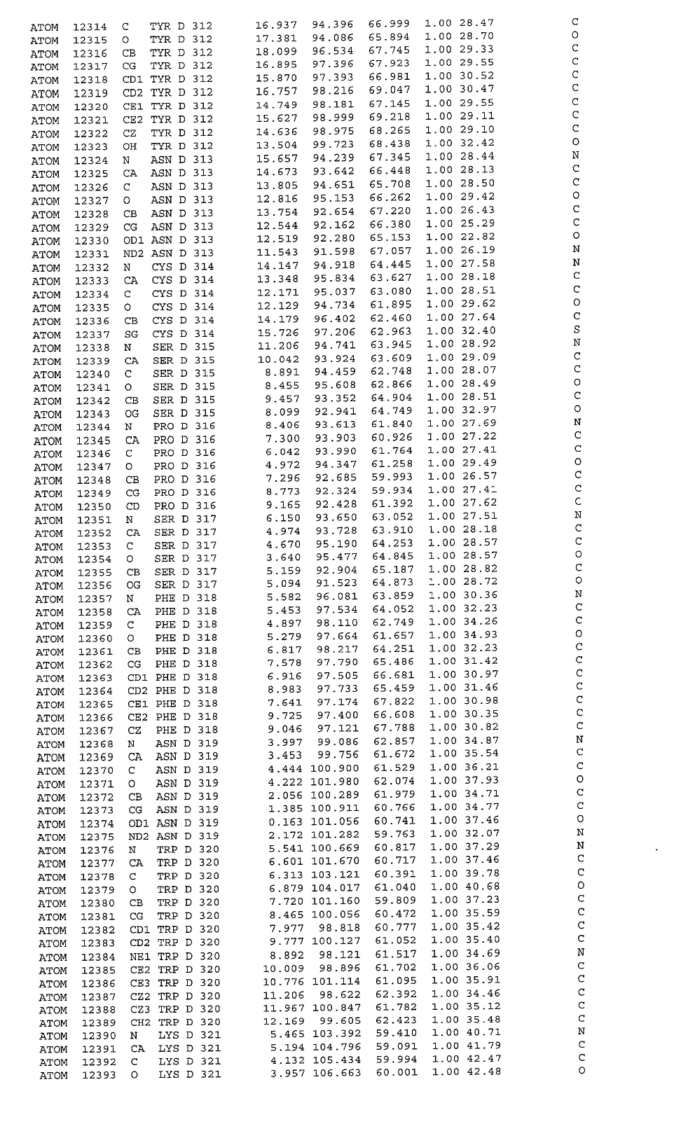

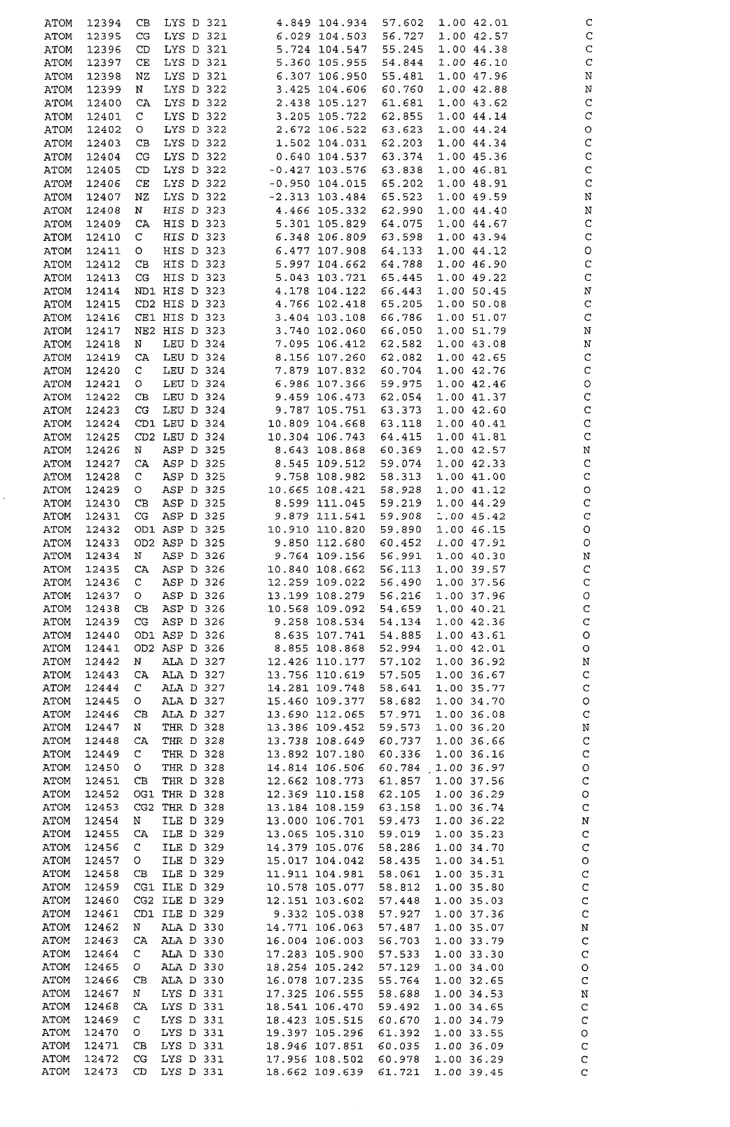

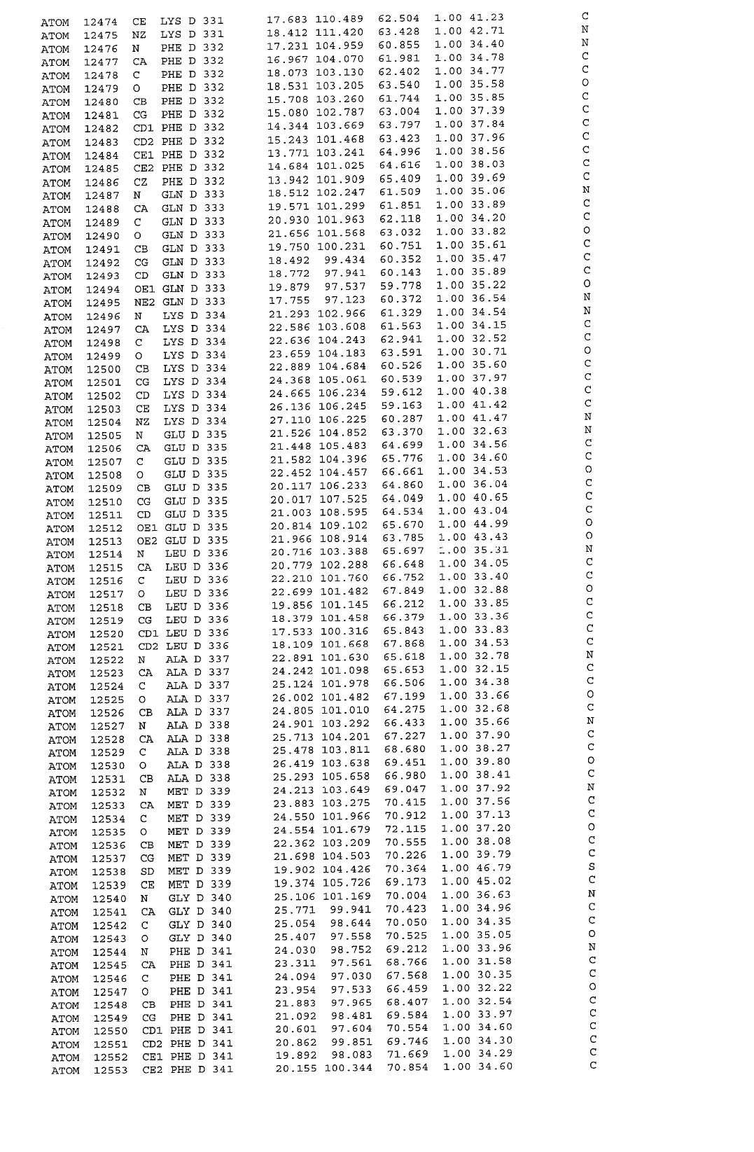

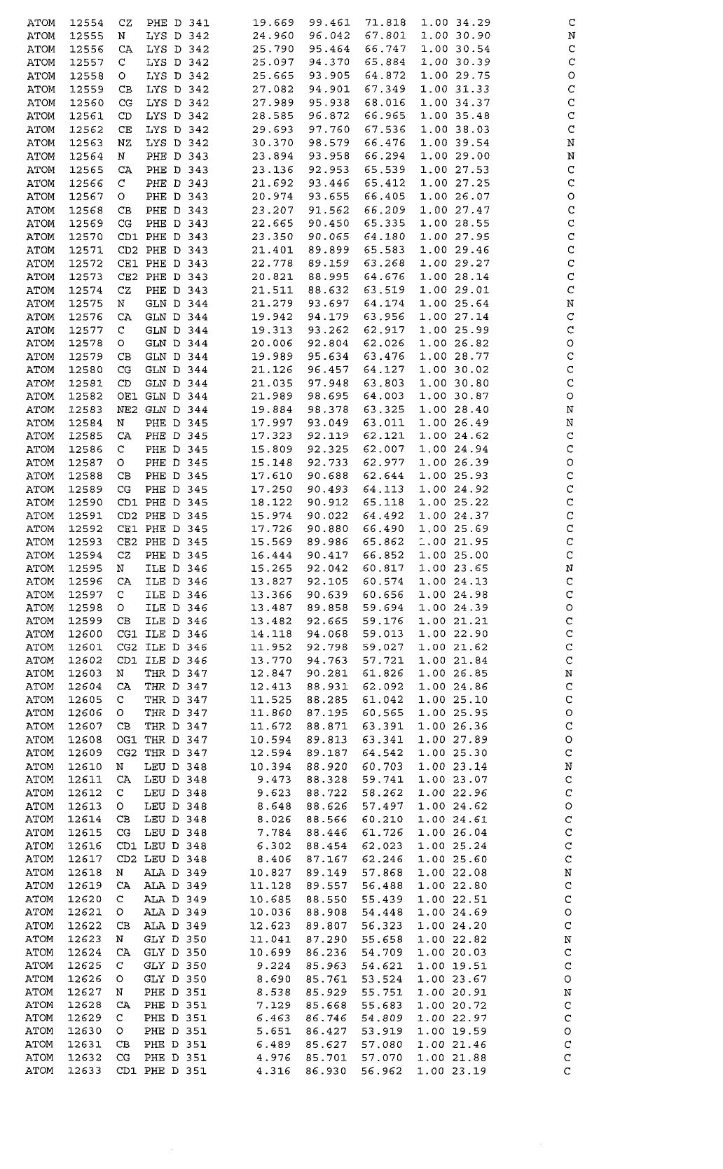

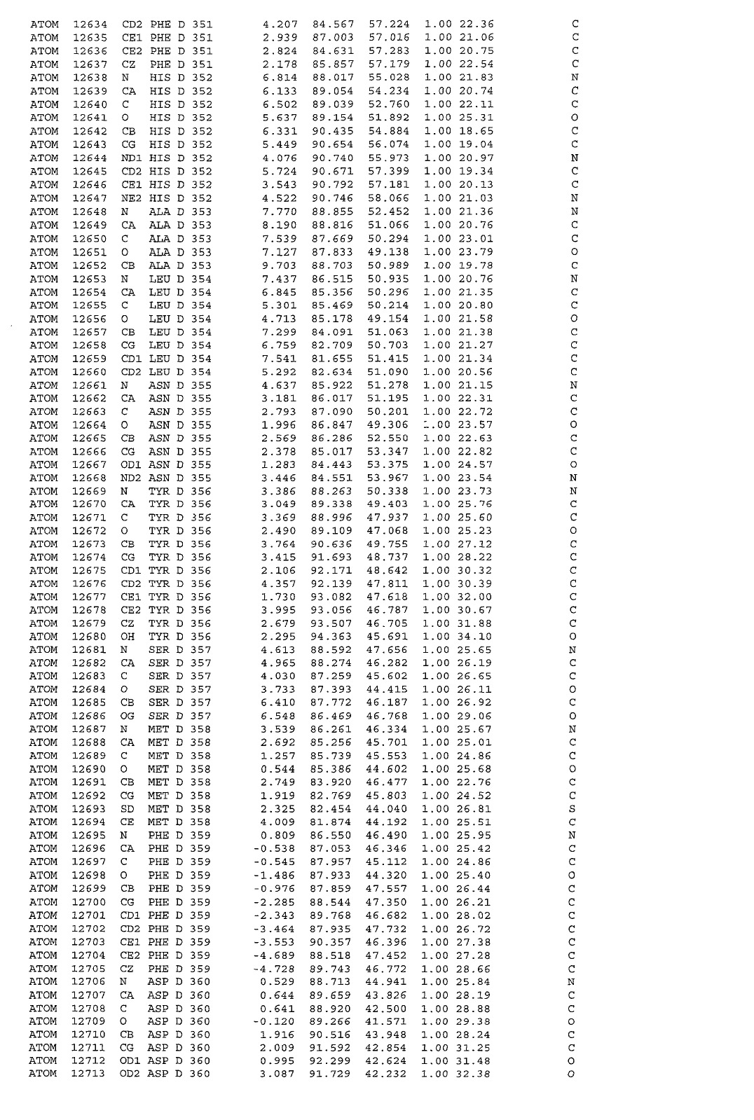

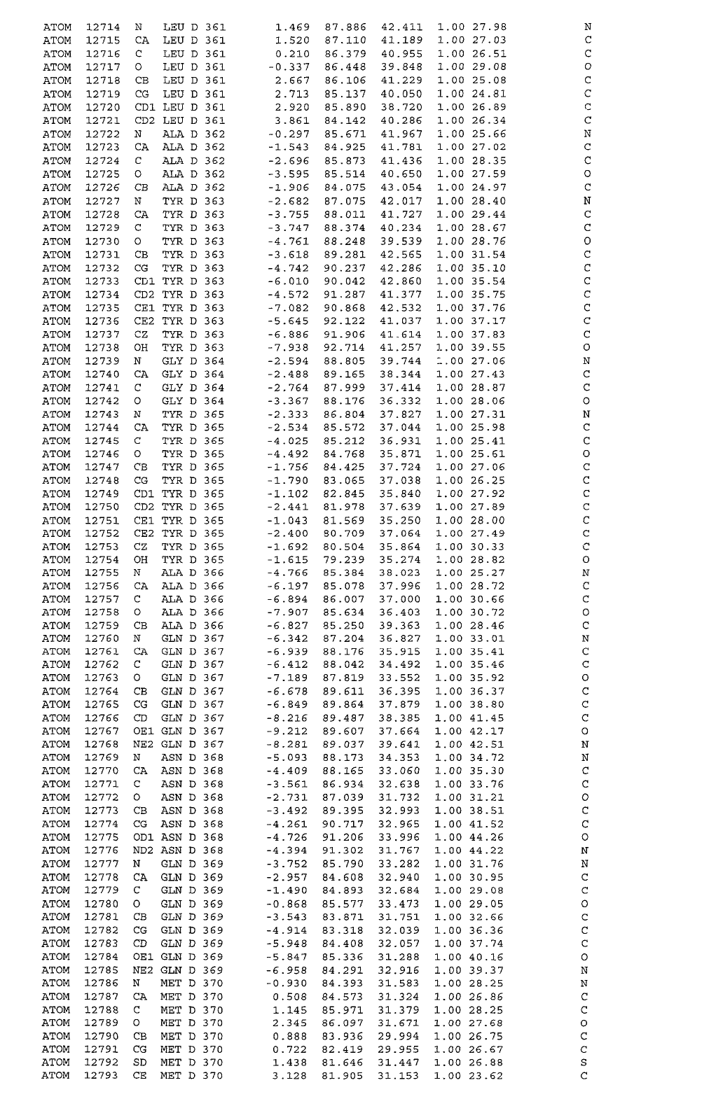

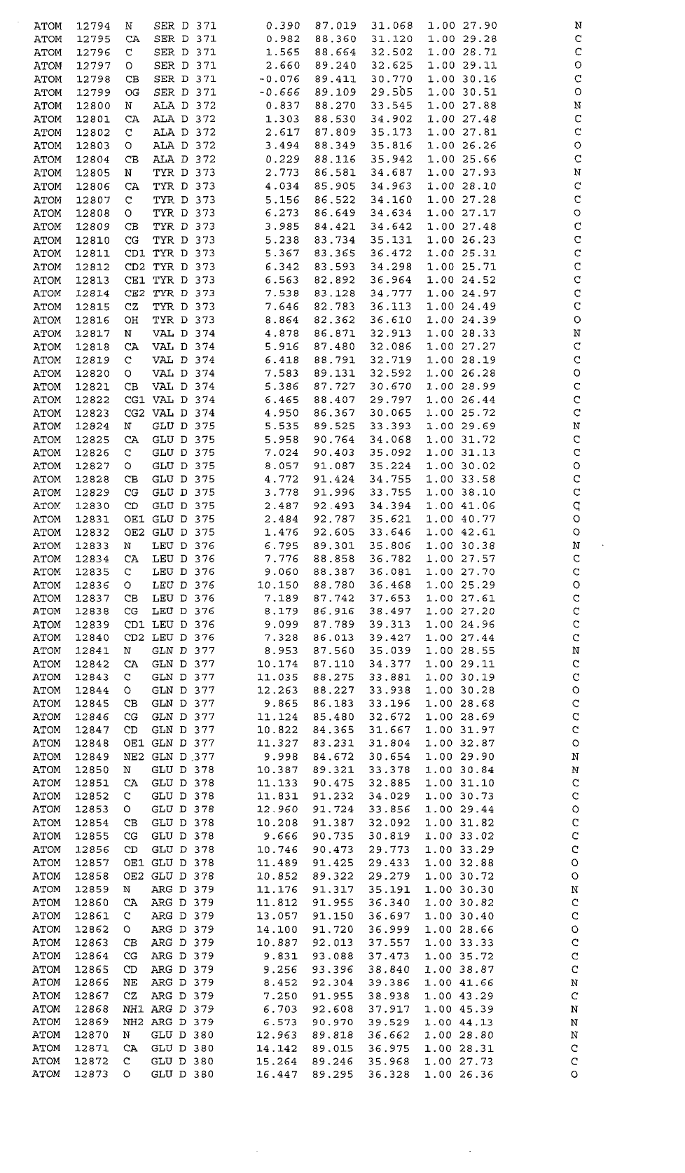

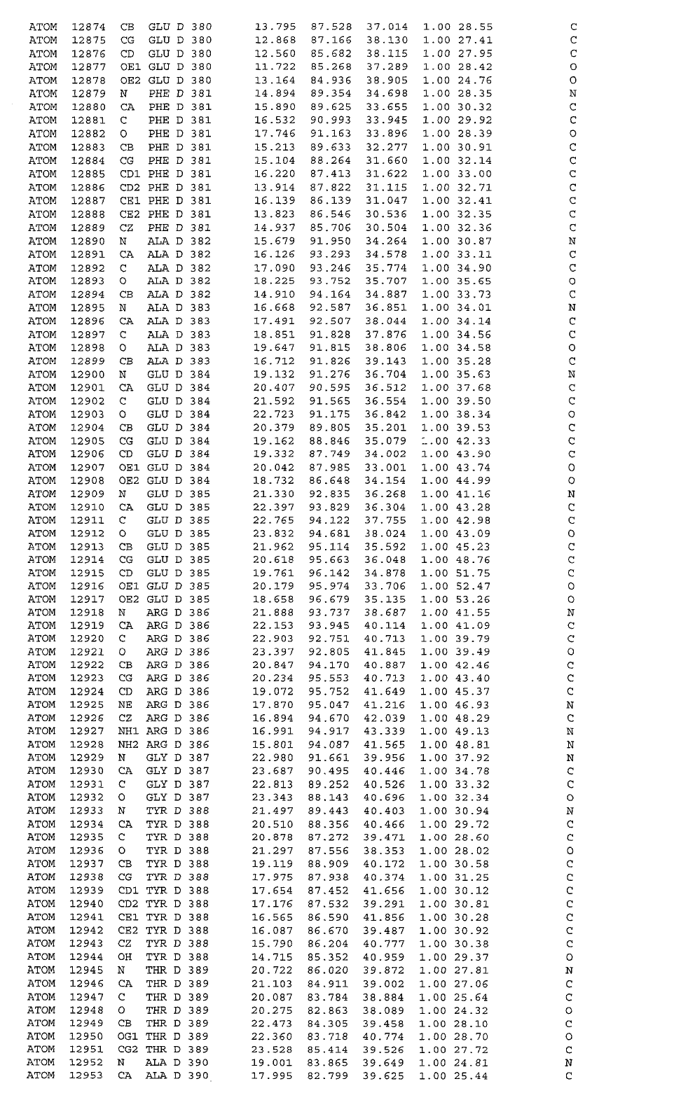

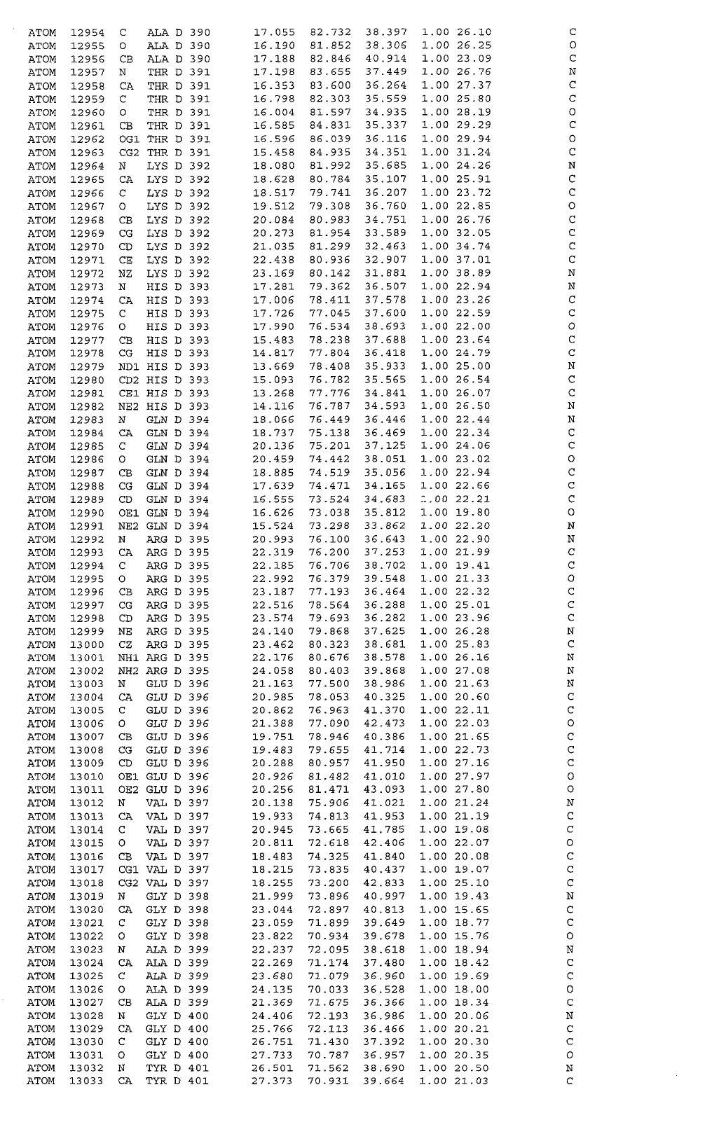

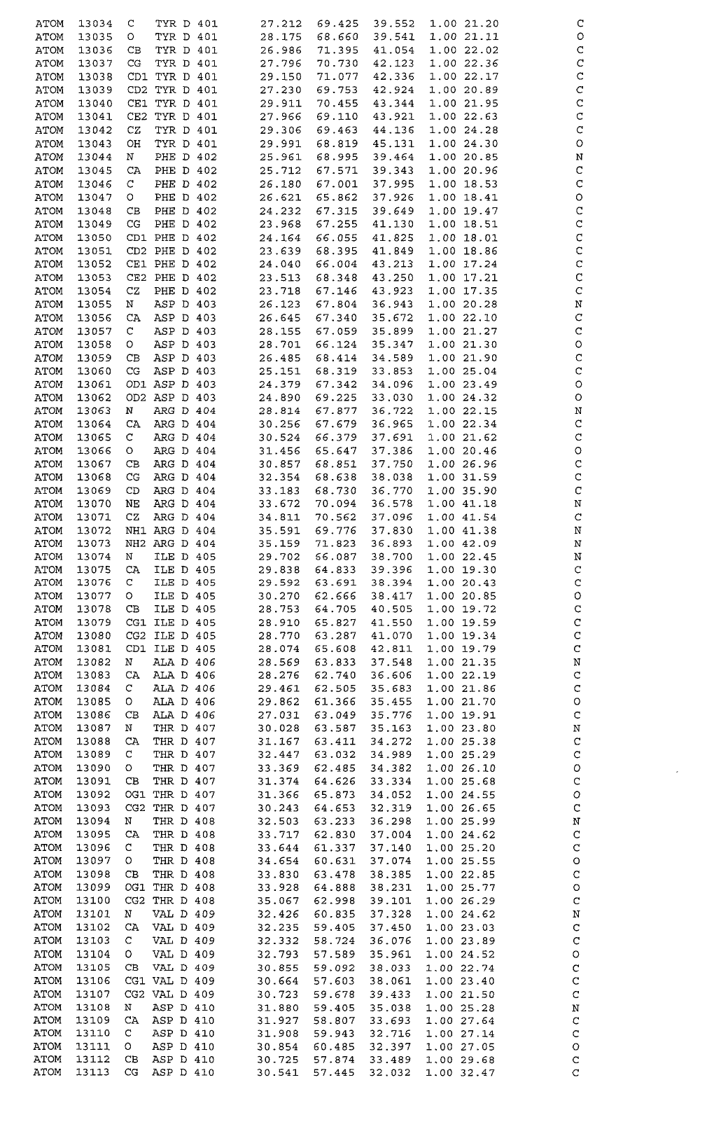

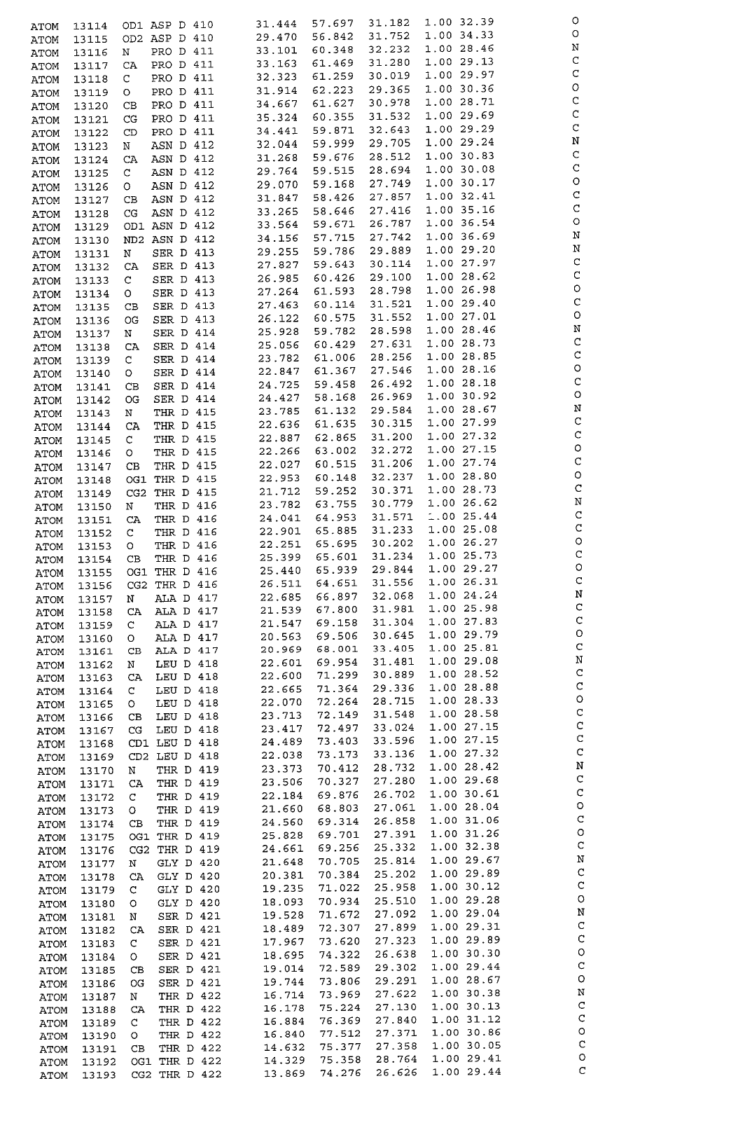

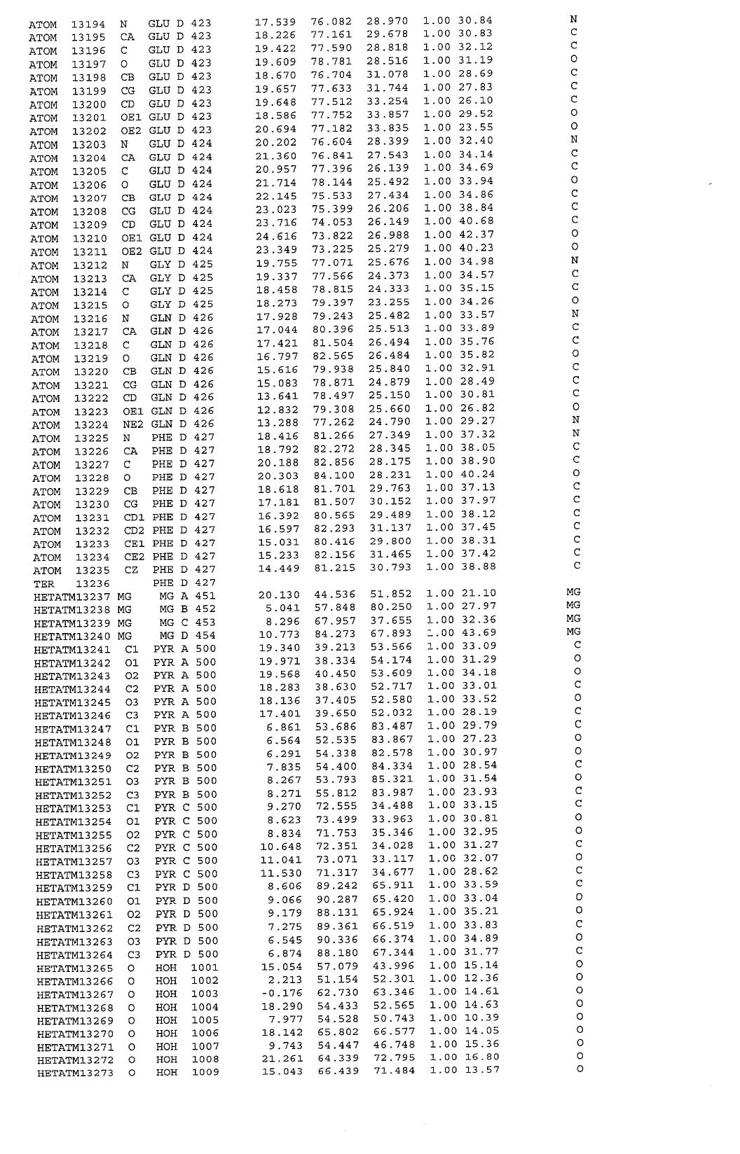

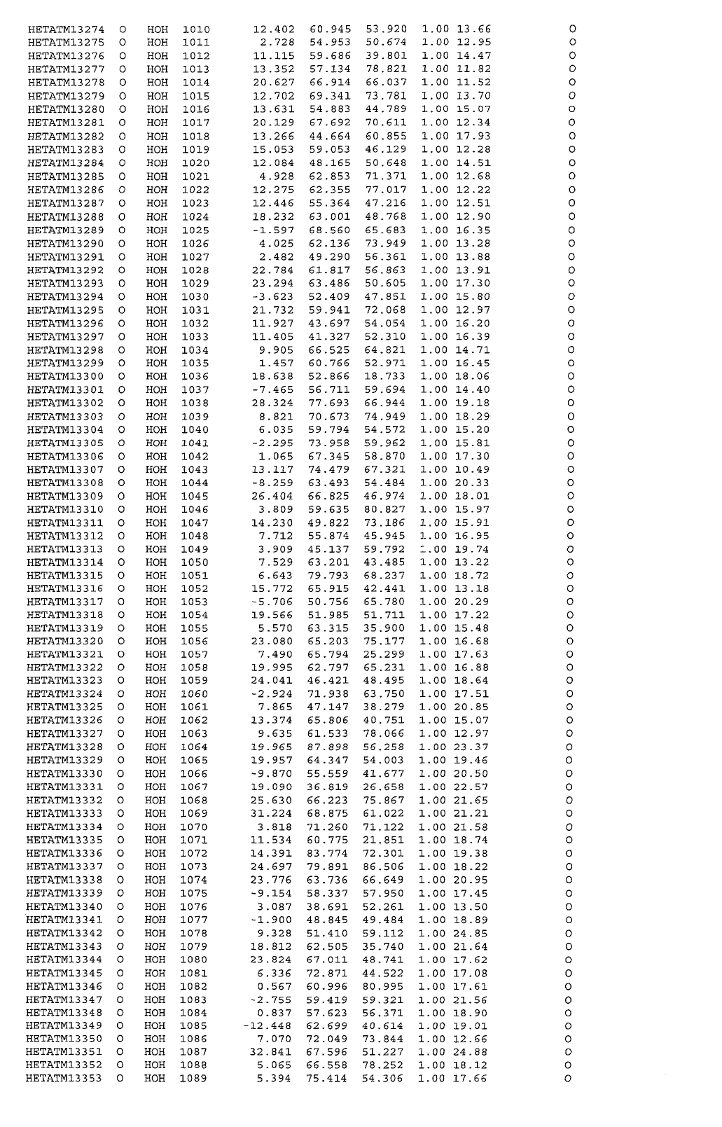

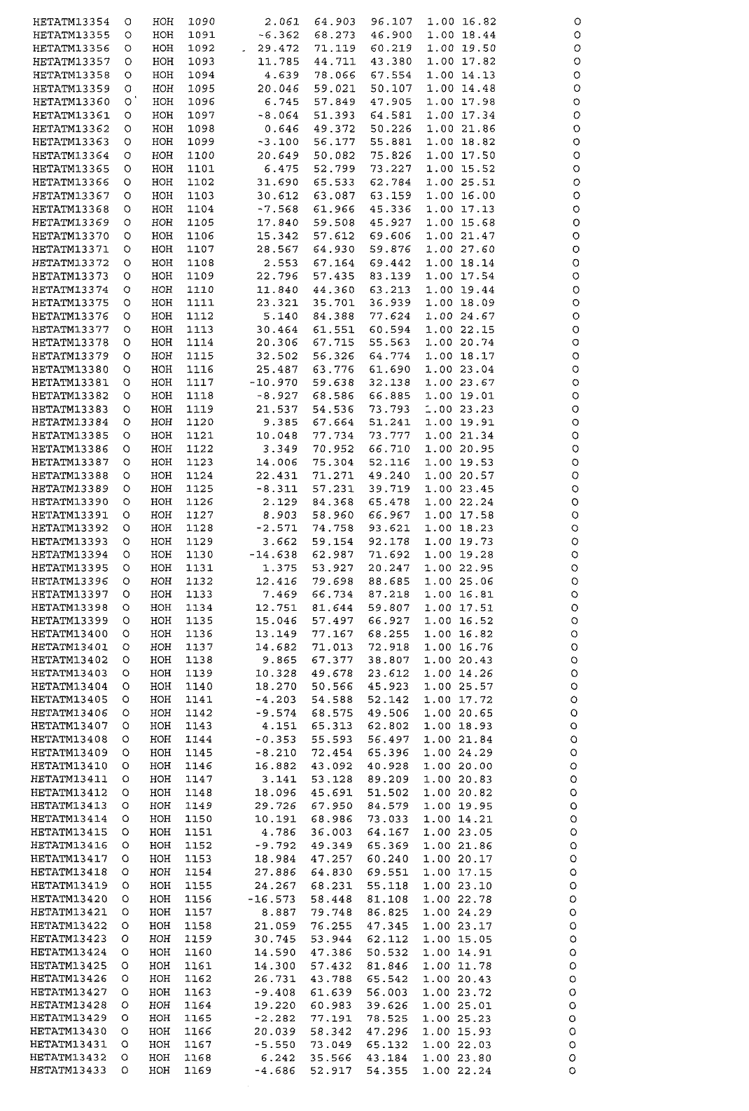

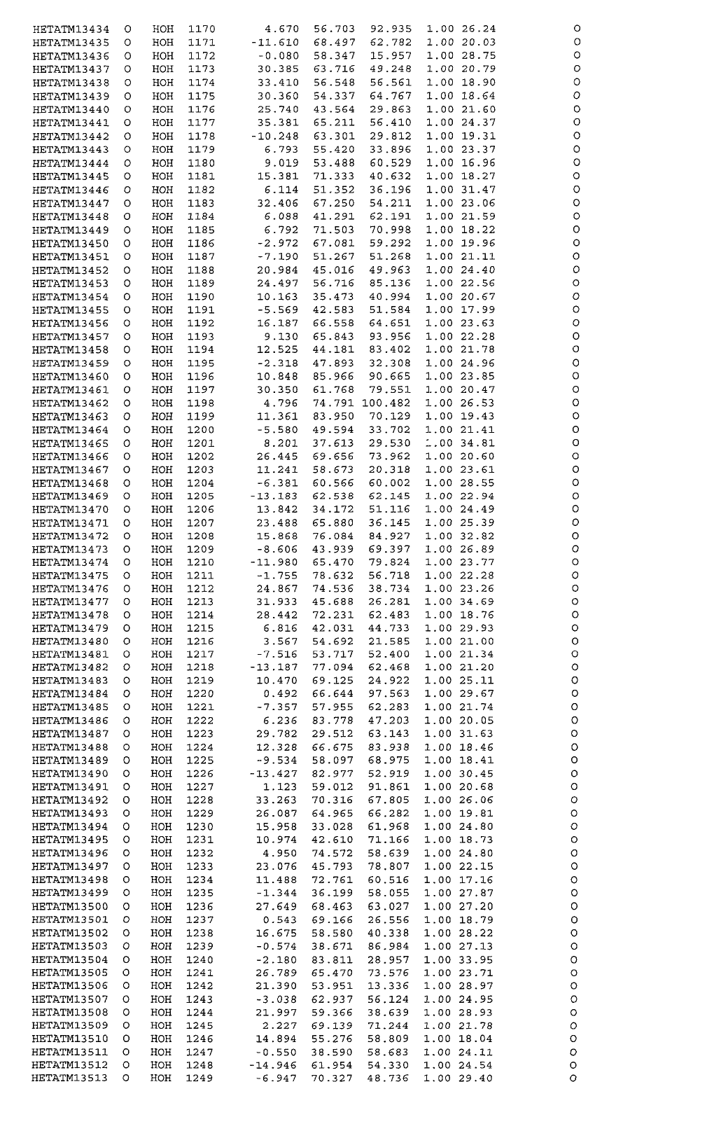

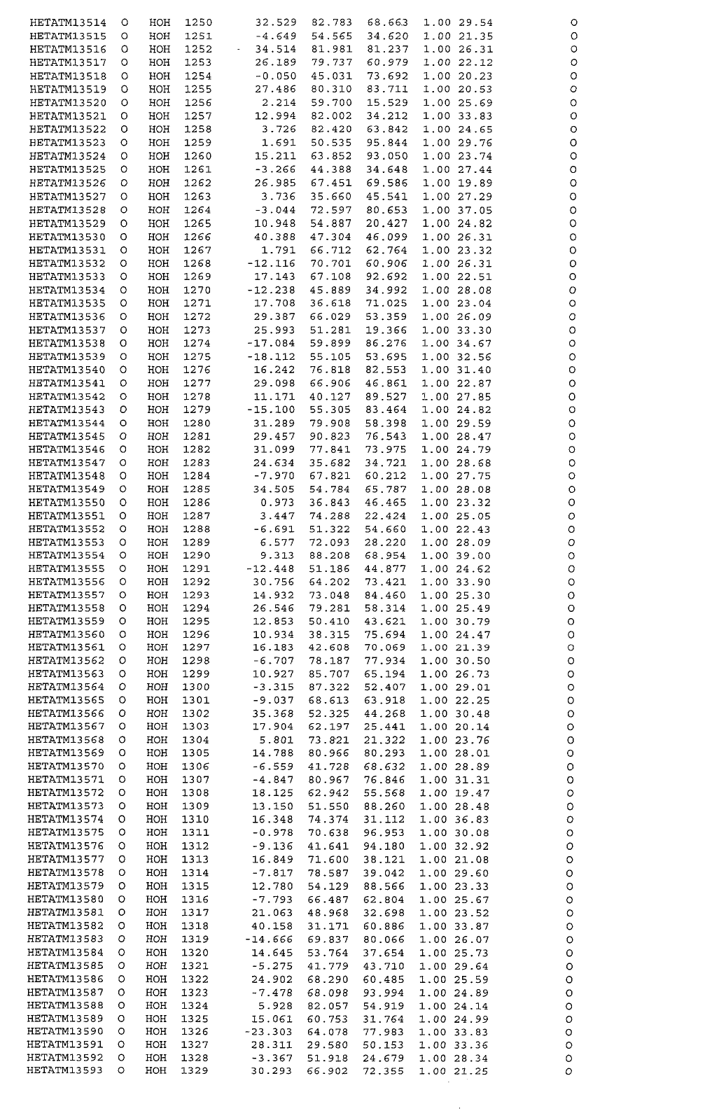

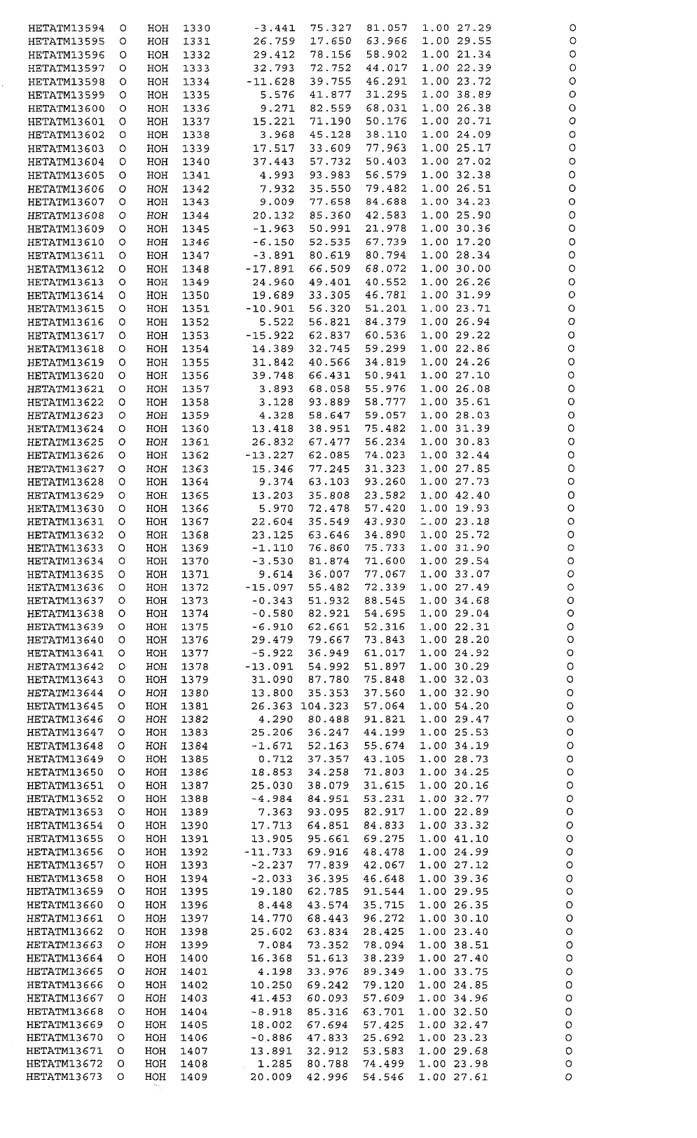

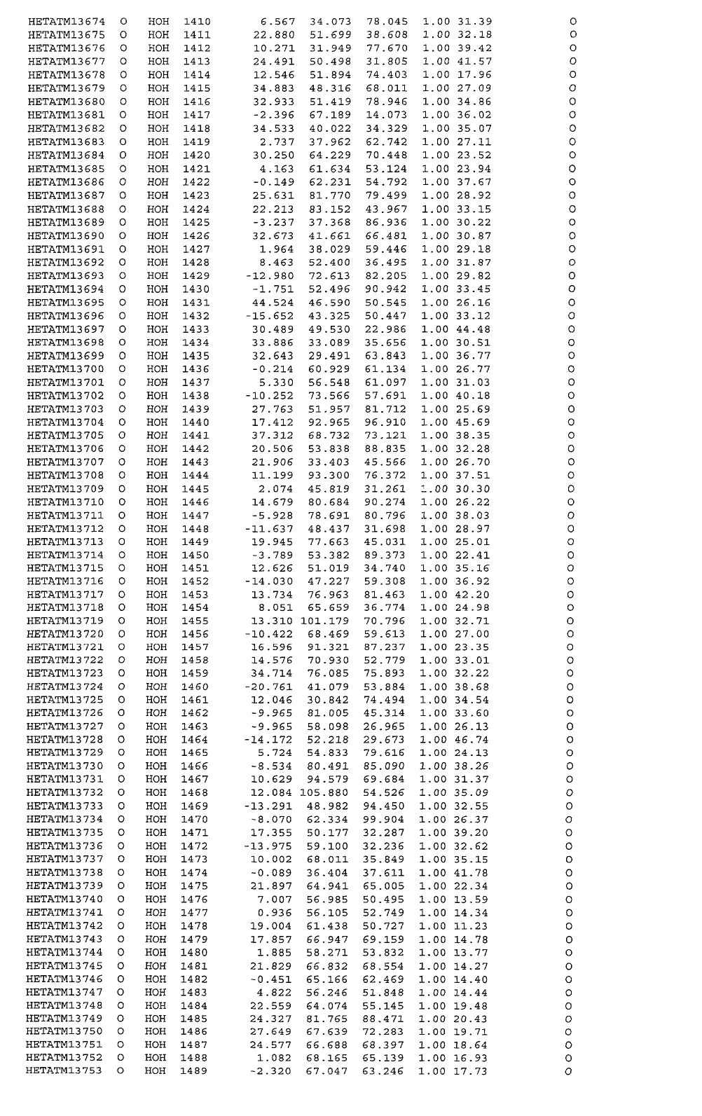

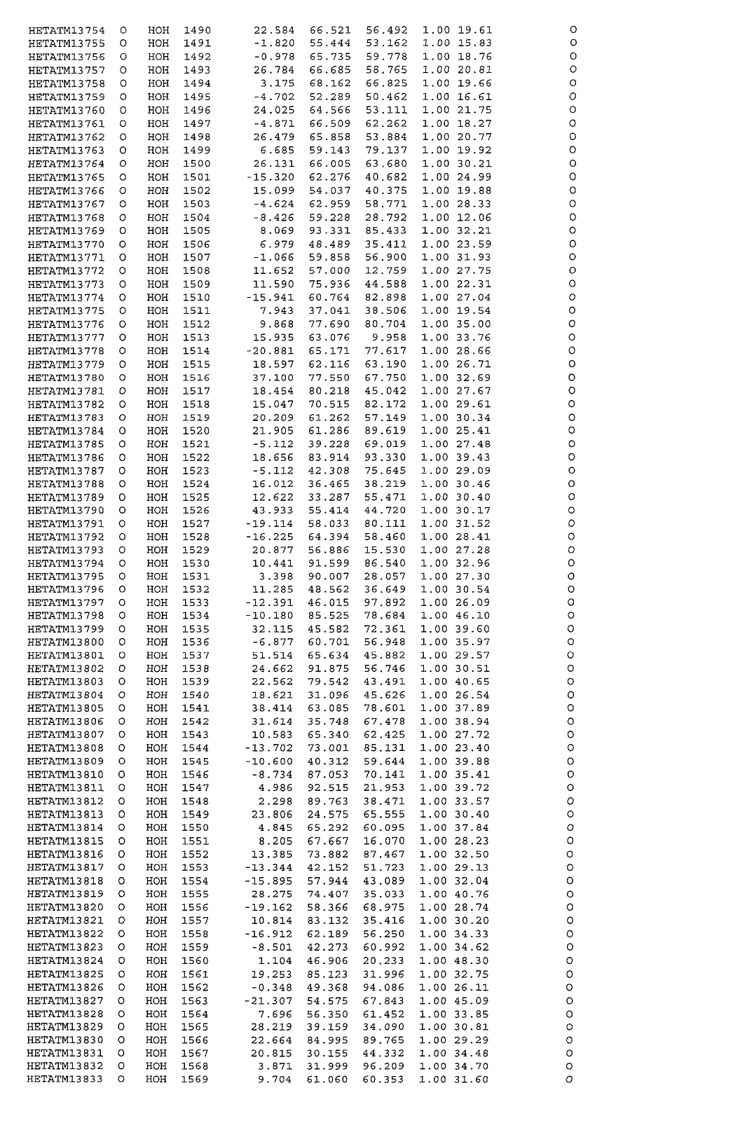

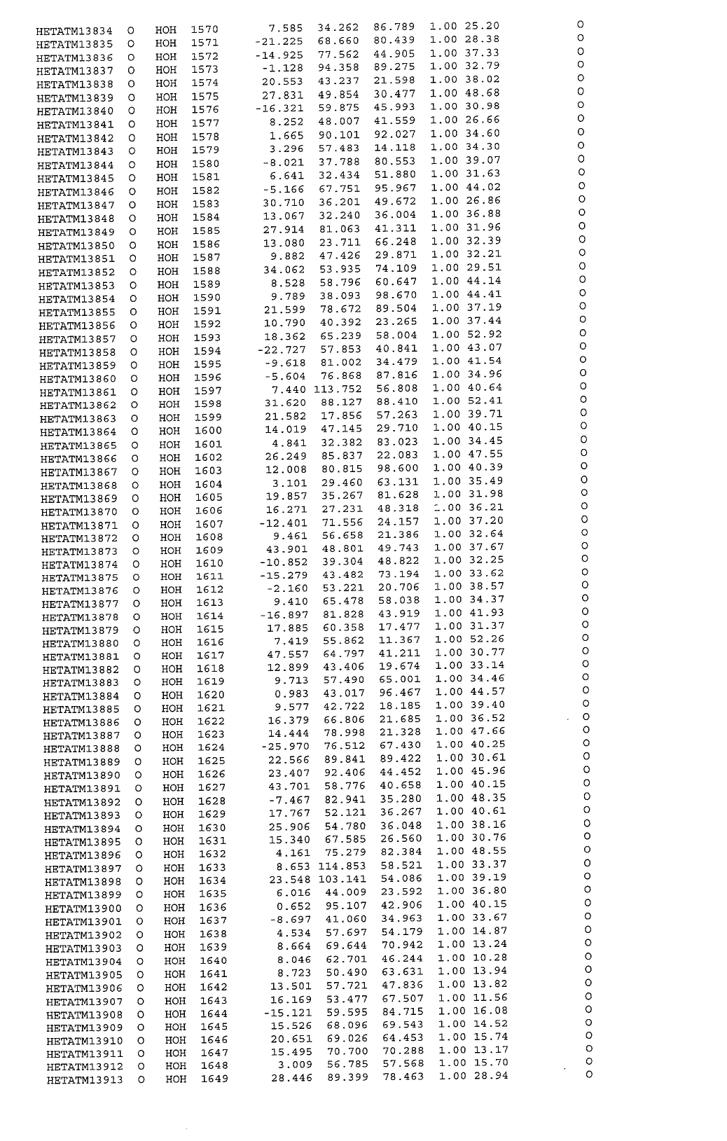

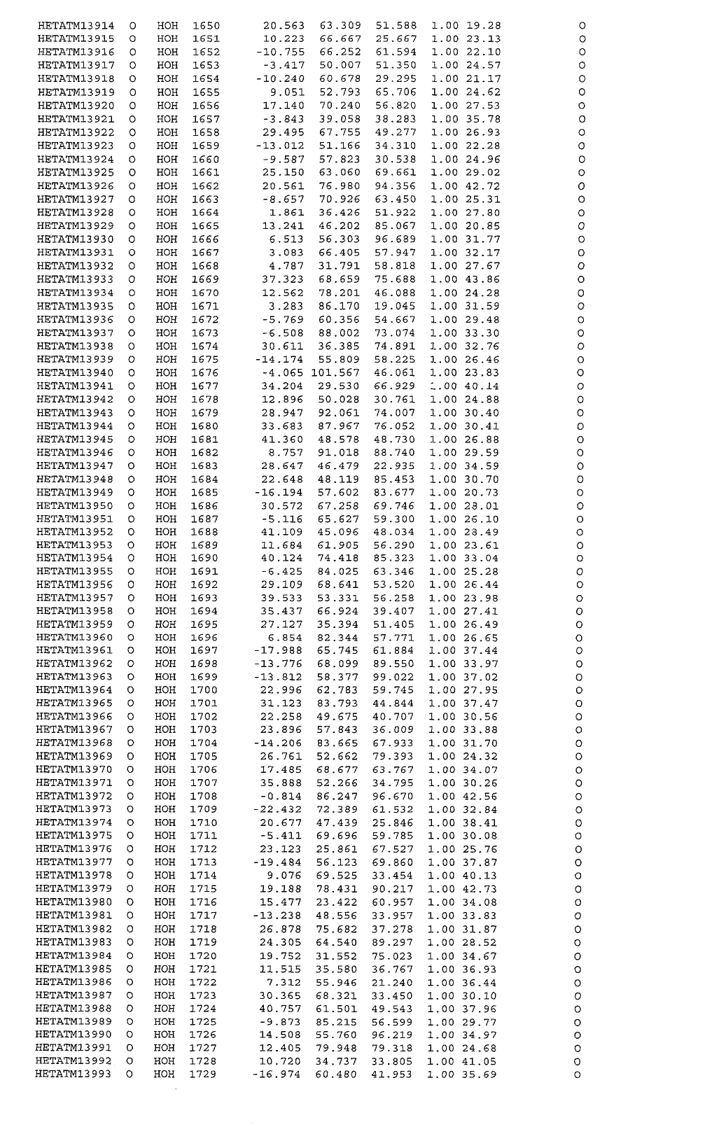

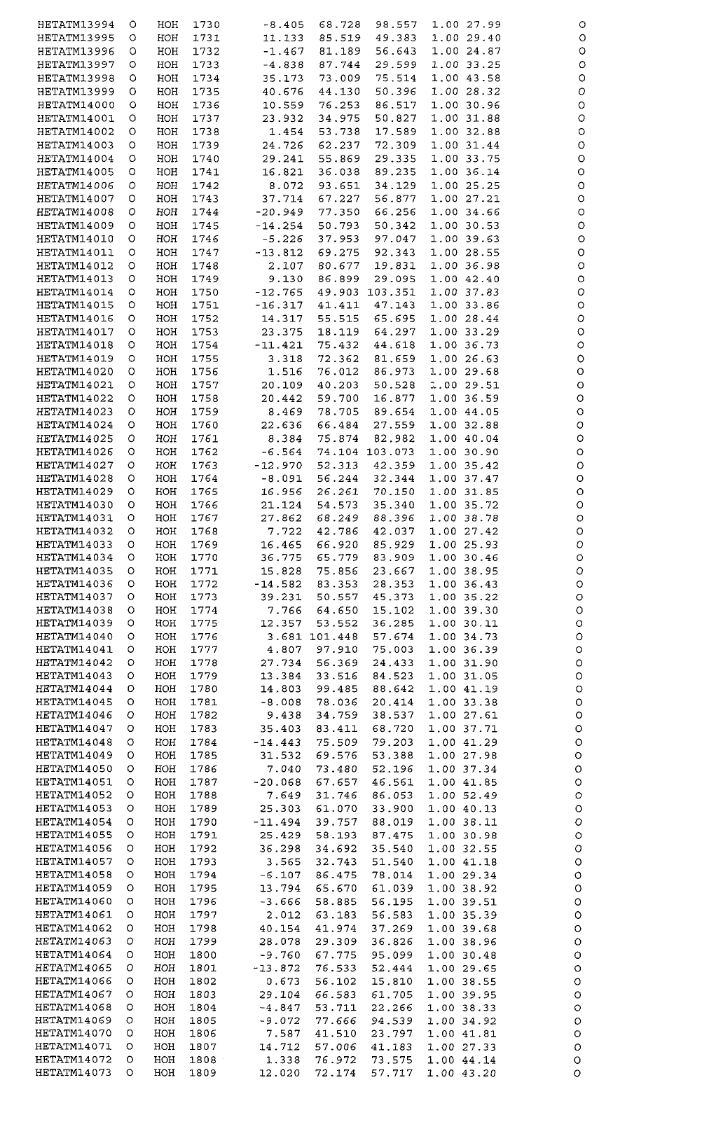

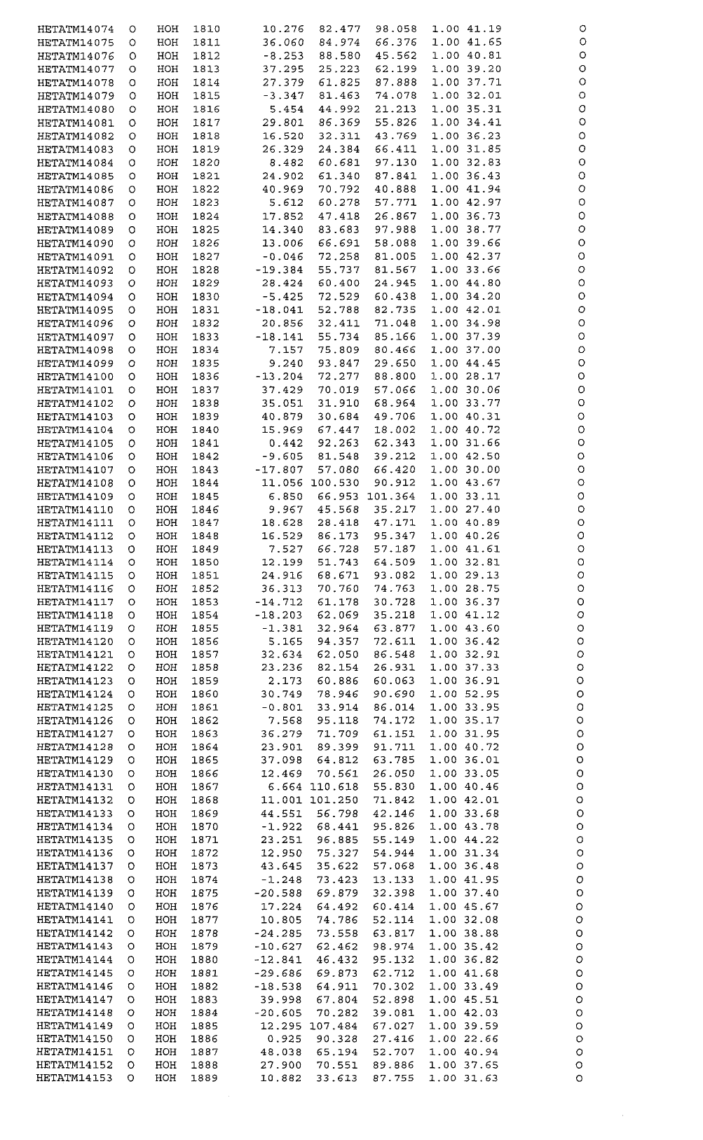

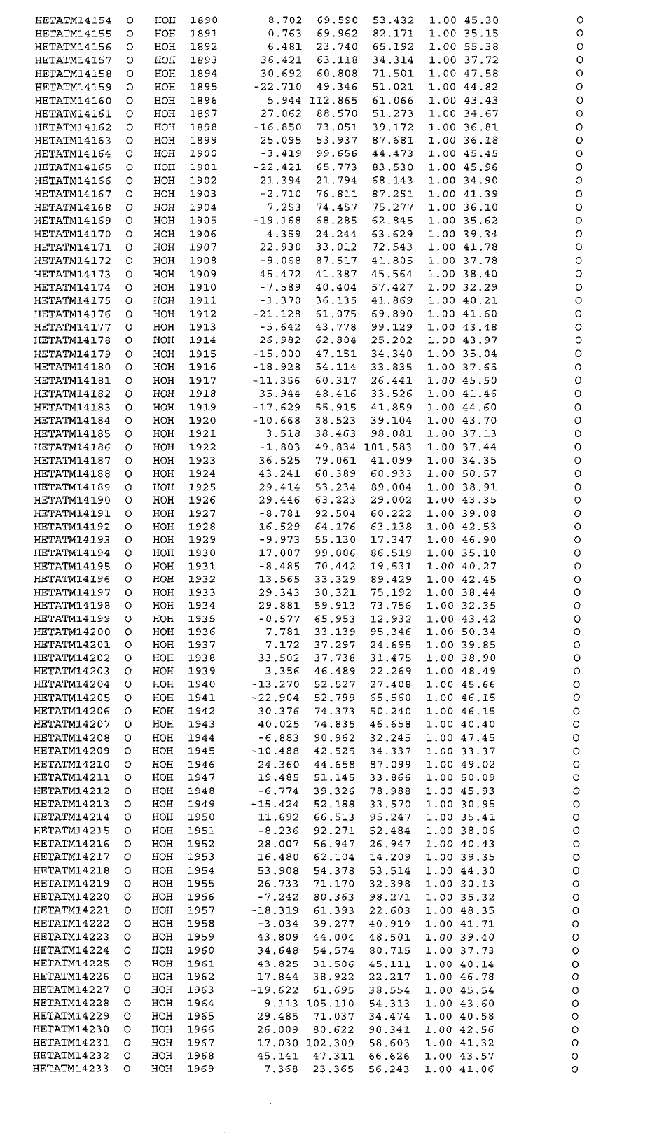

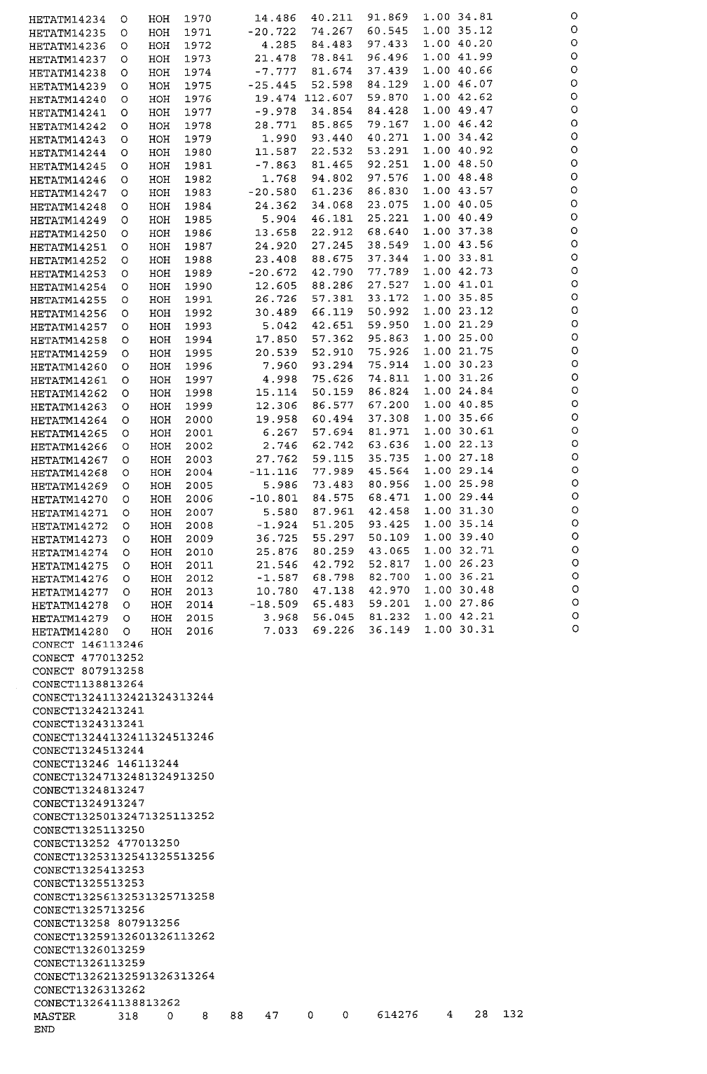

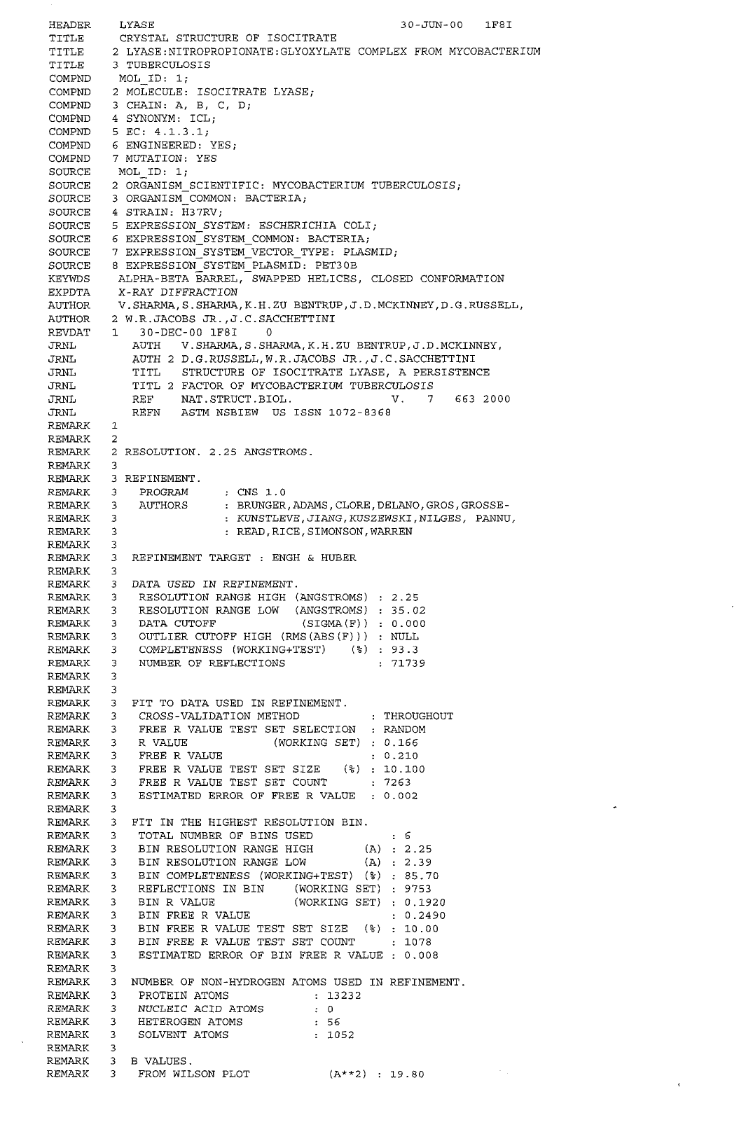

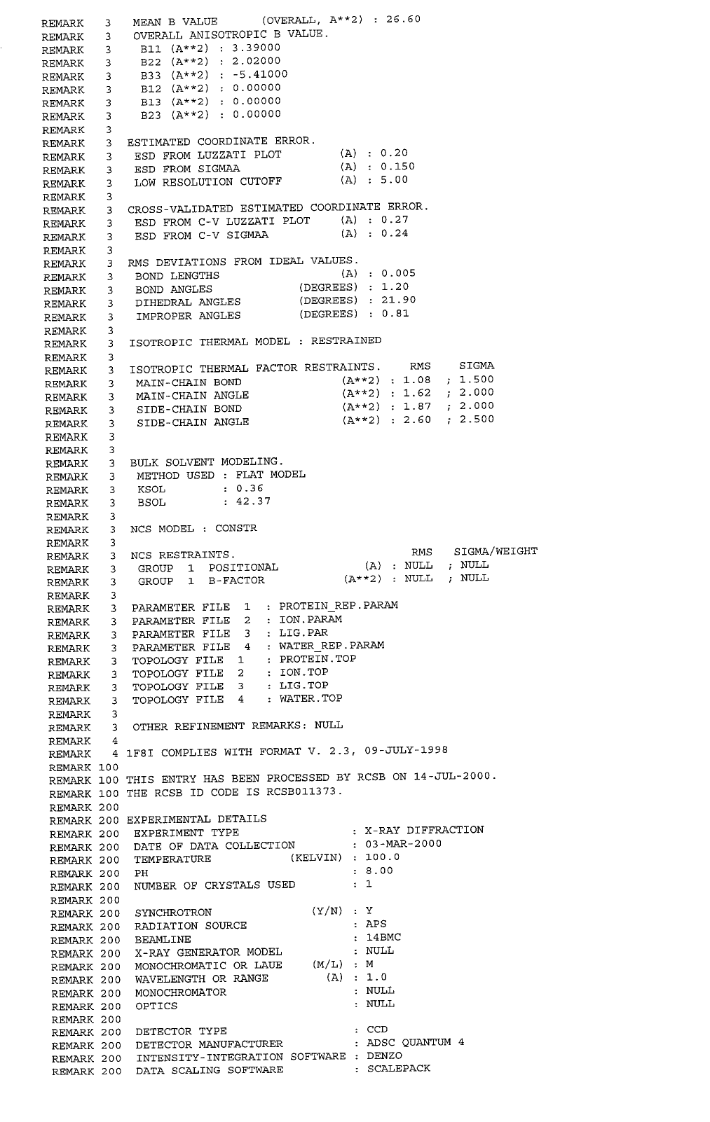

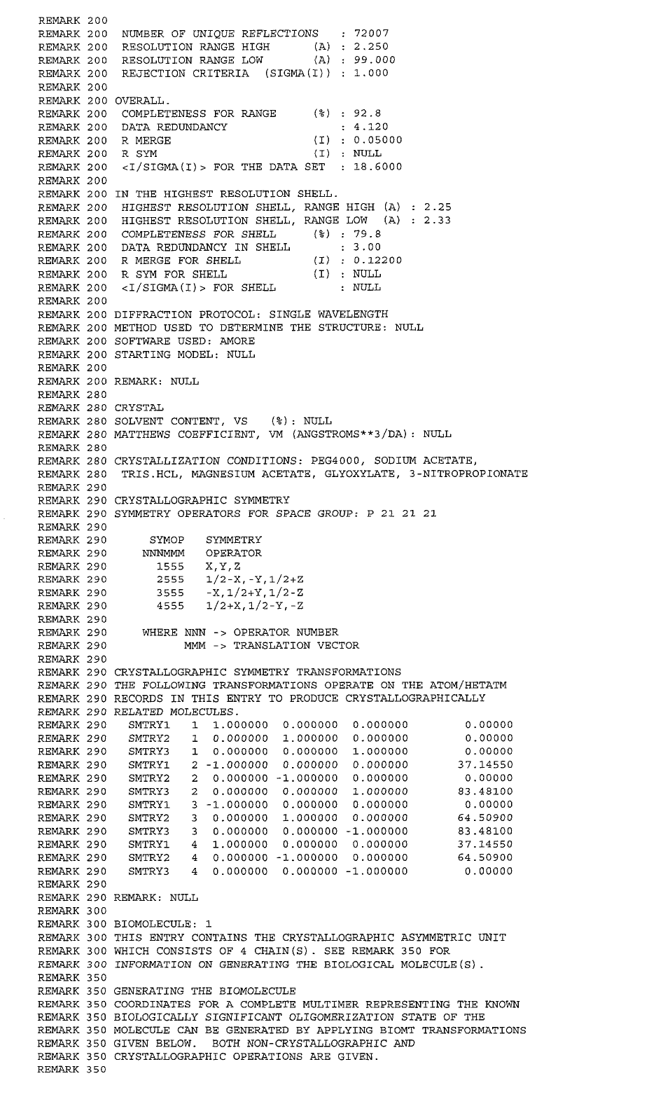

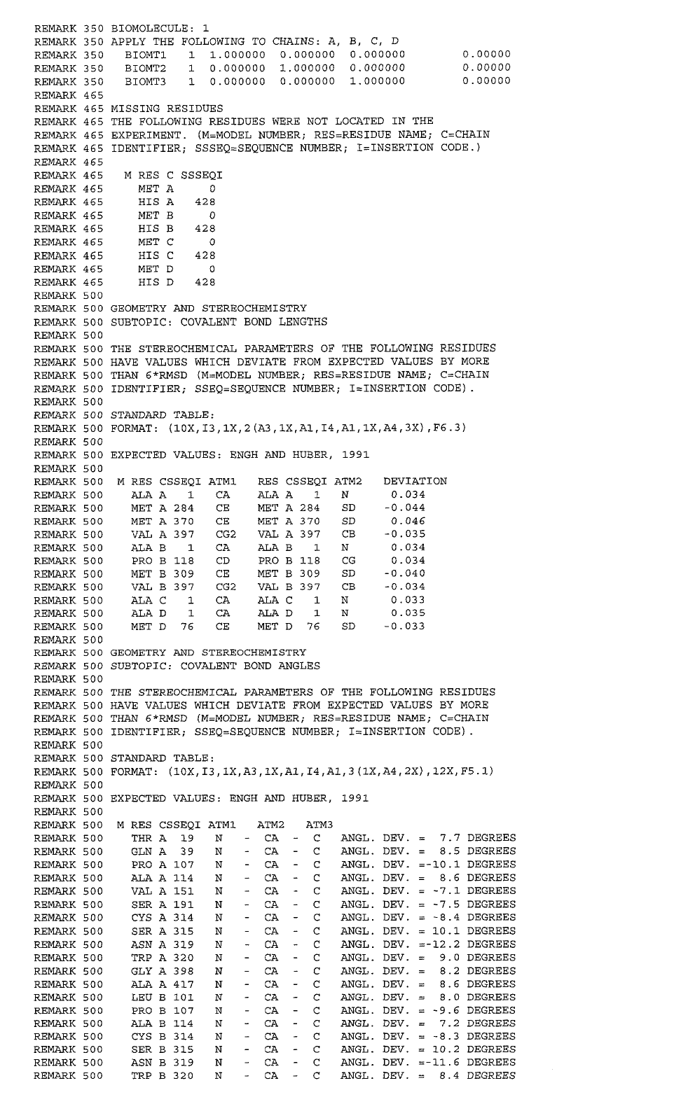

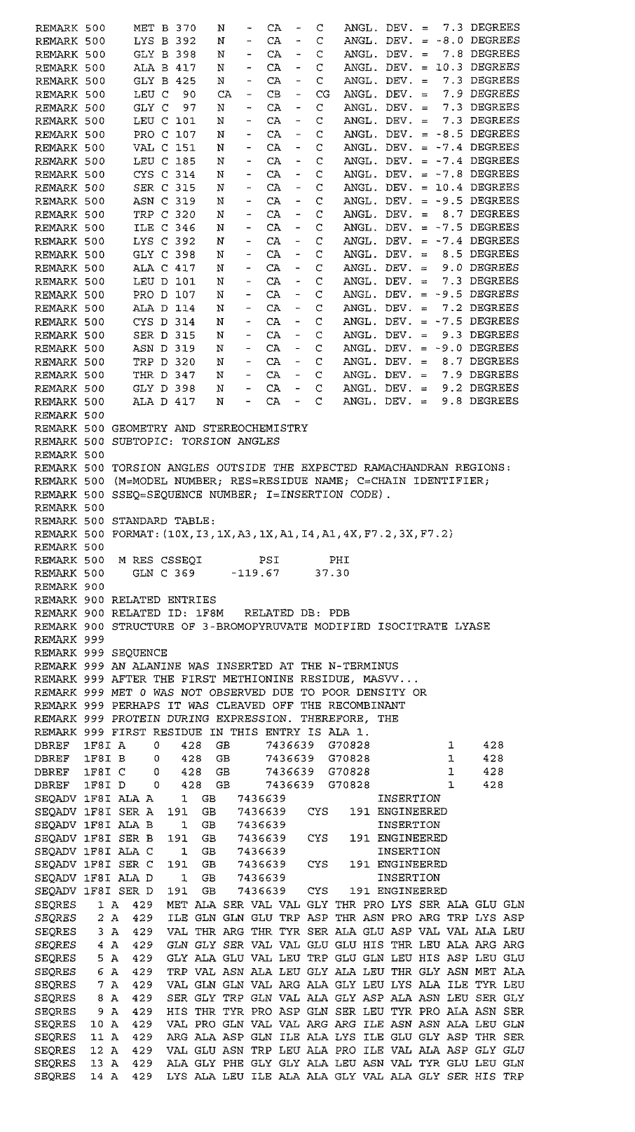

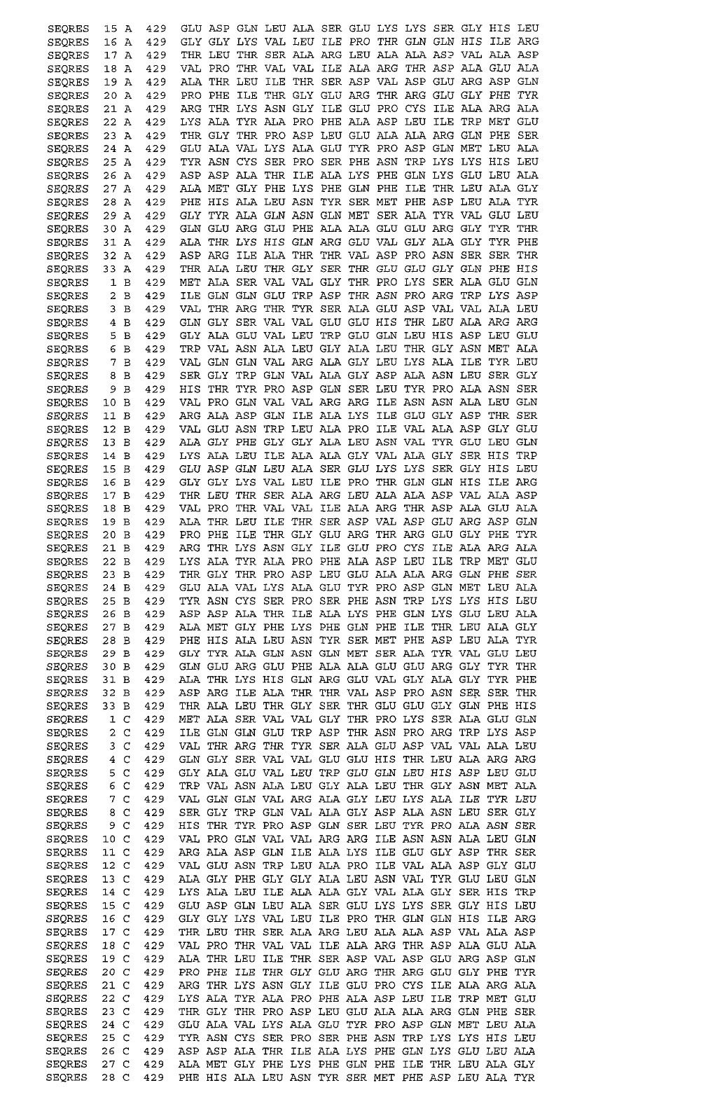

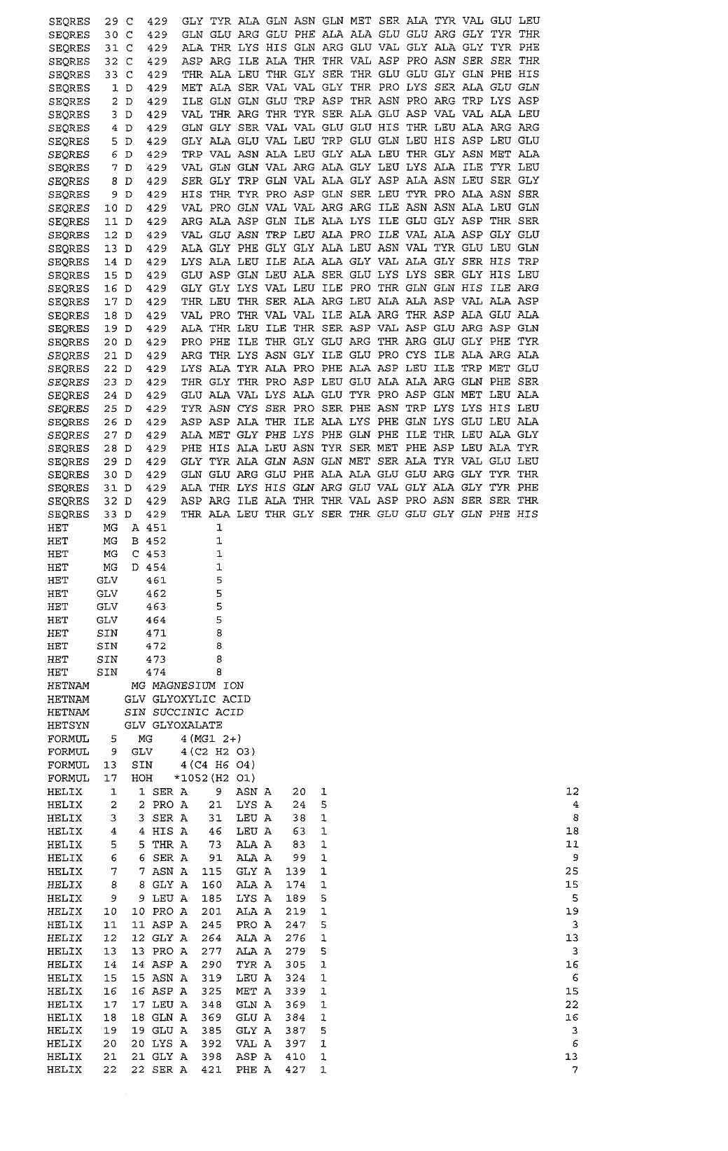

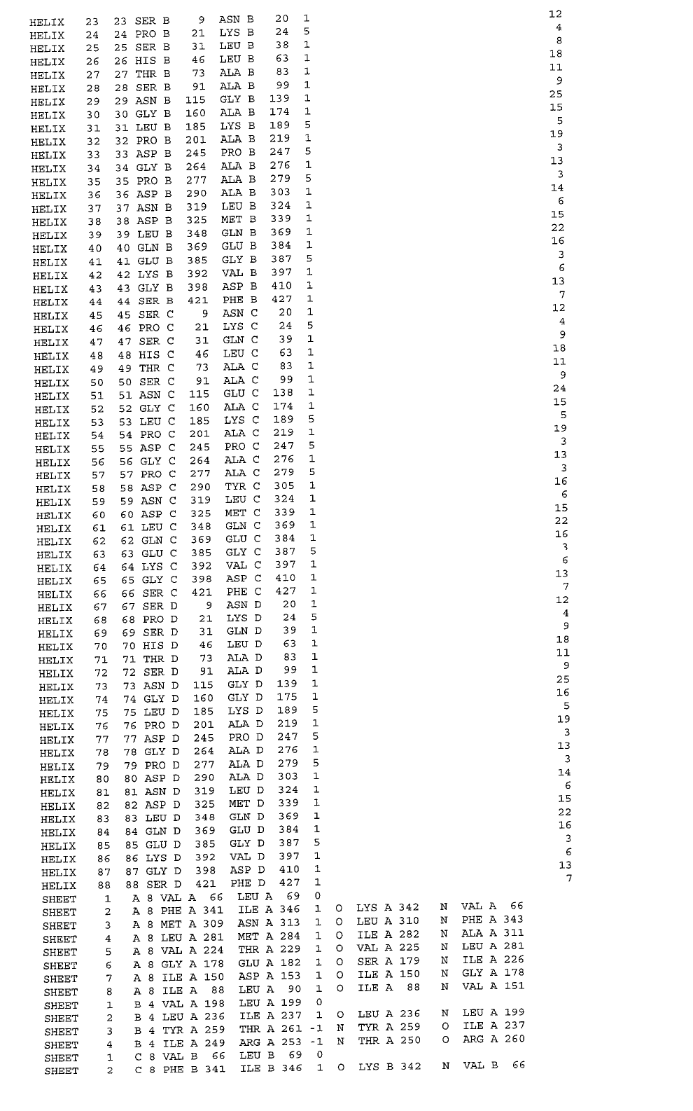

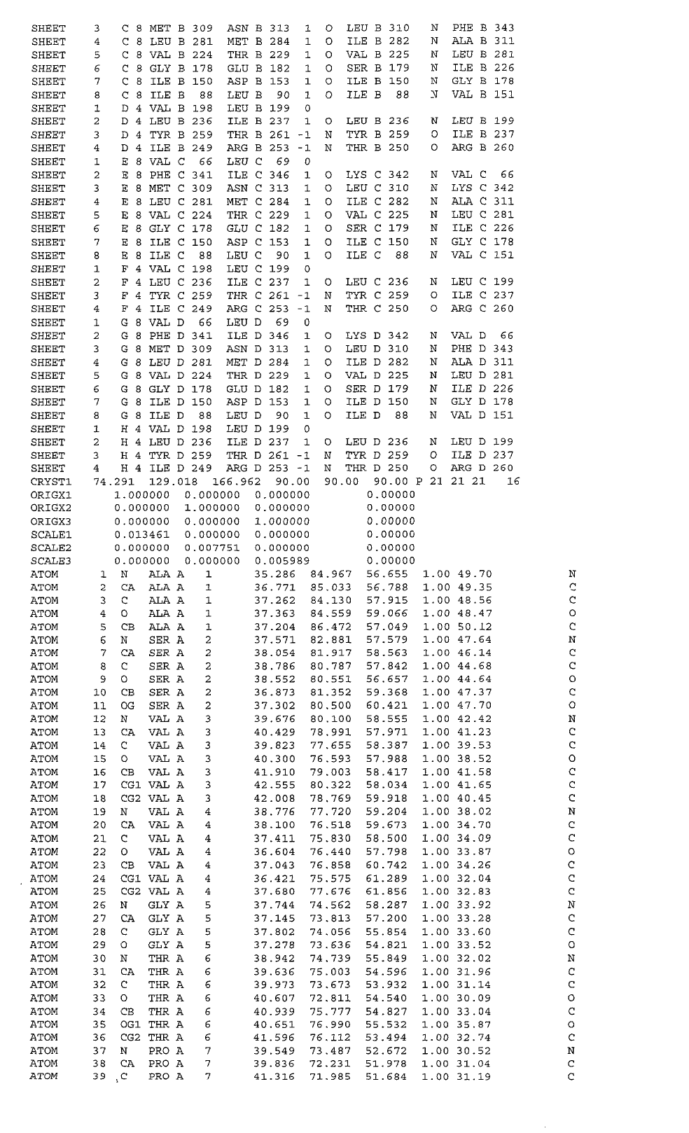

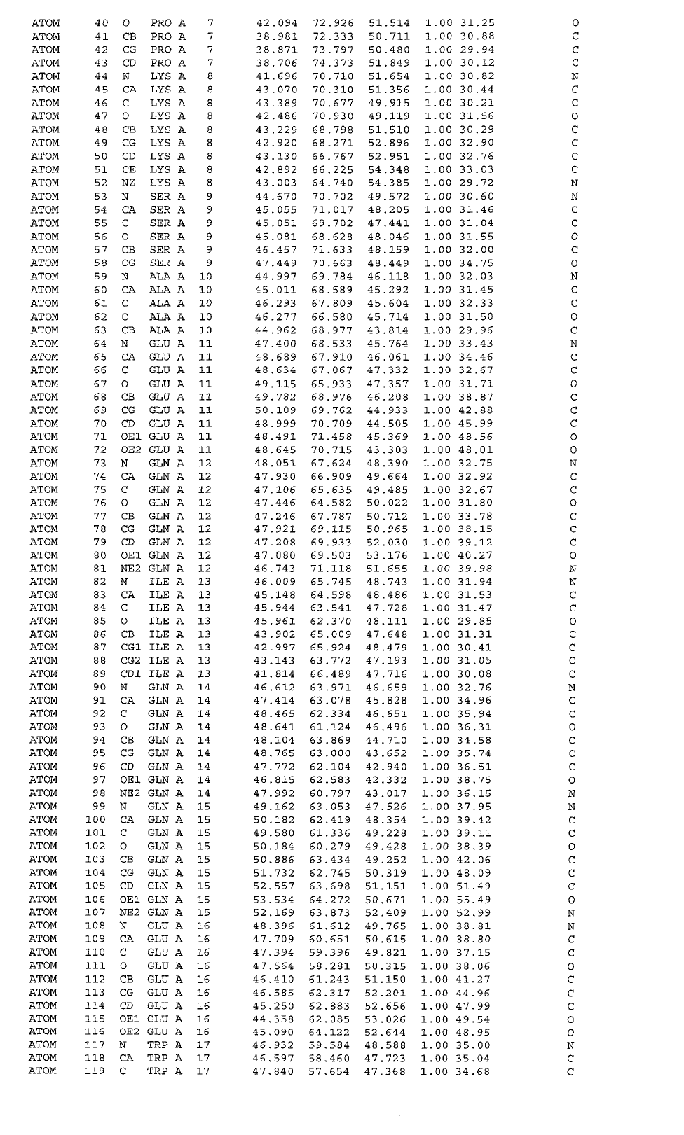

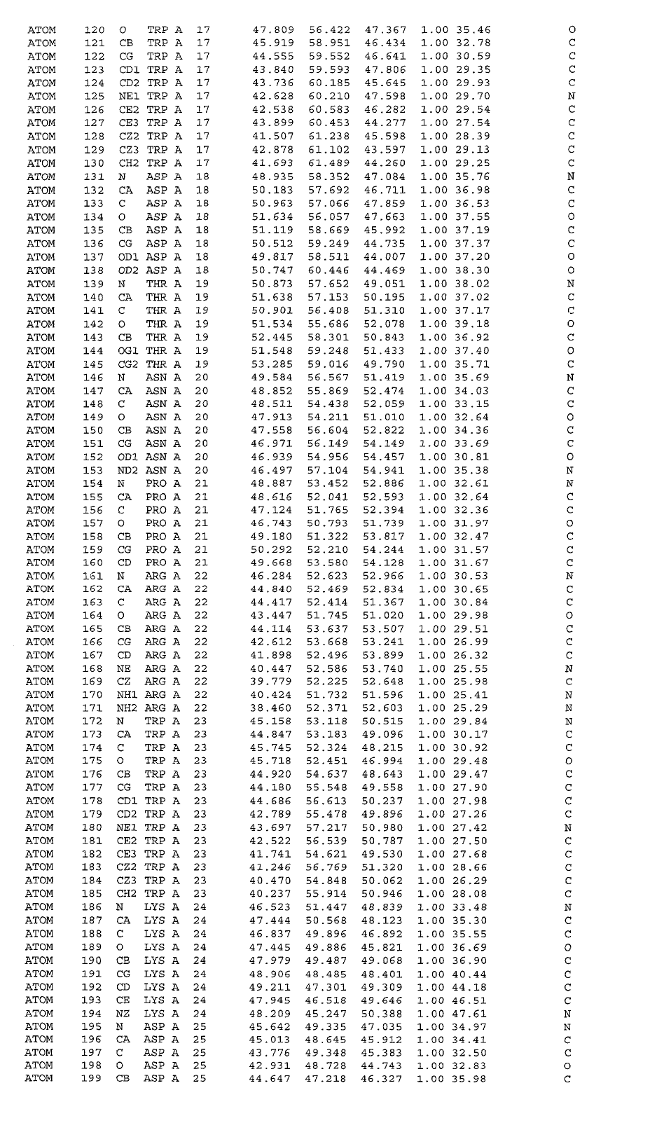

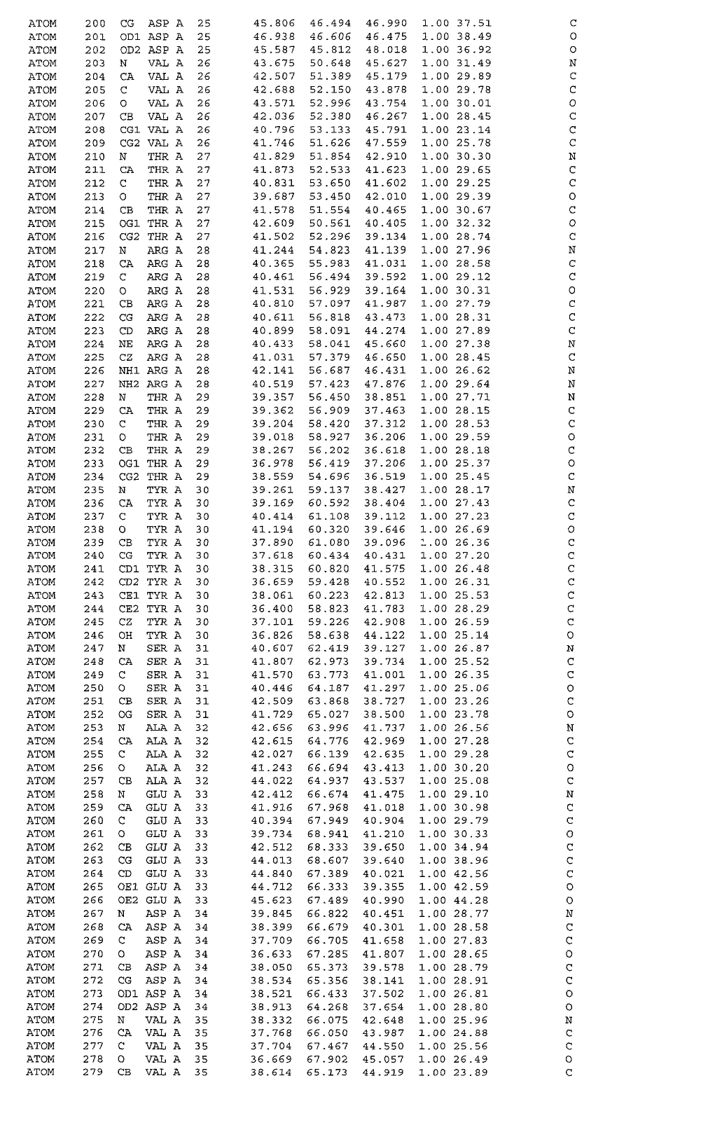

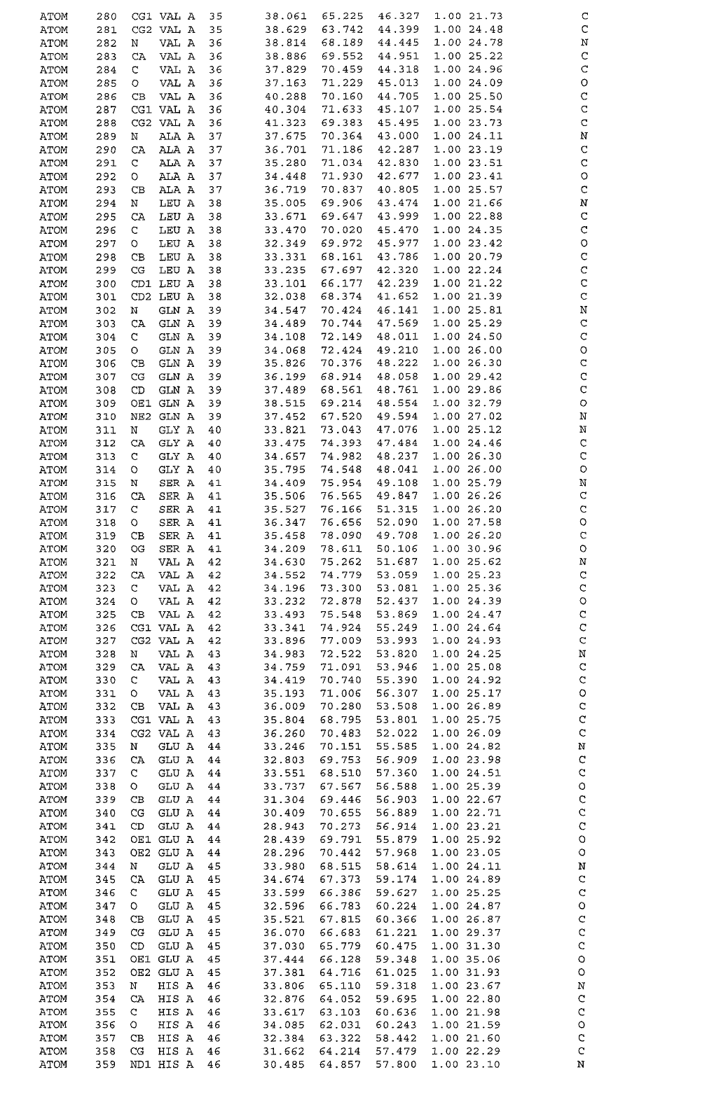

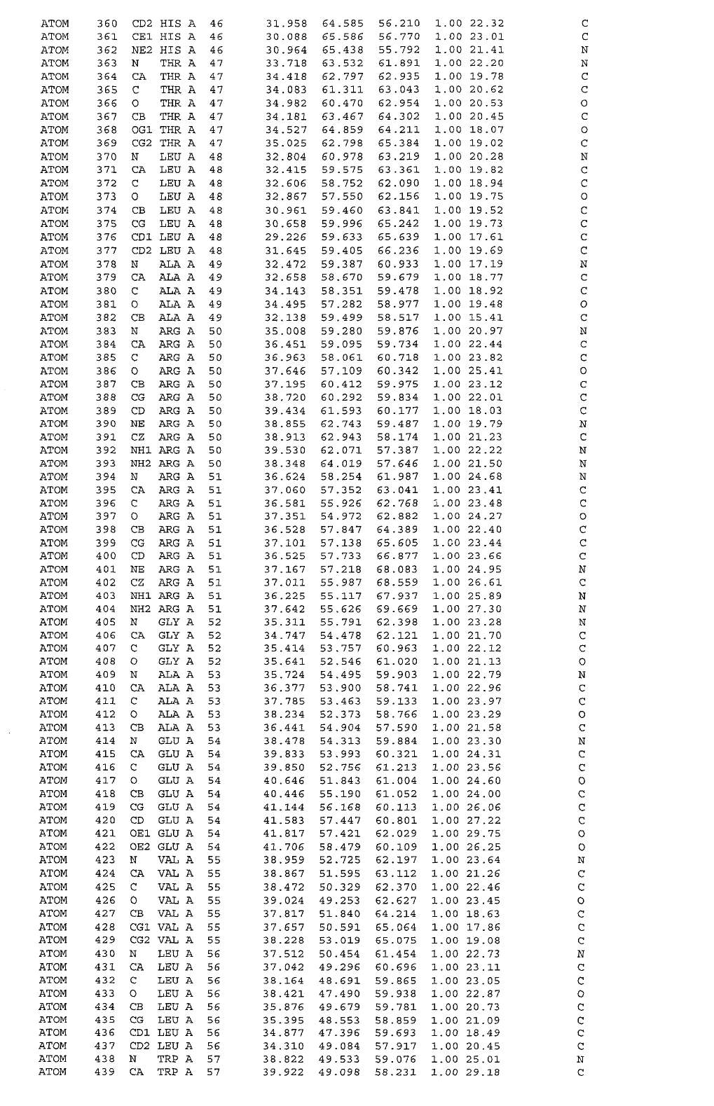

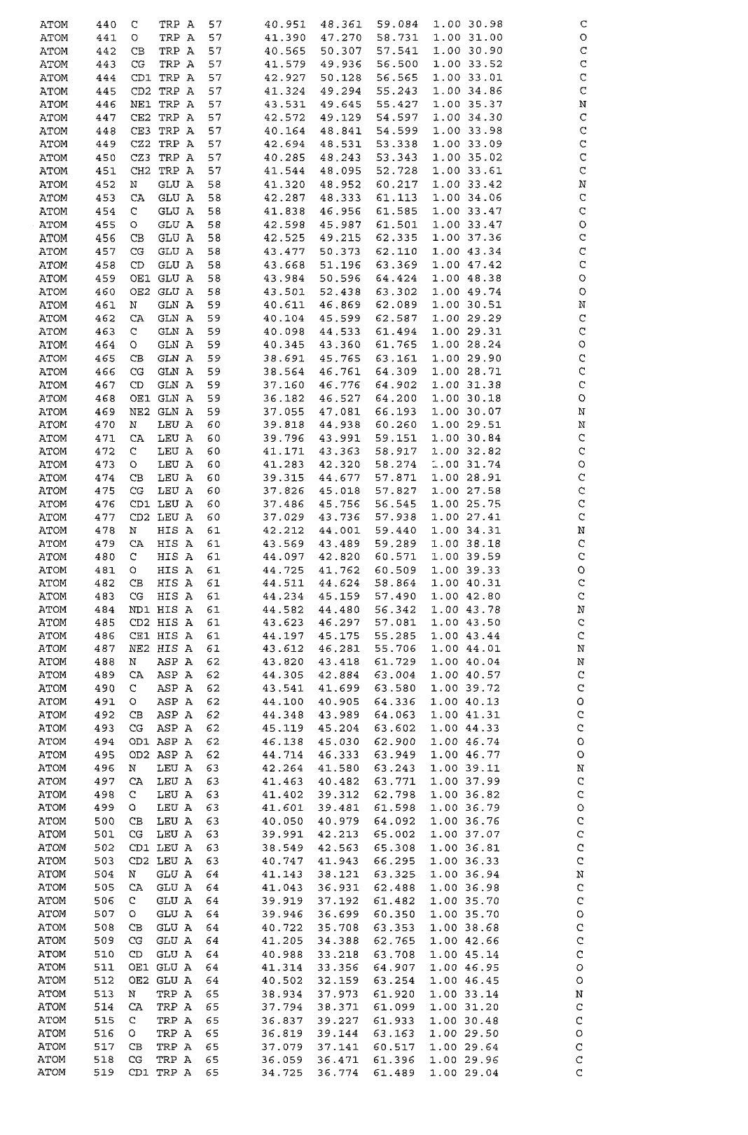

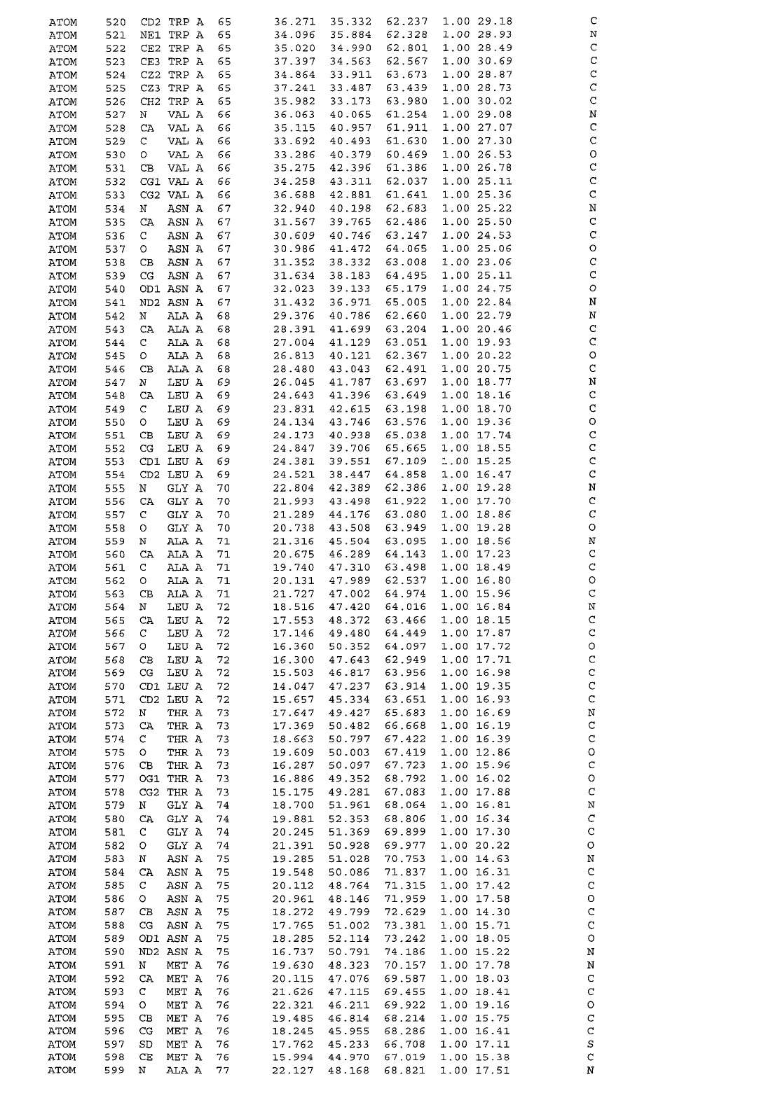

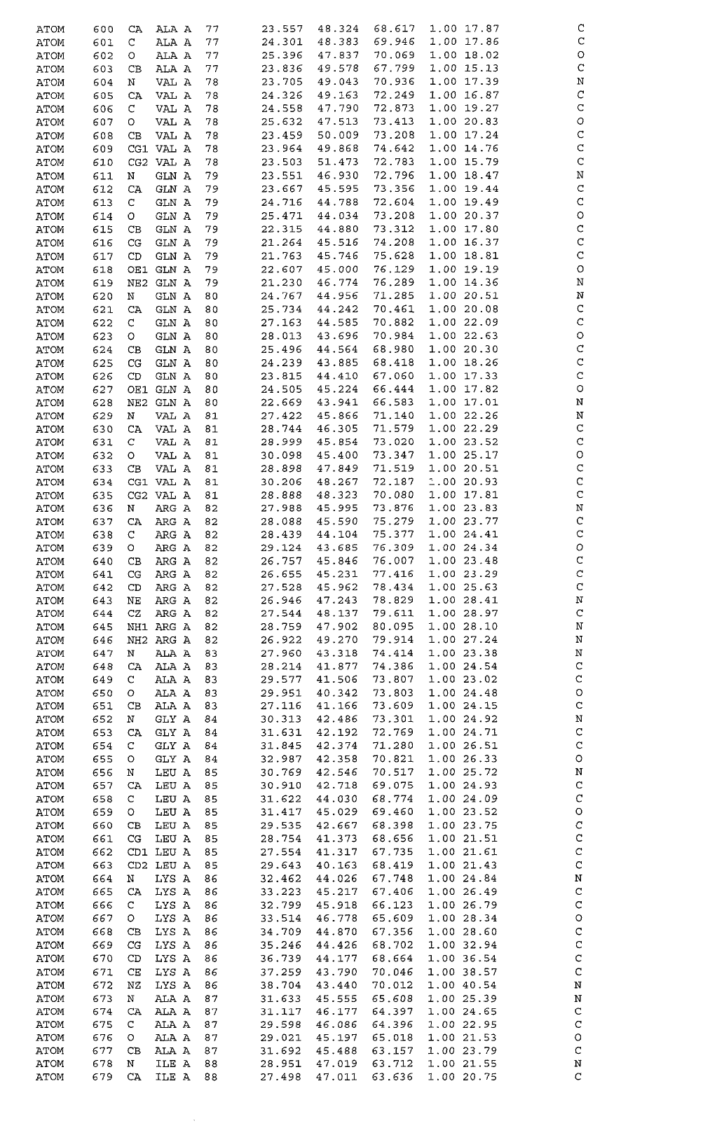

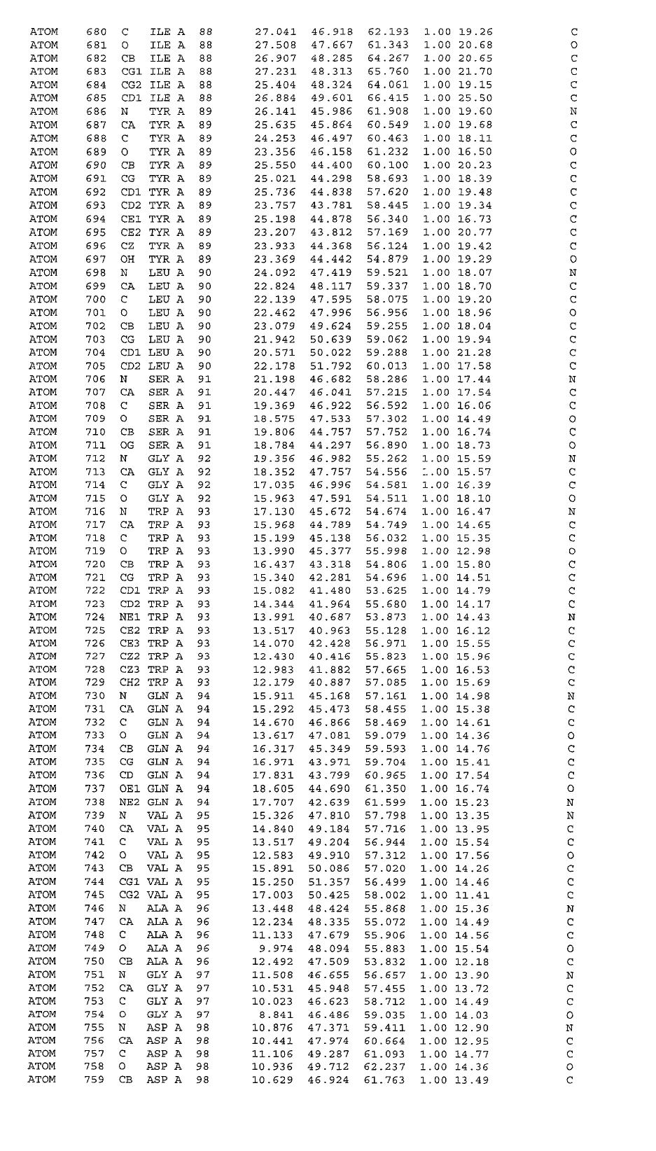

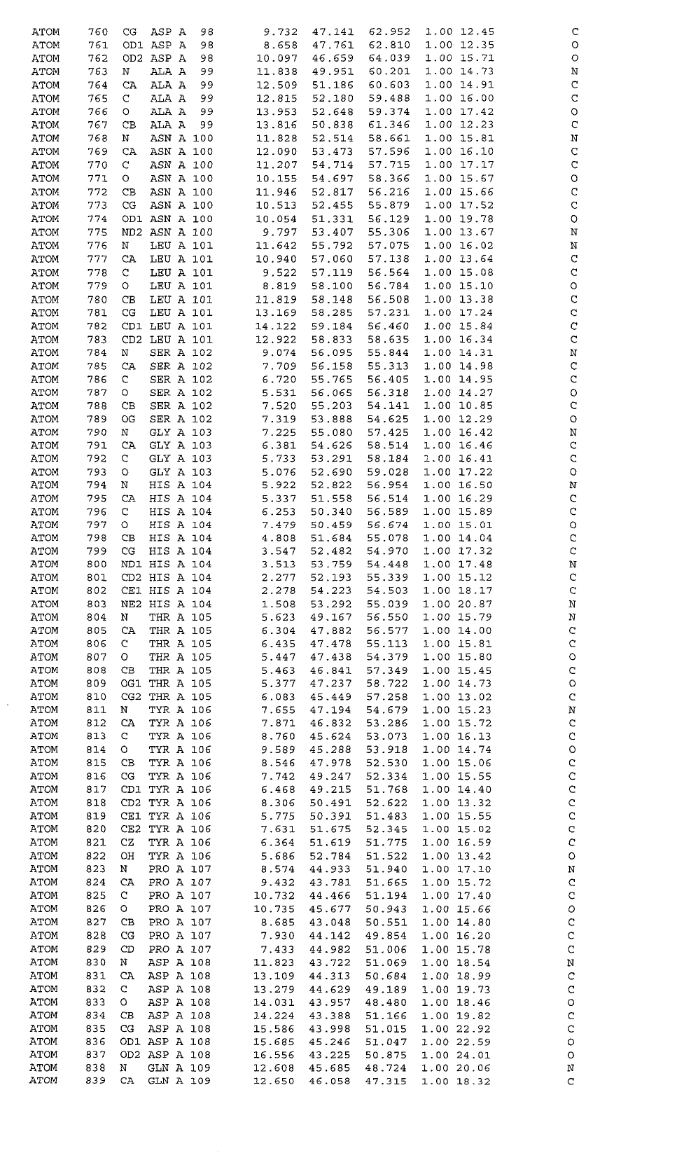

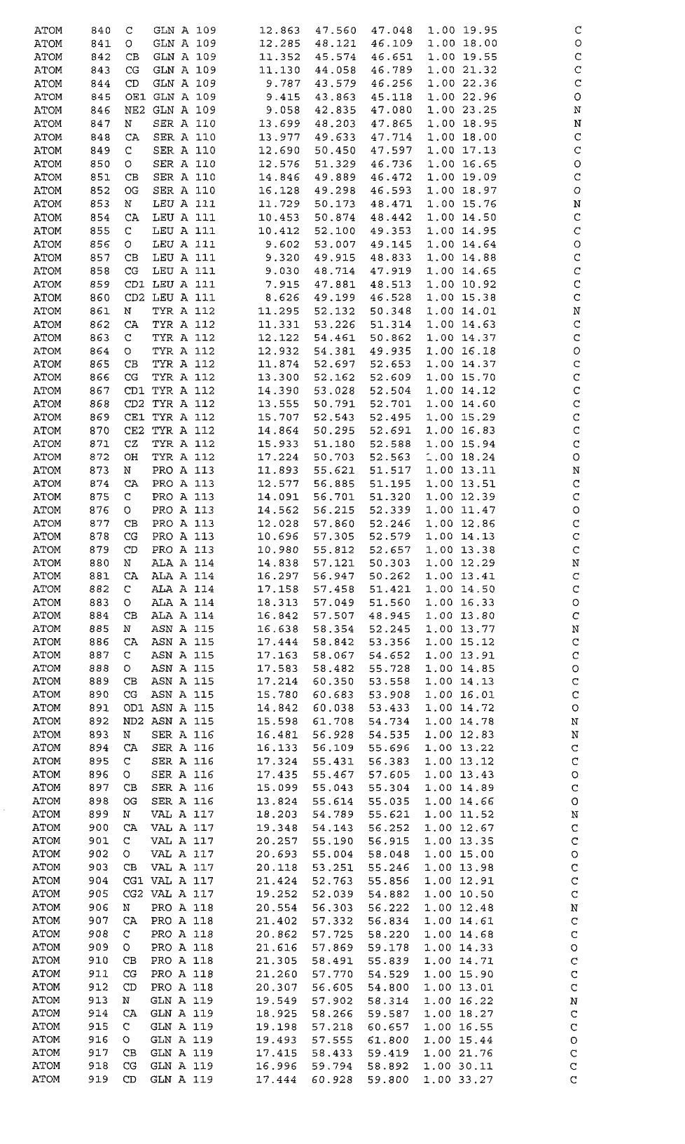

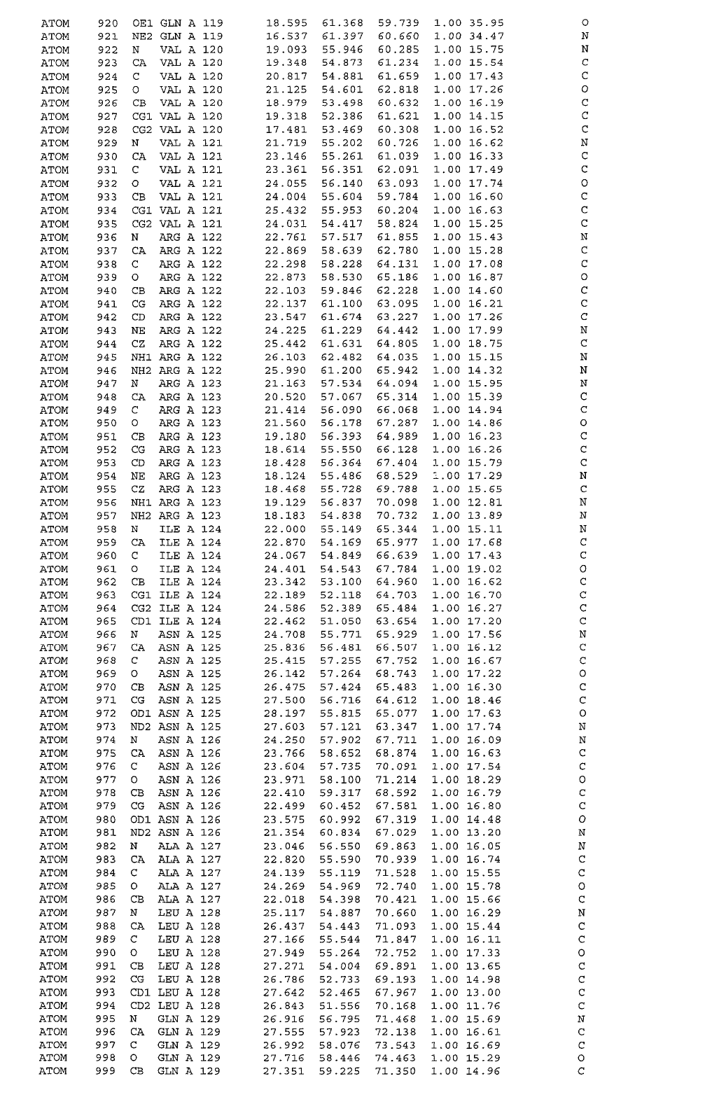

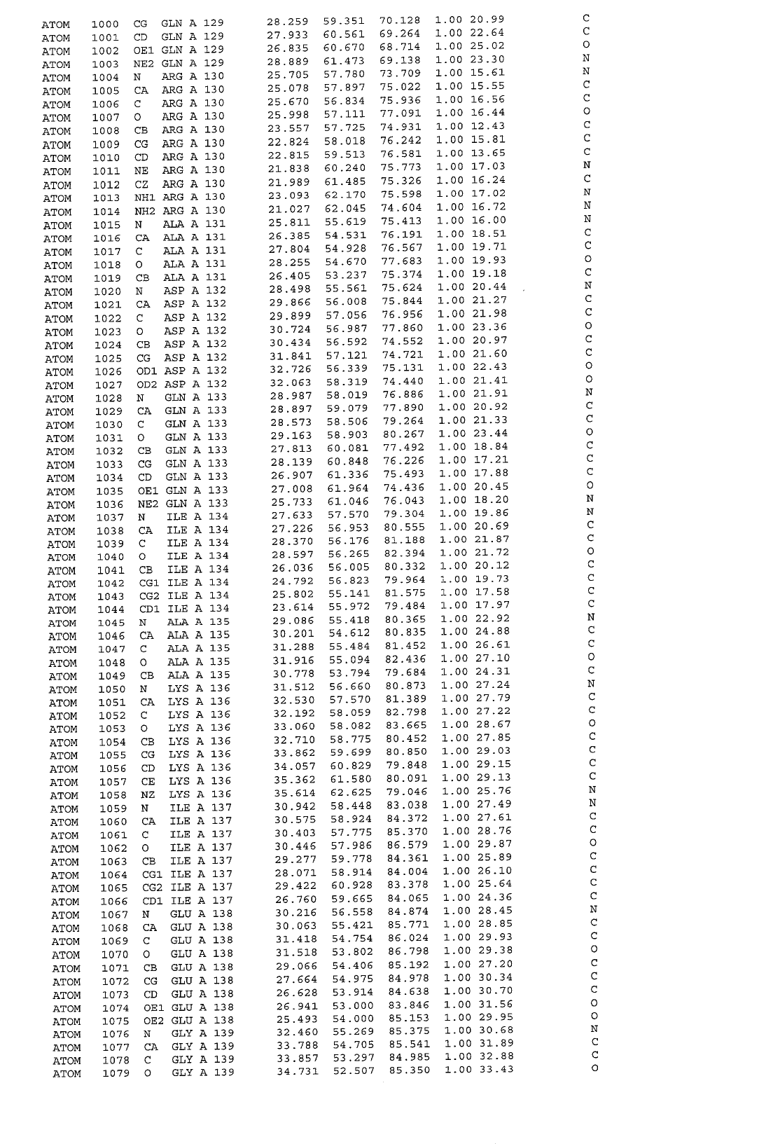

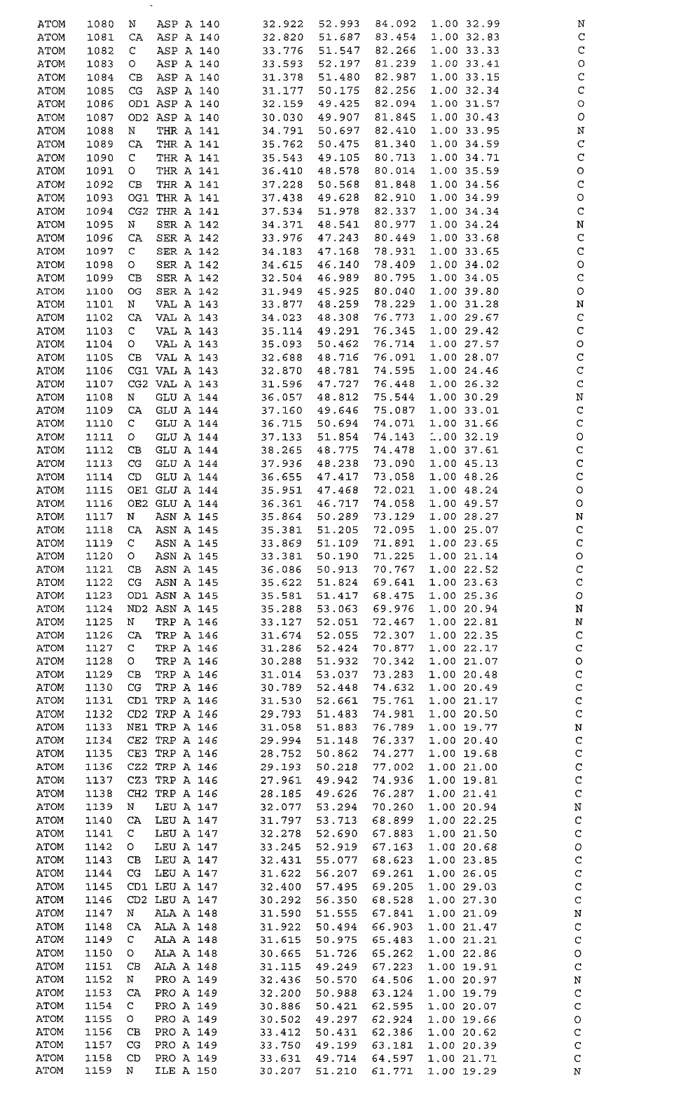

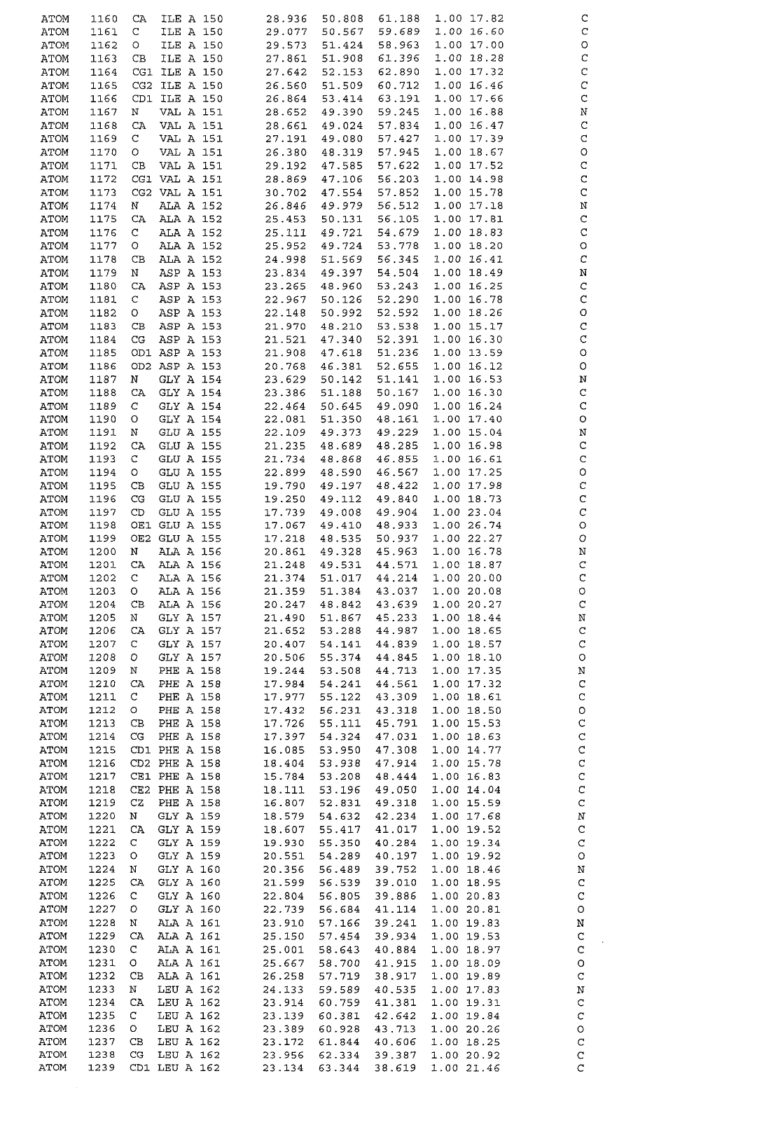

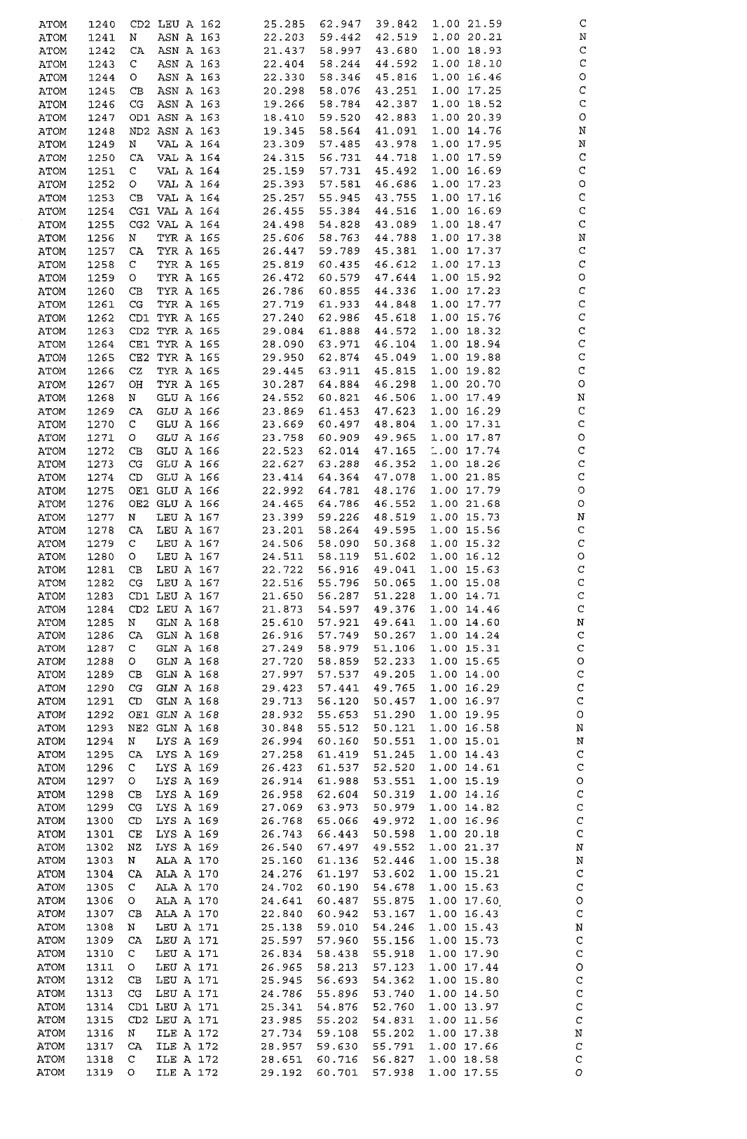

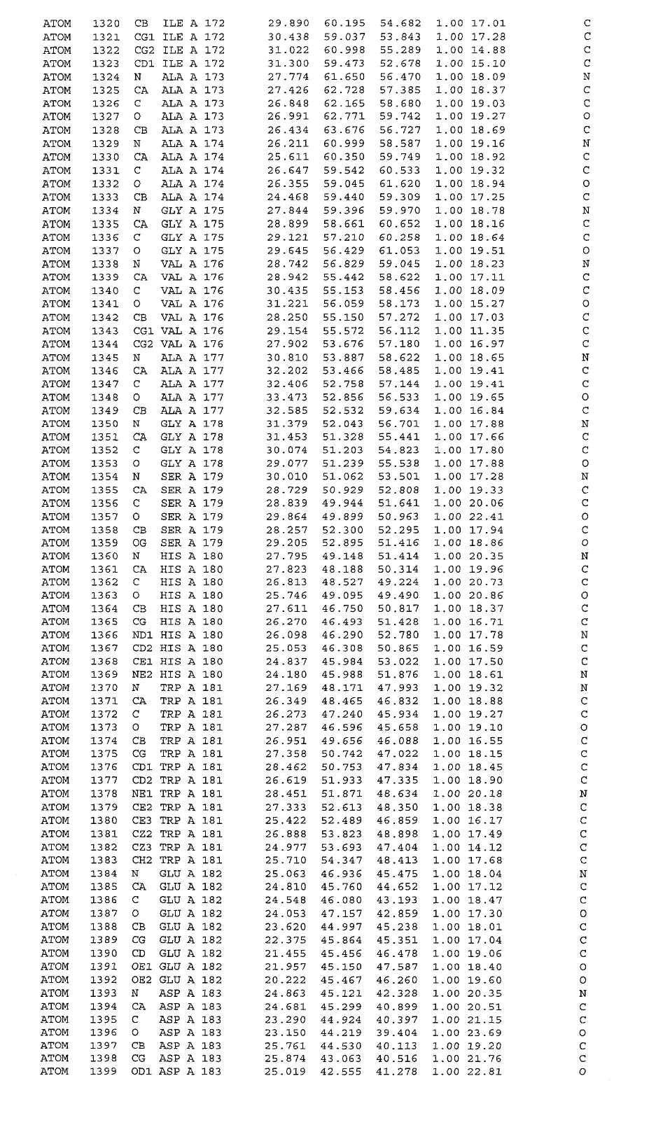

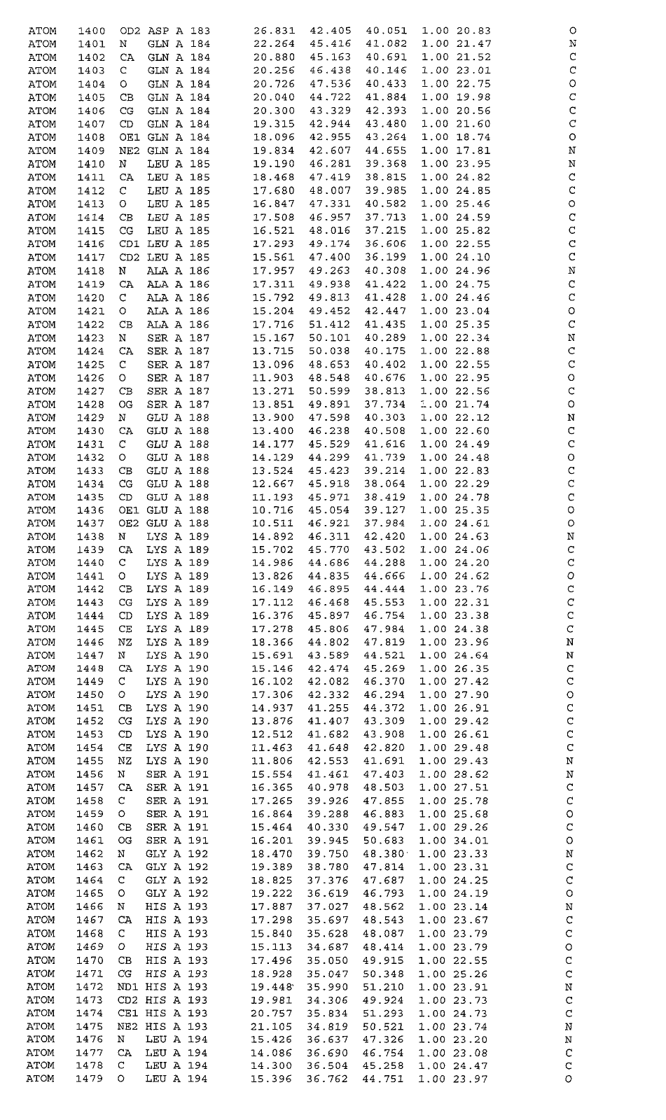

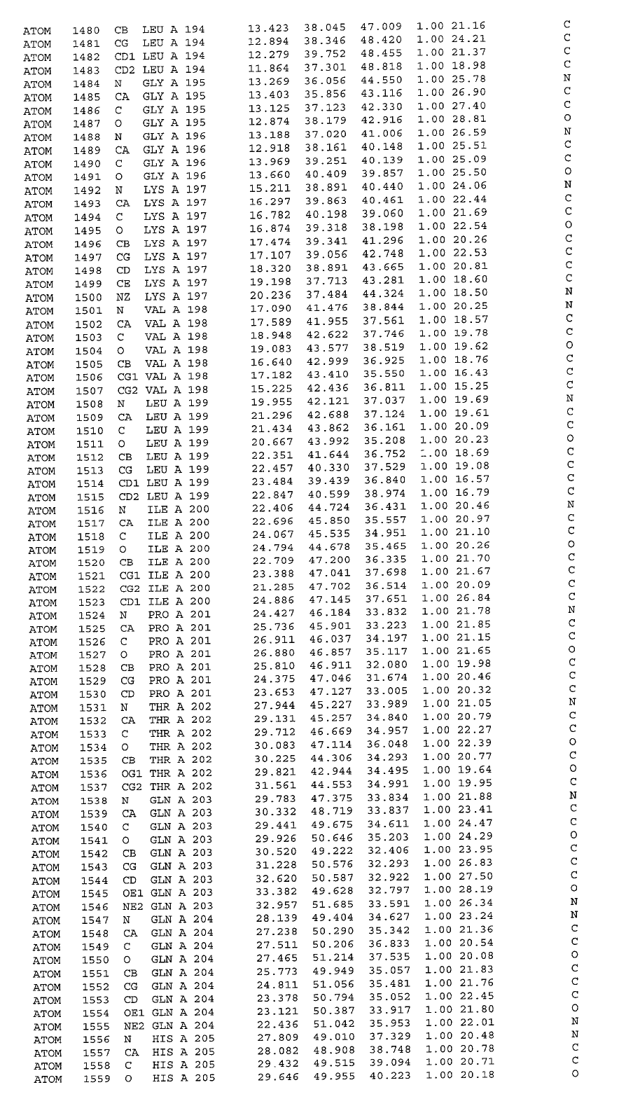

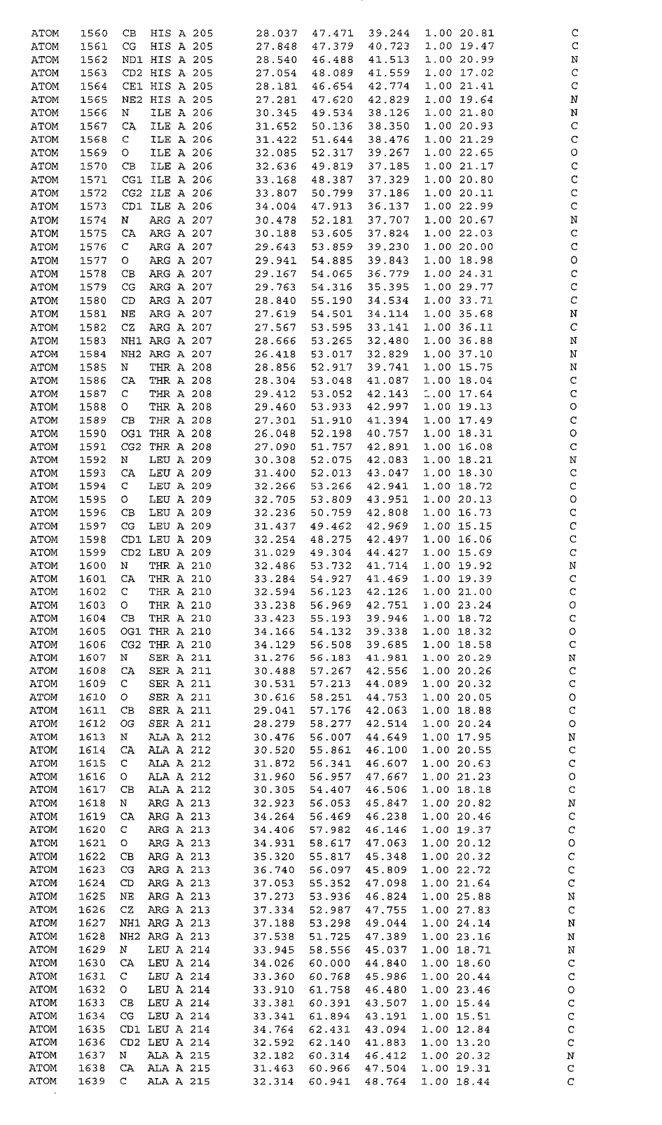

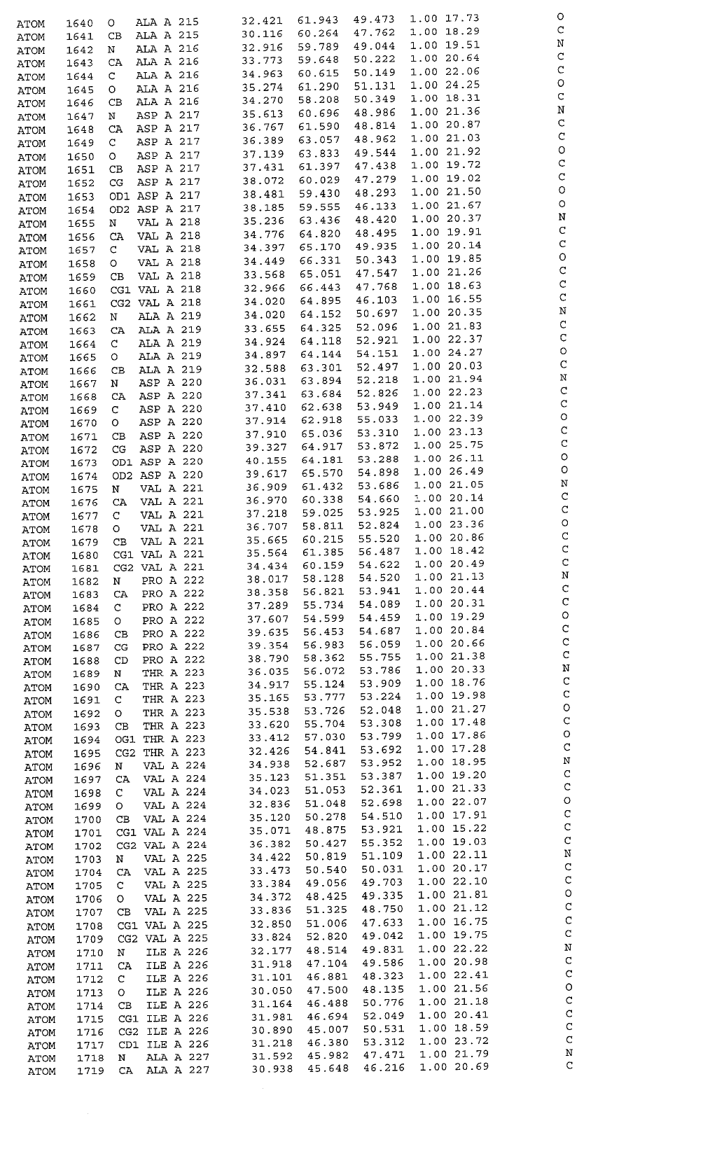

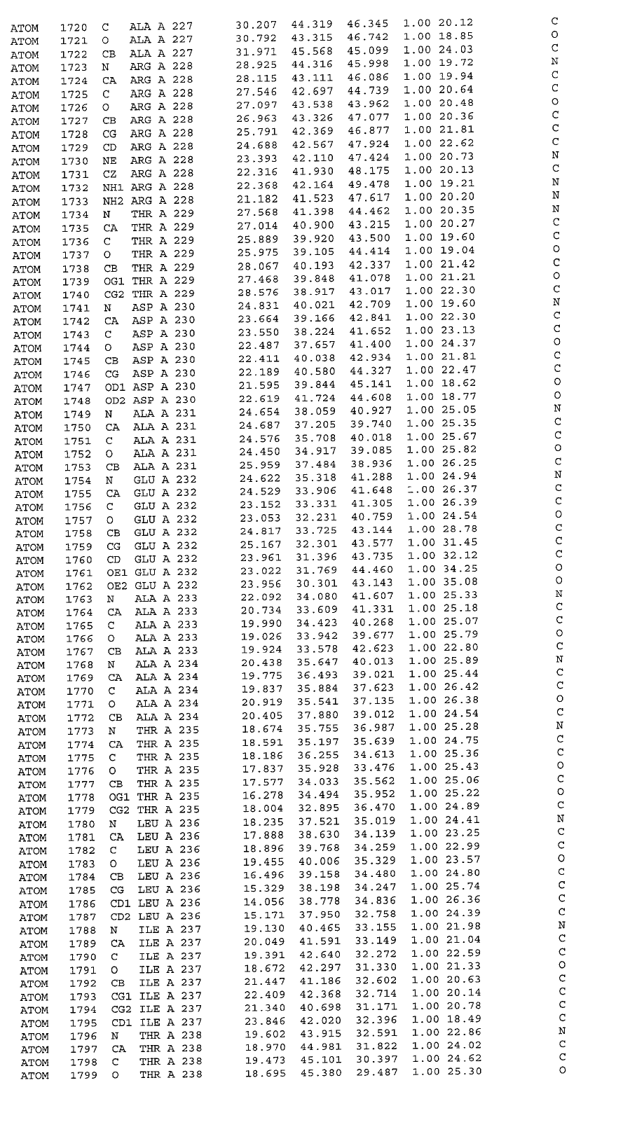

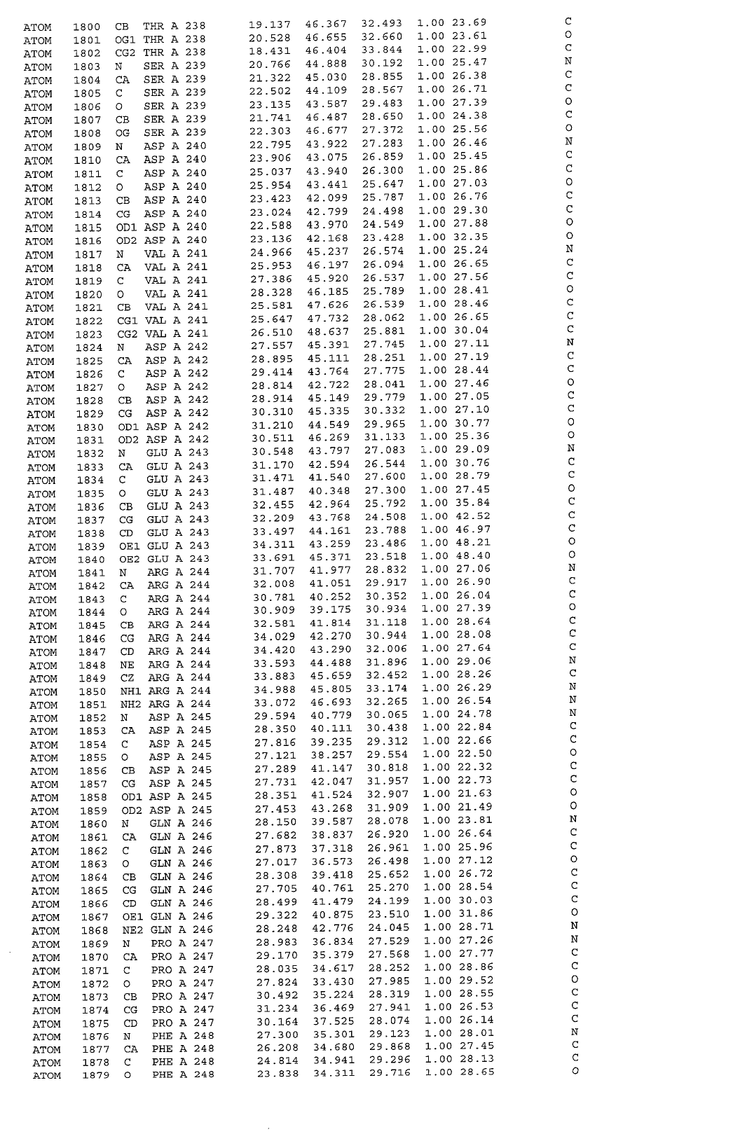

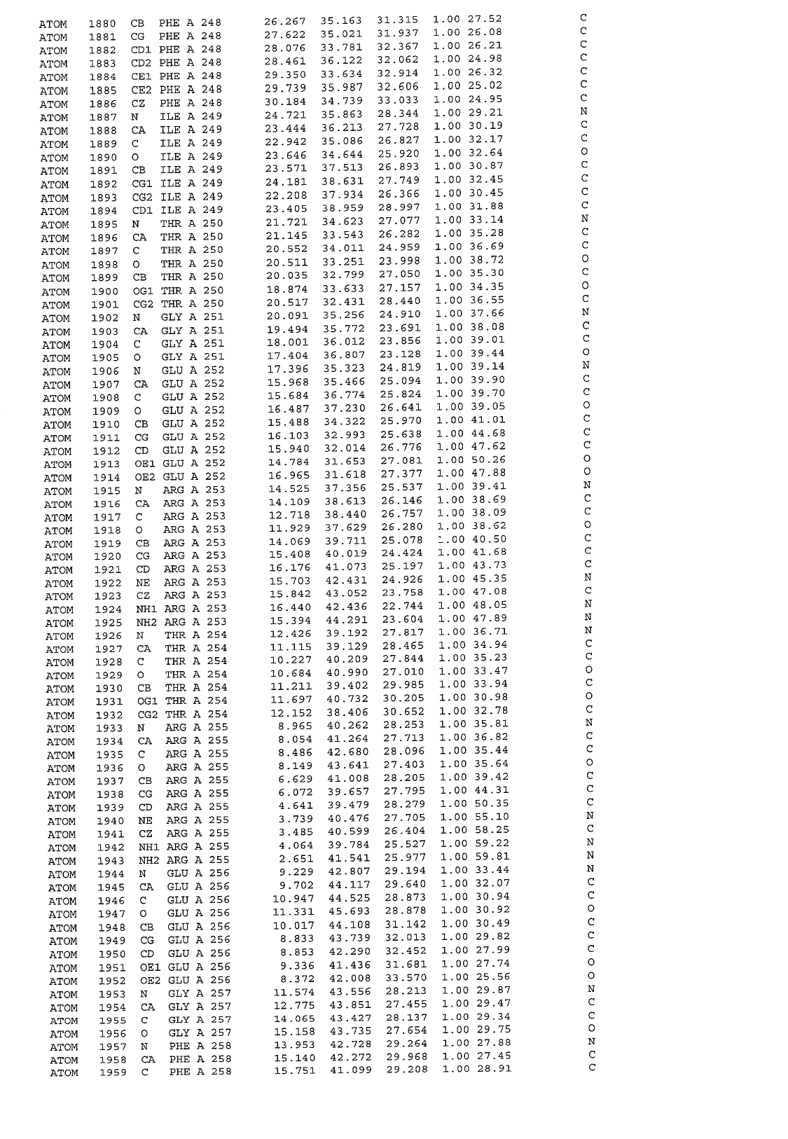

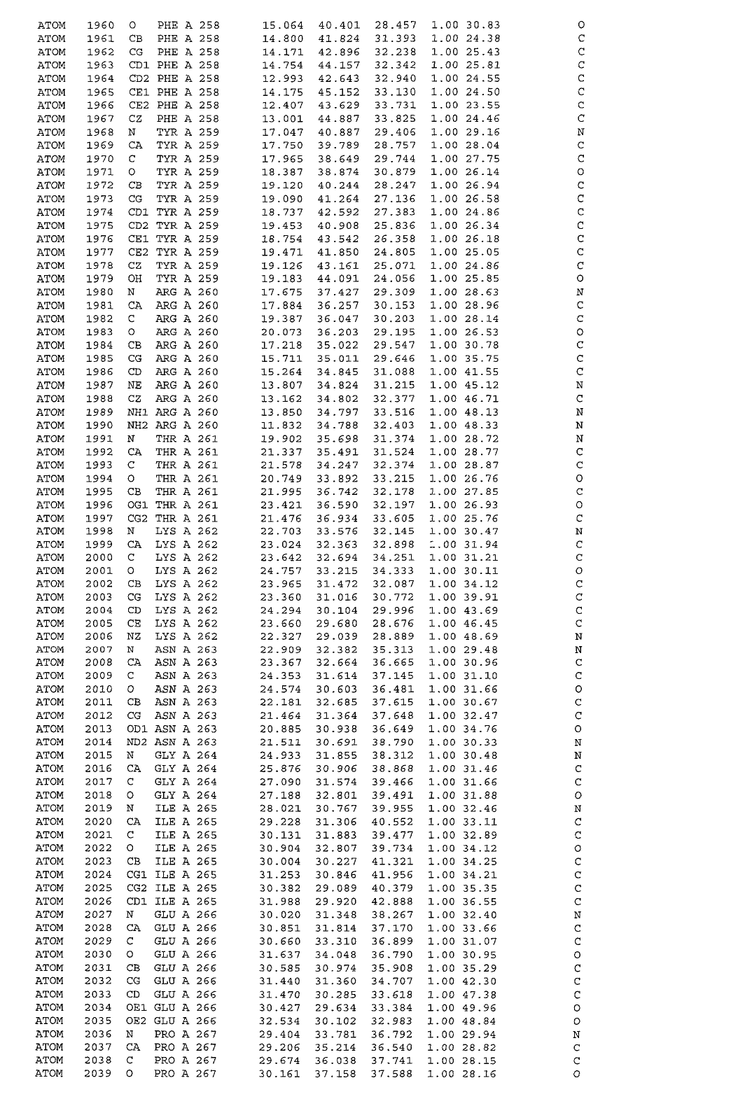

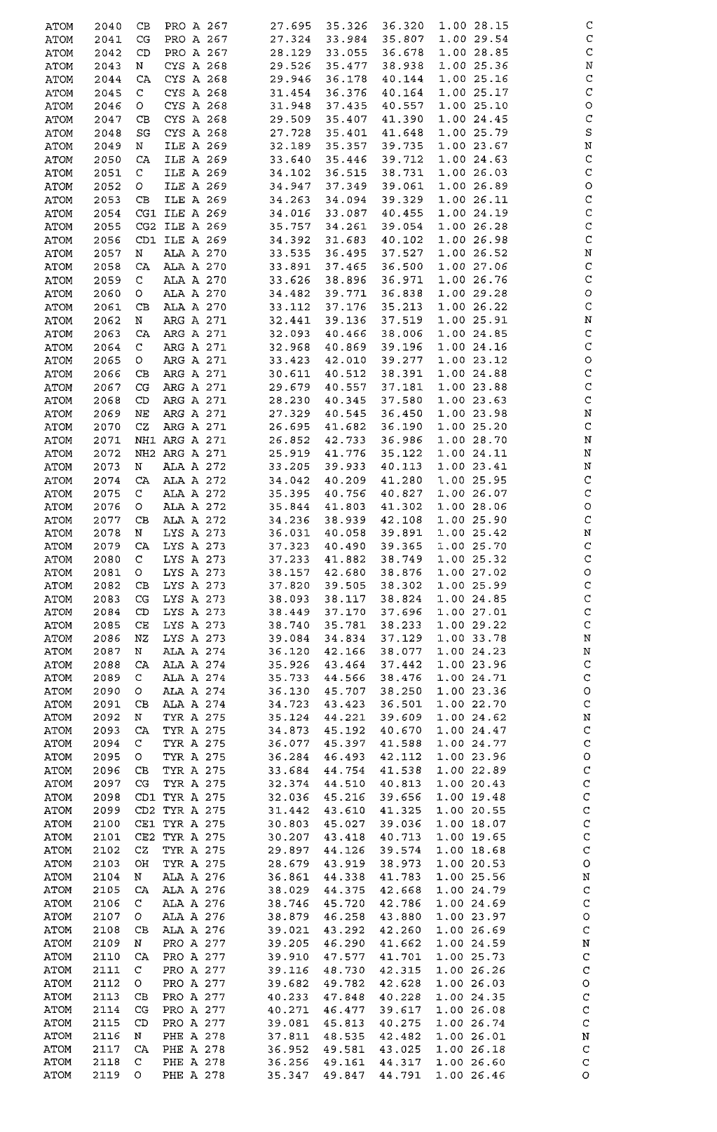

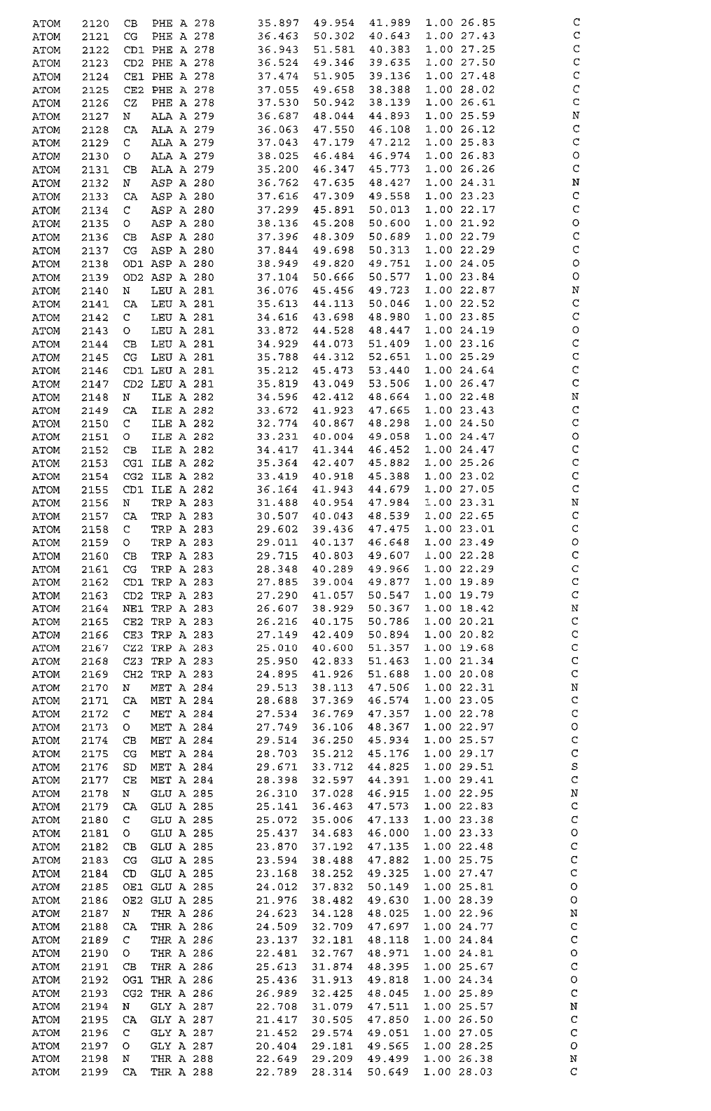

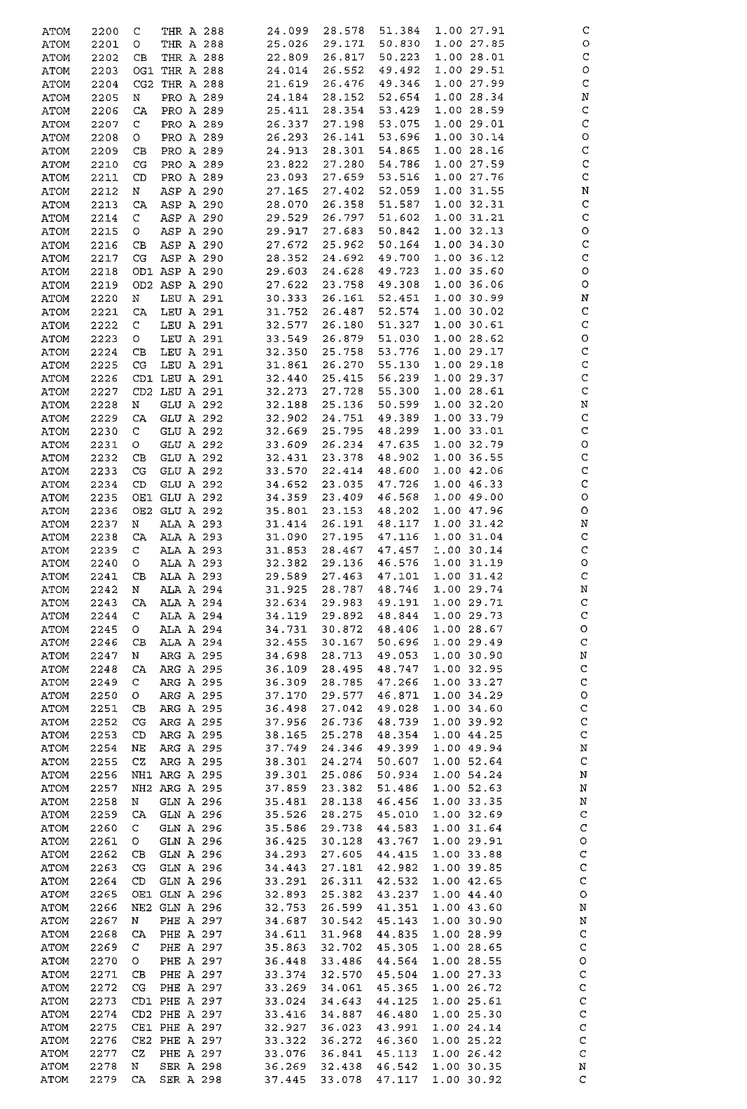

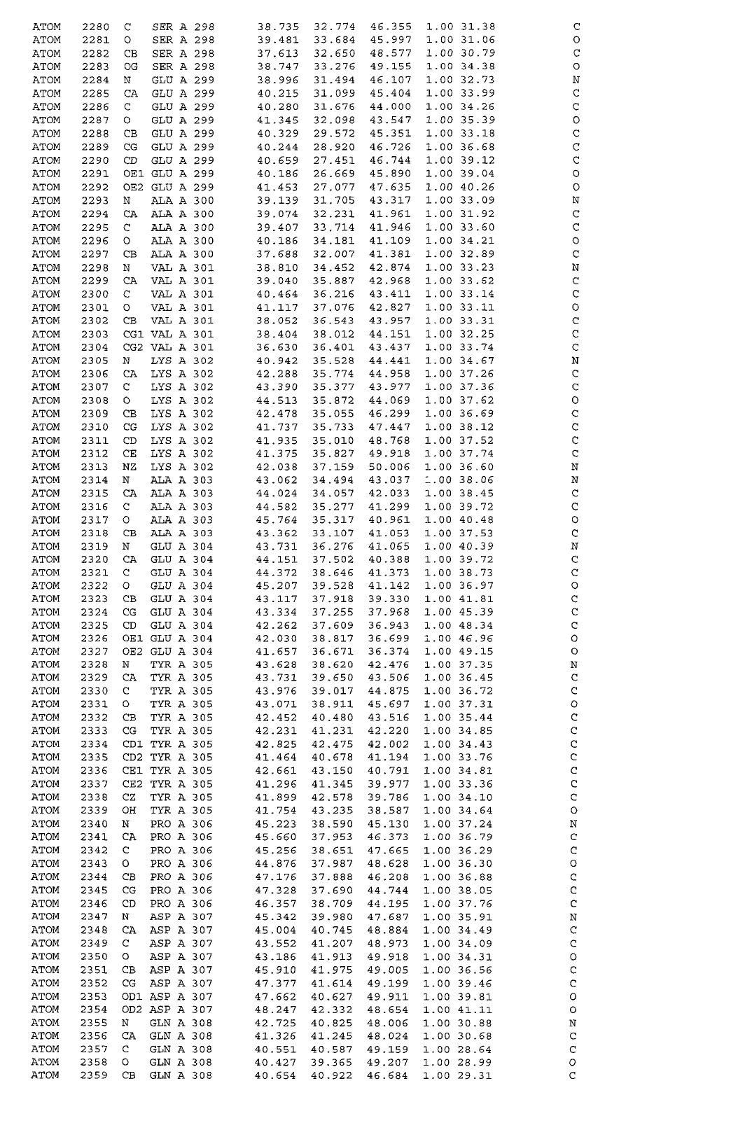

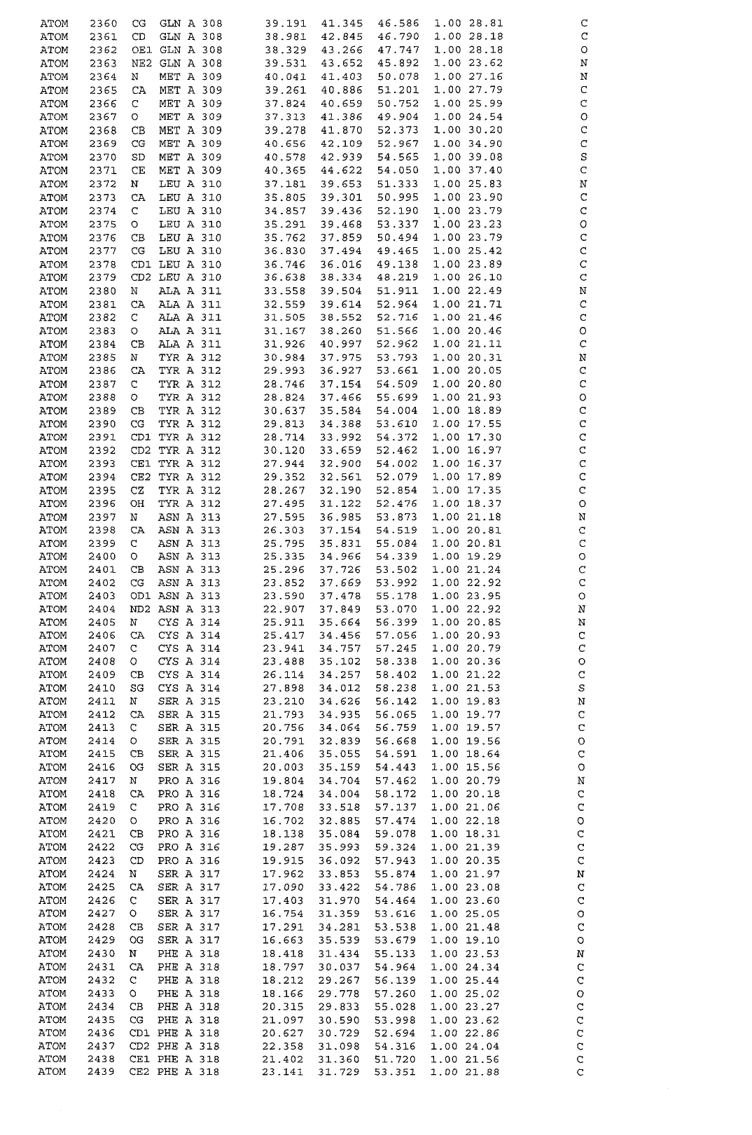

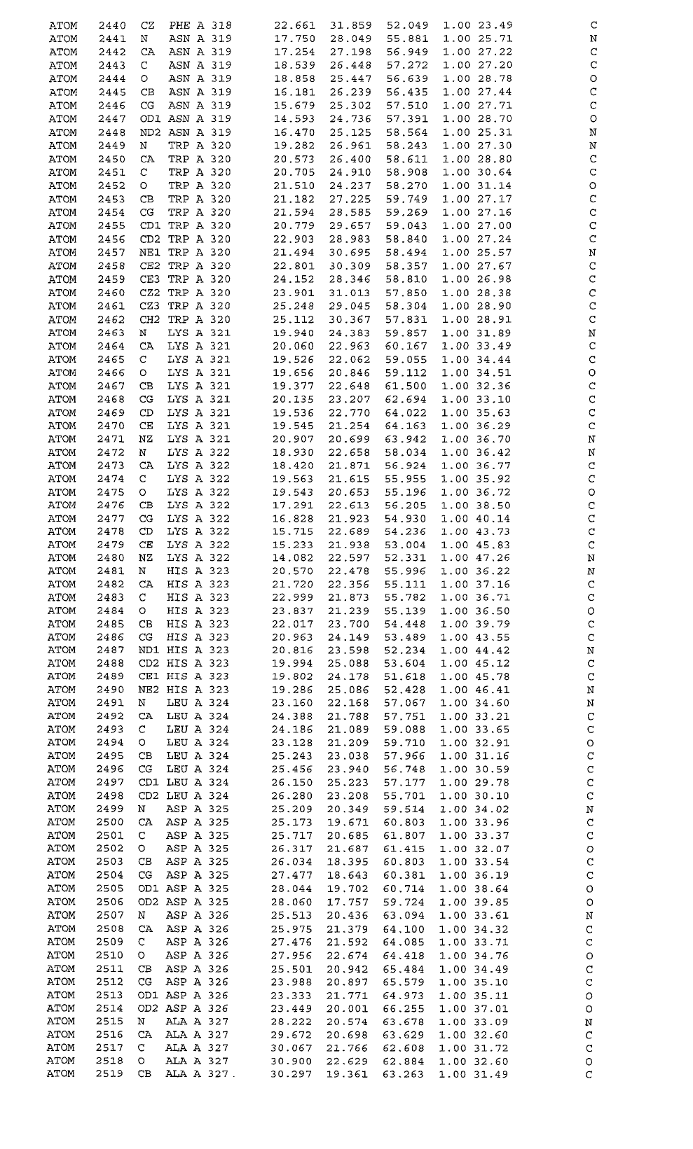

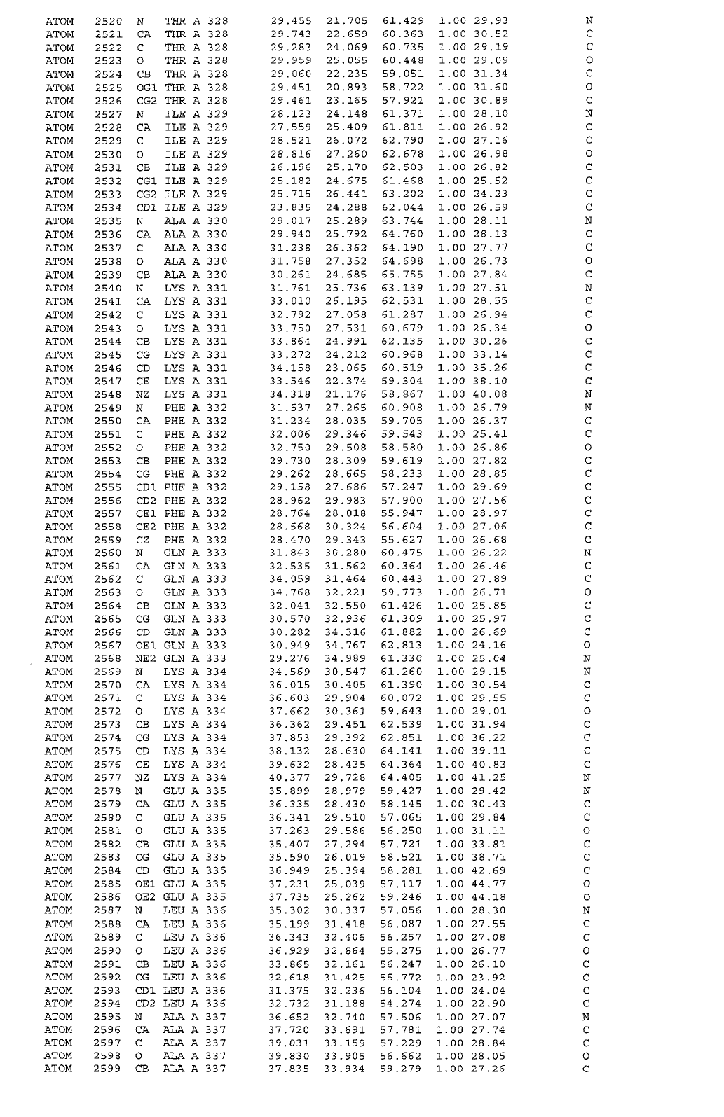

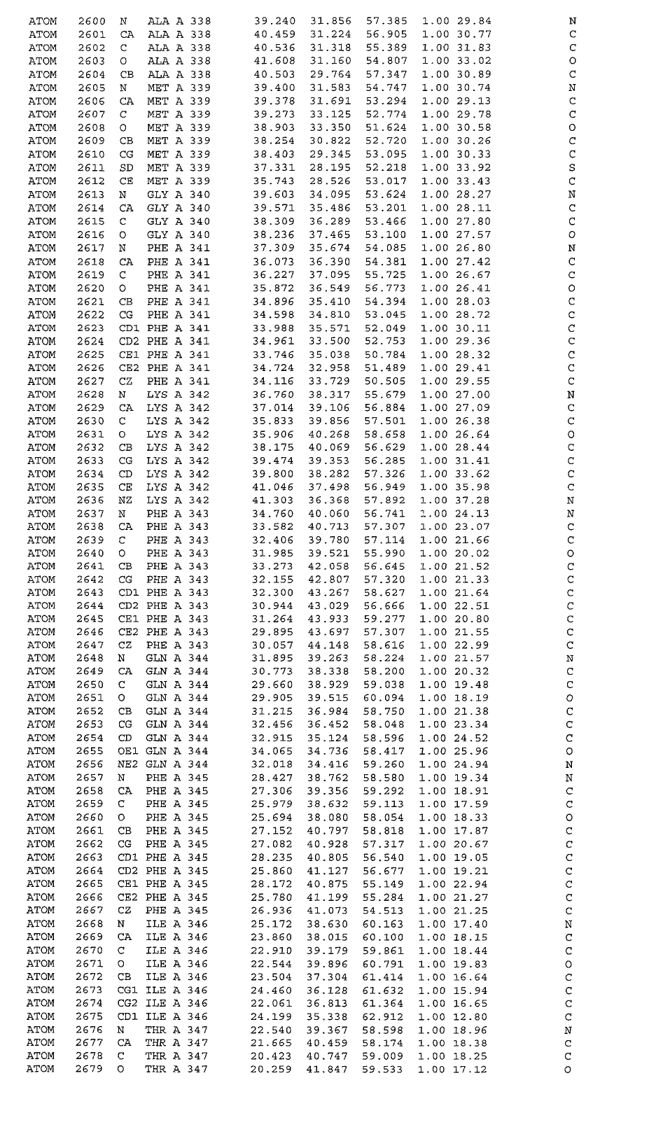

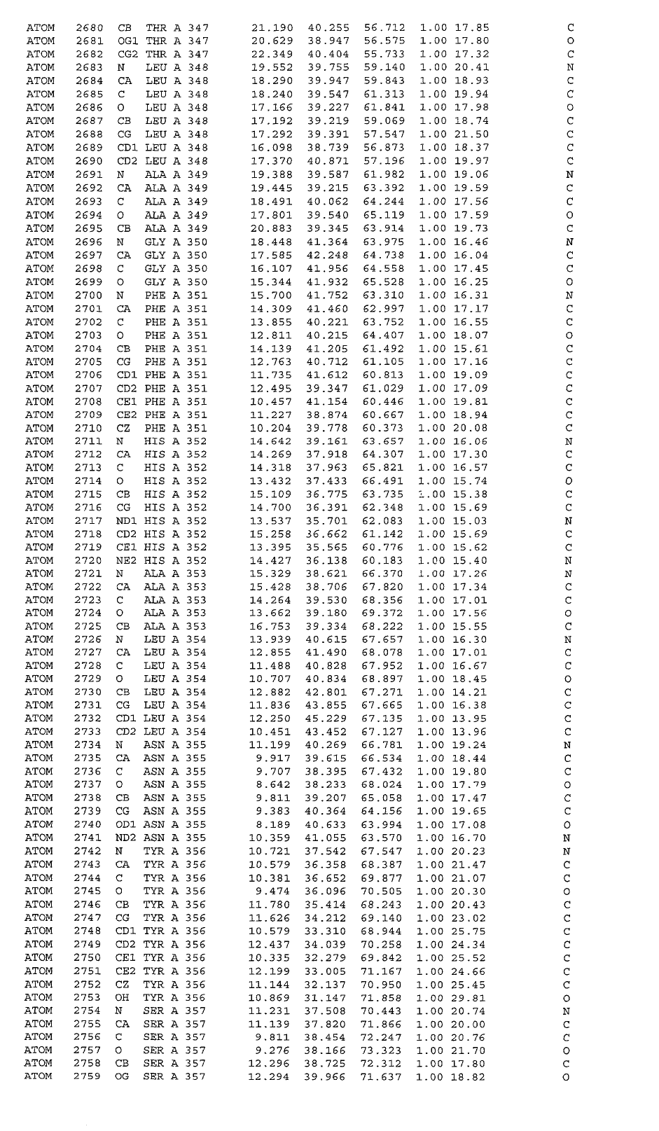

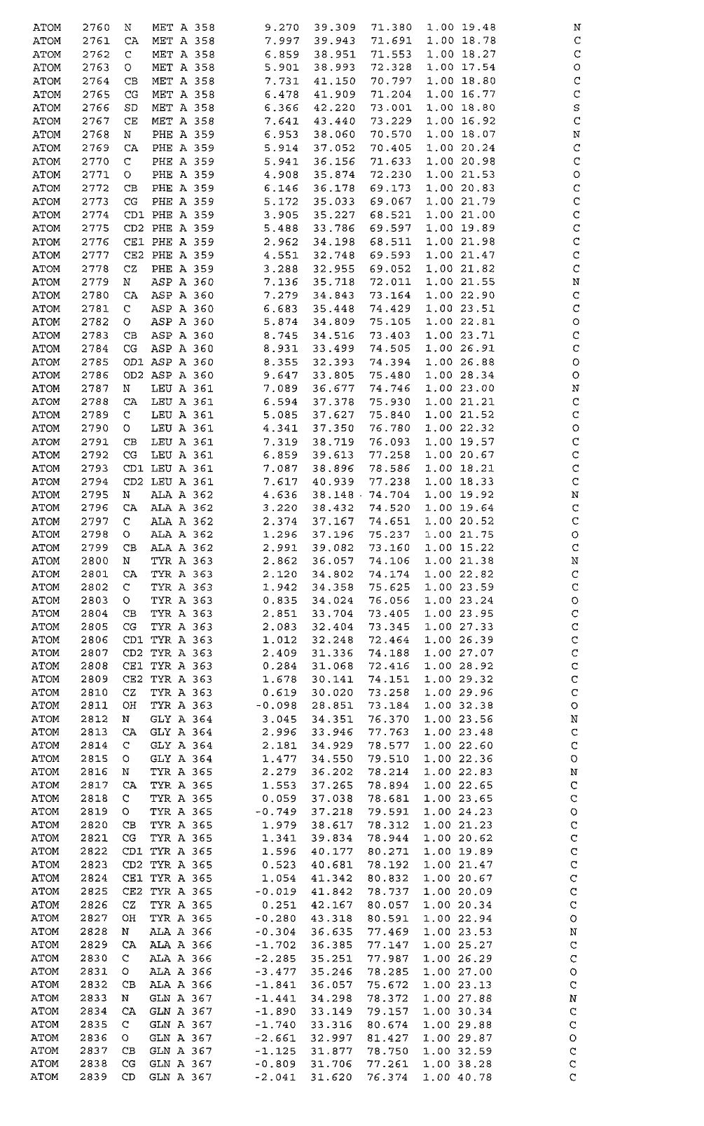

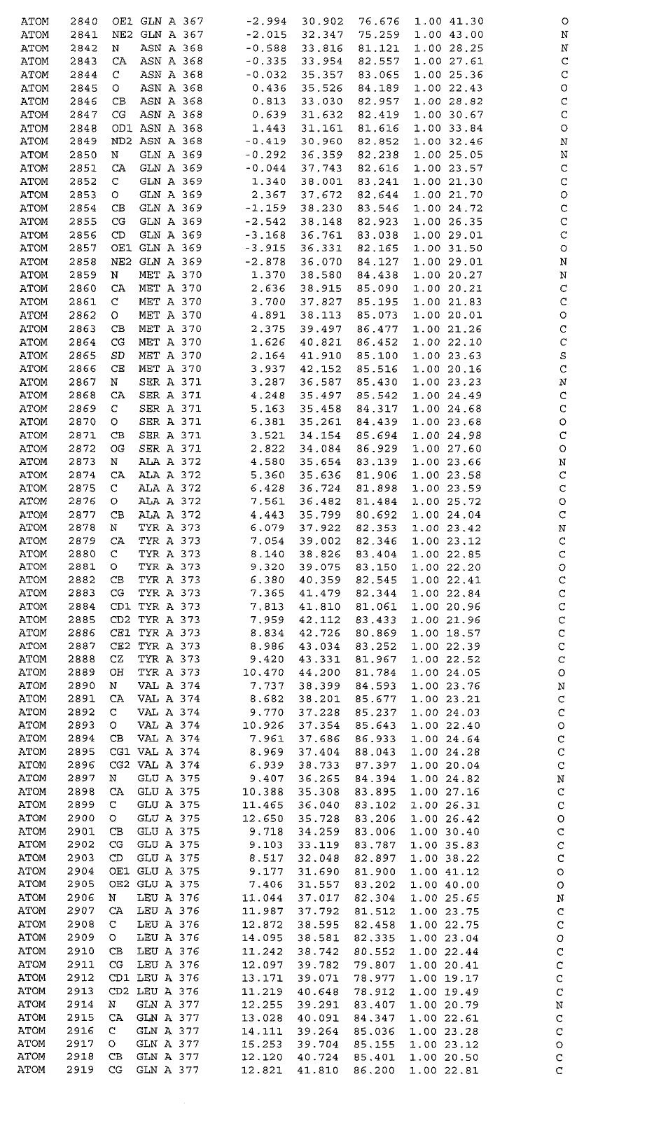

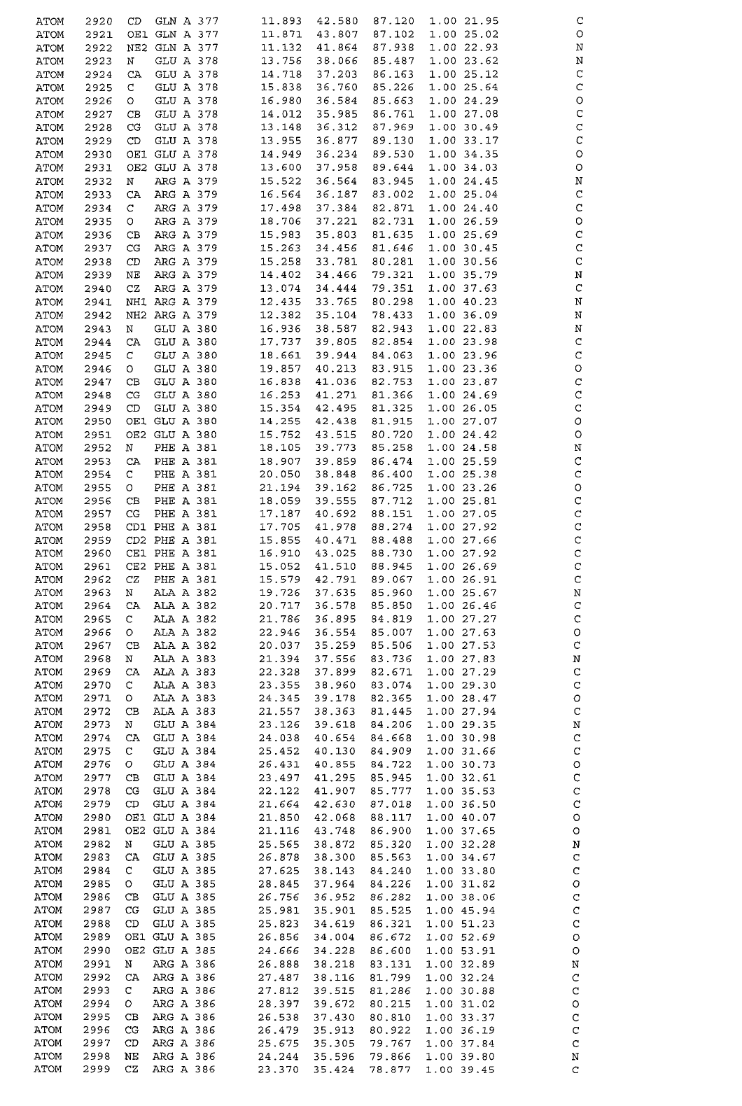

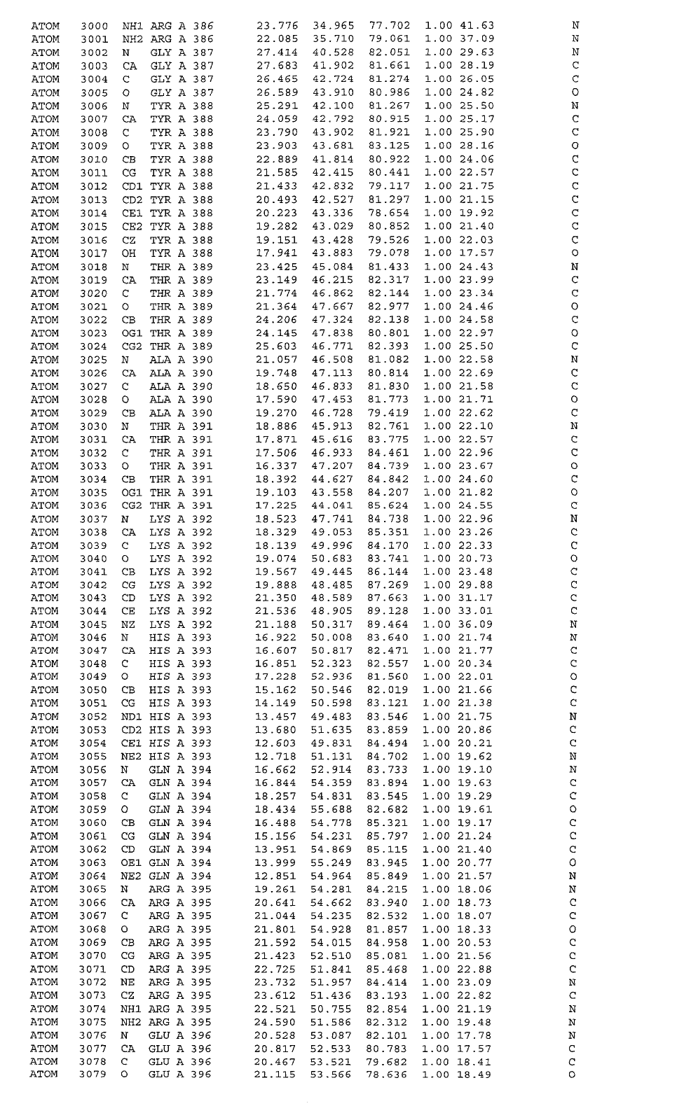

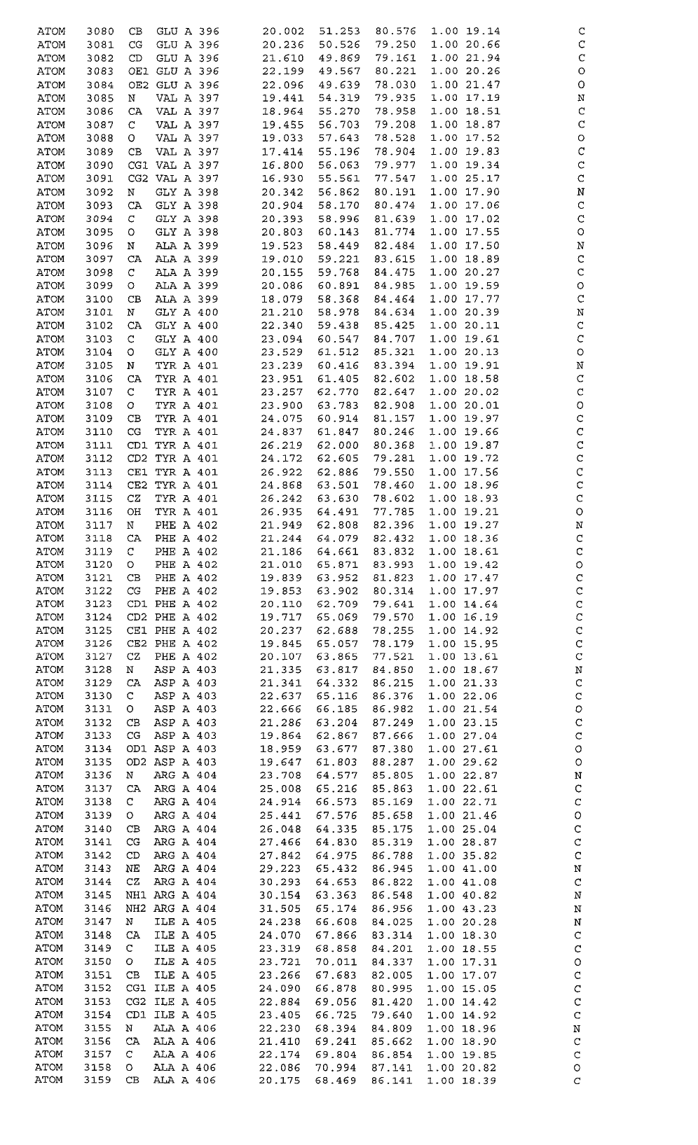

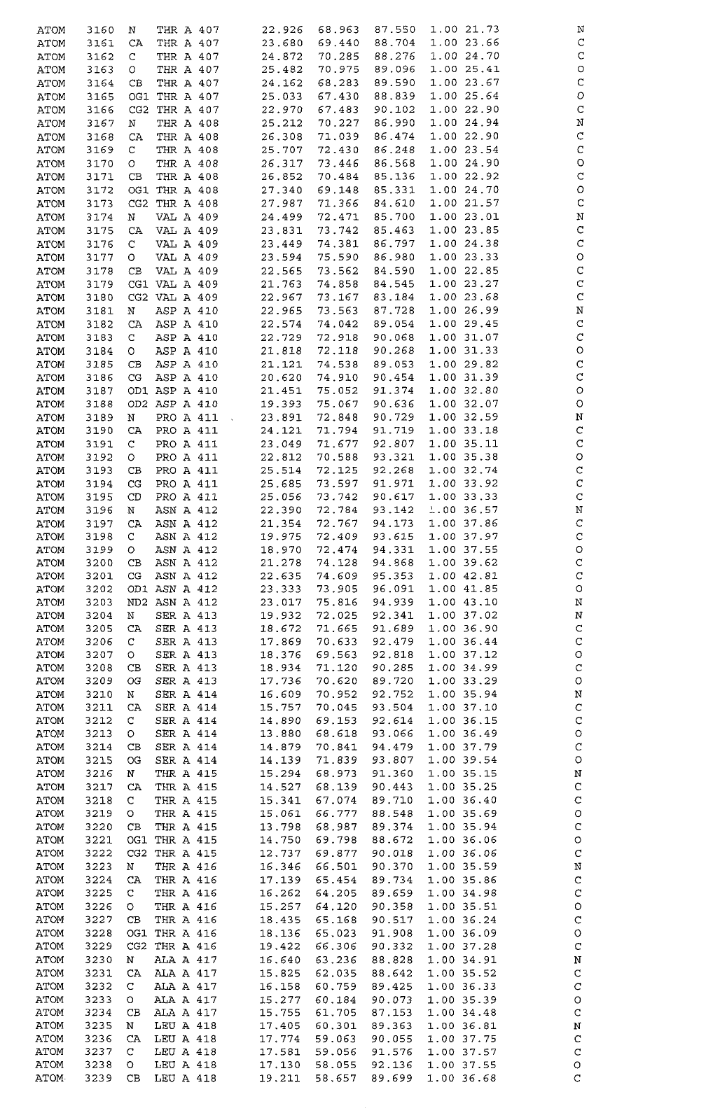

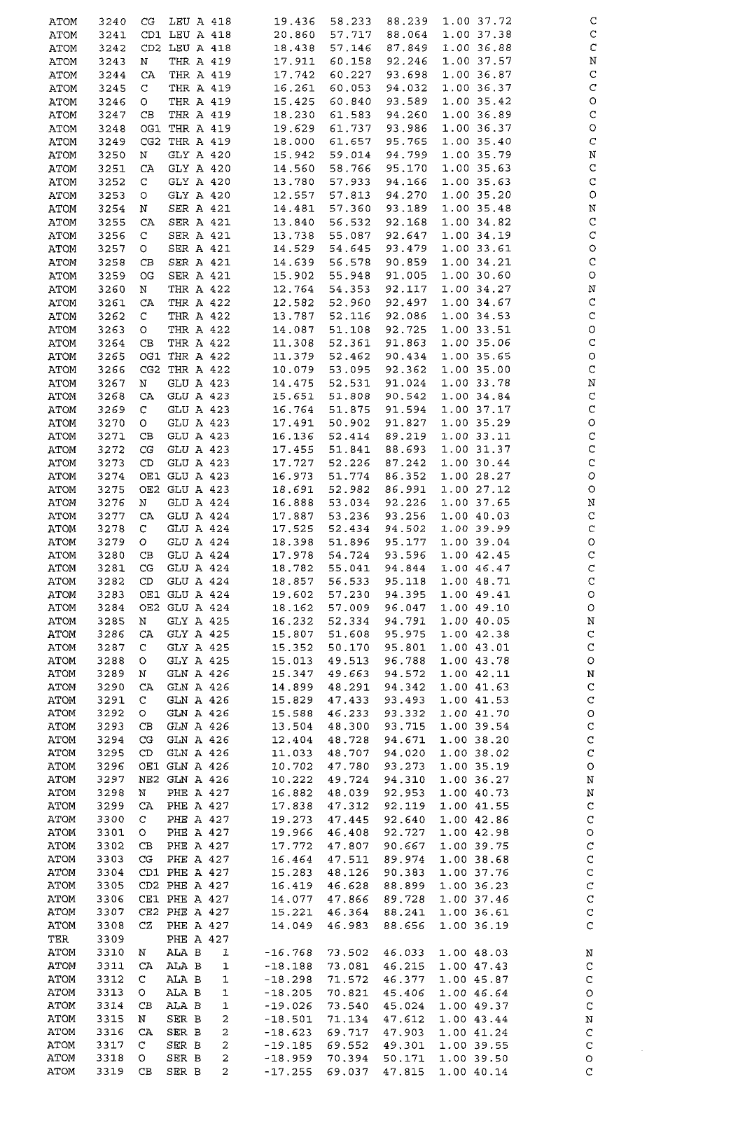

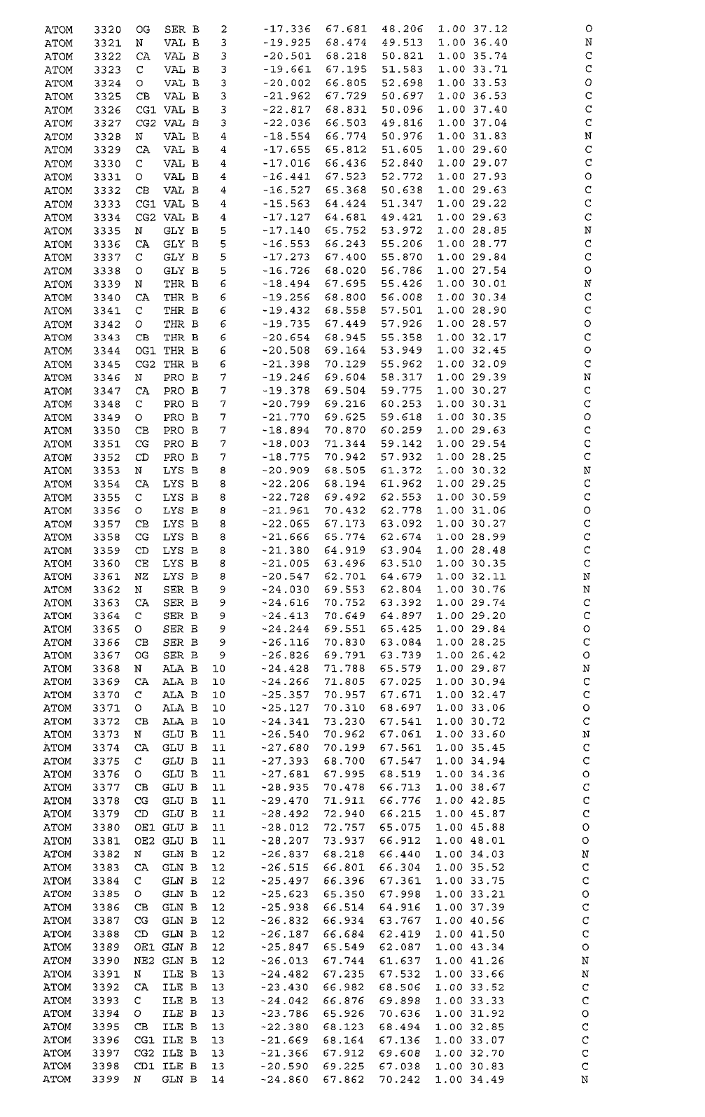

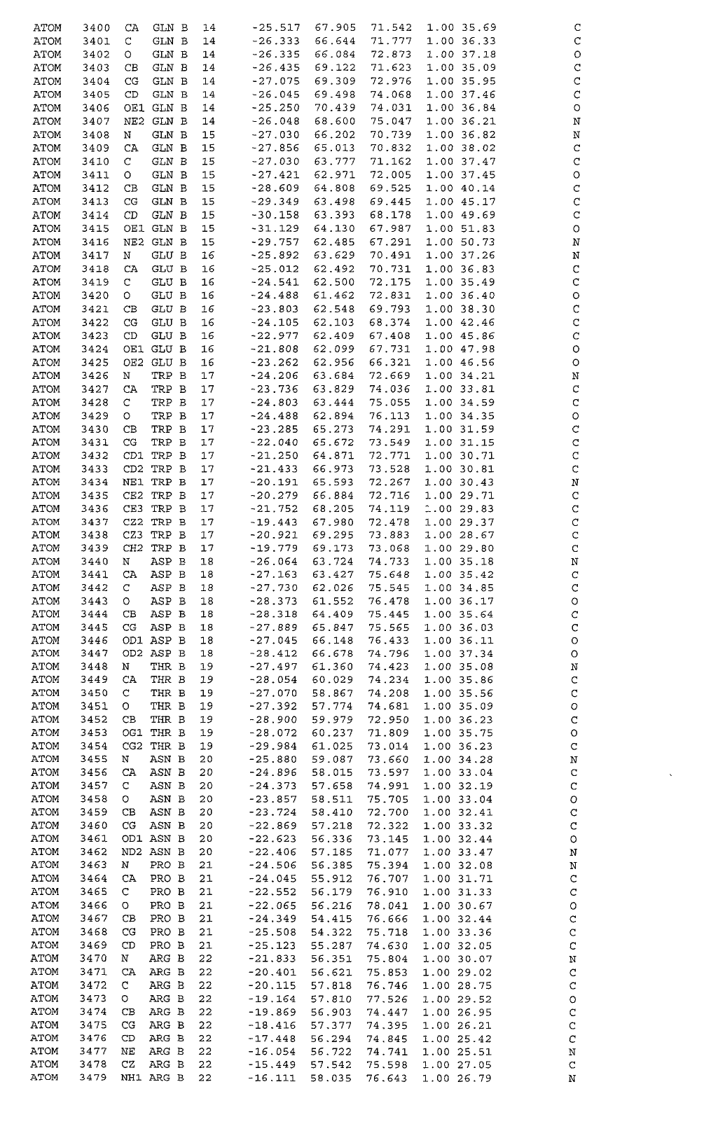

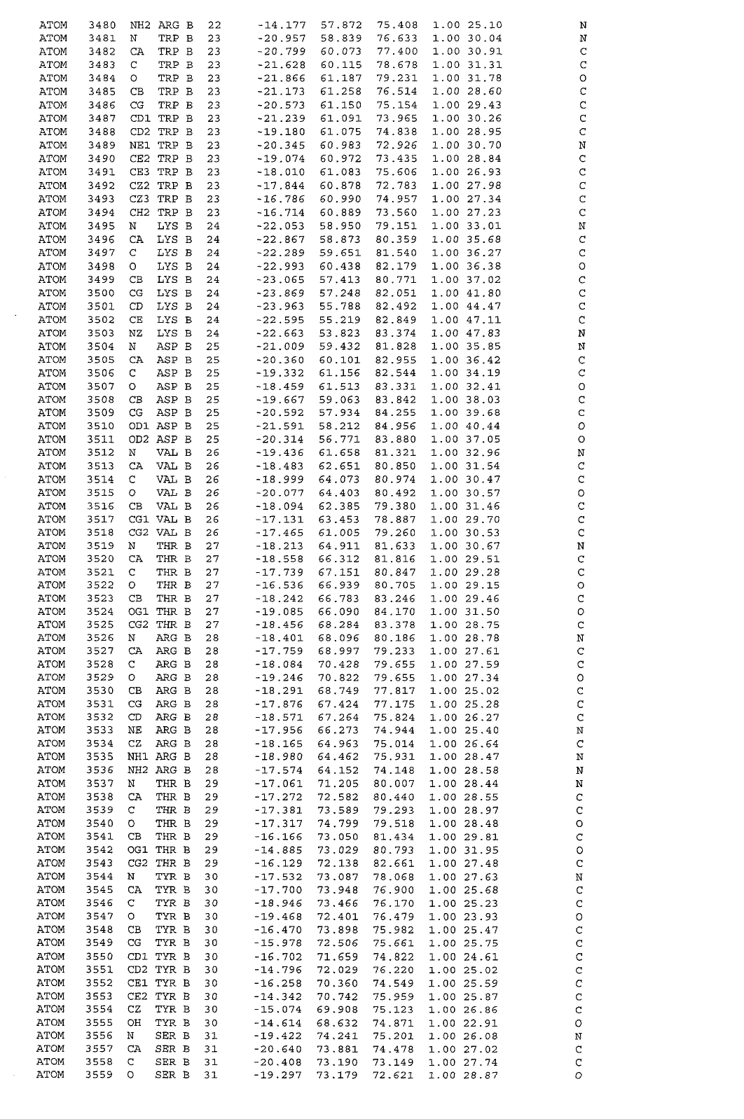

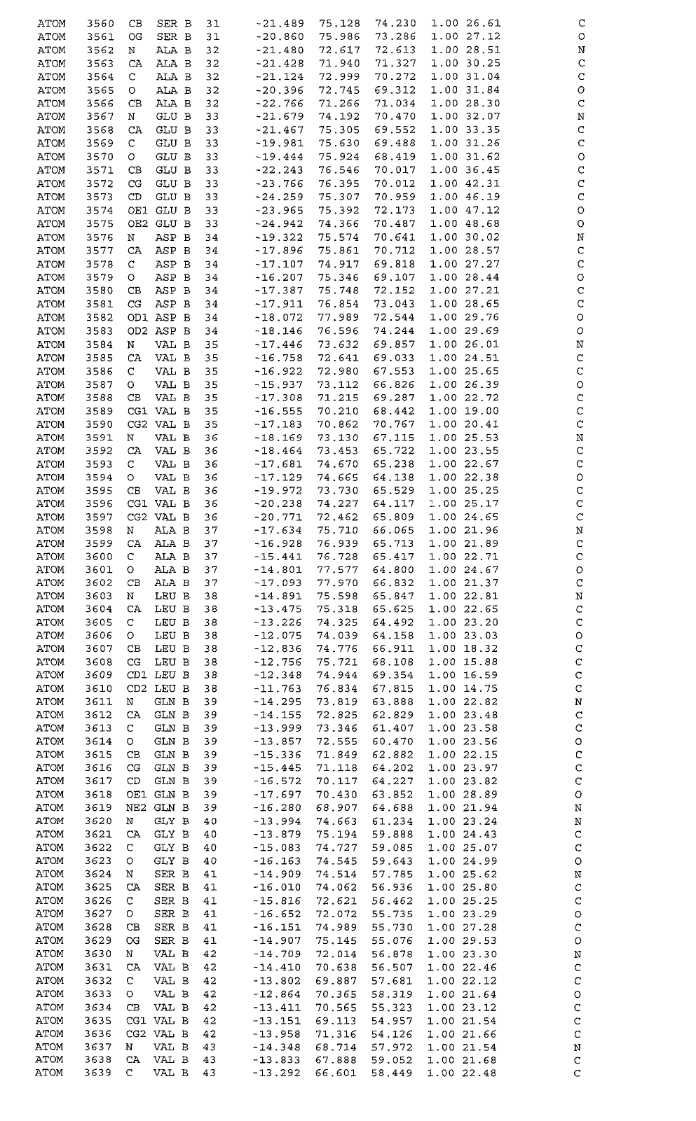

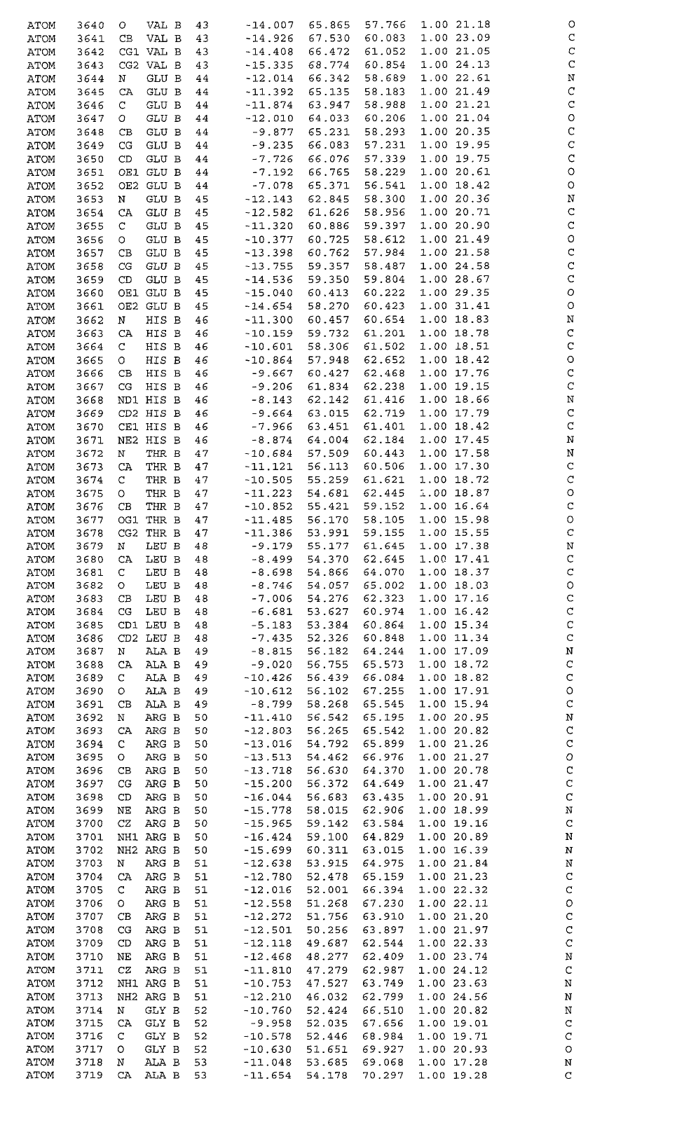

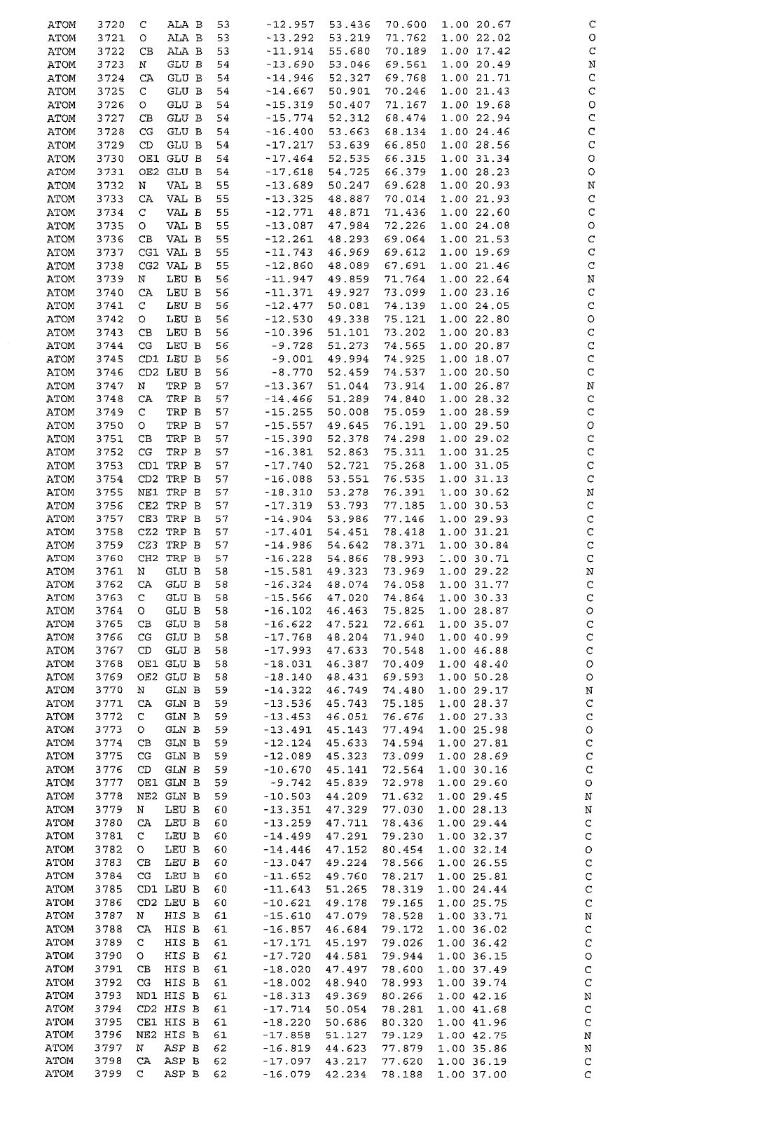

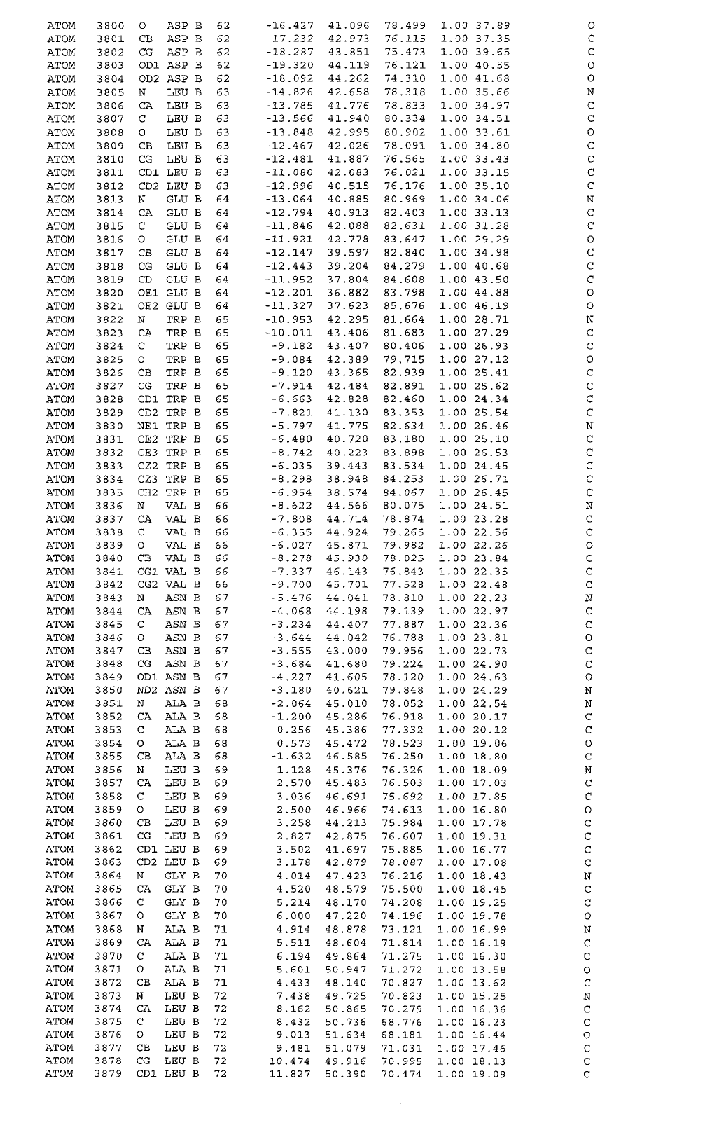

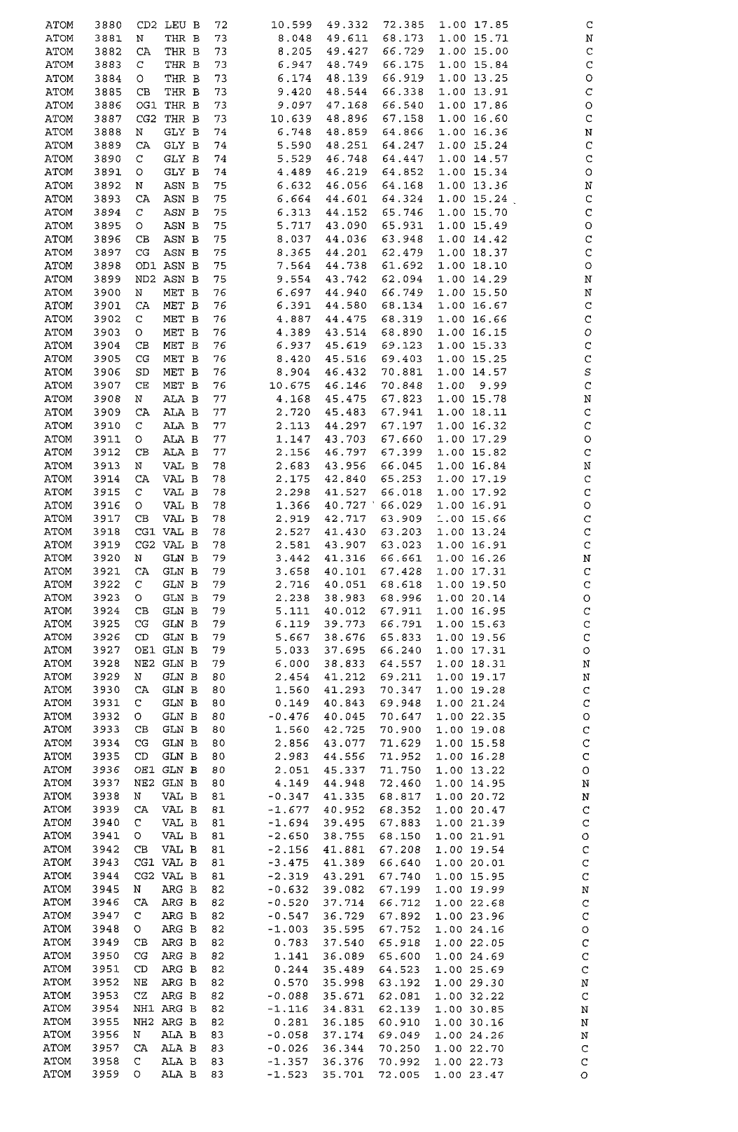

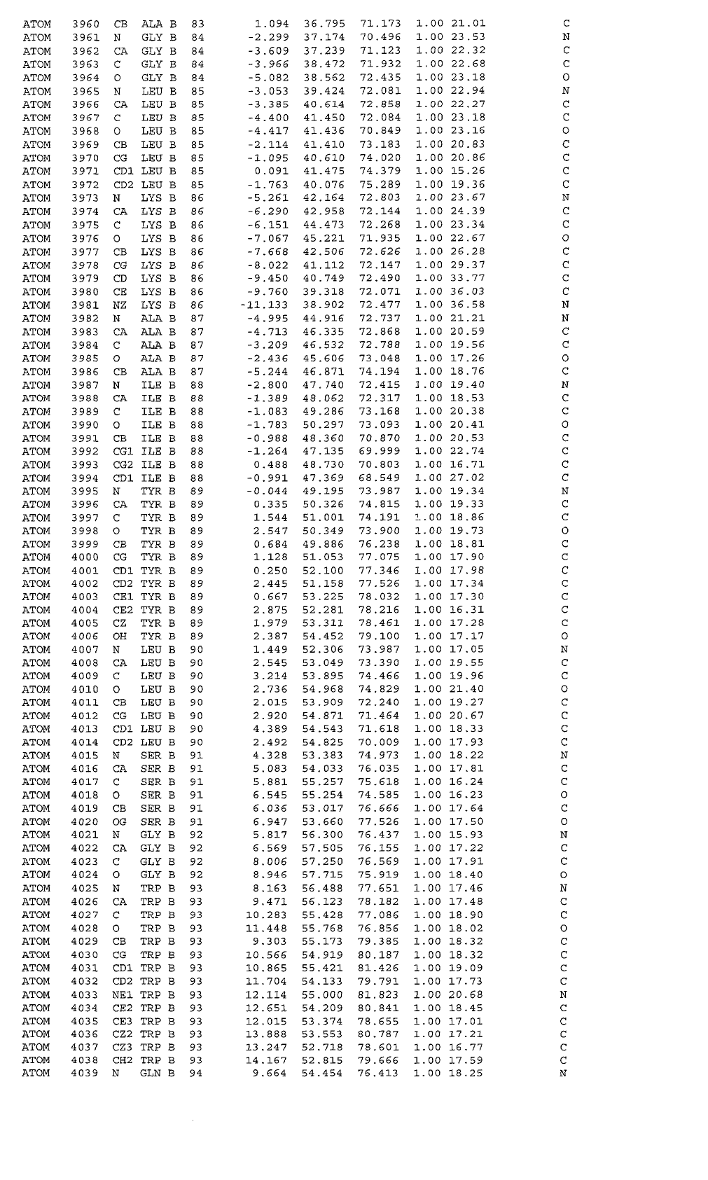

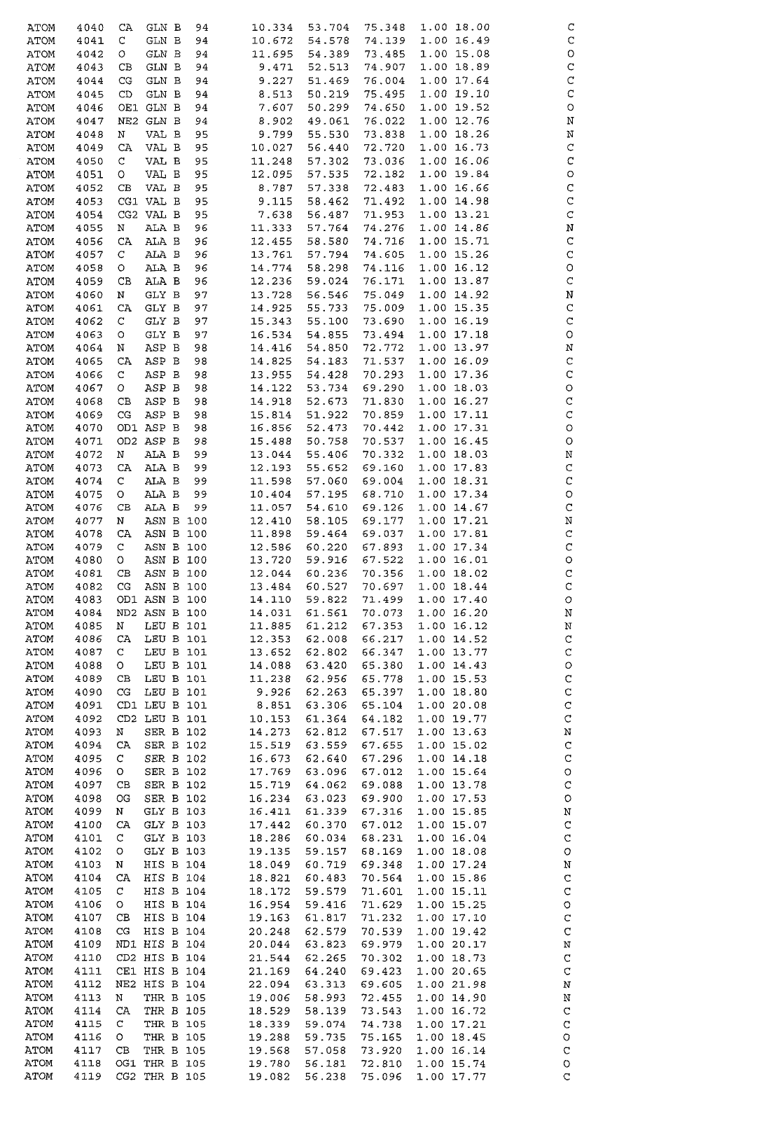

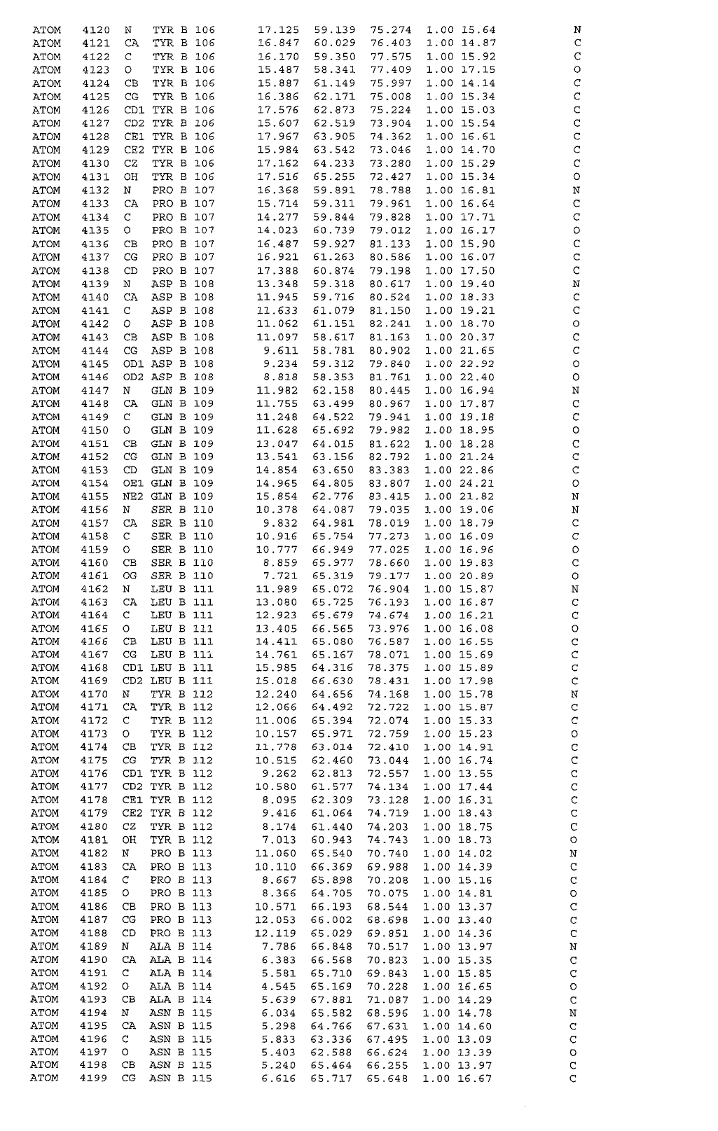

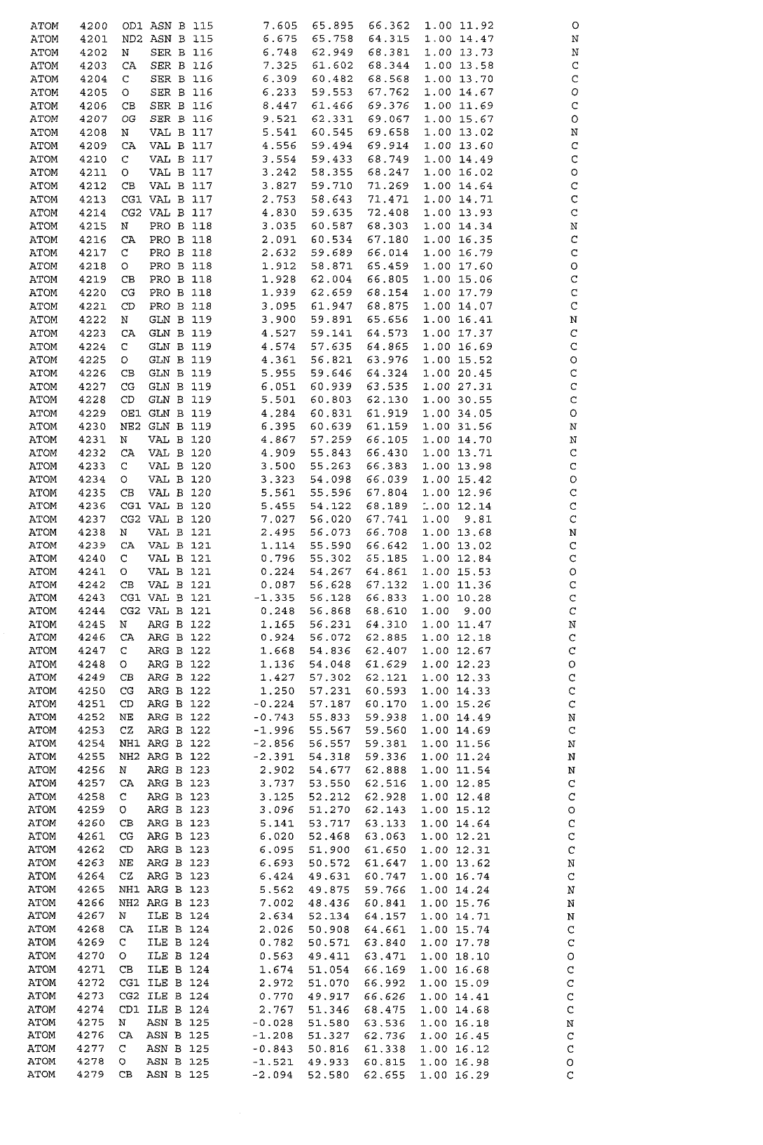

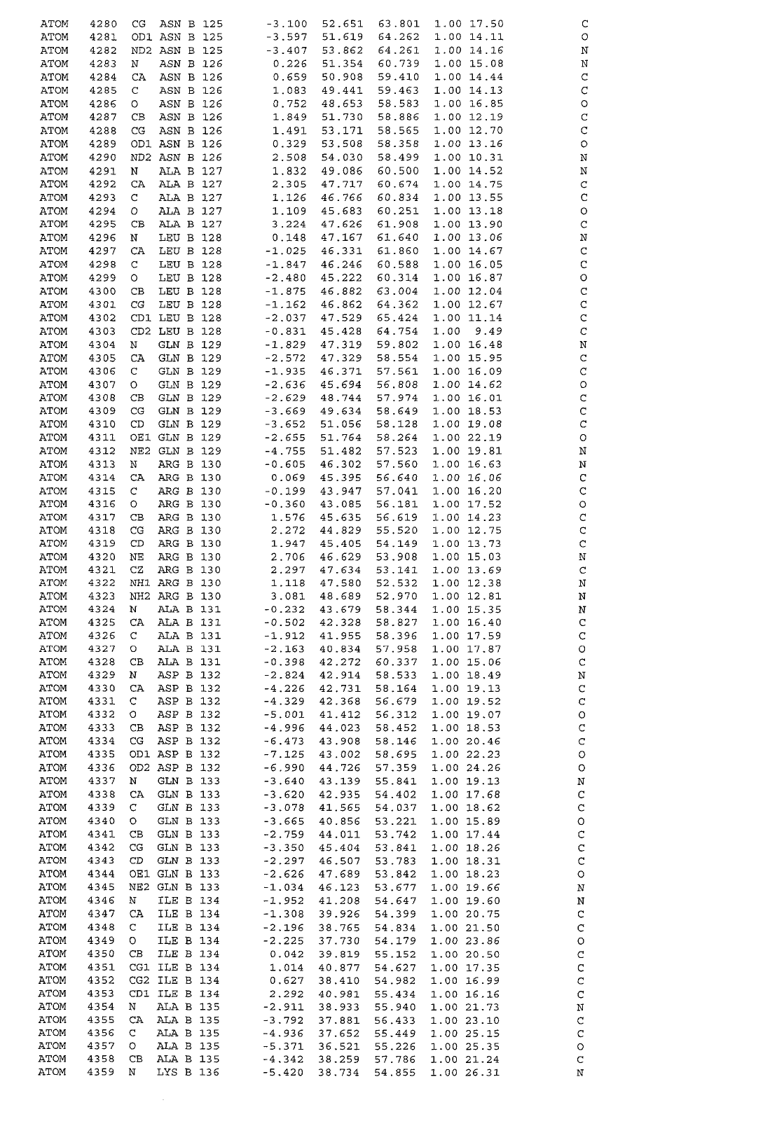

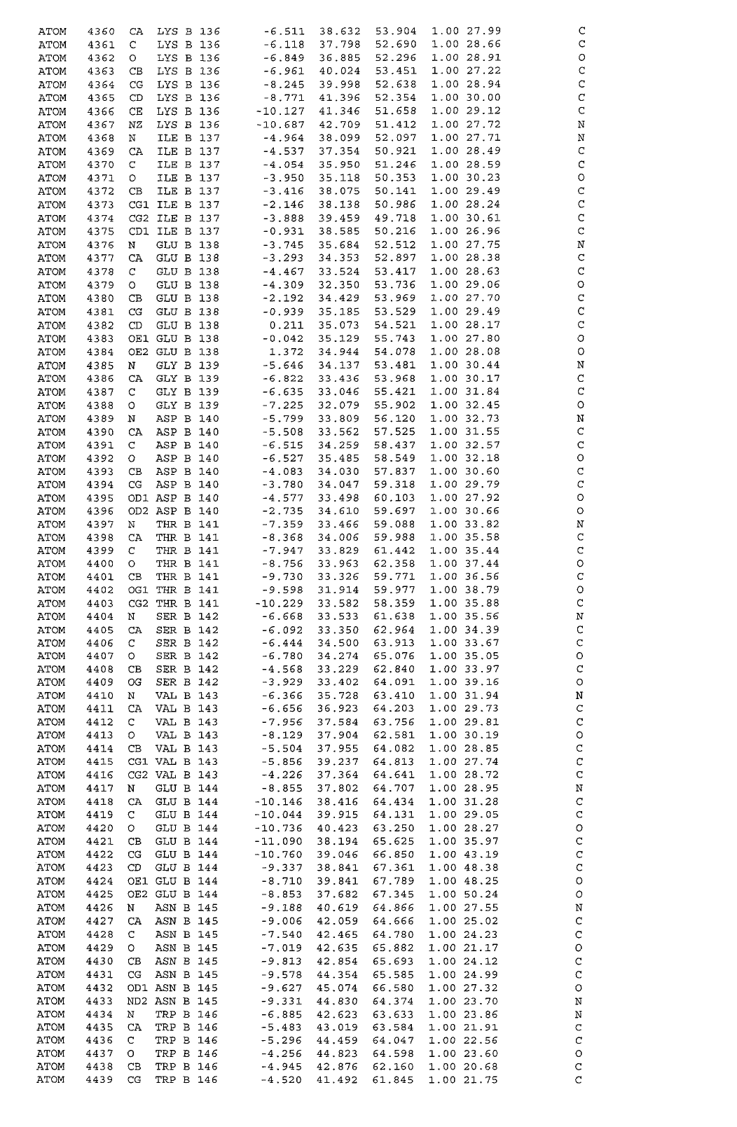

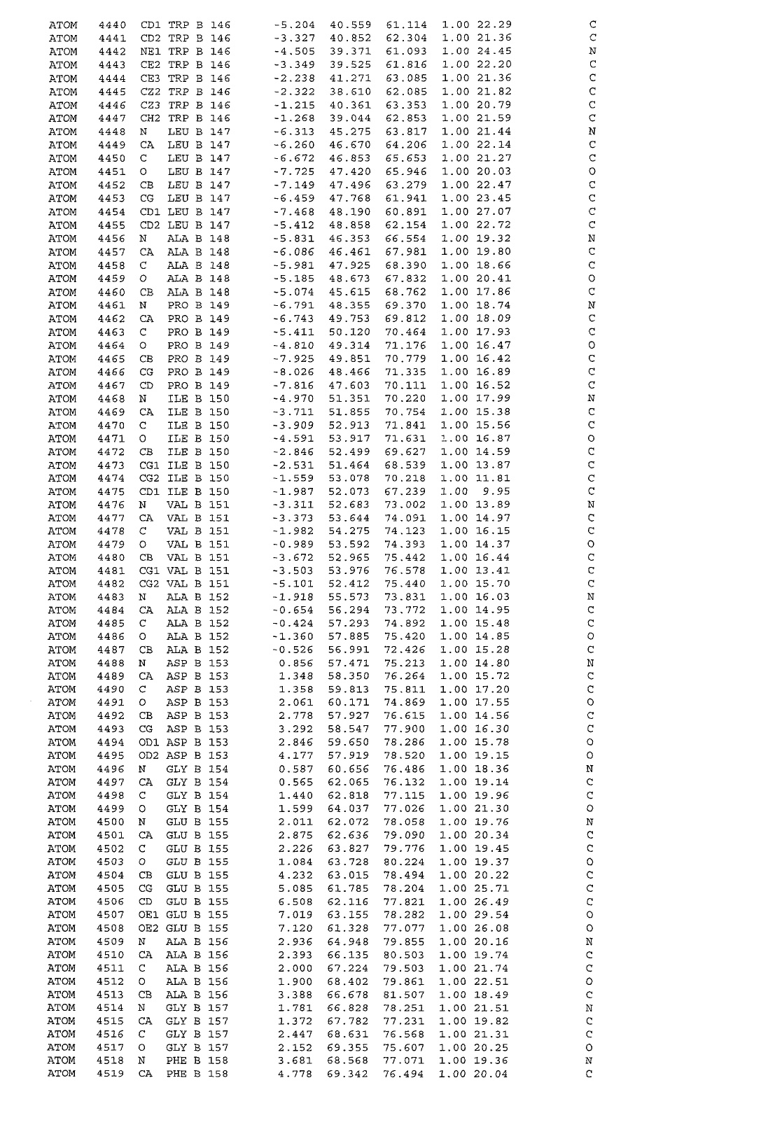

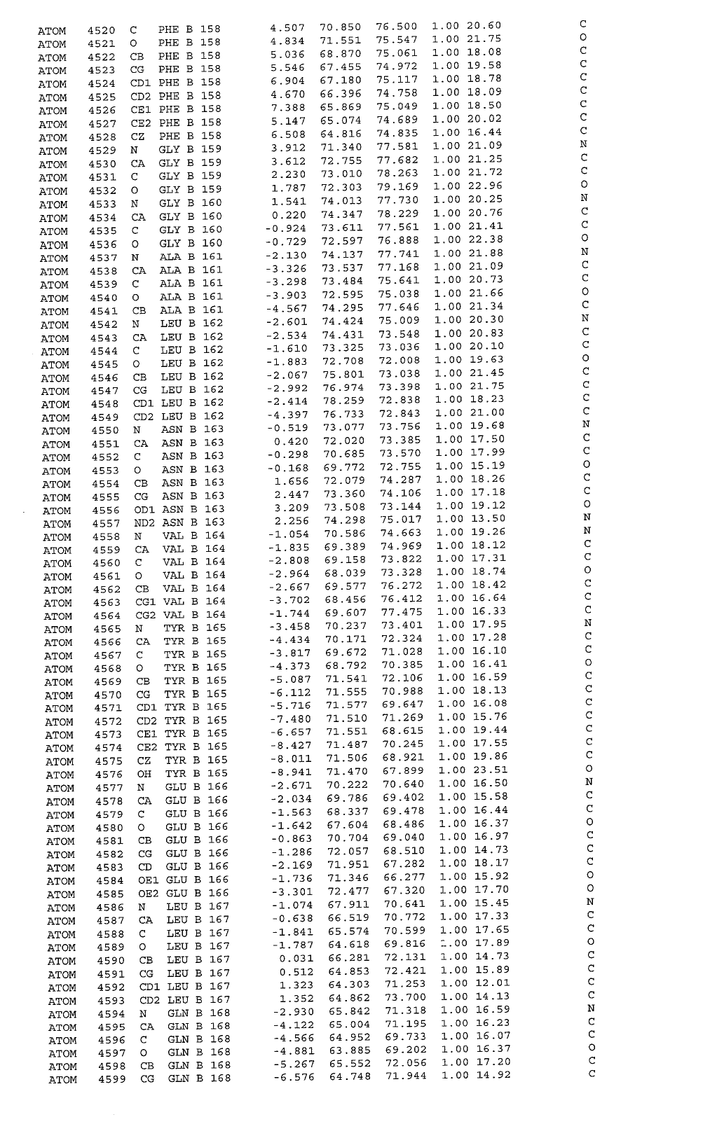

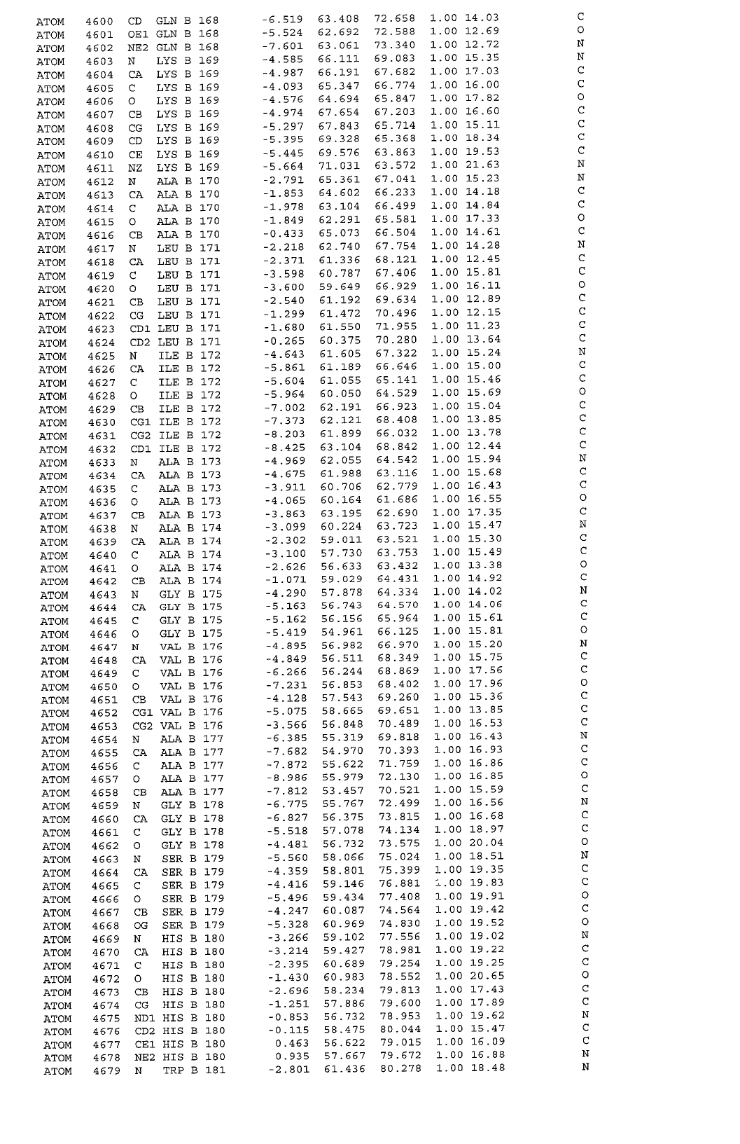

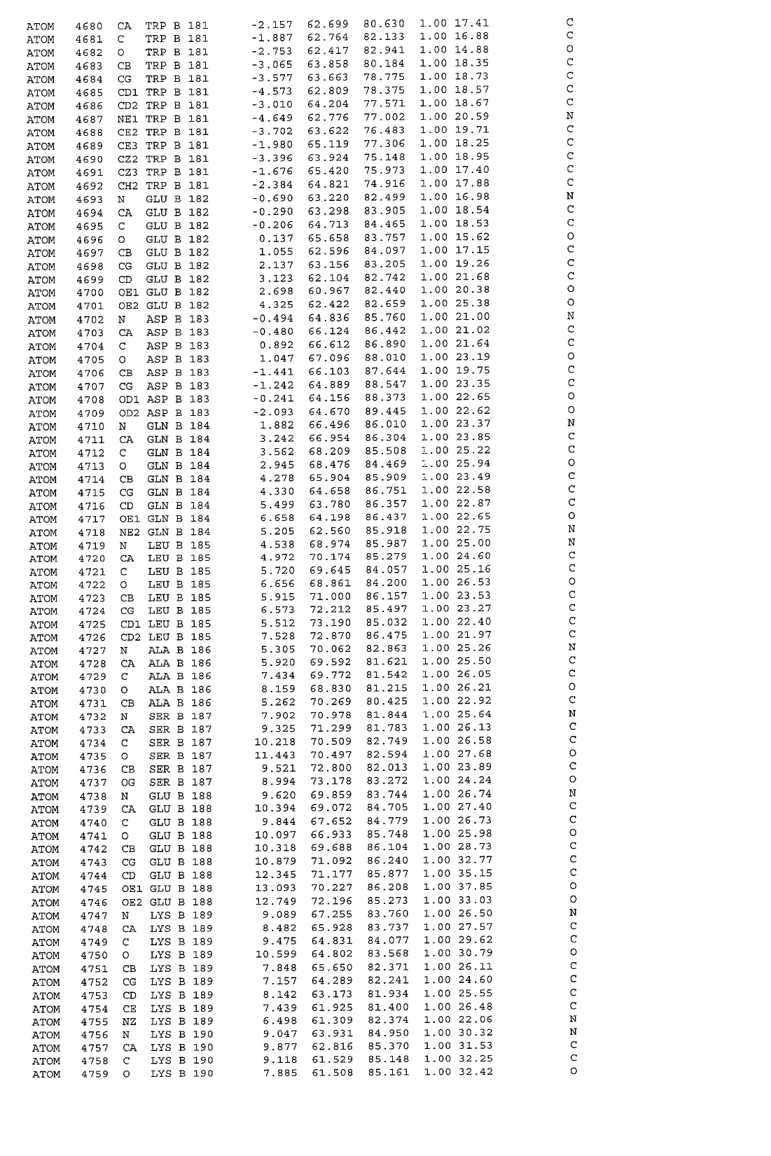

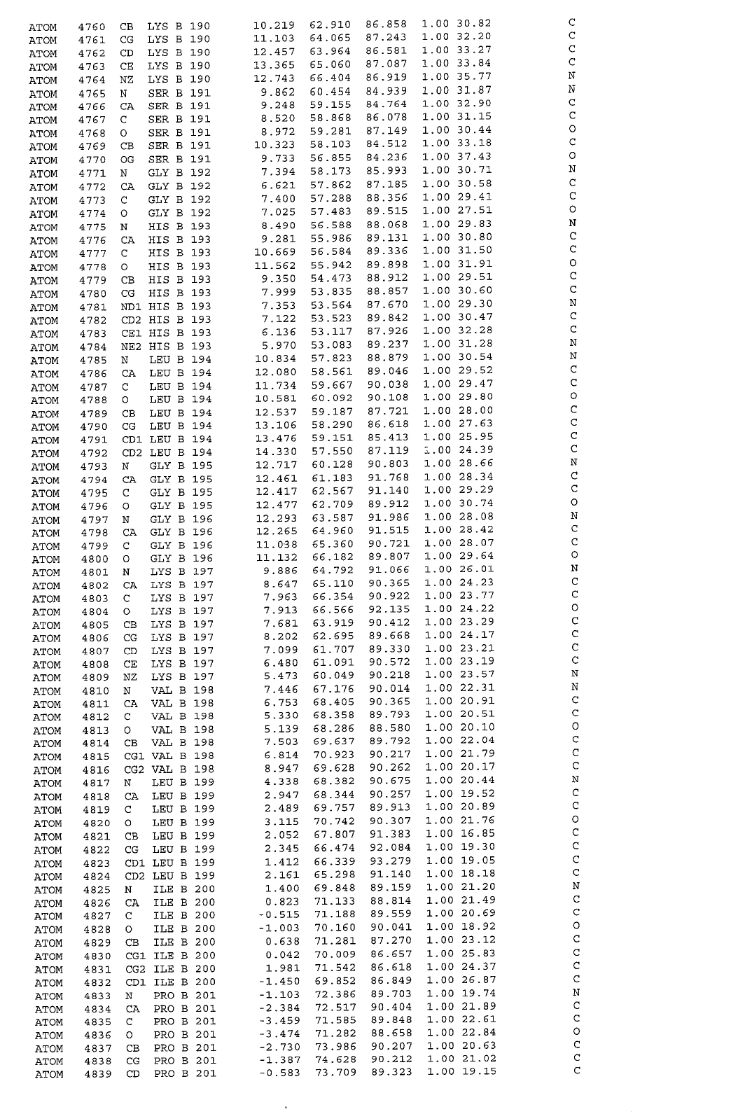

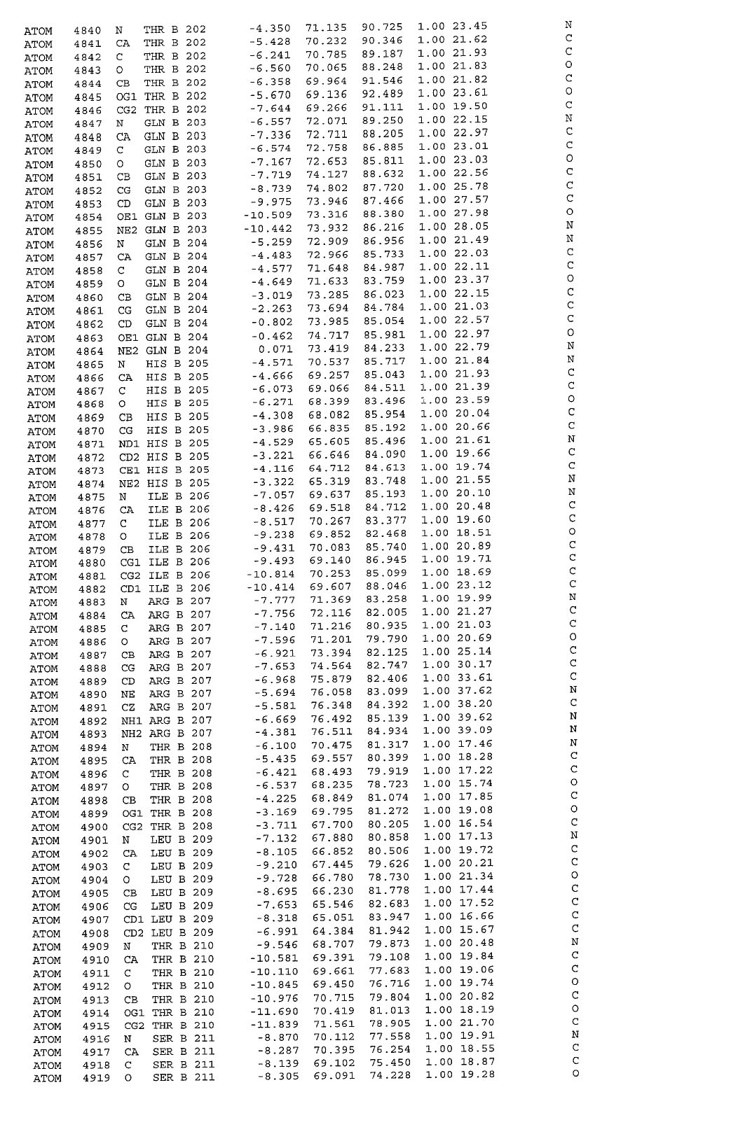

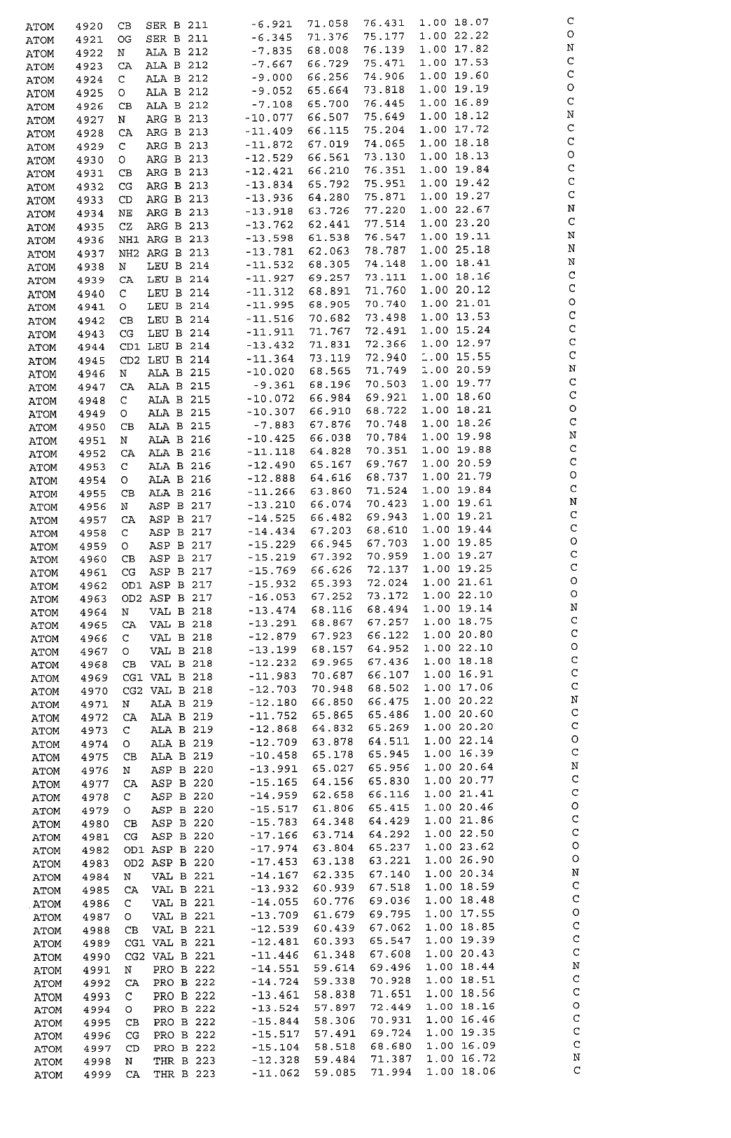

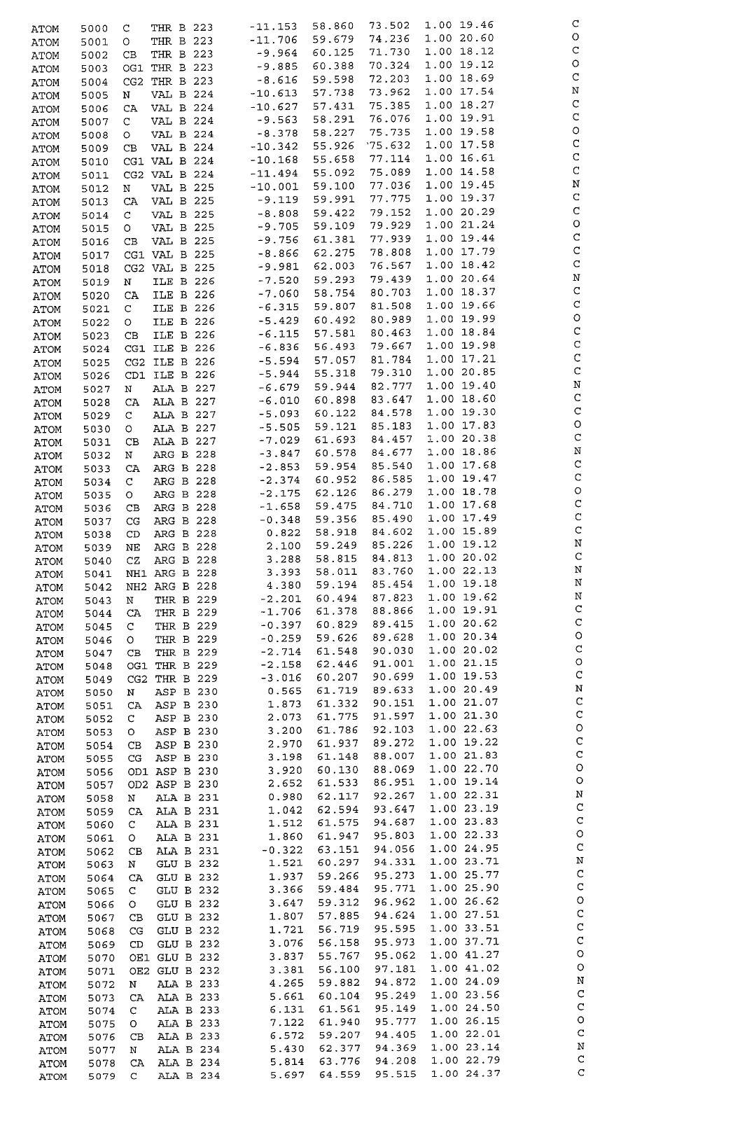

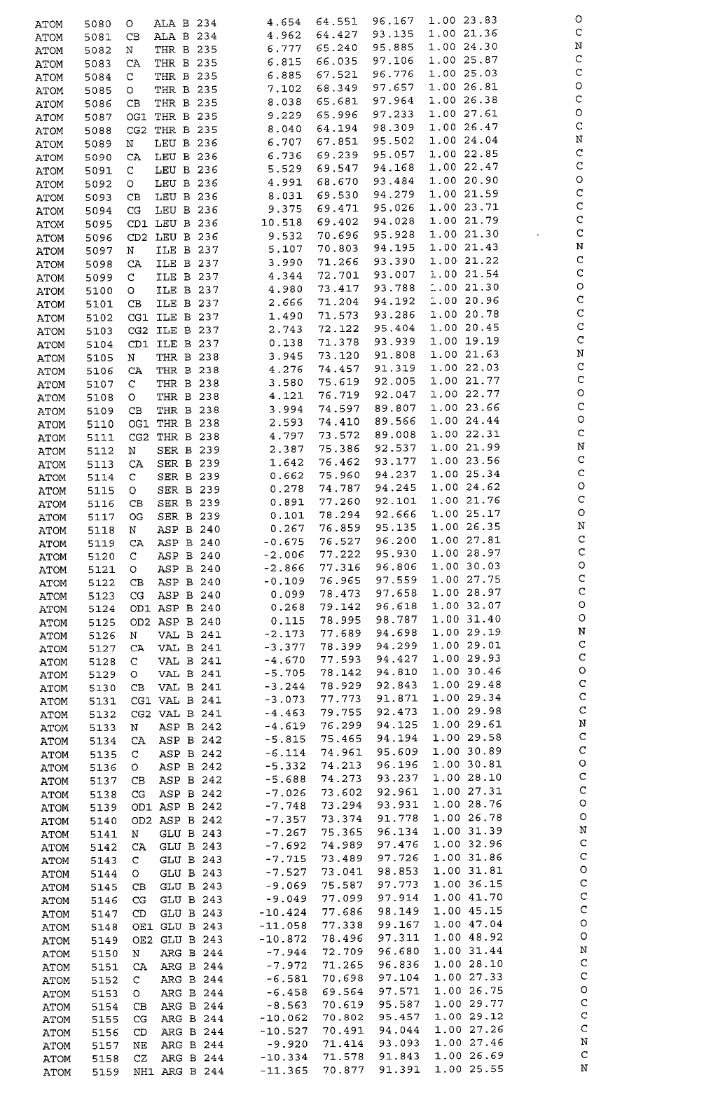

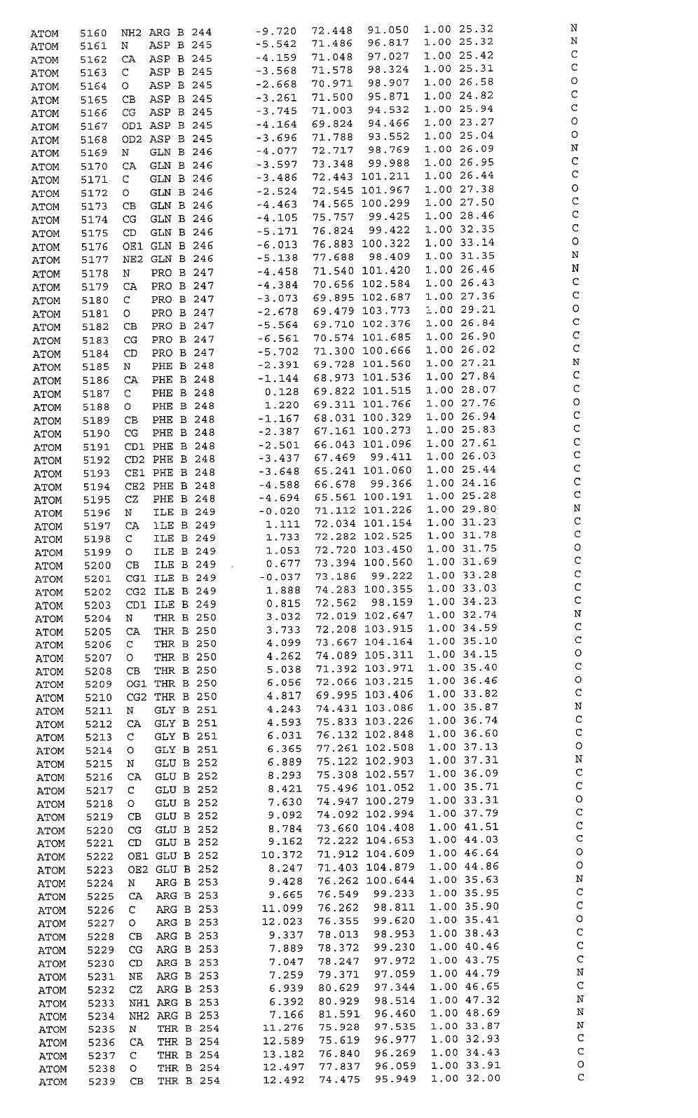

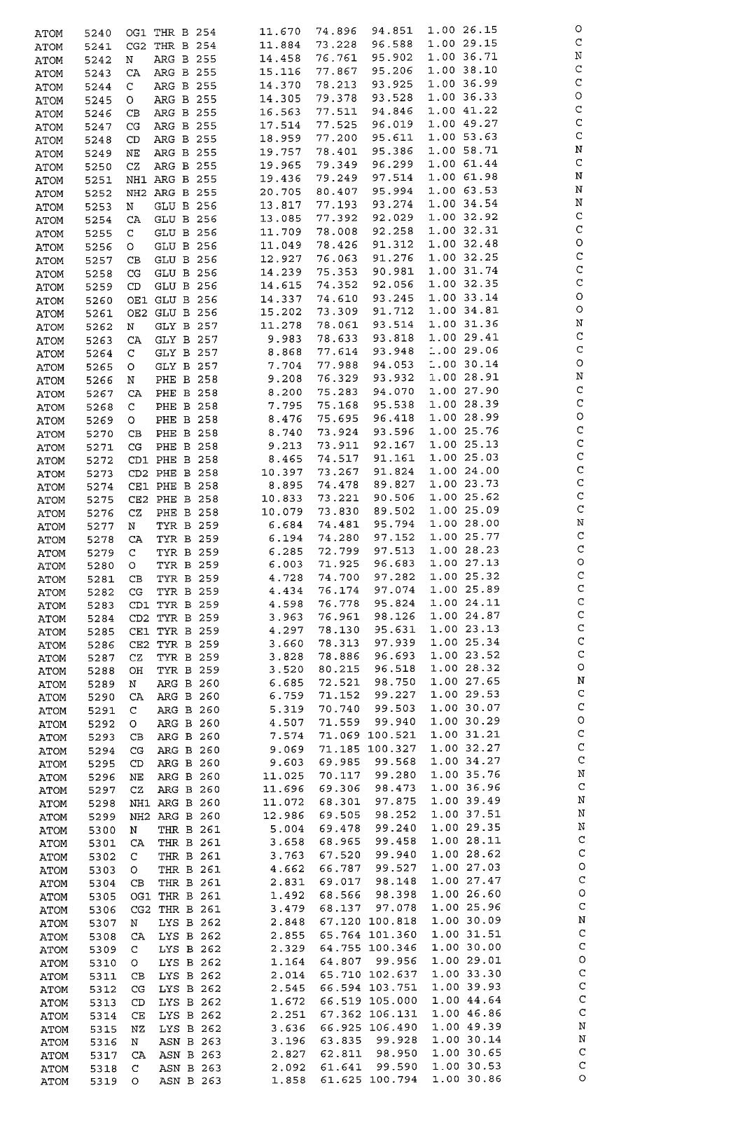

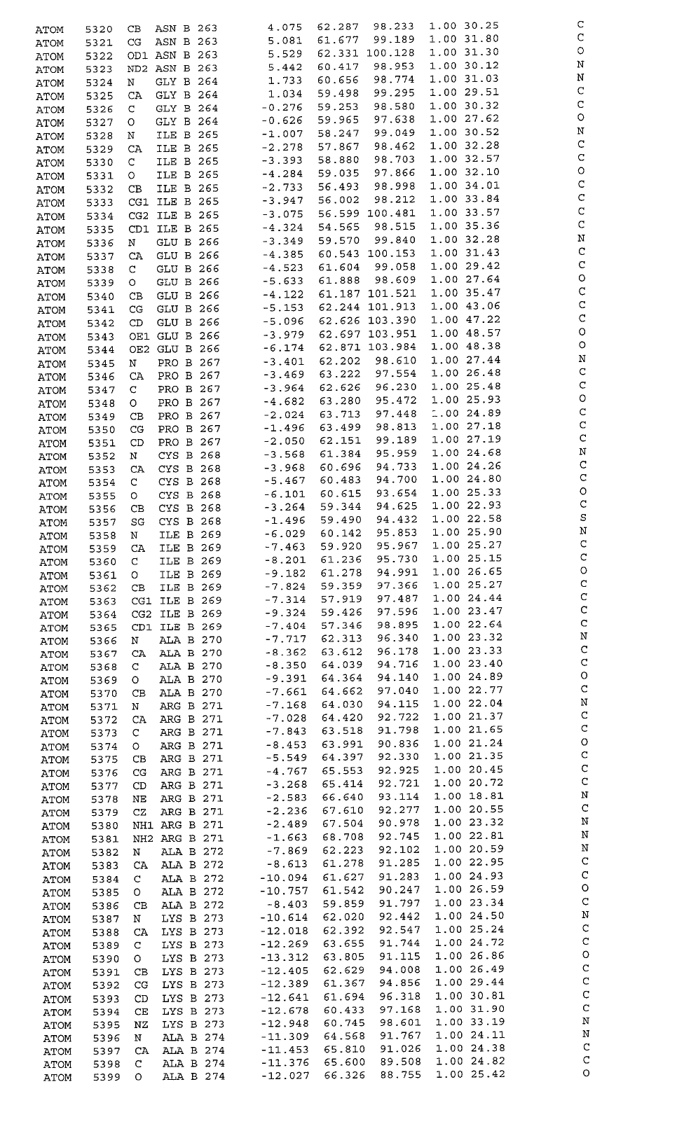

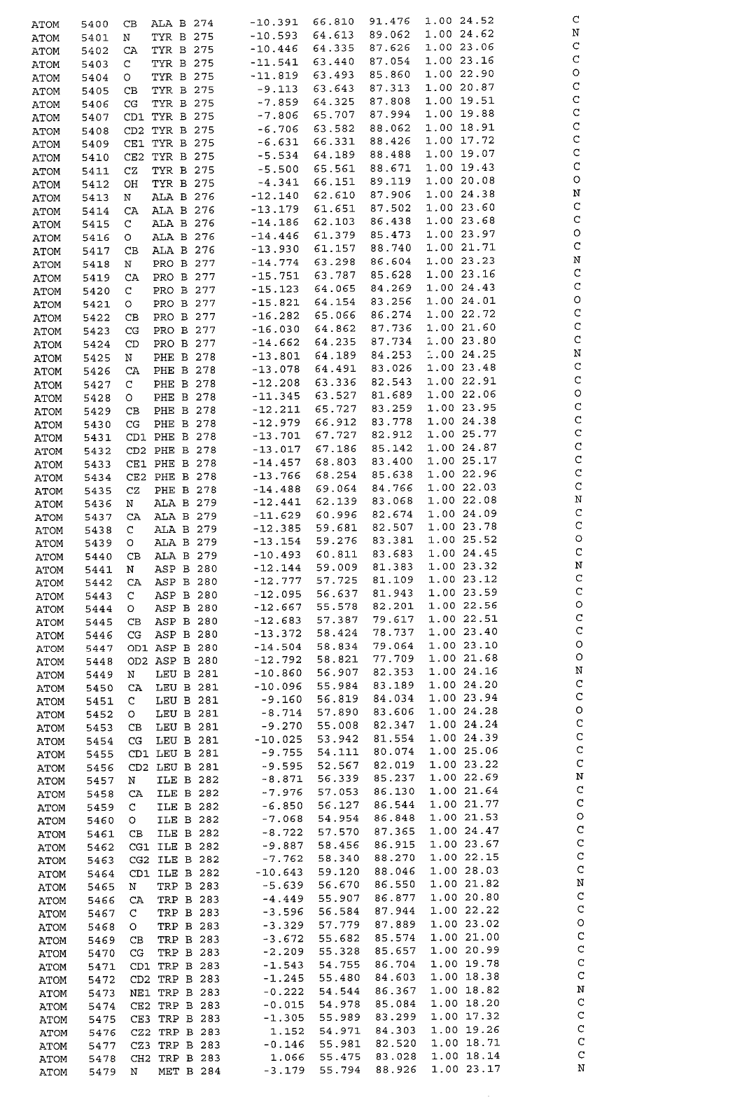

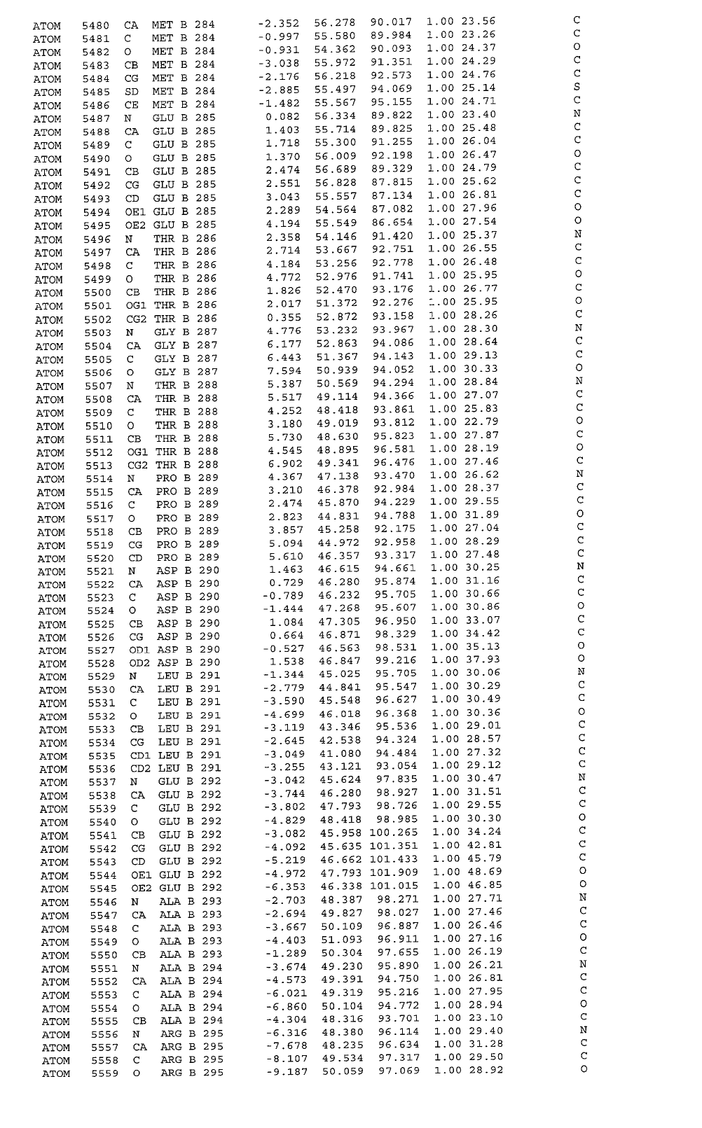

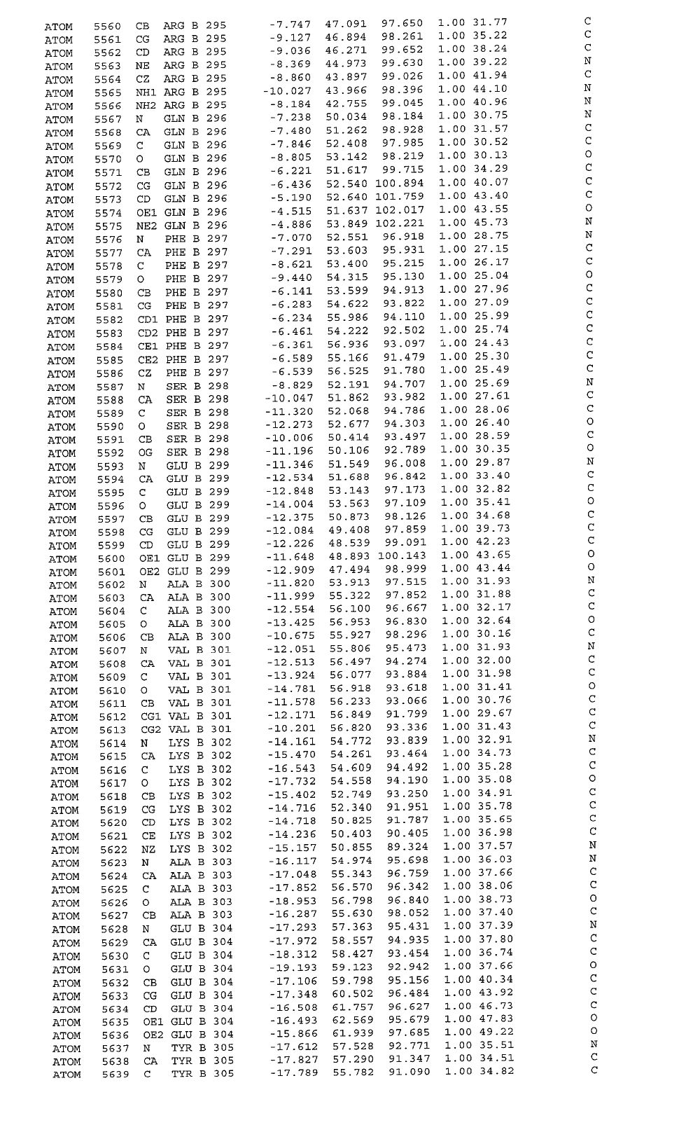

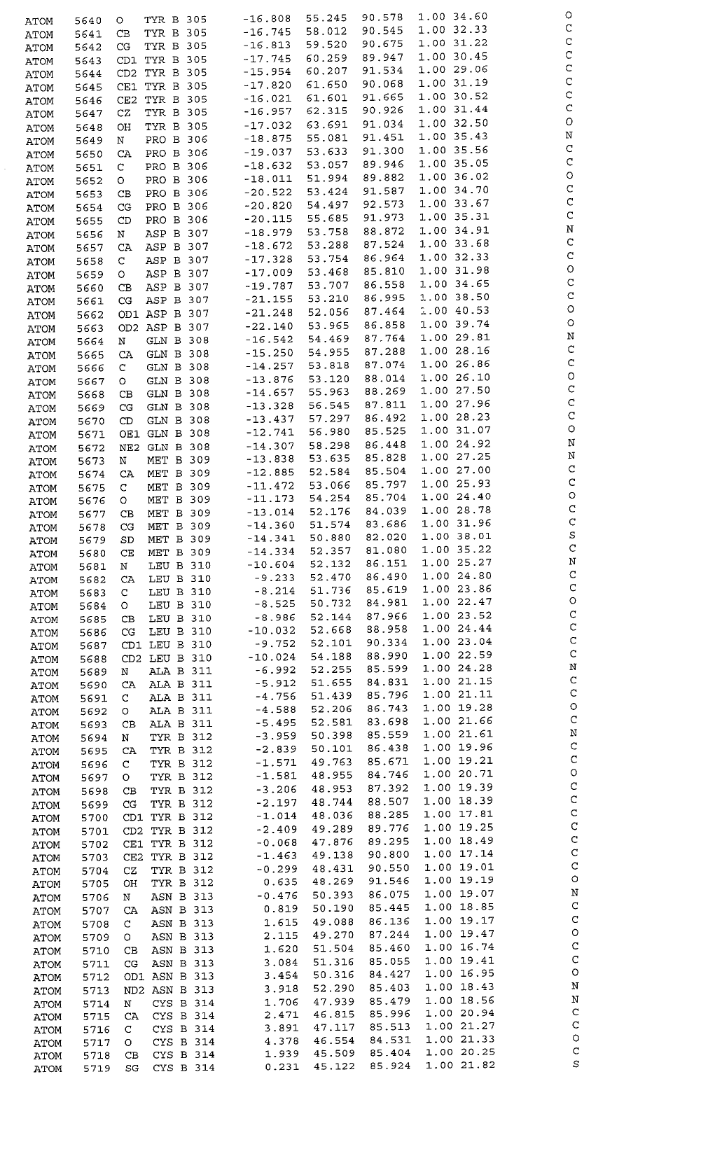

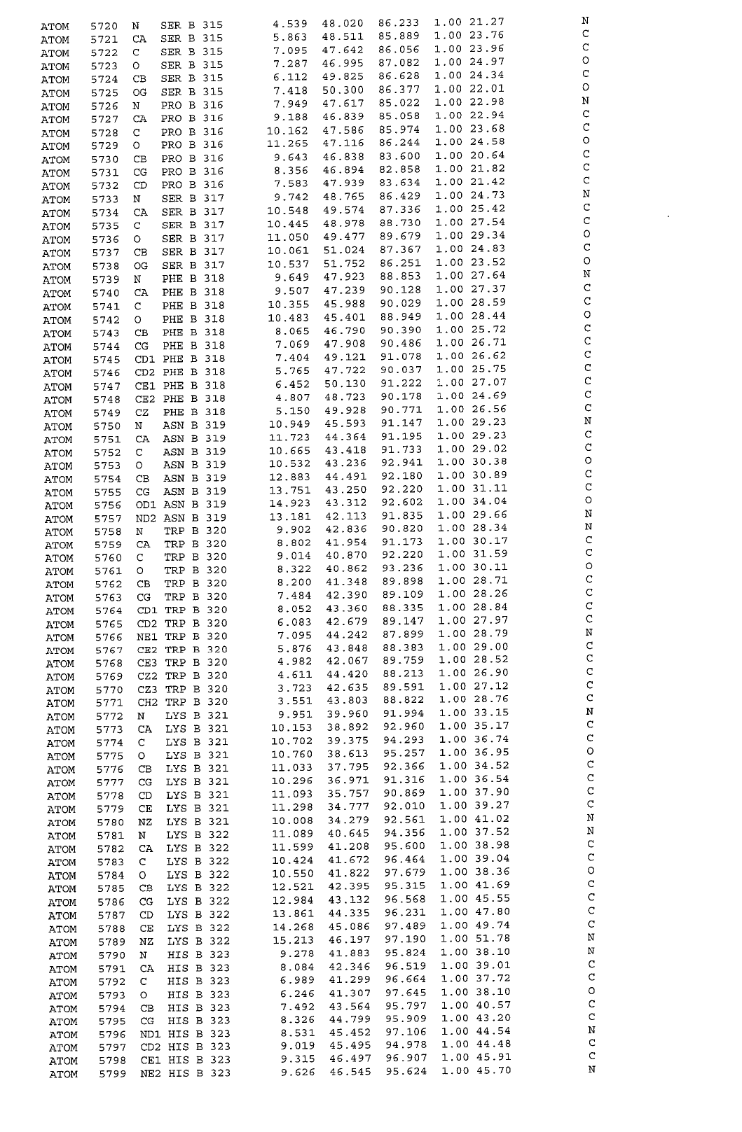

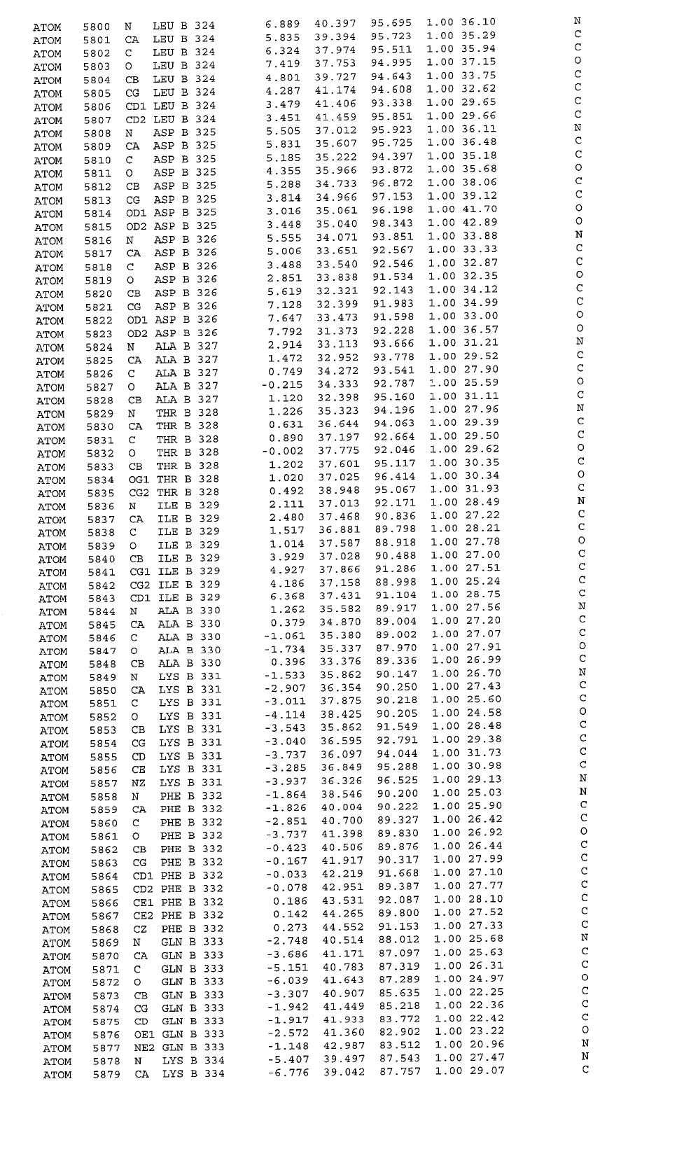

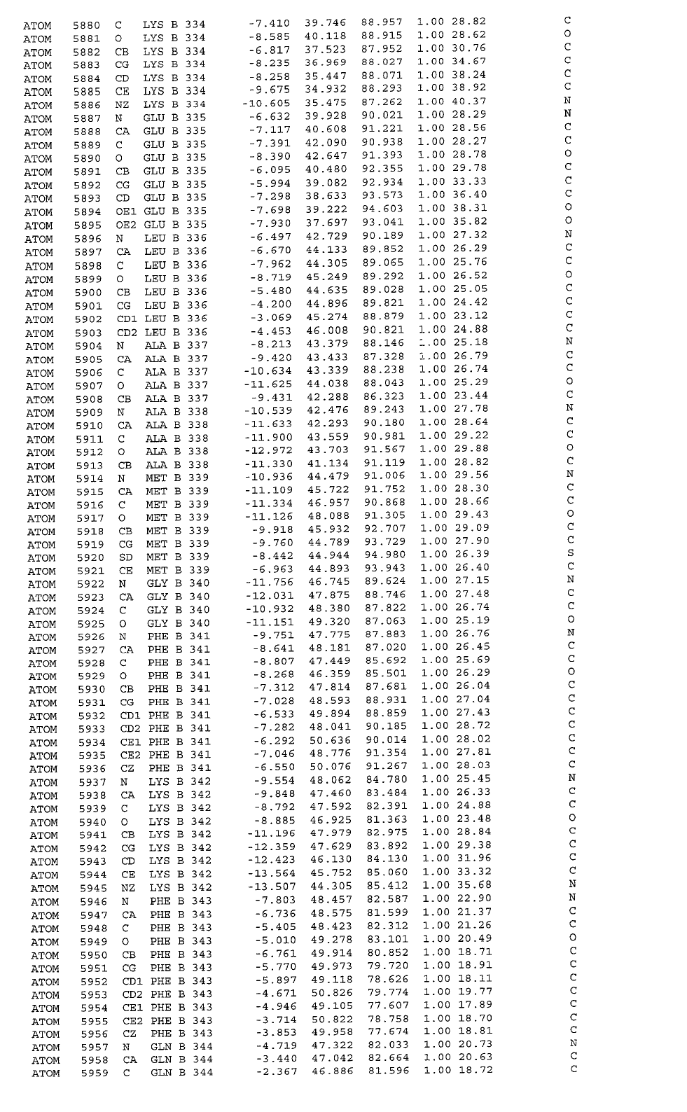

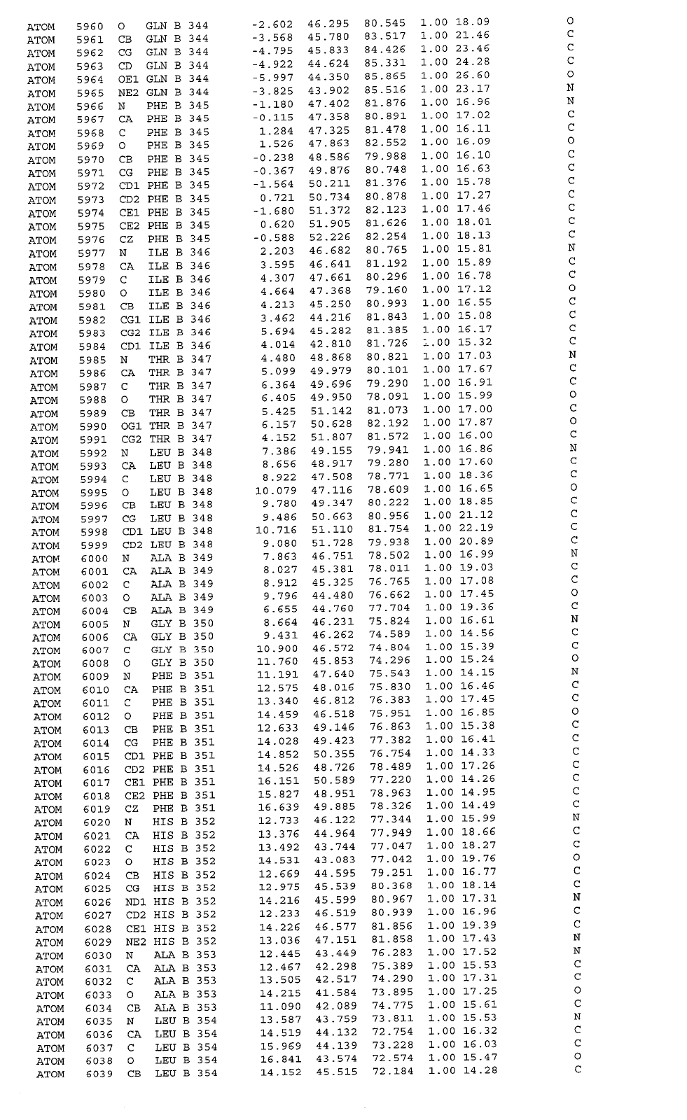

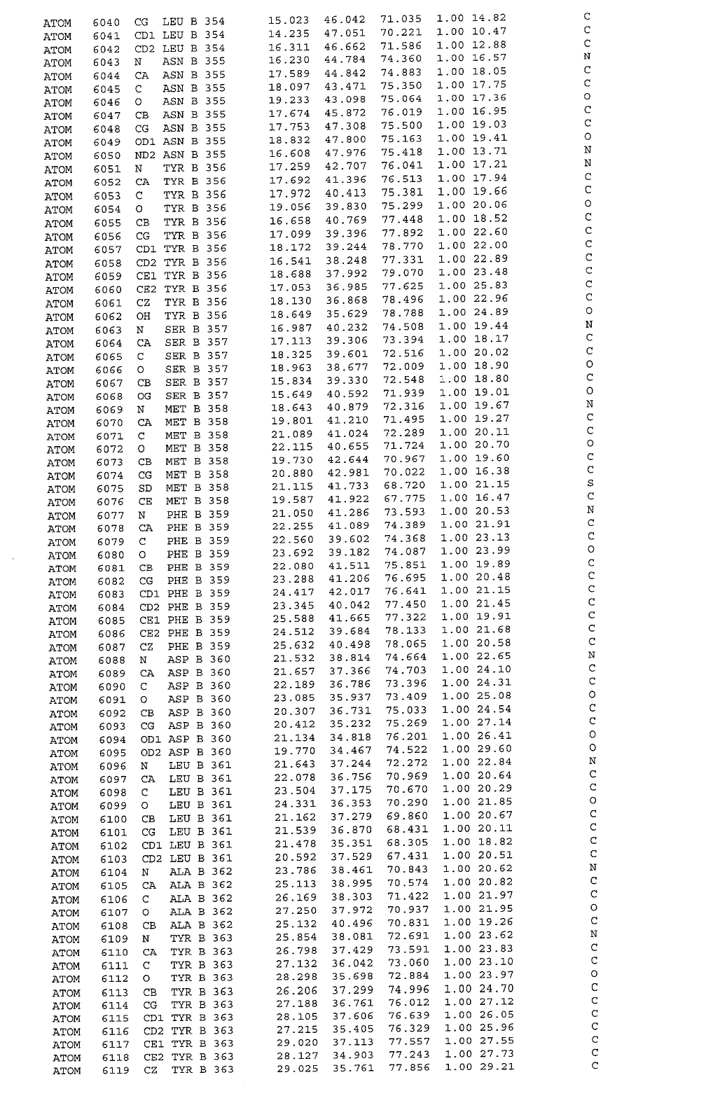

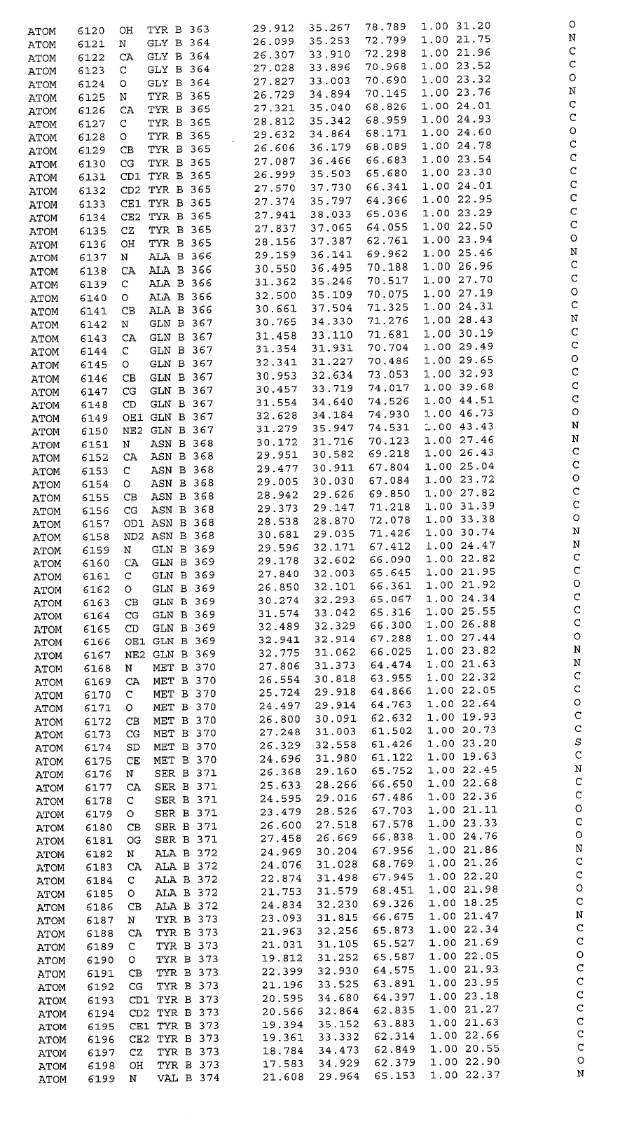

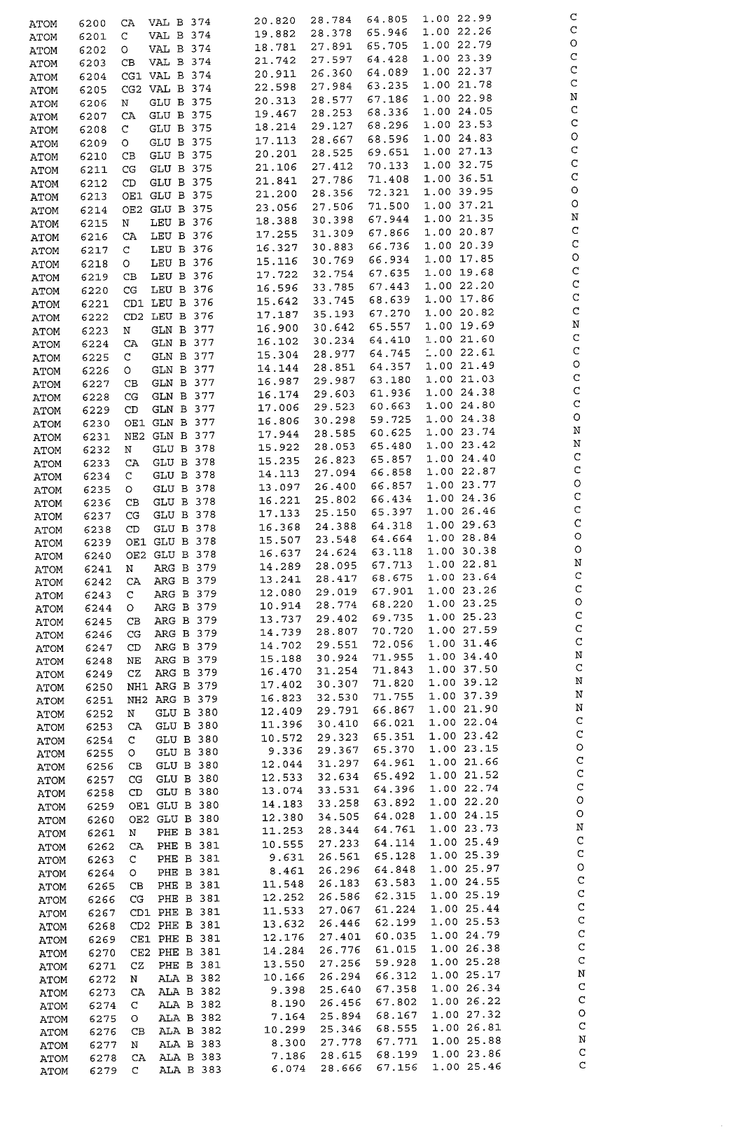

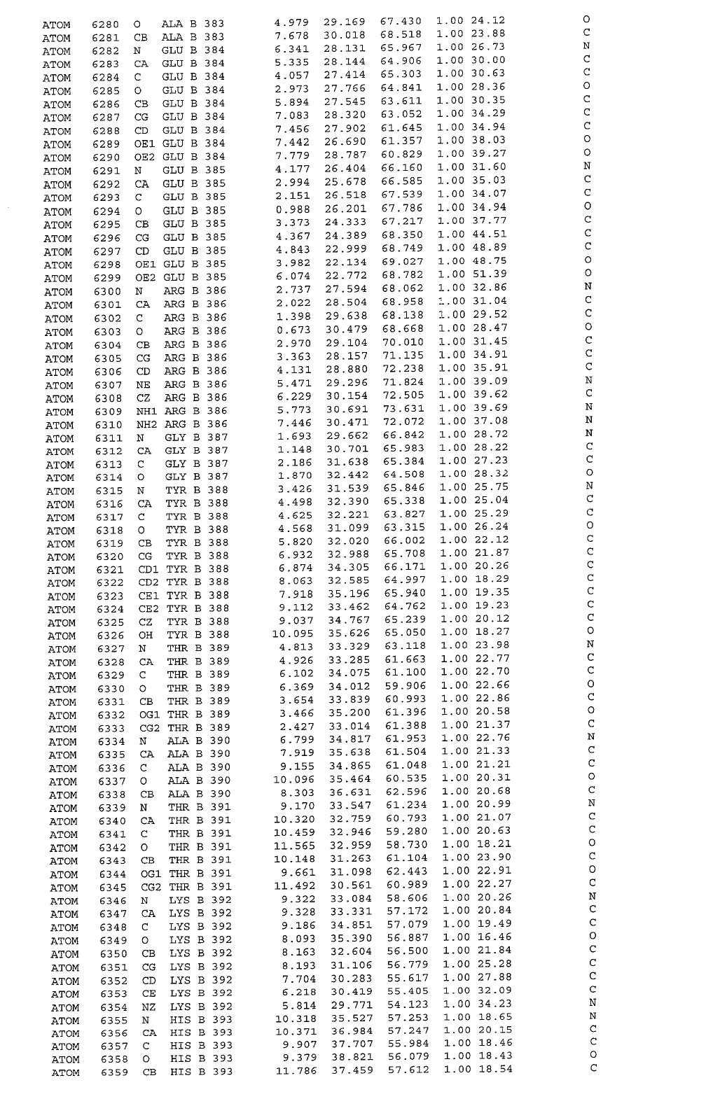

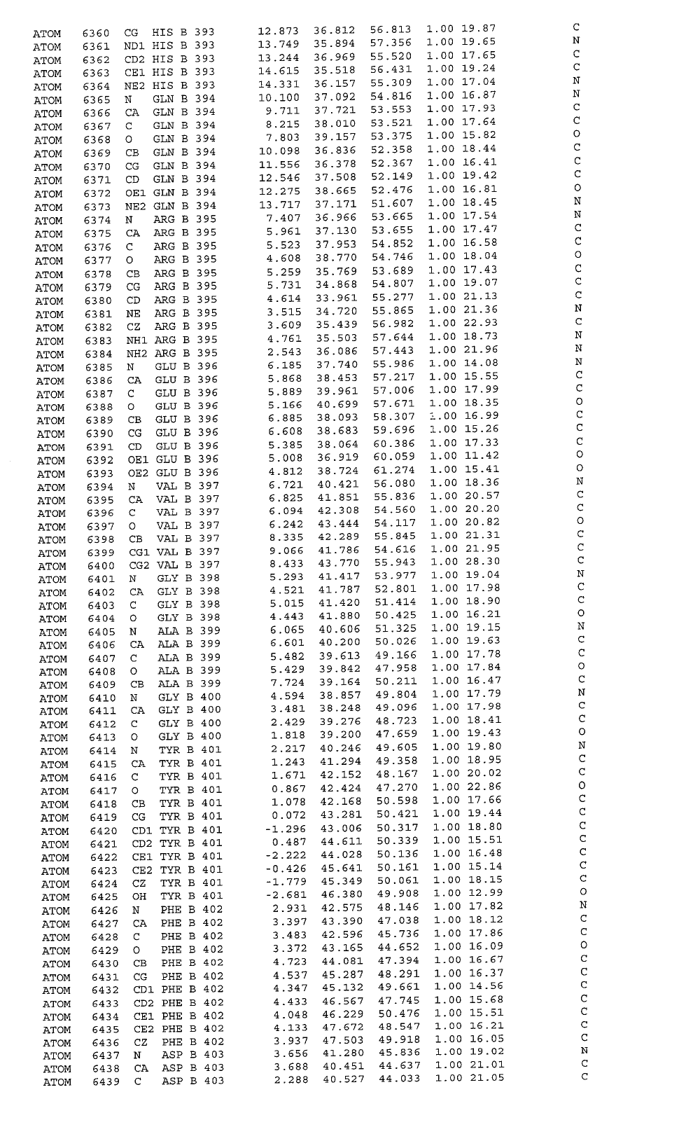

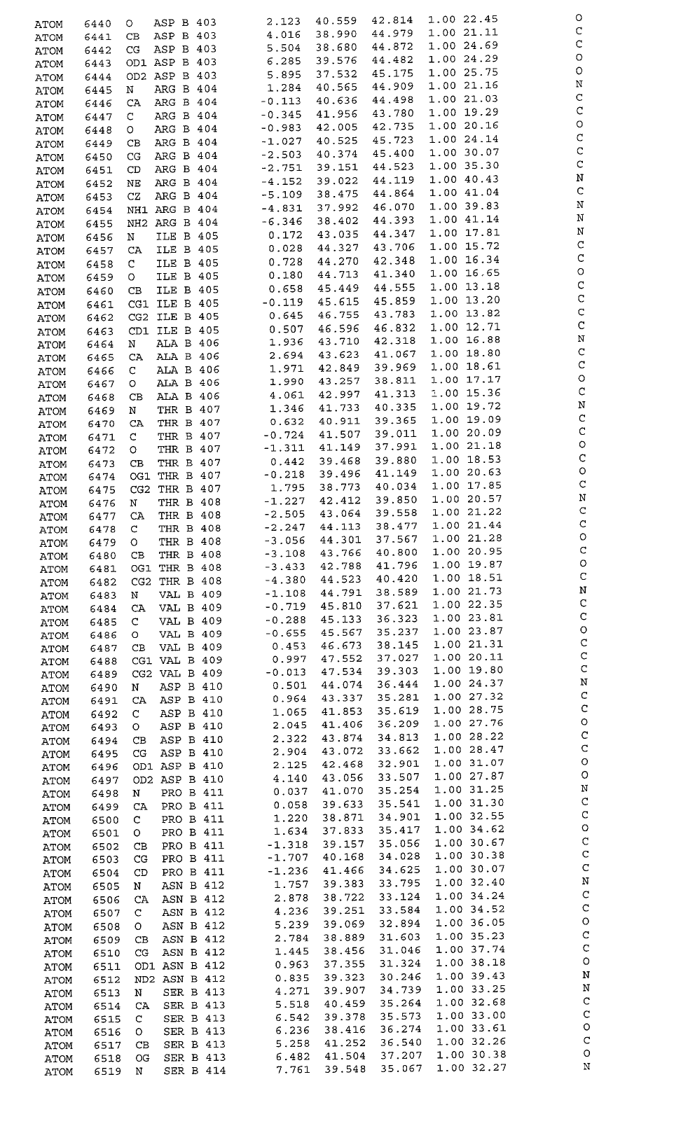

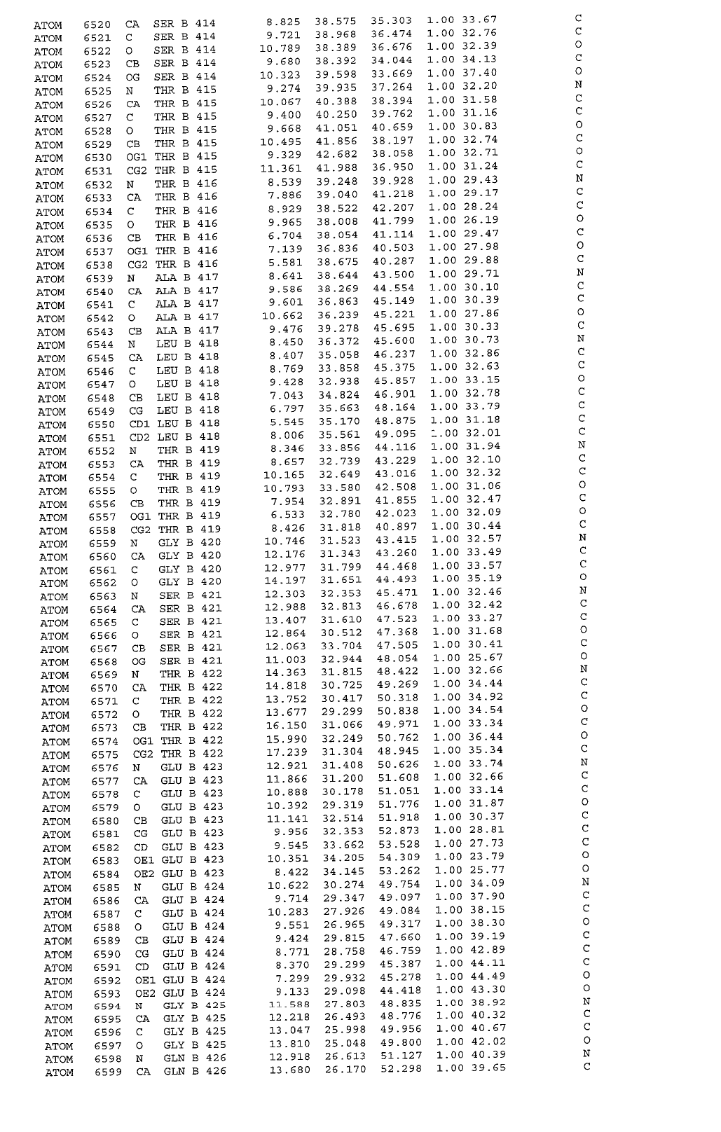

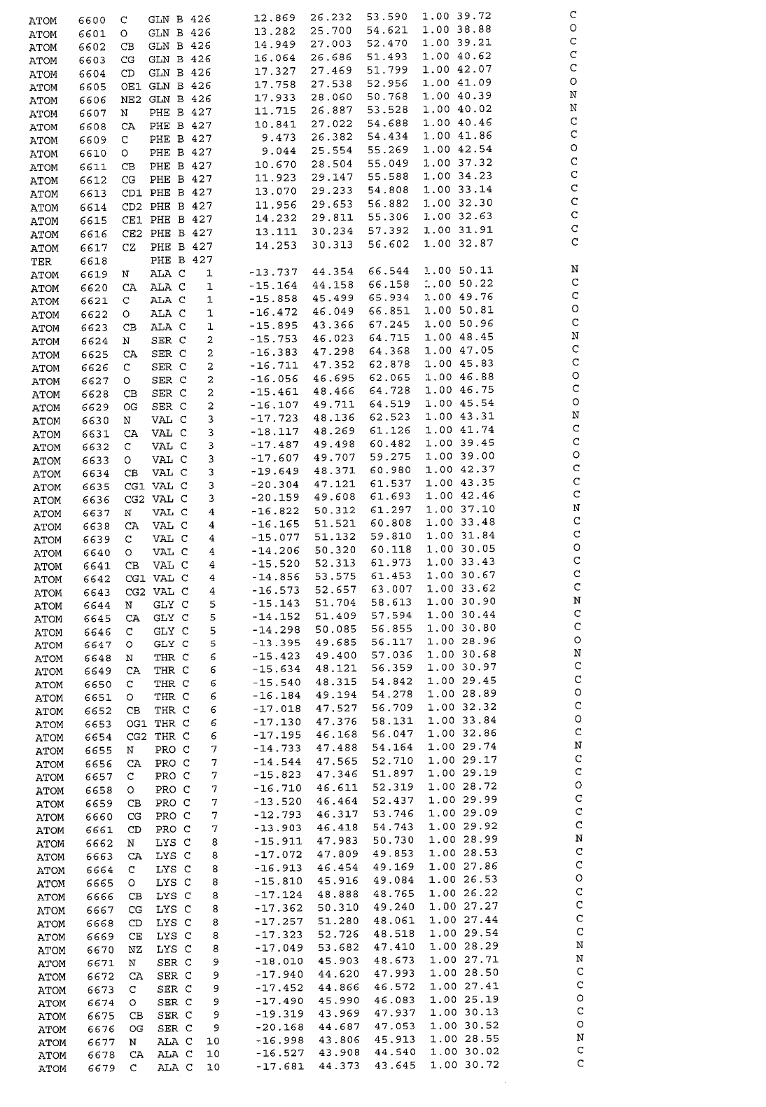

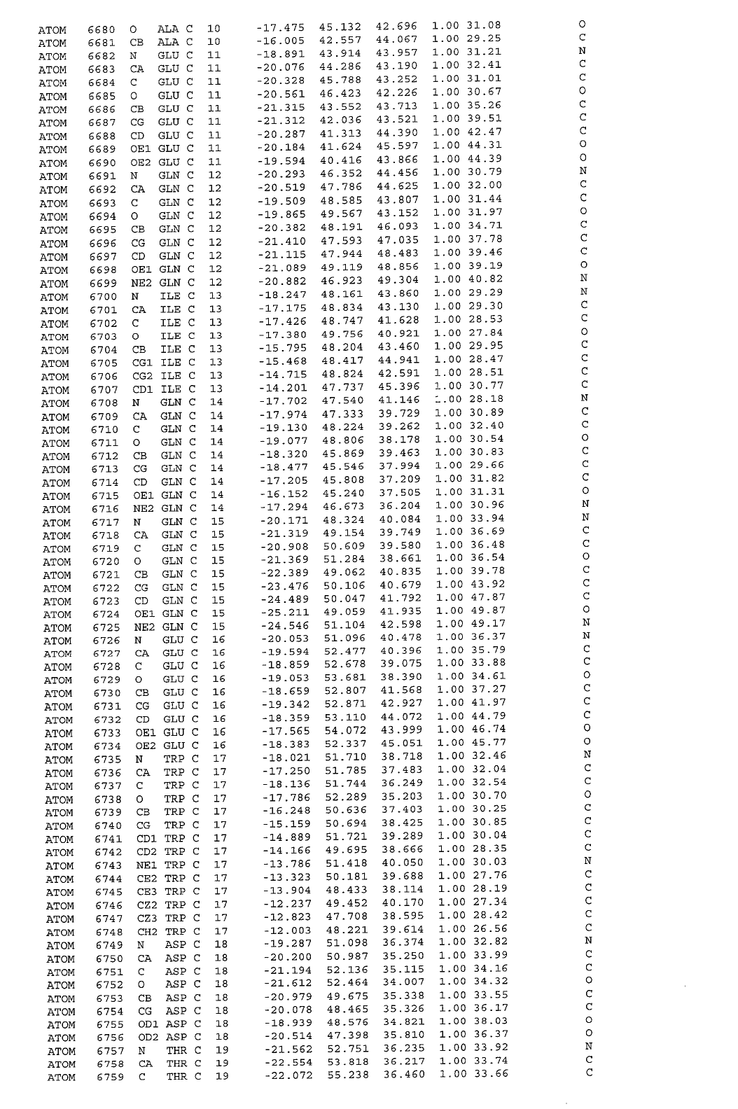

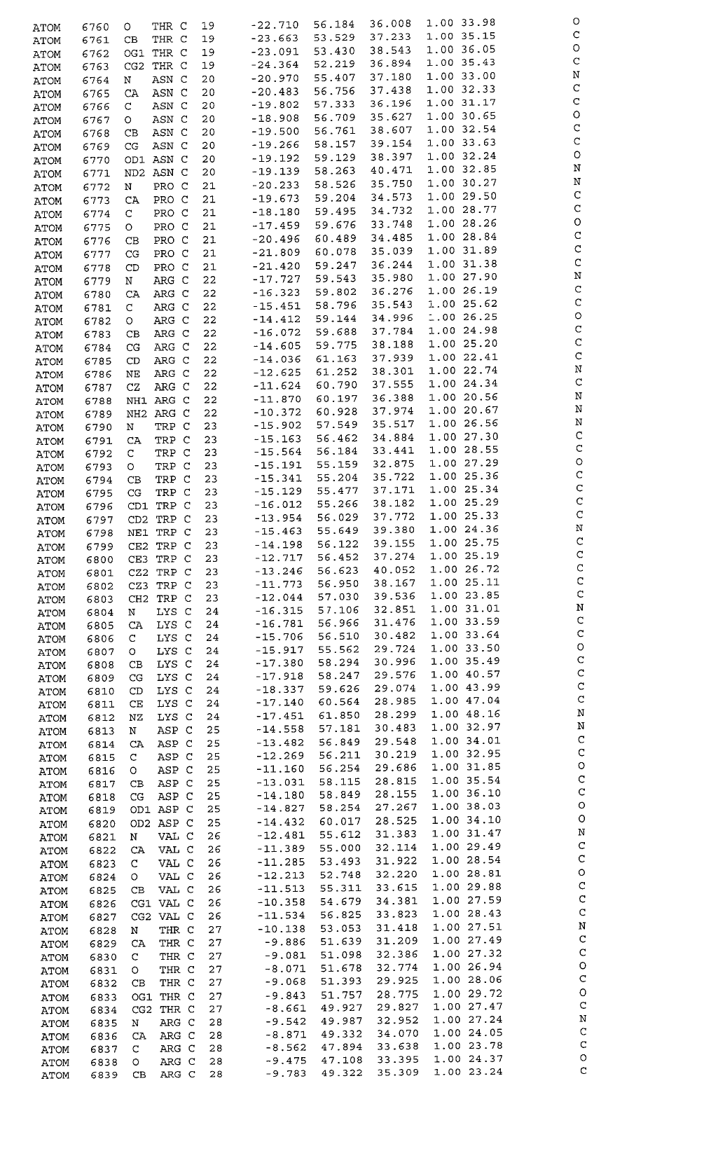

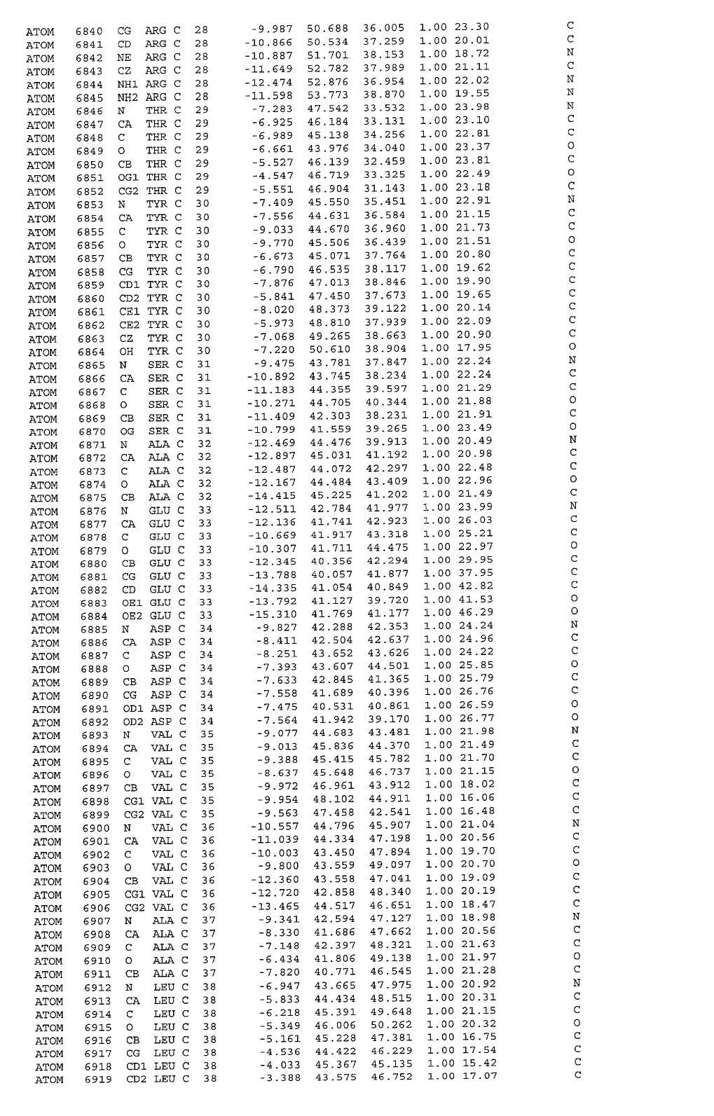

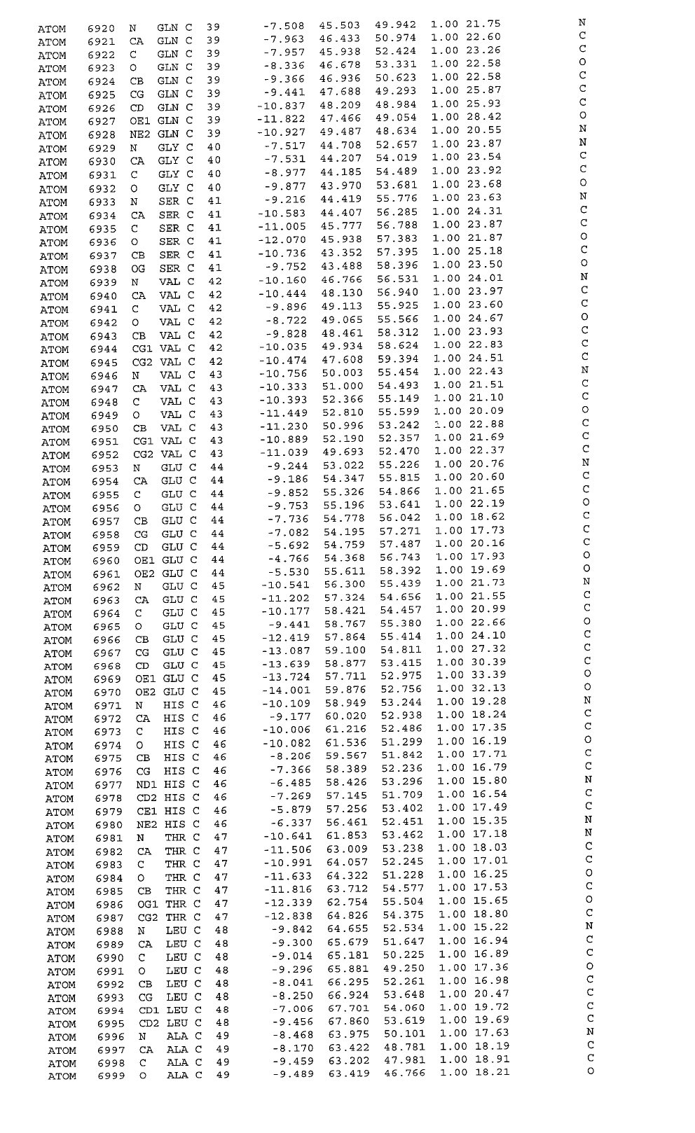

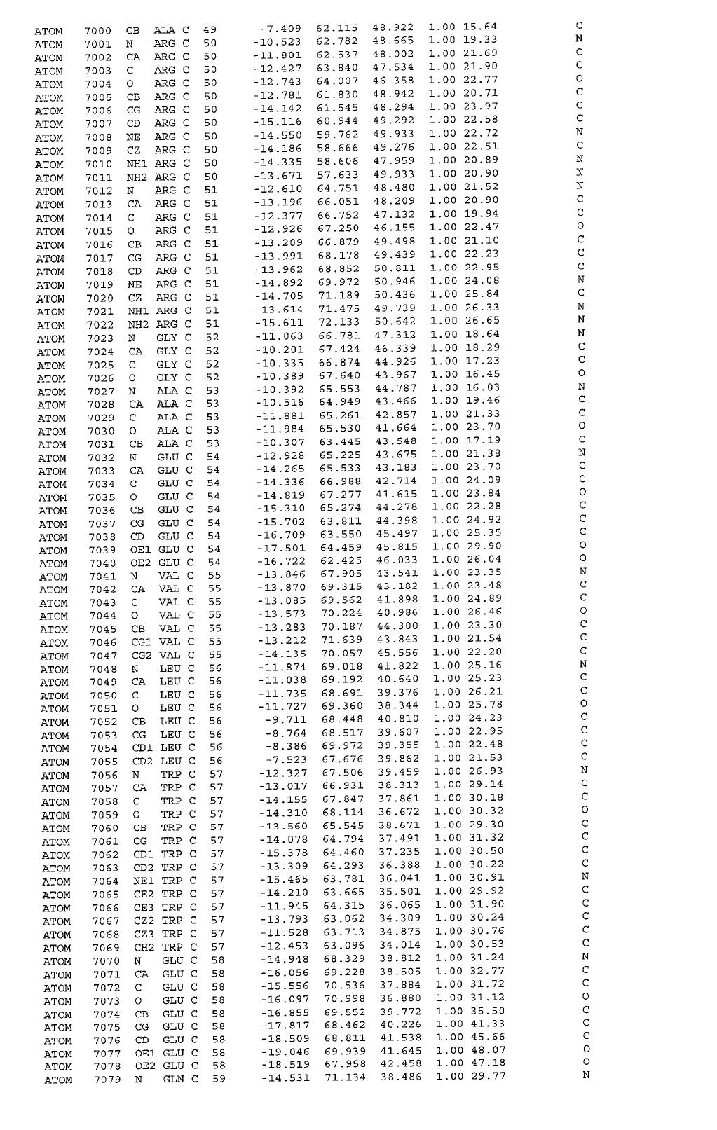

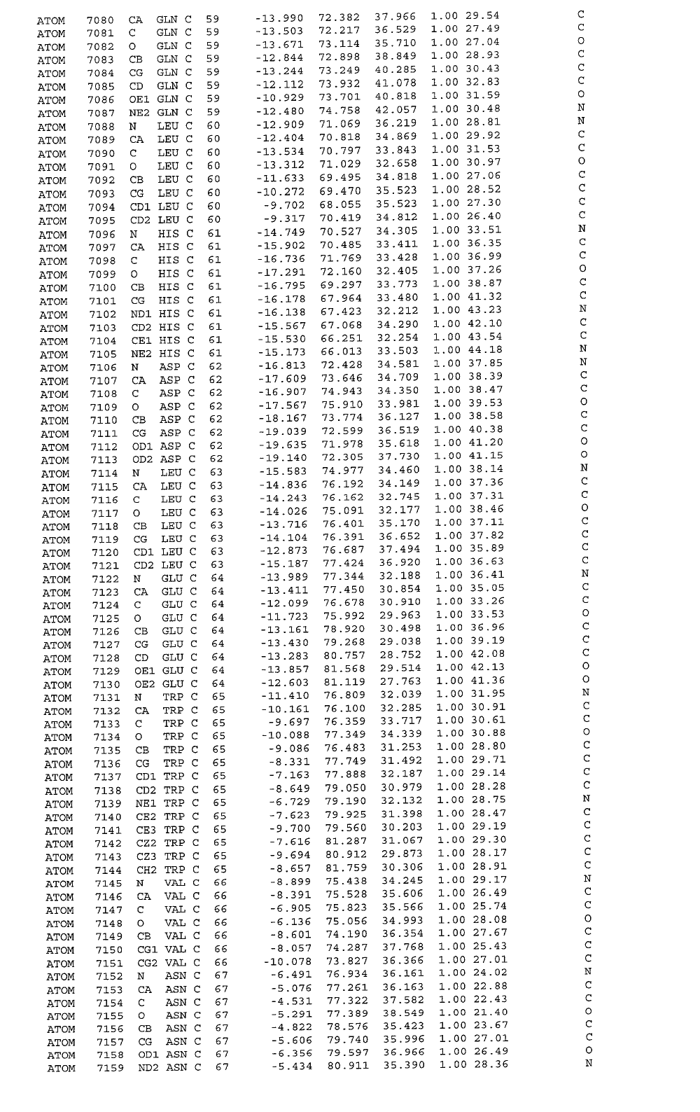

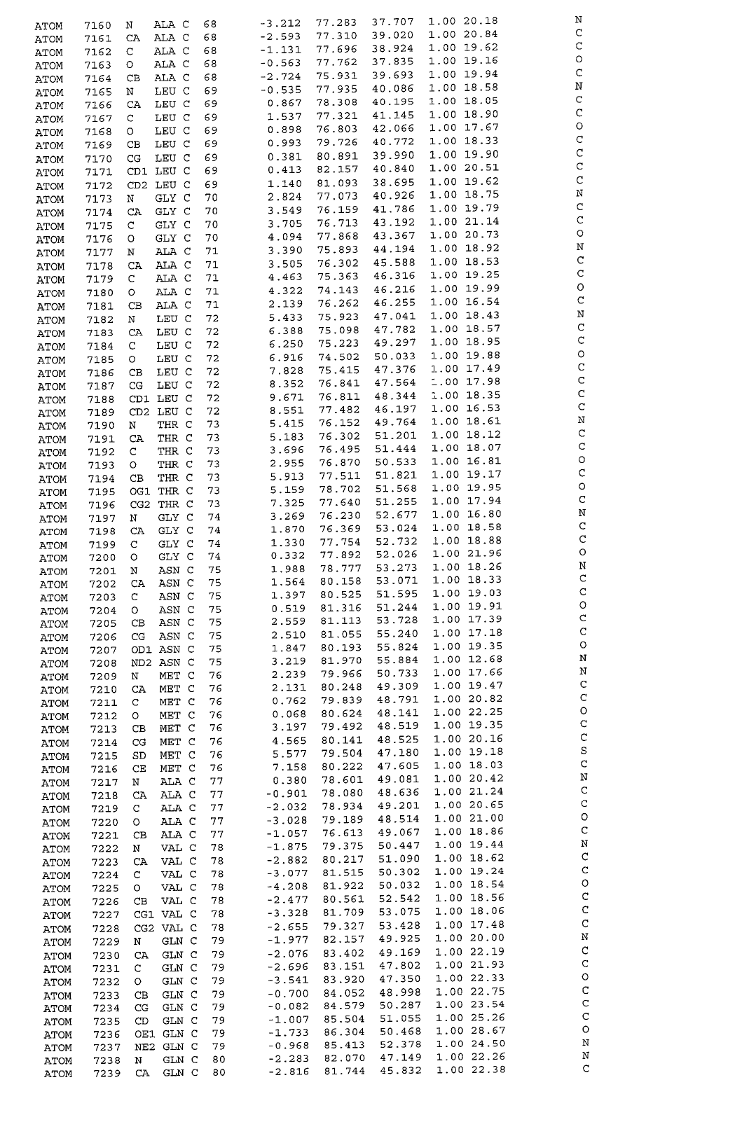

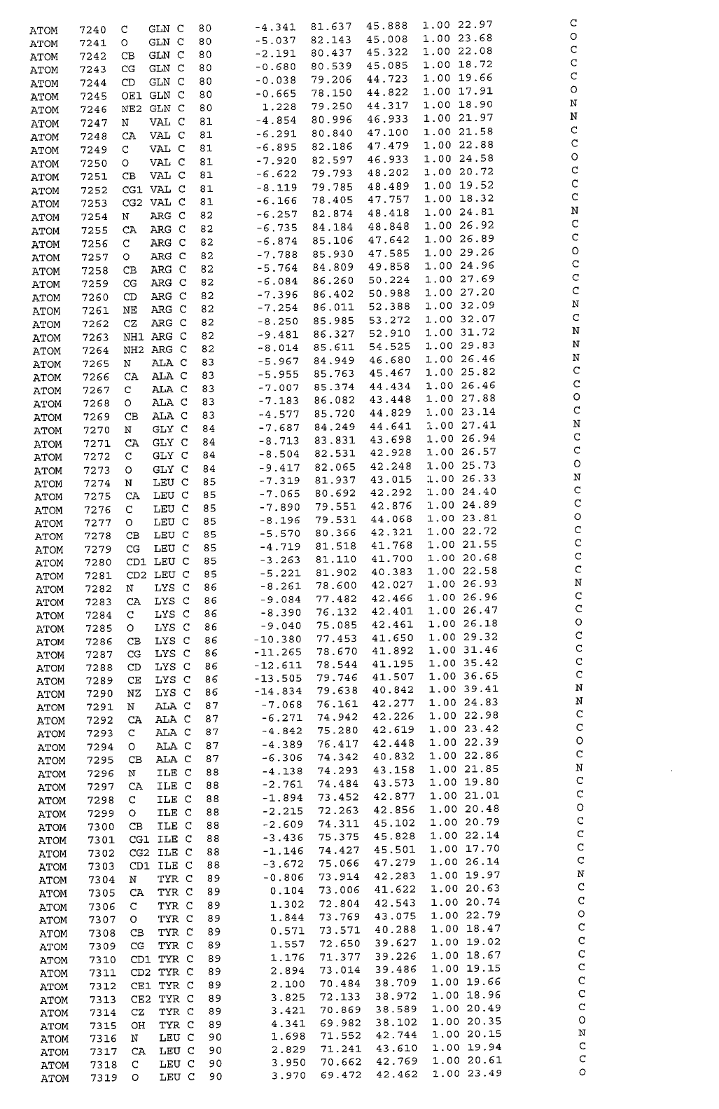

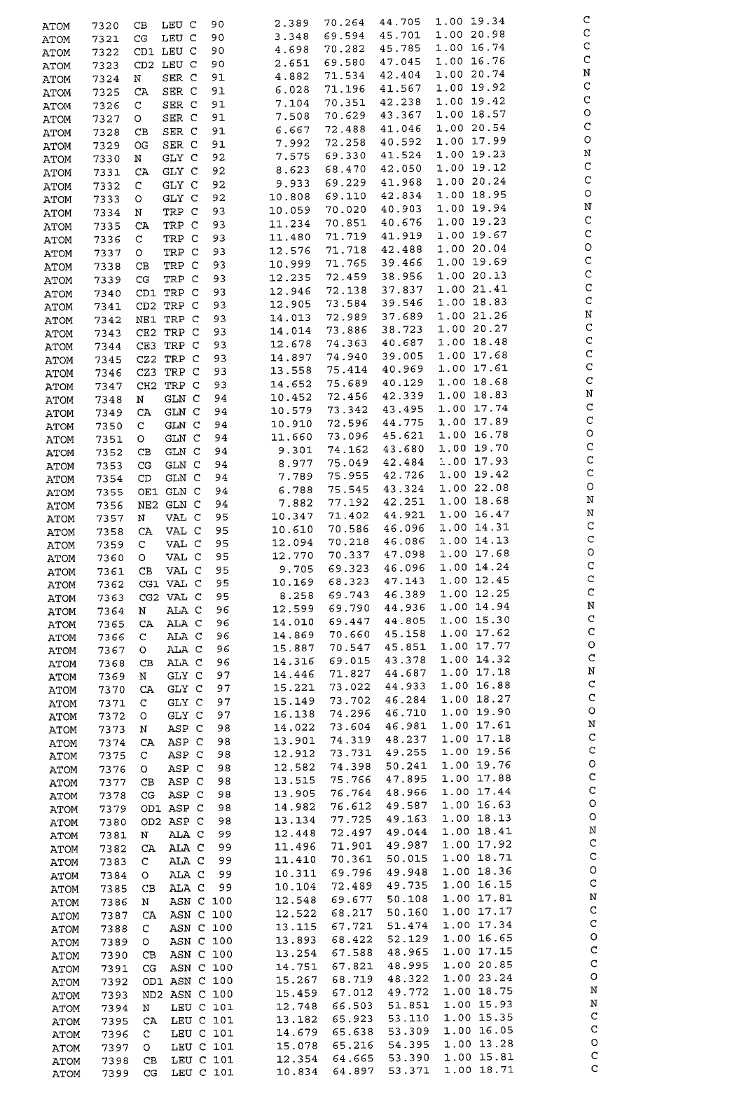

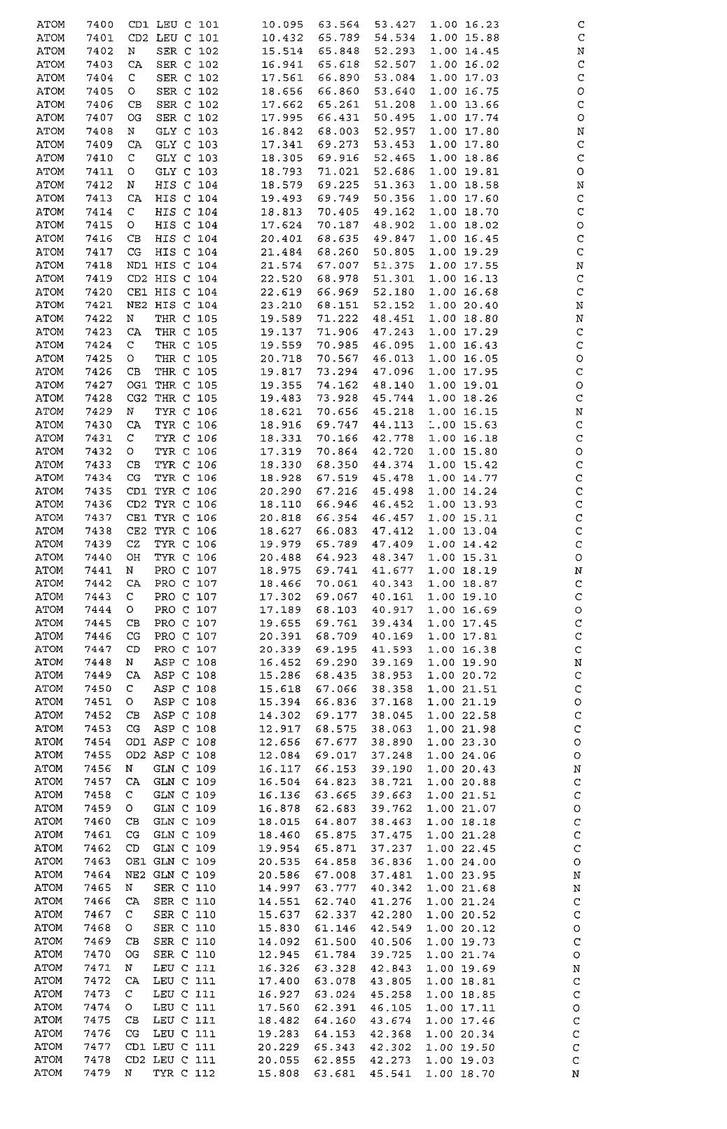

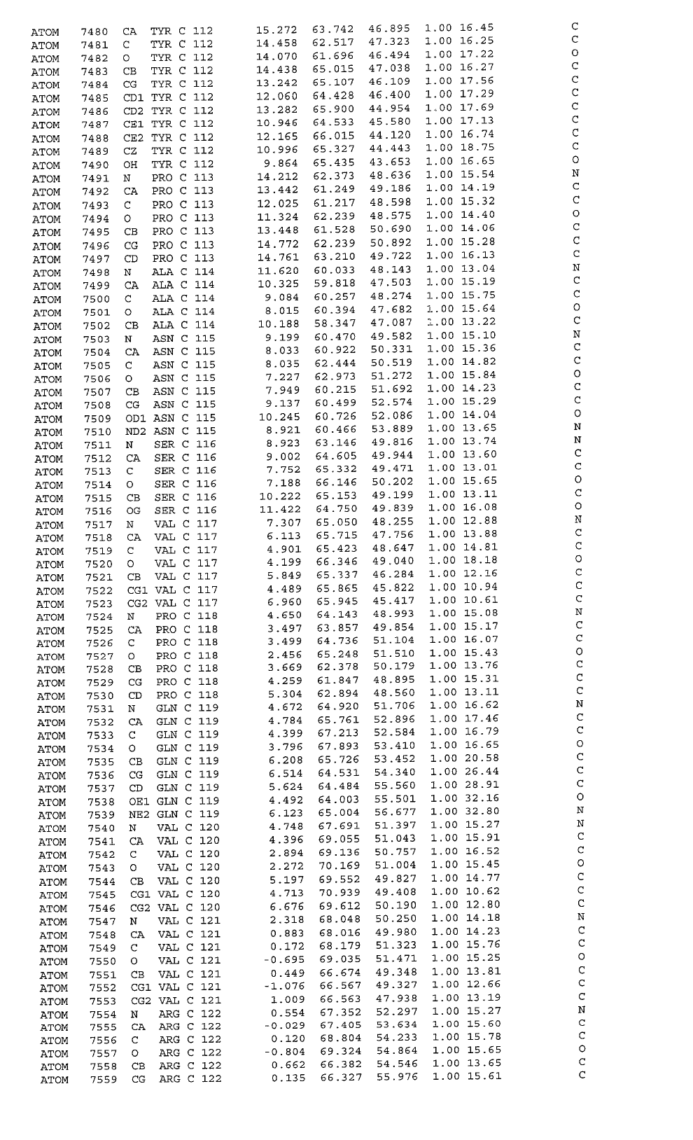

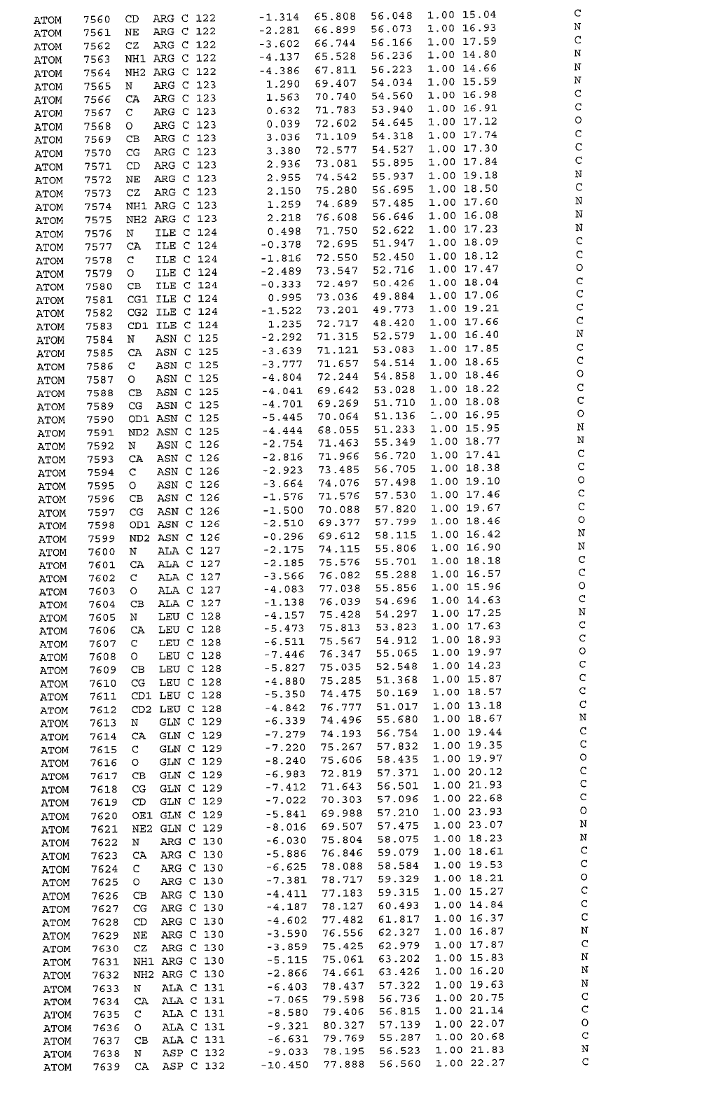

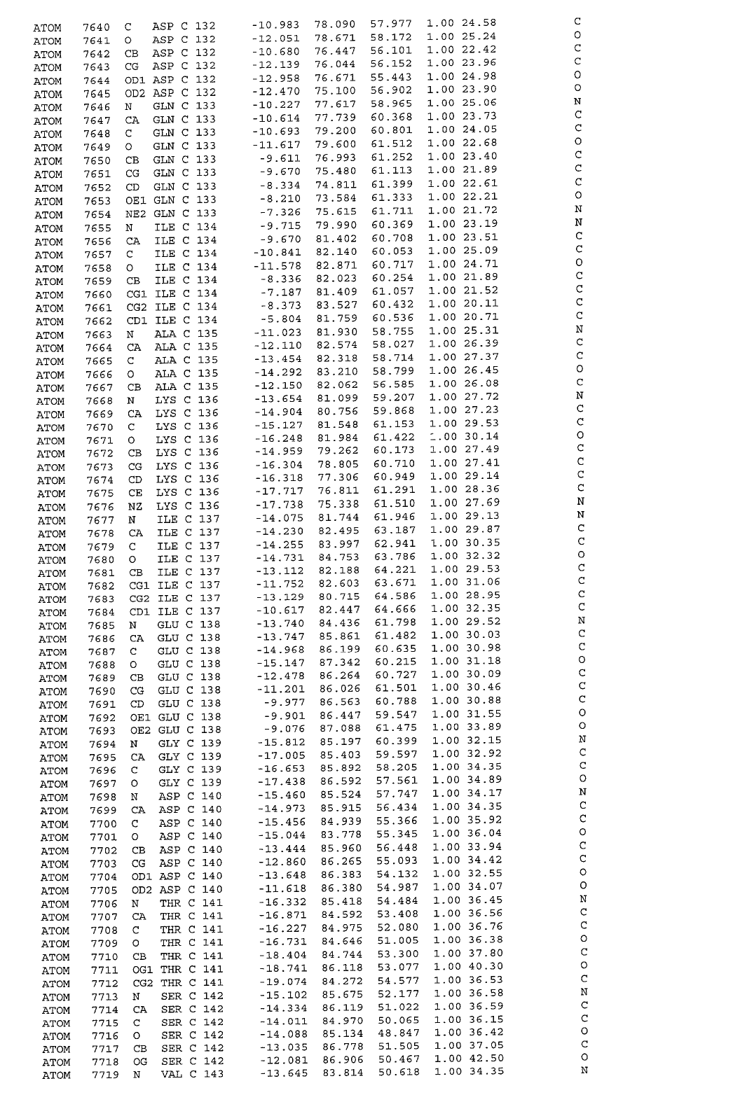

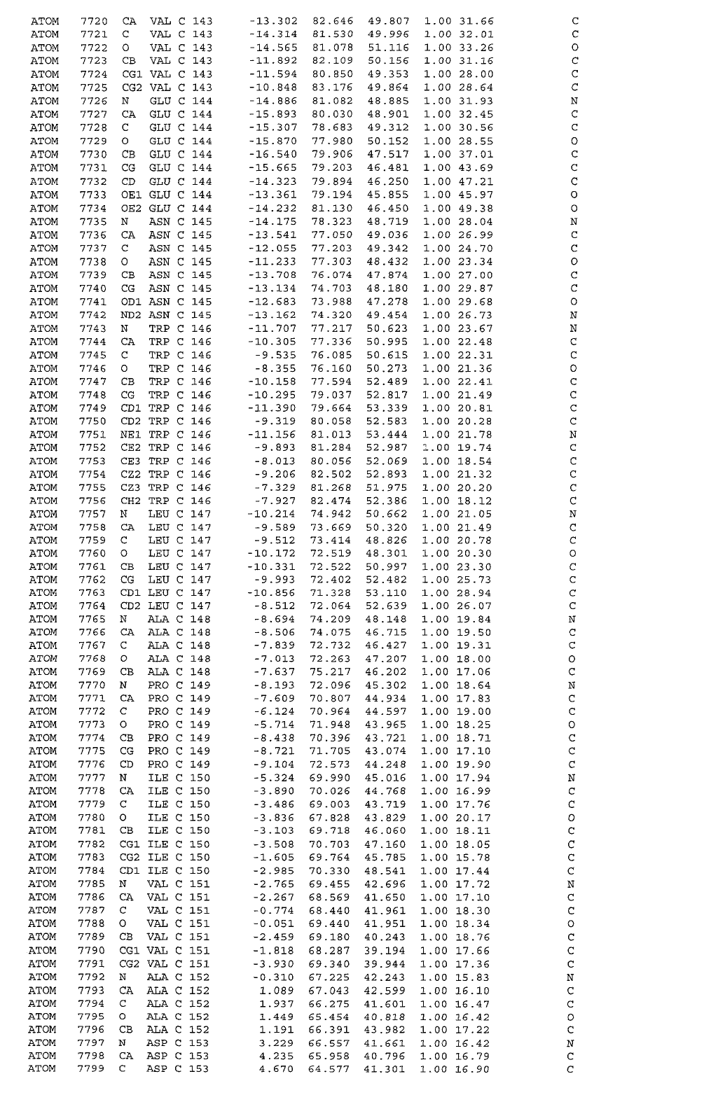

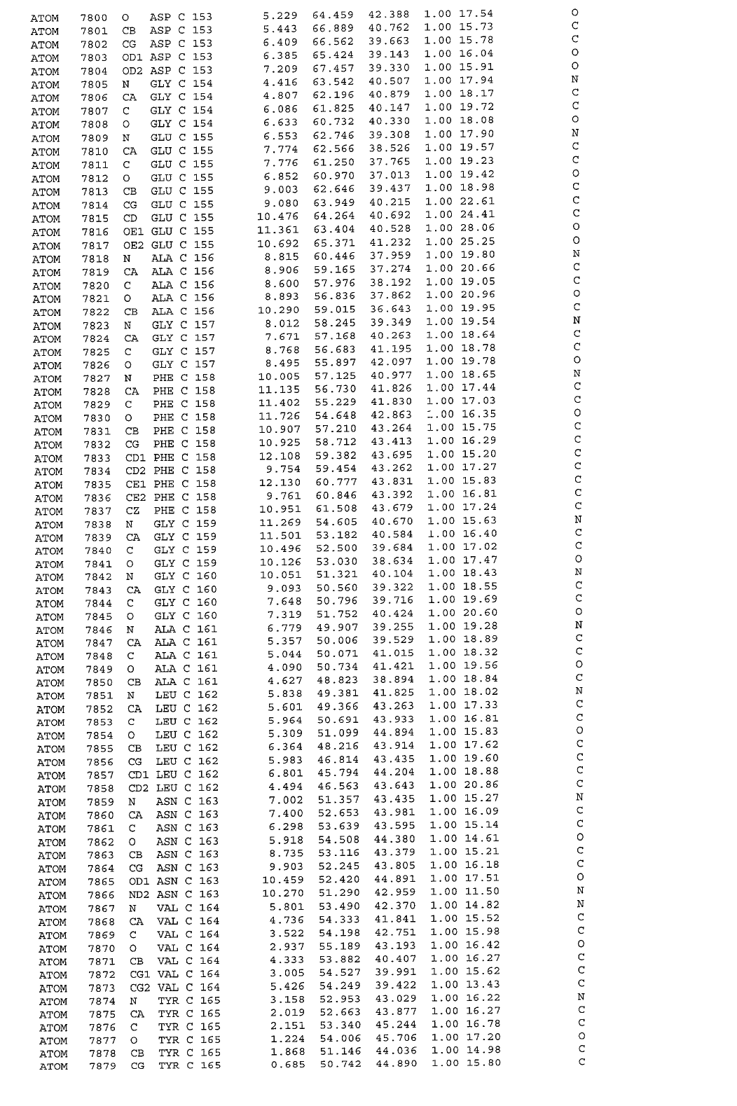

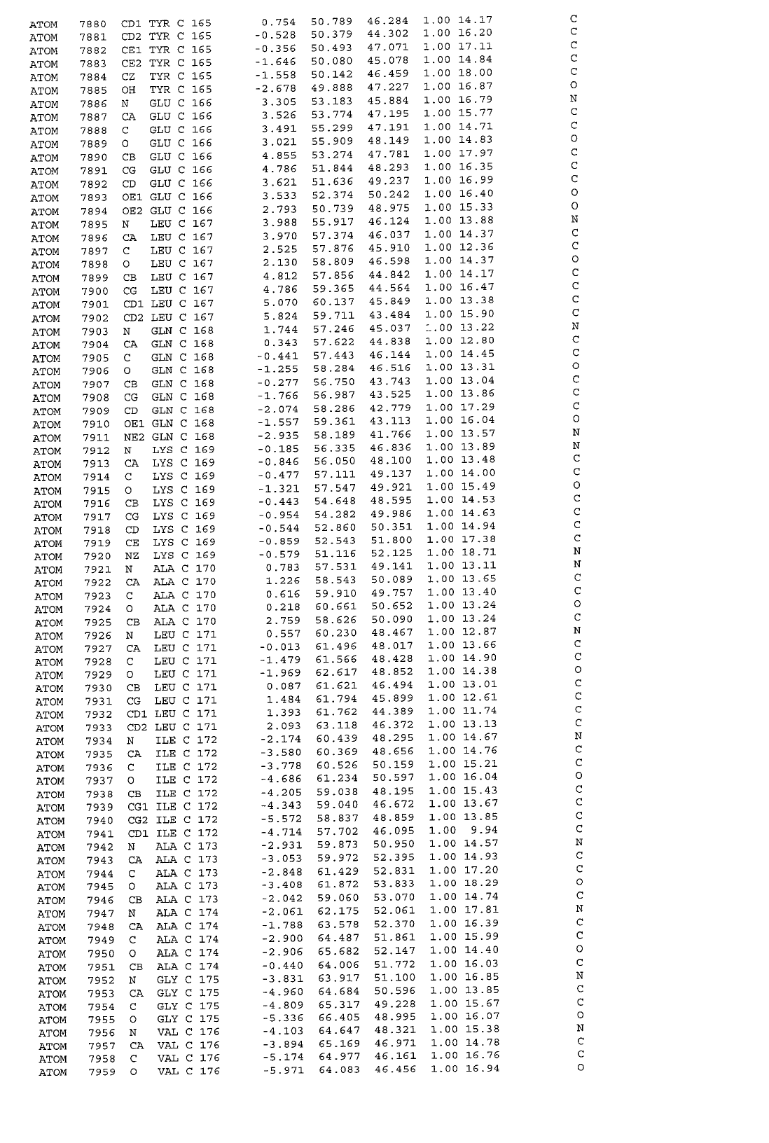

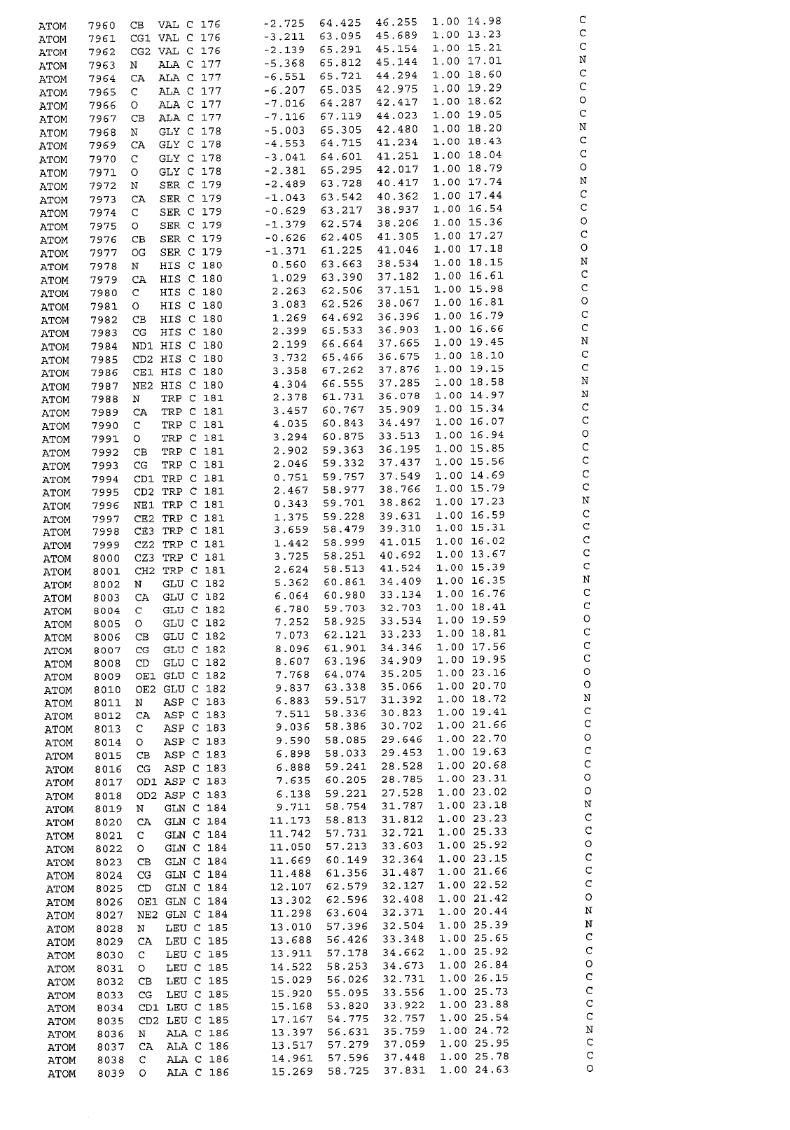

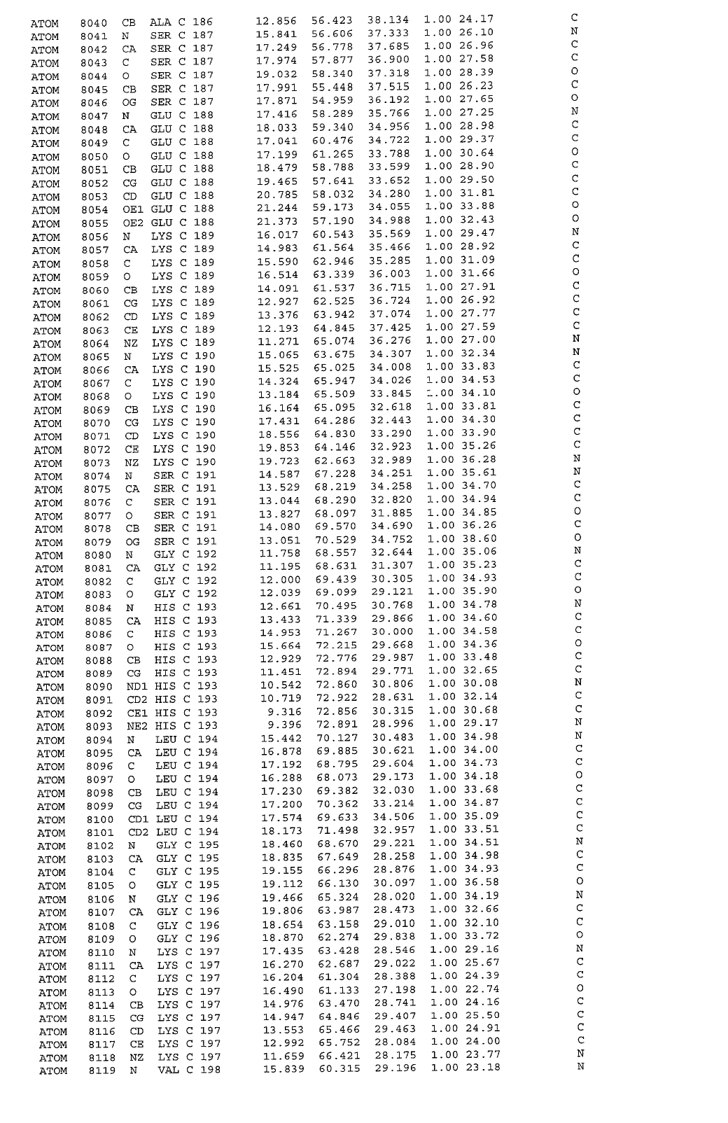

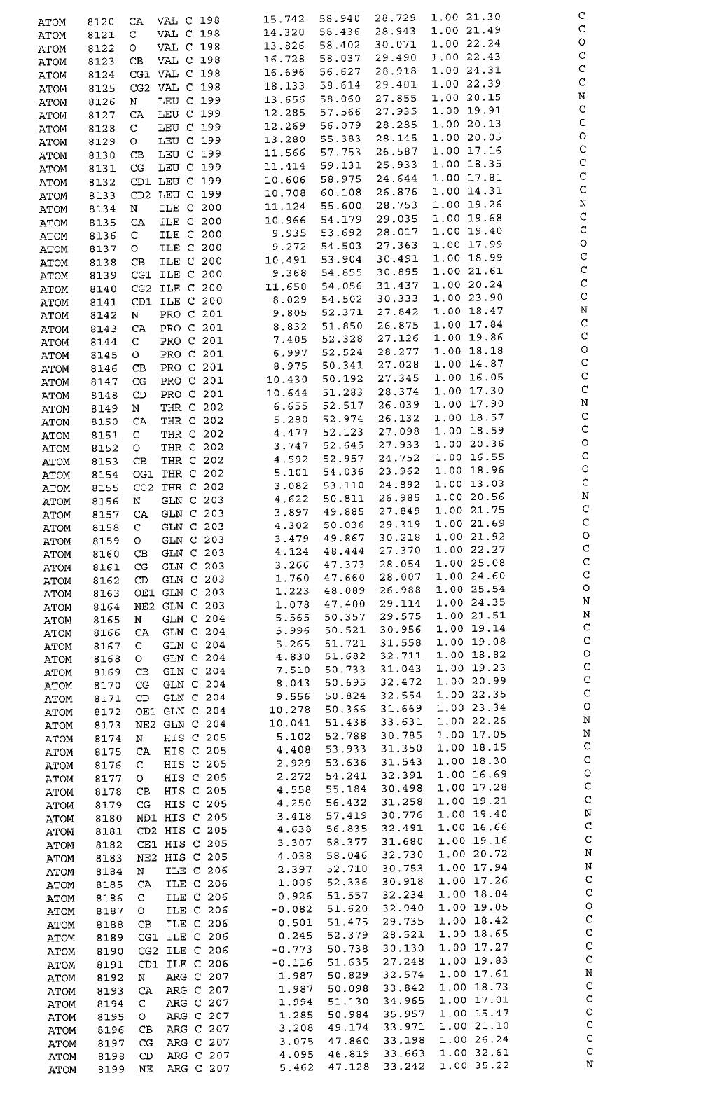

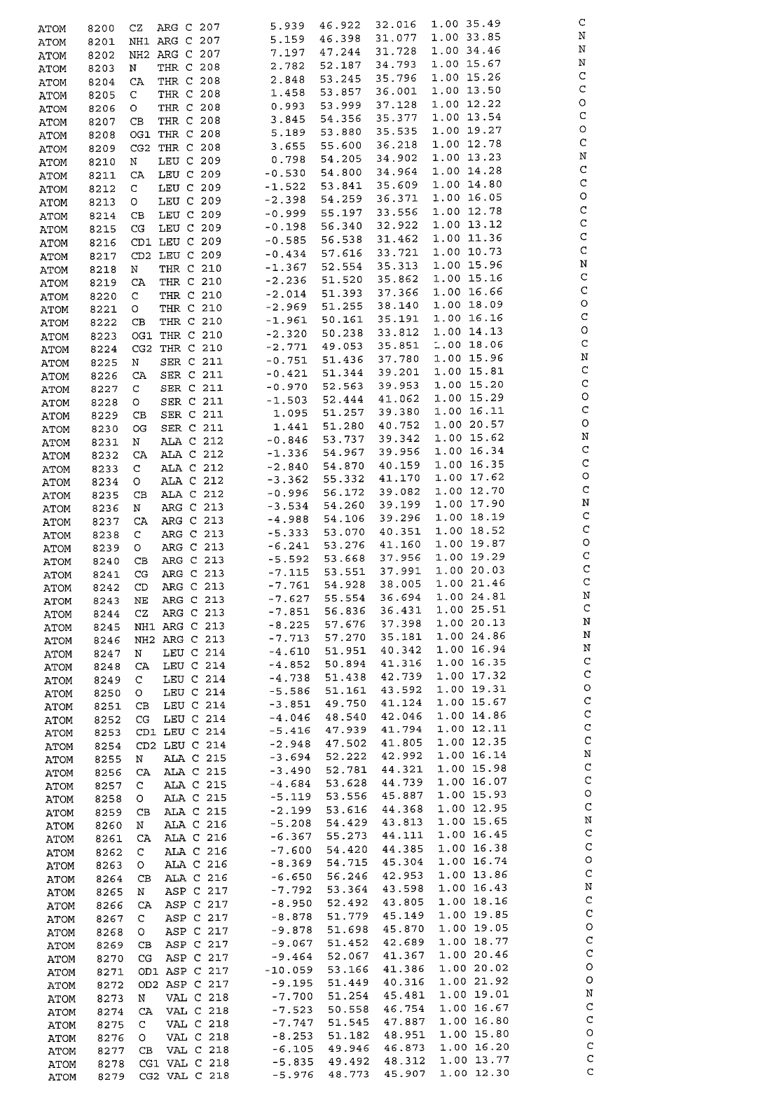

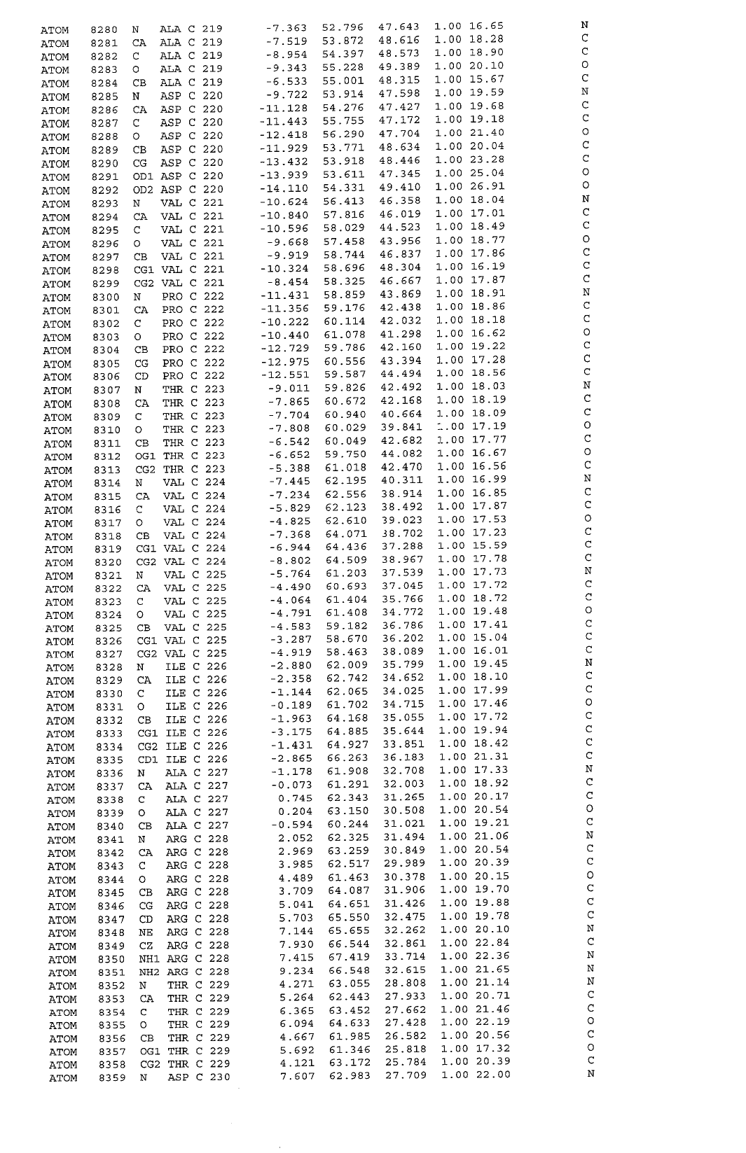

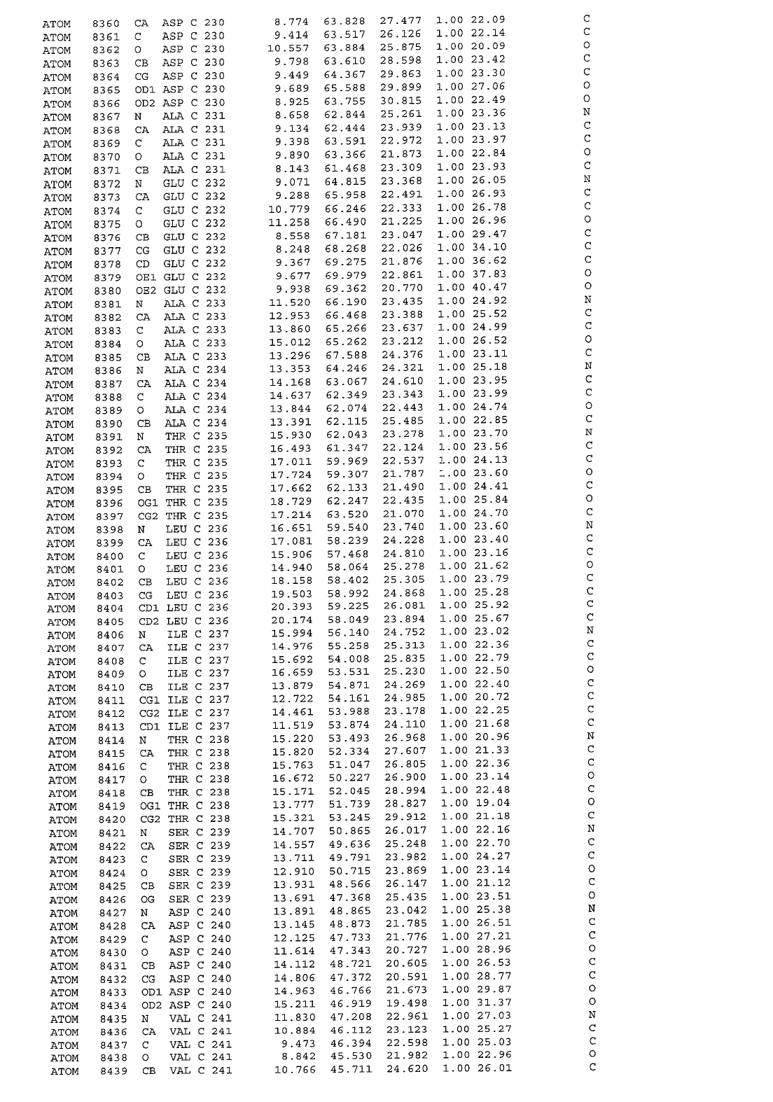

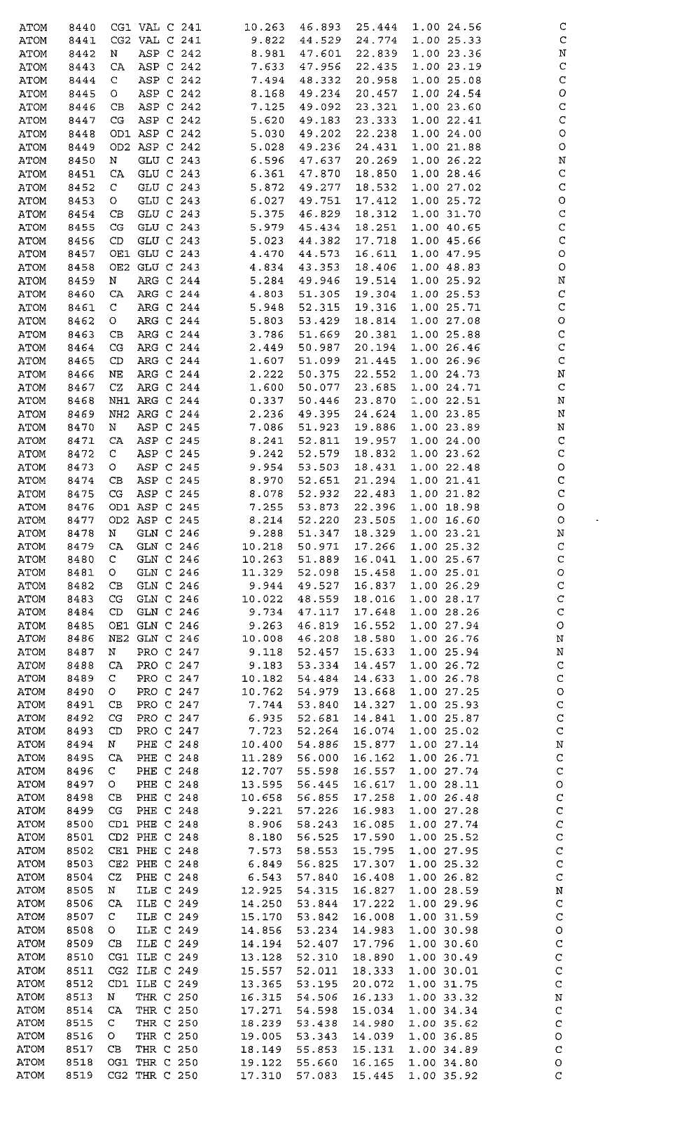

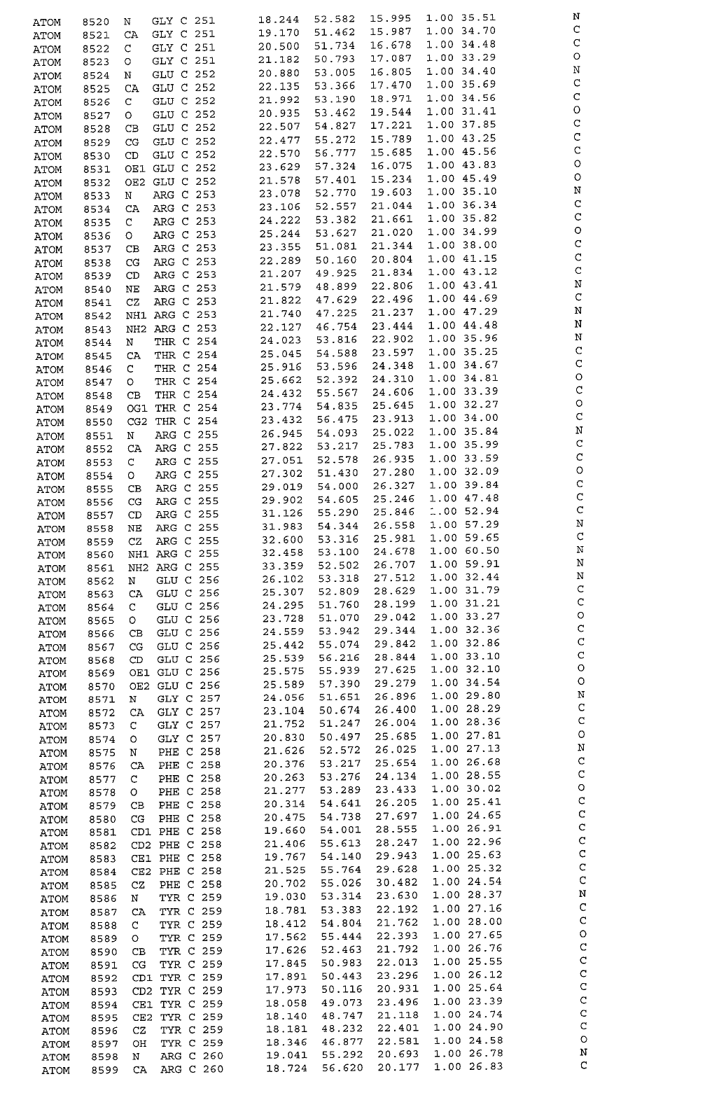

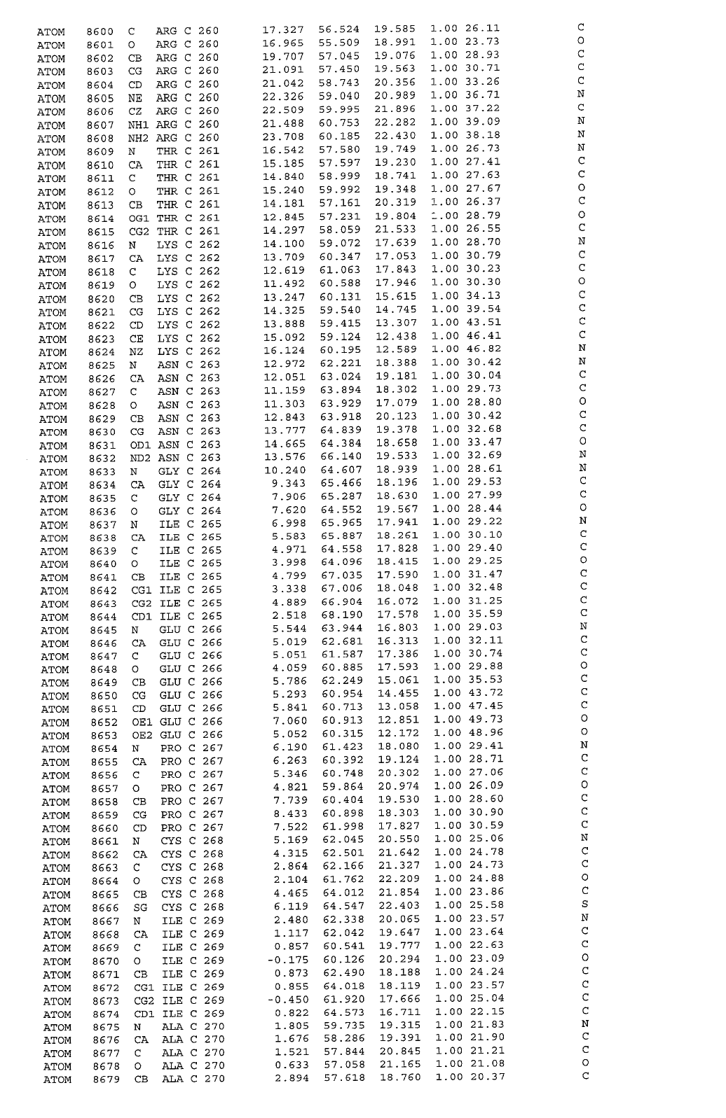

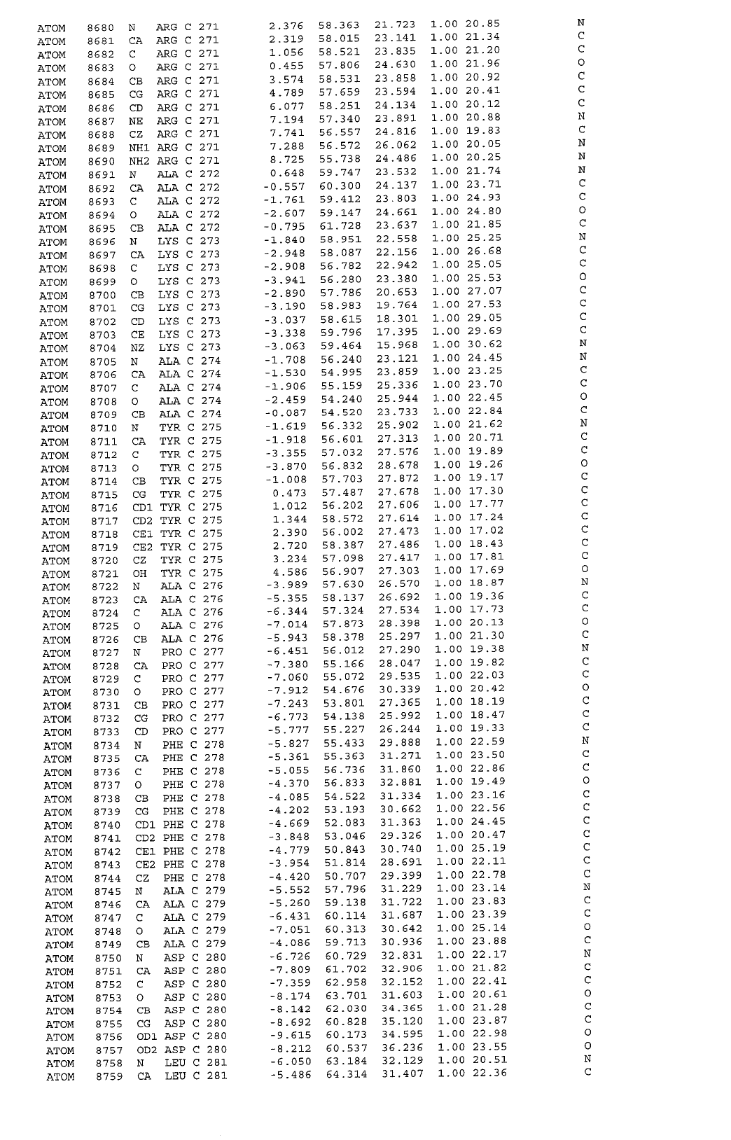

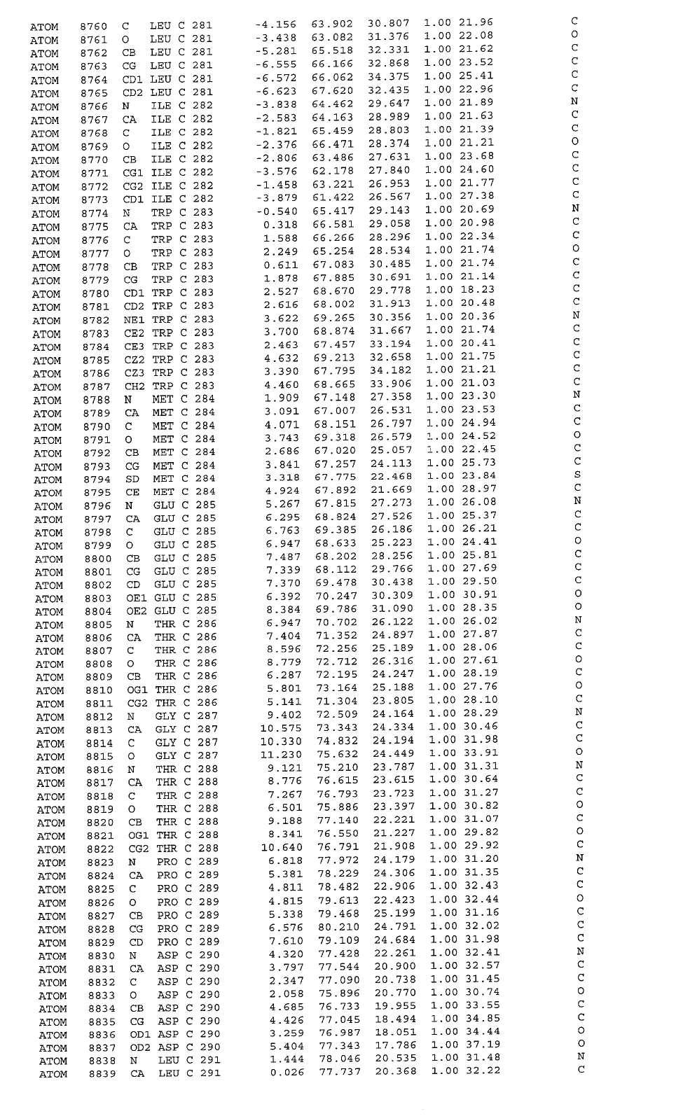

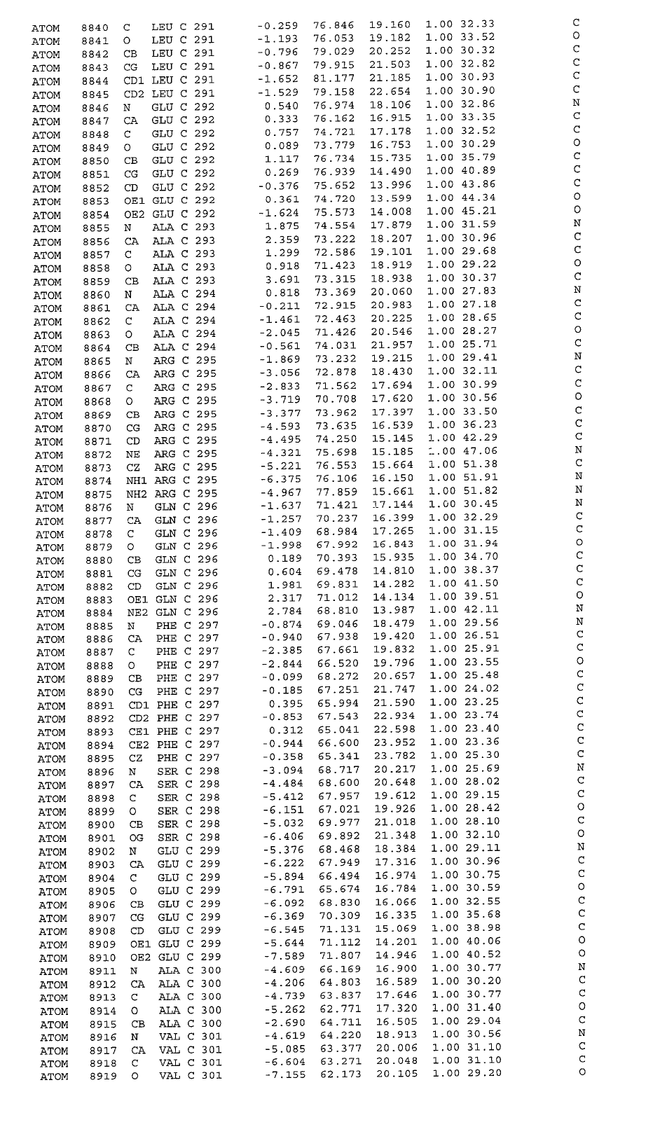

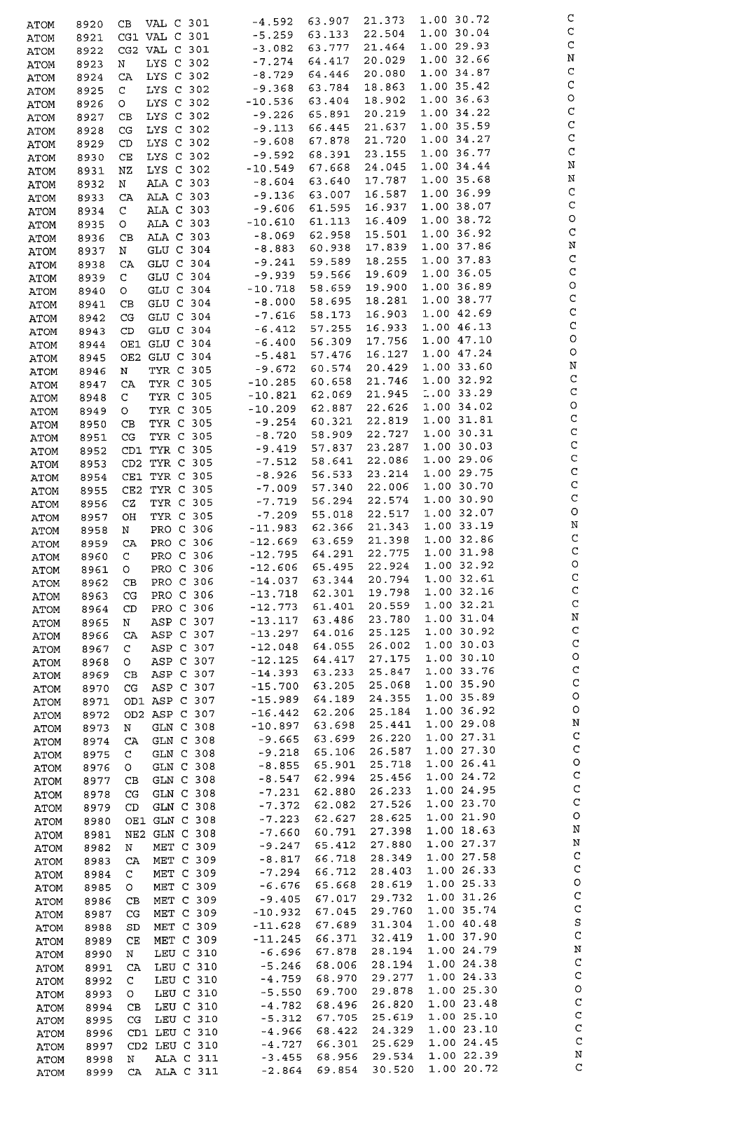

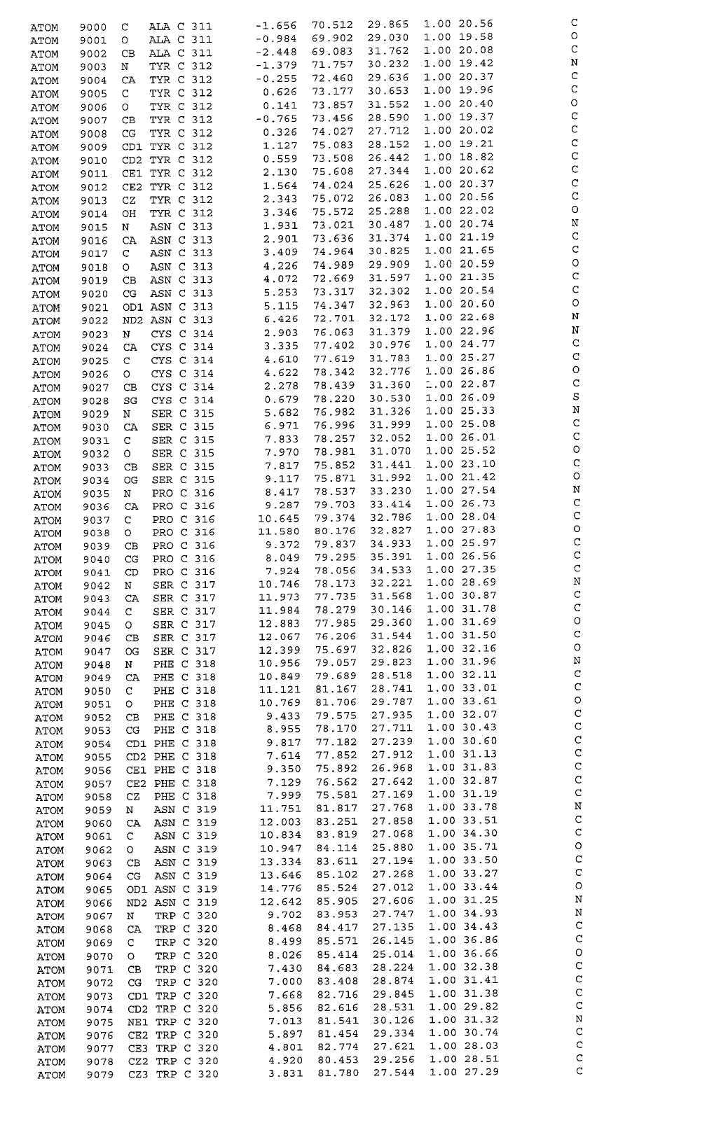

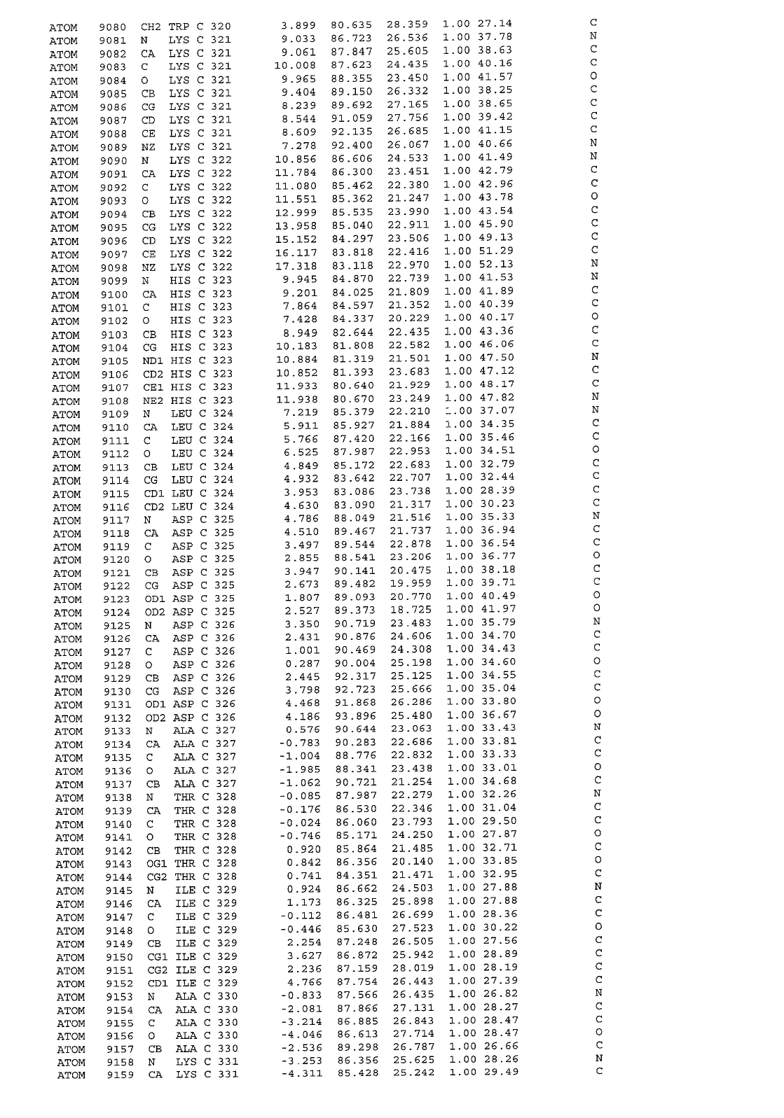

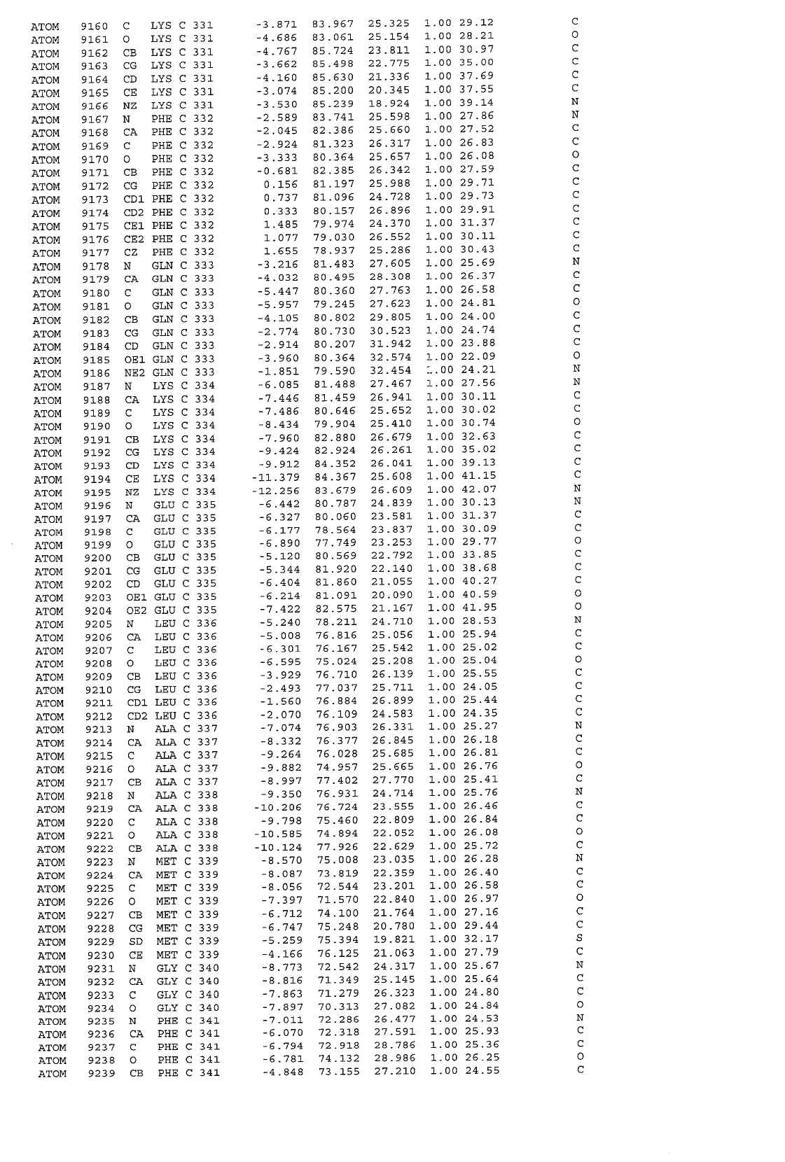

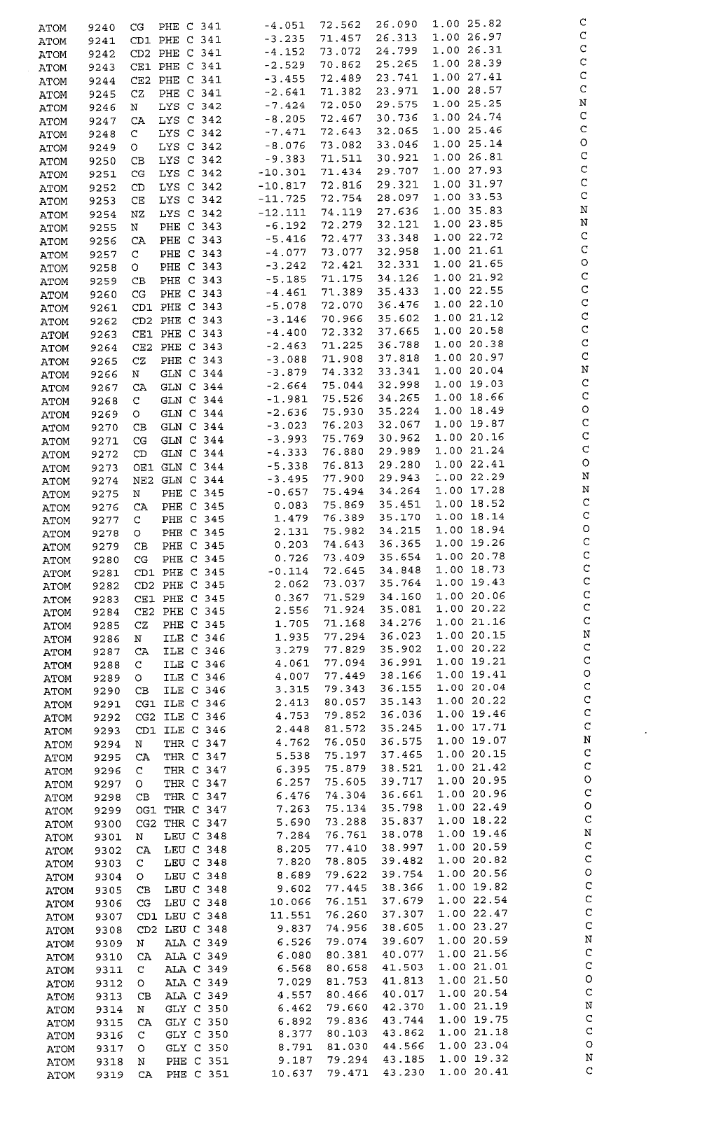

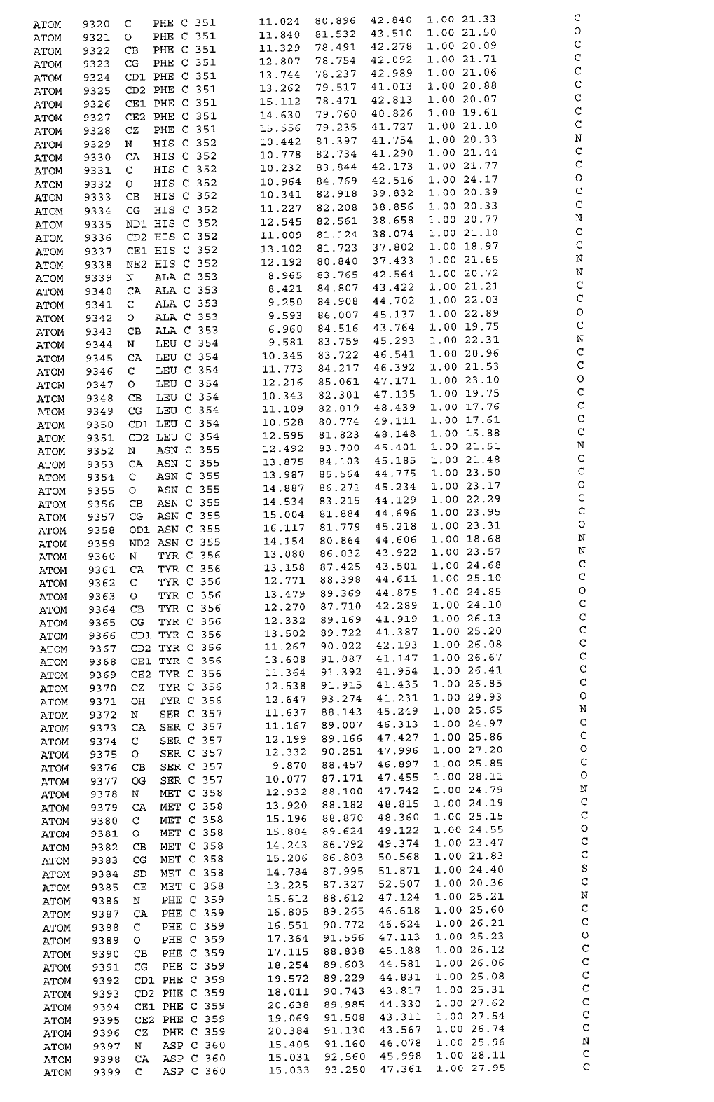

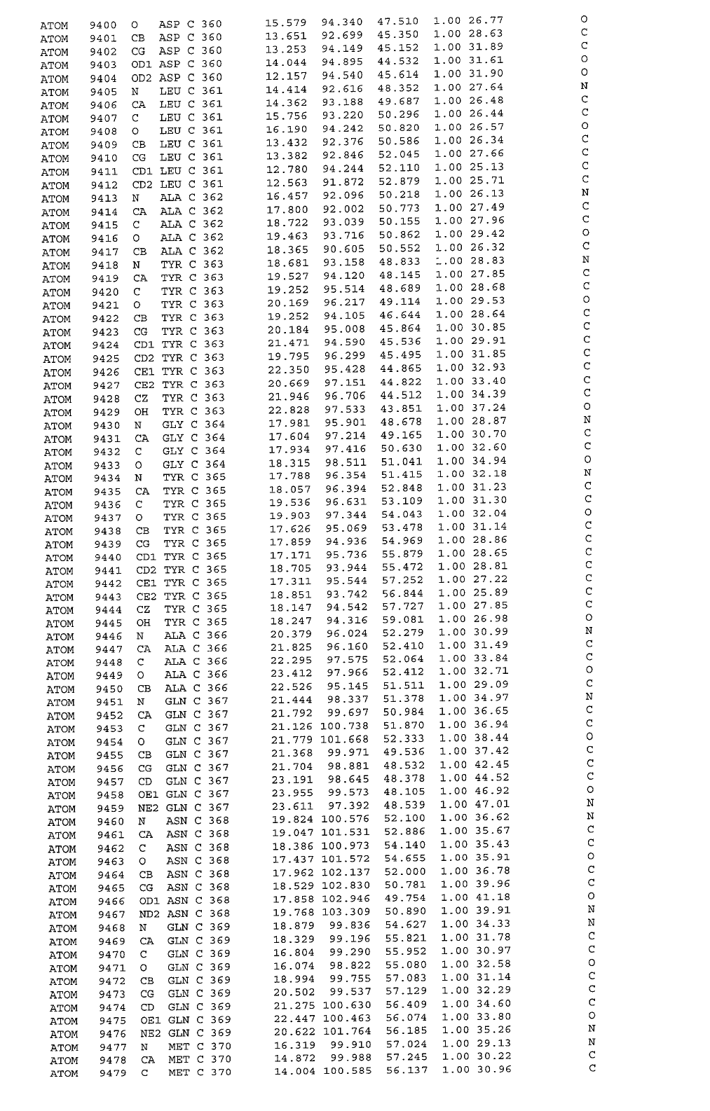

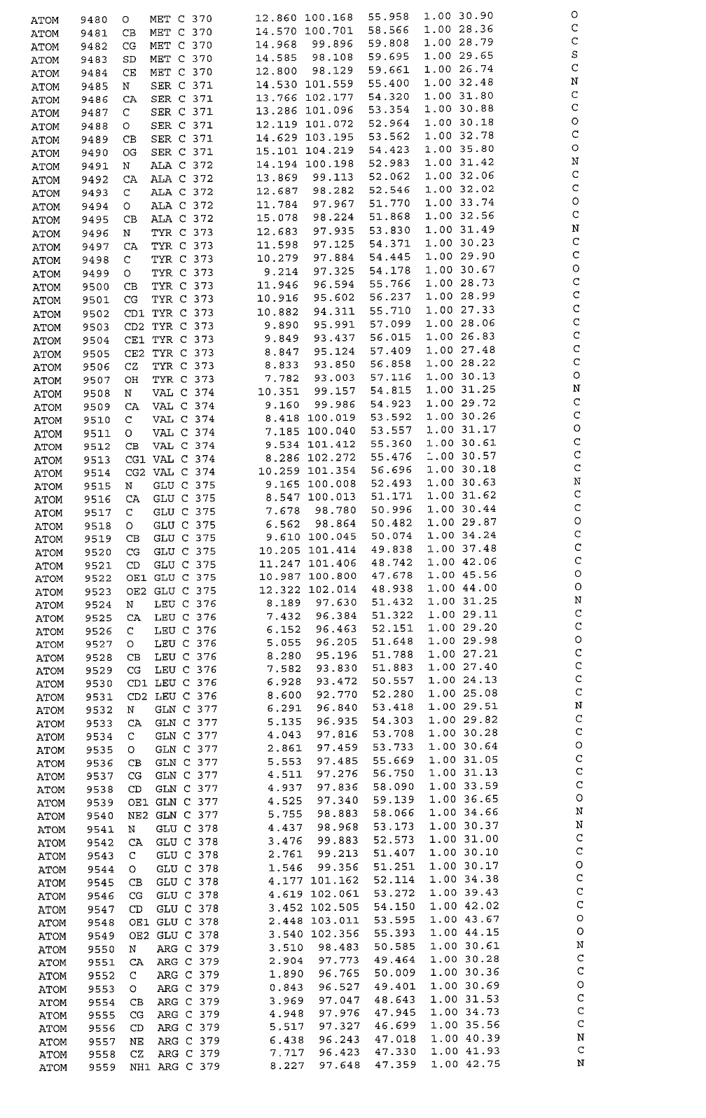

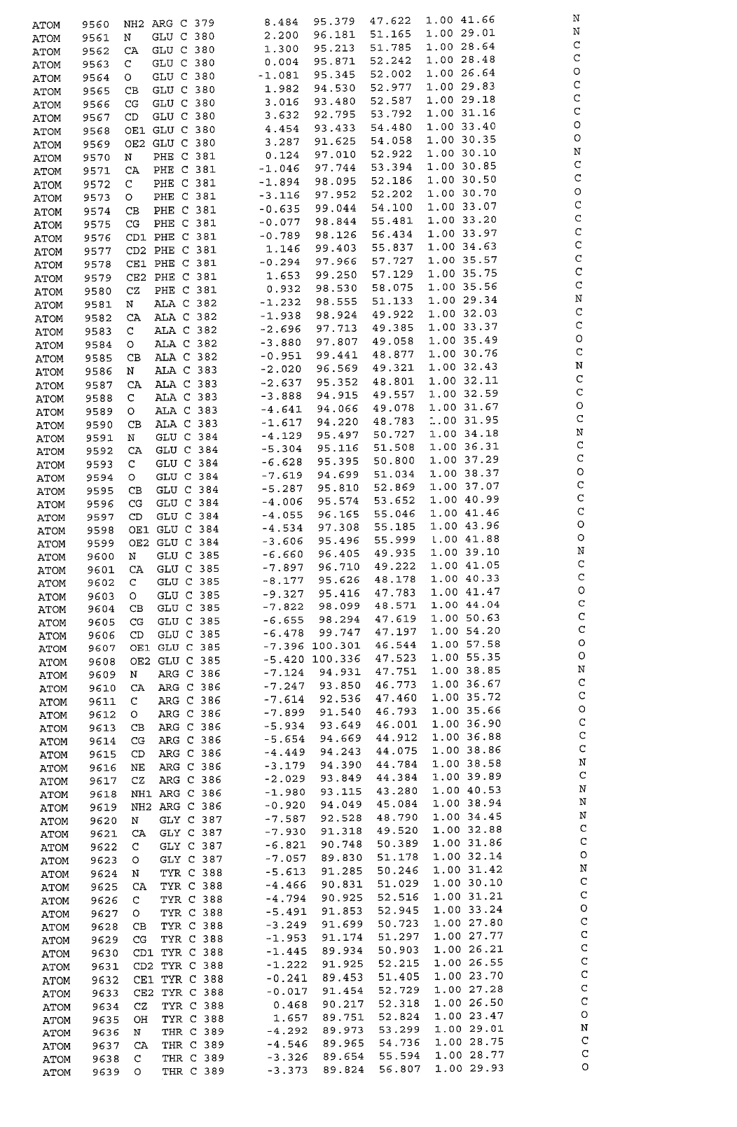

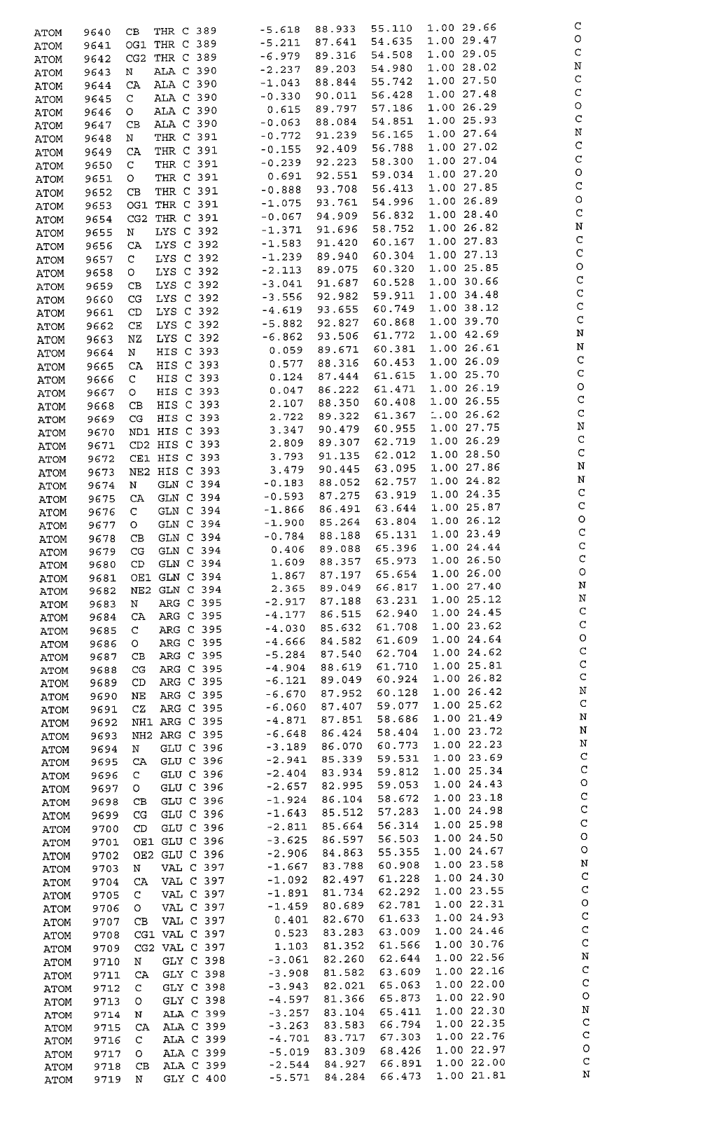

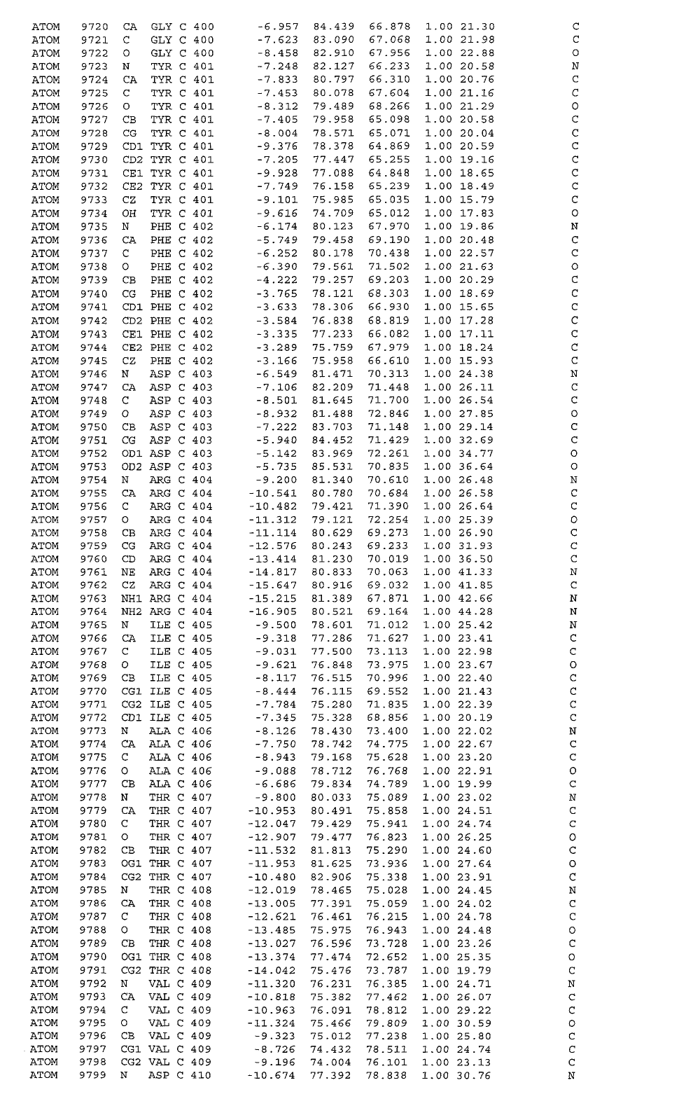

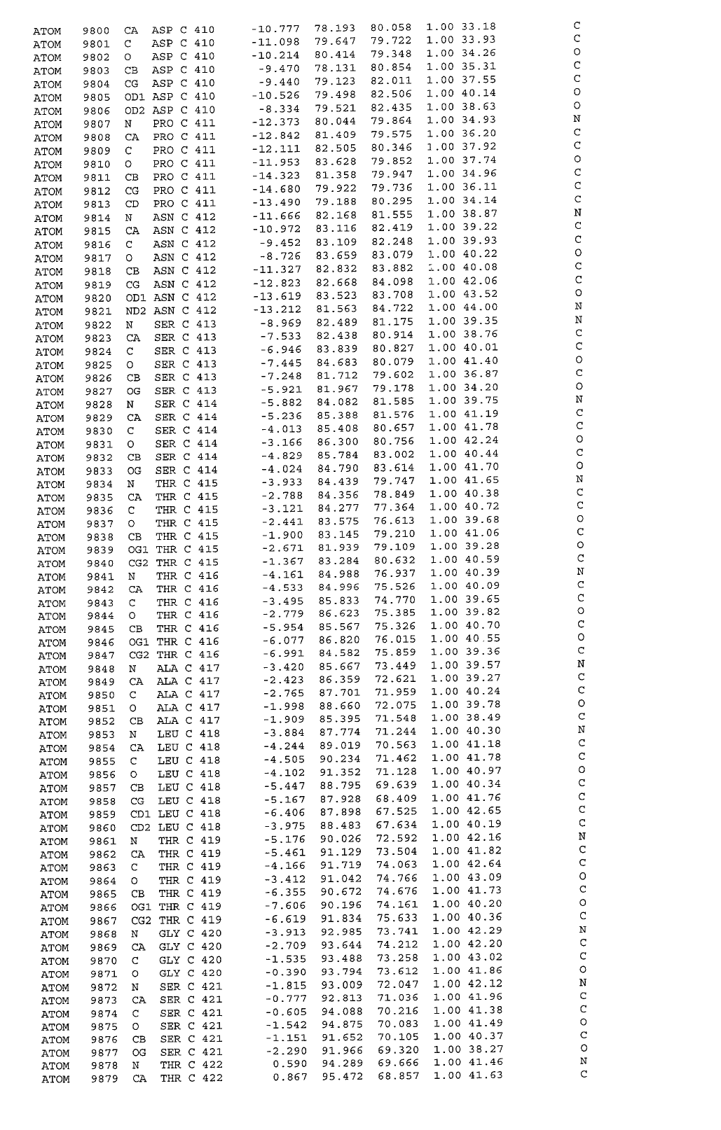

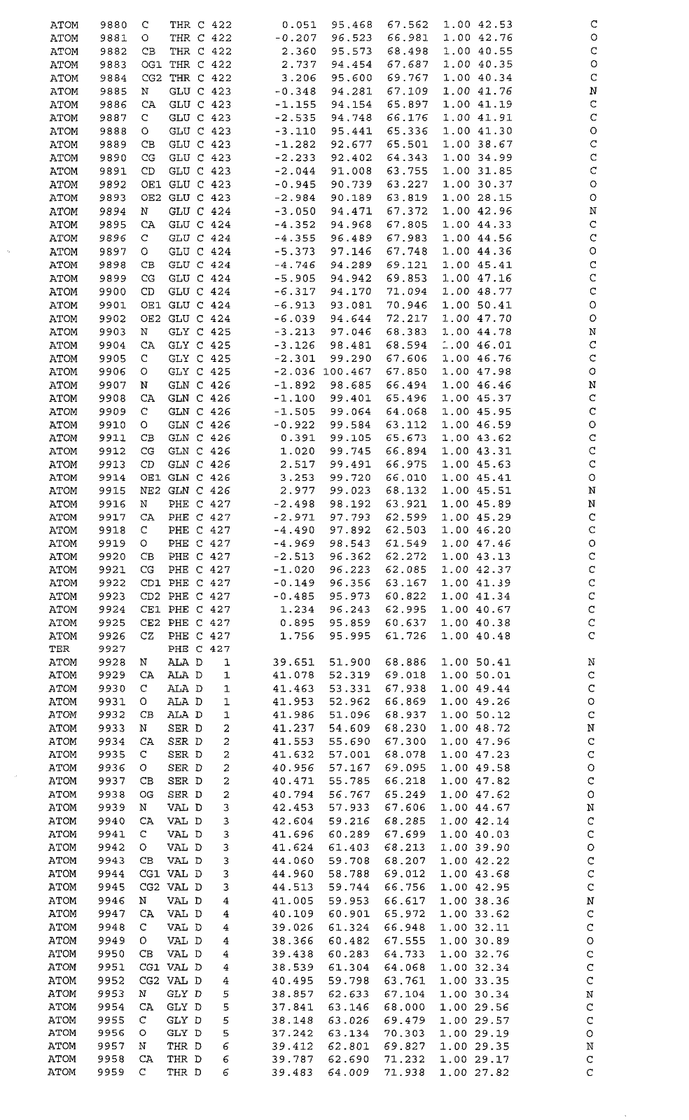

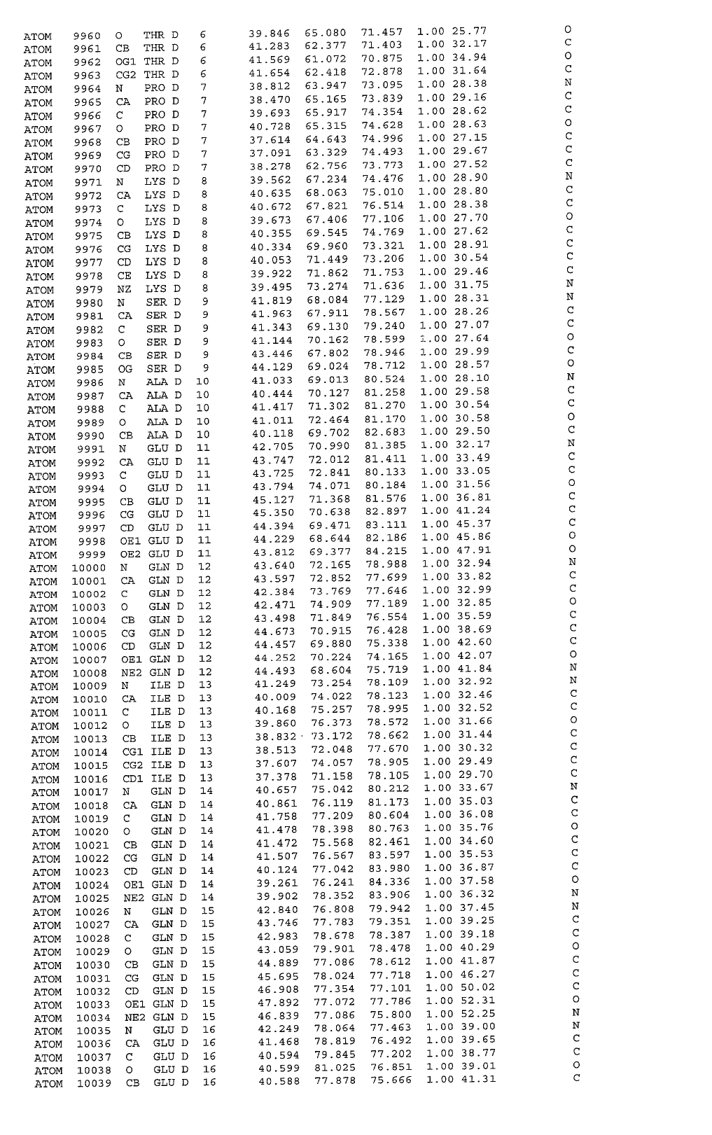

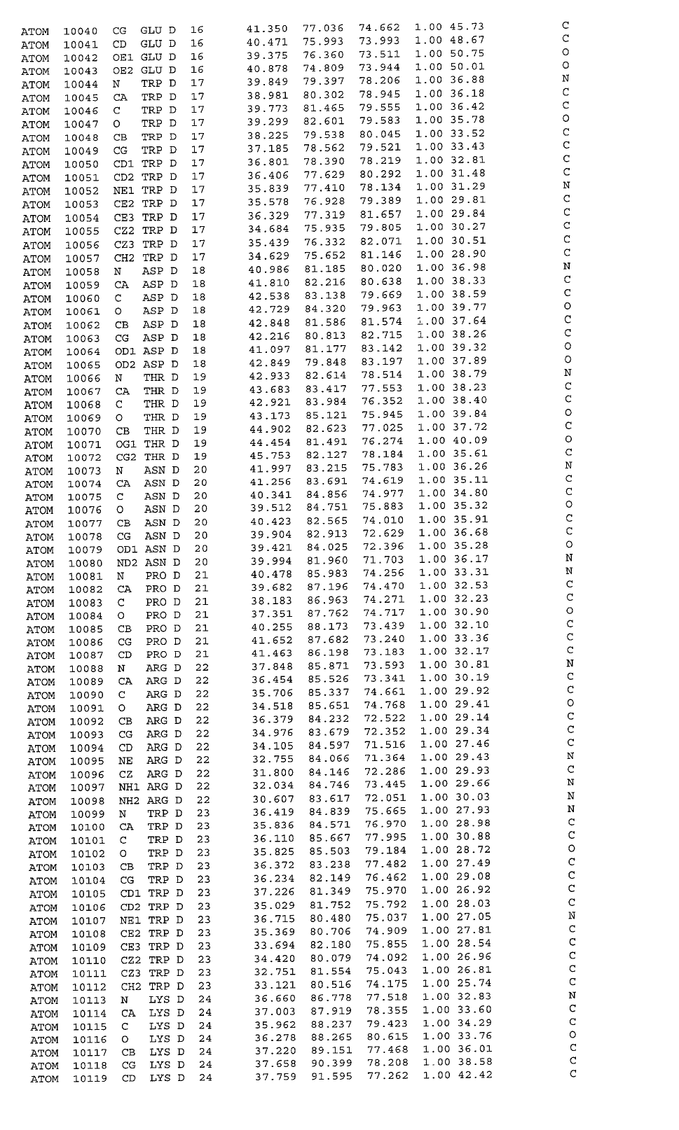

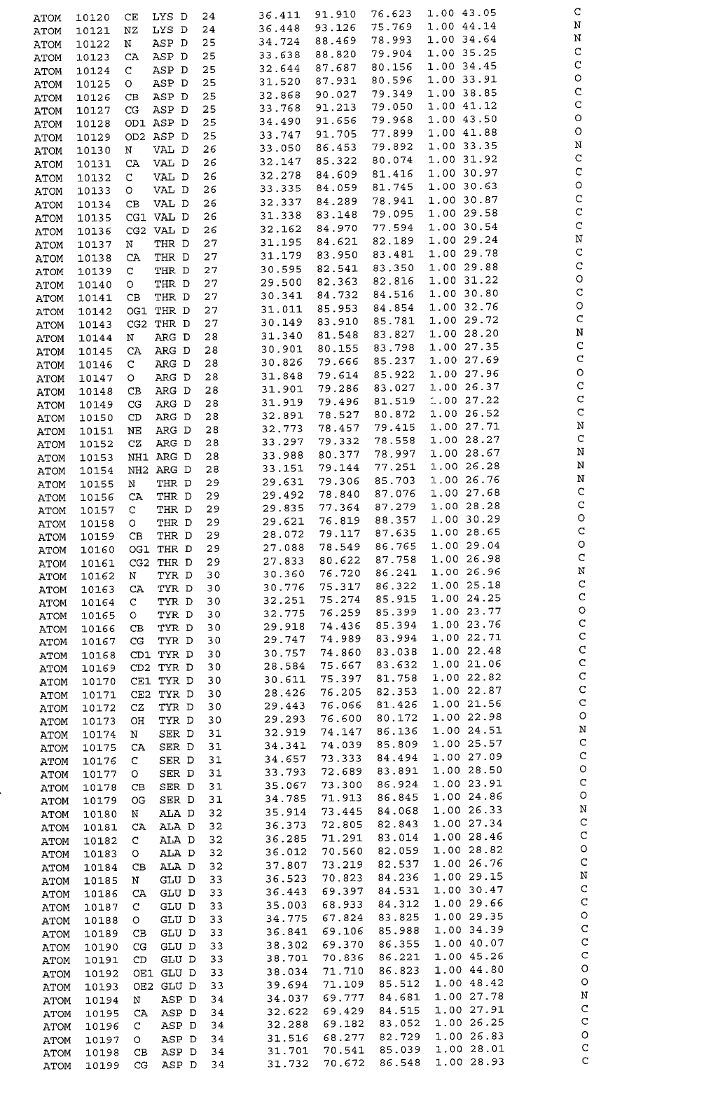

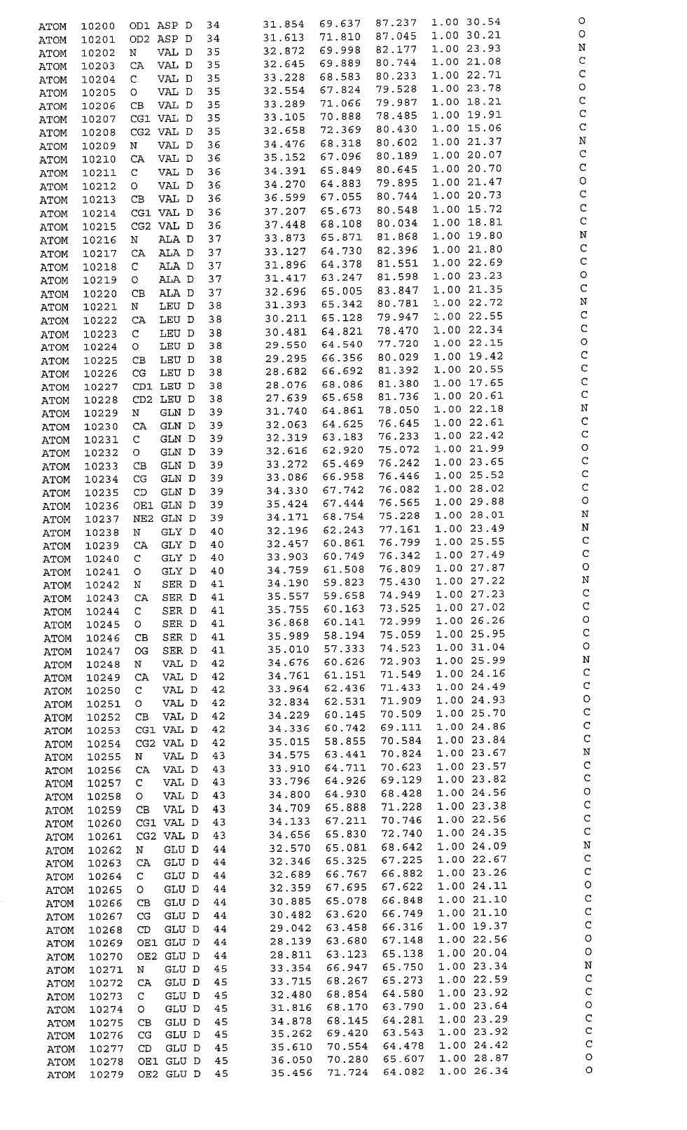

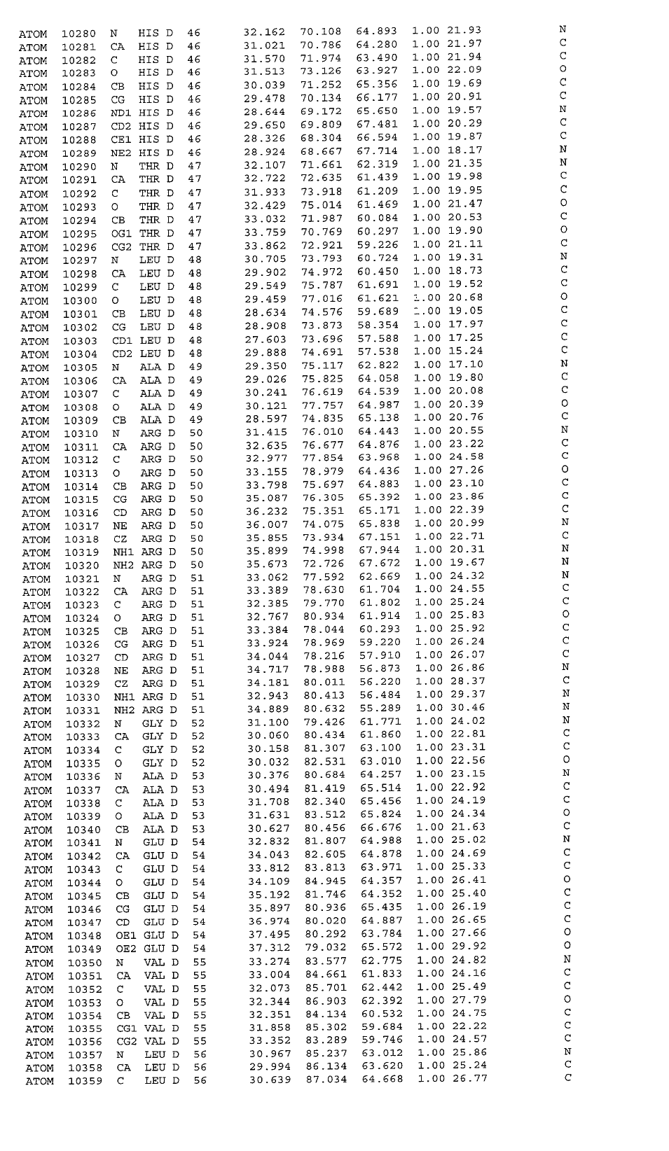

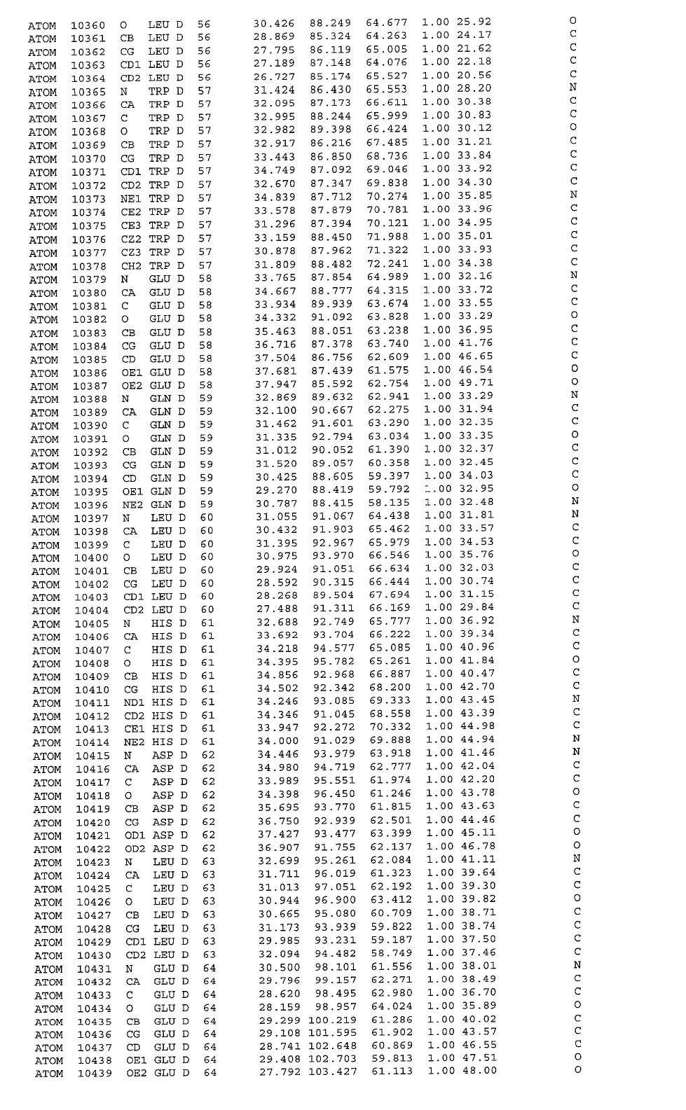

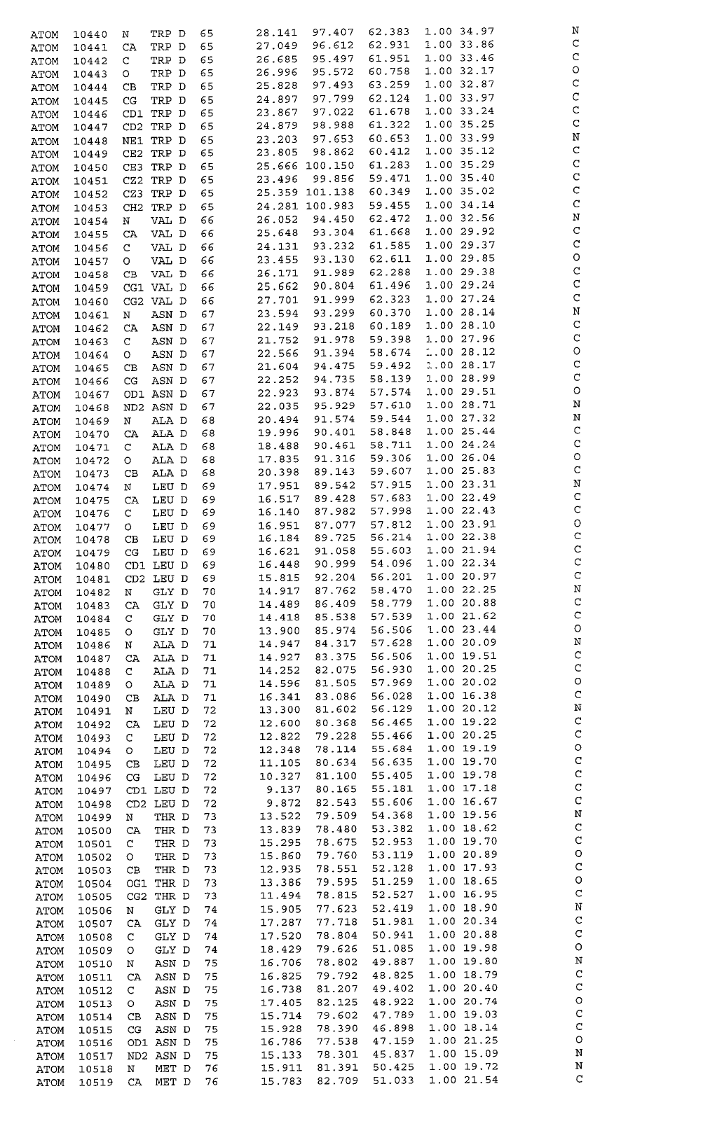

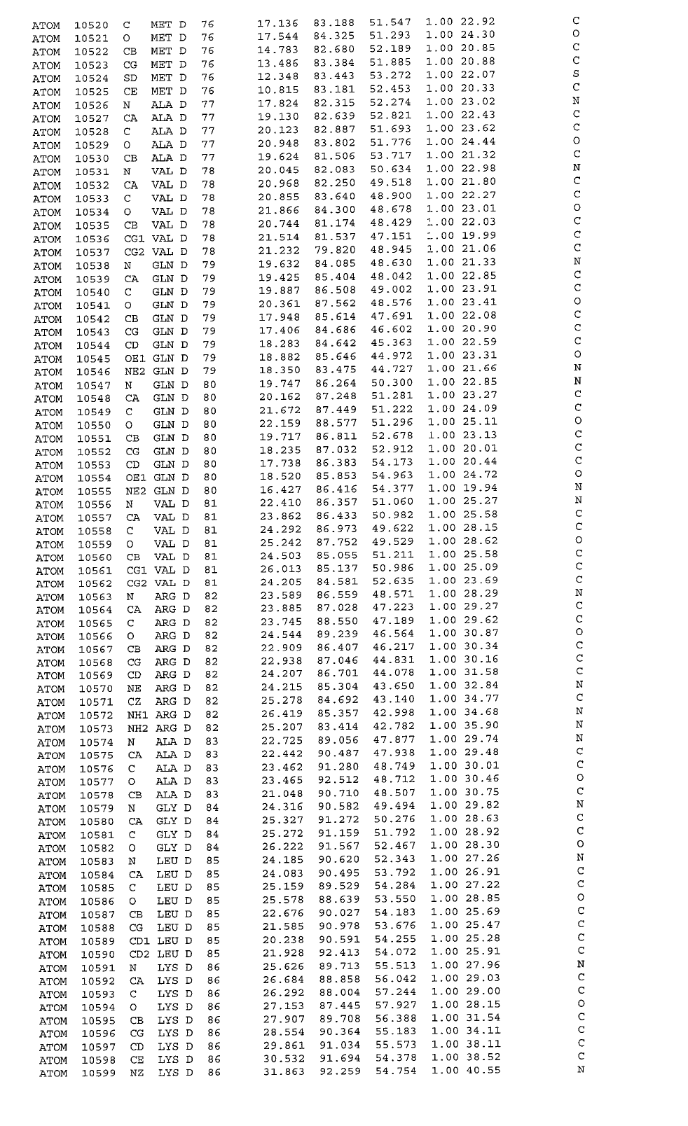

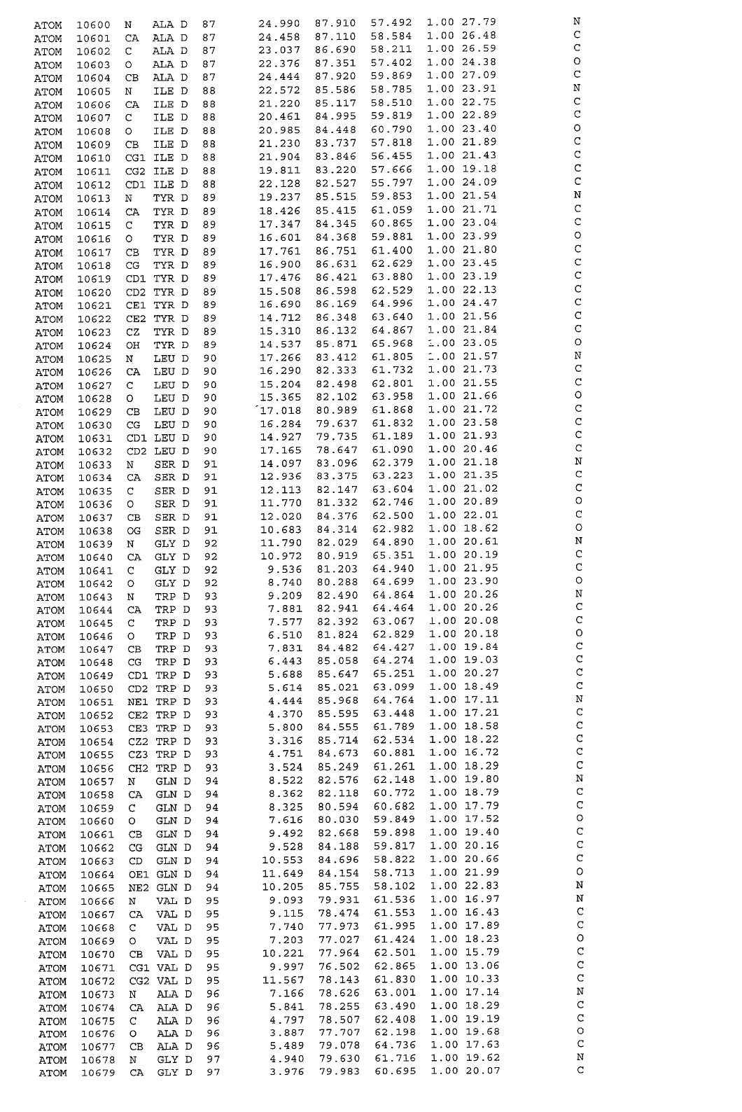

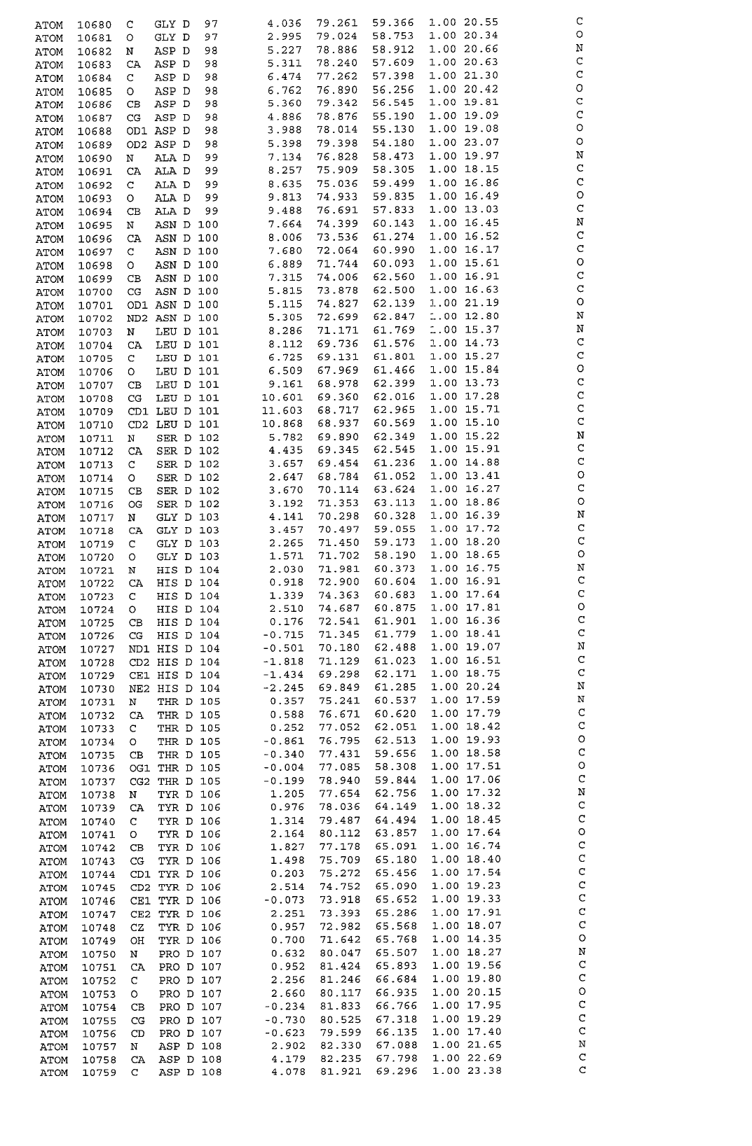

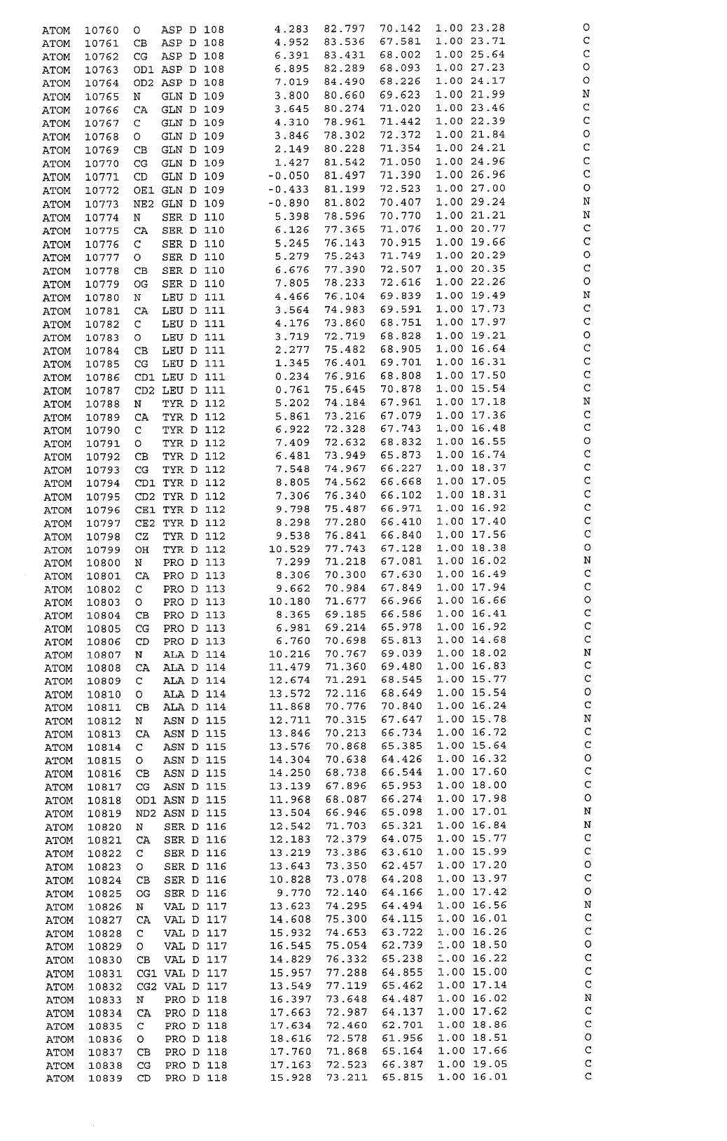

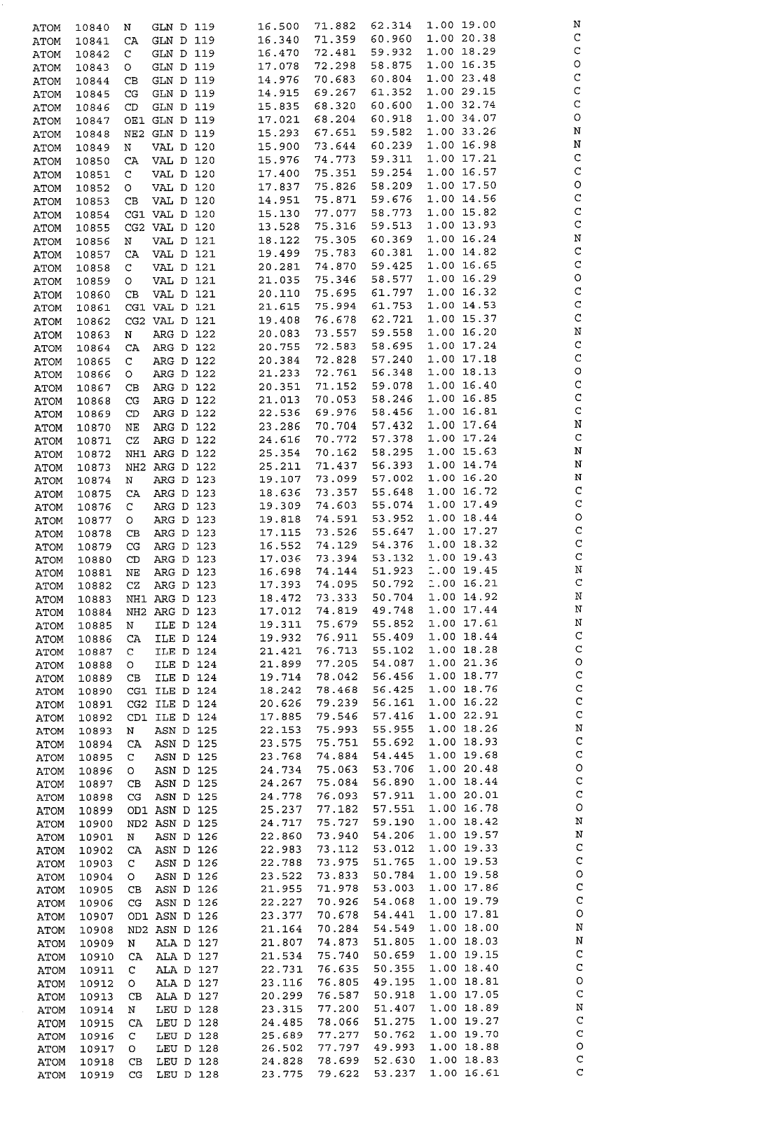

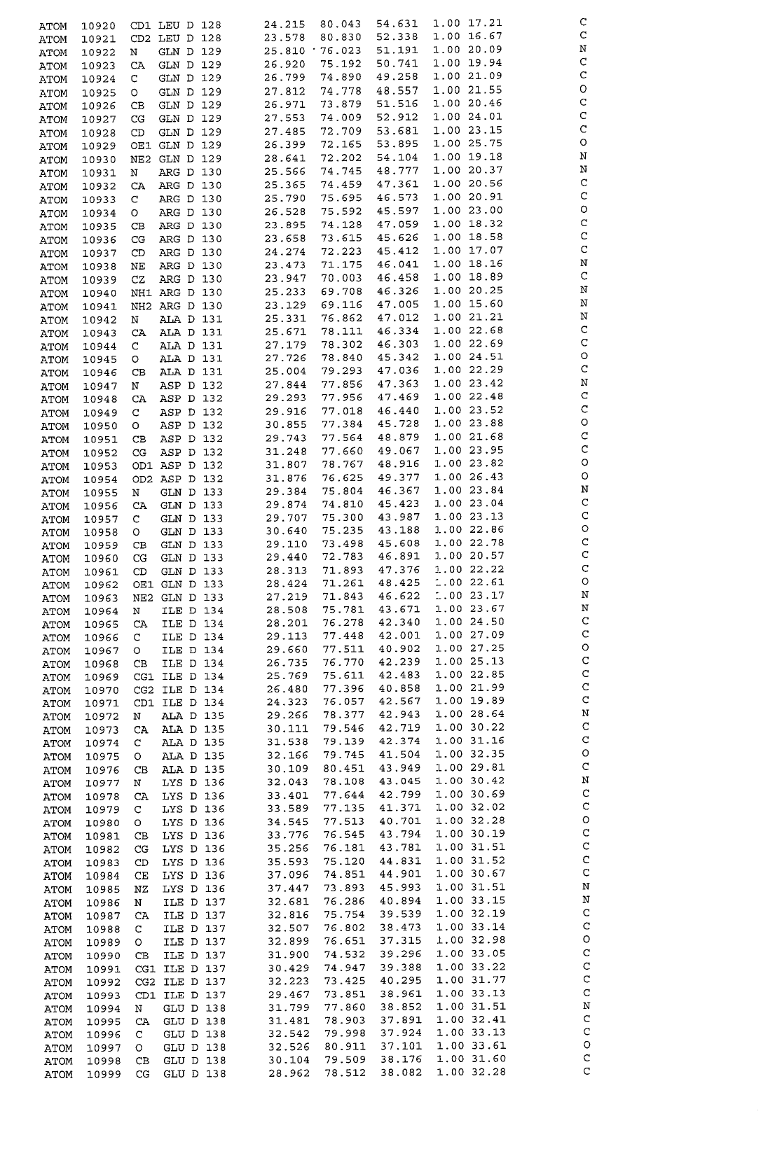

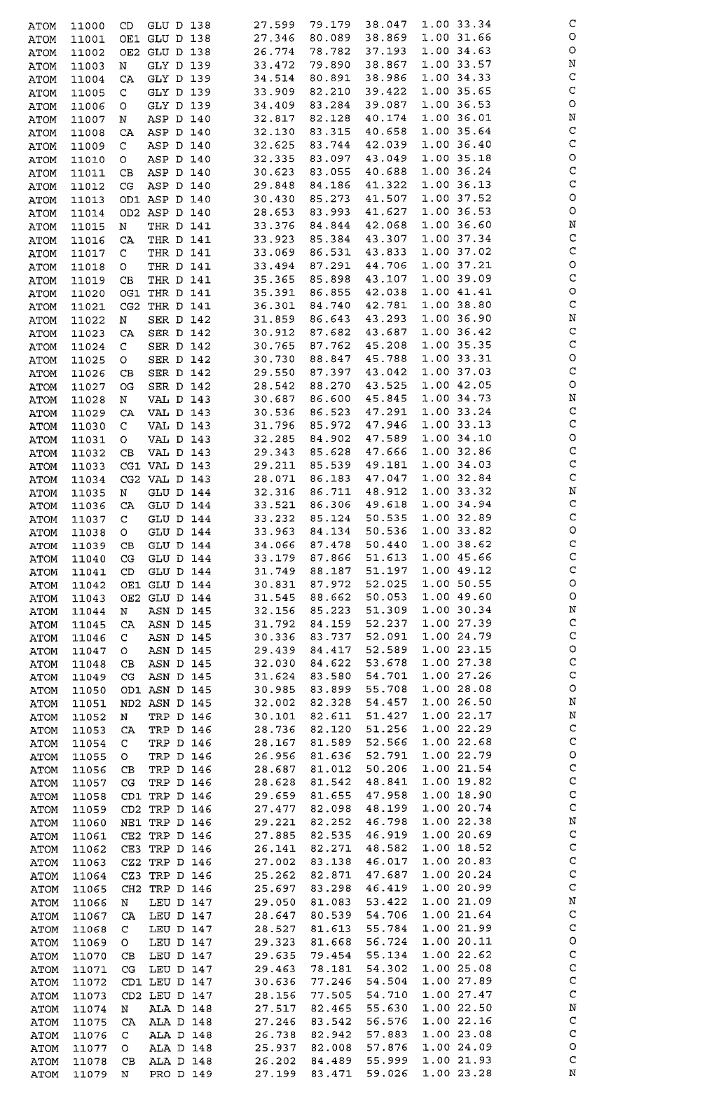

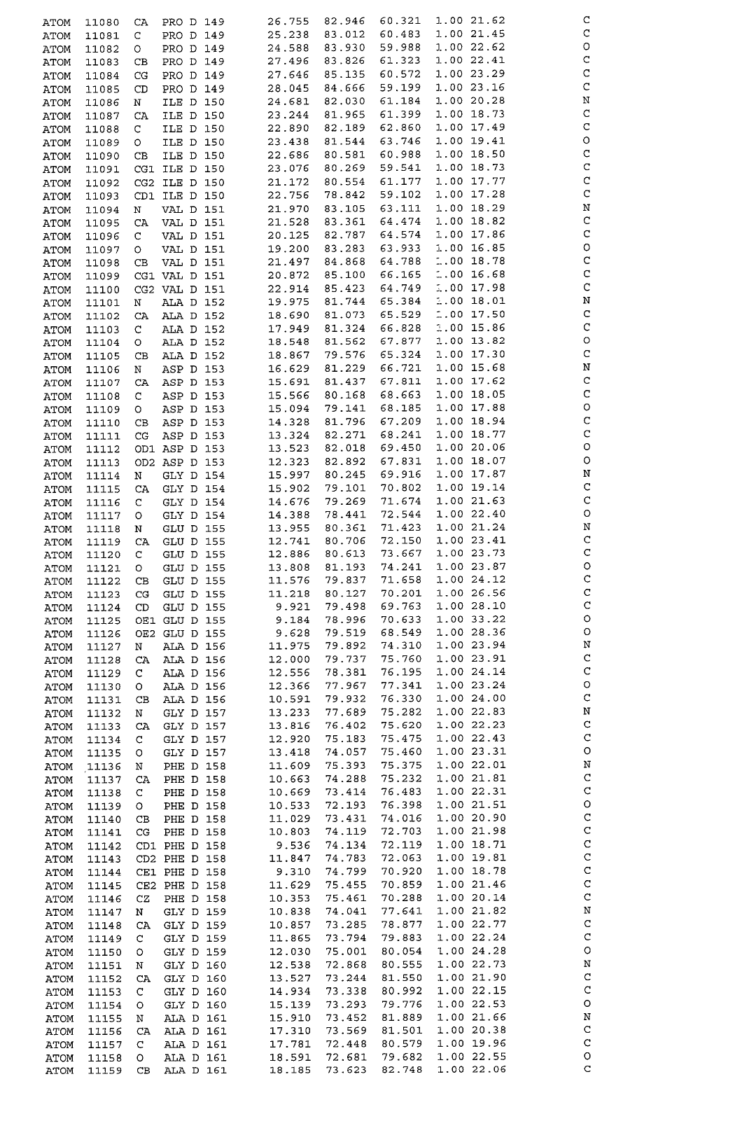

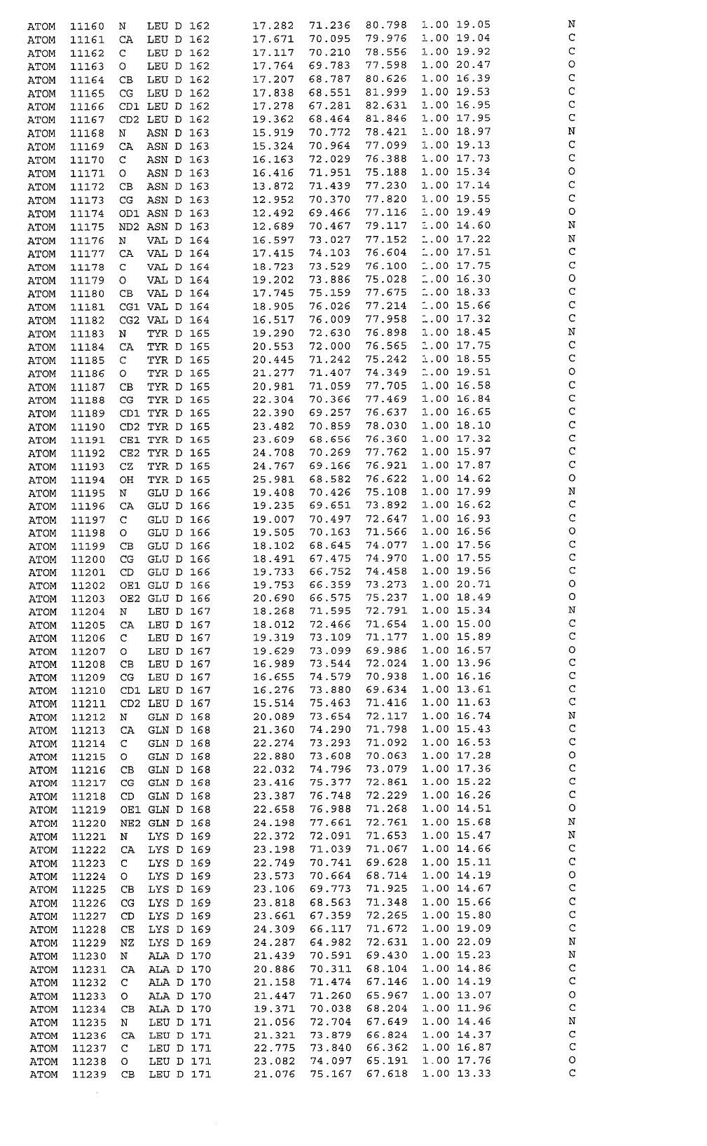

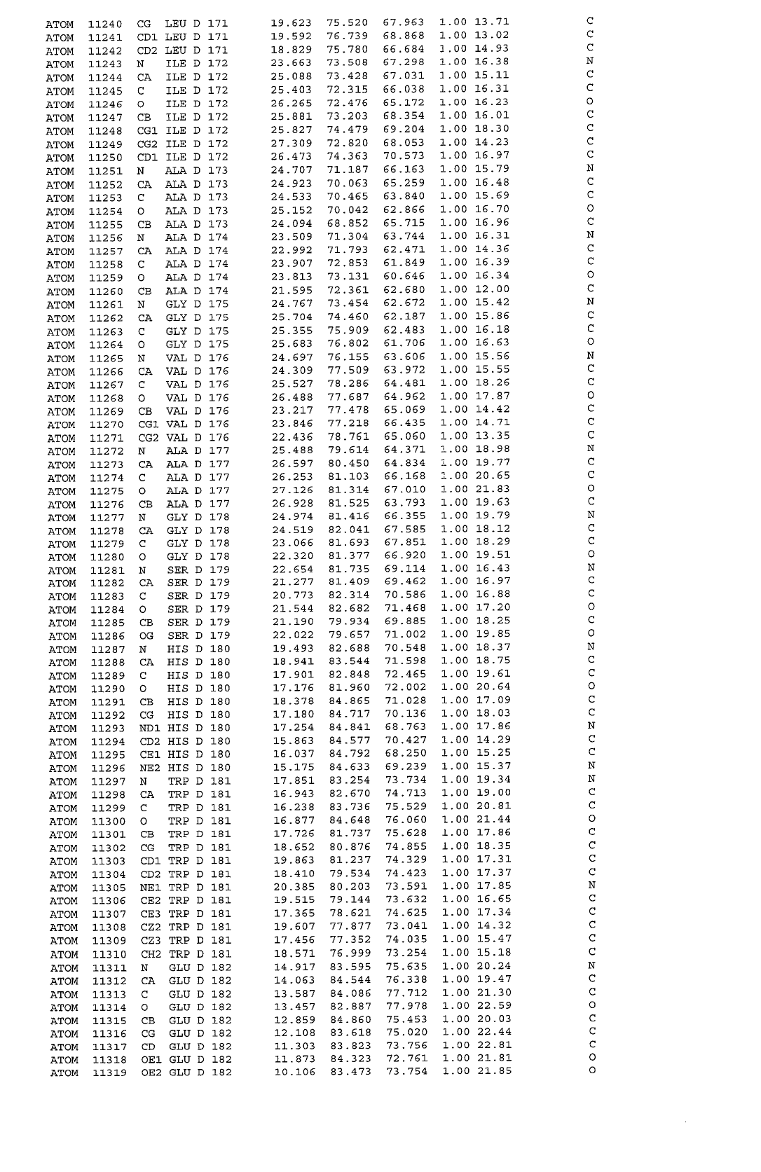

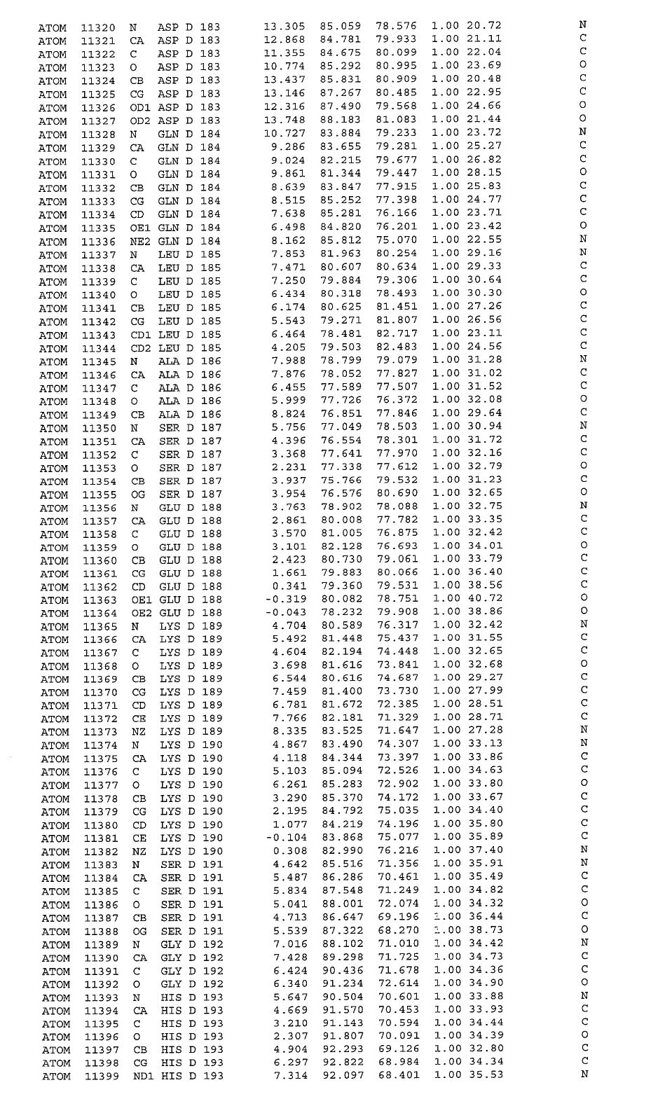

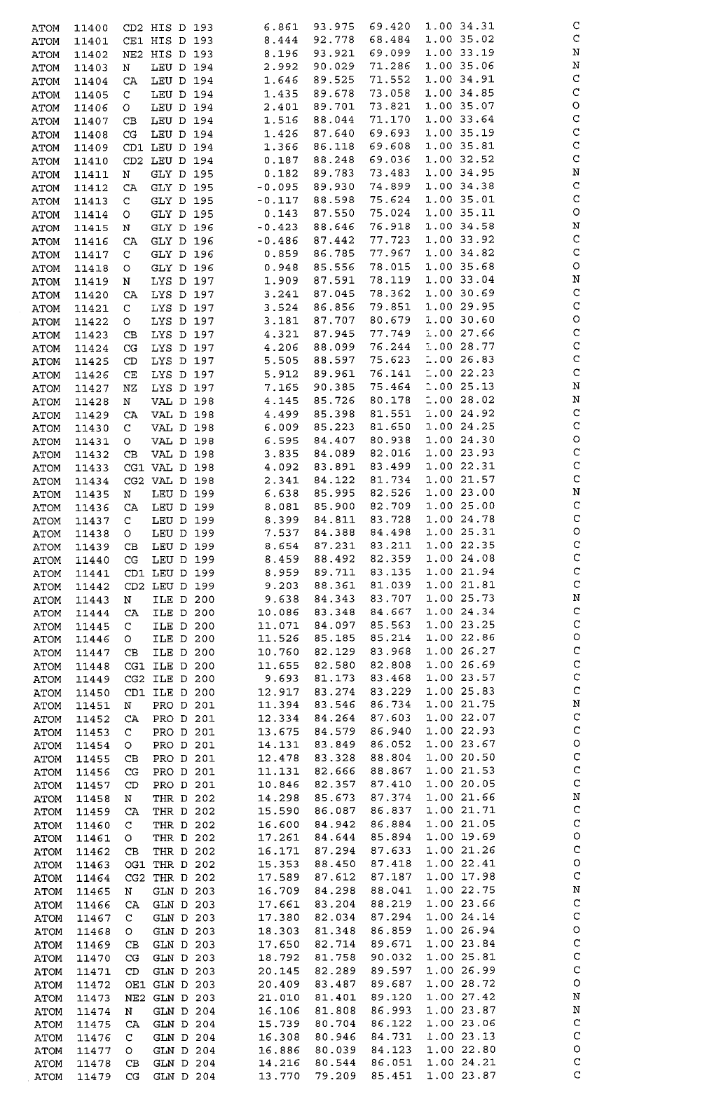

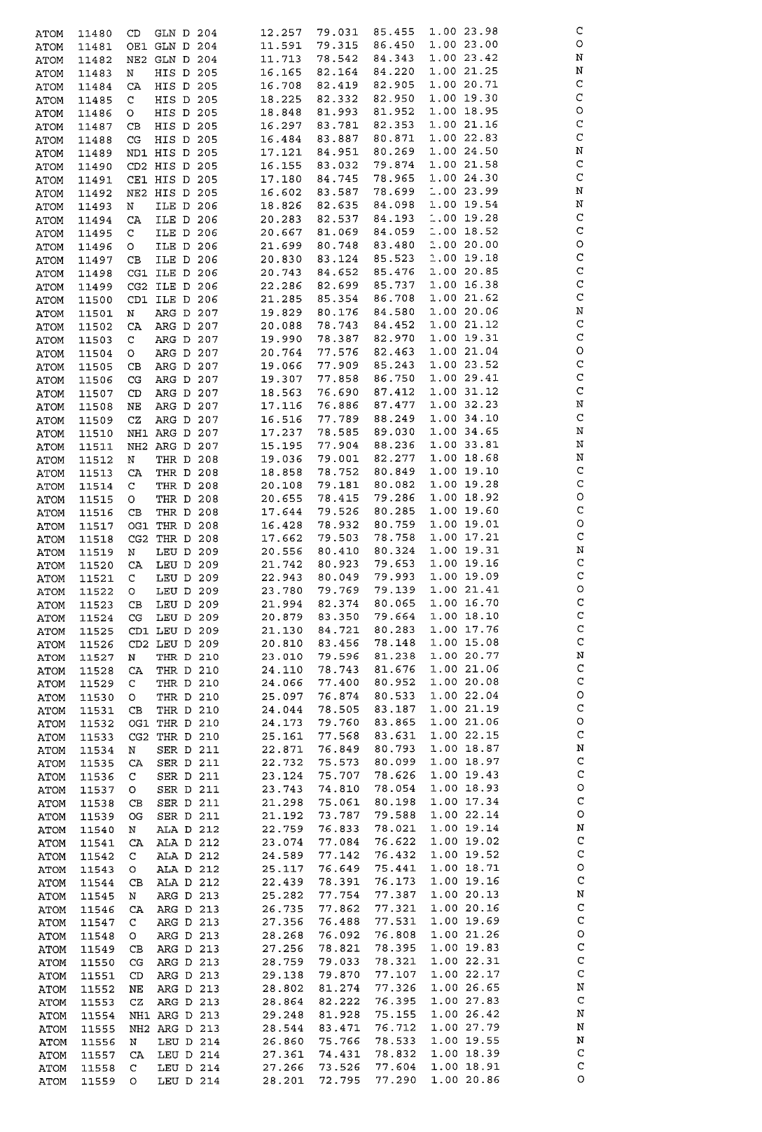

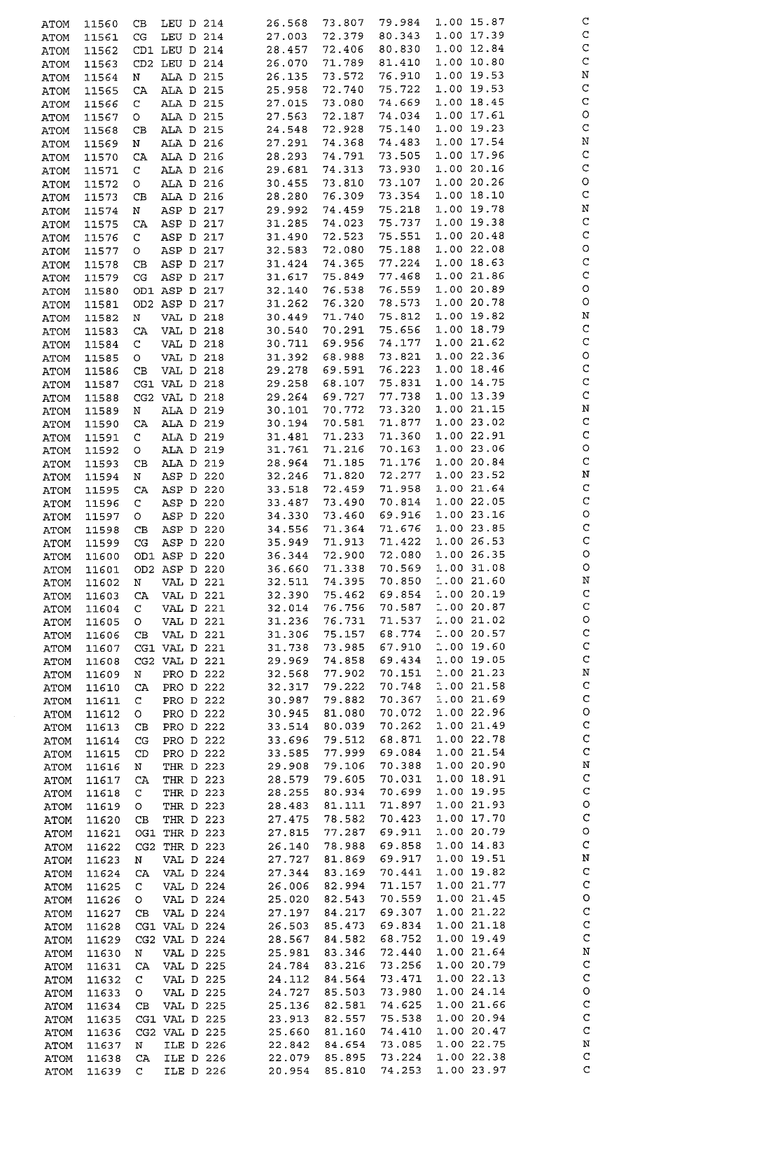

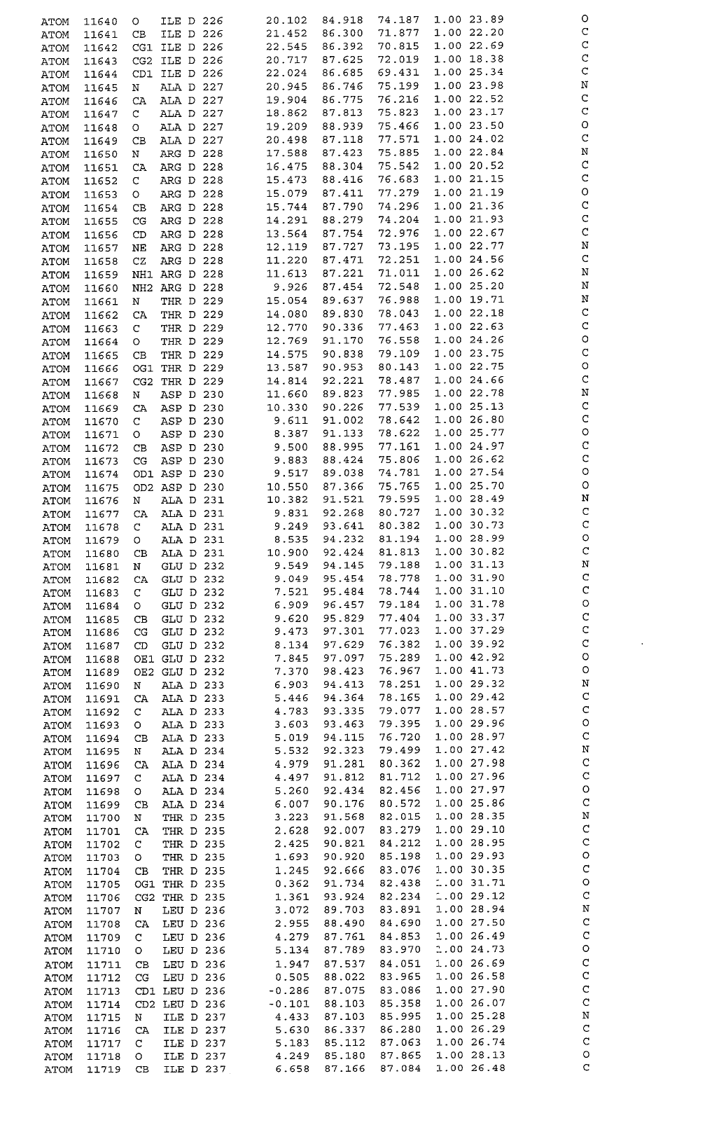

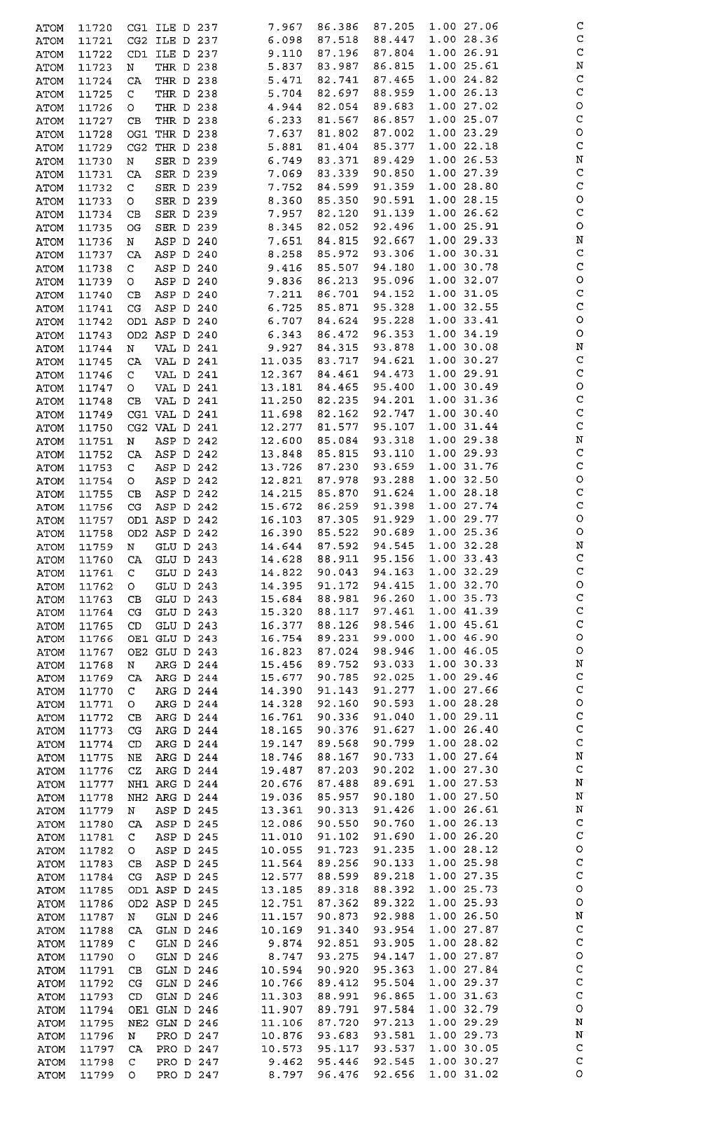

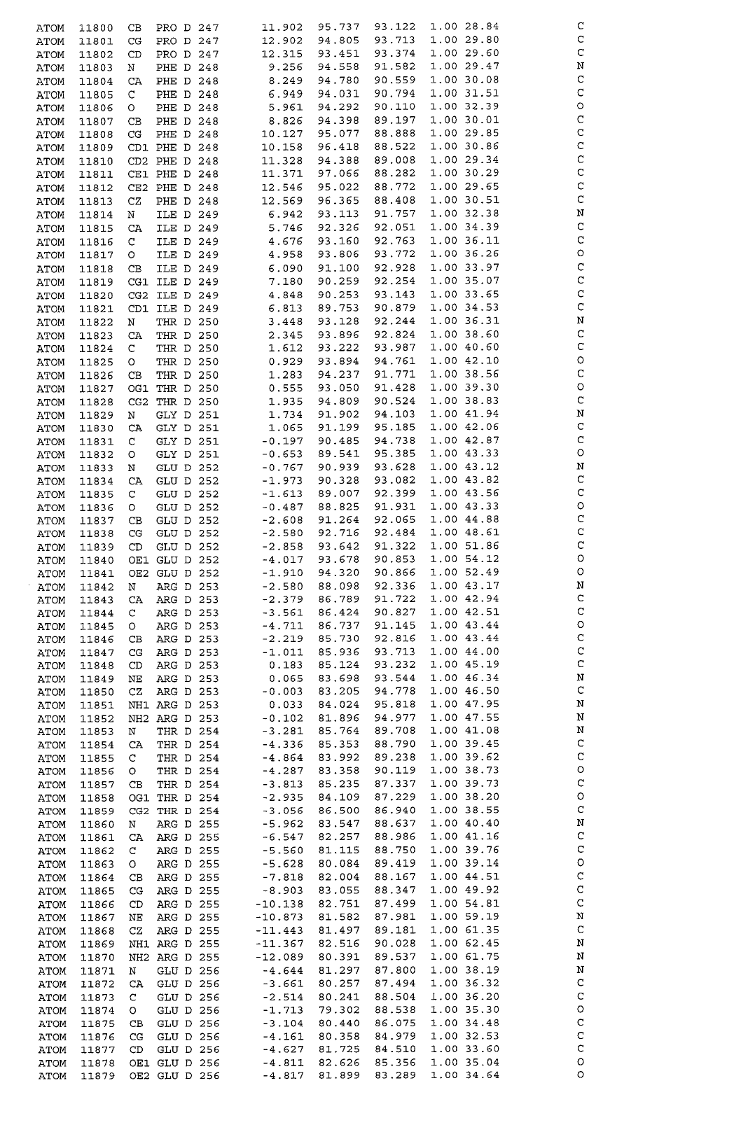

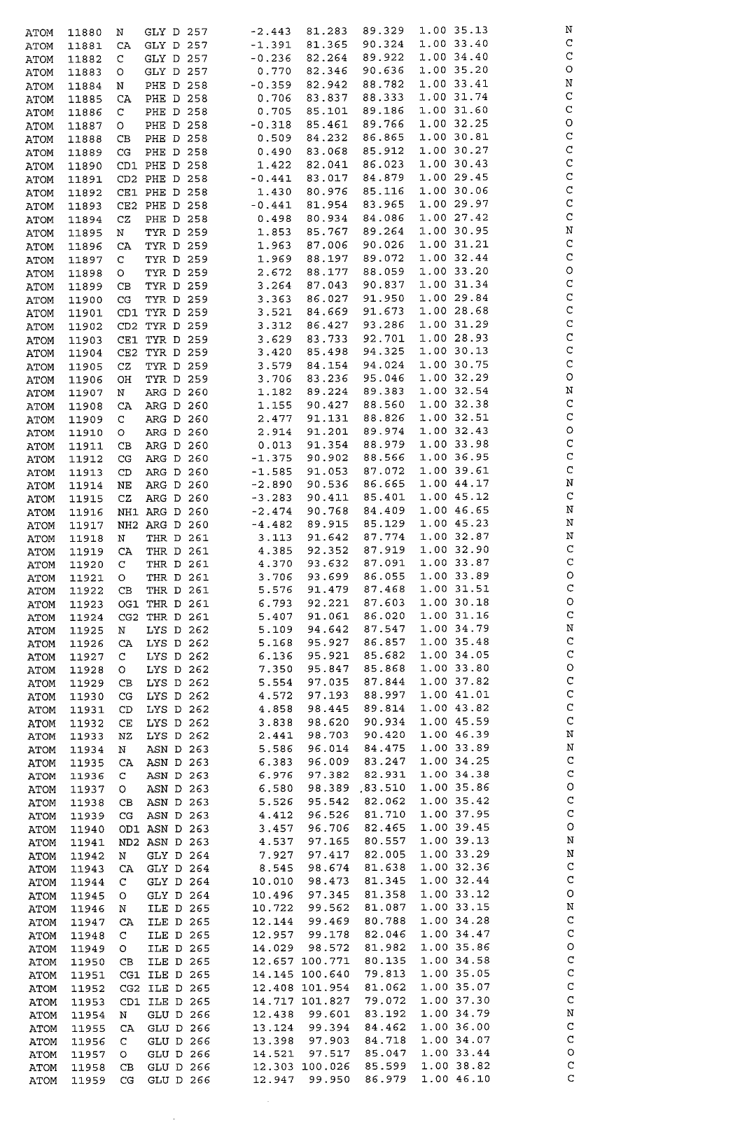

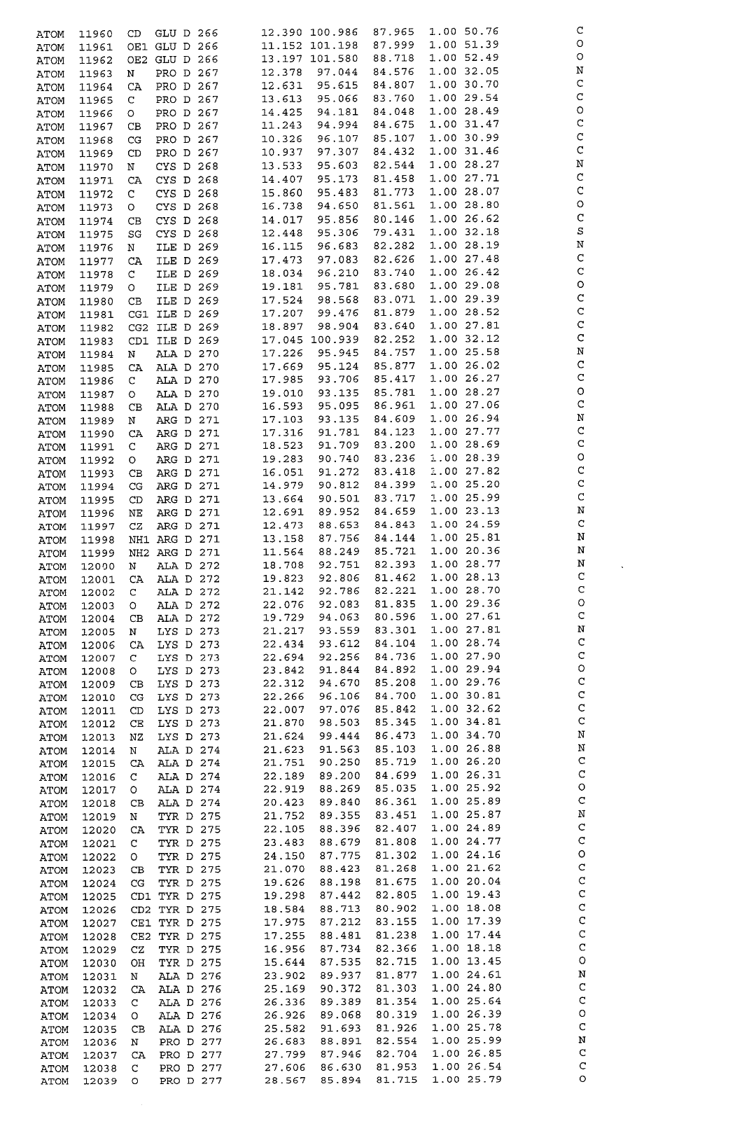

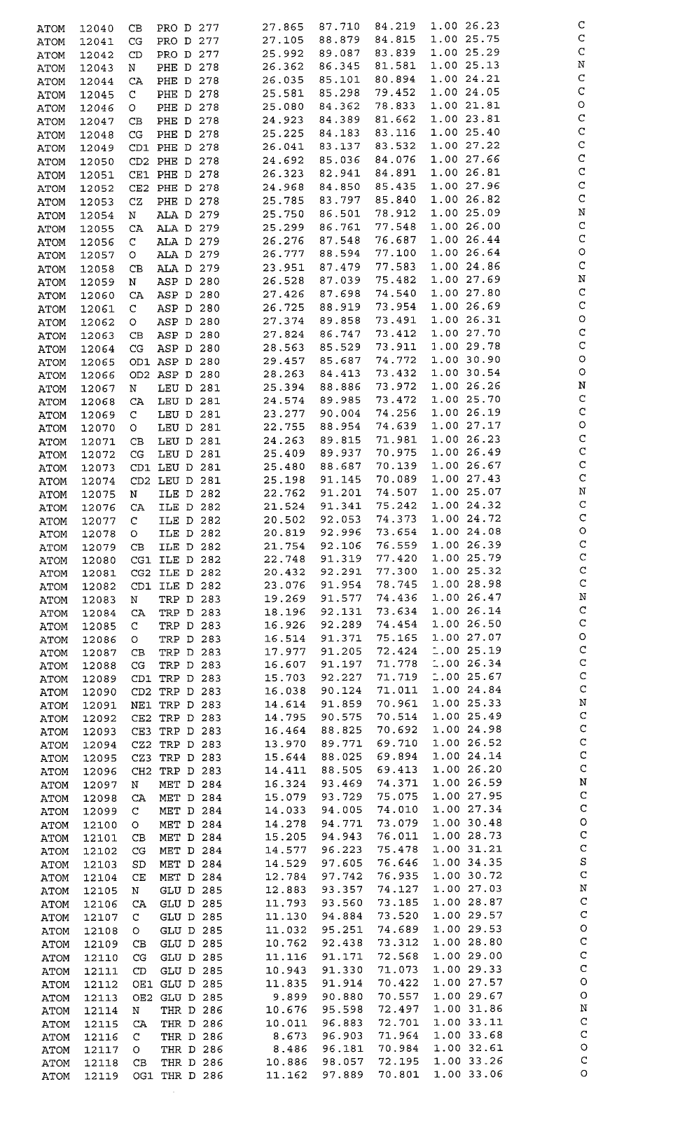

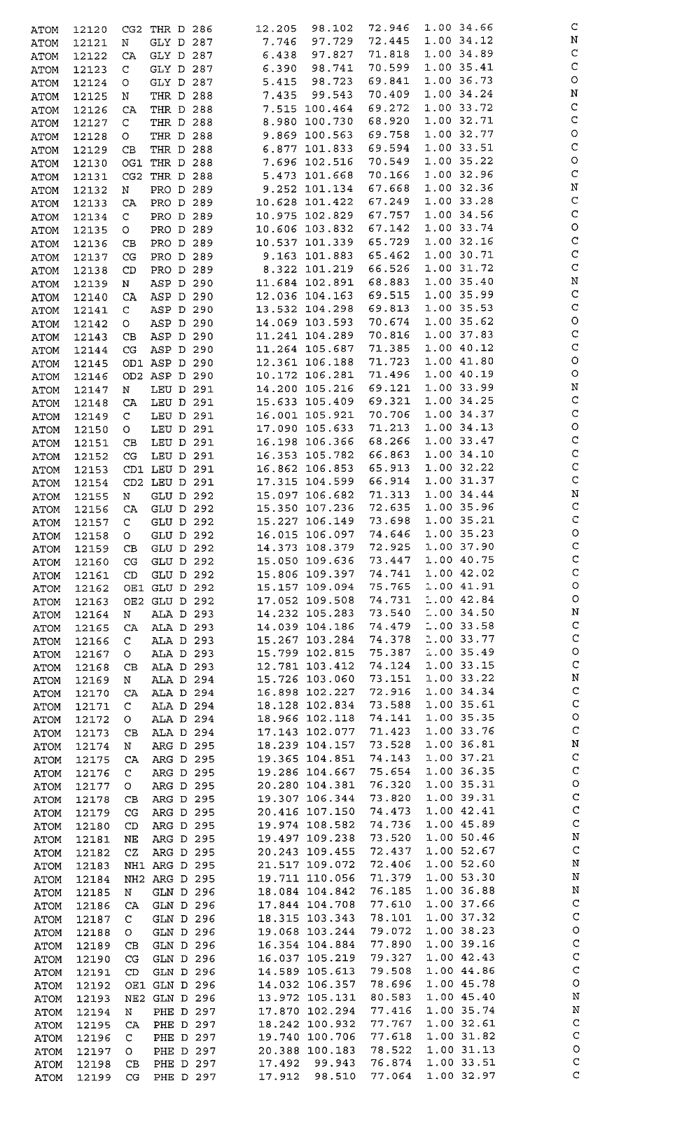

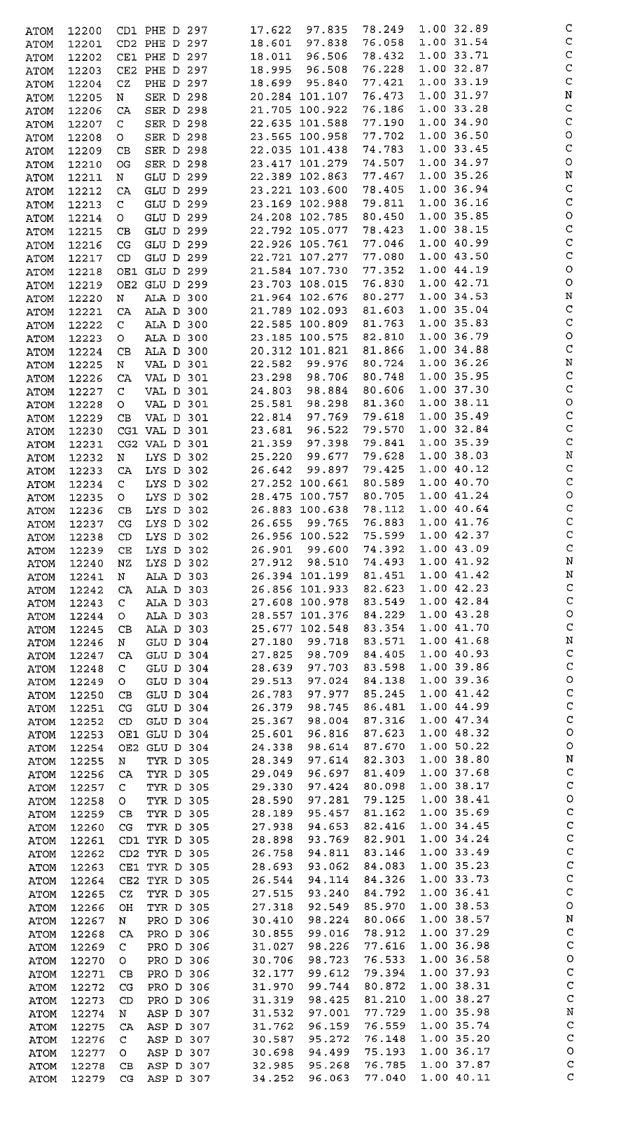

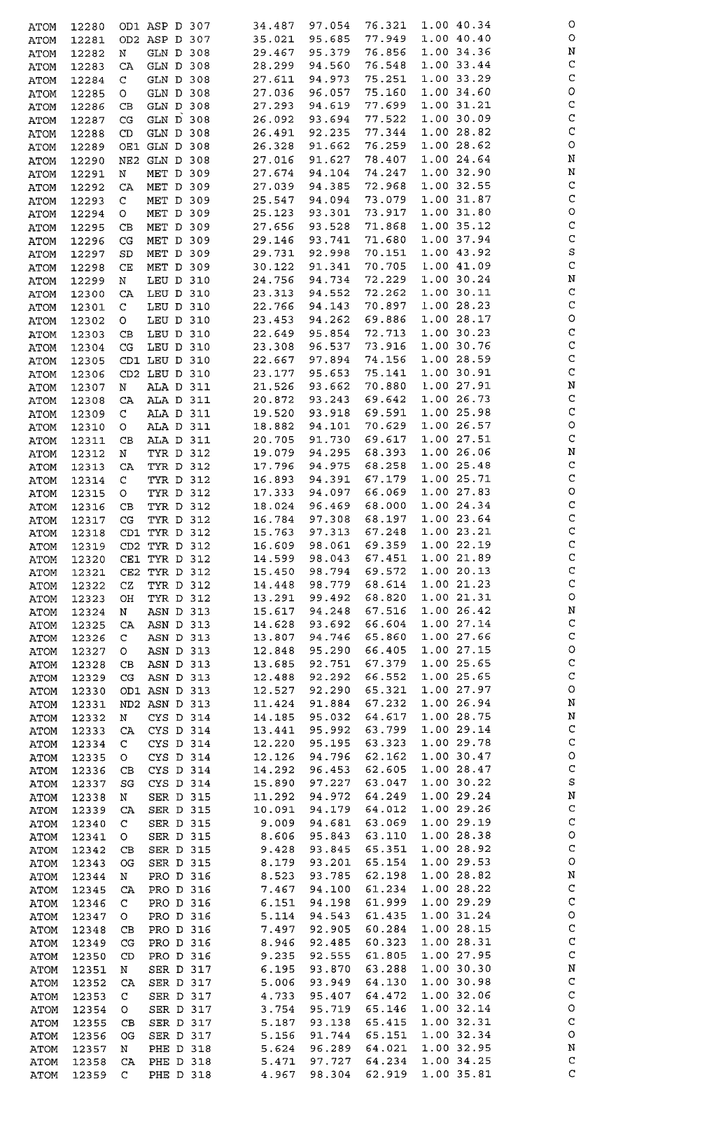

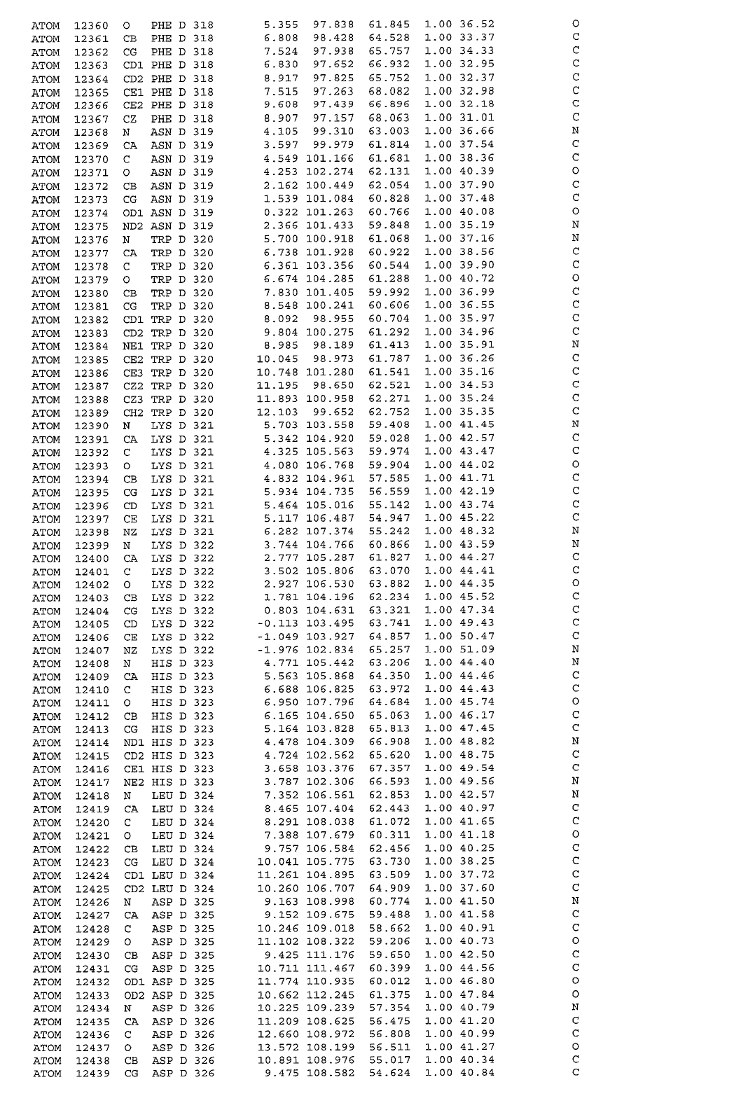

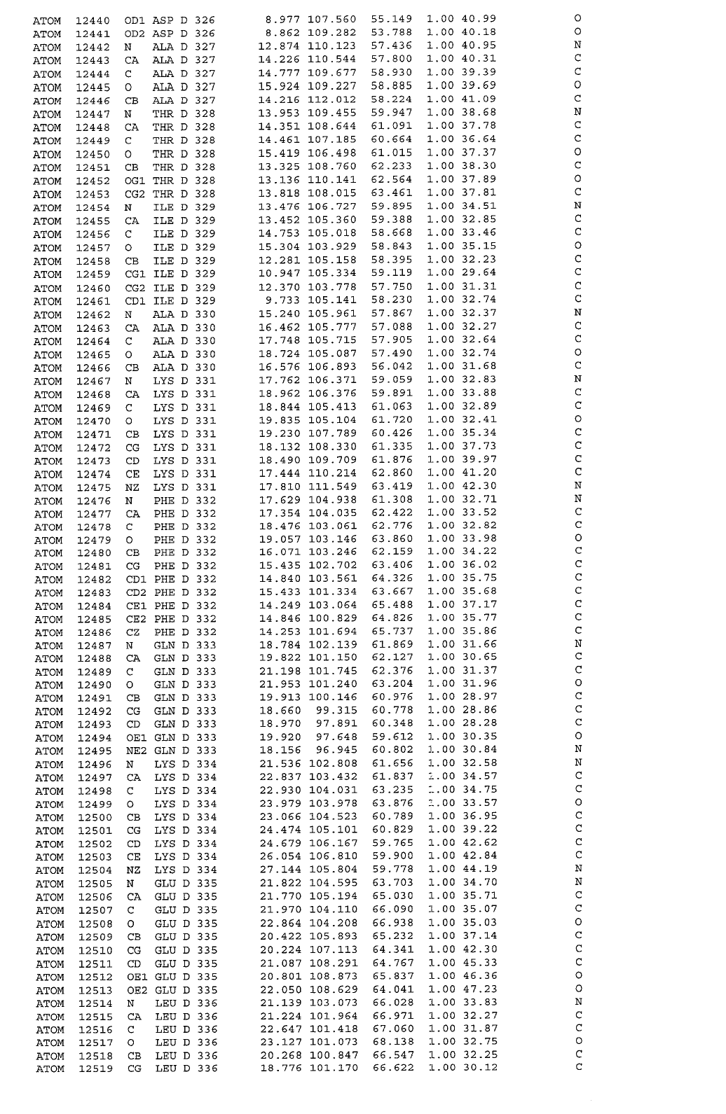

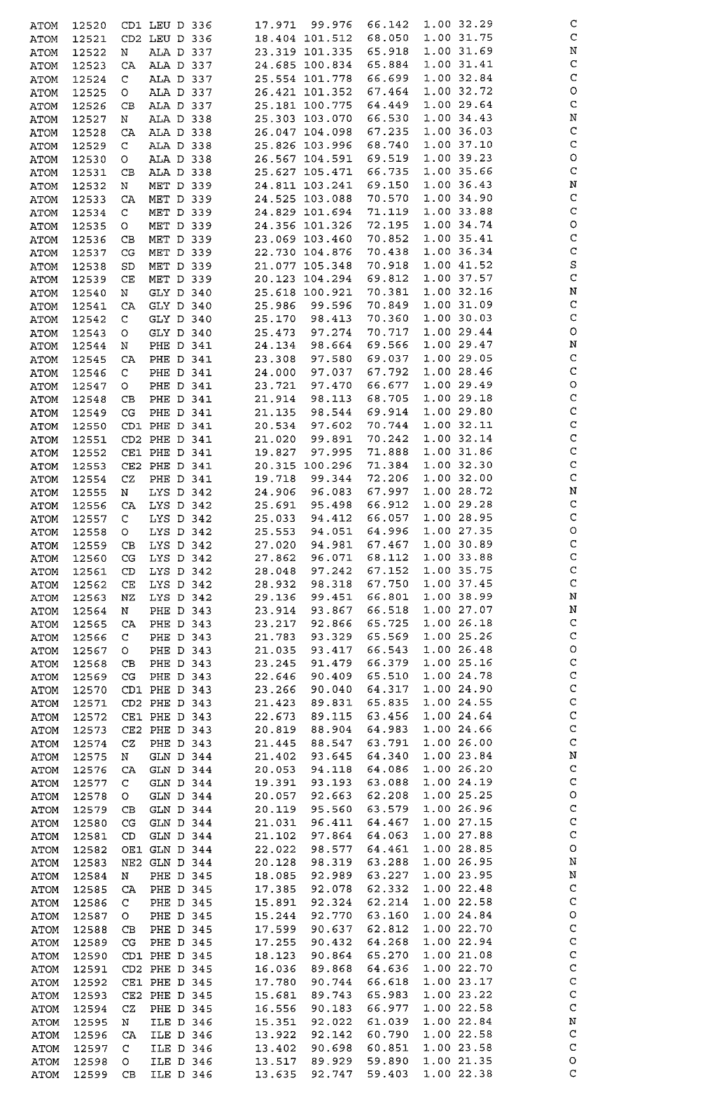

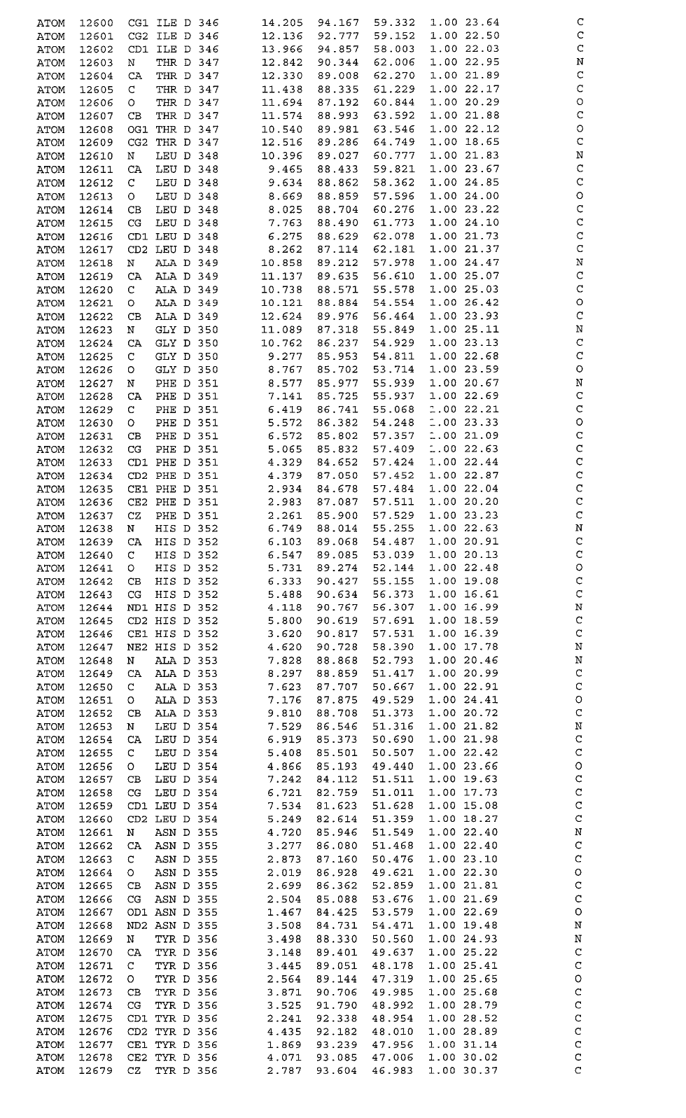

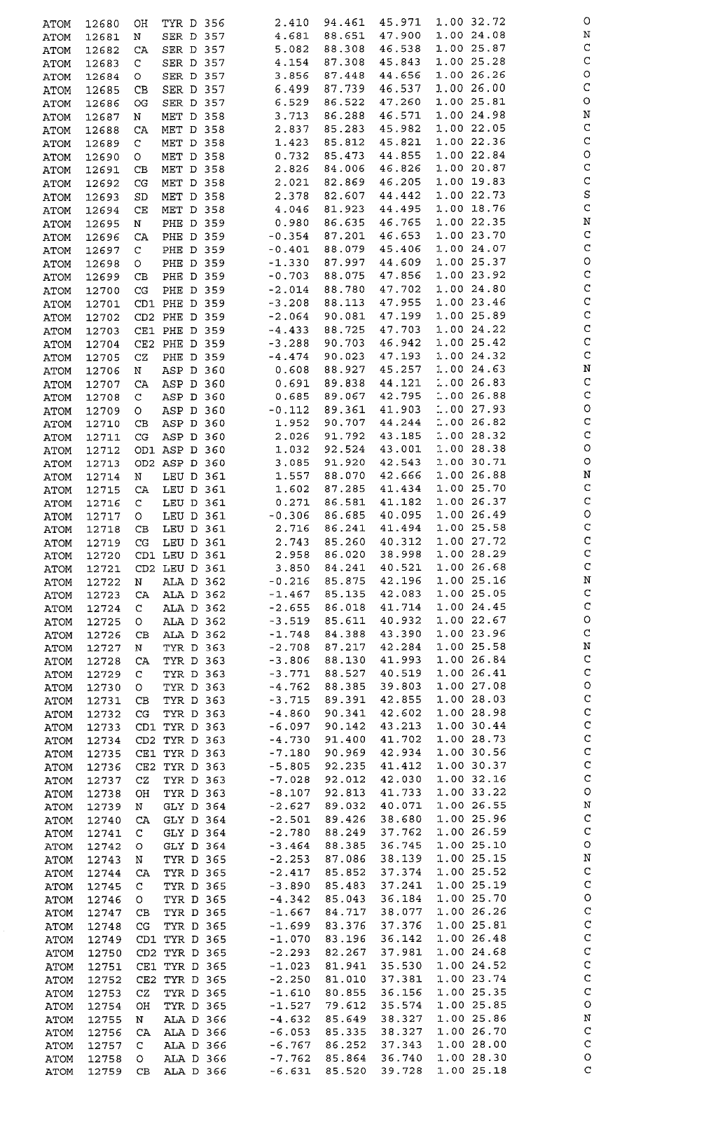

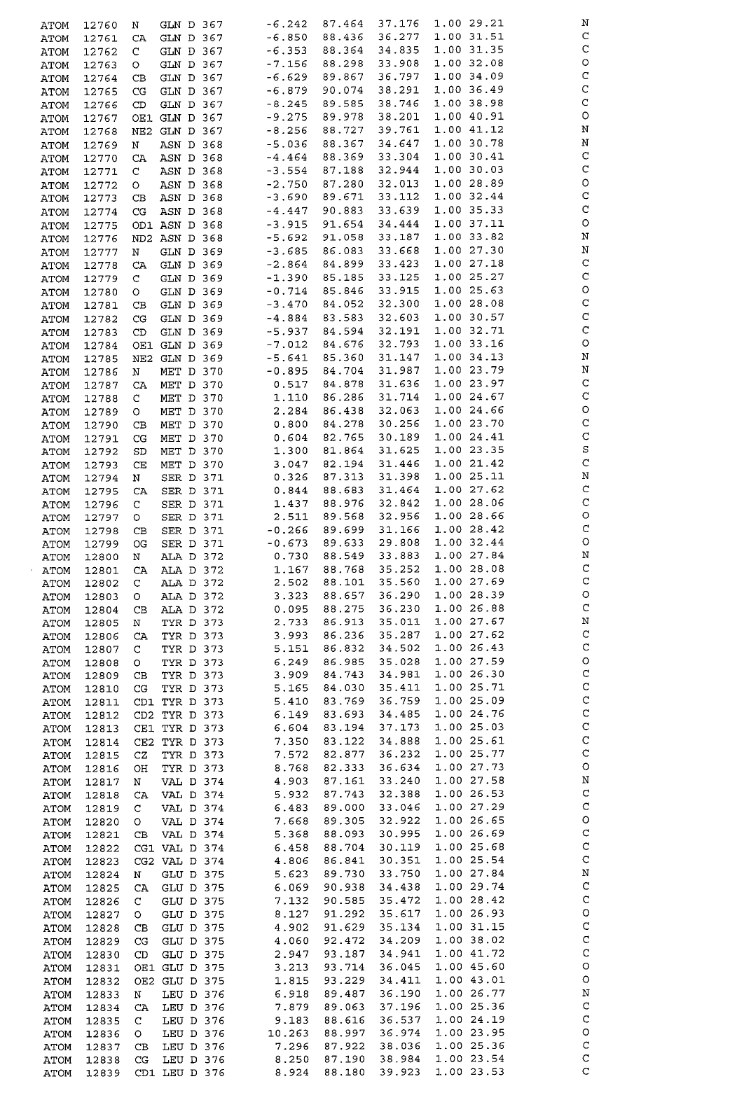

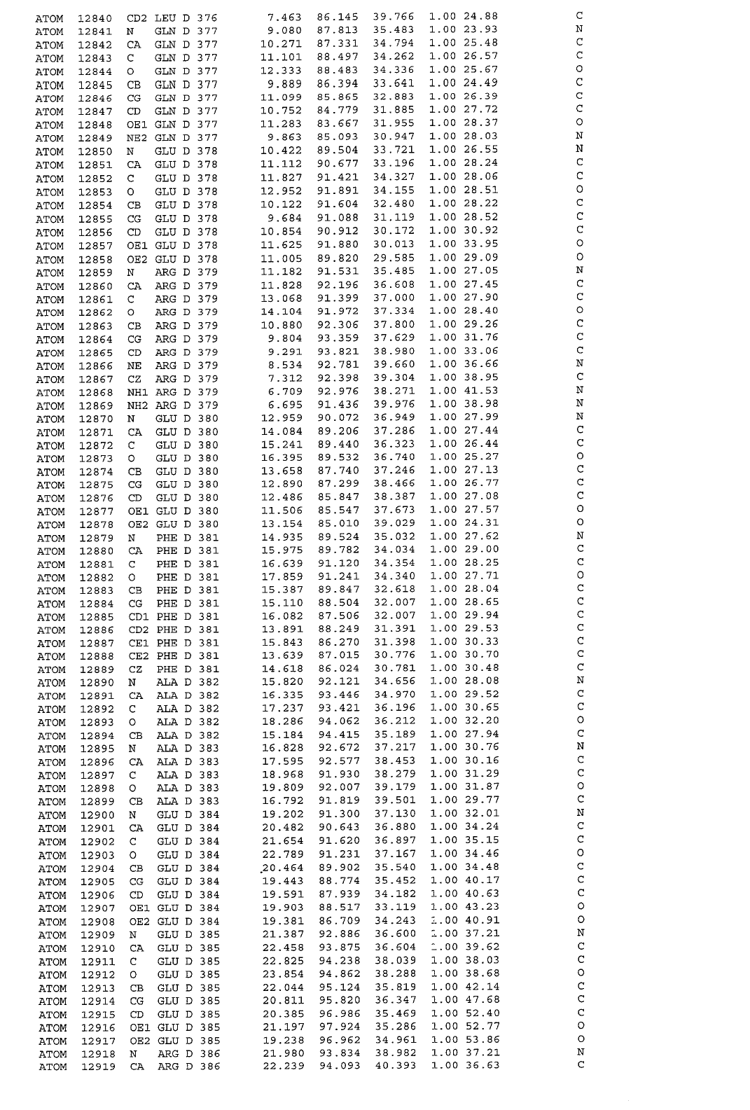

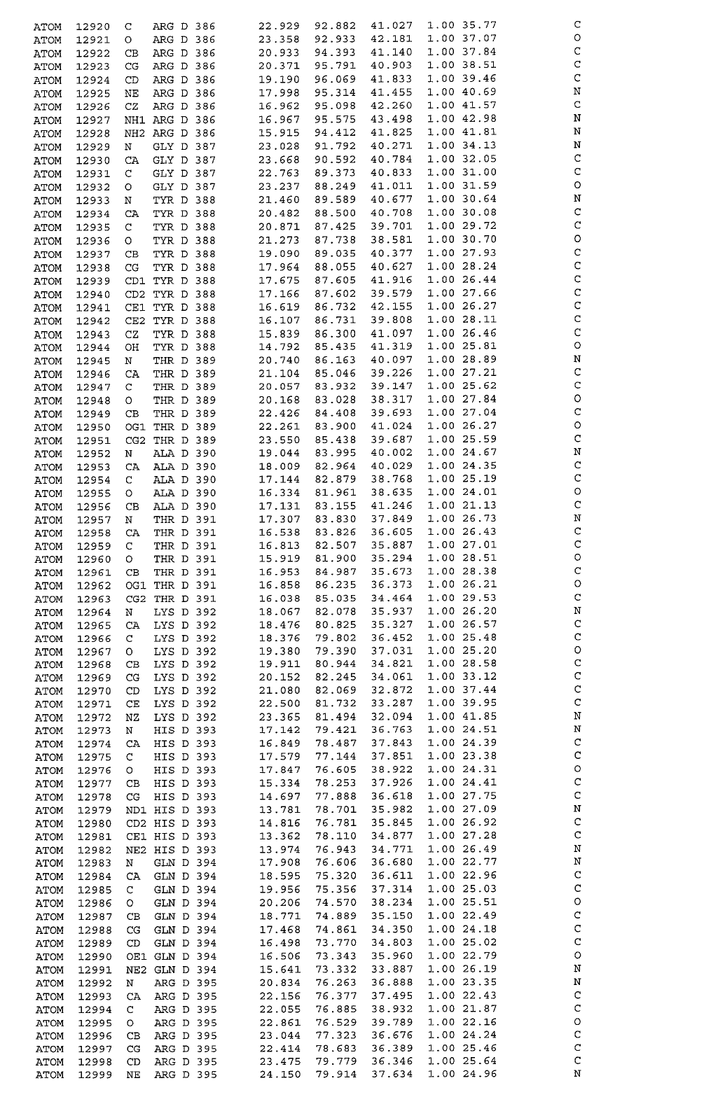

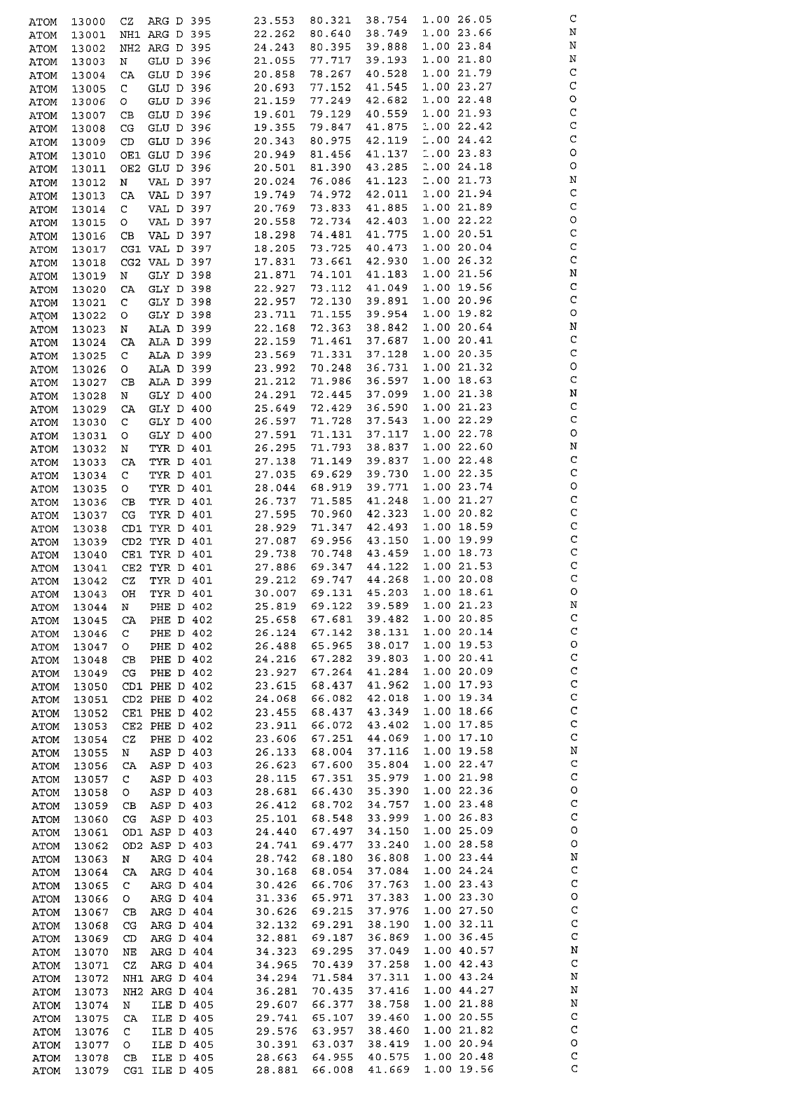

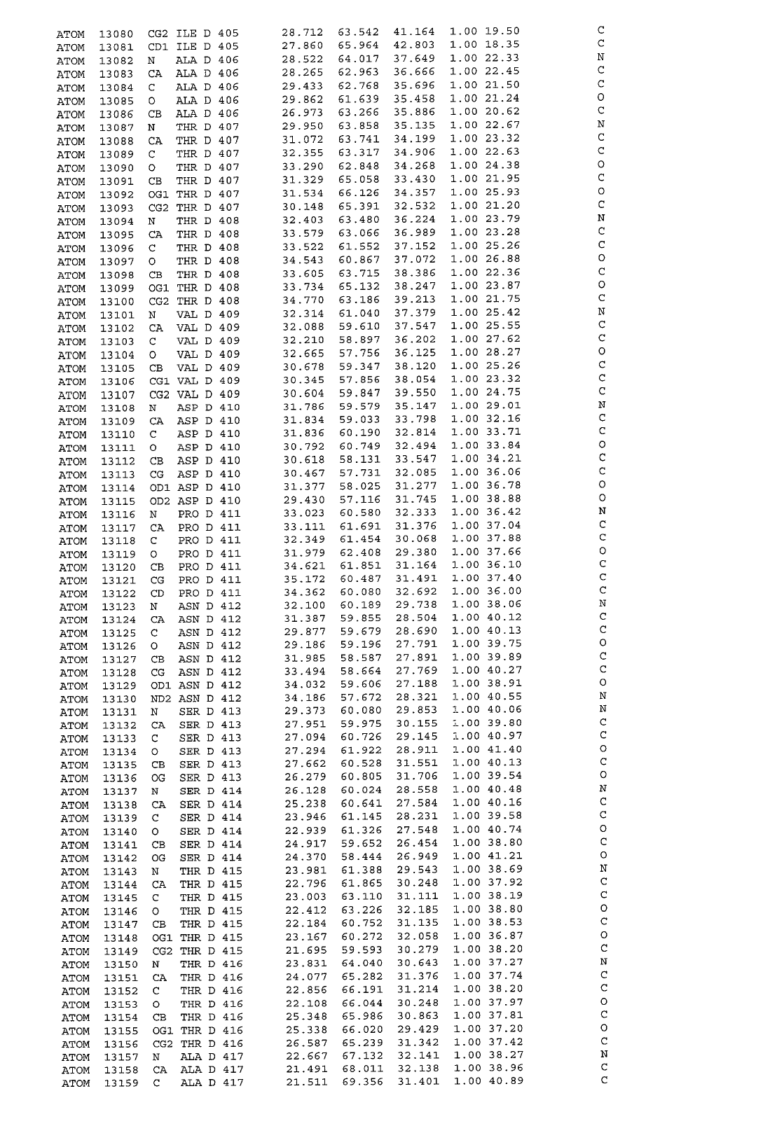

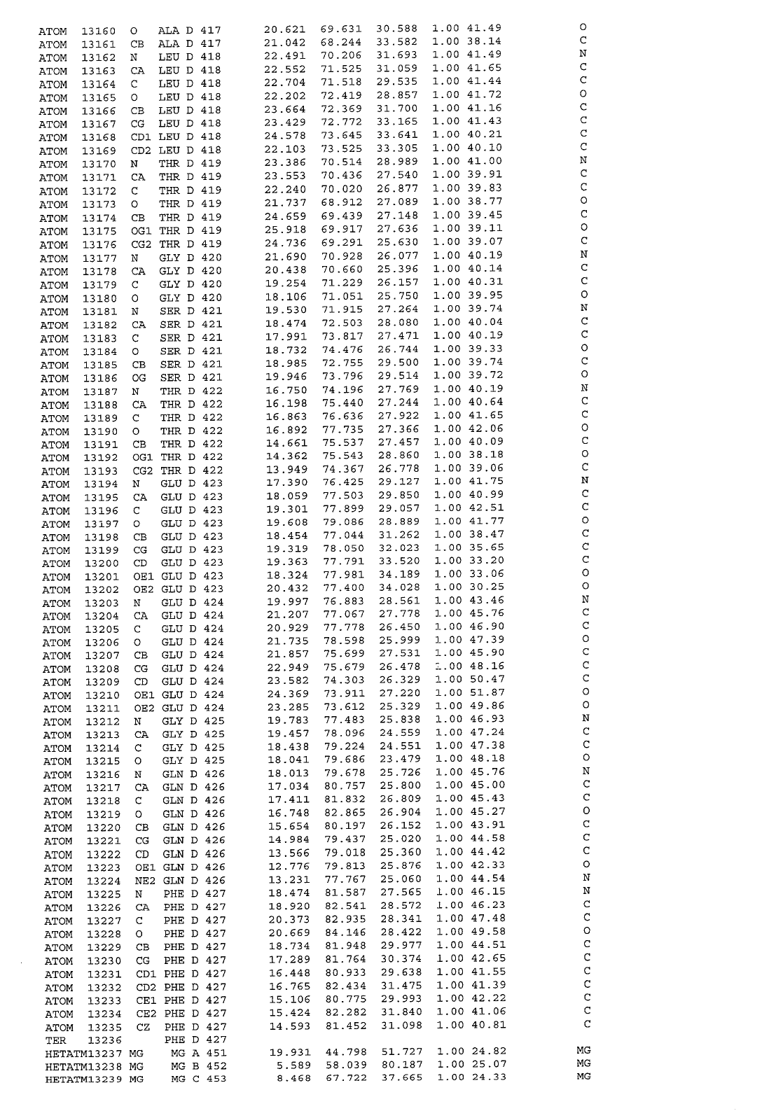

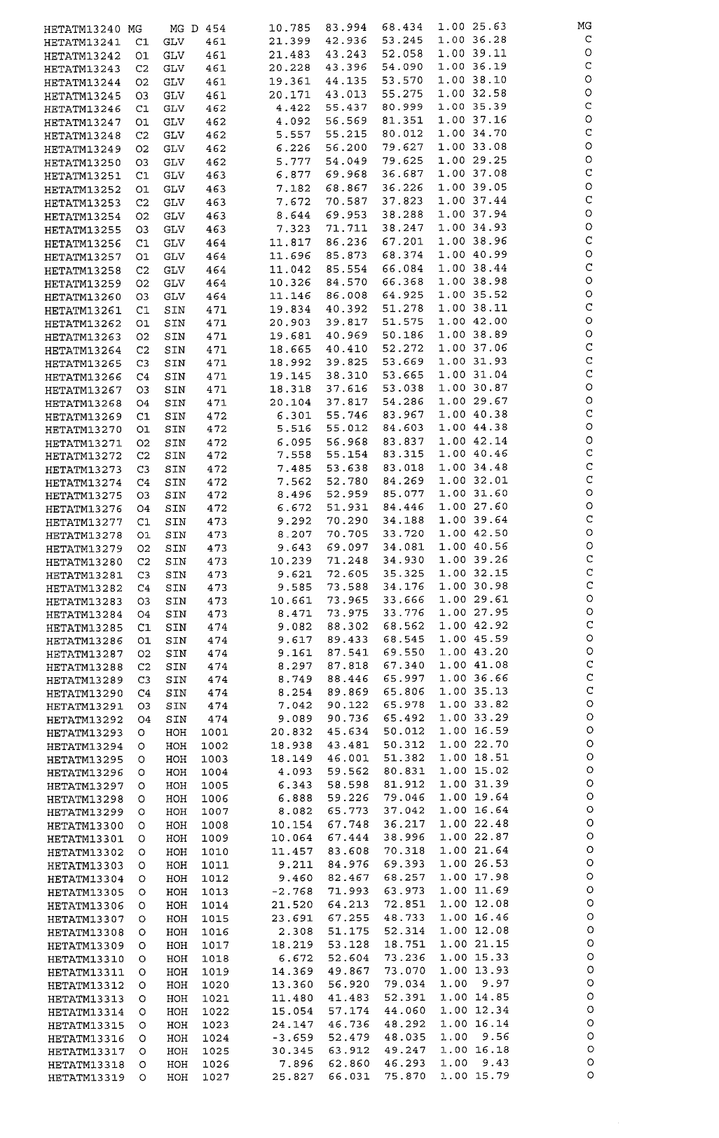

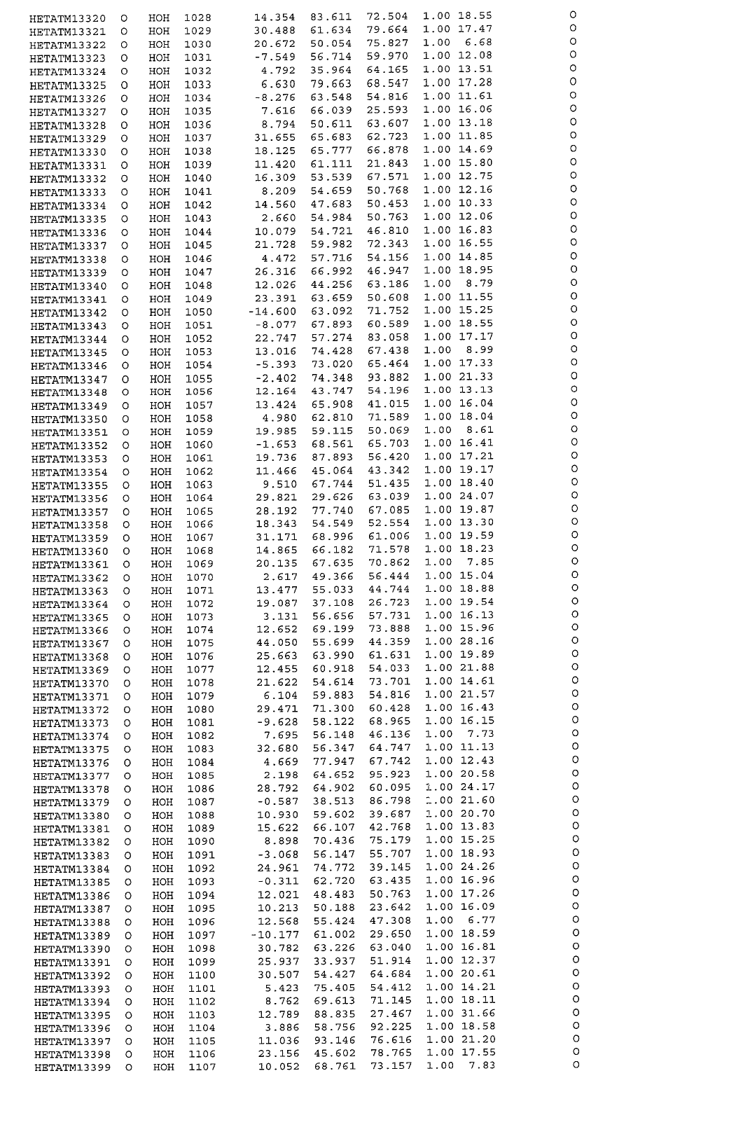

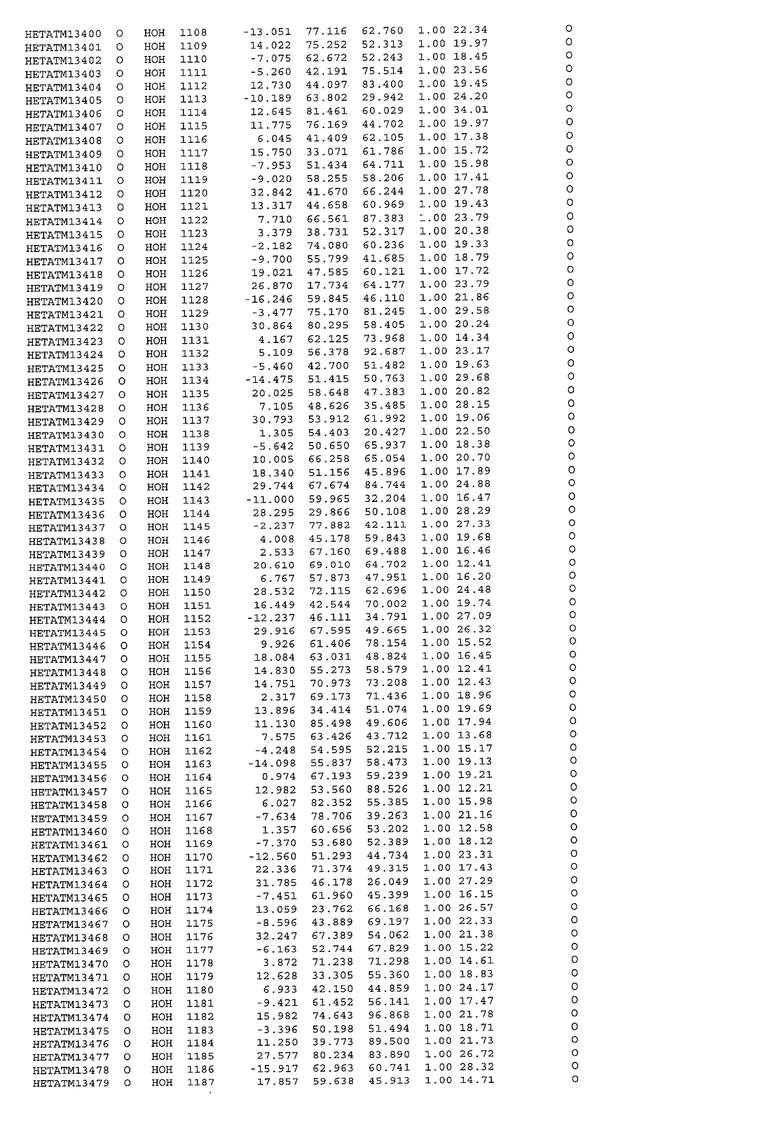

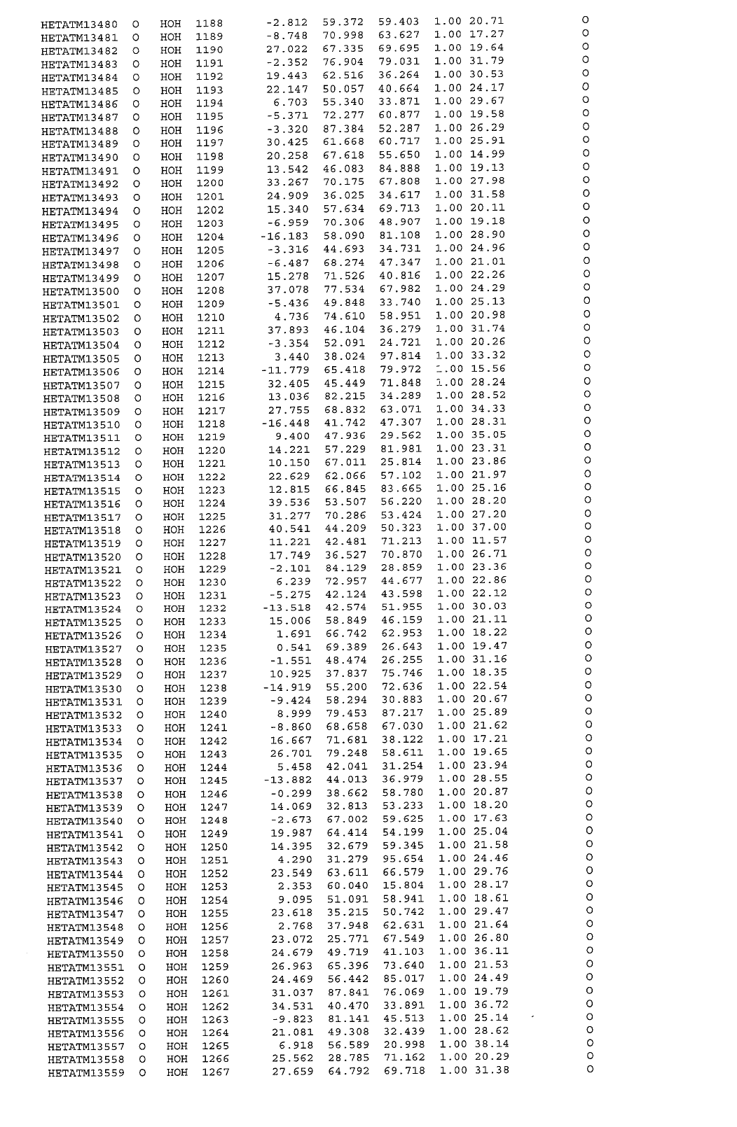

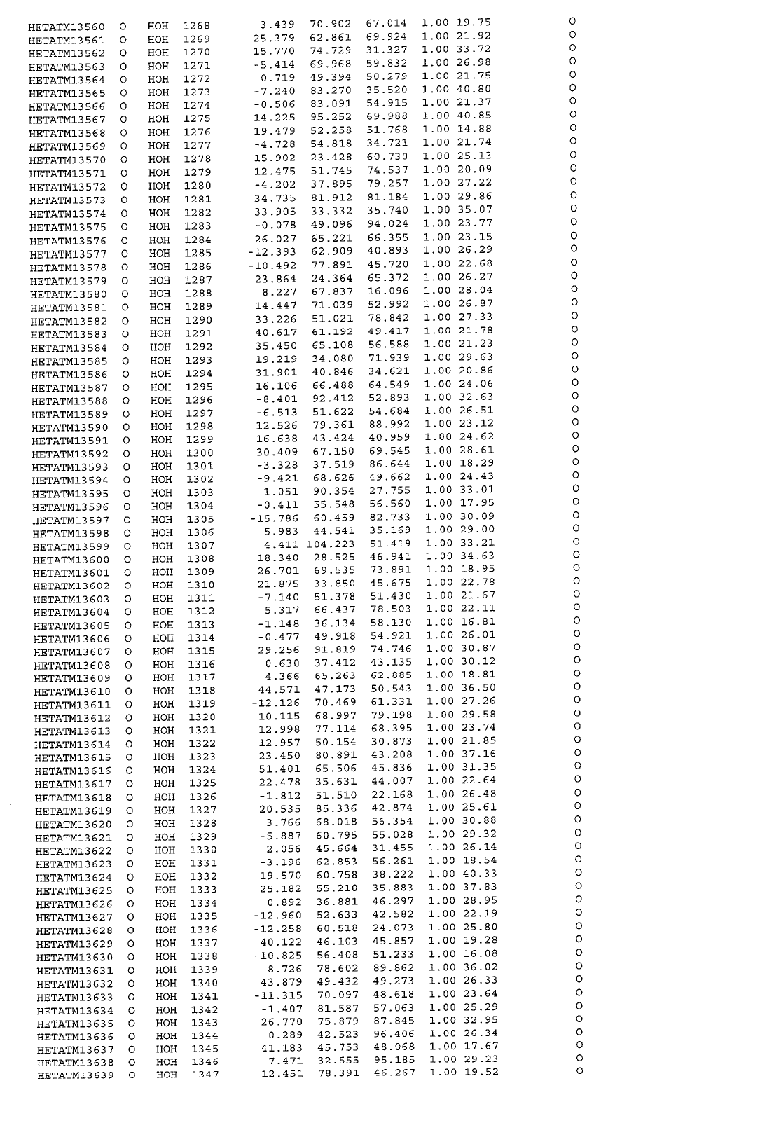

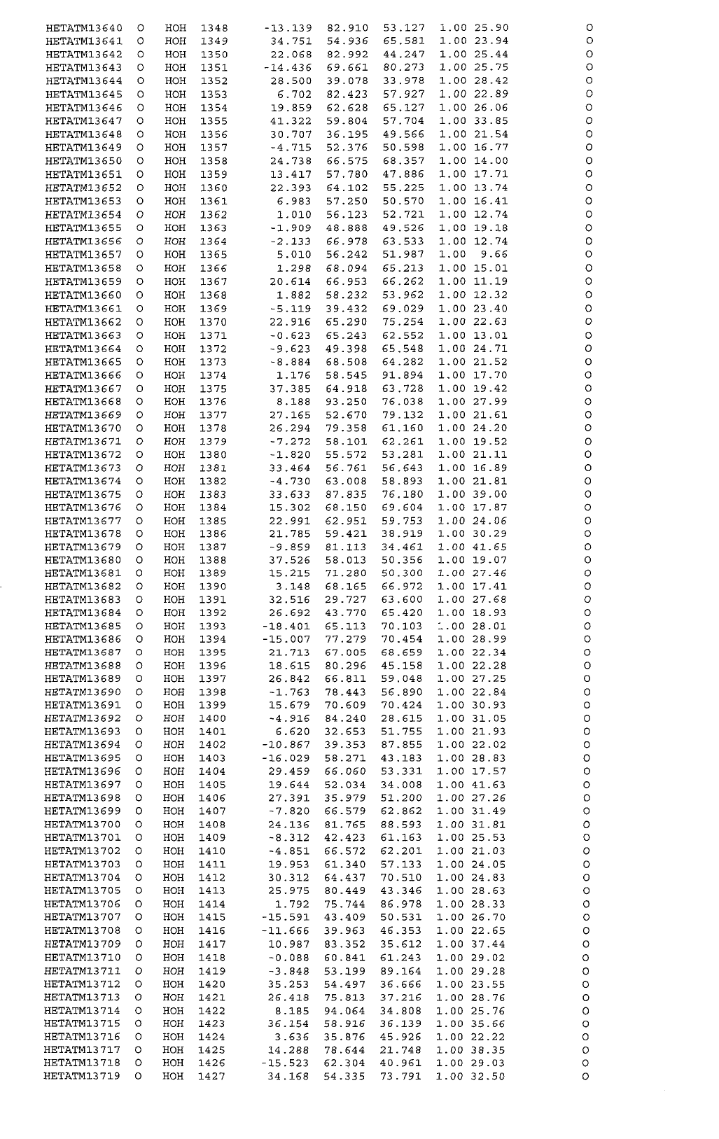

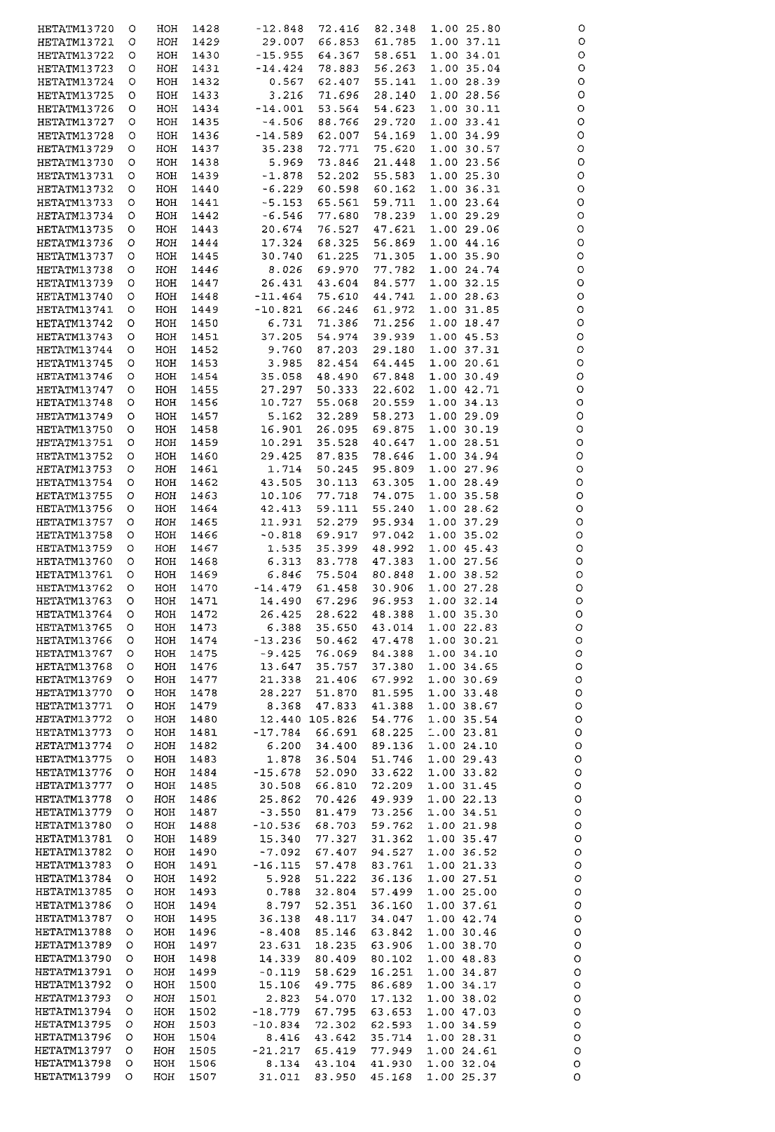

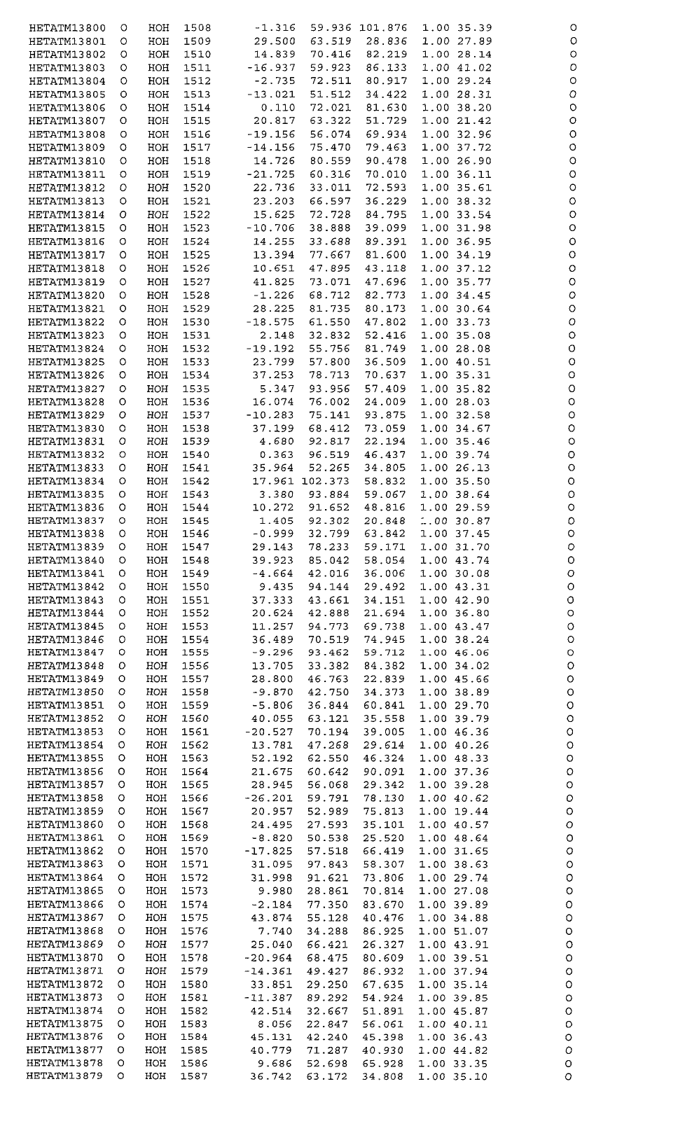

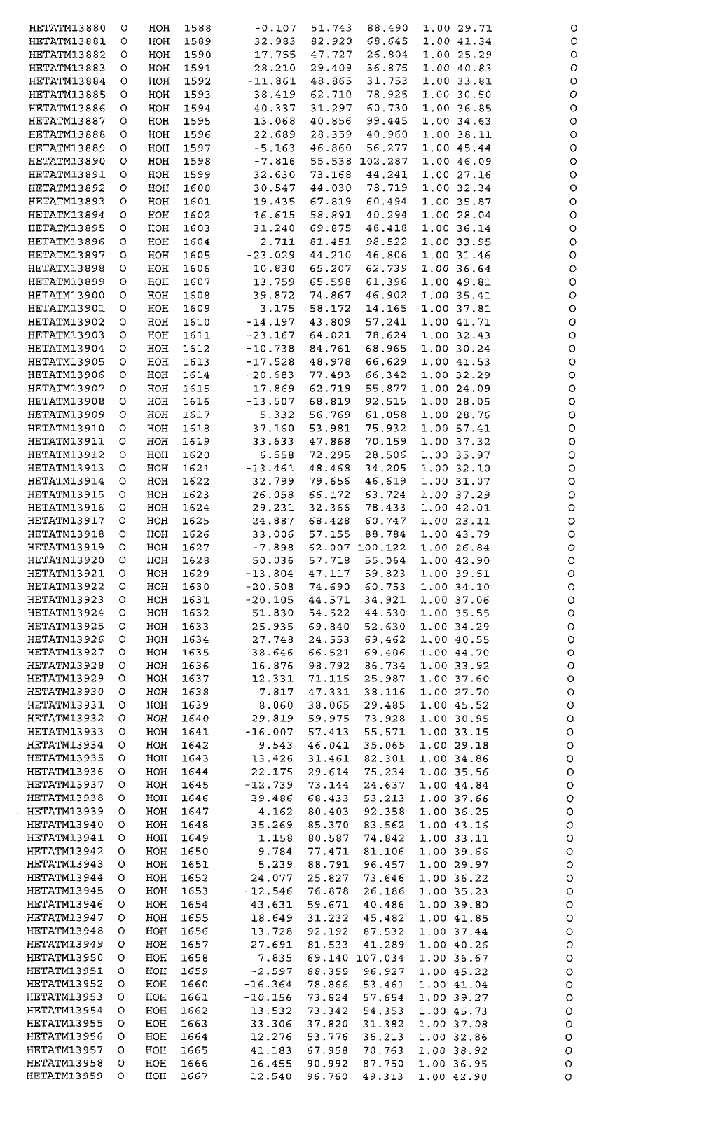

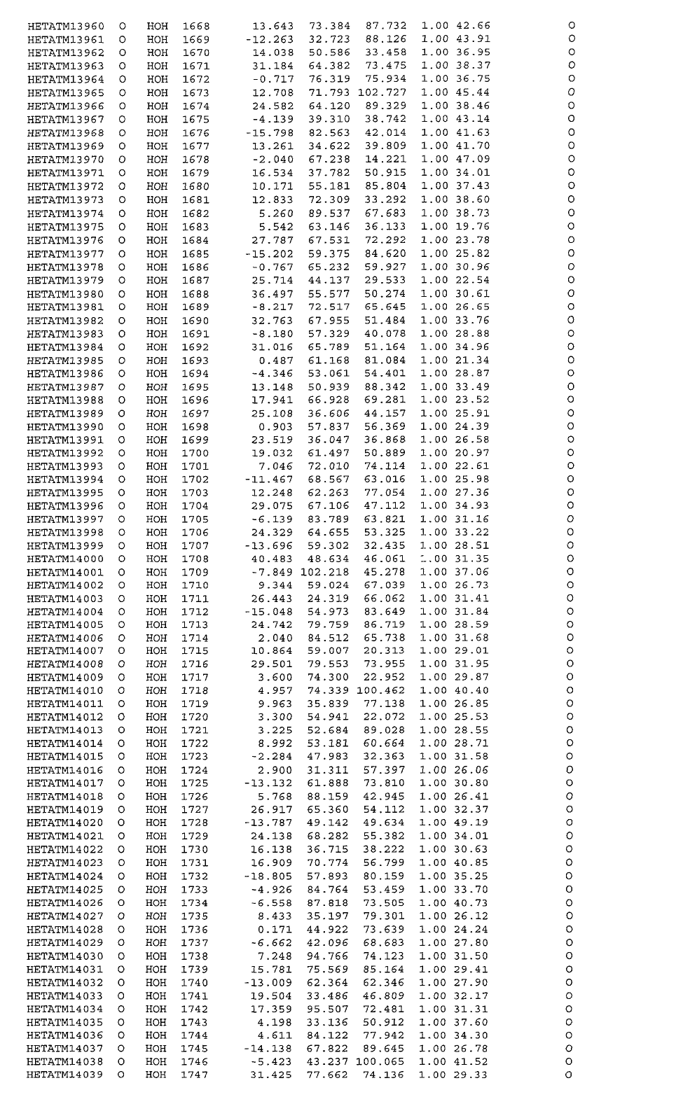

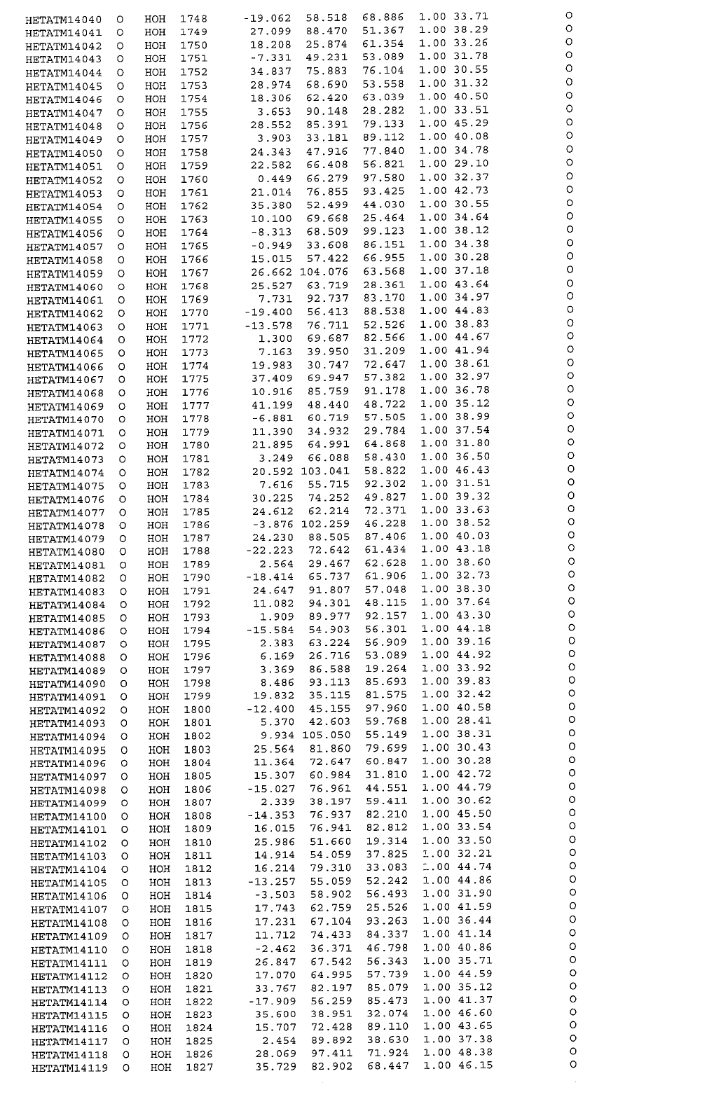

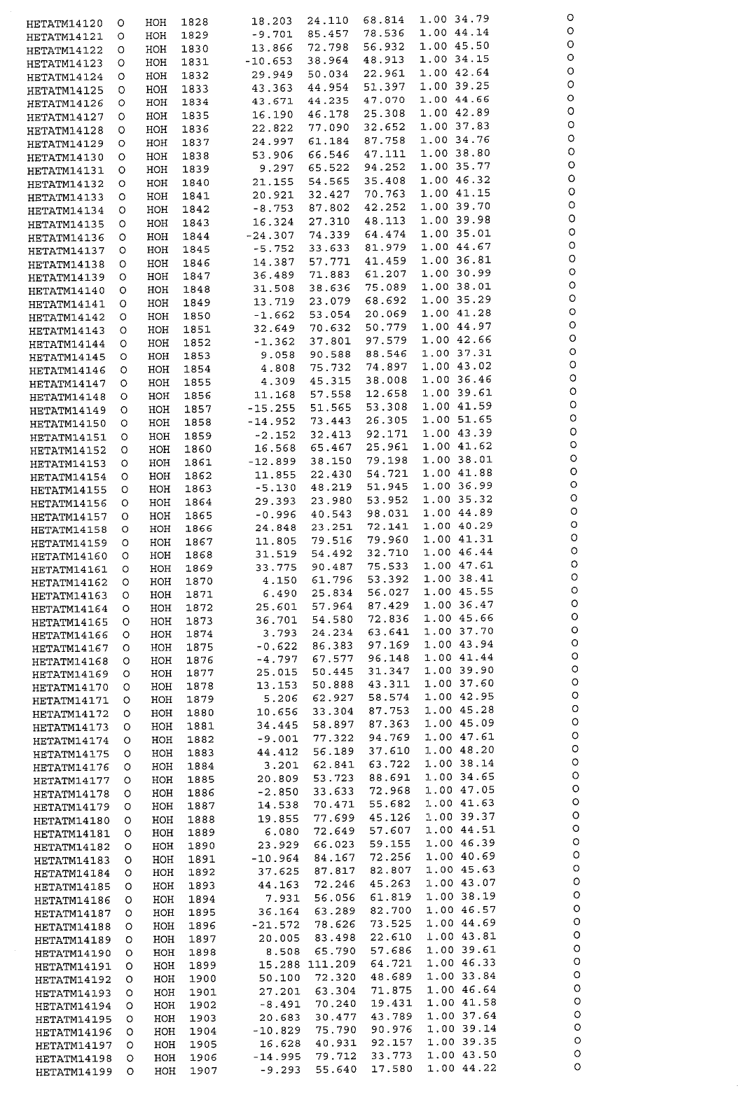

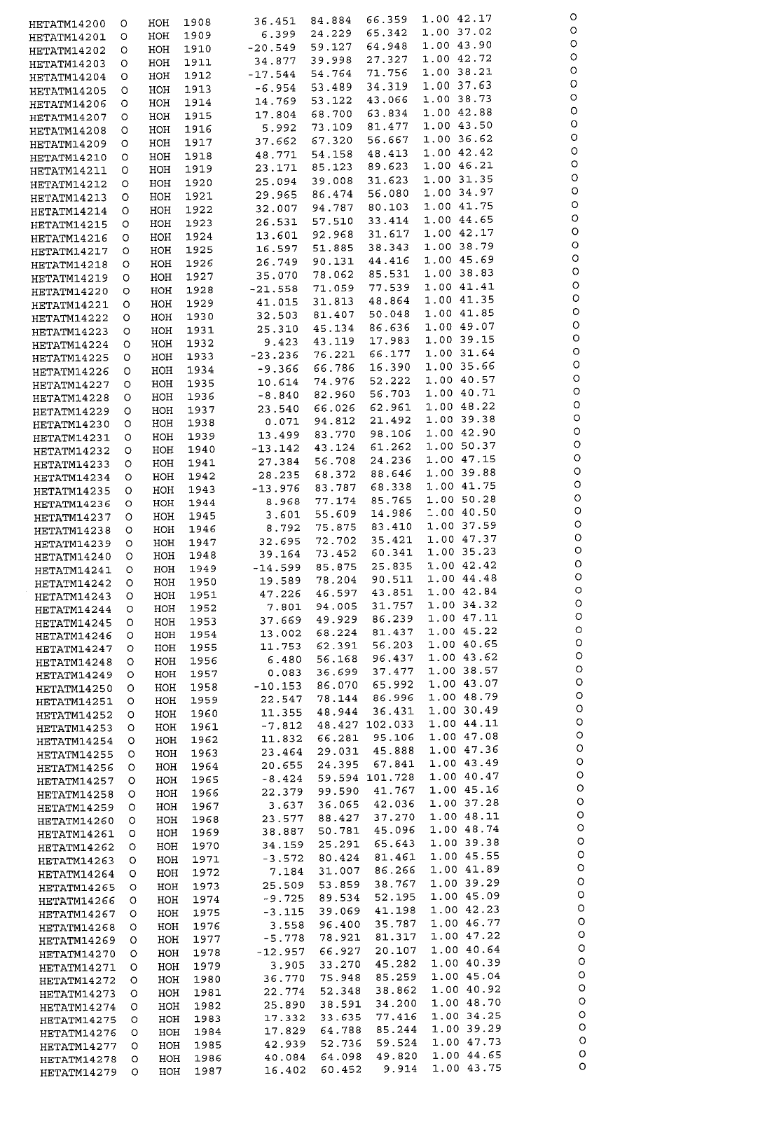

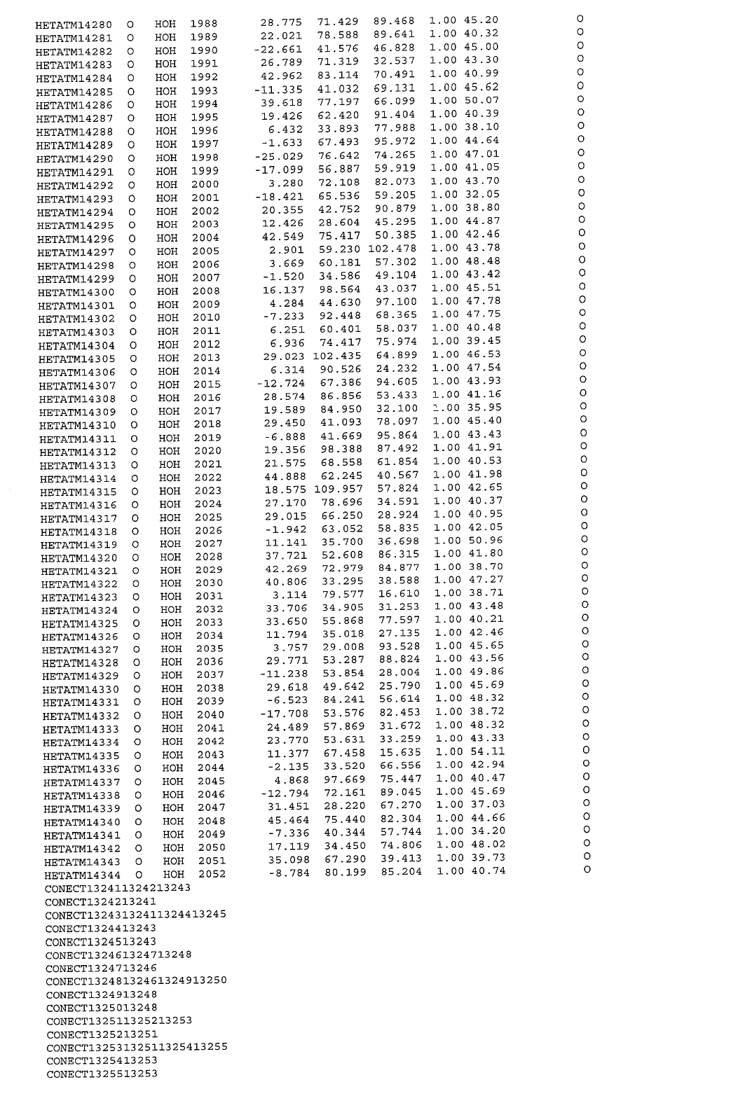

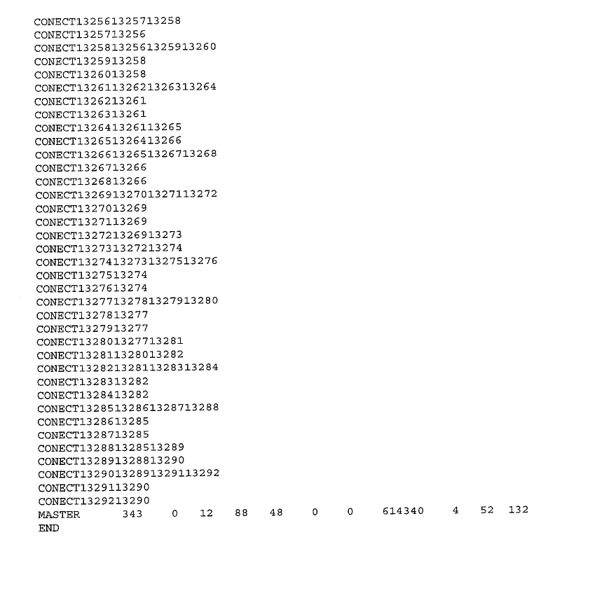

In reference to FIG. 5, FIG. 6 and FIG. 7, those of ordinary skill in the art will understand that the term “structure coordinates” refers to mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

It is further understood in the art that a set of structure coordinates determined by X-ray crystallography is not without standard error. In certain embodiments of this invention, any set of structure coordinates for an enzyme of a microbial glyoxylate shunt, such as ICL or malate synthase, or a homologue or mutant thereof, have a root mean square deviation of protein backbone atoms (N, Cα, C and O) of less than 1.5 angstroms, more preferably of less than 1.0 angstroms, and even more preferably of less than 0.75 angstroms, when superimposed, using backbone atoms, on the structure coordinates provided, such as those in FIG. 5, FIG. 6 and FIG. 7, shall be considered identical.

The term “root mean square deviation” means the square root of the arithmetic mean of the squares of the deviations from the mean. It is a way to express the deviation or variation from a trend or object. For purposes of this invention, the “root mean square deviation” defines the variation in the backbone of a protein from the backbone of an enzyme of a microbial glyoxylate shunt, such as ICL or malate synthase, or an active site or binding pocket portion thereof, as exemplified by the definition of the structure coordinates for ICL described herein and as represented by FIG. 5, FIG. 6 and FIG. 7.

Structure coordinates for enzymes of a microbial glyoxylate shunt, such as ICL or malate synthase, according to the invention, and as represented by FIG. 5, FIG. 6 and FIG. 7, may be modified from the original sets by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates, and any combination of the foregoing and the like.

As ICL and other enzymes of a microbial glyoxylate shunt may crystallize in more than one crystal form, the structure coordinates of such enzymes, as exemplified by ICL, or portions thereof, as provided by this invention are particularly useful to solve the structure of any other crystal forms. The first provided structure coordinates may also be used to solve the structure of ICL mutants, ICL co-complexes, or of the crystalline form of any other protein with significant amino acid sequence homology to any functional domain of ICL.

One method that may be employed for such purposes is molecular replacement. In this method, the unknown crystal structure, whether it is another crystal form of ICL, an ICL mutant, or an ICL co-complex, or the crystal of some other protein with significant amino acid sequence homology to any functional domain of ICL, may be determined using the ICL structure coordinates of this invention as provided in FIG. 5, FIG. 6 and FIG. 7. This method will provide an accurate structural form for the unknown crystal quickly and efficiently.

The term “molecular replacement” therefore refers to a method that involves generating a preliminary model of a crystal of an enzyme of a microbial glyoxylate shunt, such as ICL or malate synthase, whose structure coordinates are unknown, by orienting and positioning a molecule whose structure coordinates are known, e.g., ICL coordinates from FIG. 5, FIG. 6 and FIG. 7, within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal, as is known by those of ordinary skill in the art (Lattman, 1985; Rossmann, 1972, each specifically incorporated herein by reference). Using the structure coordinates of ICL provided by this invention, molecular replacement can thus be used to determine the structure coordinates of a crystalline mutant or homologue of ICL, or of a different crystal form of ICL.

As the present invention provides the ICL coordinates from FIG. 5, FIG. 6 and FIG. 7, the invention permits the use of molecular design techniques to design, select and synthesize chemical entities and compounds, including inhibitory compounds, capable of binding to the active site or other important binding sites of ICL, in whole or in part.

One approach enabled by this invention, is to use the structure coordinates of ICL to design compounds that bind to the enzyme and alter the physical properties of the compounds in different ways. For example, this invention enables the design of compounds that act as competitive inhibitors of the ICL enzyme by binding to, all or a portion of, the active site of ICL. This invention also enables the design of compounds that act as uncompetitive inhibitors of the ICL enzyme. These inhibitors may bind to, all or a portion of, other important binding sites of an ICL already bound to its substrate and may be more potent and less non-specific than known competitive inhibitors that compete only for the ICL active site. Similarly, non-competitive inhibitors that bind to and inhibit ICL whether or not it is bound to another chemical entity may be designed using the structure coordinates of ICL of this invention.

Another design approach is to probe an ICL crystal with molecules composed of a variety of different chemical entities to determine optimal sites for interaction between candidate ICL inhibitors and the enzyme. This may be achieved using the data for the inhibitor complexes of FIG. 6 and FIG. 7.

This invention also provides for the development of compounds that can isomerize to short-lived reaction intermediates in the chemical reaction of a substrate or other compound that binds to ICL, with ICL. Thus, the time-dependent analysis of structural changes in ICL during its interaction with other molecules is enabled. The reaction intermediates of ICL can also be deduced from the reaction product in co-complex with ICL. Such information is useful to design improved analogues of known ICL inhibitors or to design novel classes of inhibitors based on the reaction intermediates of the ICL enzyme and ICL-inhibitor co-complex. This provides a novel route for designing ICL inhibitors with both high specificity and stability.

Another approach made possible and enabled by this invention, is to screen computationally small molecule data bases for chemical entities or compounds that can bind in whole, or in part, to the ICL enzyme. In this screening, the quality of fit of such entities or compounds to the binding site may be judged either by shape complementarity or by estimated interaction energy, as is known those of ordinary skill in the art.

As shown in the working examples herein, ICL and ICL mutants may be crystallized in co-complex with ICL inhibitors. The crystal structures of two such complexes are provides herein (FIG. 6 and FIG. 7). A series of crystal structures may now thus be solved by molecular replacement and compared with the crystal structures of apo-ICL and the two inhibitor complexes already provided. Potential sites for modification within the various binding sites of the enzyme may thus be identified. This information provides an additional tool for determining the most efficient binding interactions, for example, increased hydrophobic interactions, between ICL and a chemical entity or compound.

All of the complexes referred to above may be studied using well-known X-ray diffraction techniques and may be refined versus 2-3 angstrom resolution X-ray data to an R value of about 0.20 or less using readily available computer software, such as X-PLOR (Yale University©, 1992, distributed by Molecular Simulations, Inc.; Blundel & Johnson, 1985, specifically incorporated herein by reference). This information may thus be used to optimize known classes of ICL inhibitors and to design and synthesize novel classes of ICL inhibitors.

The design of compounds that bind to or inhibit ICL according to this invention may involve a consideration of two factors. First, the compound should be capable of physically and structurally associating with ICL, such as by using non-covalent molecular interactions, including hydrogen bonding, van der Waals and hydrophobic interactions and the like. Second, the compound should be able to assume a conformation that allows it to associate with ICL. Although certain portions of the compound will not directly participate in this association with ICL, those portions may still influence the overall conformation of the molecule. This, in turn, may have a significant impact on potency. Such conformational requirements include the overall three-dimensional structure and orientation of the chemical entity or compound in relation to all or a portion of the binding site, e.g., active site or accessory binding site of ICL, or the spacing between functional groups of a compound comprising several chemical entities that directly interact with ICL.

The potential inhibitory or binding effect of a chemical compound on ICL may be analyzed prior to its actual synthesis and testing by the use of computer modeling techniques, as is known to those of ordinary skill in the art. If the theoretical structure of the given compound suggests insufficient interaction and association between it and ICL, synthesis and testing of the compound need not be pursued. Wherein computer modeling indicates a strong interaction, the molecule may then be synthesized and tested to confirm an ability to bind to and inhibit ICL using standard and inventive assays, as described in the present disclosure. In this manner, synthesis of inoperative compounds is avoided.

An inhibitory or other binding compound of ICL may be computationally evaluated and designed by means of a series of steps in which chemical entities or fragments are screened and selected for their ability to associate with the active site or other important areas of ICL.

One of ordinary skill in the art may use any one of several methods to screen chemical entities or fragments for their ability to associate with ICL, and more particularly with the active site of ICL. This process may begin by visual inspection of, for example, the active site on the computer screen based on the ICL coordinates in FIG. 5, FIG. 6 and FIG. 7. Selected fragments or chemical entities may then be positioned in a variety of orientations, or docked, within the active site of ICL. Docking may be accomplished using software such as Quanta and Sybyl, followed by energy minimization and molecular dynamics with standard molecular mechanics forcefields, such as CHARMM and AMBER.

Specialized computer programs may also assist in the process of selecting fragments or chemical entities. These include GRID (Goodford, 1985; available from Oxford University, Oxford, UK); MCSS (Miranker & Karplus, 1991; available from Molecular Simulations, Burlington, Mass.); AUTODOCK (Goodsell & Olsen, 1990; available from Scripps Research Institute, La Jolla, Calif.); and DOCK (Kuntz et al., 1982; available from University of California, San Francisco, Calif.); each of the foregoing references being specifically incorporated herein by reference.

Once suitable chemical entities or fragments have been selected, they can be assembled into a single compound or inhibitor. Assembly may be proceed by visual inspection of the relationship of the fragments to each other on the three-dimensional image displayed on a computer screen in relation to the structure coordinates of ICL. This would be followed by manual model building using software such as Quanta or Sybyl.

Useful programs to aid one of skill in the art in connecting the individual chemical entities or fragments include CAVEAT (Bartlett, 1989; available from the University of California, Berkeley, Calif.); 3D Database systems such as MACCS-3D (MDL Information Systems, San Leandro, Calif., Martin, 1992; HOOK (available from Molecular Simulations, Burlington, Mass.); each of the foregoing references being specifically incorporated herein by reference.

Instead of proceeding to build an ICL inhibitor in a step-wise fashion one fragment or chemical entity at a time as described above, inhibitory or other ICL binding compounds may be designed as a whole or “de novo” using either an empty active site or optionally including some portion(s) of known inhibitor(s), such as those provides herein. These methods include LUDI (Bohm, 1992; available from Biosym Technologies, San Diego, Calif.); LEGEND (Nishibata, 1991; available from Molecular Simulations, Burlington, Mass.); and LeapFrog (available from Tripos Associates, St. Louis, Mo.); each of the foregoing references being specifically incorporated herein by reference.

Other molecular modeling techniques may also be employed in accordance with this invention, such as described by Cohen et al. (1990) and Navia & Murcko (1992); each specifically incorporated herein by reference.

Once a compound has been designed or selected by the above methods, the efficiency with which that compound may bind to ICL may be tested and optimized by computational evaluation. In such methods, the deformation energy of binding may be considered and ICL inhibitors designed with a particular deformation energy of binding desired by one of ordinary skill in the art.

A compound designed or selected as binding to ICL may be further computationally optimized so that in its bound state it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the inhibitor and the enzyme when the inhibitor is bound to ICL, preferably make a neutral or favorable contribution to the enthalpy of binding.

Specific computer software is available in the art to evaluate compound deformation energy and electrostatic interaction. Examples of programs designed for such uses include Gaussian 92, revision C (Frisch, Gaussian, Inc., Pittsburgh, Pa.,© 1992); AMBER, version 4.0 (Kollman, University of California at San Francisco, © 1994); QUANTA/CHARMM (Molecular Simulations, Inc., Burlington, Mass., 1994); and Insight II/Discover (Biosysm Technologies Inc., San Diego, Calif., © 1994). These programs may be implemented, for instance, using a Silicon Graphics workstation, IRIS 4D/35 or IBM RISC/6000 workstation model 550. Other hardware systems and software packages will be known to those of ordinary skill in the art.

Once an ICL-binding compound has been optimally selected or designed, as described above, substitutions may then be made in some of its atoms or side groups in order to improve or modify its binding properties. Generally, initial substitutions are conservative, i. e., the replacement group will have approximately the same size, shape, hydrophobicity and charge as the original group. It will, of course, be understood that components known in the art to alter conformation should be avoided. Such substituted chemical compounds may then be analyzed for efficiency of fit to ICL by the same computer methods described in detail, above.

### III. Pharmaceutical Compositions

Pharmaceutical compositions of the present invention comprise an effective amount of at least a first glyoxylate shunt inhibitor, particularly an isocitrate lyase or malate synthase inhibitor, dissolved or dispersed in a pharmaceutically acceptable carrier, such as an aqueous medium. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

### A. Parenteral

The present invention may be used to prepare anti-microbial, anti-bacterial, anti-mycobacterial and anti-fungal compositions, therapeutics, vaccines and/or cocktails thereof for parenteral administration, e.g., formulated for injection via the intravenous, intramuscular, subcutaneous, transdermal, or other such routes.

The preparation of an aqueous composition that contains a glyoxylate shunt inhibitor as an active ingredient will be known to those of skill in the art in light of the present disclosure. Typically, such compositions can be prepared as injectables, either as liquid solutions or suspensions. Solid forms suitable for using to prepare solutions or suspensions upon the addition of a liquid prior to injection can also be prepared. The preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, particularly bacteria.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations may contain a preservative.

One or more glyoxylate shunt inhibitors can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms, particularly bacteria, can be brought about by various antibacterial agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as desired, followed by filtered sterilization. Generally, dispersions are prepared by incorporating the various sterilized active ingredients into a sterile vehicle which contains the basic dispersion medium and the desired other ingredients from those enumerated above. In the case of sterile powders for the preparation of sterile injectable solutions, the preferred methods of preparation are vacuum-drying and freeze-drying techniques, which yield a powder of the active ingredient plus any additional desired ingredient from a previously sterile-filtered solution thereof.

The preparation of more, or highly, concentrated solutions for intramuscular injection is also contemplated. In this regard, solvents that allow rapid penetration are preferred, delivering high concentrations of the active agents to a small area.

The techniques of preparing pharmaceutical compositions are generally well known in the art, as exemplified by Remington's Pharmaceutical Sciences, Mack Publishing Company, incorporated herein by reference. Upon formulation, solutions will be administered in a manner compatible with the dosage formulation and in such amount as is therapeutically effective. The formulations are easily administered in a variety of dosage forms. Some variation in dosage will necessarily occur depending on the condition of the subject being treated. The person responsible for administration will determine the appropriate dose for the individual subject.

In addition to the compounds formulated for parenteral administration, such as intravenous or intramuscular injection, other pharmaceutically acceptable forms include, e.g., tablets, pills, capsules or other solids for oral administration; time release capsules; suppositories and pessaries; and any other form currently used, including cremes, lotions, mouthwashes, nasal solutions or sprays, aerosols, inhalants, liposomal forms and the like.

### B. Topical Formulations

Glyoxylate shunt inhibitors formulated for topical administration are also contemplated. In a general sense, formulations for topical administration include those for delivery via the mouth, although delivery onto or through the skin may be preferred. “Topical application” is particularly useful for anti-fungal indication. “Topical delivery systems” are generally useful and include transdermal patches containing the ingredient to be administered. Delivery through the skin also includes iontophoresis or electrotransport, if desired.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin include ointments, creams, gels and pastes comprising the glyoxylate shunt inhibitors to be administered in a pharmaceutical acceptable carrier. Formulations for topical use, such as creams, ointments and gels, include oleaginous and/or water-soluble ointment bases, as is well known to those in the art. For example, these compositions may include vegetable oils, animal fats, and more preferably, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates

### C. Therapeutic Kits

This invention also provides therapeutic kits comprising glyoxylate shunt inhibitors for use in anti-microbial treatment methods. Such kits will generally contain, in suitable container means, a pharmaceutically acceptable formulation of at least one glyoxylate shunt inhibitor. The kits may also contain other pharmaceutically acceptable formulations for combined therapy, particularly one or more of a range of conventional anti-microbial and/or anti-fungal therapeutics.

The kits may have a single container (container means) that contains the glyoxylate shunt inhibitor(s), with or without any additional components, or they may have distinct containers for each desired agent. Where combined therapeutics are provided, a single solution may be pre-mixed, either in a molar equivalent combination, or with one component in excess of the other. Alternatively, each of the glyoxylate shunt inhibitor components and other anti-microbial agents may be maintained separately within distinct containers prior to administration to an animal or patient.

When the components of the kit are provided in one or more liquid solutions, the liquid solution is preferably an aqueous solution, with a sterile aqueous solution being particularly preferred. Components of kits formulated for topical administration are also preferred. However, the components of the kit may be provided as dried powder(s). When reagents or components are provided as a dry powder, the powder can be reconstituted by the addition of a suitable solvent. It is envisioned that the solvent may also be provided in another container.

The containers of the kit will generally include at least one vial, tube, flask, bottle, syringe or other container means, into which the inhibitor(s) and any other desired agent, may be placed and, preferably, suitably aliquoted. Where separate components are desired, the kit will also generally contain a second vial or other container into which these are placed, enabling the administration of separated designed doses. The kits may also comprise a second/third container means for containing a sterile, pharmaceutically acceptable buffer or other diluent.

The kits may also contain a means by which to administer the components to an animal or patient, e.g., one or more needles or syringes, or other such like apparatus, from which the formulation may be injected into the animal or applied to a diseased area of the body. The kits of the present invention will also typically include a means for containing the vials, or such like, and other component, in close confinement for commercial sale, such as, e.g., injection or blow-molded plastic containers into which the desired vials and other apparatus are placed and retained.

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLE 1

### Initial Characterization of Isocitrate Lyase in Mycobacteria

The key enzymes of the glyoxylate shunt are isocitrate lyase and malate synthase. The former cleaves isocitrate to succinate and glyoxylate, and the latter condenses glyoxylate with acetyl coenzyme A (acetyl-CoA) to yield malate. The glyoxylate shunt circumvents the loss of two carbon dioxides of the tricarboxylic acid cycle (TCA cycle), thereby permitting net incorporation of carbon into cellular structures during growth on acetate. In addition, even during operation of the TCA cycle, many fatty acids are partially metabolized to acetyl-CoA, thus requiring the presence of isocitrate lyase.

Isocitrate lyase competes with the TCA cycle enzyme isocitrate dehydrogenase for their common substrate isocitrate. By changing the total cellular activity of either of the two enzymes and/or by changing their affinities toward isocitrate, control of carbon flux between the two cycles is achieved. In E. coli, growth on acetate leads to a decrease in NADP⁺-dependent isocitrate dehydrogenase activity caused by the reversible phosphorylation of isocitrate dehydrogenase. The corresponding isocitrate dehydrogenase-kinase is encoded in the same operon as the isocitrate lyase and the malate synthase. The reduction in isocitrate dehydrogenase activity redirects isocitrate into the glyoxylate cycle through the activity of isocitrate lyase. The phosphorylation-dephosphorylation of isocitrate dehydrogenase is believed to regulate entry of the substrate into the glyoxylate shunt. In addition, E. coli isocitrate lyase is inhibited by several metabolites, e.g., succinate, 3-phosphoglycerate or phosphoenolpyruvate, leading to a more subtle control of the carbon flux.

In mycobacteria, isocitrate lyase activity has been reported to increase continuously with the age of the culture in M. tuberculosis H₃₇Rᵥ (Suryanarayana Murthy et al., 1973), but not in M. tuberculosis H₃₇Rₐ or M. smegmatis (Seshadri et al., 1976). Other studies report enhanced glyoxylate cycle enzyme activity under low oxygen tension (Wayne & Lin, 1982) or when the mycobacteria are grown in the presence of acetate (Kannan et al., 1985).

M. tuberculosis contains two genes that encode proteins reported to have isocitrate lyase activity. For ease of reference, the present inventors refer to Rv0467 of the literature as icl (for isocitrate lyase) to distinguish this from other isocitrate lyases of the literature, which are herein termed aceA.

To investigate whether both genes gave rise to active enzymes, the ORFs were cloned based on the DNA sequence of CSU93 and overexpressed the proteins in E. coli. The proteins were purified by the 6His-tag attached to the N terminus. Enzyme assays with the purified proteins indicated that both function as isocitrate lyases.

It was demonstrated at both the protein and enzyme levels that the expression of isocitrate lyase in M. avium and M. tuberculosis increases when acetate or palmitate are the limiting carbon sources and that cultivation in the presence of succinate suppresses isocitrate lyase expression. Preliminary studies with a polyclonal antibody raised against recombinant AceA indicate that it is expressed under similar conditions as Icl only in M. tuberculosis CSU93 and not in M. tuberculosis H₃₇Rᵥ. In addition, the differences in Kₘ and Vₘₐₓ between the two enzymes suggest that AceA fulfills a role subordinate to Icl in the processing isocitrate.

2-D SDS-PAGE analysis of established (8- to 12-day) M. avium infections in murine bone marrow-derived macrophages revealed several polypeptides that were unregulated in expression when compared to bacteria grown in Middlebrook medium. The most notable of these was a protein with an apparent molecular mass of 50 kDa and an isoelectric point of approximately 5.0. This protein was transferred onto polyvinylidene difluoride membranes, and the N-terminal sequence was determined. A polyclonal antibody raised against a synthetic peptide corresponding to this amino acid sequence reacted specifically with a protein of 50 kDa in immunoblots of M. avium grown on acetate. The same antibody recognized a single band of comparable mobility in SDS-PAGE-separated extracts from M. tuberculosis grown in macrophages and M. leprae isolated from SCID mice.

The N-terminal sequence obtained from the 50-kDa protein matched a sequence of 428 amino acids in the mycobacterial genome databases encoded by a gene now termed icl for isocitrate lyase. Further general databank searches with this ORF revealed closest similarity to the isocitrate lyases from Corynebacterium glutamicum and Rhodococcus fascians, with identities to the M. tuberculosis sequence of 79 and 80%, respectively.

These searches also revealed a second ORF in M. tuberculosis with similarity to isocitrate lyase, although its level of identity with enzymes from E. coli (35%) and M. tuberculosis Icl (37%) is lower. In M. tuberculosis H₃₇Rᵥ this second isocitrate lyase gene (named aceA) has two overlapping ORFs (aceaa and aceAb) that share a single base pair of the 3′ end of aceAa and the 5′ end of aceAb. However, in M. tuberculosis CSU93 aceA appears to be a single ORF. The recent progress of the sequencing projects from M. avium, M. tuberculosis, and M. leprae show that both the icl and the aceA ORFs exist as two distinct genes present in single copies in all of those strains, with a sequence identity of ca. 80% between species.

To confirm that the Icl and AceA are active isocitrate lyases, the two ORFs of M. tuberculosis (CSU93) were expressed in E. coli by using a pET-based vector. The recombinant proteins were purified to approximately 90% in a one-step procedure by using a metal-affinity resin. Recombinant Icl reacted with the antibody generated against the N-terminal amino acid sequence AceA reacted with an antipeptide antibody generated against an internal sequence of the aceA ORF. These blots confirm the identity of the recombinant enzymes and their corresponding ORFs.

The specific activity of the purified enzyme for isocitrate was 1.3 μmol/min/mg of protein. The Kₘ of the purified recombinant Icl for threo D-(s) isocitrate was determined to be 145 μM by using a Hanes-Woolf plot.

The inhibition of Icl activity by several compounds, known to be effective against various isocitrate lyases, was examined. Itaconate, itaconic anhydride, bromopyruvate, and 3-nitropropionate were found to be the most potent inhibitors, with inhibition constants of 120, 190, 120, and 3 μM, respectively. Interestingly, at saturating substrate conditions succinate did not reveal any inhibitory effect on the recombinant enzyme. Oxalate and malate were also shown to inhibit the activity to approximately 50% at 5 mM inhibitor concentration. 3-Phosphoglycerate, 6-phosphogluconate, fructose-1,6-bisphosphate, and malonic acid had no inhibitory effect. Most of the effectors can be classified as structural analogs of the reaction products succinate (itaconate, itaconic anhydride, and 3-nitropropionate) or glyoxylate (3-bromopyruvate and oxalate).

In the absence of divalent cations, only negligible activity was measured for the purified Icl, whereas addition of Mg²⁺ or Mn²⁺ supported enzyme activity. Mg²⁺ at 5 mM was found to be the most effective cation. Mn²⁺ was able to replace Mg²⁺, yielding 39% of the activity obtained with Mg²⁺.

The Icl activity was assayed for its pH dependence. The optimal pH for the assay of Icl activity was found to be 6.8 with a MOPS buffer.

Enzyme activity was stable for several months when the purified protein was frozen in liquid nitrogen and stored at −80° C. Even upon thawing and storage at 4° C., only minimal activity was lost after 2 months. Addition of glycerol and the presence of a reducing agent in the purification buffer appeared to be necessary to retain activity of the purified recombinant protein.

Recombinant AceA also showed isocitrate lyase activity after purification, the Kₘ of the recombinant protein for threo D-(s) isocitrate was determined to be 1.3 mM by using a Tricine-HCl buffer at pH 7.5. This Kₘ is approximately 10-fold higher than that of Icl. The Vₘₐₓ of the purified enzyme for threo D-(s) isocitrate was 0.41 μmol/min/mg, ca. three times slower than Icl.

Effects of carbon sources on Icl and AceA induction. In most organisms, the expression of isocitrate lyase activity is dependent on the carbon source in the growth medium. To elucidate whether the induction of Icl and AceA expression and activity in M. avium and M. tuberculosis is also dependent on nutrients, crude bacterial extracts were assayed for both isocitrate lyase activity and expression.

The specific total activity of the isocitrate lyase in M. avium varied widely depending on the primary carbon source. The lowest level of induction was observed when cells were grown on succinate or glucose, with specific activities of 12.3±0.35 and 97.6±0.2 nmol/min/mg of protein, respectively. Growth on acetate or on palmitate led to high levels of induction (439.6±0.2 and 1.193.4±0.2 nmol/min/mg of protein, respectively). It was also determined whether either acetate or palmitate could still upregulate isocitrate lyase activity when the cultures were given an alternate carbon source.

M. avium cells were harvested in mid-log phase to avoid depletion of any of the carbon sources. When cultures were grown on palmitate with glucose, the levels of enzyme activity were about half that of acetate-grown cultures (217.5±0.51 nmol/min/mg of protein). Less activity was demonstrated with M. avium cultivated on acetate plus glucose at 84.7±0.35 nmol/min/mg of protein. Growth of M. avium on a combination of succinate and acetate or on succinate and palmitate in minimal medium, however, did not result in any upregulation of the enzyme. Thus, the presence of an alternate carbon source does not completely repress induction of the isocitrate lyase unless succinate is present.

Because the total isocitrate lyase activity is the product of two discrete enzymes, the relative levels of Icl and AceA protein were examined by immunoblotting comparable amount of SDS-PAGE separated extracts from bacilli grown under differing conditions. It was found that the activity levels correlated with the relative amount of Icl protein detected in immunoblots. Expression of AceA in M. avium is apparently only upregulated by acetate and not by palmitate, while succinate, again, repressed expression of this enzyme completely. The data indicate strongly that the primary regulation of carbon flux into the glyoxylate pathway is at the level of isocitrate lyase expression. The pronounced effect of succinate on the Icl and AceA production is likely attributable to feedback inhibition since succinate is one of the products of isocitrate lyase activity.

The levels of expression of Icl and AceA in M. tuberculosis CSU93 and H₃₇Rᵥ was also examined under different culture conditions. However, M. tuberculosis is more fastidious than M. avium and would not grow on minimal medium that was not supplemented with Casamino Acids in addition to acetate or palmitate, although the latter carbon sources were limiting for growth. As a result, it is unknown whether the activity levels obtained were maximal. By according glucose-grown bacteria an arbitrary value, it was found that palmitate induced isocitrate lyase activity (threefold) to a greater extent than acetate (twofold) and that succinate repressed the enzyme activity below levels of detection. Glucose, on the other hand, could not repress expression of activity when given together with acetate or palmitate (these remained two- to threefold). Once again these data were substantiated by immunoblots with anti-Icl and anti-AceA antibodies.

In contrast to M. avium, AceA production in strain CSU93 increased when the cells were grown on palmitate. Interestingly, when grown under the same conditions, only strain CSU93 and not H₃₇Rᵥ expressed levels of AceA detectable by immunoblots. The apparent lack of expression of this enzyme in H₃₇Rᵥ may indicate that the ORF(s) Rv1915 and Rv1916 do not give rise to a product.

### EXAMPLE 2

### Persistent Infection of M. tuberculosis Requires the Glyoxylate Shunt

### A. Methods

1. Mycobacterial Strains and Growth Conditions. M. tuberculosis (Erdman and CSU93) were passaged once through mice and stored in aliquots at −80° C. M. smegmatis mc²155 was colony-purified and stored in aliquots at −80° C. Mycobacteria were grown in 7H9 broth or 7H10 agar, supplemented with 10% OADC, 0.5% glycerol, 100 μg ml⁻¹ cycloheximide, and 0.1% Tween-80. Antibiotics were hygromycin at 50 μg ml⁻¹ or kanamycin at 25 μg m⁻¹. Defined carbon medium was M9 agar (DifCo) supplemented with glucose, sodium acetate, or methyl palmitate at 0.1%.

2. Isolation and Complementation of an icl Mutant of M. smegmatis. M. smegmatis mc²155 was mutagenized with 2.5% ethyl methane sulfonate (Sigma) in 0.1 M phosphate buffer (pH 7) for 90 min, washed, recovered in 7H9 broth for 6 hr at 37° C., and plated for colonies on 7H10 agar. Two icl mutants were identified as colonies that failed to grow on M9 agar +0.1% sodium acetate unless transformed with pJM007, which contained a fusion of the E. coli aceA gene to the mycobacterial heat shock promoter in pMV261. The M. tuberculosis icl gene was isolated from a mycobacterial genomic library by marker rescue.

3. Disruption of icl in M. tuberculosis. A 2.7 kbp BamHI-ClaI genomic fragment spanning the M. tuberculosis icl gene was inserted into pYUB631 and a 685 bp Xhol fragment internal to icl was replaced with the hyg hygromycin resistance cassette. The deletion/disruption eliminated the codons for aa 65-290 of the 428 aa ICL protein and abolished biological activity. The Δicl::hyg allele was substituted for icl in the M. tuberculosis chromosome by allelic exchange, and confirmed by Southern blot probed with a 981 bp SacII fragment from the M. tuberculosis icl ORF.

4. M. tuberculosis Growth and Persistence in Mice. C57BL/6J, 129SvEv, Balb/c, and IFNγ^{-|-} mice were purchased from Jackson Laboratories. Frozen stocks of M. tuberculosis strains were thawed, diluted to ˜10⁷ or ˜10⁶ cfu ml⁻¹ in PBS/Tween, and sonicated. Mice were infected by tail-vein injection of 0.1 ml (˜10⁶ or ˜10⁵ cfu) of the bacterial suspension. Infected mice (n=4 or 5 per group) were anaesthetized by intraperitoneal injection of 0.1 ml of 10 mg ml⁻¹ Nembutal and euthanized by dislocation of the cervical vertebrae. Organs were transferred to plastic Tekmar bags with 10 ml PBS/0.1% Tween-80 and homogenized in a Tekmar Stomacher. Organ homogenates were diluted and plated on 7H10 agar. Colonies were scored after 3-4 weeks at 37° C. Portions of tissues were fixed in phosphate-buffered formalin (10%) and photographed.

5. Reporter Construction and Analysis of ICL::GFP Expression. pICL::GFP was derived from pMV262 by substitution of the icl promoter (310 bp 5′ of the start codon of icl) and the icl ORF fused at the 3′ end to the mut2 green fluorescent protein gene. The icl::gfp fusion gene was also used to replace the chromosomal icl gene through homologous recombination (cICL::GFP) in M. tuberculosis CSU93. Quantification of bacterial ICL::GFP expression in infected macrophages was obtained by flow cytometry.

Murine bone-marrow macrophages were non-activated or activated with IFN-γ (100 U ml⁻¹, 16 hrs) and LPS (2 μg ml⁻¹, 2 hrs). Infected monolayers were washed 3× with PBS and lysed with 0.1% Saponin. Intact cells and nuclei were pelleted (250×g, 5 min) and the supernatants containing bacteria were collected. Bacteria were pelleted (2,000×g, 20 min), fixed with 4% paraformaldehyde in PBS for 30 min, washed once with PBS/0.05% Tween-80/0.1% BSA, and resuspended in PBS/Tween. Flow cytometry was done with a FACScalibur cytometer (Becton Dickinson Immunocytometry Systems) and data were collected on 5×10⁴ bacterium-sized particles per sample. Background fluorescence was determined by analysis of macrophages infected with wild-type bacteria. Relative fluorescence was expressed as fold induction over background.

6. Survival of Δicl Bacteria in Resting and Activated Macrophages. Murine bone-marrow macrophages were either non-activated or activated with rIFN-γ (50 U ml⁻¹, 16 hr). This activation protocol promotes acidification of bacteria-containing vacuoles without strong induction of inducible nitric oxide synthase (NOS2), a potent antimicrobial response. The M. tuberculosis Erdman strains (wild-type, Δicl mutant, and Δicl mutant complemented with pICL::GFP) were added at a multiplicity of 10:1 (bacteria per macrophage) to duplicate wells, incubated for 2 hr, washed, and fresh medium was added. Samples were collected at 6, 24, 72 and 120 hr time points by lysing infected cells with 0.5% Tween-20. Lysates were diluted in PBS/Tween and plated on 7H10 agar. Colonies were scored after 3-4 weeks at 37° C.

### B. Results and Discussion

Although of interest for study, prior to the present invention, the mechanisms by which intracellular pathogens acquired essential nutrients remained an area of considerable ignorance. Biochemical studies suggest that fatty acids might be a major source of carbon and energy for metabolism of M. tuberculosis in chronically infected lung tissues. Two pathways are required for fatty acid utilization by bacteria: the β-oxidation cycle, and the glyoxylate shunt. The glyoxylate shunt is essential for carbon anaplerosis in the Krebs cycle during growth on C₂ substrates such as fatty acids, which are the only abundant C₂ carbon sources in mammalian tissues. The glyoxylate shunt is widespread among prokaryotes, lower eukaryotes, and plants, but it is absent in vertebrates.

Earlier studies of the inventors revealed that expression of isocitrate lyase, an enzyme of the glyoxylate shunt, is upregulated during infection of macrophages by Mycobacterium spp. (Example 1; Hoener zu Bentrup et al., 1999, specifically incorporated herein by reference). In the present example, genes encoding isocitrate lyase activity in mycobacteria were identified by a genetic approach. A mutant of M. smegmatis was isolated that failed to grow on C₂ substrates unless rescued by the E. coli aceA gene encoding isocitrate lyase. M. tuberculosis expresses two enzymes with isocitrate lyase activity, ICL (Rv0467) and AceA (Rvl915/6). However, only the gene encoding ICL, and not AceA, was able to rescue the M. smegmatis icl mutant for growth on C₂ substrates. The icl gene was disrupted in the virulent Erdman strain of M. tuberculosis via allelic exchange and the Δicl mutation was confirmed by Southern blotting.

In E. coli and Salmonella typhimurium, induction of the fatty acid catabolic regulon is essential for survival in stationary phase (Farewell et al., 1996; Spector et al., 1999), suggesting a similar role for M. tuberculosis. In one study, ICL activity was induced when well-aerated cultures of M. tuberculosis grew to saturation (Suryanarayana Murthy et al., 1973), suggesting a role for ICL in stationary phase survival. However, the present data show no phenotype for the Δicl mutant of M. tuberculosis in log phase or stationary phase in vitro. In a second study, M. tuberculosis ICL was induced by oxygen limitation (Wayne et al., 1982), and it was suggested that ICL might contribute to adaptation to hypoxia. However, the present data also show that wild-type and Δicl strains of M. tuberculosis were indistinguishable phenotypically when cultured in hypoxic or anoxic atmospheres.

The contribution of ICL to in vivo metabolism of M. tuberculosis was assessed by infecting immune-competent mice with wild-type or Δicl bacteria. The Δicl mutation had little effect on bacterial growth during the acute phase of infection (0-2 wk): Mean doubling times were 50.8 hr for wild type vs. 52.6 hr for Δicl bacteria. However, from 2 wk onwards, the Δicl mutant was eliminated progressively from the lungs and extrapulmonary organs, with a >1.5 log decline in bacterial burden by 16 wk. In contrast, the peak bacterial load of wild type M. tuberculosis was maintained for the duration of the study. There is some evidence that the plateau in bacterial numbers during persistent infection reflects bacterial stasis rather than balanced growth and killing, but this point remains controversial. The persistence defect caused by the Δicl mutation was not due to polar effects on neighboring genes because the wild type phenotype was restored by a plasmid expressing ICL.

The reduced ability of the Δicl mutant to sustain an infection was accompanied by attenuated virulence. At 2 wk post-infection, lungs of mice infected with wild-type and Δicl bacteria showed macroscopic lesions that were comparable in size and number, reflecting the similarity in bacterial loads at 2 wk. By 16 wk, however, disease progression had diverged dramatically; the lungs of mice infected with Aicl bacteria showed little change between 2 and 16 wk, whereas the lungs of mice infected with wild-type bacteria became grossly inflamed and enlarged, with numerous expanding and coalescing tubercles. Attenuated virulence of the Δicl mutant was also demonstrated by infection of Balb/c mice, which exhibit a more rapid disease progression. Balb/c mice infected with wild-type bacteria succumbed between days 68-113 (average, 88 days), whereas all mice infected with Δicl bacteria were surviving at day 168.

These data show that ICL is important for survival of M. tuberculosis in the lungs of mice during the persistent phase of infection. In contrast, ICL is dispensable for bacterial growth in the acute phase of infection. These observations indicate a “C₂ shift” in bacterial carbon metabolism concomitant with the host's response to infection. The emergence of adaptive immunity in vivo is paralleled by the accumulation of inflammatory macrophages in the lungs. In resting macrophages, pathogenic mycobacteria replicate exponentially within vacuoles that are blocked in maturation, acidification, and fusion with lysosomes. In contrast, bacteria that enter activated macrophages confront a more hostile environment, in which vacuole maturation and acidification, restricted access to nutrients, and reduced oxygen tension may all restrict bacterial growth. These environmental changes could affect the metabolism of intracellular mycobacteria. Therefore, the following studies were conduced to determine whether the activation status of the infected macrophage influenced the expression of and requirement for ICL.

To analyze the effect of macrophage activation on ICL expression, a bifunctional ICL::GFP fusion was constructed that was fluorescent yet retained ICL activity. The icl::gfp gene was expressed from the icl promoter on a plasmid or by replacement of the chromosomal icl gene via homologous recombination. Expression of ICL::GFP was induced by palmitate and repressed by succinate, consistent with the inventors' results for the wild-type ICL enzyme. The relative levels of ICL::GFP were evaluated by fluorescence microscopy and flow cytometry following infection of macrophages that were non-activated or pre-activated with interferon-y (IFN-γ) and lipopolysaccharide (LPS). In both resting and activated macrophages, elevated levels of ICL::GFP were observed shortly after infection. However, the levels of ICL::GFP in the bacilli returned to background by 24 hr in resting macrophages, and yet remained elevated in activated macrophages for the duration of the study.

The observation that ICL expression is linked to the activation status of the infected host cell suggested that ICL might be more important for bacterial survival in activated than in resting macrophages. This idea paralleled the inventors' finding that ICL is required for bacterial survival specifically during the persistent phase of infection in mice. Therefore, the phenotype of the M. tuberculosis Δicl mutant in resting versus activated macrophages was compared at 6, 24, 72, and 120 hr post-infection. All preparations showed a marked decrease in bacterial numbers between 6 and 24 hr irrespective of bacterial strain or host cell status.

However, after 24hr, the different bacterial phenotypes became apparent. In resting macrophages, the Δicl mutant showed a modest reduction on viability in comparison to the wild type and pICL::GFP-complemented Aicl strains. In activated macrophages, however, the percentage survival of the Δicl mutant was markedly impaired (1.2%) relative to both the wild-type (41%) and complemented Δicl mutant (52%). This trend was confirmed in three independent studies.

The inventors' observation that the phenotype of Δicl bacteria was more pronounced in activated than resting macrophages indicates a direct link between the immune status of the host and the requirement for ICL. Further evidence for this association comes from the restored virulence of the Δicl mutant on infection of immune-deficient, interferon-γ (IFN-γ) knockout mice. The Δicl mutant grew progressively in the lungs of IFN-γ^{1|1} mice and infection was rapidly lethal. A slight residual phenotype was noted for the Δicl mutant in the IFN-γ^{-|1} mice; bacterial growth slowed after 10 days of infection and lethality was somewhat delayed, which might be due to other macrophage-activating cytokines such as tumor necrosis factor α (TNF-α). These findings thus establish an intriguing link between the immune status of the host and the metabolism of M. tuberculosis in vivo that is meditated through activation of parasitized macrophages.

Persistence of bacteria and chronicity of infection are hallmarks of tuberculosis. Human patients with chronic tuberculosis are thought to harbor bacteria in various metabolic states, ranging from active cell growth and division to stationary phase. Conventional anti-TB and other antimicrobial drugs target processes required for bacterial cell growth and division, such as cell wall biogenesis and chromosome replication. Poor activity against slow- or non-growing bacteria is thought to be a major reason why conventional drugs take so long to eradicate infection. Therefore, the demonstration in the present example that ICL promotes persistence of infection by enhancing bacterial survival within inflammatory macrophages makes ICL and other enzymes of the glyoxylate shunt attractive and novel targets for chemotherapy.

The identification and development of ICL inhibitors as novel drug candidates with preferential activity against persistent bacteria is now possible, given the motivation provided by the present data, using standard screening and other biochemical techniques. Although the development of ICL inhibitors does not rest solely on three-dimensional structural information, the discovery and/or design of ICL inhibitors is further facilitated by the solution of the three-dimensional structure of M. tuberculosis ICL in association with the prototypic inhibitors 3-bromopyruvate and 3-nitropropionate, as provided in Example 3.

### EXAMPLE 3

### Structure of Isocitrate Lyase from M. Tuberculosis

### A. Methods

1. Inhibition Studies. Growth of the wild type M. smegmatis mc²155 or ICL mutant (Δicl) strain complemented with a plasmid containing ICL (pICL1) was monitored in M9 medium with glucose or acetate as the carbon source and in the presence of drug discs soaked in varying concentrations (30 mM or 60 mM) of the ICL inhibitors.

2. Cloning and Purification. The open reading frame for ICL was amplified from the genomic DNA using polymerase chain reaction. The construct was made by cloning the NdeI-HindIII fragment in to pET30(b) and expressed in E. coli using a T7 polymerase based system. The enzyme was purified by anion exchange chromatography followed by gel filtration using a buffer containing 50 mM Tris-HCl (pH 8.0), 100 mM NaCl, 1 mM DTT and 0.1 mM EDTA. The C191S mutant was generated by PCR™ mutagenesis and purified similarly. Selenomethionylated ICL was produced by standard methods, as described in Hendrickson et al. (1990).

3. Crystallization and Data Collection. The hexagonal crystals of the enzyme were obtained by vapor diffusion method using 100 mM HEPES pH 7.5, 1.4 M sodium citrate as the precipitation buffer. Selenomethione substituted protein was crystallized under the same conditions. An orthorhombic crystal form was obtained by modifying the ICL enzyme with 3-bromopyruvate and after incubation with glyoxylate and 3-nitropropionate. These crystals appeared in the presence of 0.1 M Tris-HCl (pH 8.0), 0.2 M sodium acetate, 20-30% (w/v) PEG4000. MAD data were measured for a selenomethione containing crystal on a ADSC Q1 CCD at the 14-BM-D beam line at the Advanced Photon Source (APS), Argonne National Laboratory.

The data on native hexagonal and 3-bromopyruvated orthorhombic crystals were collected on a 3k×3k CCD at the 19-ID beam line at the APS. All crystals were flash-frozen in liquid nitrogen on 0.1 mm cryoloops (Hampton) and were maintained throughout data collection in a cryostream at 100 K.

Data for the ICL-nitropropionate complex was collected on an ADSC Q4 CCD at the 14-BM-C beam line at the APS. Diffraction amplitudes from crystals of the complex were indexed and integrated with DENZO or HKL2000 and scaled with SCALEPACK (Otwinowski and Minor, 1997).

4. Structure Determination and Refinement. The program SOLVE (Terwilliger and Berendzen, 1999) identified 12 Se sites with a mean figure of merit (FOM) of 0.57 and a score of 64. Maps improved to a FOM of 0.84 by solvent flattening. The final structure of the hexagonal crystal form was built using maps obtained by merging the MAD phases with the 2.0 Å native data and subsequent phase extension and density modification (mean FOM 0.91) using CNS (Brunger et al., 1998). The structure of the 3-bromopyruvate complex was solved by molecular replacement, using the program AMoRe program (Navaza, 1994), the CCP4 program package (Collaborative Computational Project, Number 4, 1994) with the structure of the hexagonal form as a search model.

The model for both native crystal and the 3-bromopyruvate complex were built in O (Jones et al., 1991), and all subsequent refinement steps were performed in CNS. Anisotropic overall B-factors and a bulk solvent mask were used throughout the final rounds of refinement and model building. A total of 93.3% of the amino acids in the native crystal and 93.7% of the amino acids in the 3-bromopyruvate complex are found in the most favored regions, and none are found in disallowed regions of the Ramachandaran plot drawn by PROCHECK (Laskowski et al., 1993). The refined structure of the C191S mutant with 3-nitropropionate had similar statistics.

### B. Results and Discussion

1. Activity of ICL inhibitors. The requirement of ICL to a persistent infection makes it an attractive target for drug discovery (Example 2; McKinney et al., 2000). Prior to the present invention, screening for inhibitors against ICL was extremely complex and used infected macrophages or mice. The approach of the present inventors is to screen compounds, first for inhibitory activity against the enzyme and then for mycobacterial survival when the bacteria are grown on a C₂ carbon source.

The inventors have now developed a rapid drug screening strategy using a Δicl M. smegmatis mutant complemented with the M. tuberculosis icl gene. Both wild type and complemented bacteria were plated on minimal plates containing either glucose or acetate as the limiting carbon source. Discs of filter paper soaked with variable concentrations of ICL inhibitors were added to the plates and inhibition was graded based on the radius of the inhibition zone. In these assays, 3-nitropropionate (Schloss and Cleland, 1982) and 3-bromopyruvate (Ko and McFadden, 1990) (FIG. 1B and FIG. 1C), both of which inhibit ICL in vitro (Hoener zu Bentrup et aL, 1999), had no effect on the bacteria grown on glucose (FIG. 1D and FIG. 1E), but were inhibitory to mycobacterium grown on acetate (FIG. 1F and FIG. 1G), albeit at high concentration. 3-Nitropropionate and bromopyruvate are shown to inhibit M. avium ICL (Kᵢ=3 μM and 120 μM, respectively) (Schloss and Cleland, 1982).

The present results indicate that 3-nitropropionate and 3-bromopyruvate are good starting candidates for structure based drug design. As the majority of screens for antimycobacterial agents are conducted on bacteria grown in rich media, conditions where the glyoxylate shunt is dispensable, compounds that target this key pathway for persistent infection of inflammatory macrophages would not have been identified using the prior methodology.

The crystal structures of the apo ICL from Aspergillus nidulans and a complex of it with glyoxylate (Protein Data Bank accession code lDQU) were recently reported (Britton et al., 2000). As the active site region was disordered in these structures, several important questions were left unanswered, meaning that the prior structural information had little utility in drug design. In contrast, the present inventors have solved the structure of native ICL from M. tuberculosis using the multiwavelength anomalous dispersion (MAD) method (Table 1). In addition, the structures of ICL after reaction with 3-bromopyruvate and of the C191 S mutant in complex with 3 -nitropropionate and glyoxylate were solved and refined at resolutions of 1.8 Å and 2.25 Å, respectively.

TABLE 1
CRYSTALLOGRAPHIC DATA

| Space Group | Hexagonal (P6₅22) | | | | Orthorhombic(P2₁2₁2₁) | |
|---|---|---|---|---|---|---|
| Subunits per asymmetric unit | 2 | | | | 4 | |
| Solvent content | 66% | | | | 40% | |
| | a = 130.2 Å | | | | a = 74.3 Å | a = 74.3 Å |
| Unit Cell dimensions | b = 130.2 Å | | | | b = 129.0 Å | b = 129.0 Å |
| | c = 288.4 Å | | | | c = 166.2 Å | c = 166.9 Å |
| Crystal | Edge | Peak | High | Native | 3-Bromopyruvate | 3-Nitropropionate: |
| | | | | | | GA: C191S |
| Xray source | APS-14-BM-D | APS-14-BM-D | APS-14-BM-D | APS-19-ID | APS-19-ID | APS-14-BM-C |
| Wavelength (Å) | 0.9798 | 0.9795 | 0.9571 | 0.9791 | 1.0089 | 1.000 |
| Maximum Resolution | 3.0 | 3.0 | 3.0 | 2.0 | 1.8 | 2.25 |
| Reflections Unique (Total) | 44652(216086) | 42801(154009) | 42075(148773) | 97699(1082279) | 142726(691246) | 72007(296878) |
| Completeness (%) | 84.4(58.7) | 80.4(52.8) | 78.9(49.3) | 99.8(100.0) | 96.3(94.1) | 92.8(79.8) |
| R_{sym}¹(%) | 10.2(19.0) | 9.9(17.0) | 9.3(17.5) | 7.6(37.0) | 4.6(27.9) | 5.0(12.2) |
| <I/σI> | 9.2 | 9.9 | 9.6 | 12.5 | 14.8 | 18.6 |
| Resolution range for refinement (Å) | | | | 20 to 2.0 | 20 to 1.8 | 50 to 2.25 |
| Number of protein atoms | | | | 6486 | 13236 | 13236 |
| Number of water molecules | | | | 1192 | 2016 | 1052 |
| Number of heterogen atoms | | | | 2 | 28 | 56 |
| Rmsd bound lengths (Å) | | | | 0.015 | 0.013 | 0.005 |
| Rmsd bond angles (°) | | | | 1.56 | 1.62 | 1.19 |
| Average B-factor (Å²) | | | | 30.6 | 25.7 | 26.6 |
| R_{work}²(%) | | | | 16.5 | 20.3 | 17.3 |
| R_{free}³ | | | | 19.1 | 24.7 | 21.7 |

¹R_{sym} = ΣₕΣⱼ|Iₕⱼ − <Iₕ>|/ΣₕΣⱼIₕⱼ, where Iₕⱼ is the intensity of observation j of reflection h.
²R_{work} = Σₕ∥F_{obs}| − |F_{calc}∥/Σ|F_{obs}|for all reflections above I/σ cutoff of 2.0, where F_{obs} and F_{calc} are observed and calculated structure factors, respectively.
³R_{free} was calculated against 10% of the complete data set excluded from refinement.

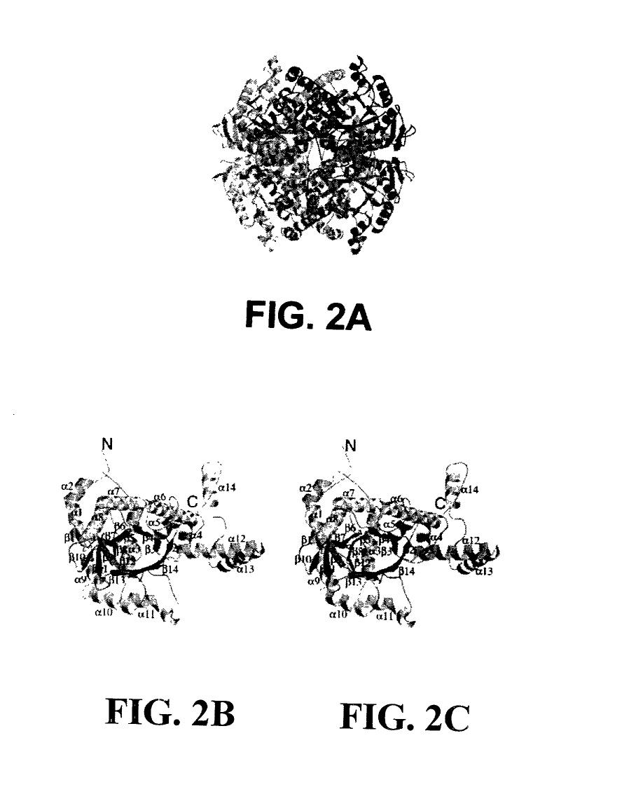

Overall Structure of ICL and Helix Swapping. ICL is a tetramer (81 Å×86 Å×92 Å) with 222 symmetry. Each subunit of the enzyme is composed of 14 α-helices and 14 β-strands (FIG. 2A, FIG. 2B and FIG. 2C). Eight α-helices (α4-α11) and eight β-strands (β2-β5, β8, β12-β14), representing the largest domain and the core of the structure, form an unusual α/β-barrel. The α/β-barrel has a topology of (βα)₂α(βα)₅β, differing from the canonical (βα)₈ pattern. An additional helix, present after the eighth β-strand (helix α12, composed of residues 349-367) projects away from the barrel and, together with the two ensuing helices α13 (residues 370-384) and α14 (residues 399-409), forms interactions exclusively with the neighboring subunit.

Residues 184-200 and 235-254 (connecting the third and fourth β-strands to their consecutive helices, respectively) form a small β-domain consisting of a short five-stranded β-sheet (β6, β7, β9, β10, β11) which lies atop the α/β-barrel. This domain is important as it contains several of the active site residues. A long insertion of about 100-160 amino acids in this β-sheet starting near residue Glu 247 is found in several ICL sequences. This extra domain, observed mostly in plant ICLs, has been proposed to be responsible for targeting to peroxisomes (Matsuoka and McFadden, 1988).

The two noncrystallographically related subunits are interlinked by the exchange of the C-terminal region (FIG. 2A), which contain helices α12 and α13. Similar helix-swapping has been proposed to be an effective way of forming stable dimmers (Bennett et al., 1995). The interface between the two subunits involved in helix swapping buries ˜18% of the accessible surface of each subunit. In ICL, knotting of the polypeptide chains suggests that the formation of the two subunit complex is concomitant with the folding of the subunits. Of the structures available in the Protein Data Bank, ICL also shows some resemblance to phosphoenolpyruvate mutase (accession code 1PYM), which is a smaller protein (295 residues) and lacks the N-terminal domain, three strands of the small β-domain and part of the C-terminal domain, but shows similar helix-swapping.

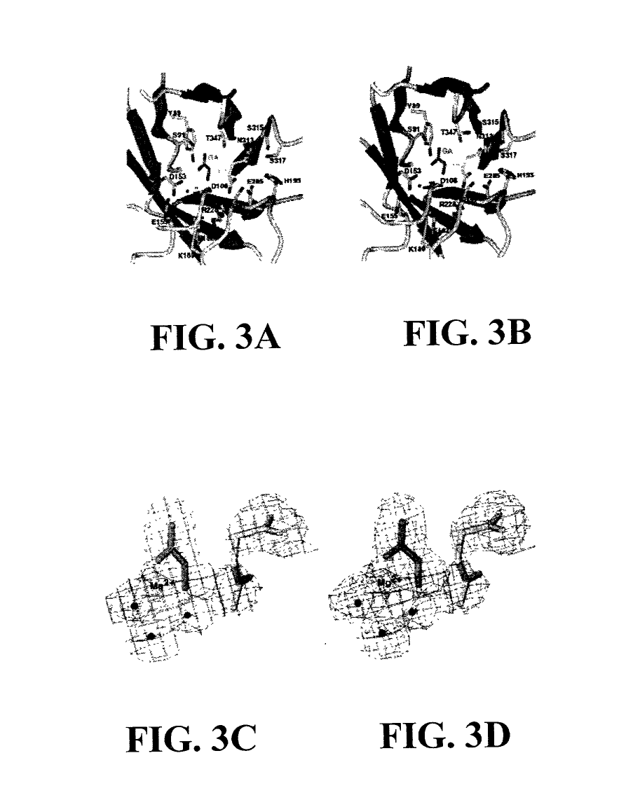

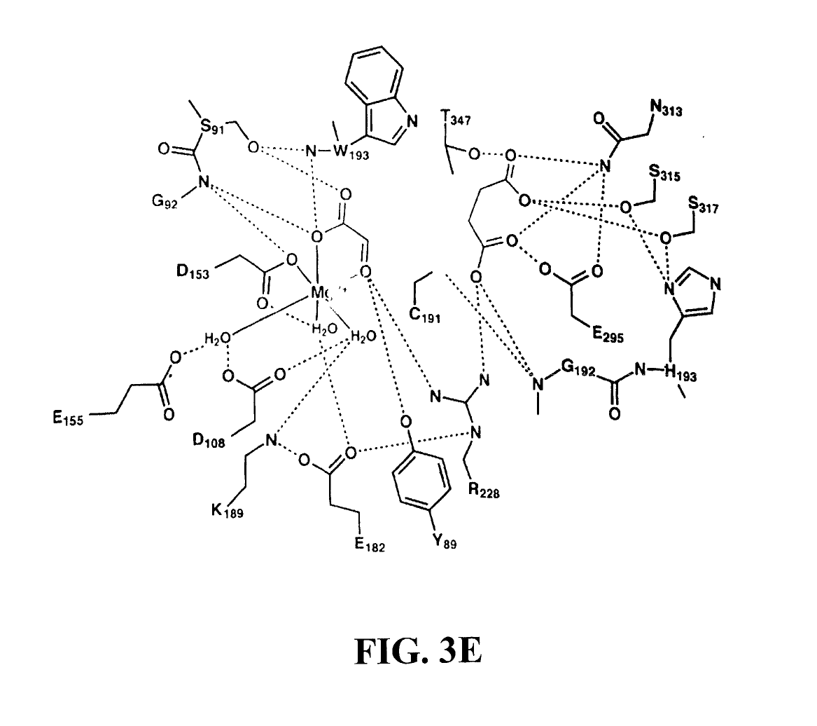

3. Binding of 3-Nitropropionate and 3-Bromopyruvate. The ternary complex with glyoxylate and the nonreactive succinate analog 3-nitropropionate was useful in elucidating mechanistic information from ICL. Stable binding of both nitropropionate and glyoxylate was achieved by carrying out the structure determination of a mutant ICL in which the active site nucleophile Cys 191 was changed to Ser. As is seen typically in α/β barrels, the active site of ICL is located at the C-terminal ends of the β-strands (FIG. 3A and FIG. 3B). Clear density was observed for glyoxylate, 3-nitropropionate and Mg²⁺ in noncrystallographically symmetry (NCS) averaged difference maps contoured at 2σ level (FIG. 3C and FIG. 3D). The planar glyoxylate binds by coordination to the active site Mg²⁺ ion and forms hydrogen bonds with the residues Ser 91 OG, Gly 92 N, Trp 93 N and Arg 228 NH2 of the protein (FIG. 3E).

As the nitro and carboxylate groups of 3-nitropropionate were not distinguished, a succinate molecule was fit to the density such that one carboxylate makes specific hydrogen bonds with the side chains of residues Asn 313 ND1, Glu 295 OE2, Arg 228 NH1 and Gly 192 N, while the second carboxylate forms hydrogen bonds with Thr 347 OG, Asn 313 ND2, Ser 315 OG, Ser 317 OG and His 193 ND1. The protein surface that packs against the C2 and C3 methylene carbons of succinate is provided by residues Trp 93, Thr 347 and Leu 348. In the resulting orientation, the C2 carbon of succinate is located 3.9 Å from the aldehyde carbon of glyoxylate and 3.2 Å from the hydroxyl of Ser 191 of the C191 S mutant.

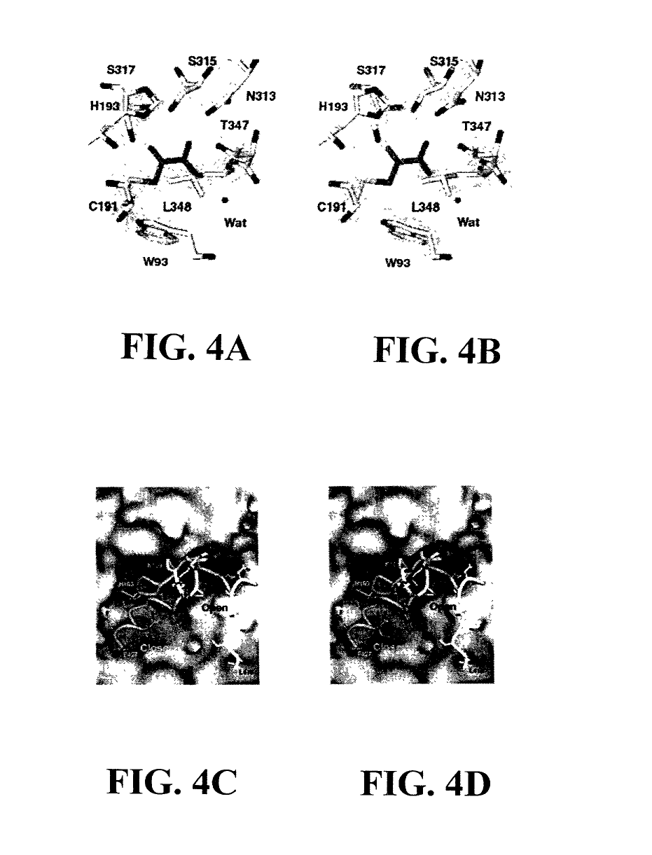

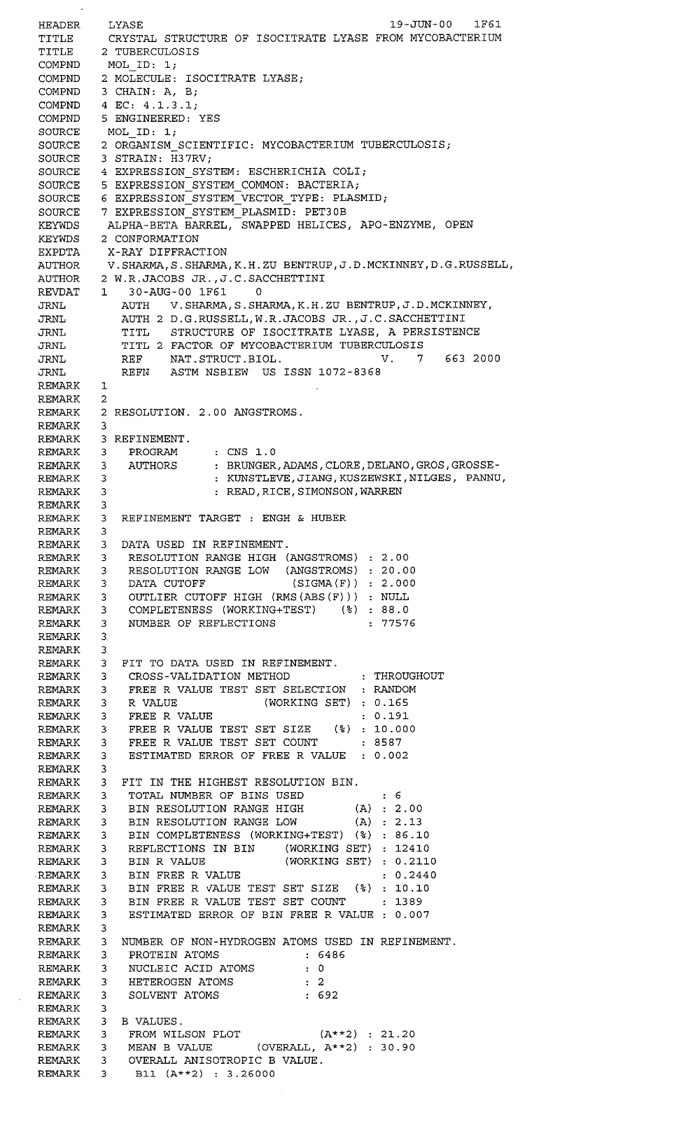

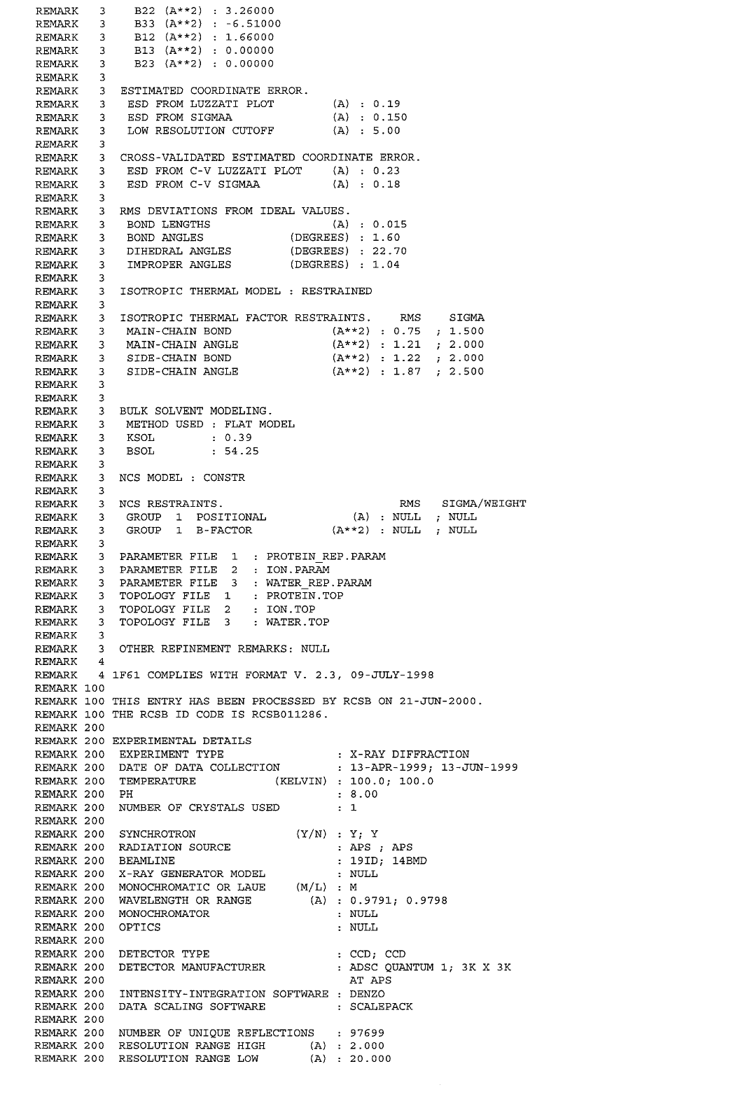

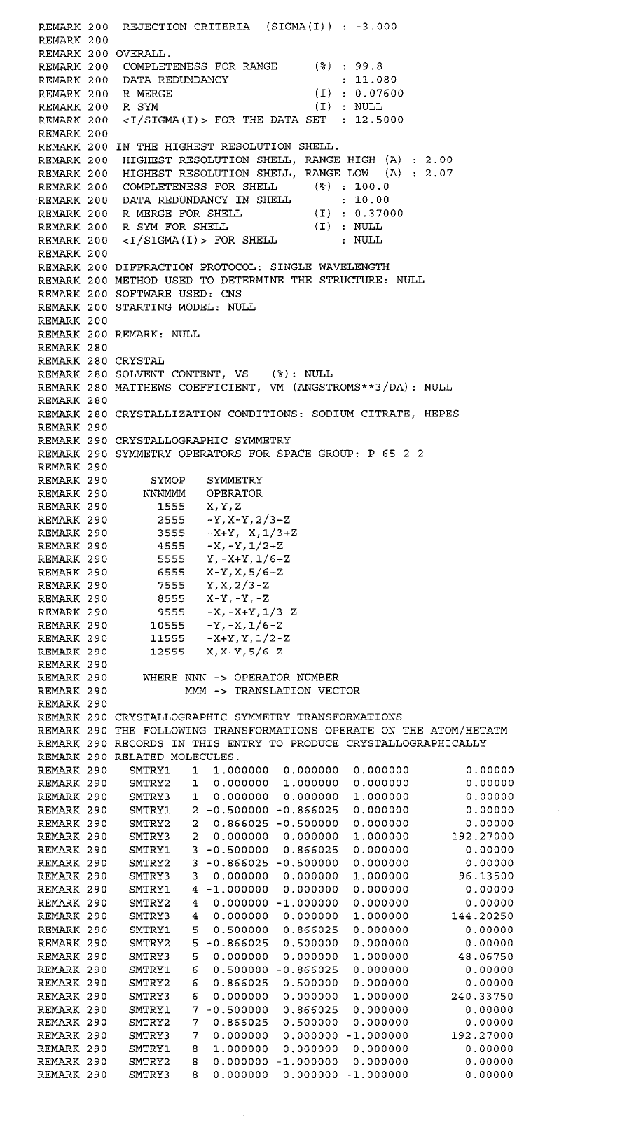

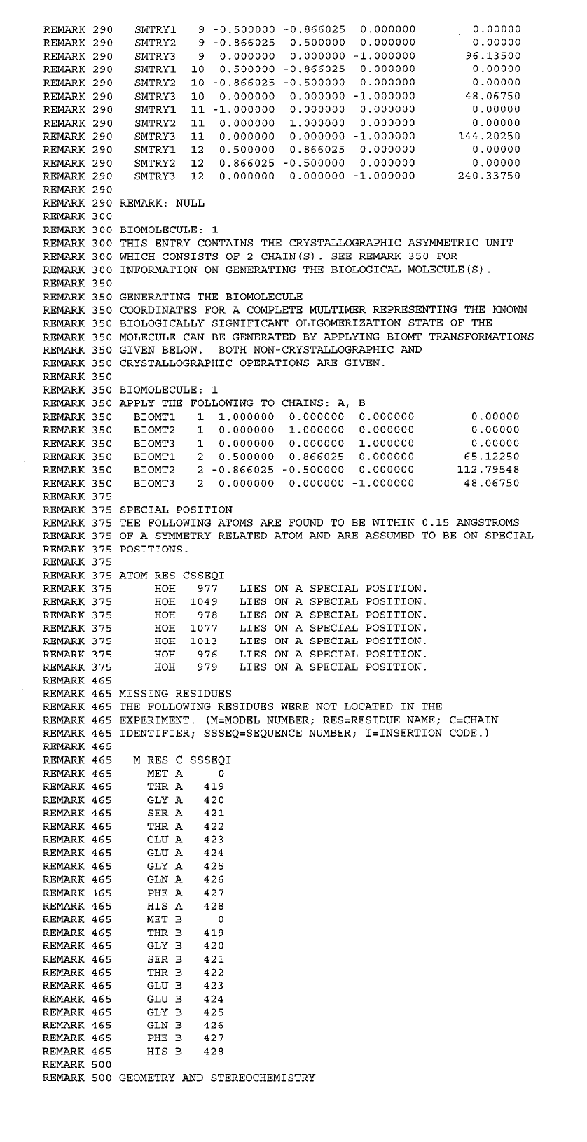

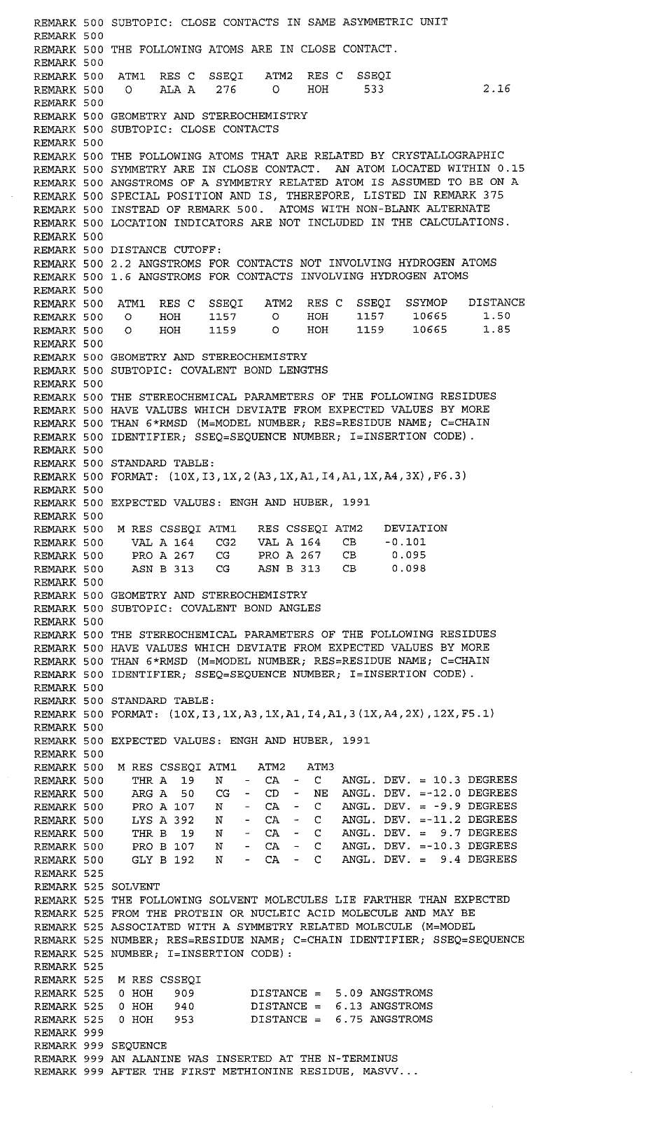

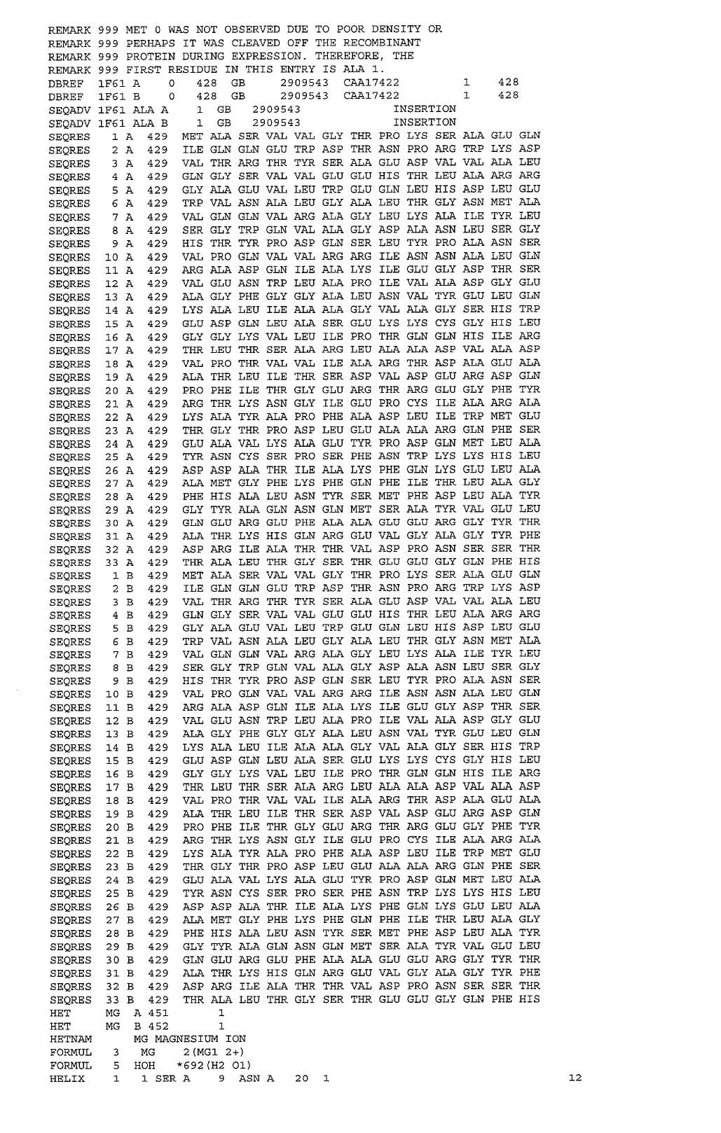

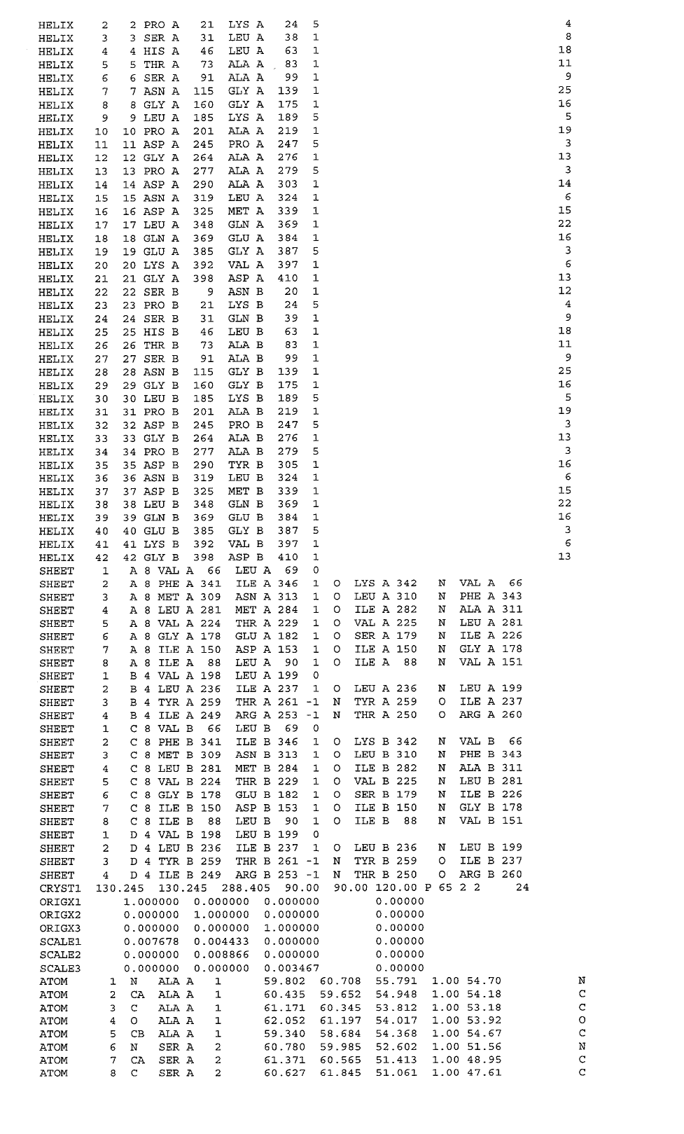

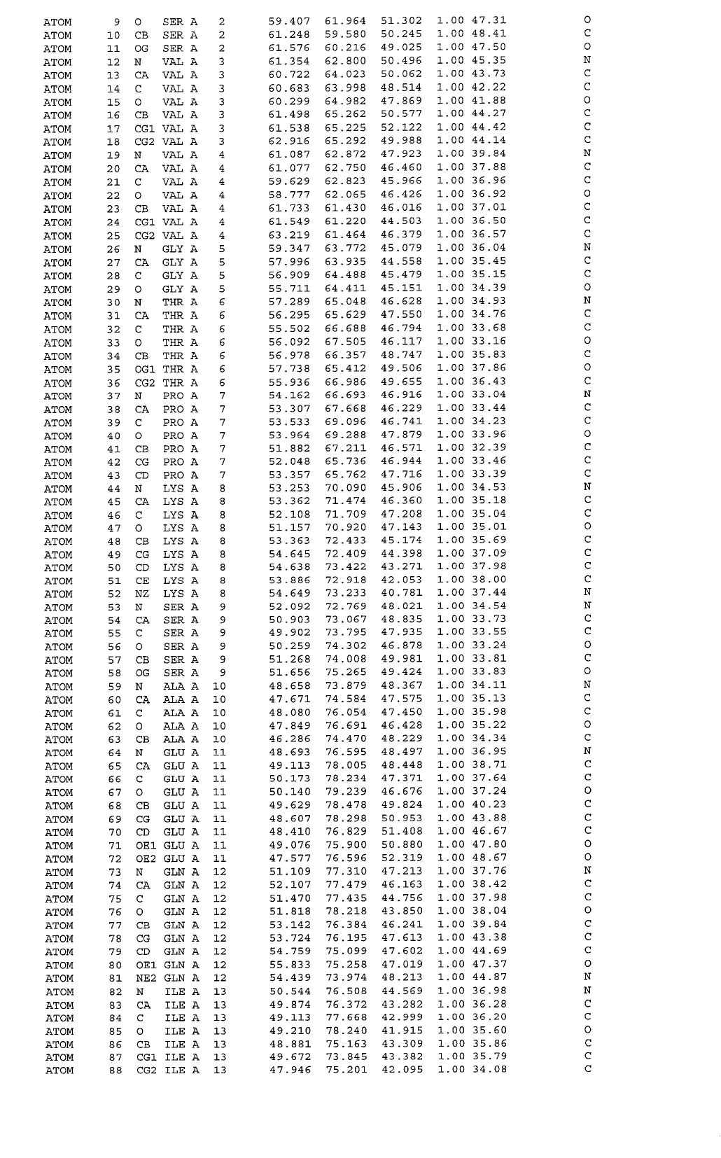

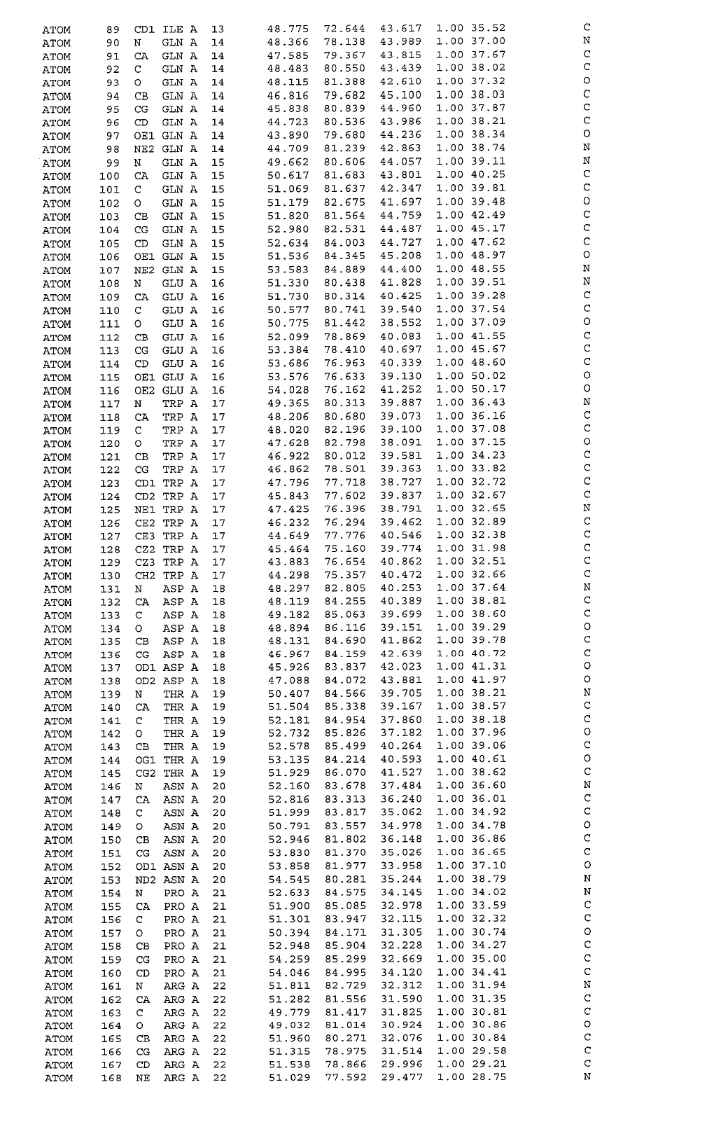

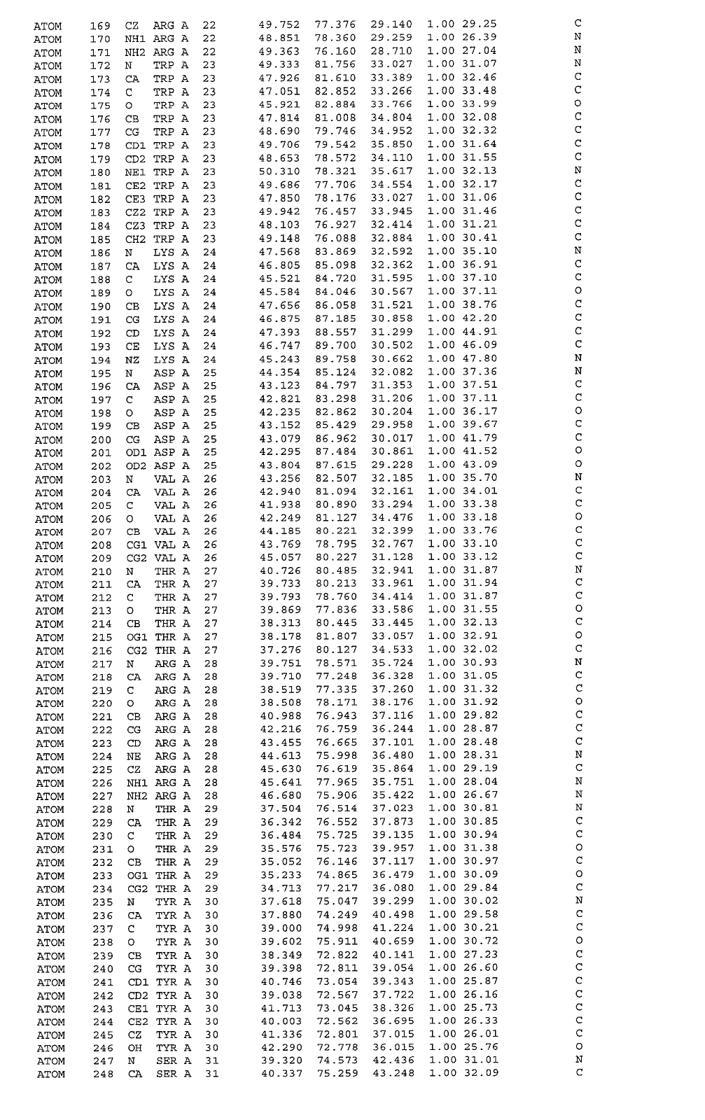

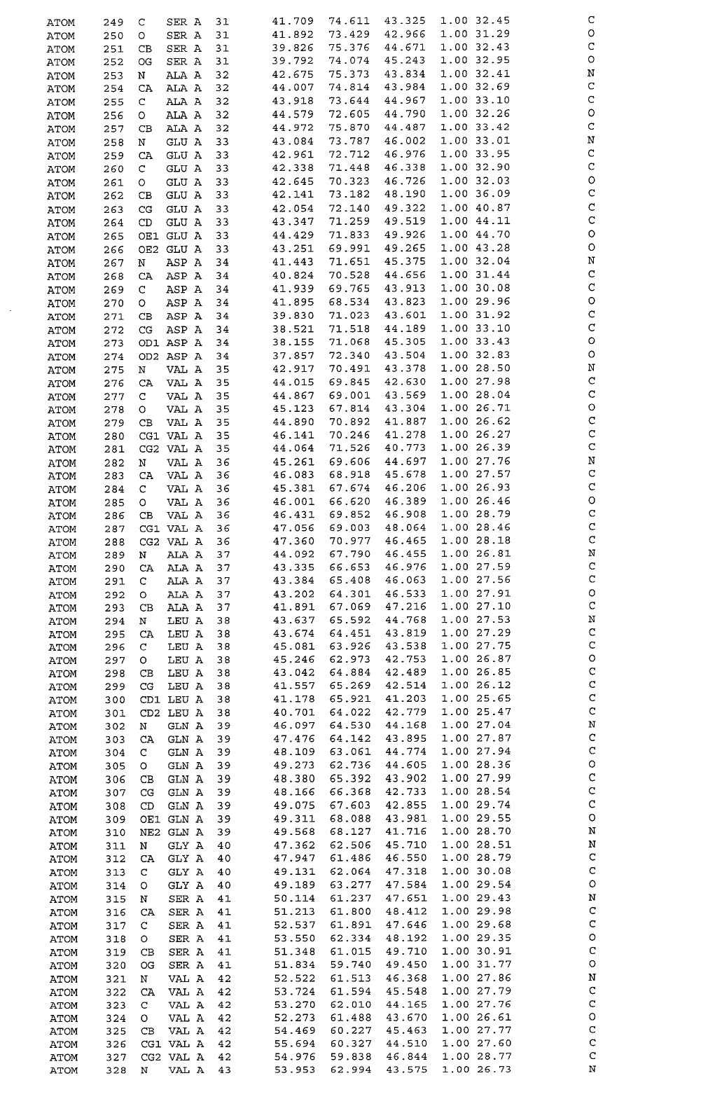

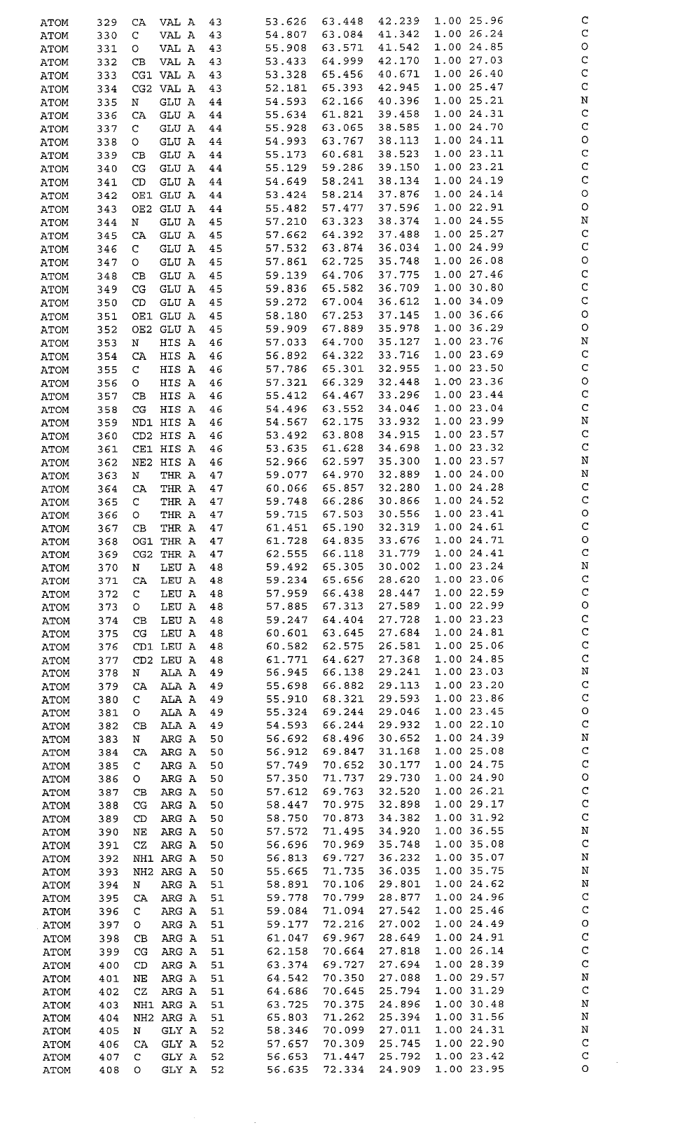

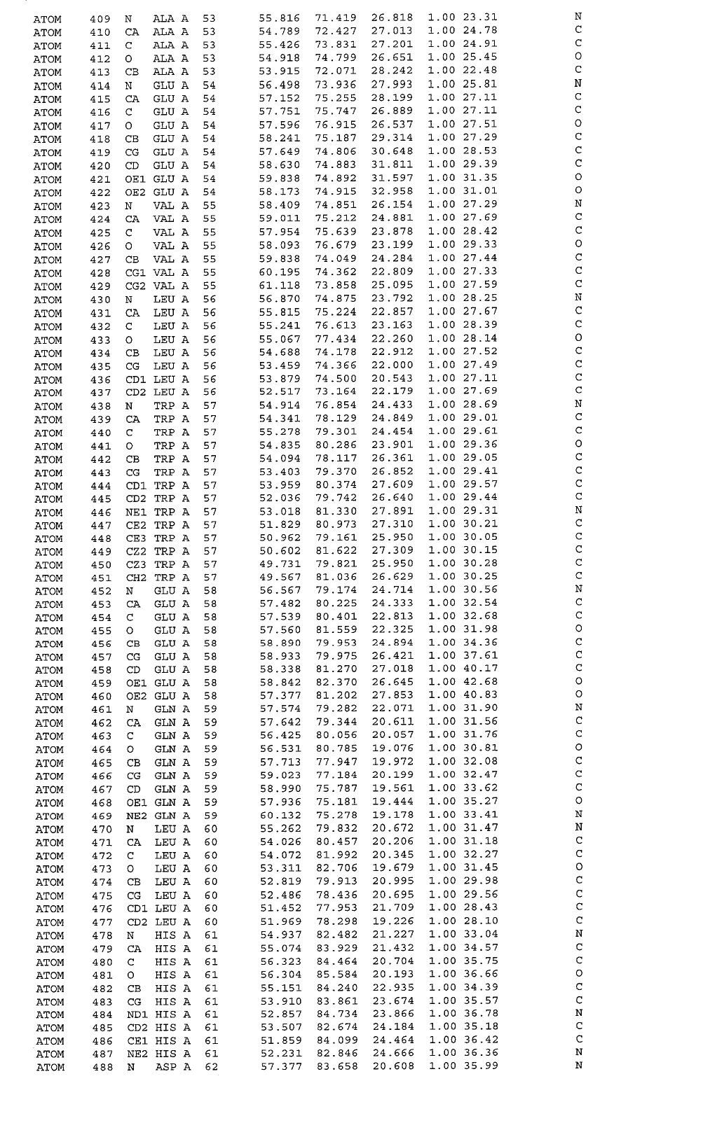

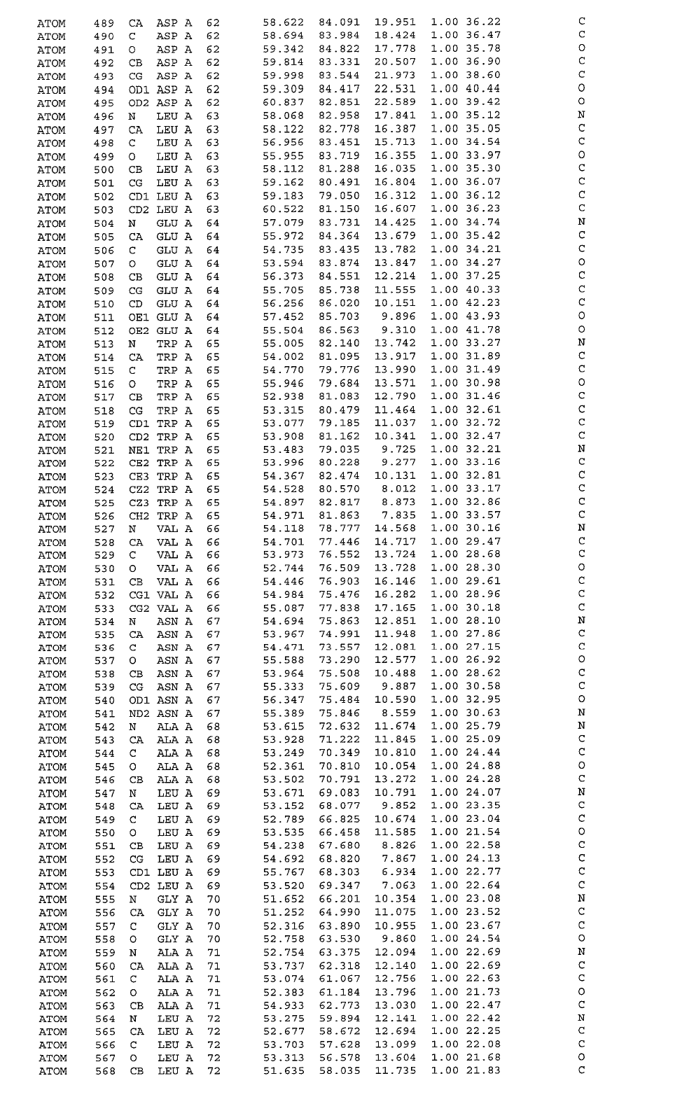

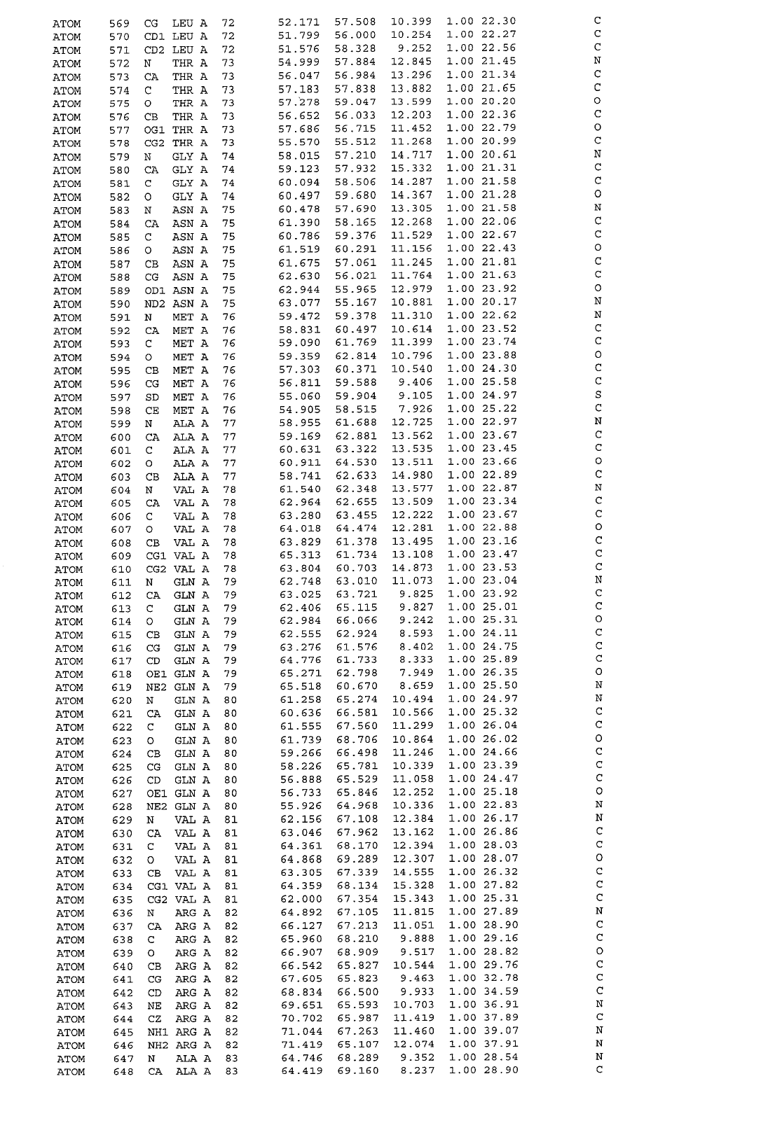

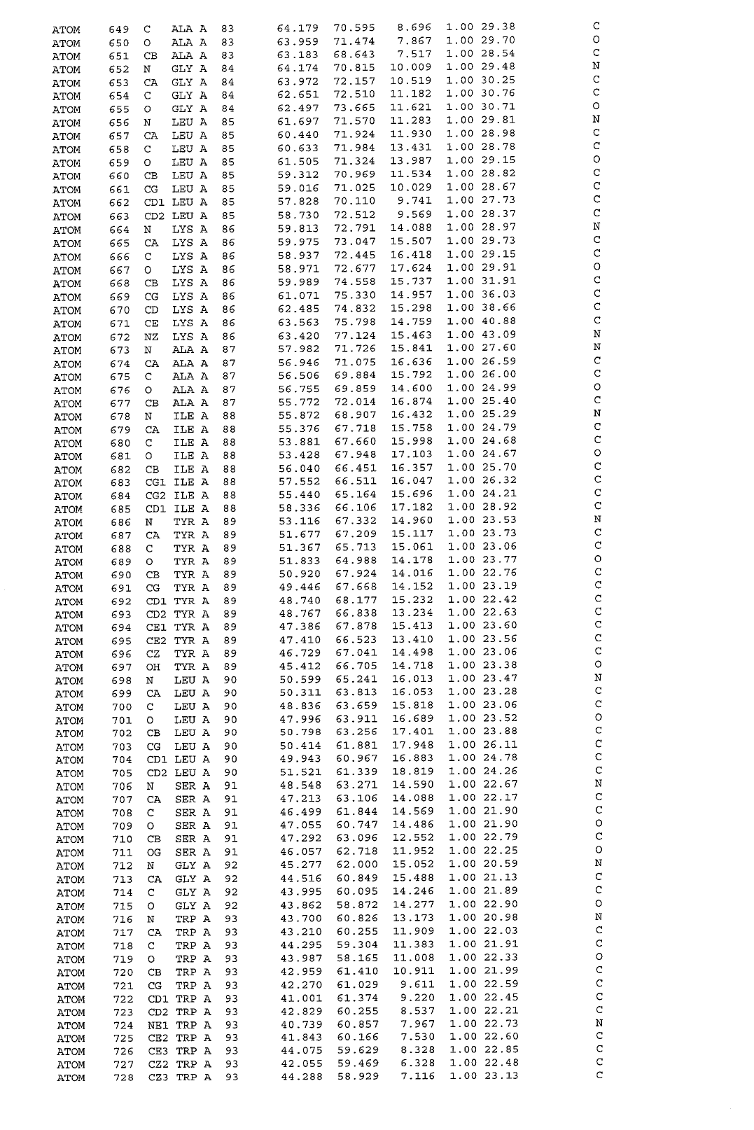

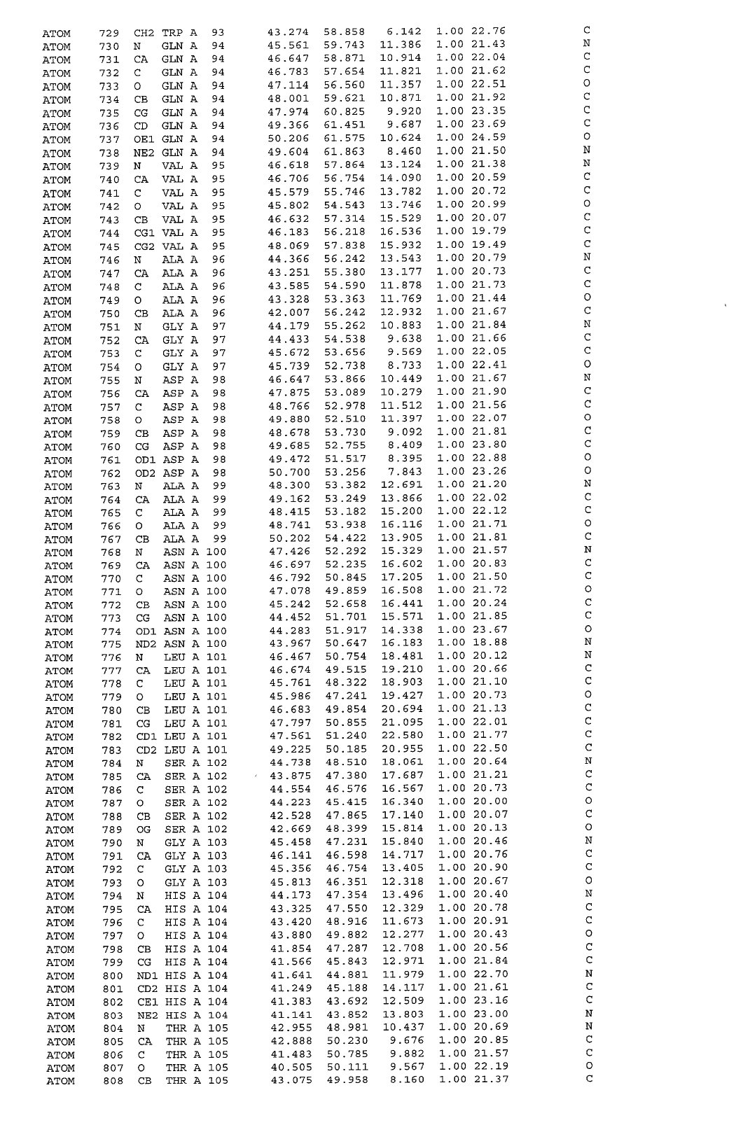

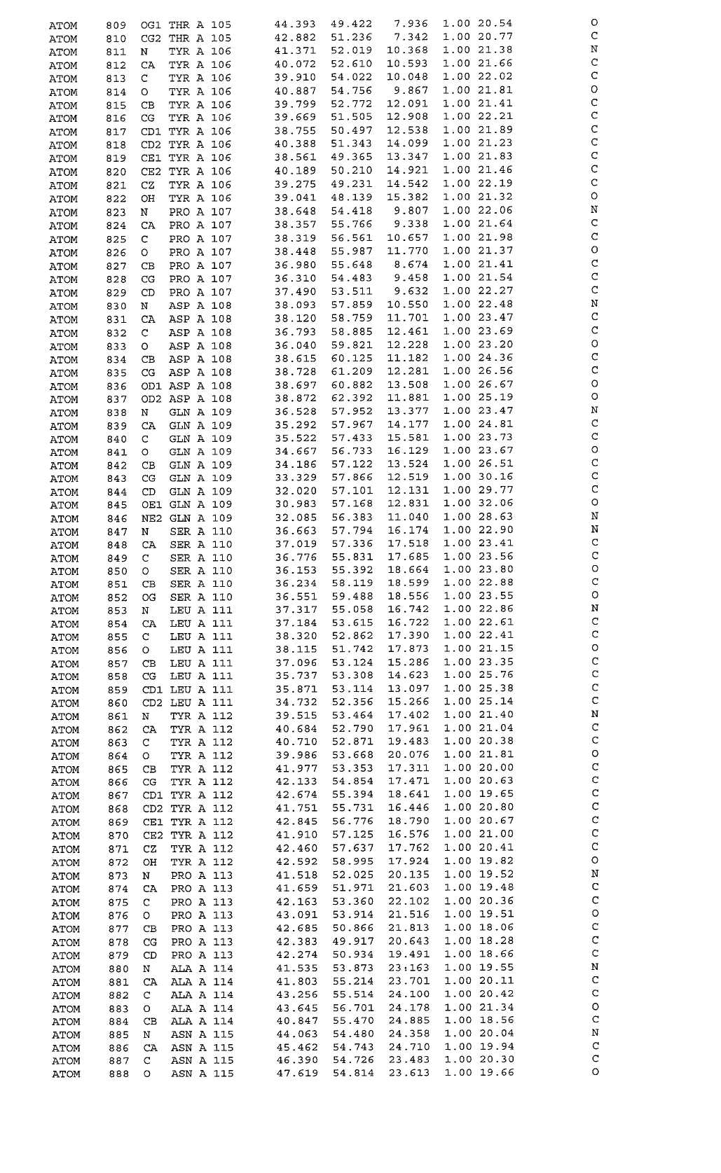

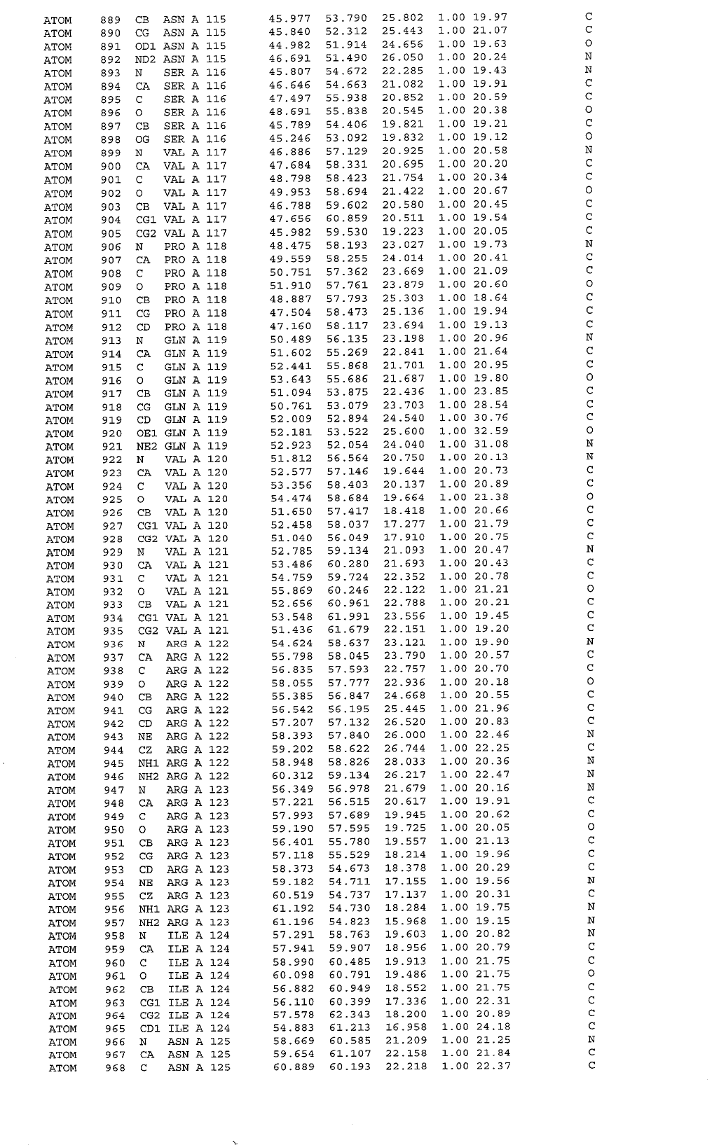

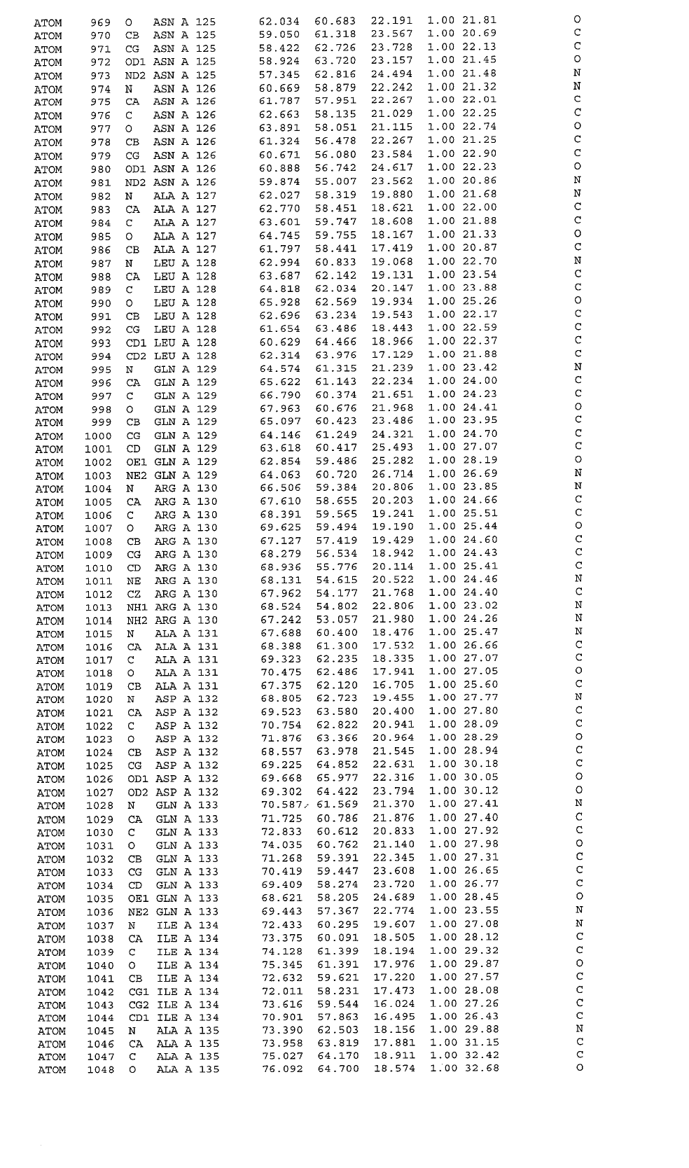

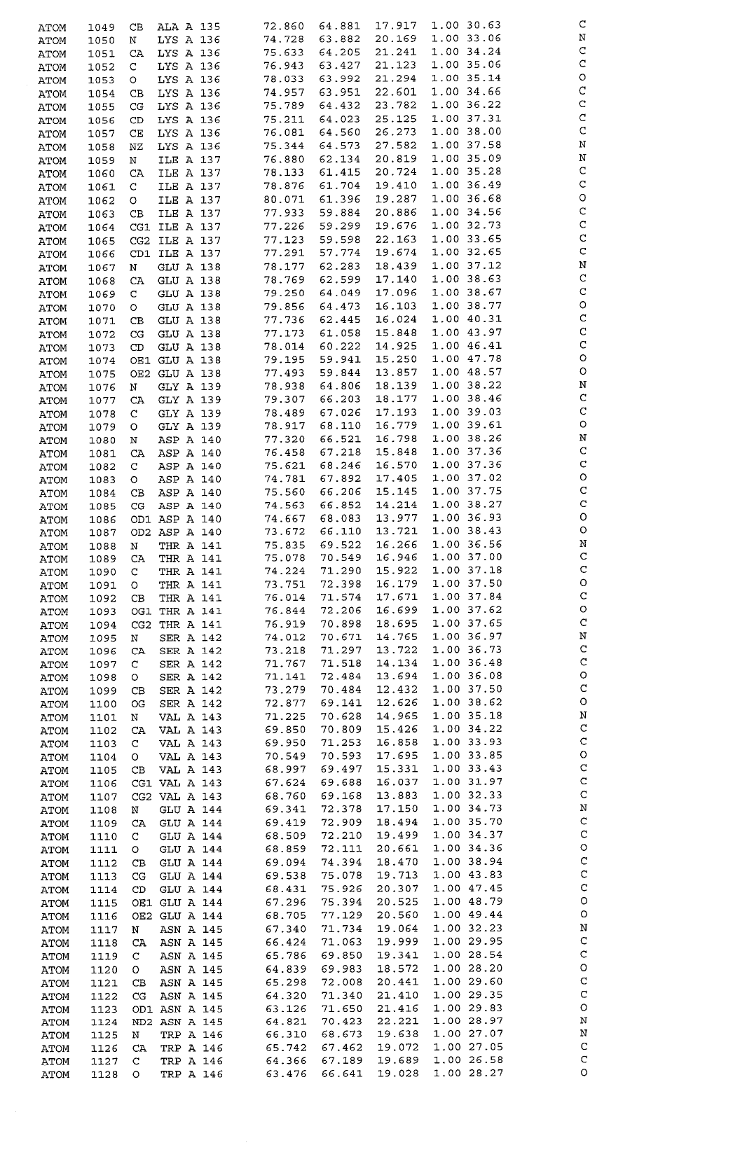

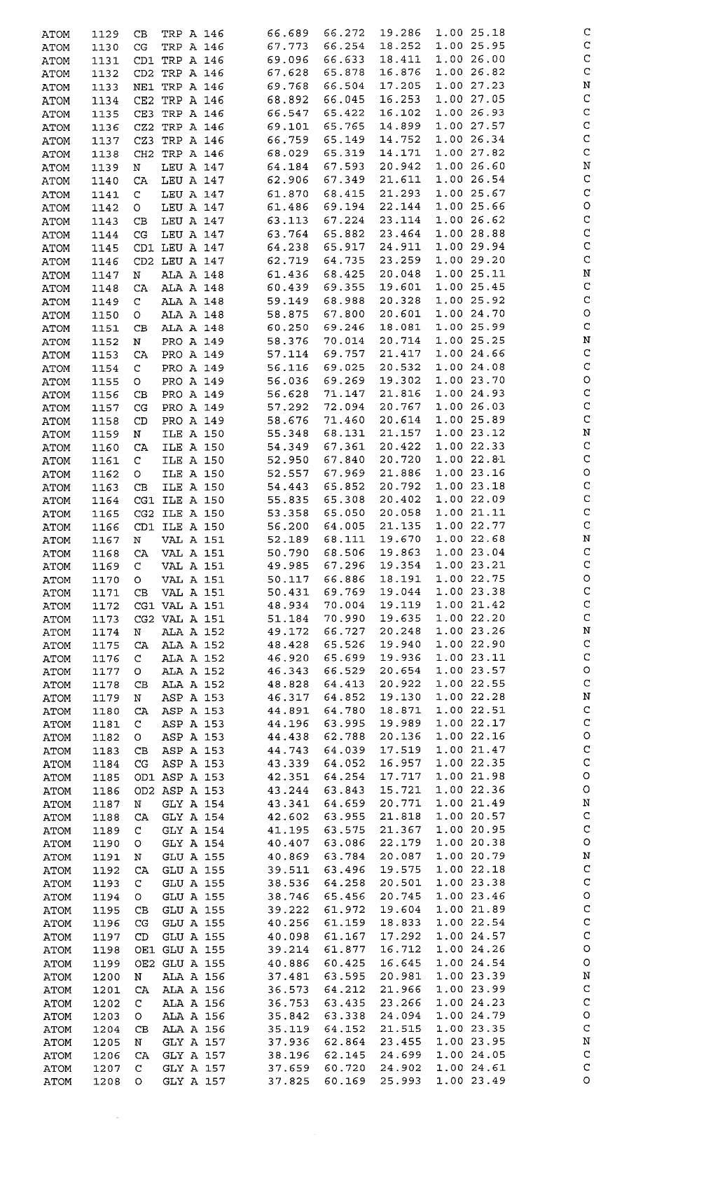

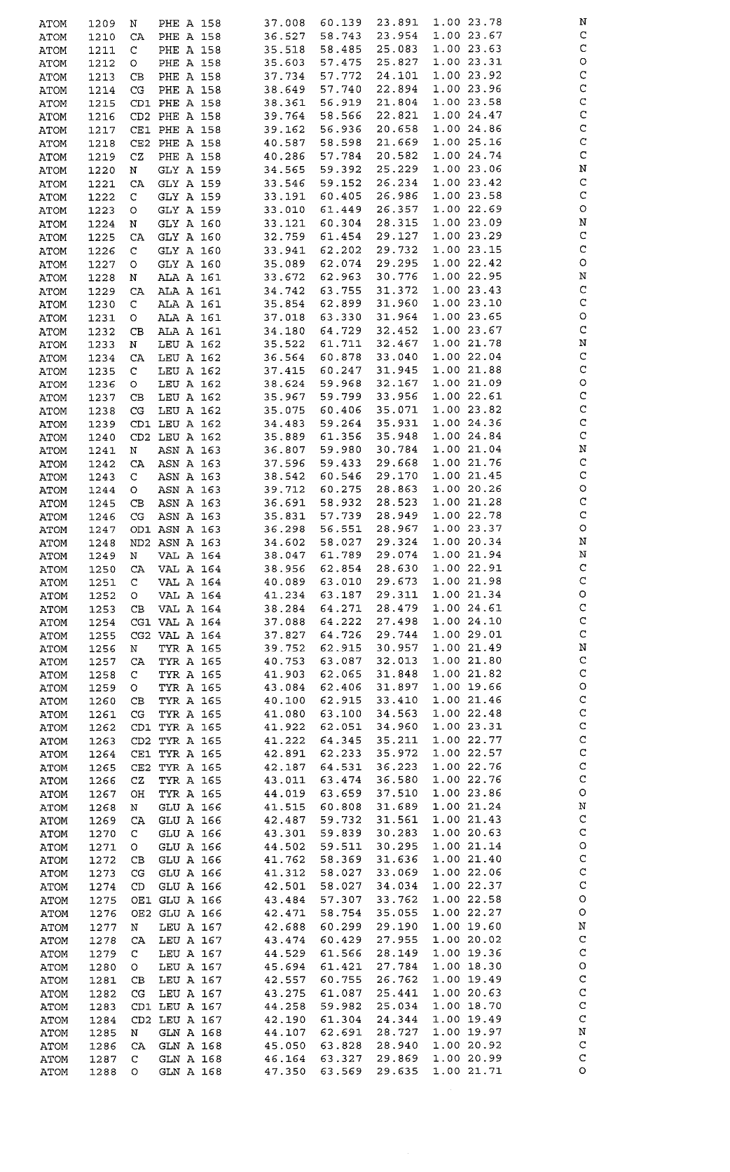

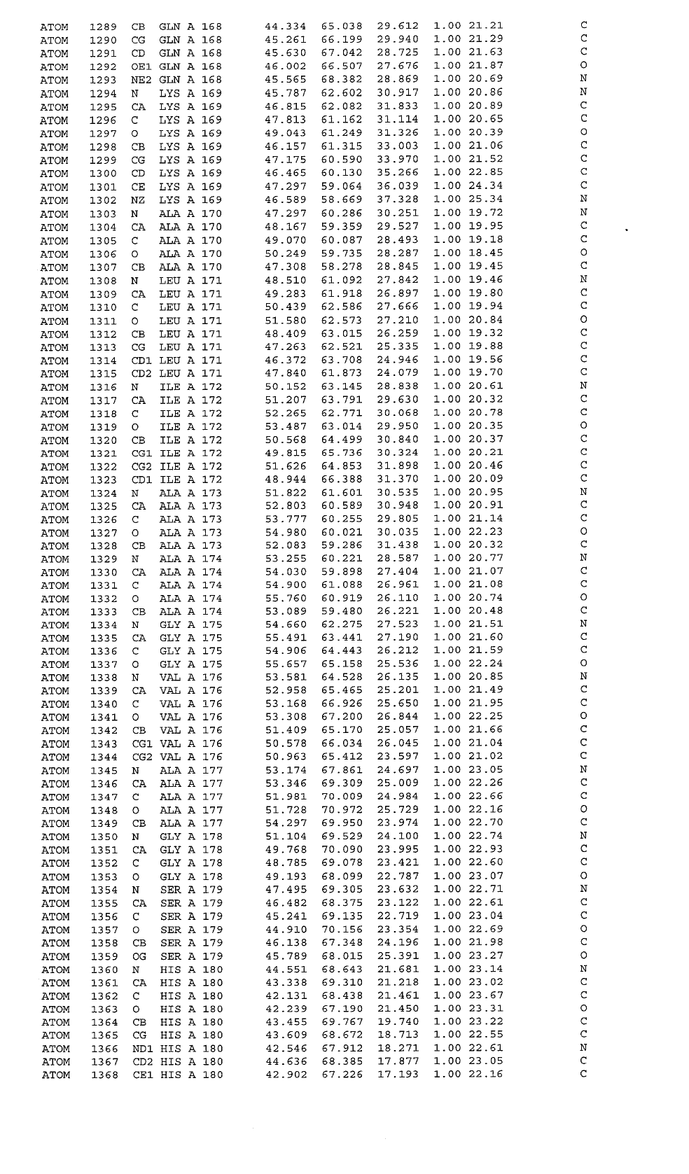

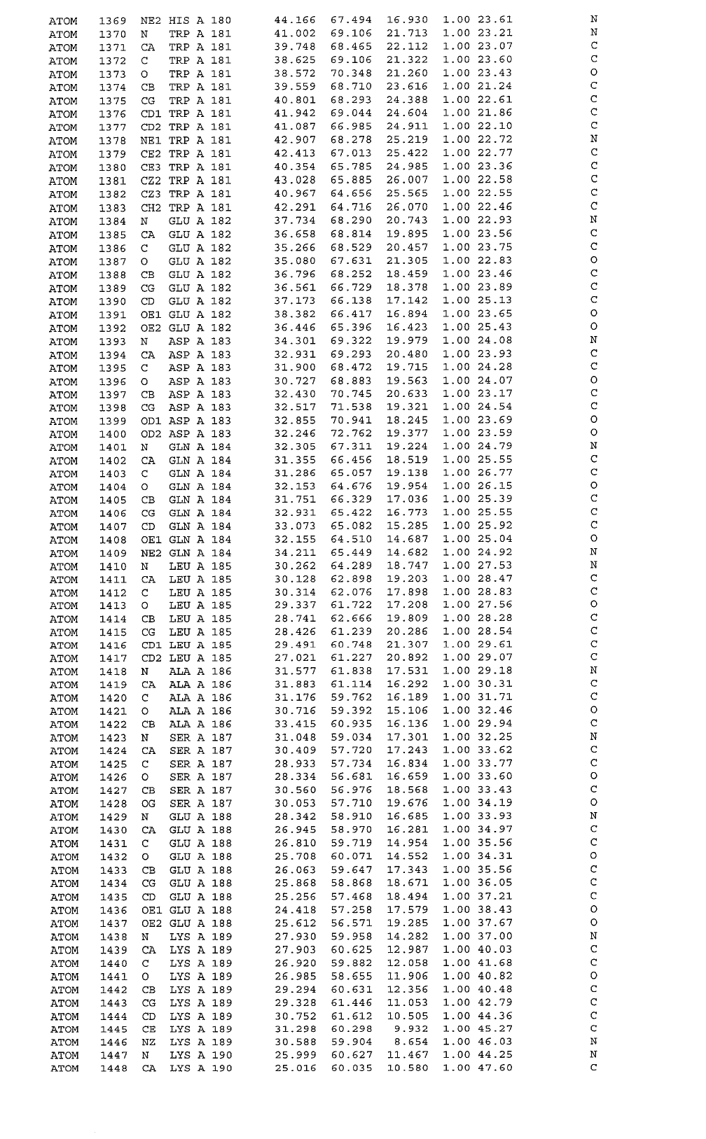

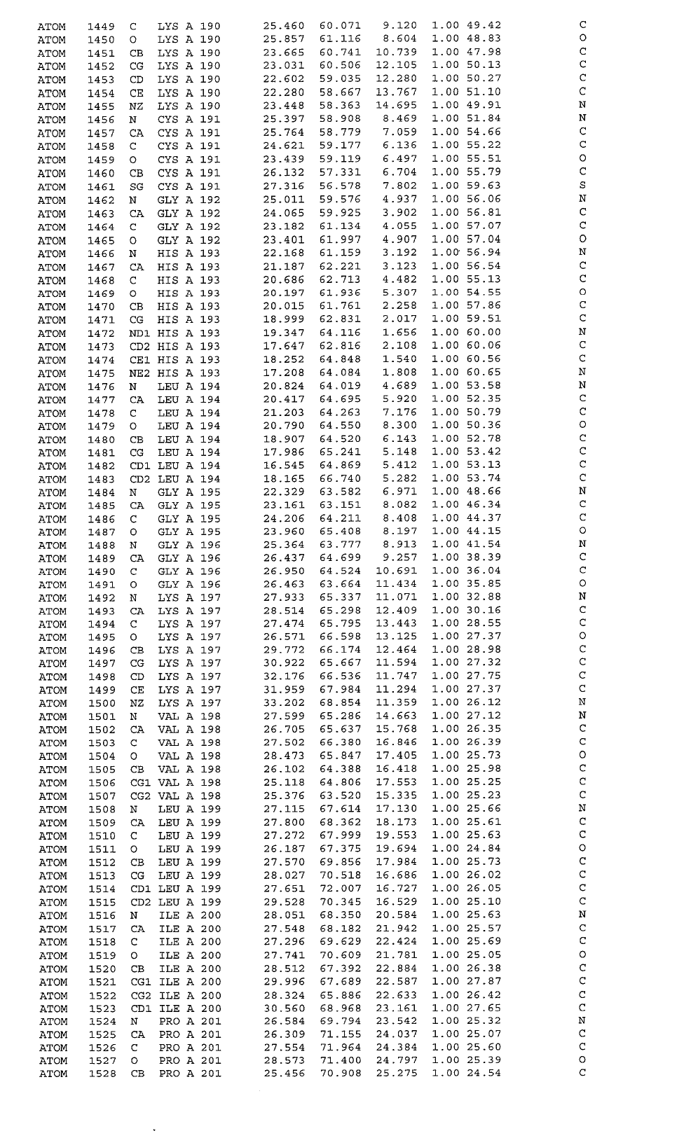

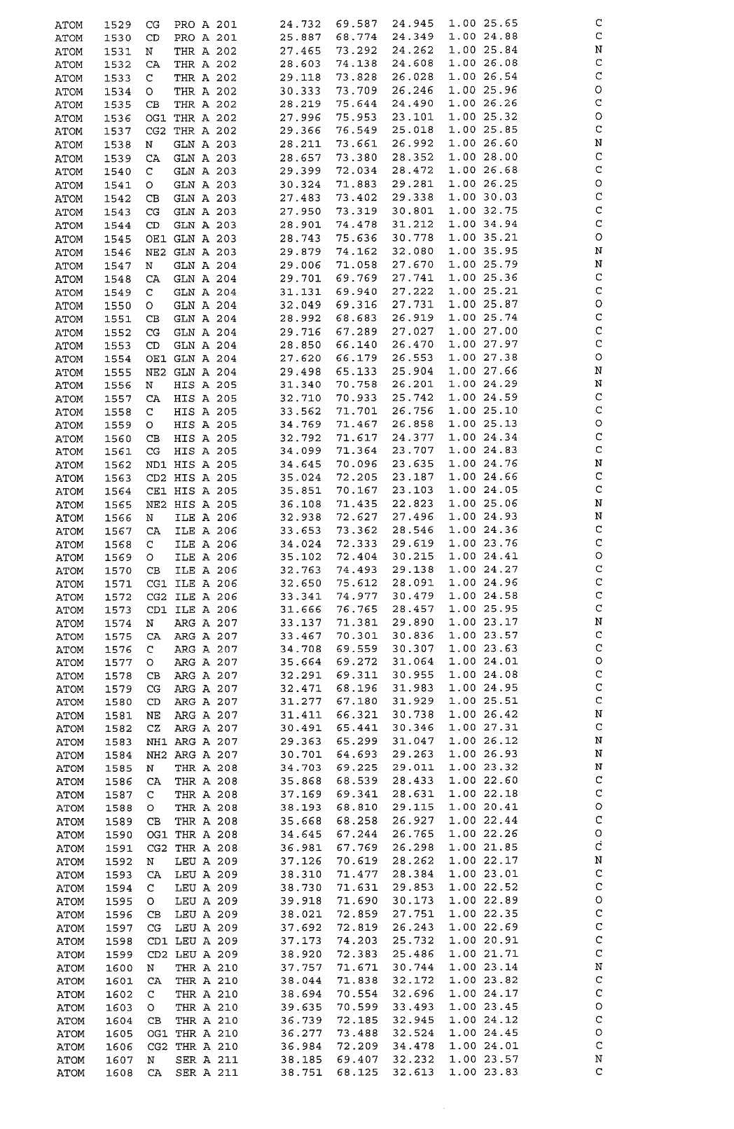

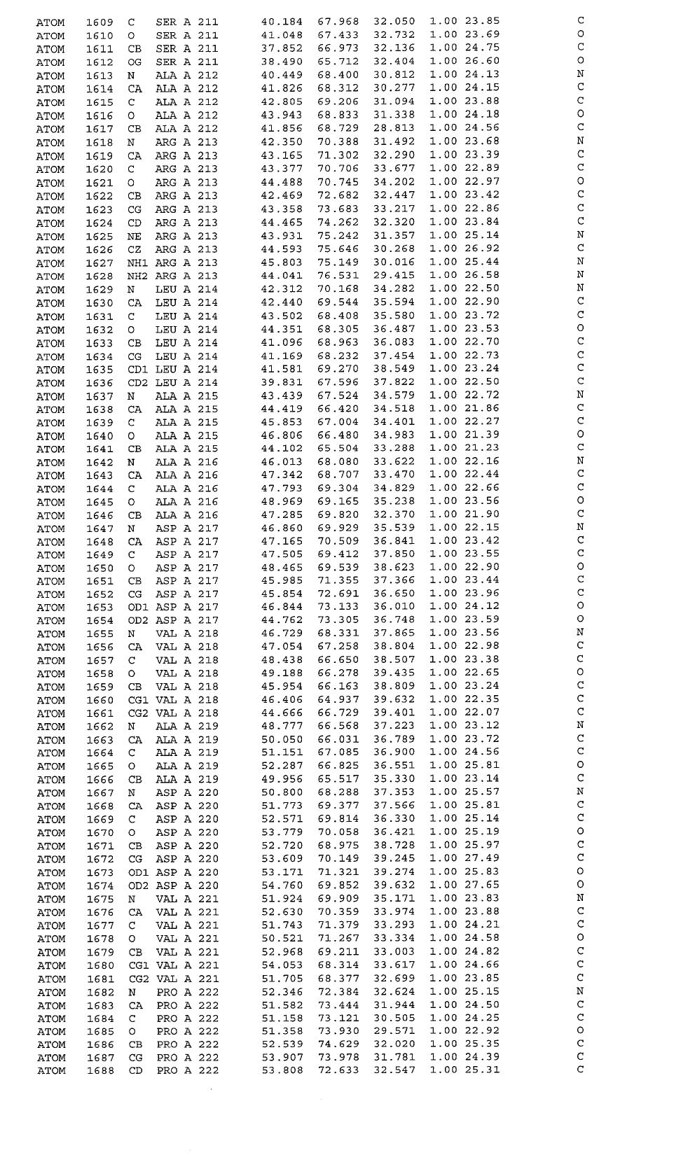

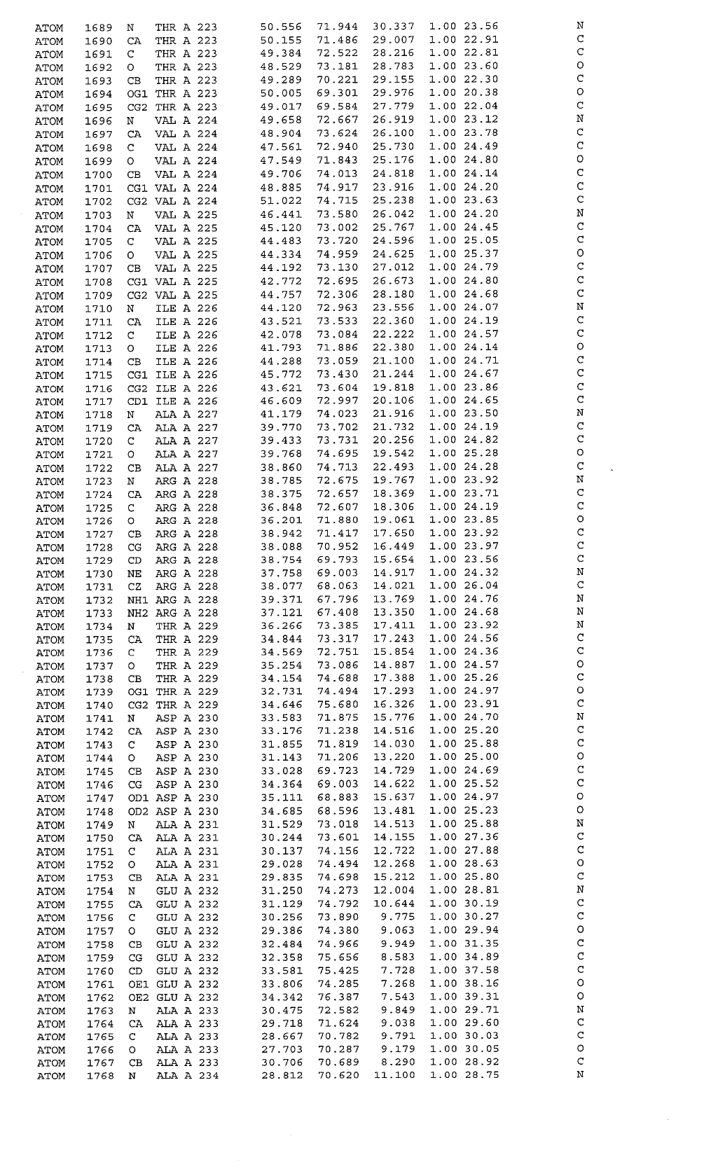

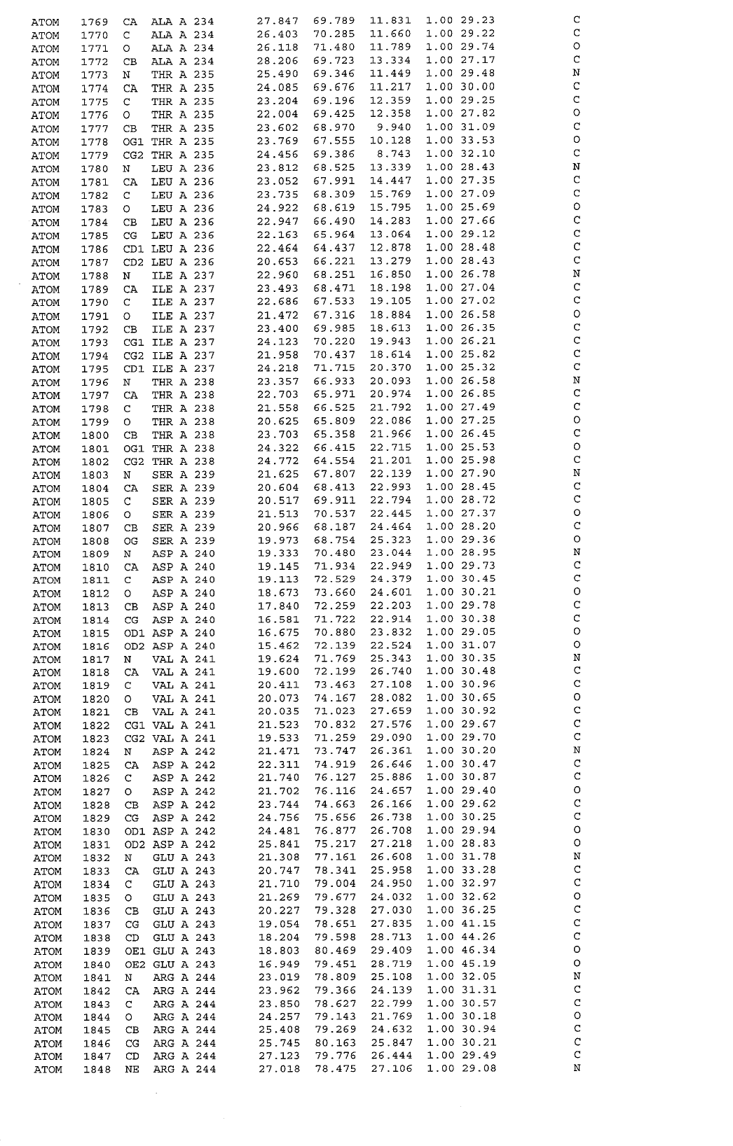

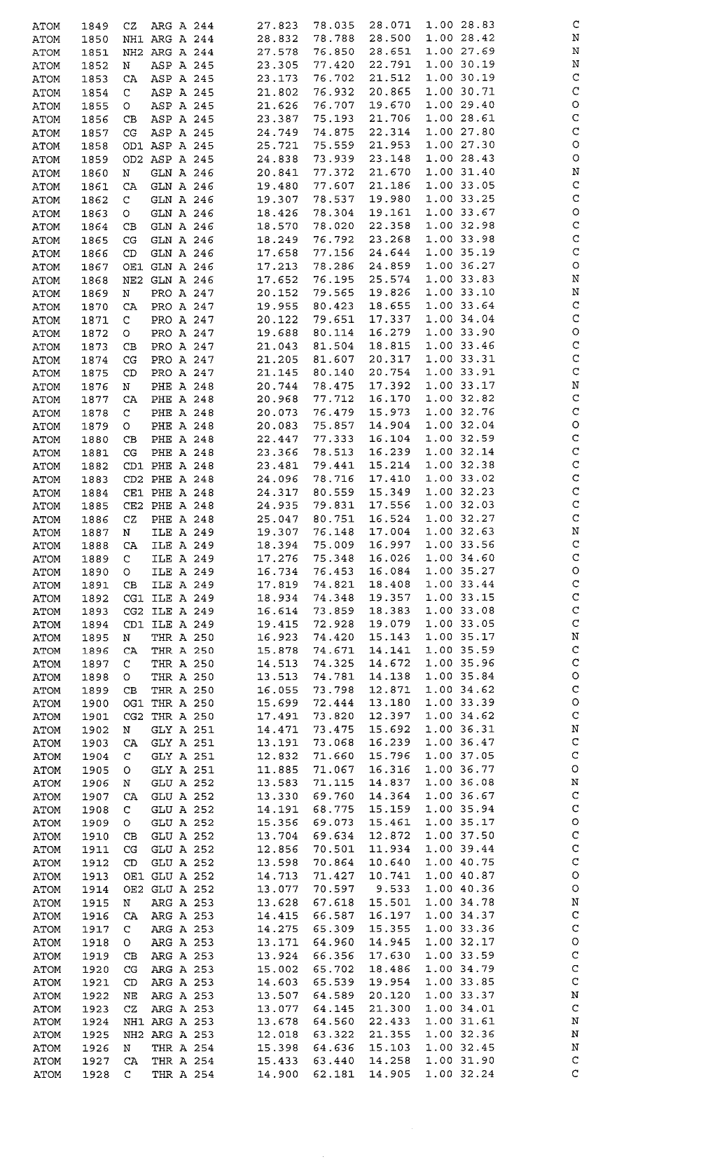

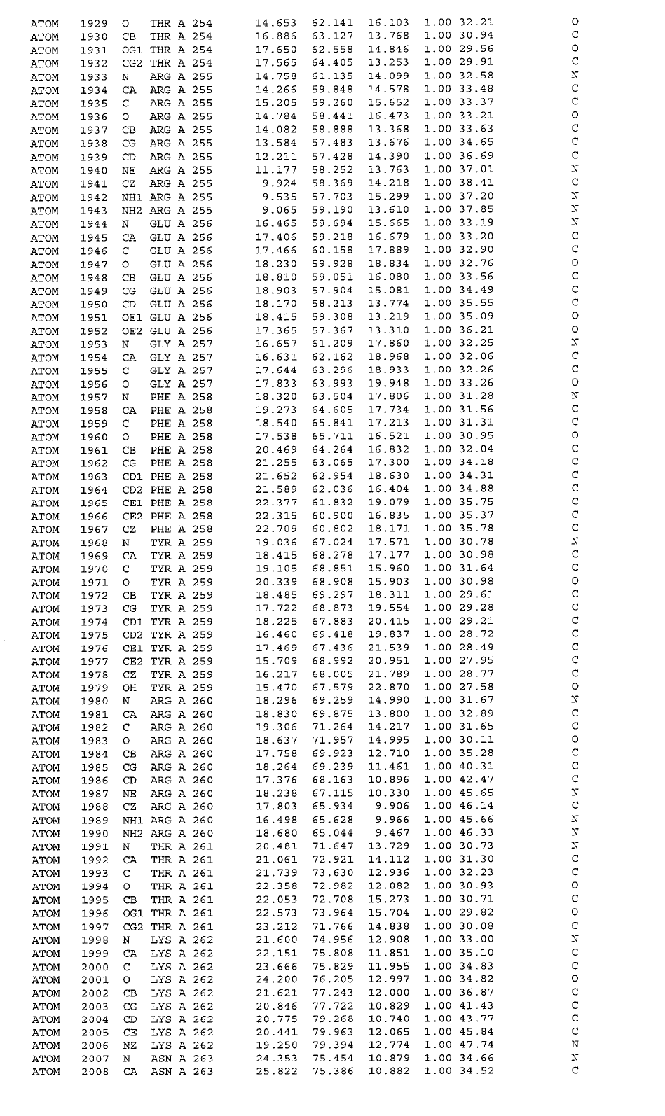

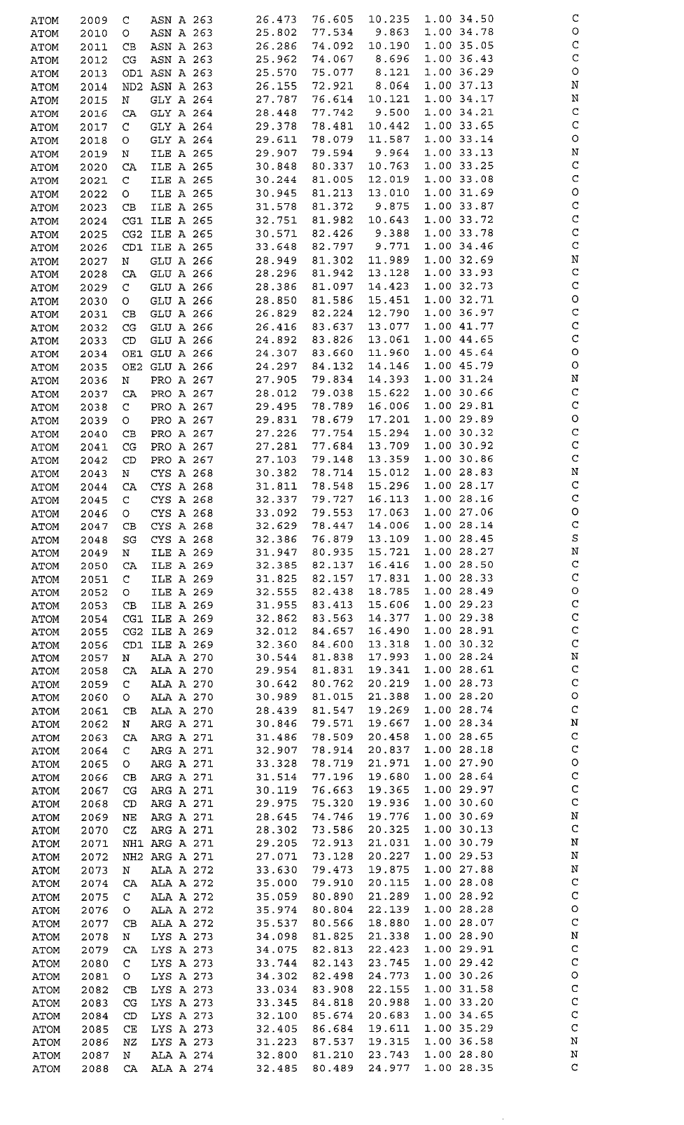

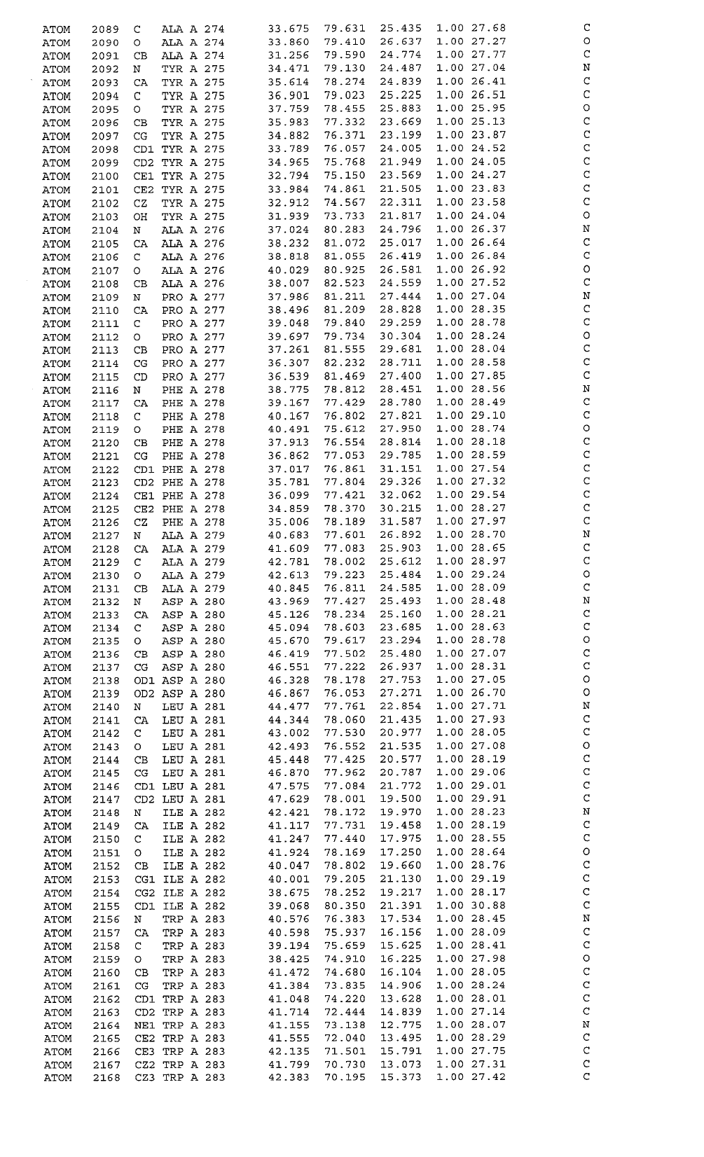

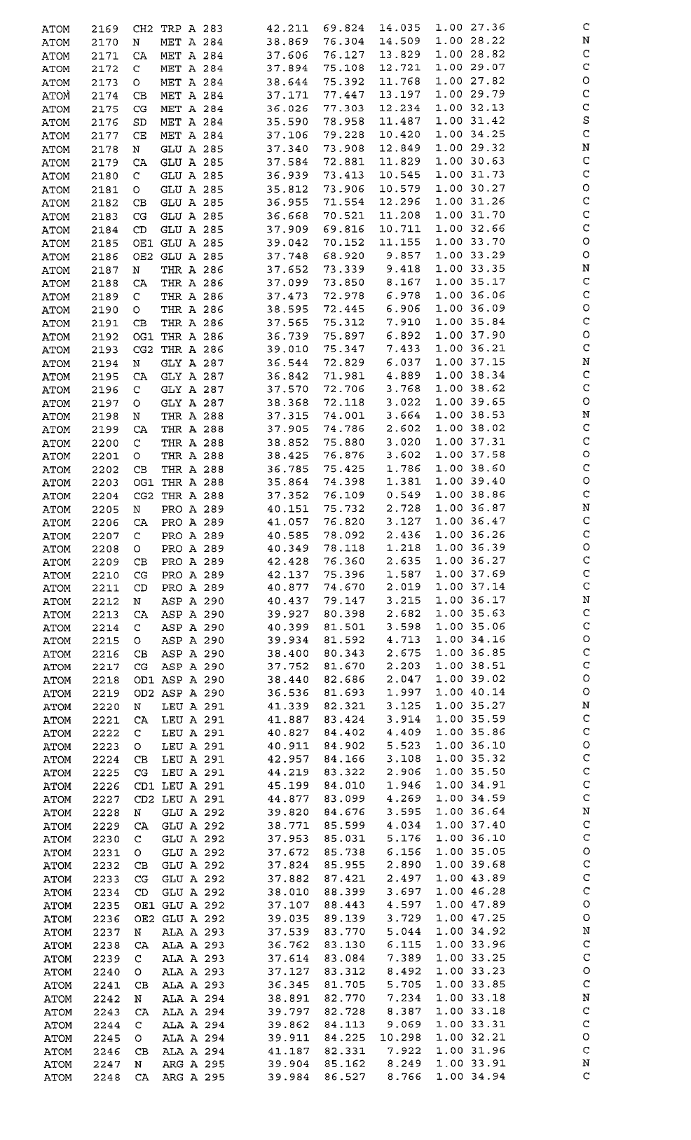

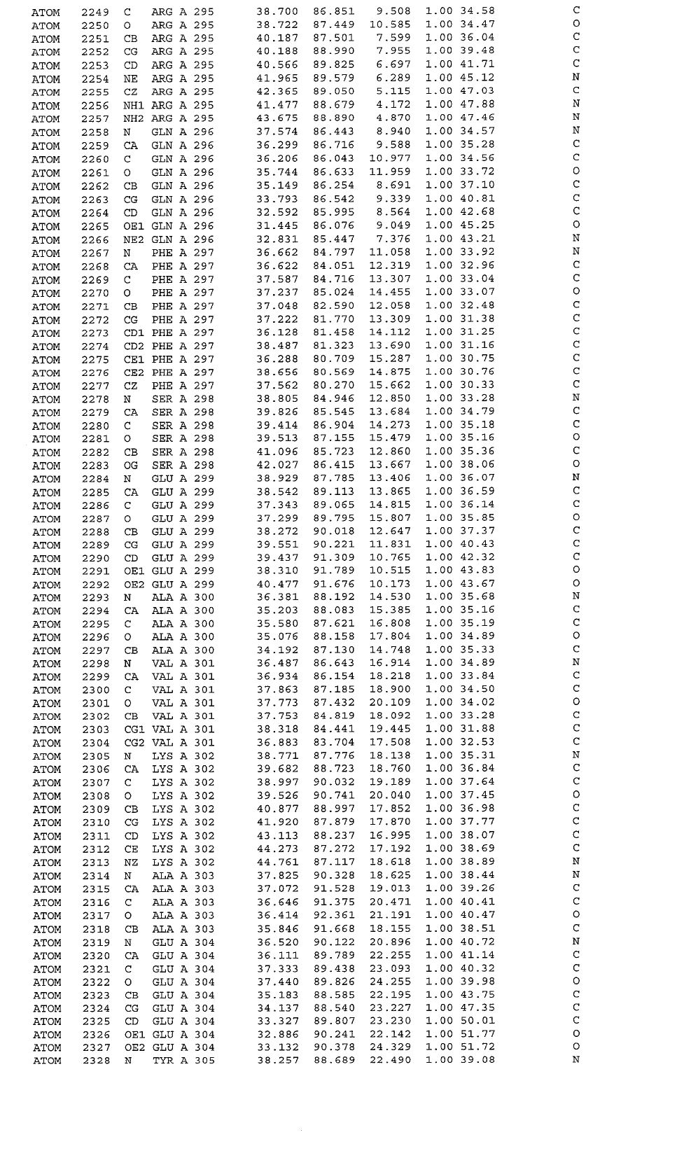

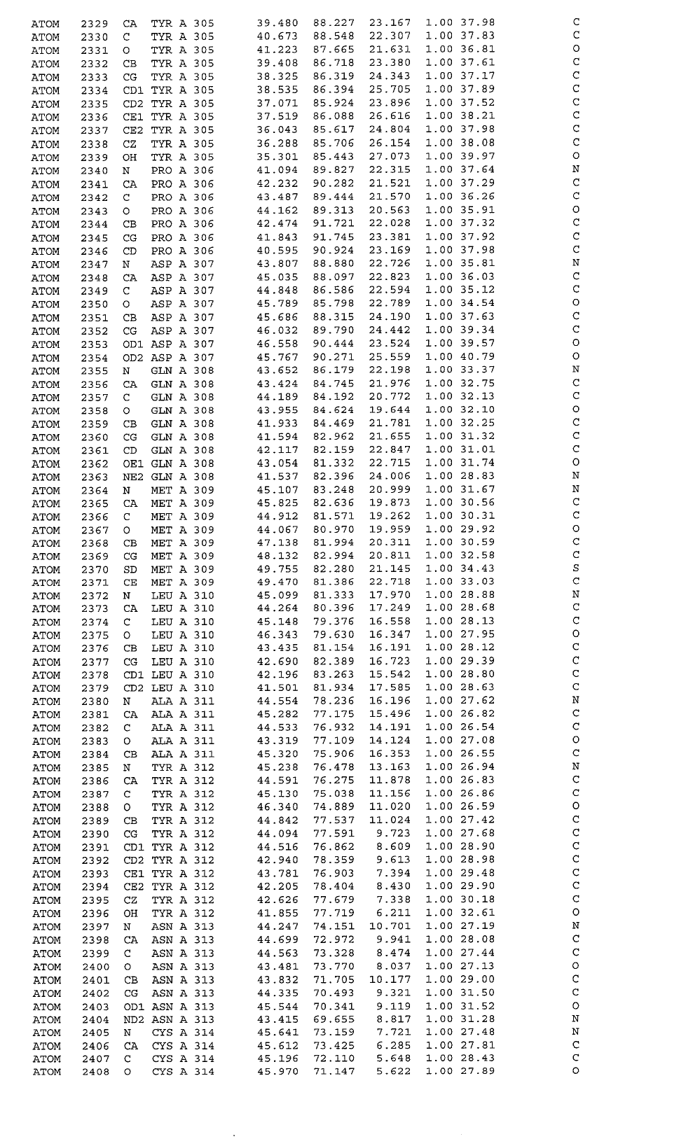

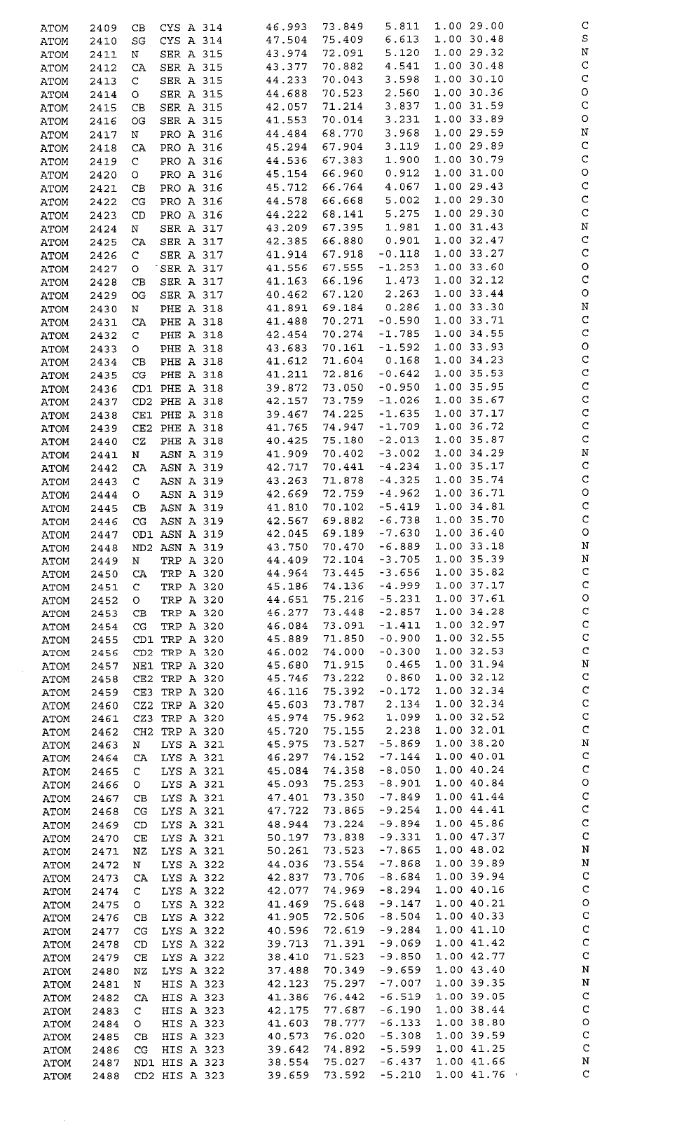

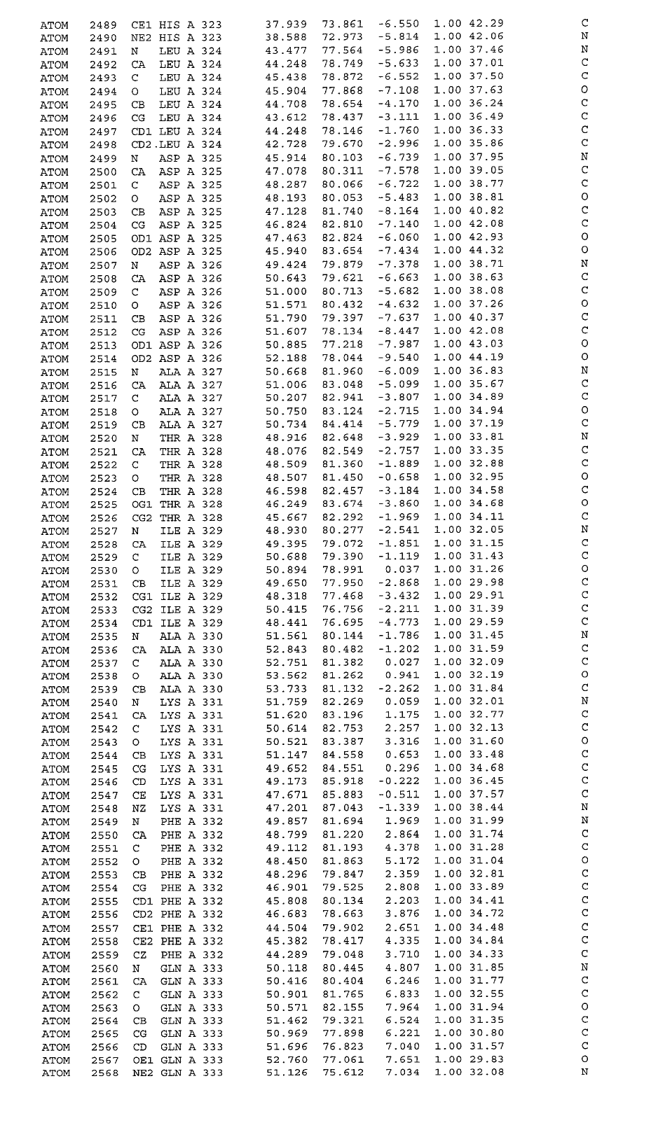

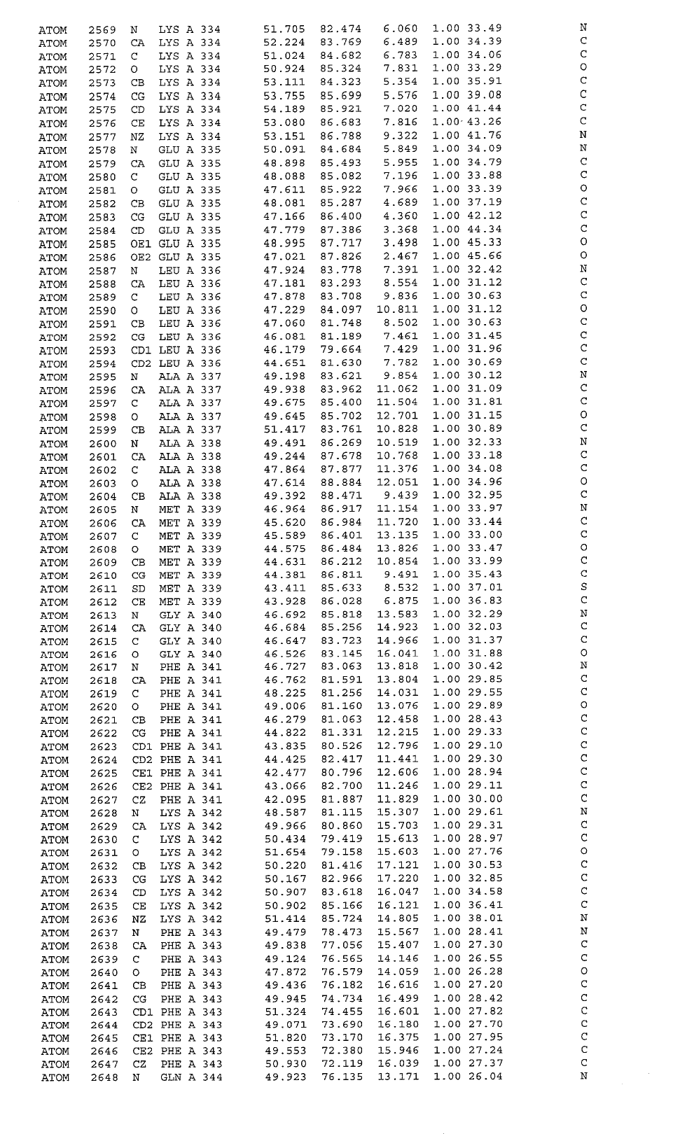

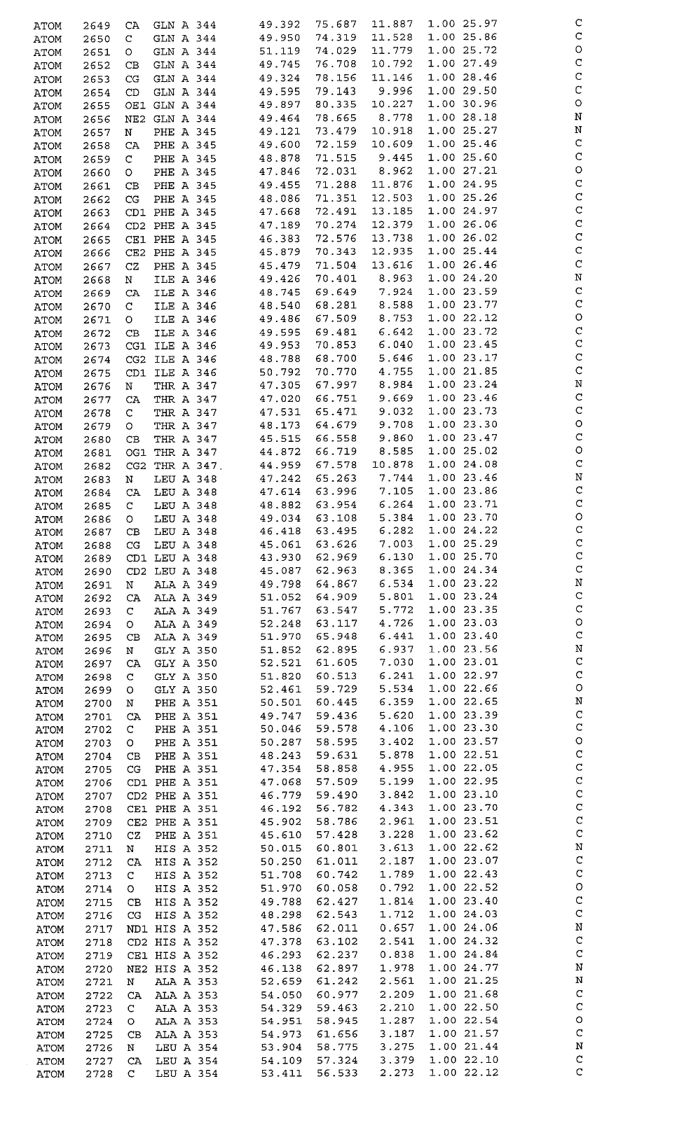

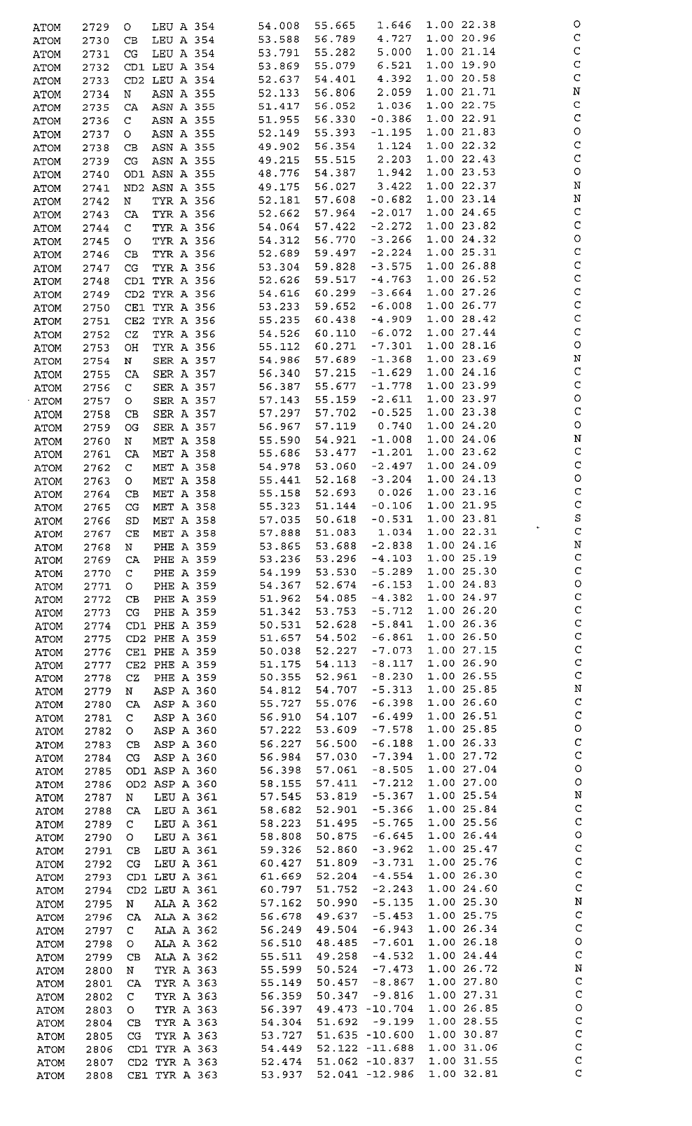

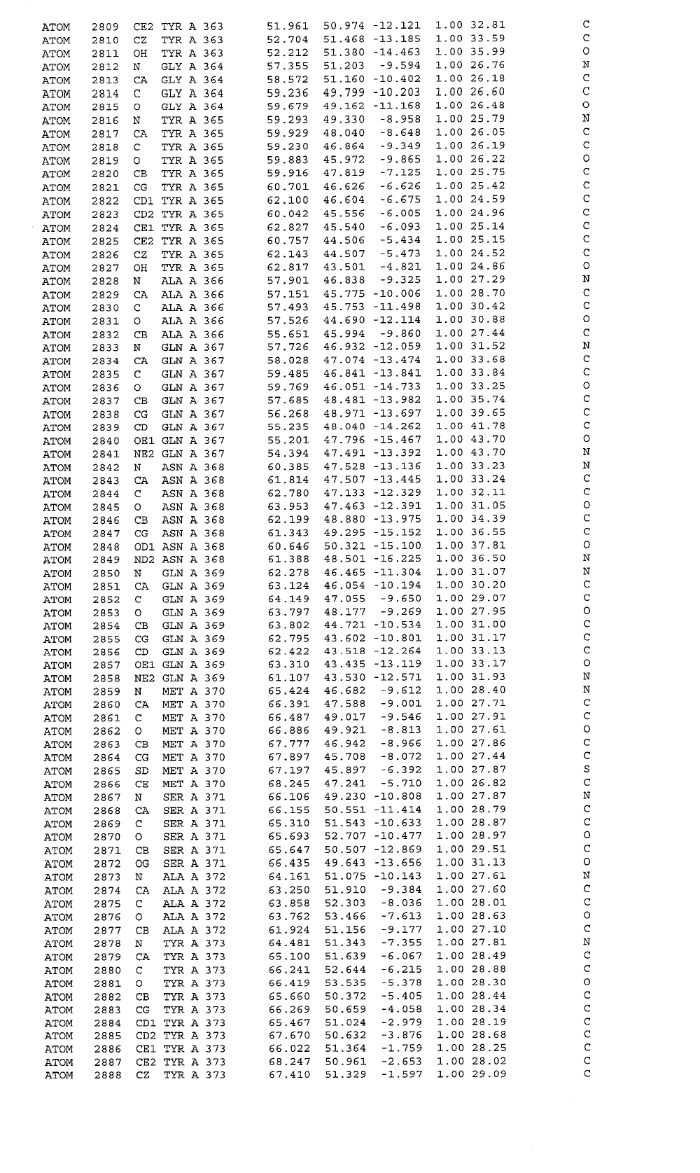

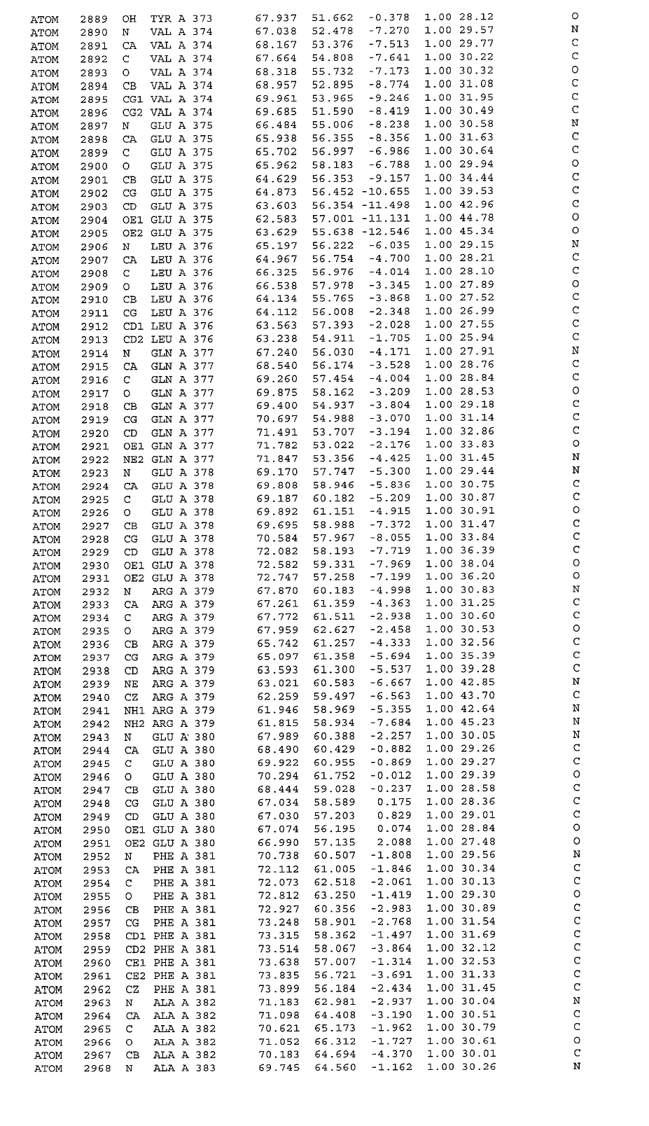

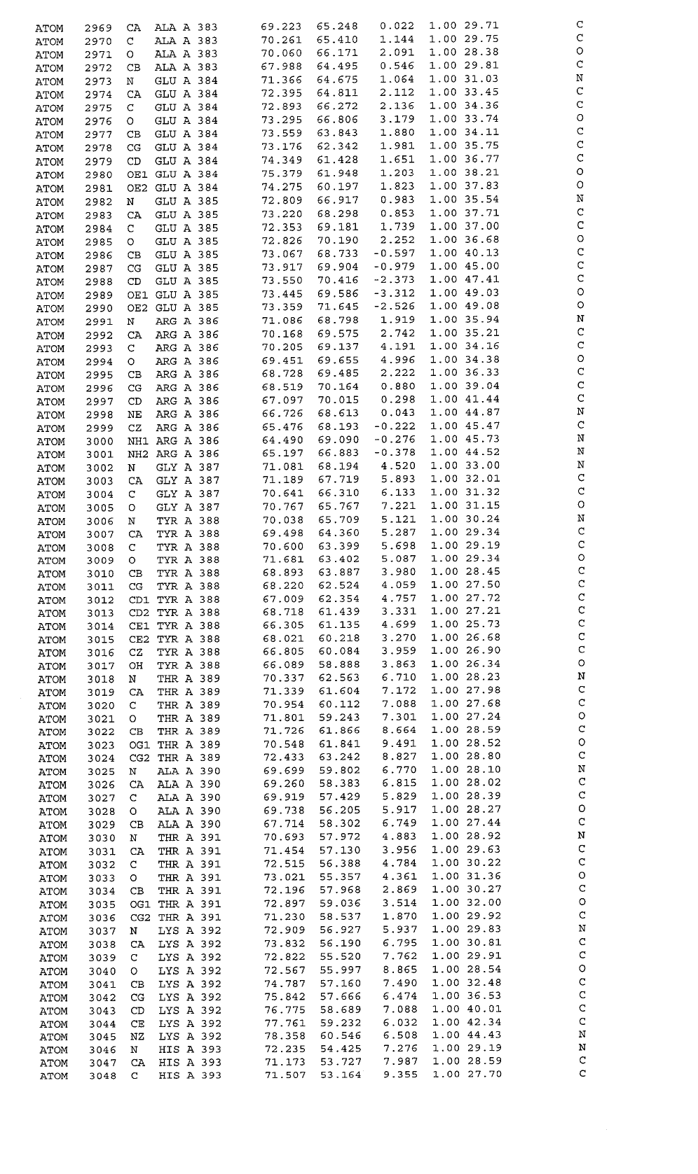

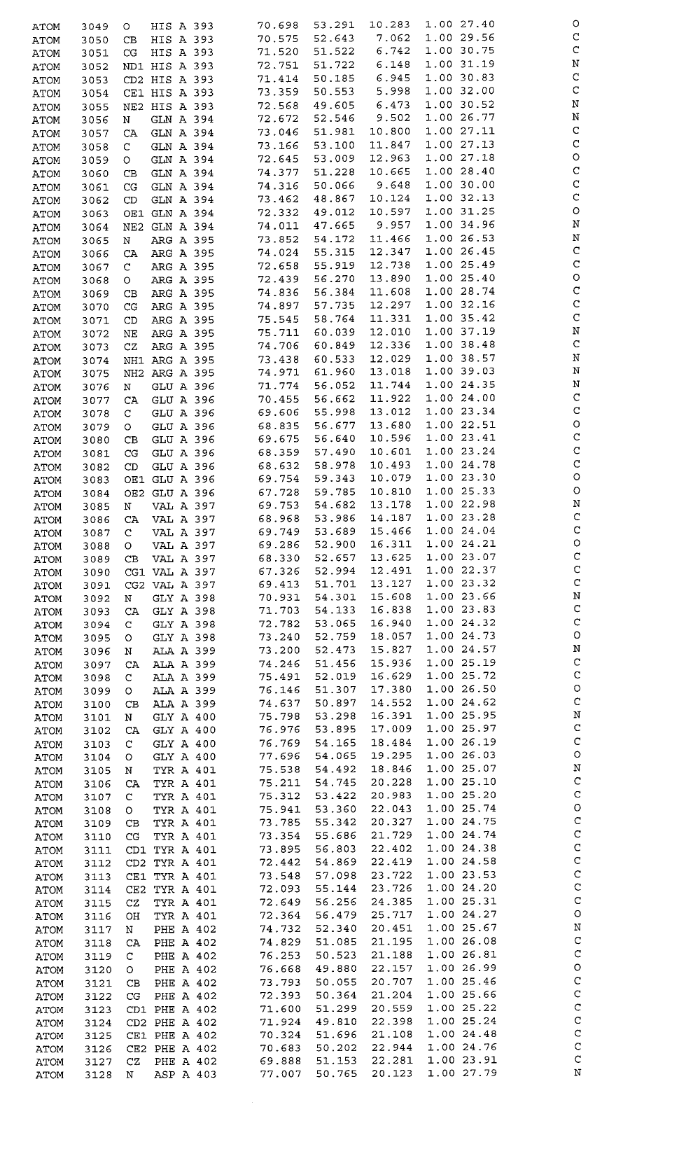

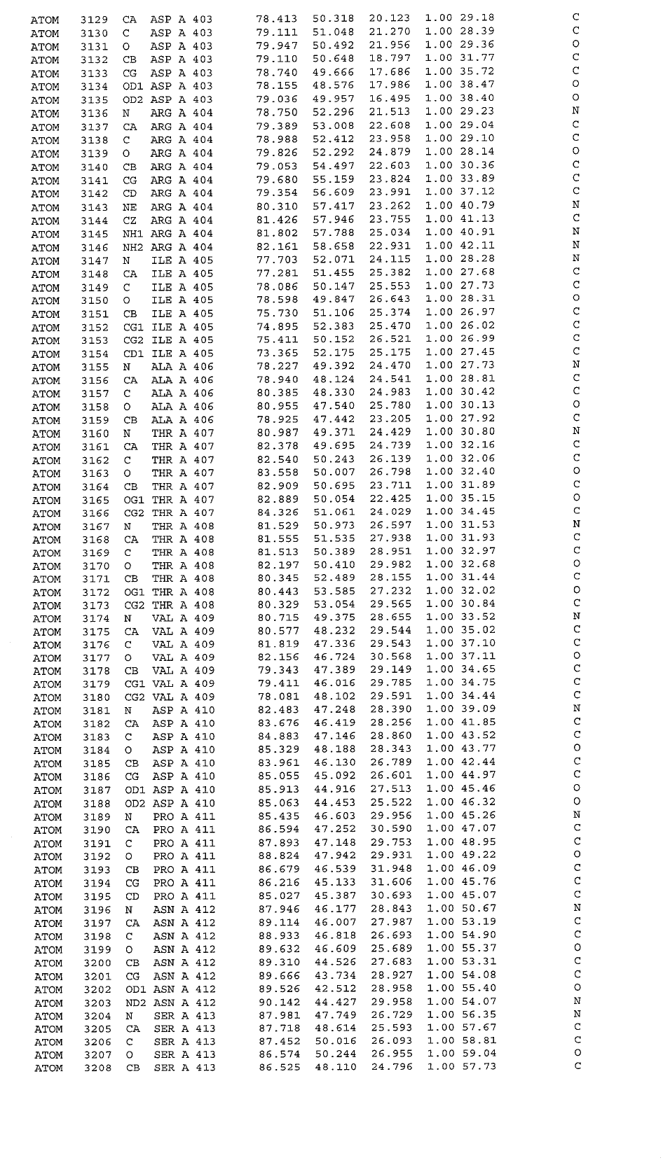

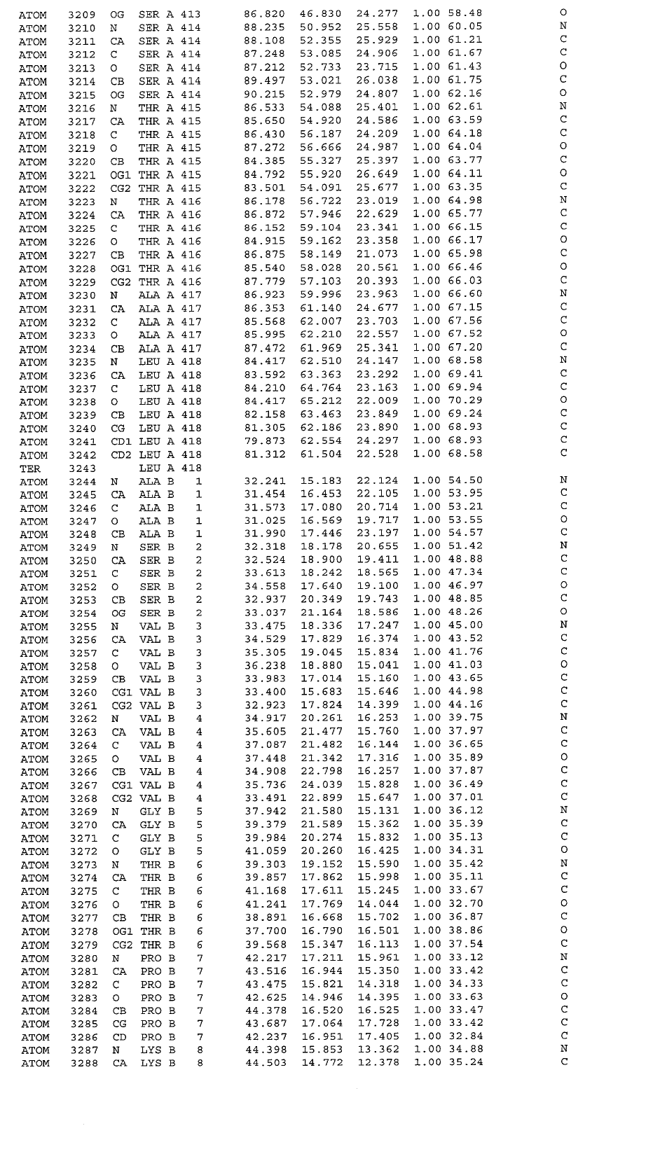

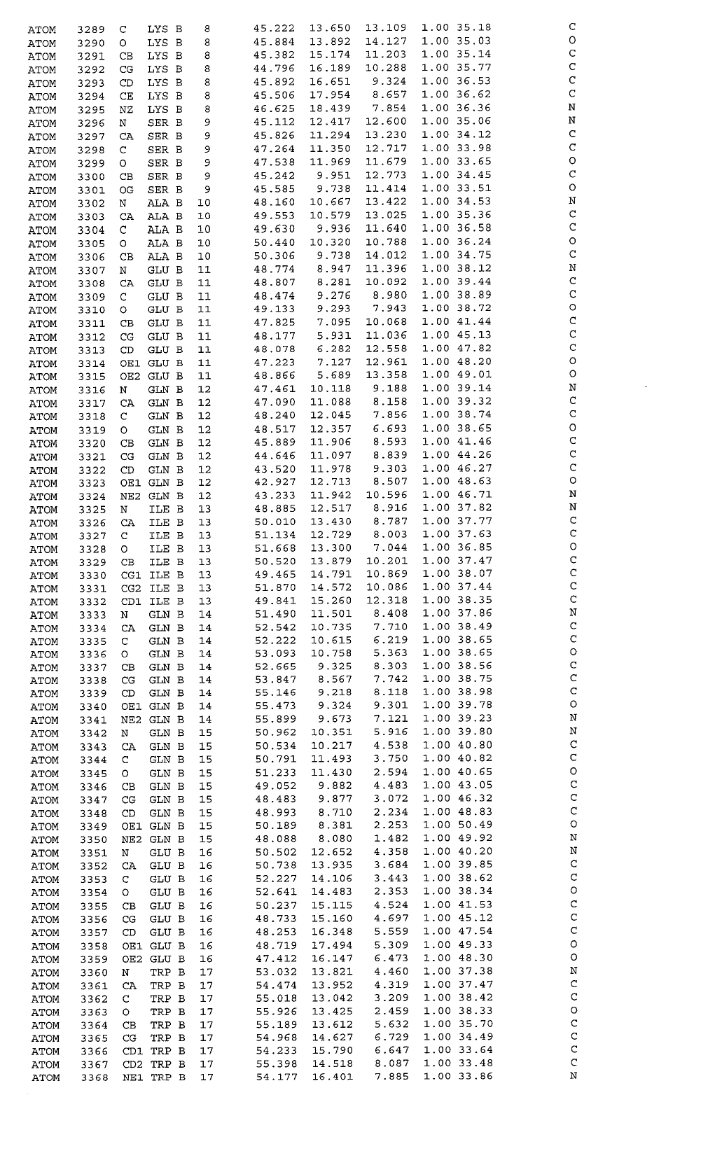

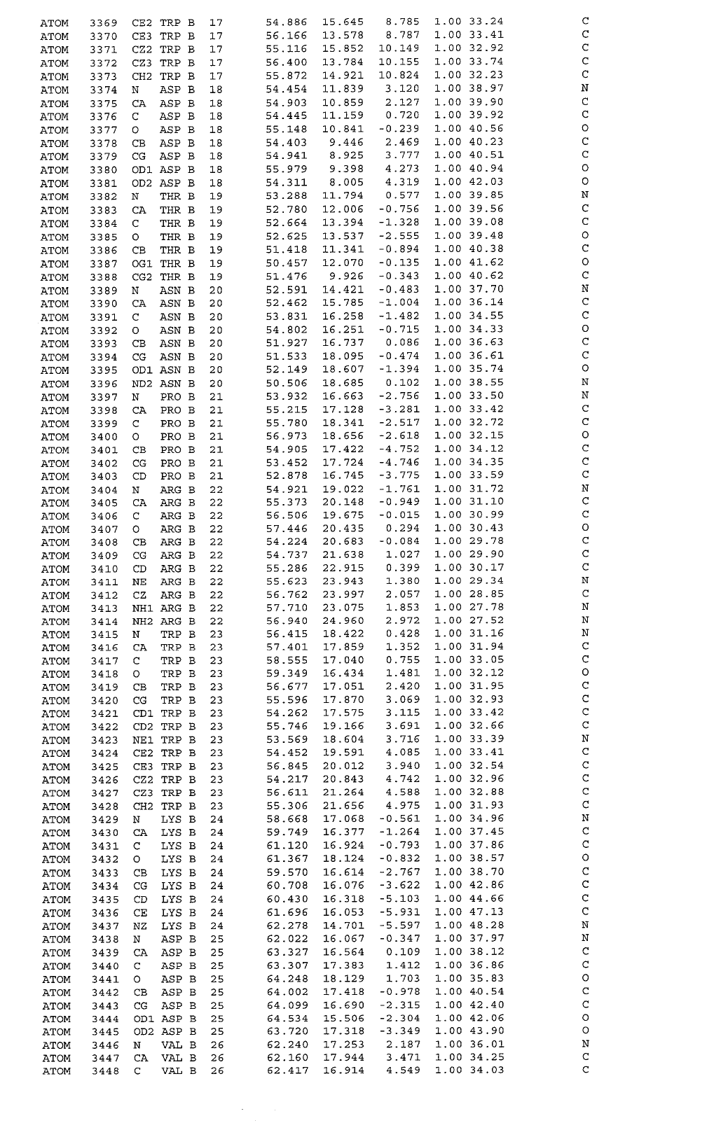

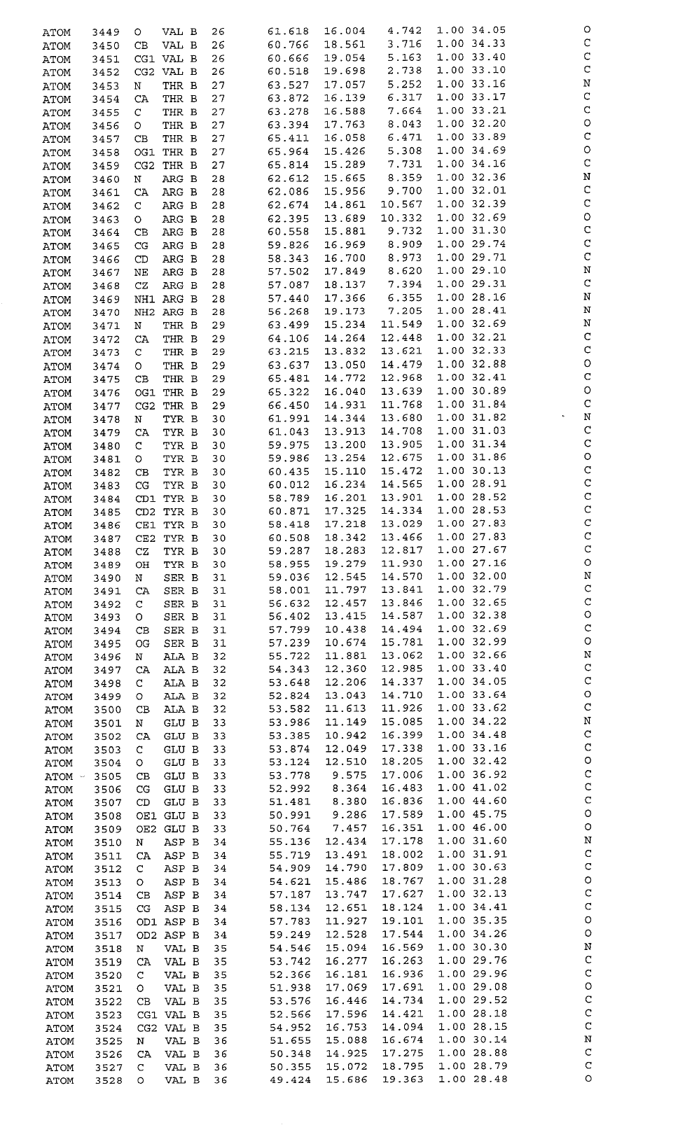

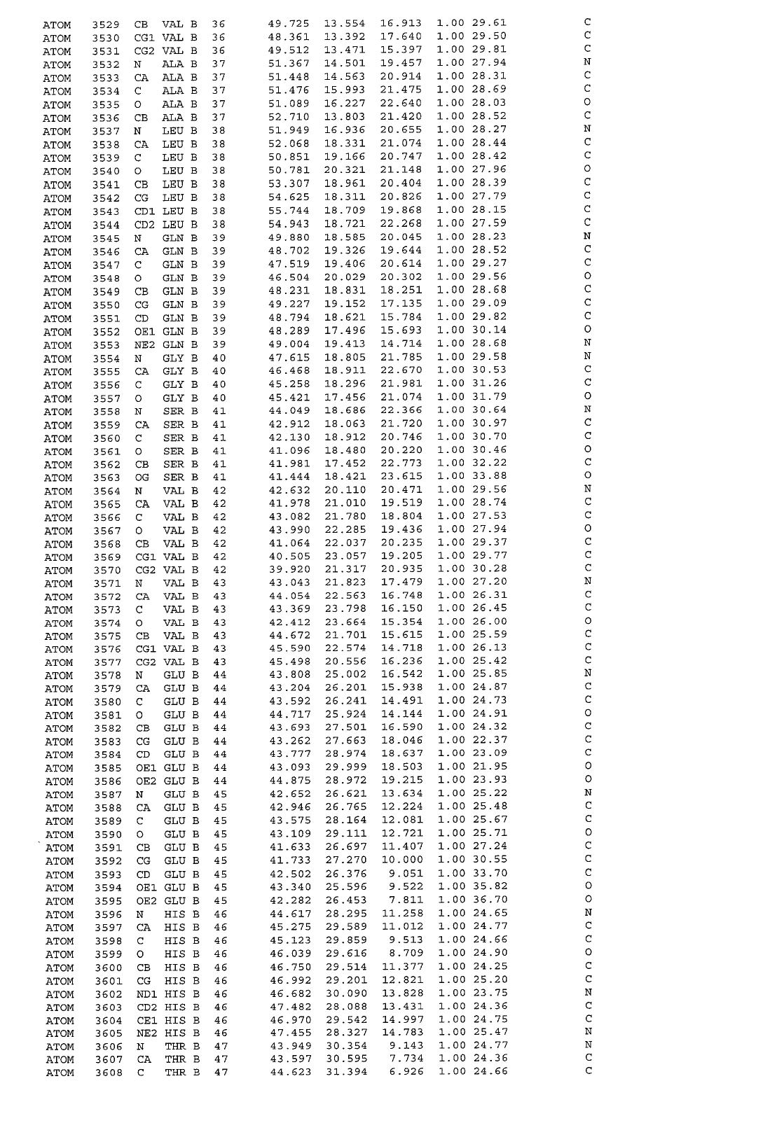

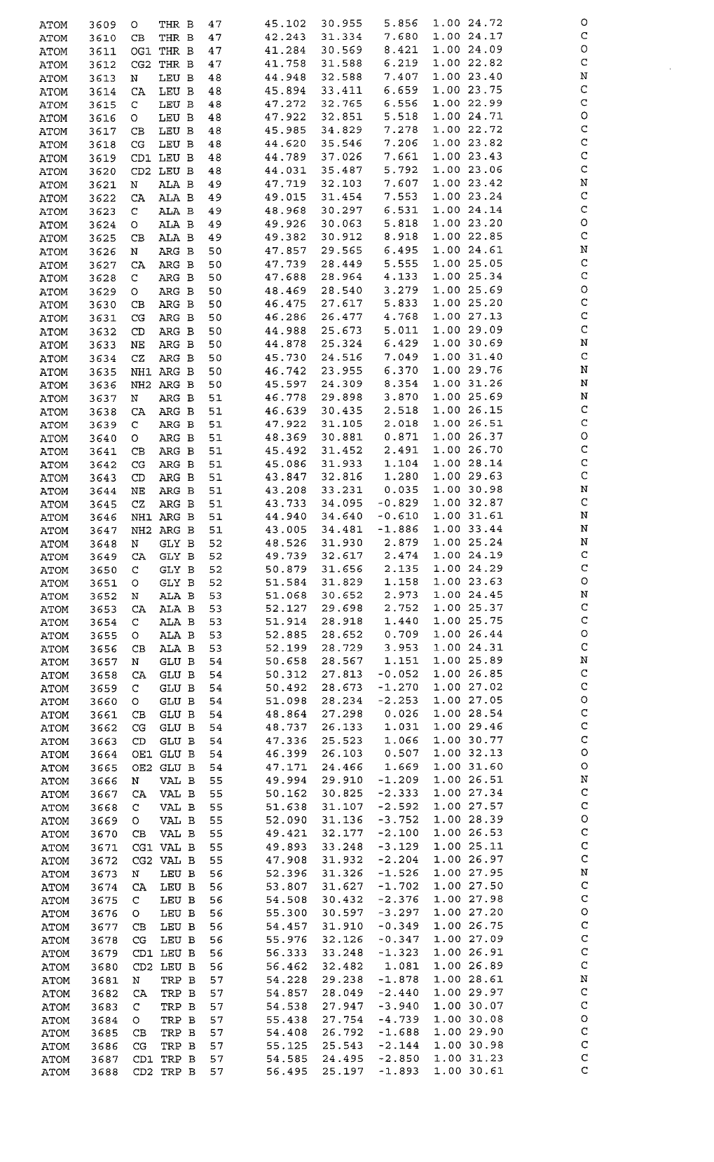

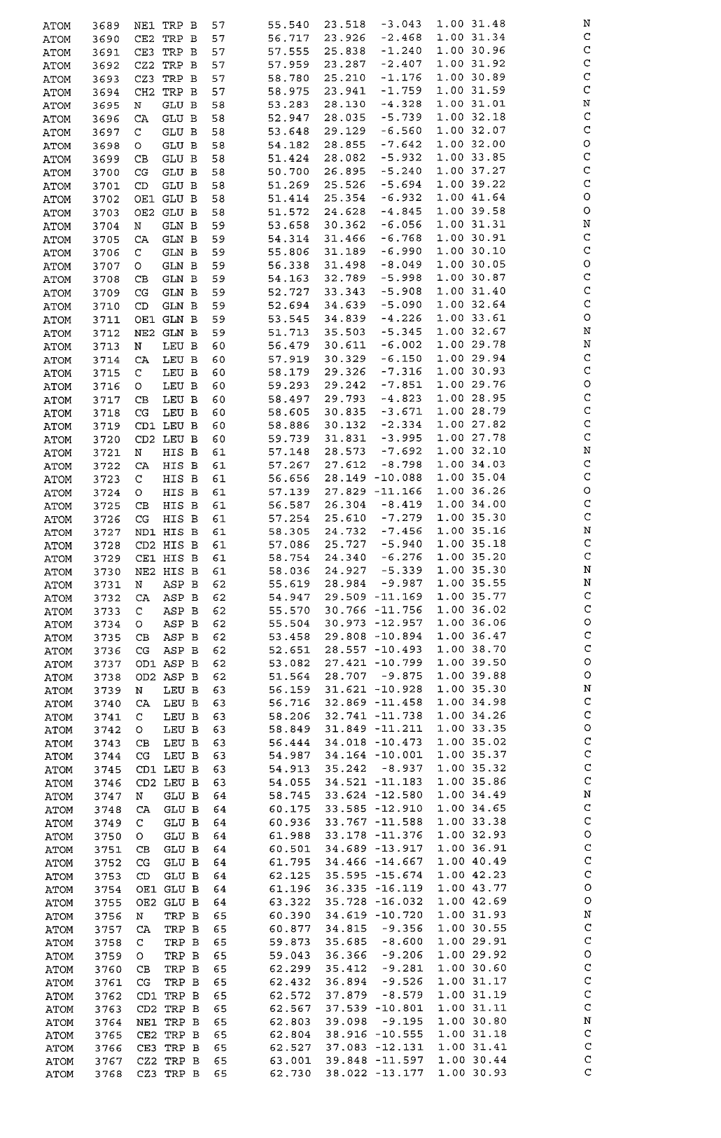

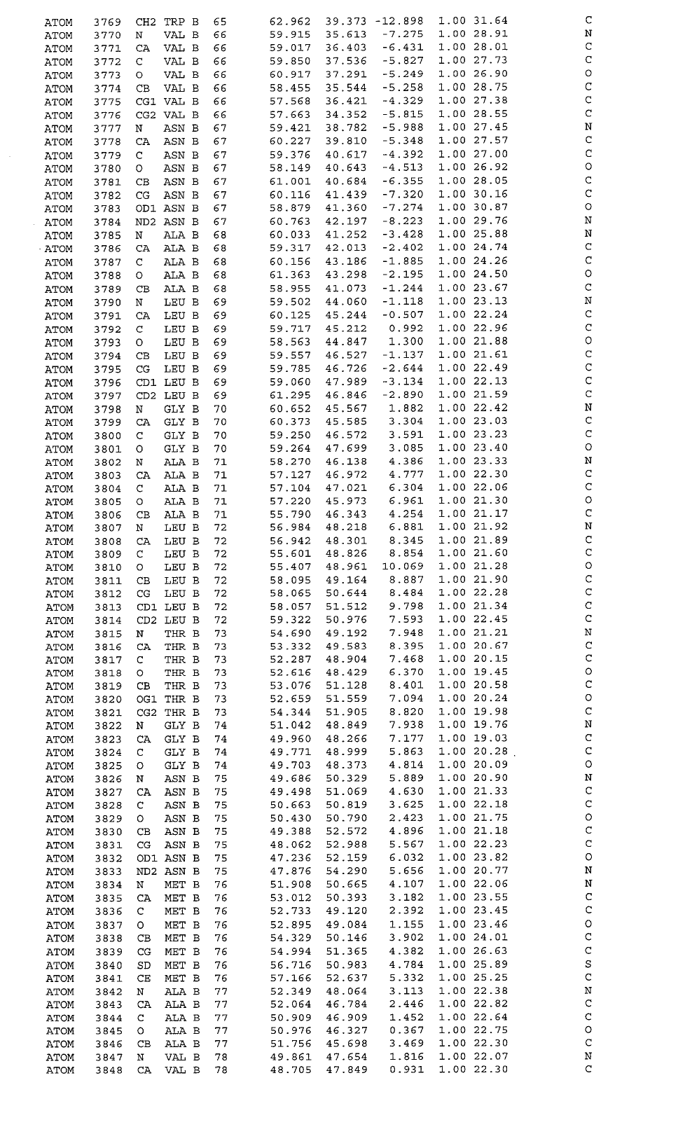

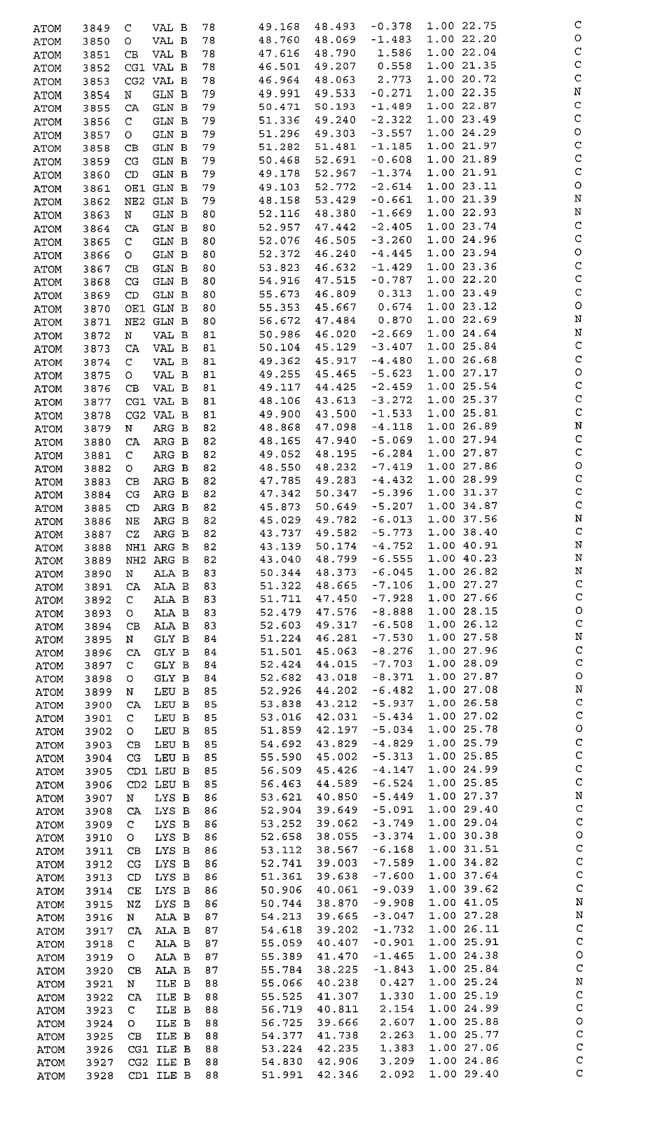

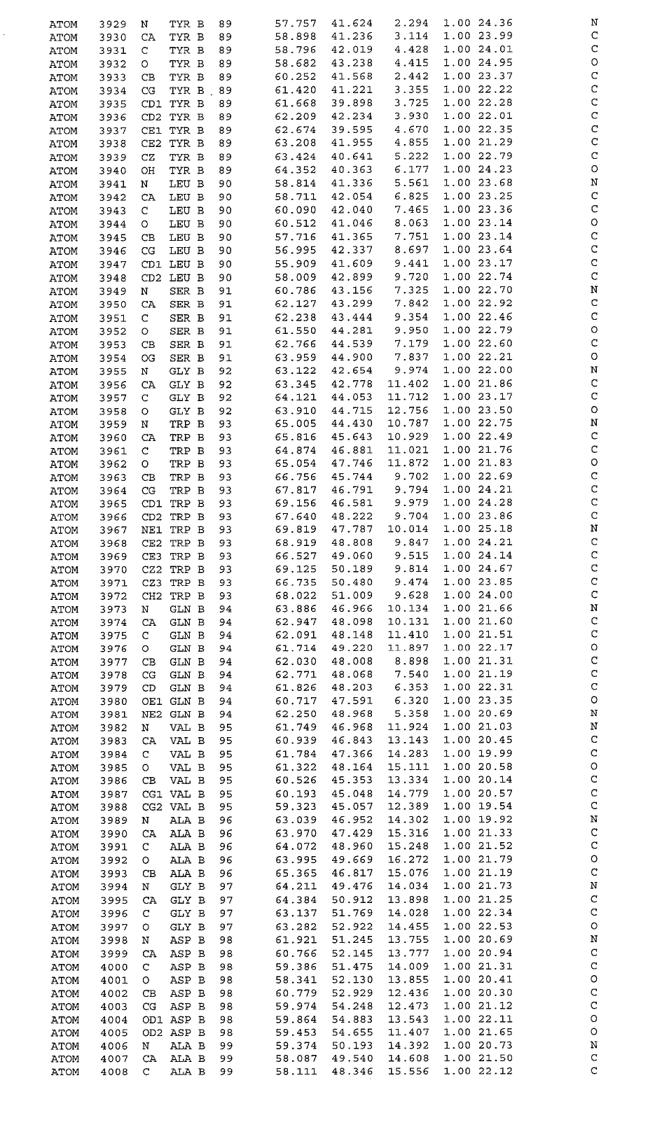

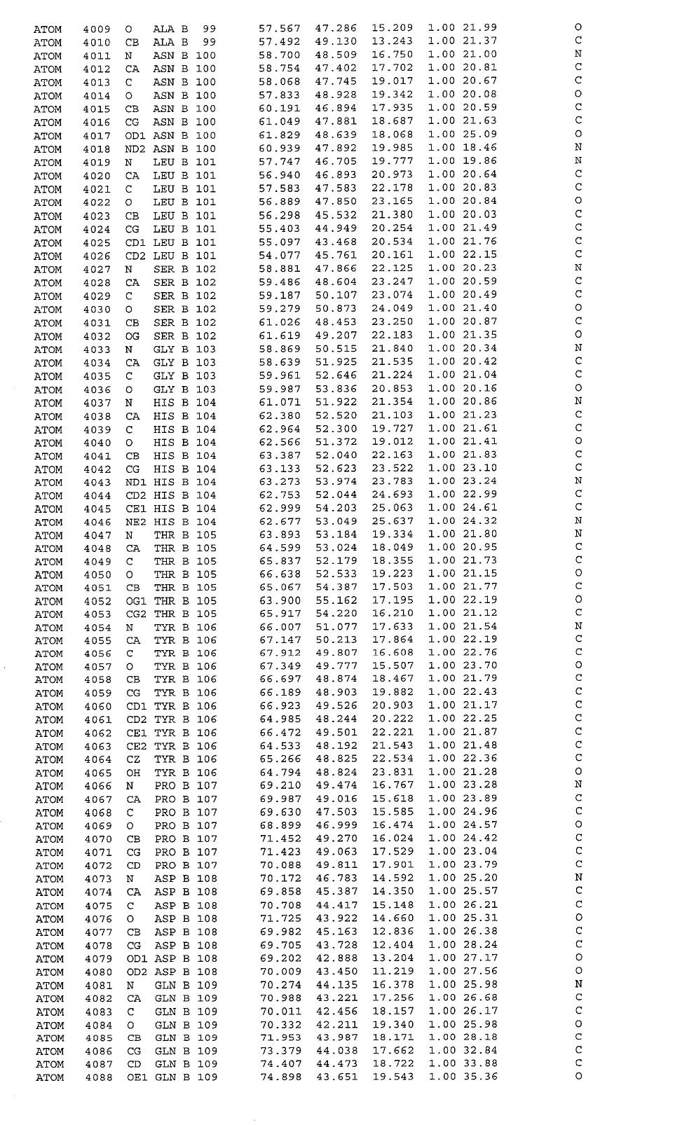

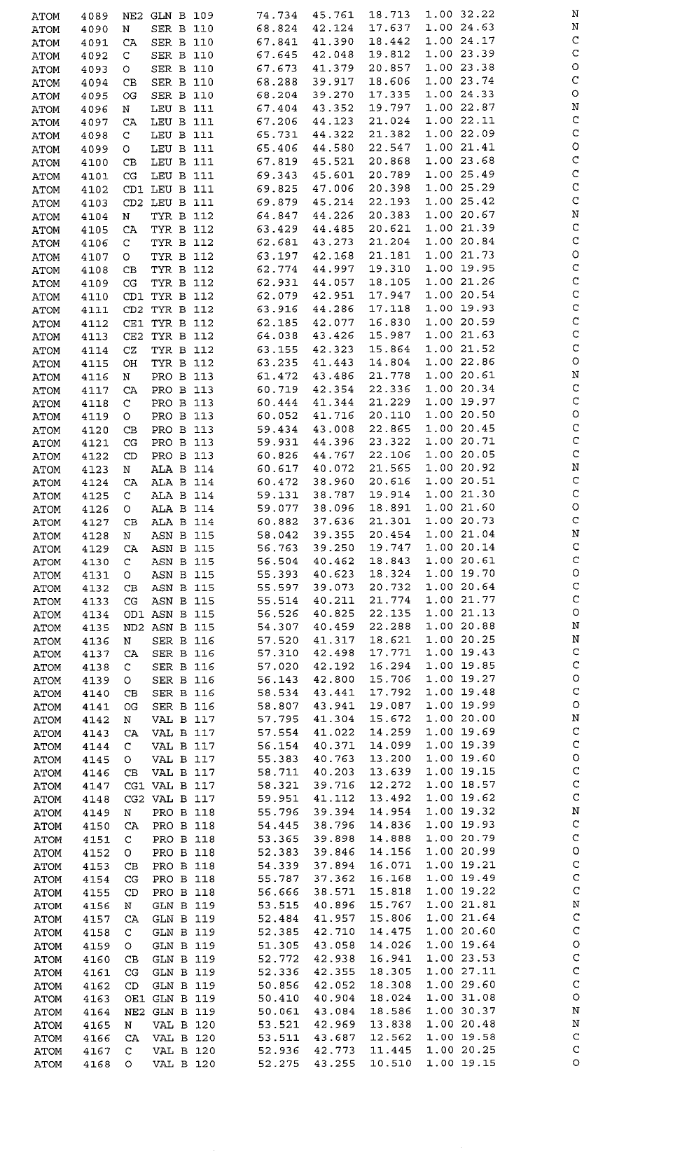

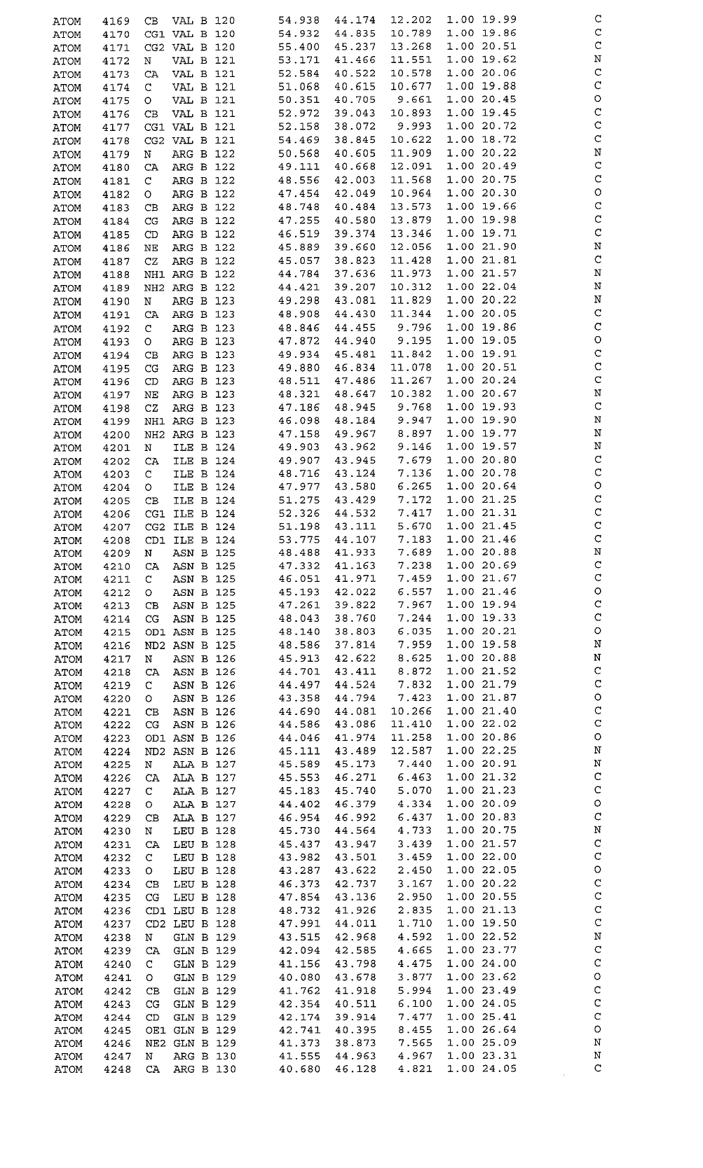

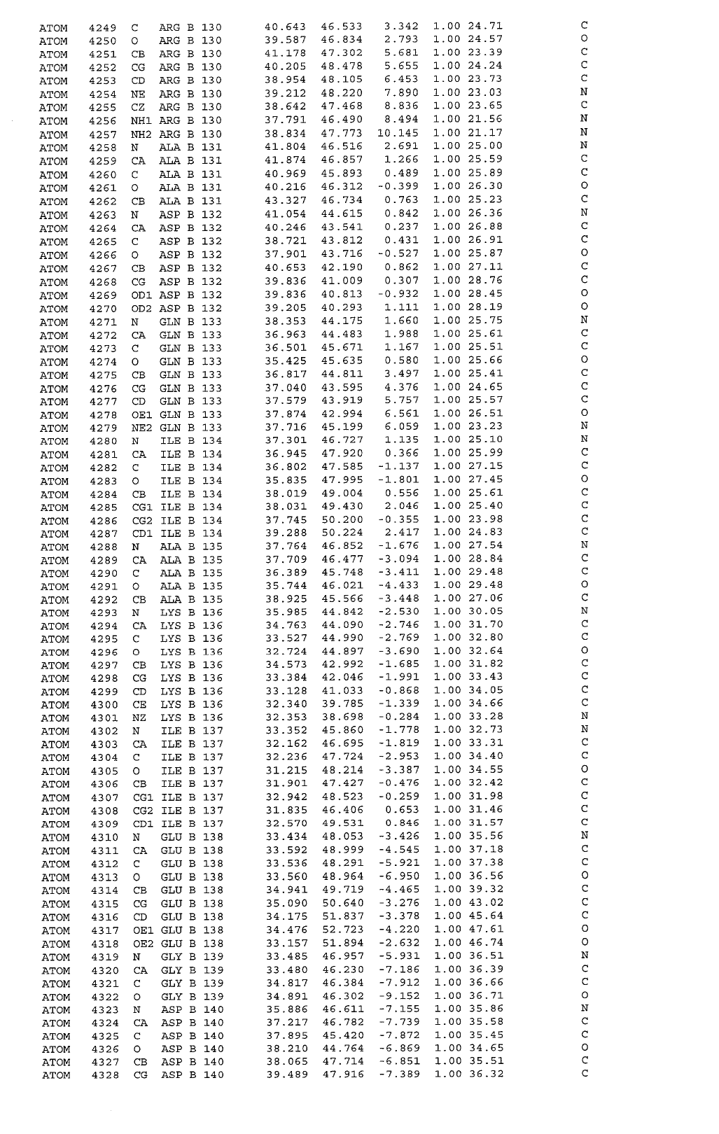

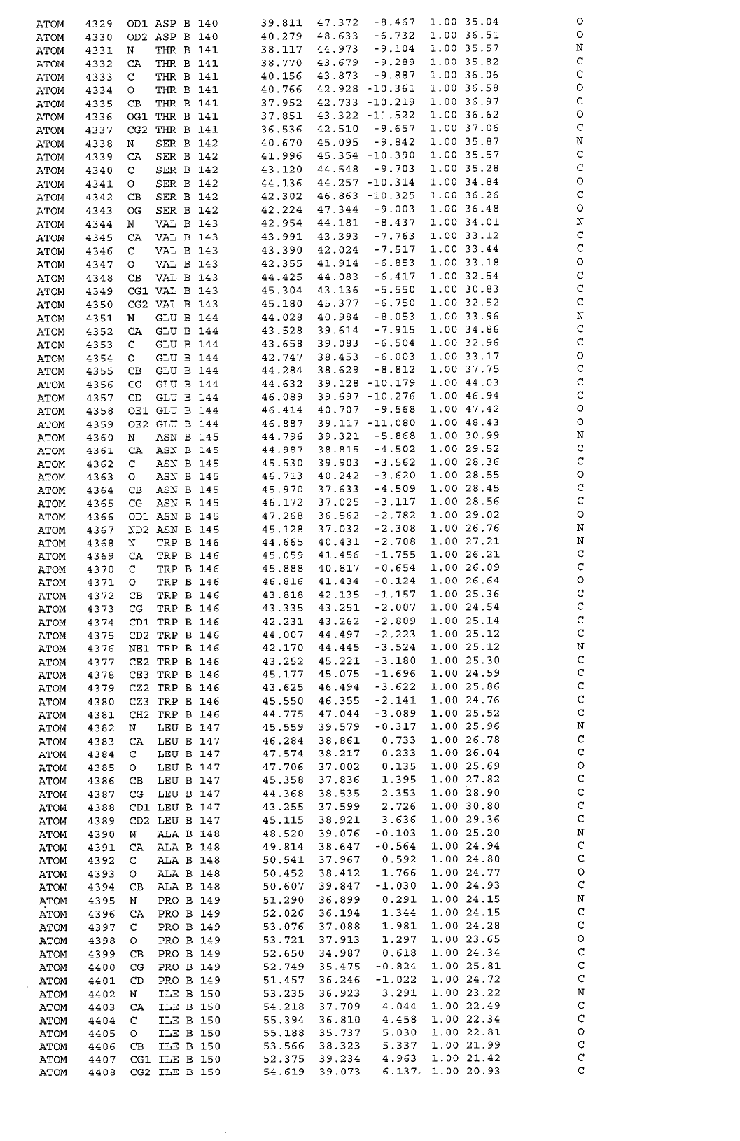

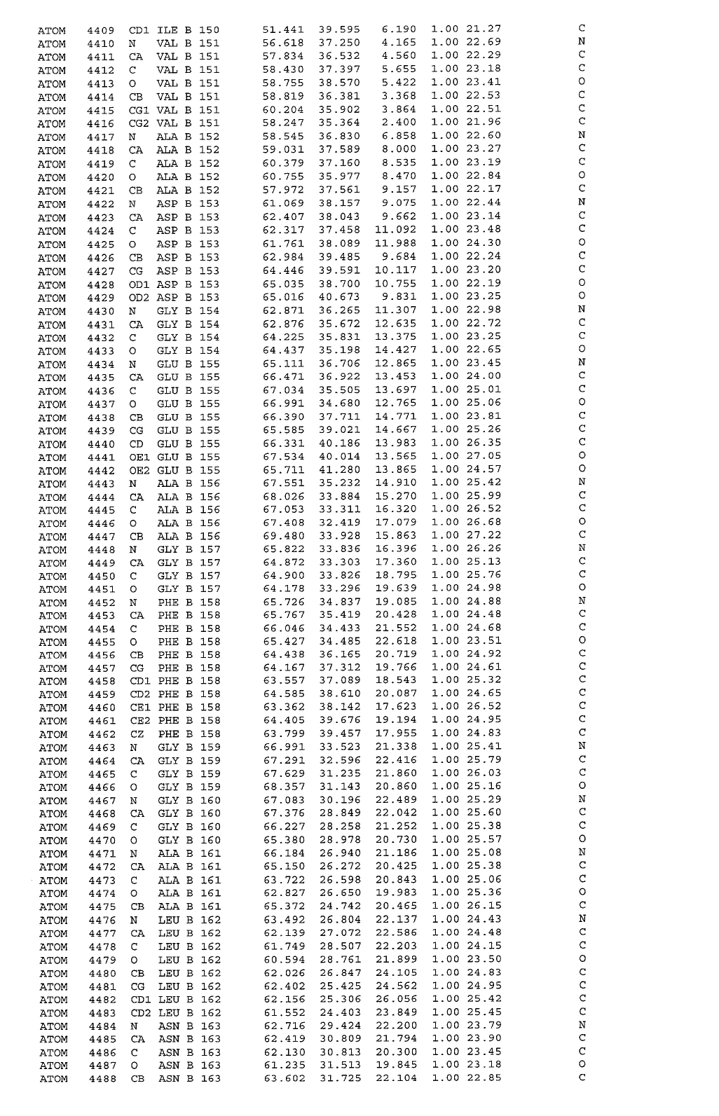

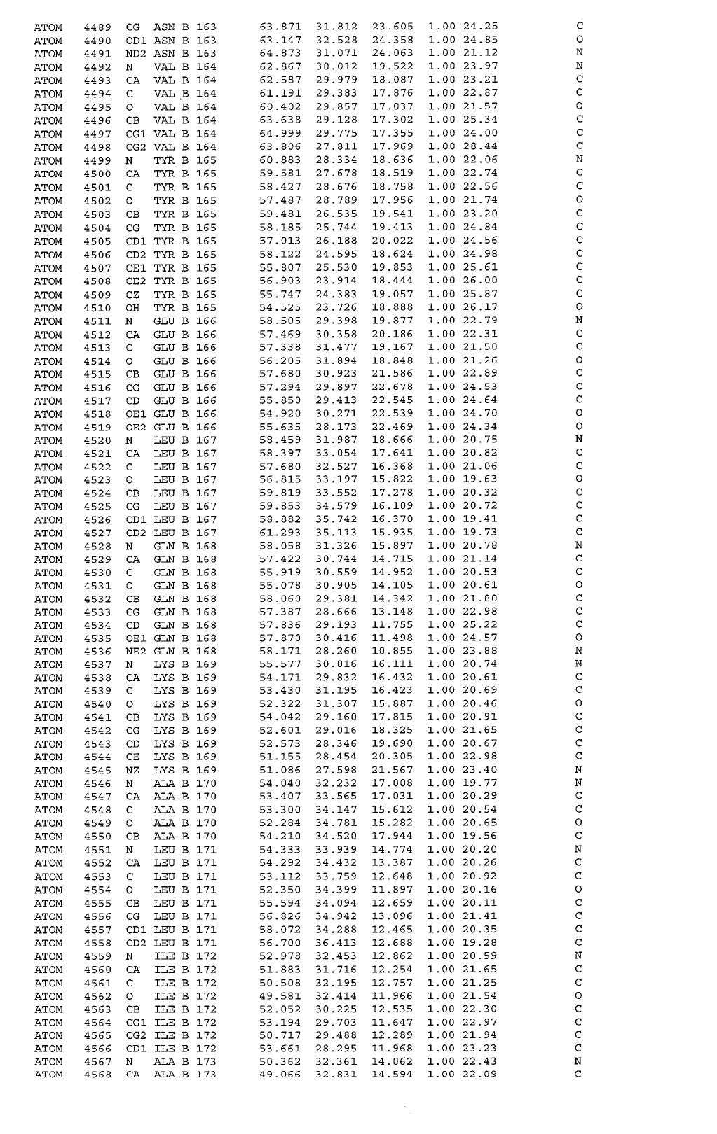

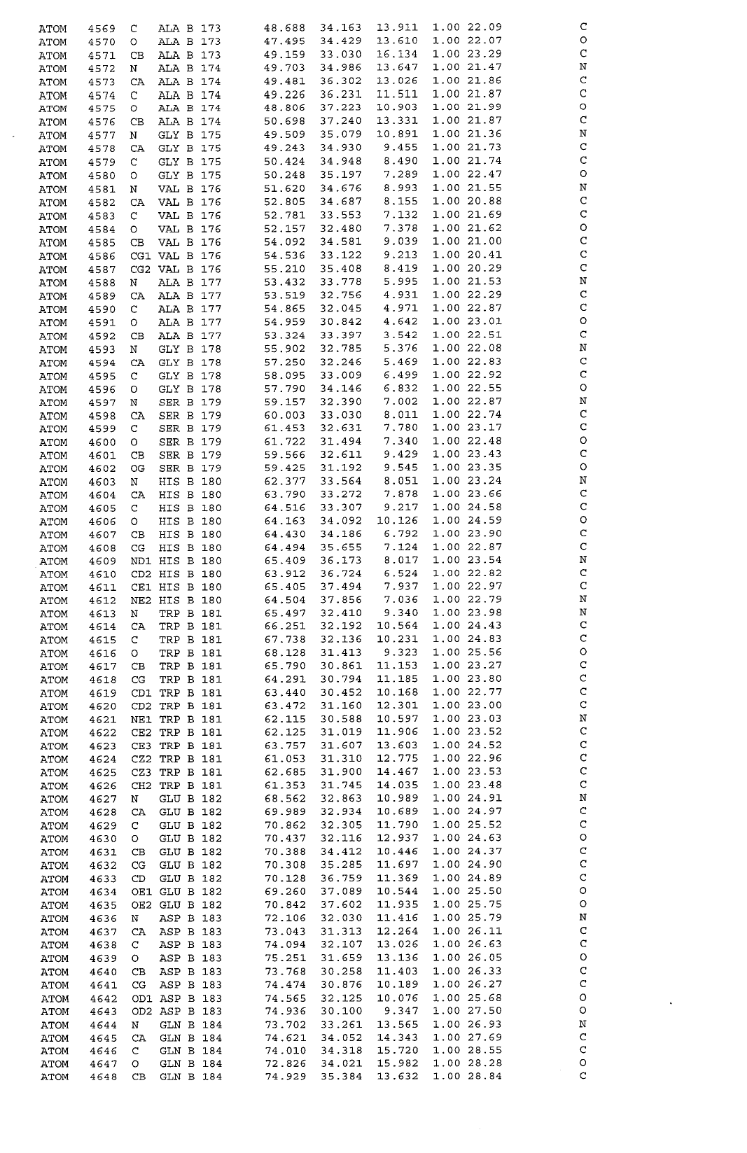

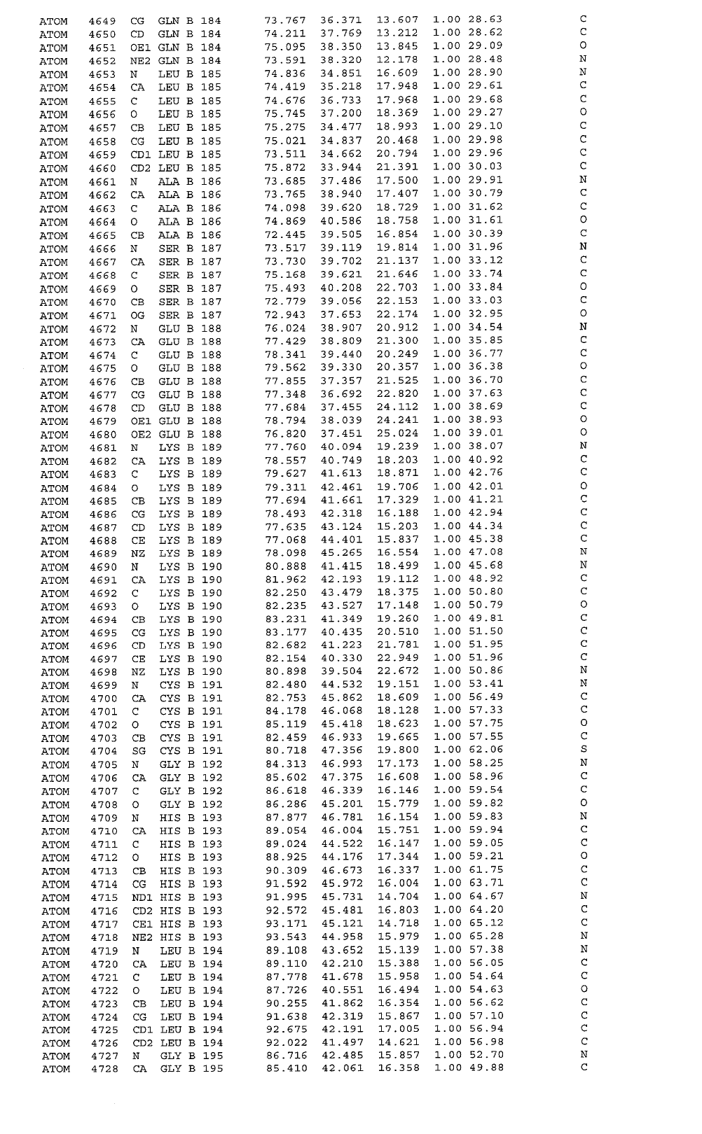

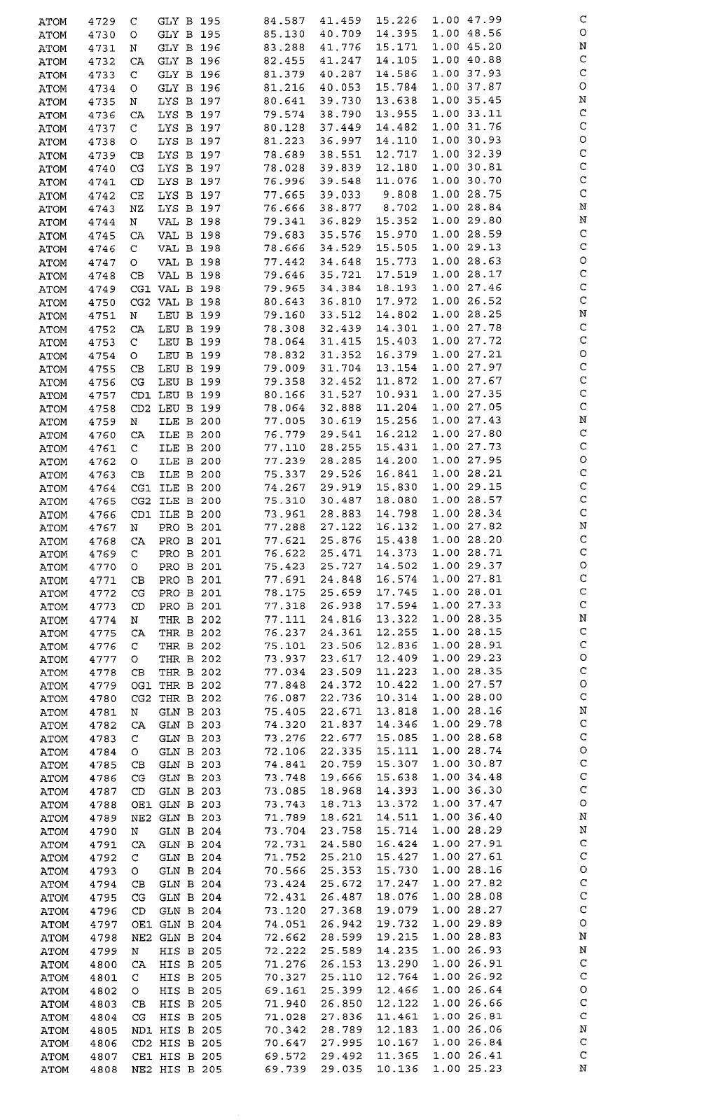

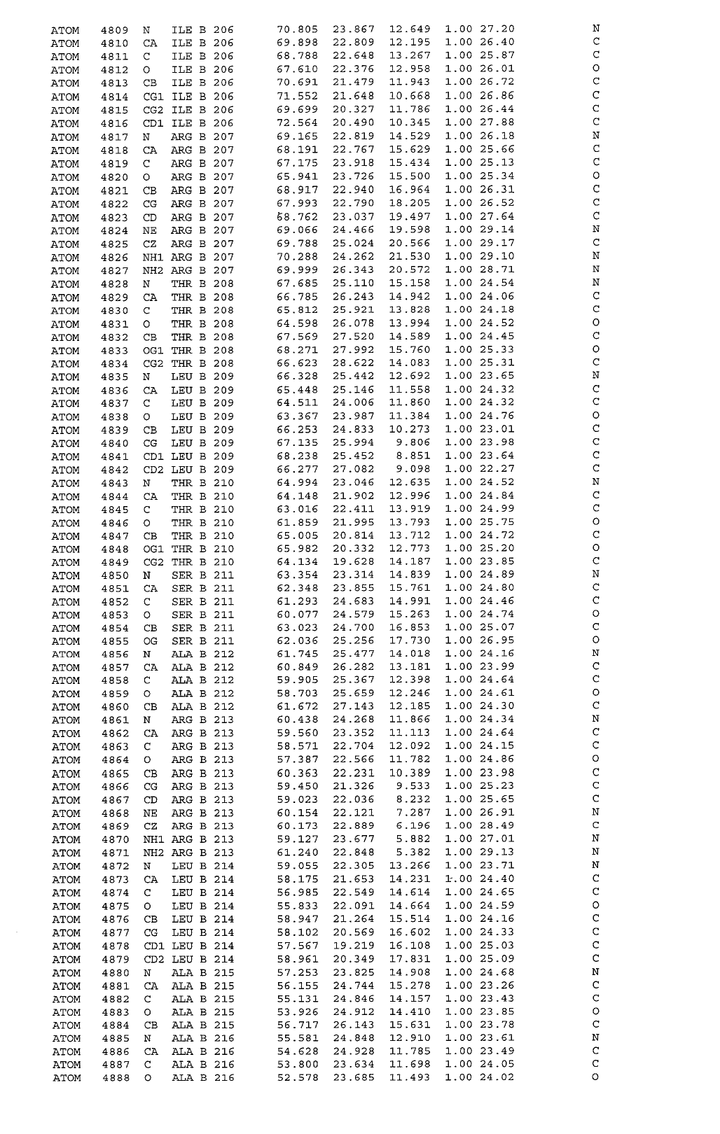

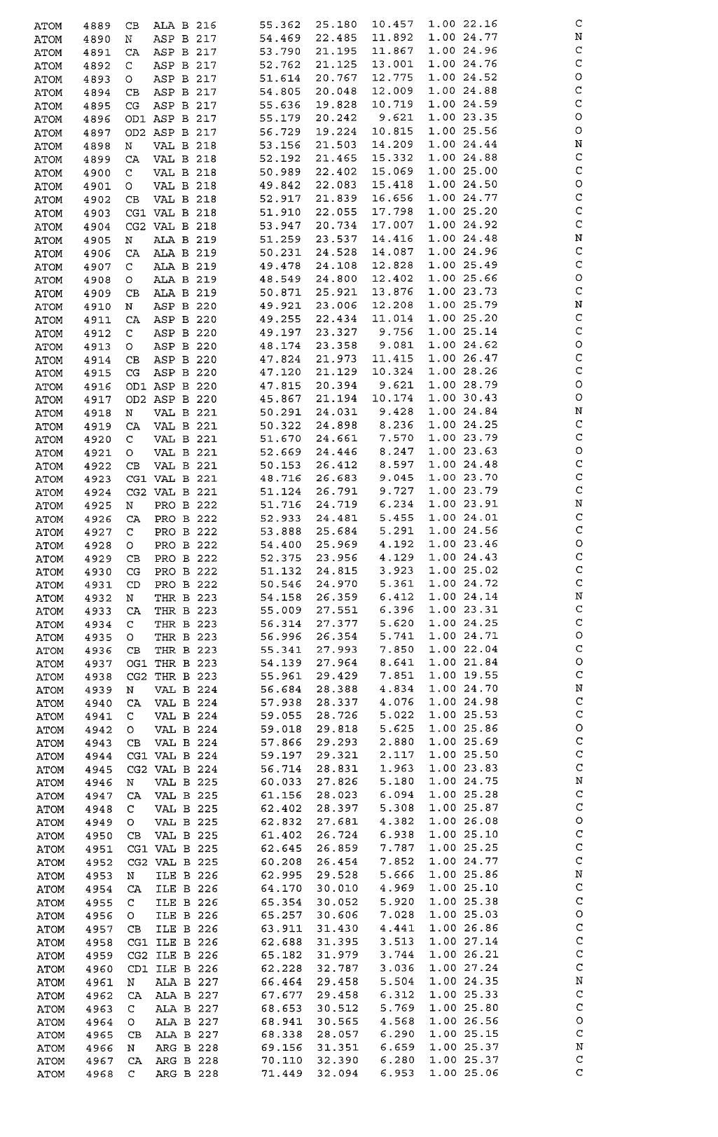

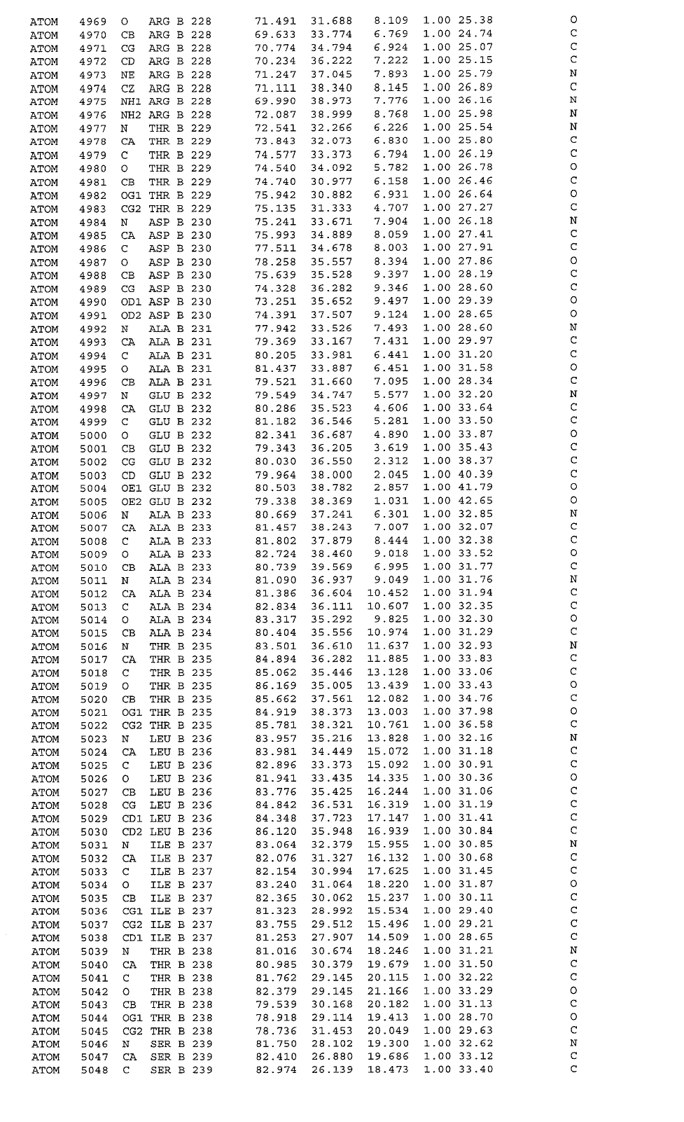

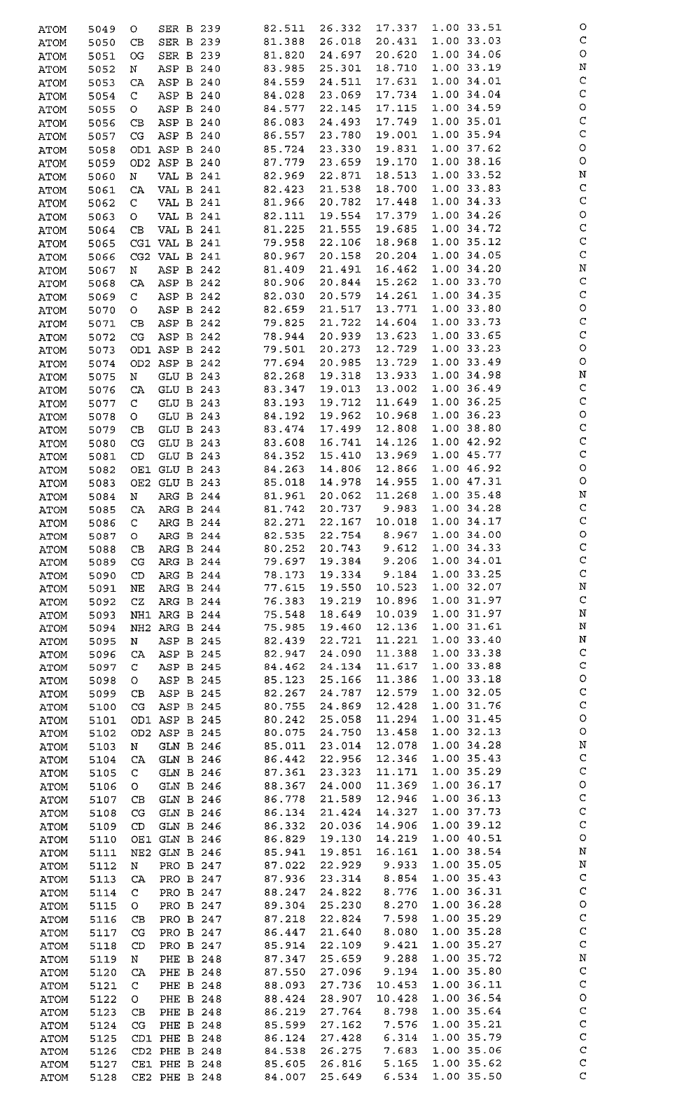

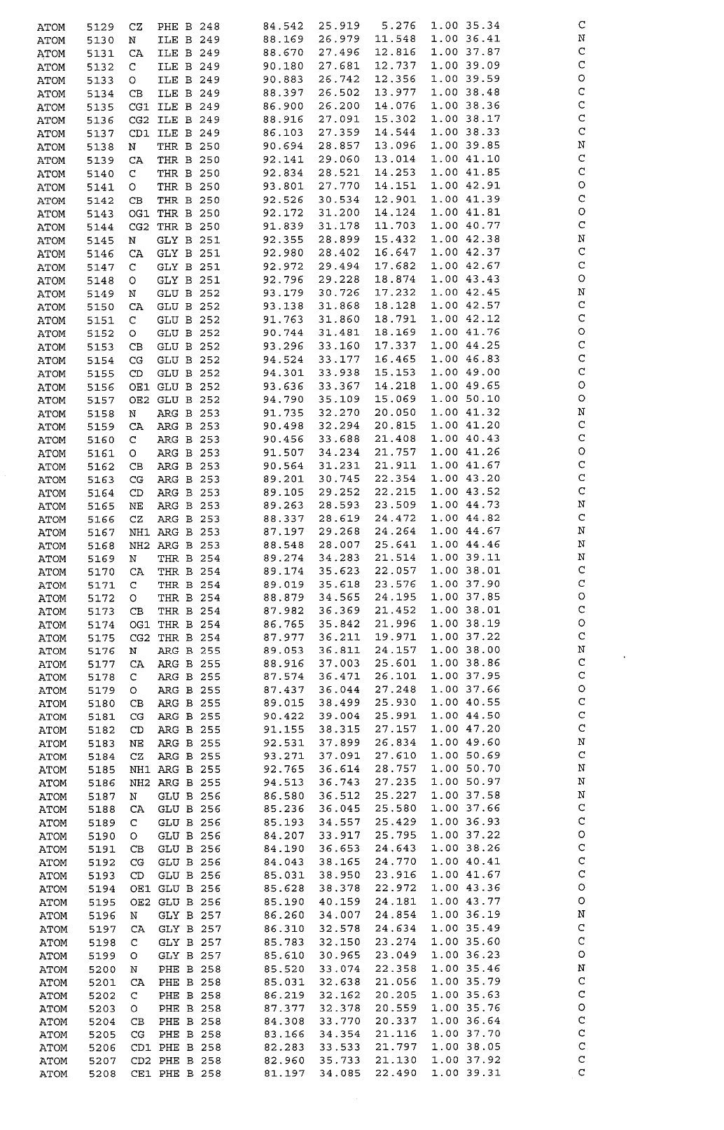

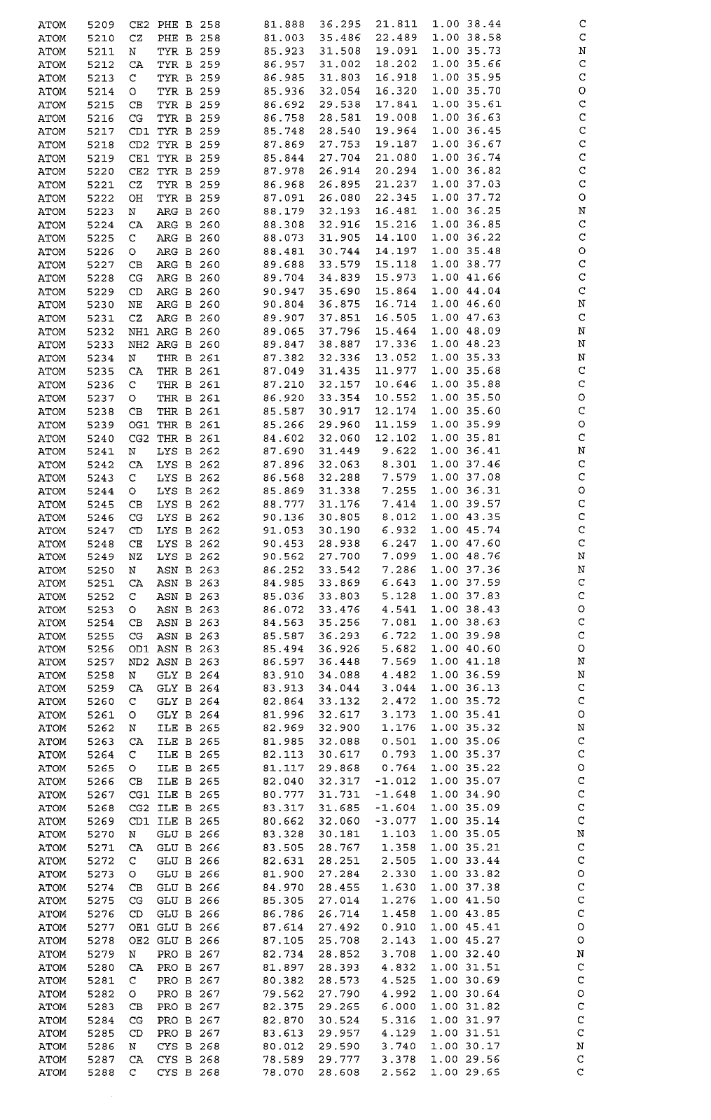

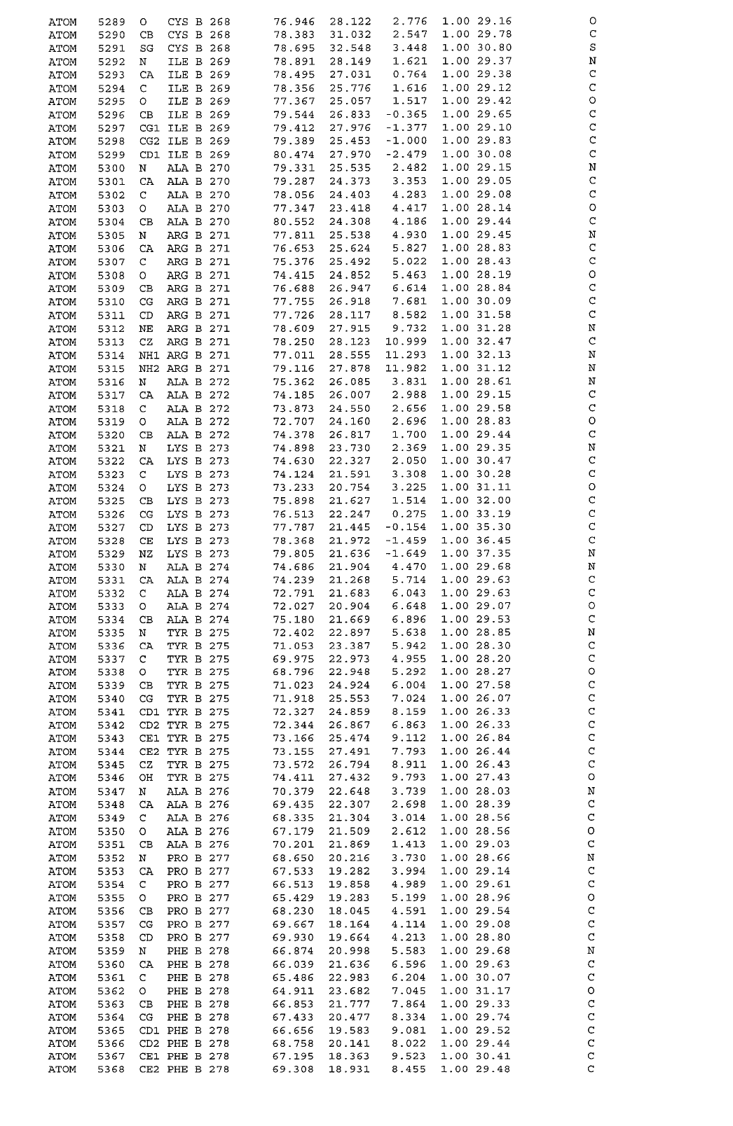

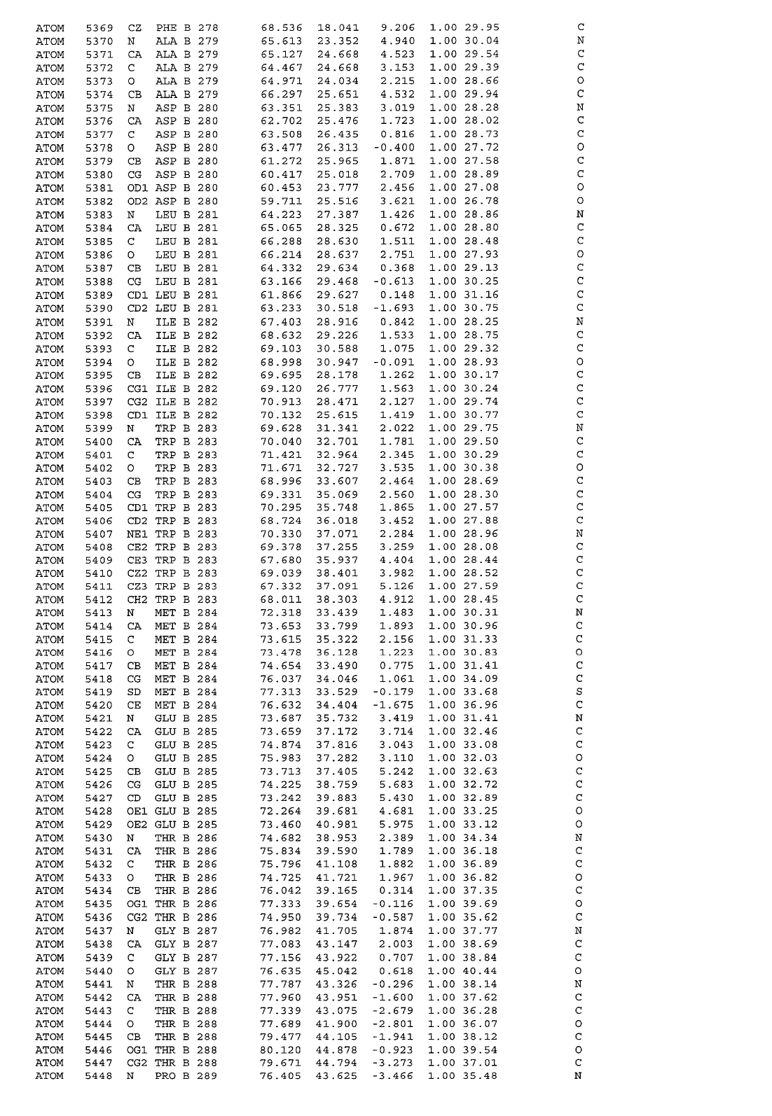

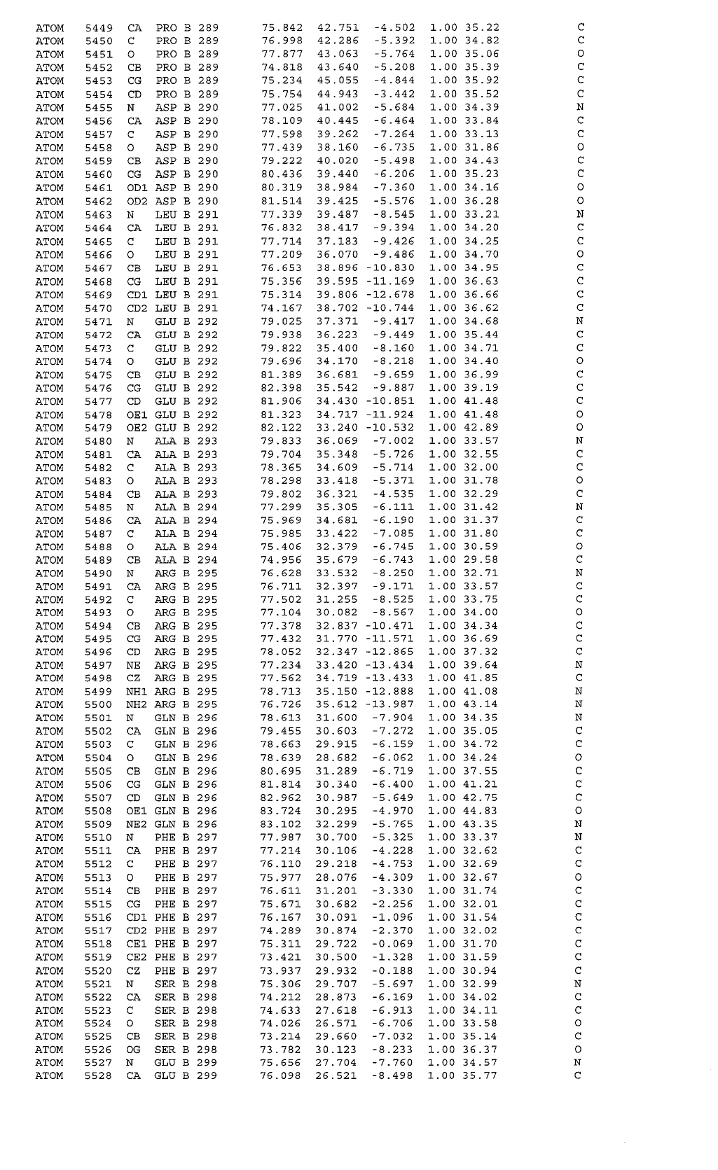

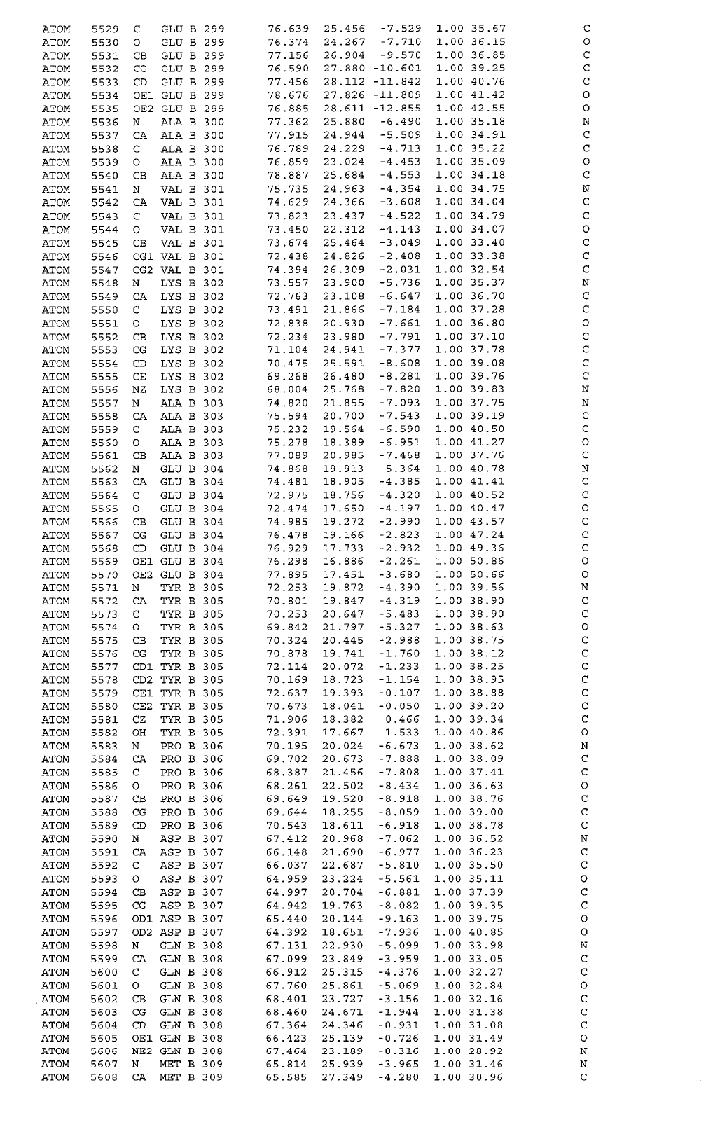

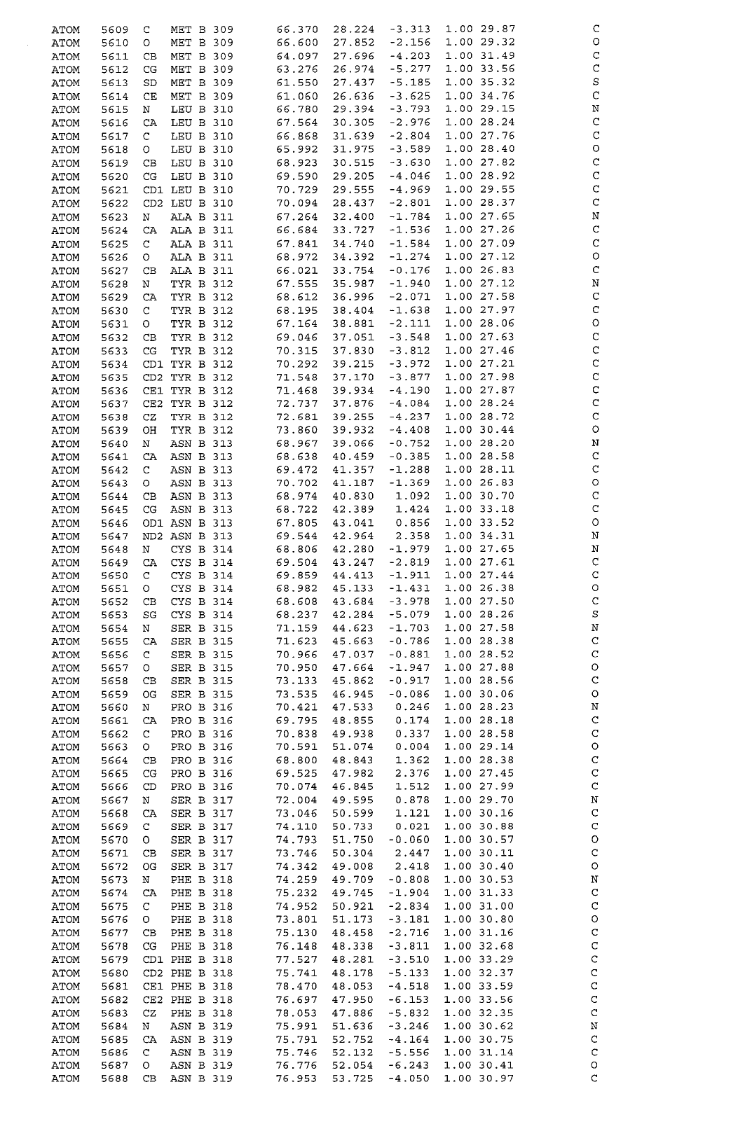

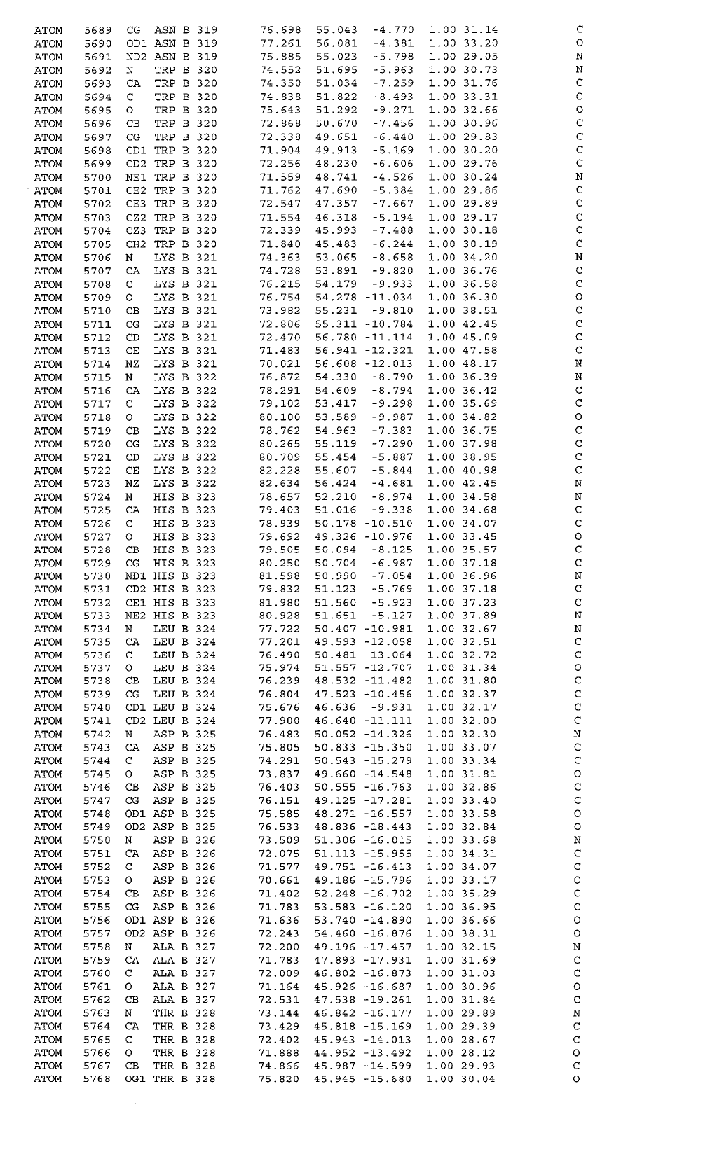

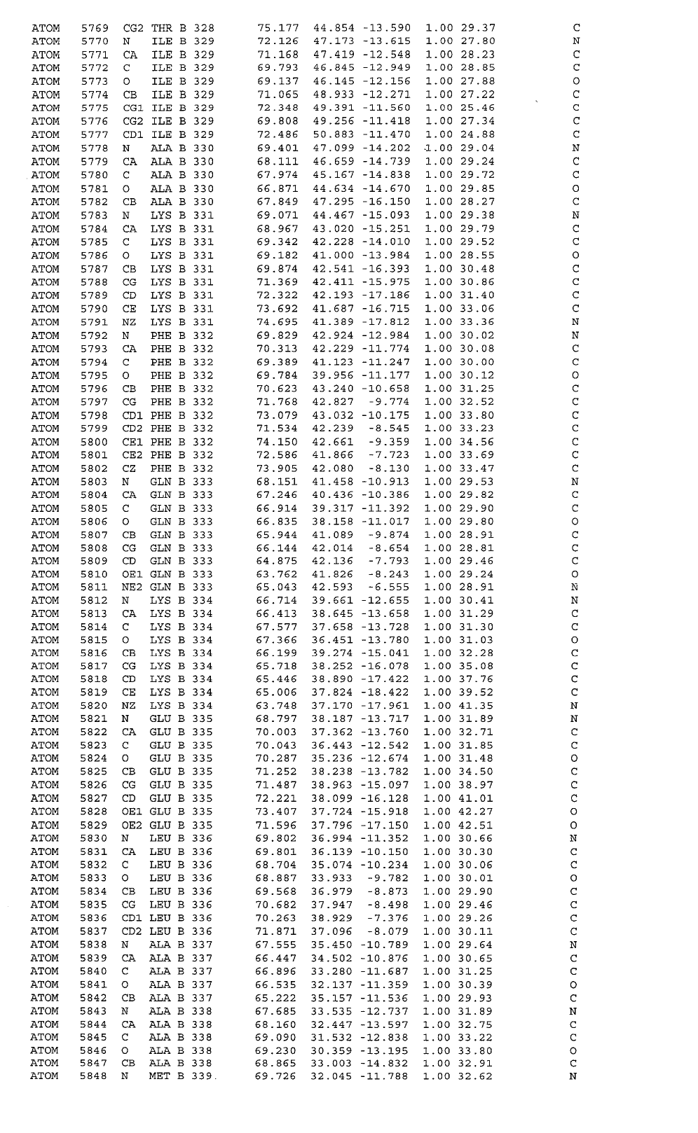

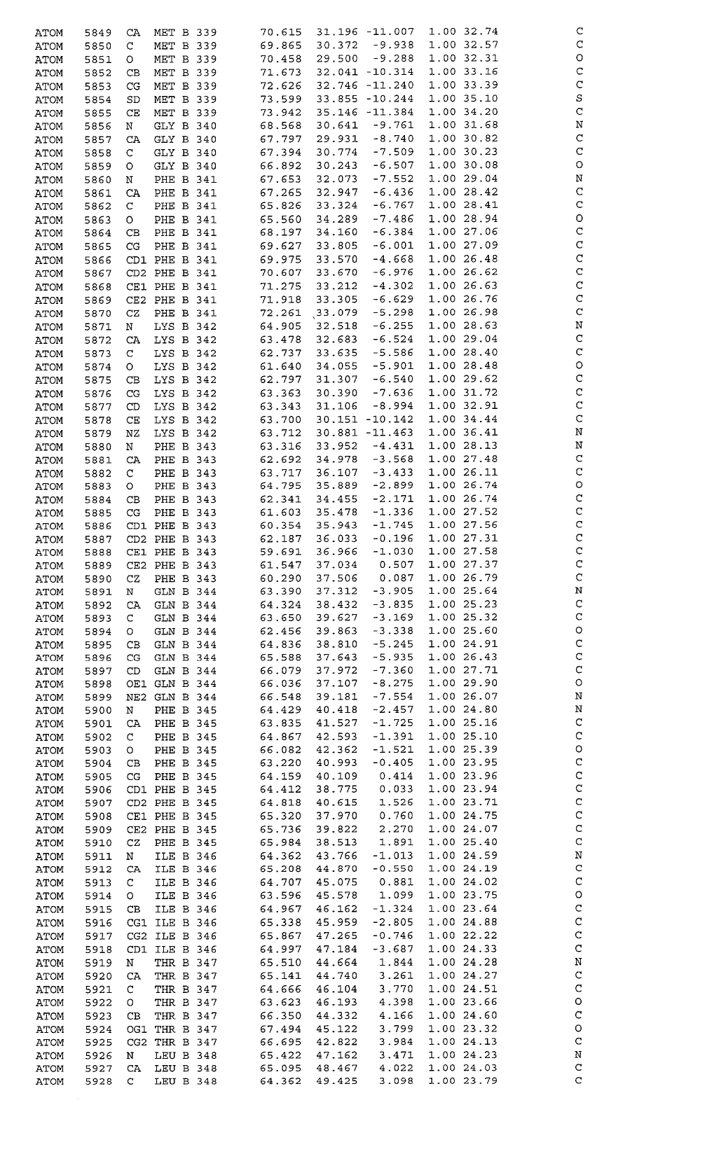

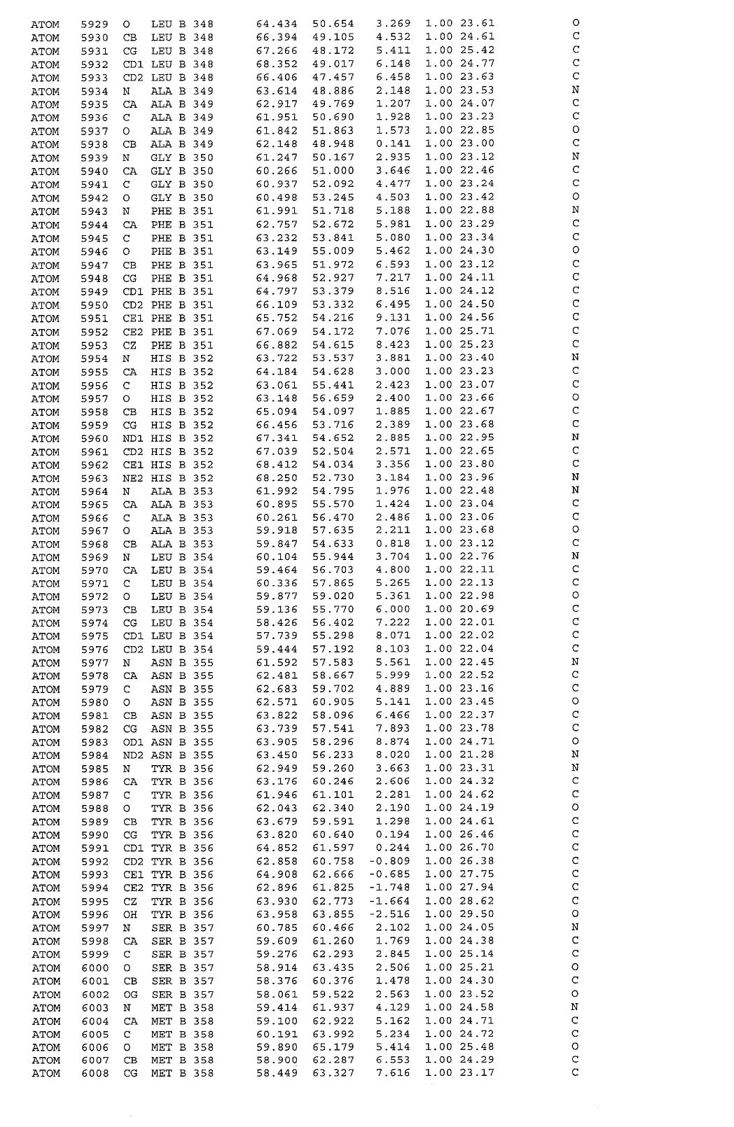

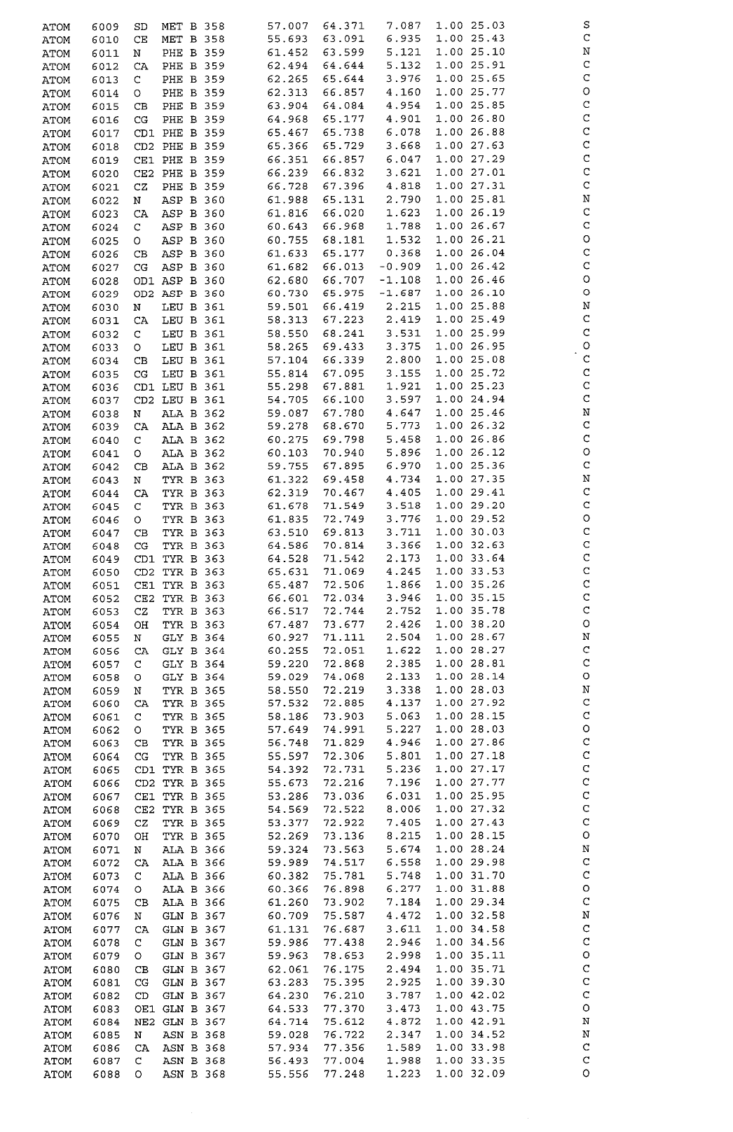

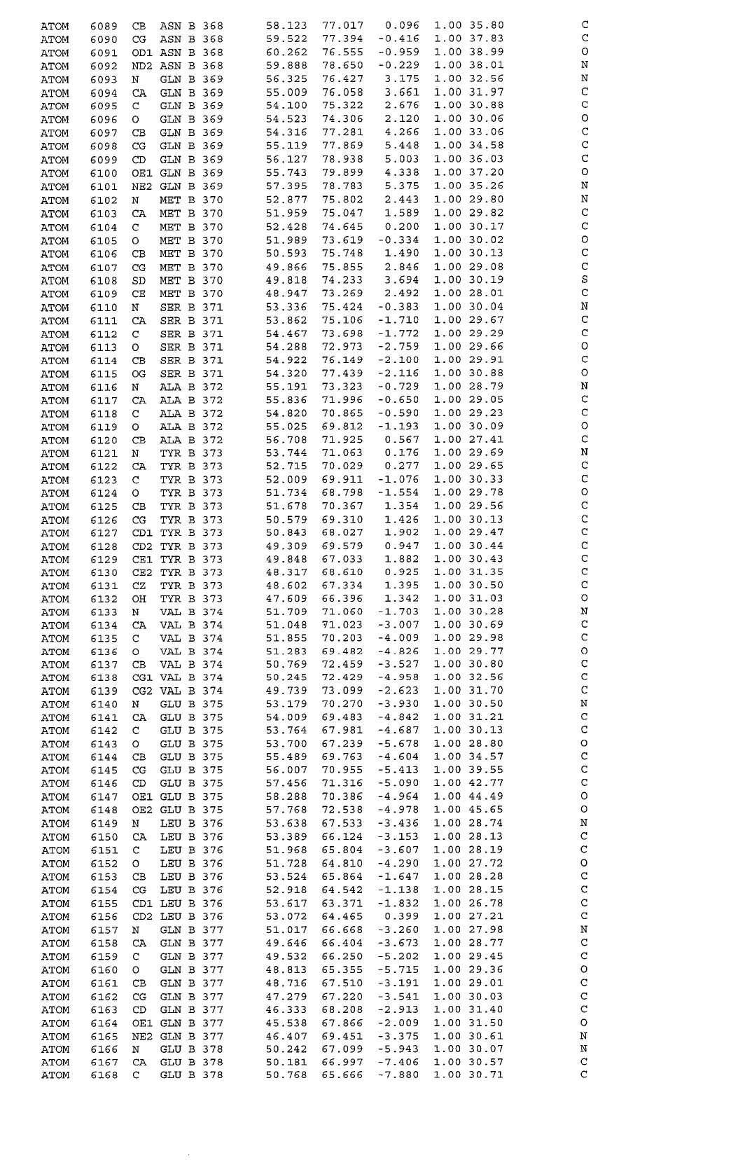

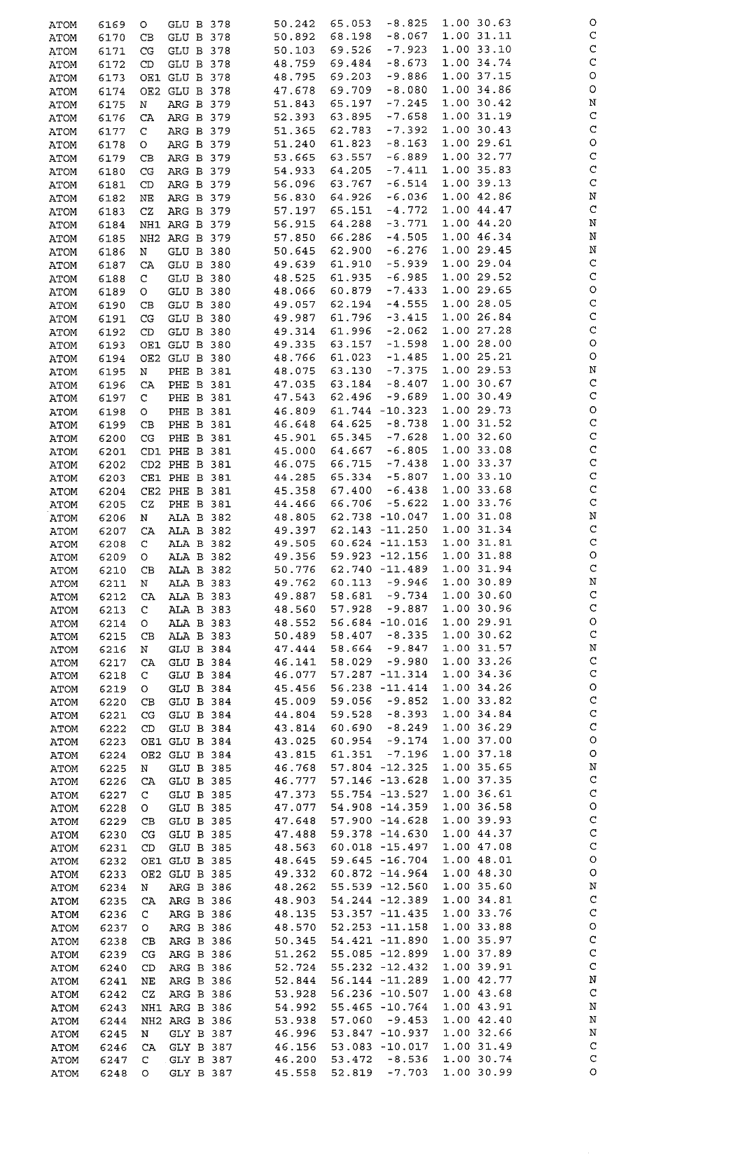

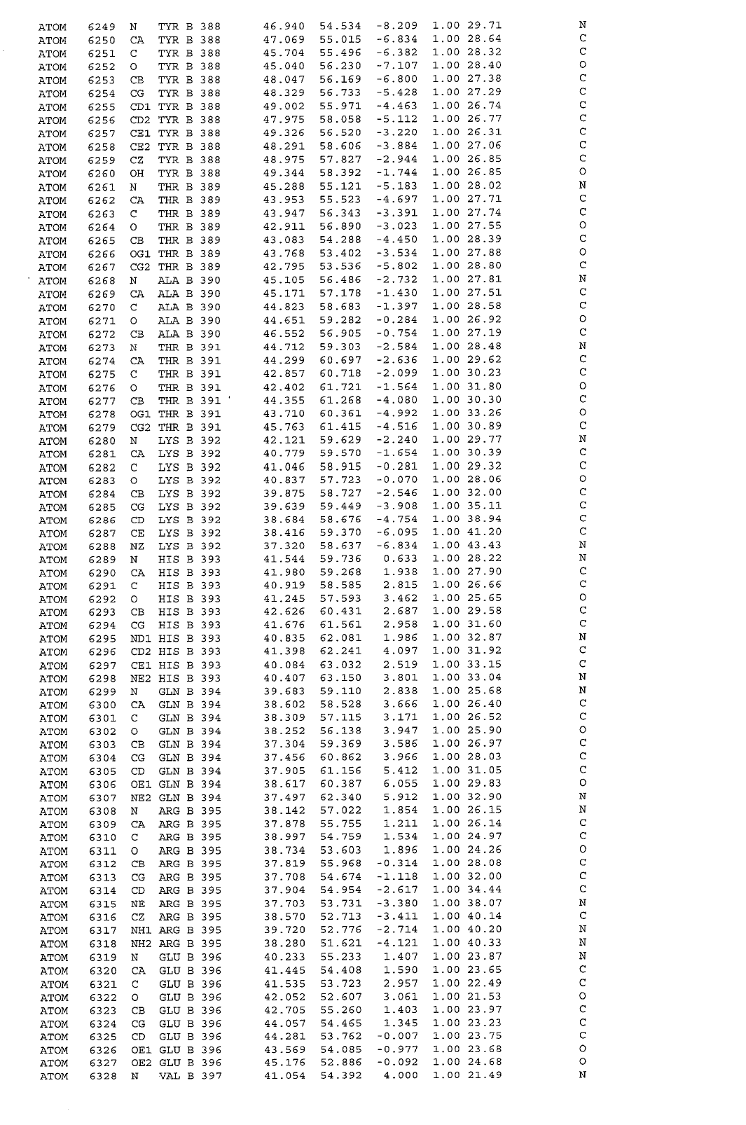

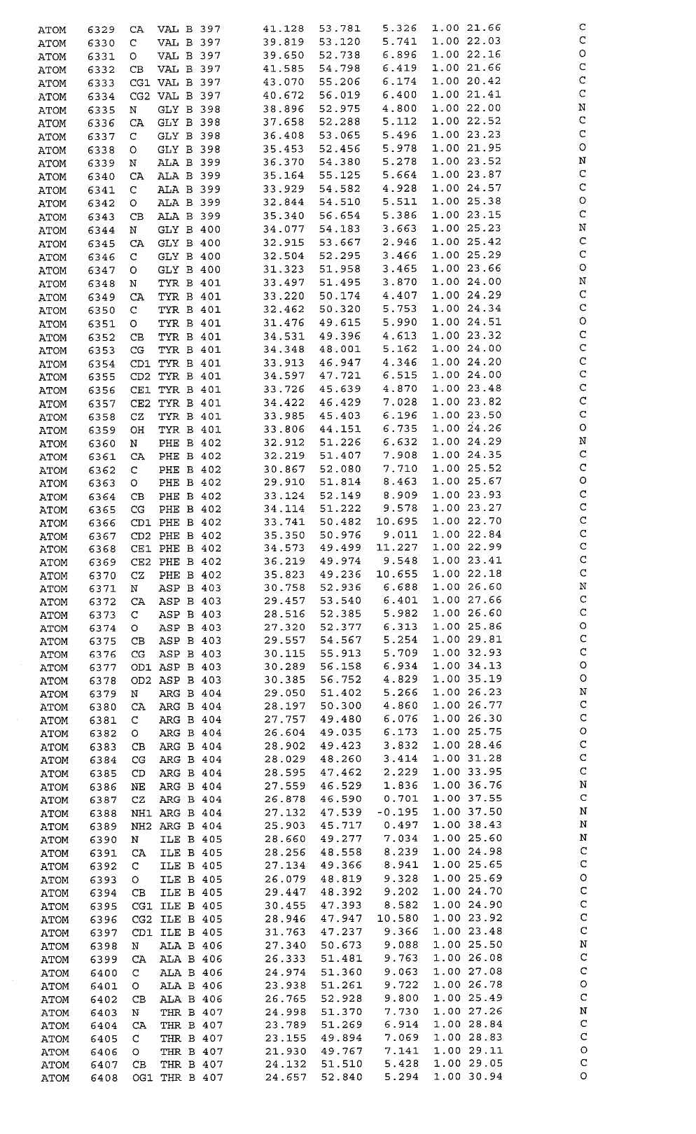

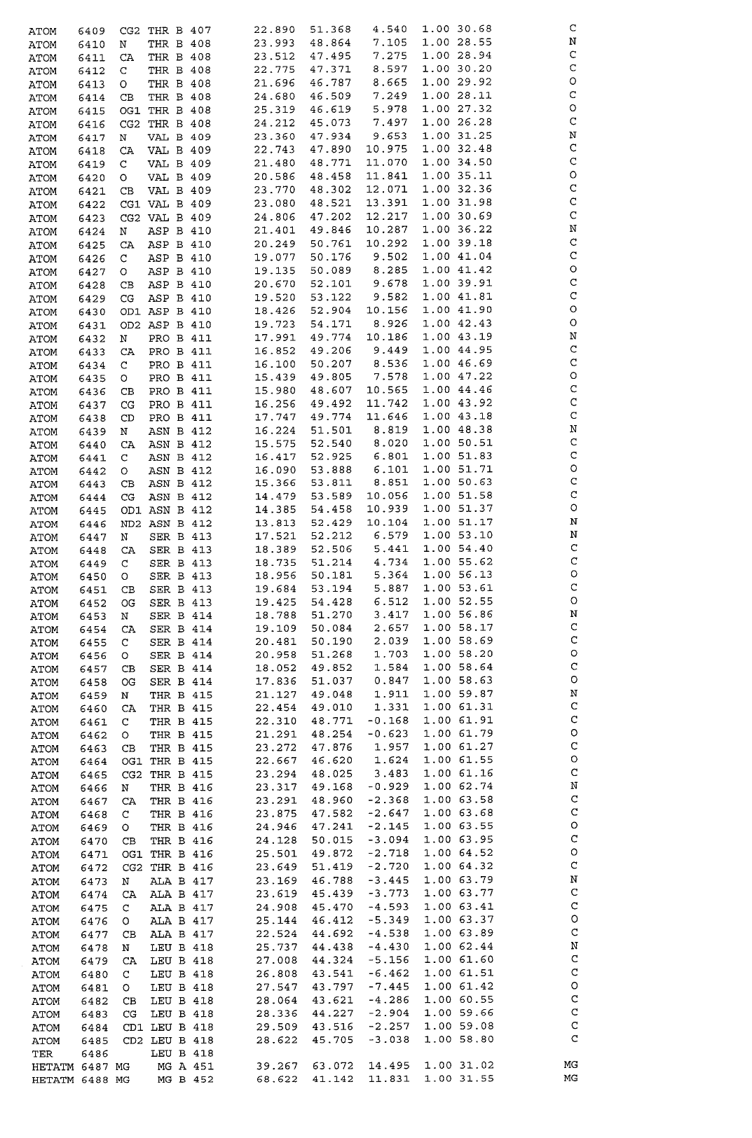

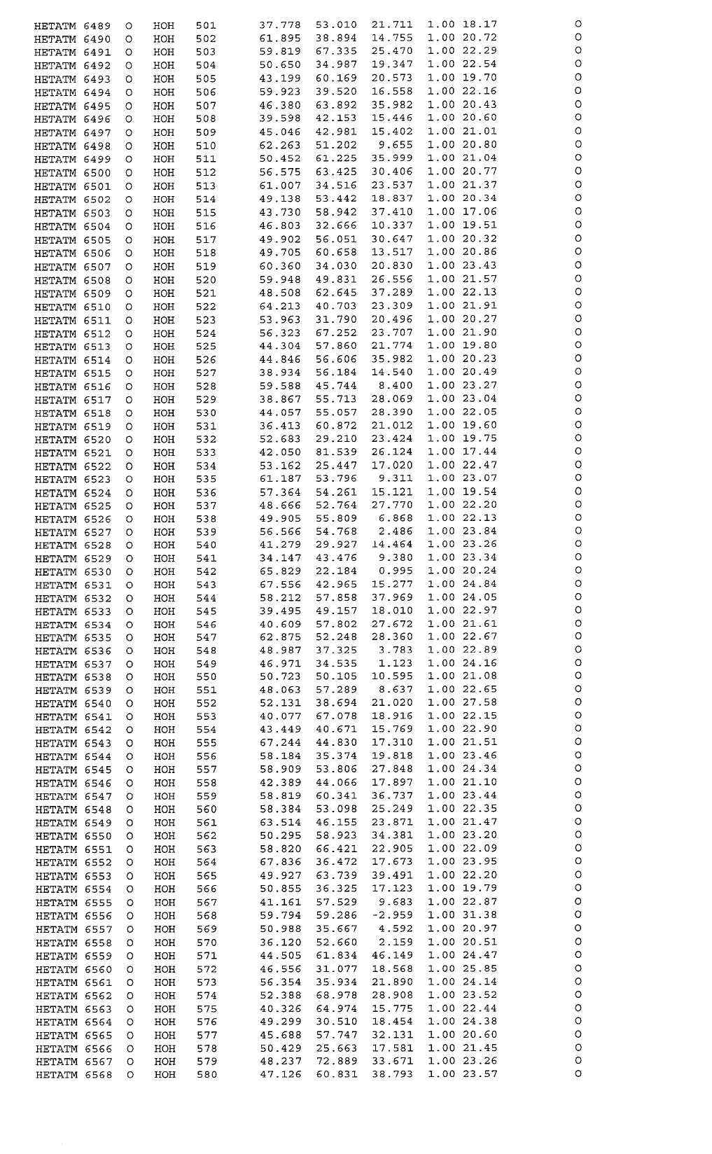

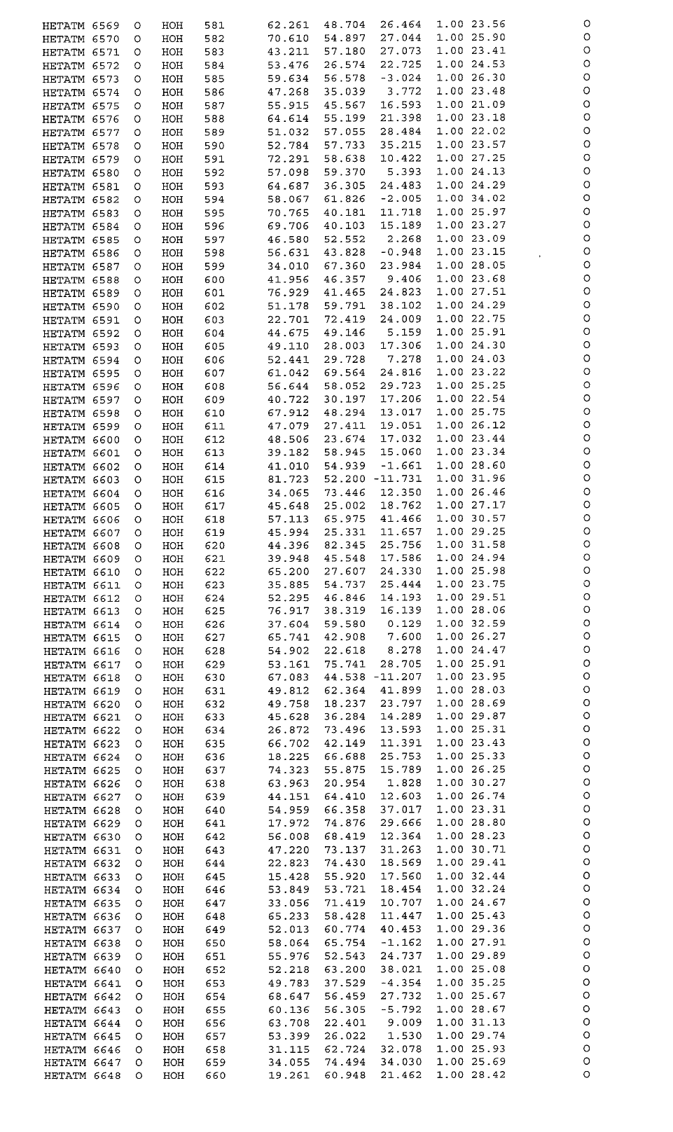

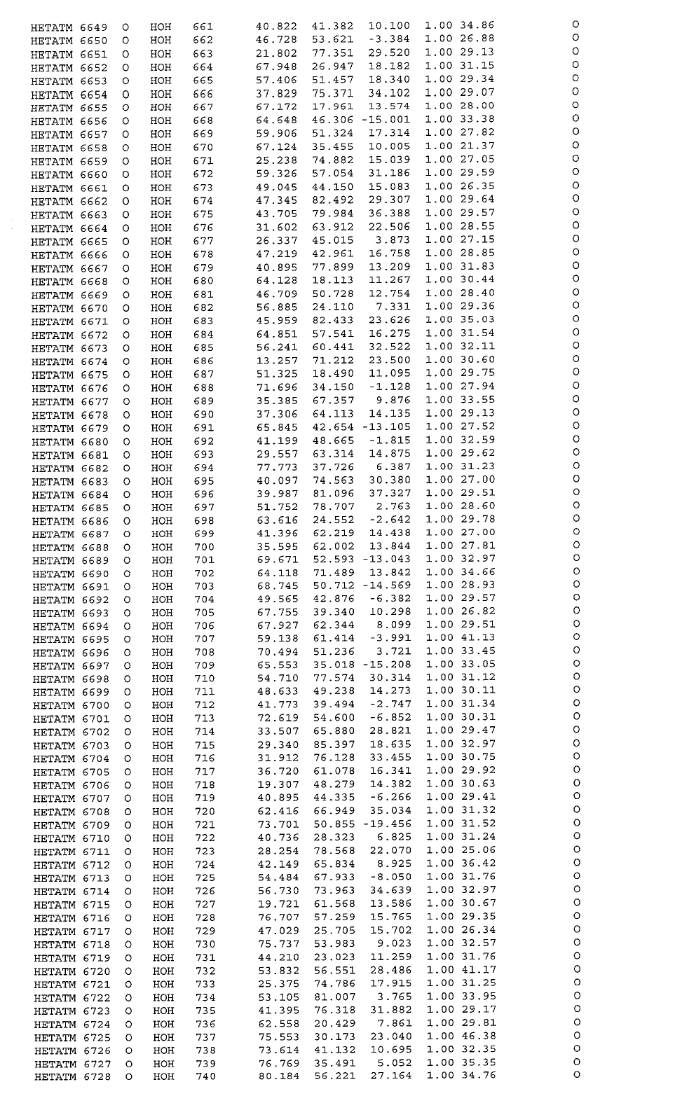

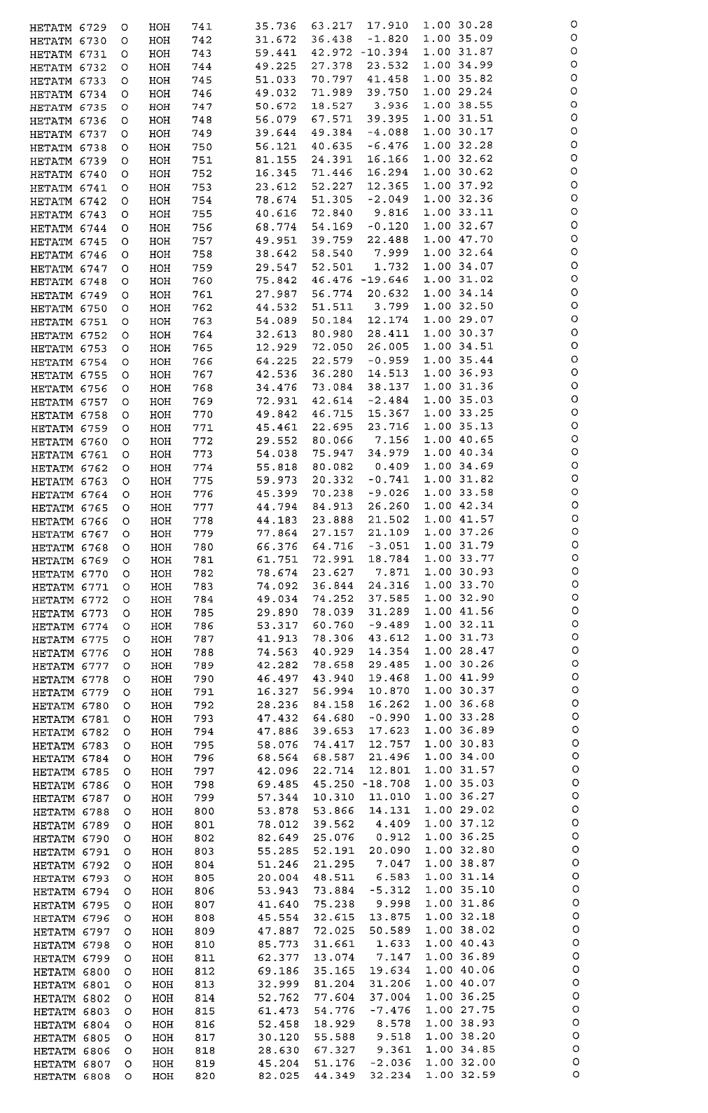

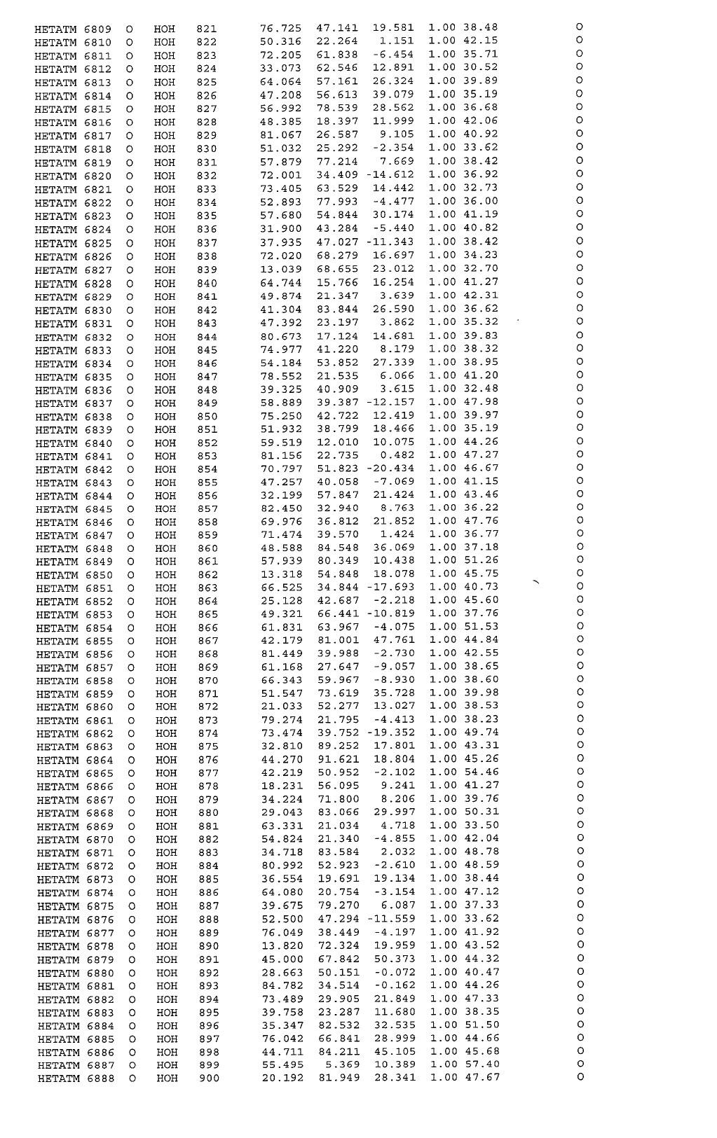

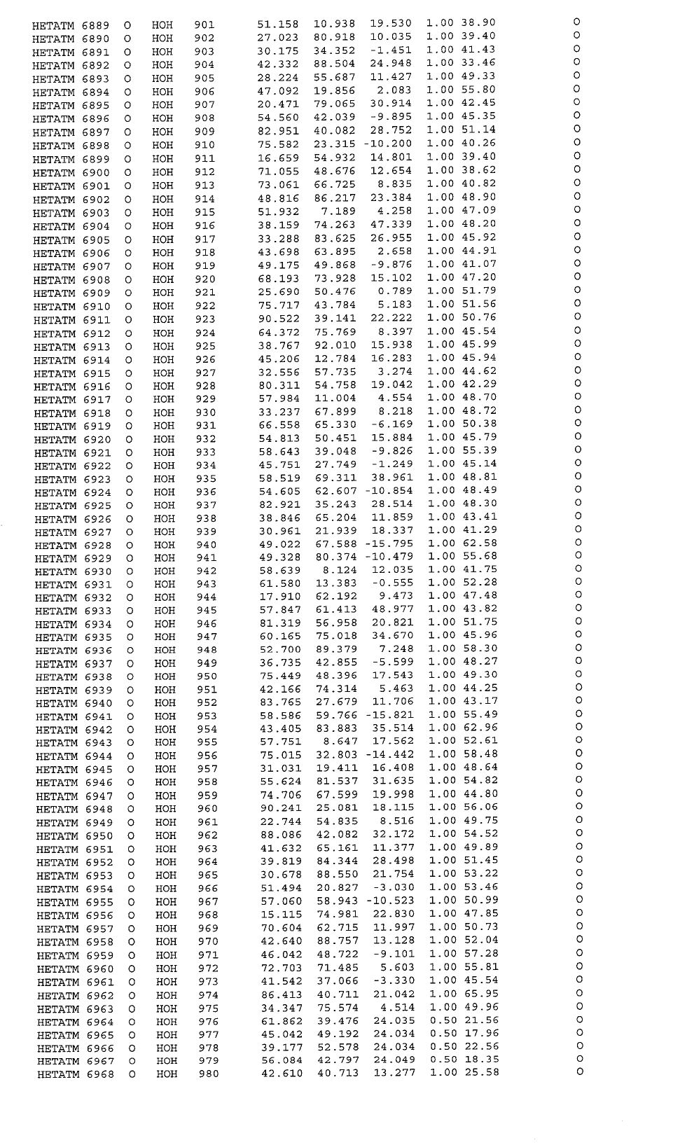

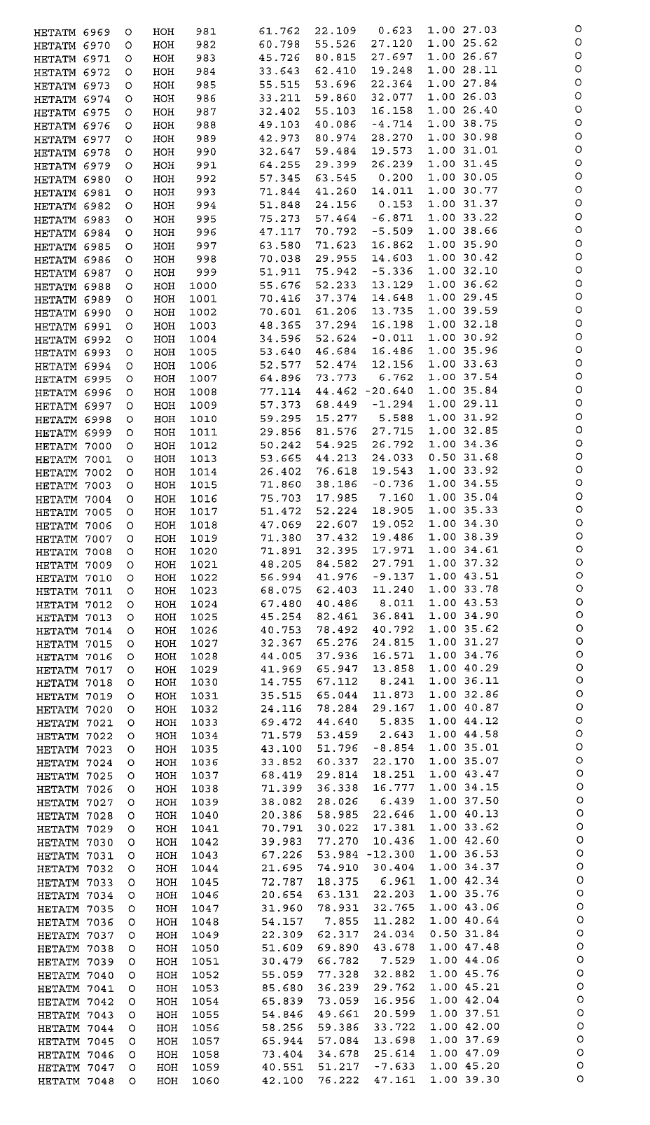

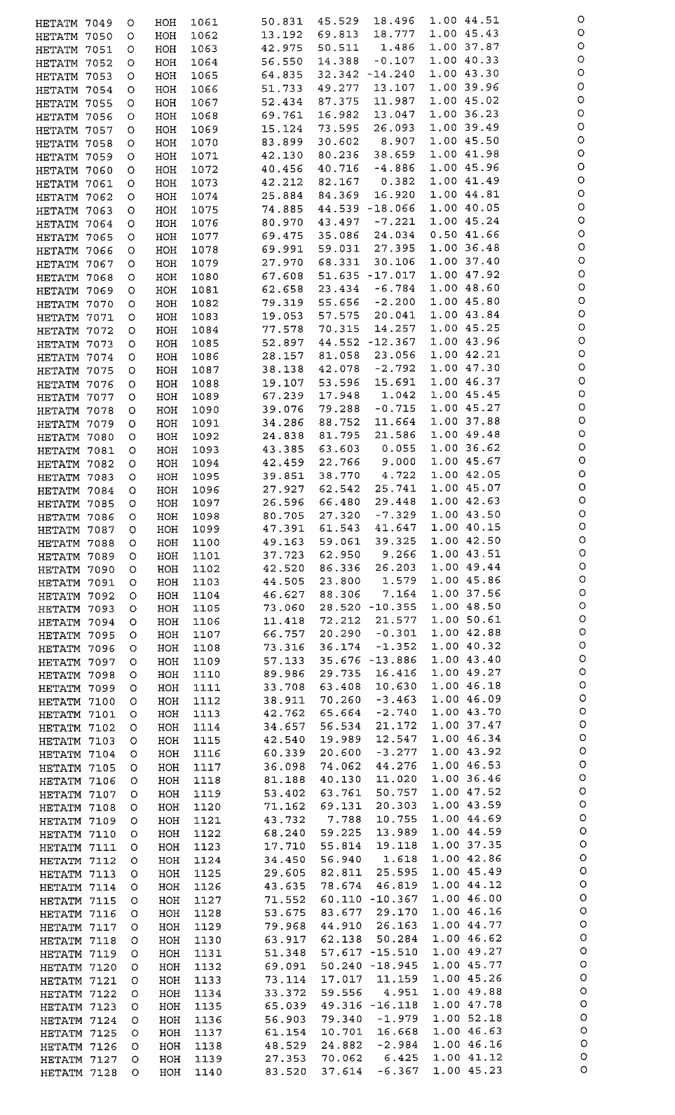

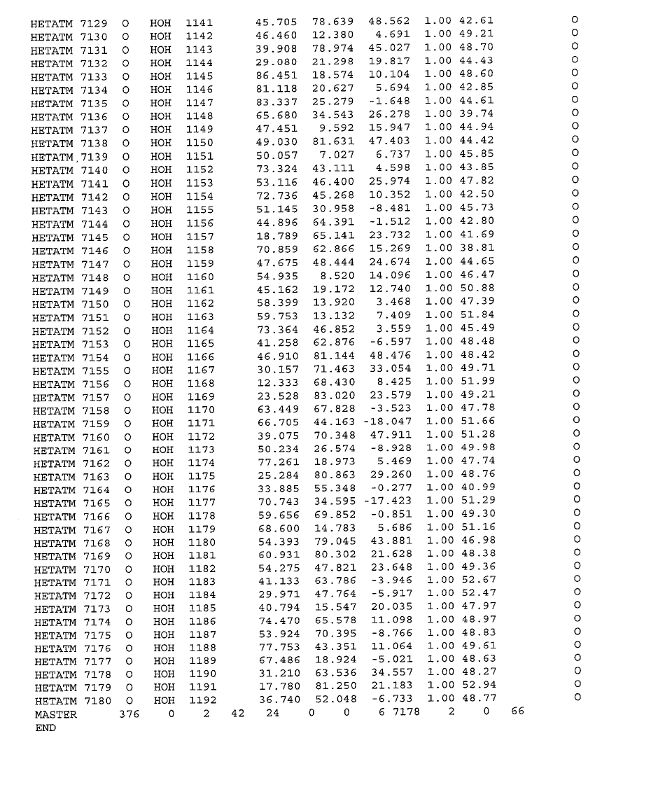

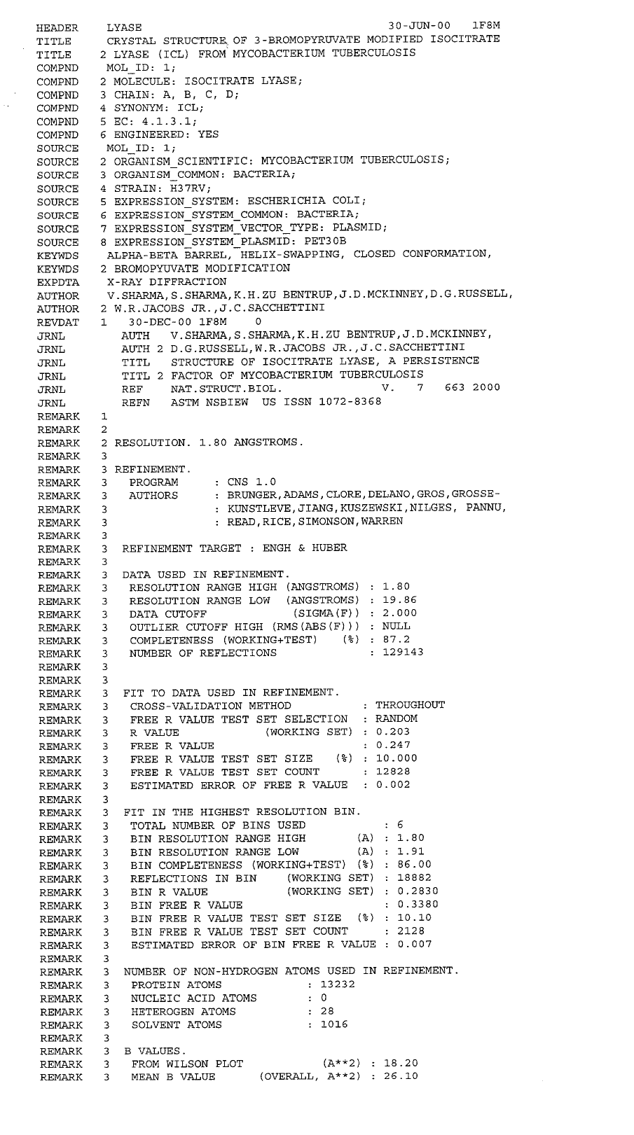

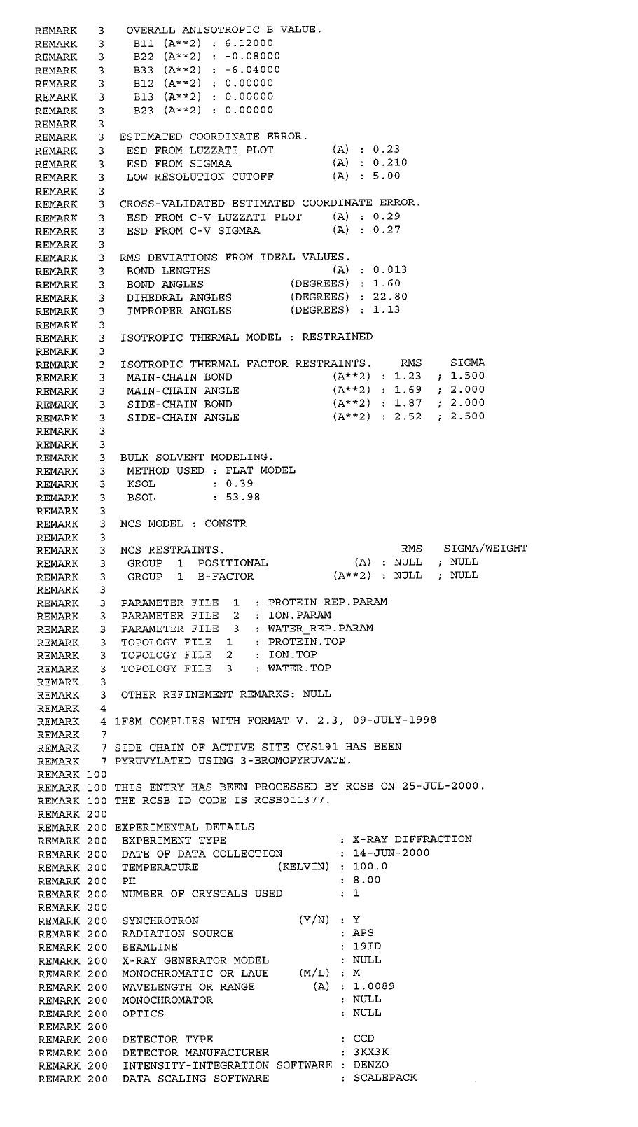

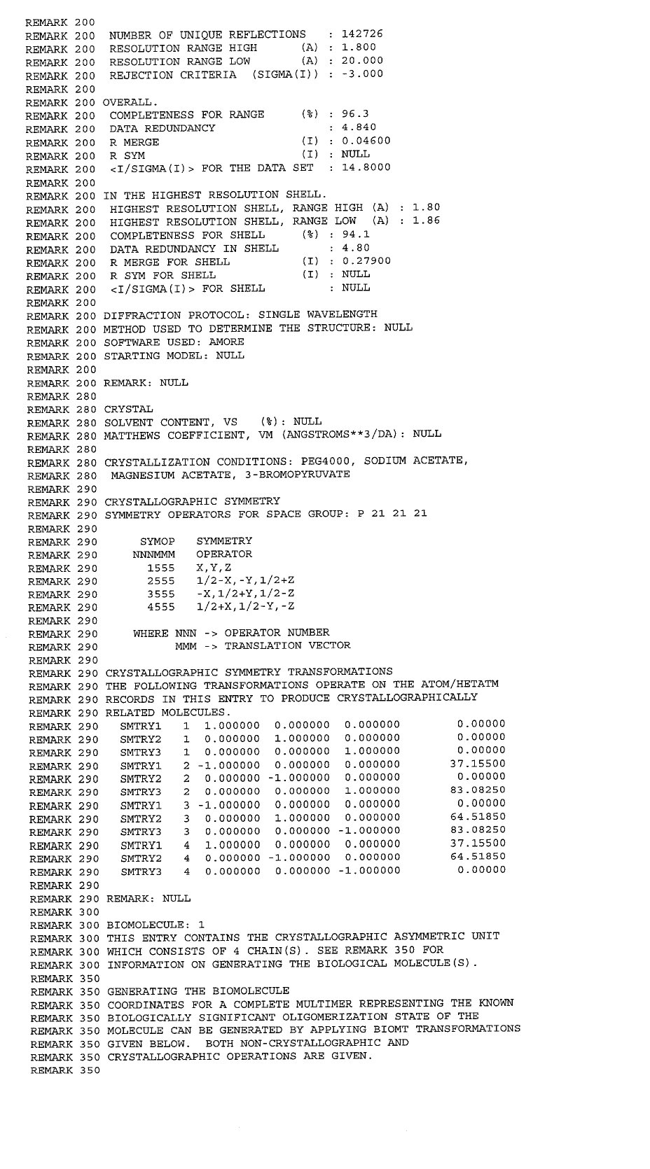

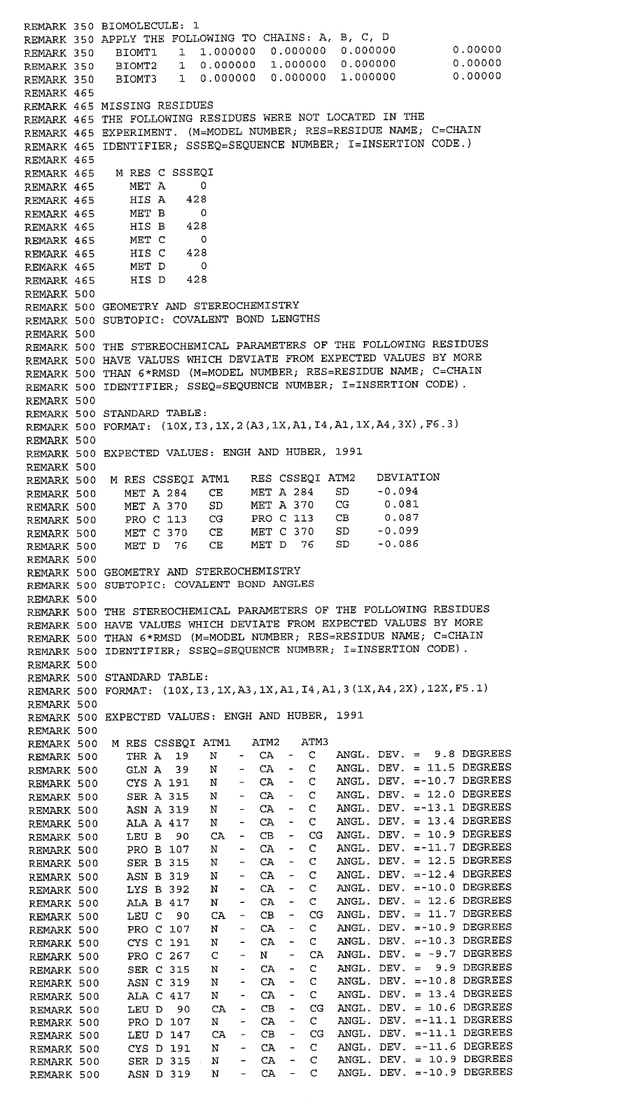

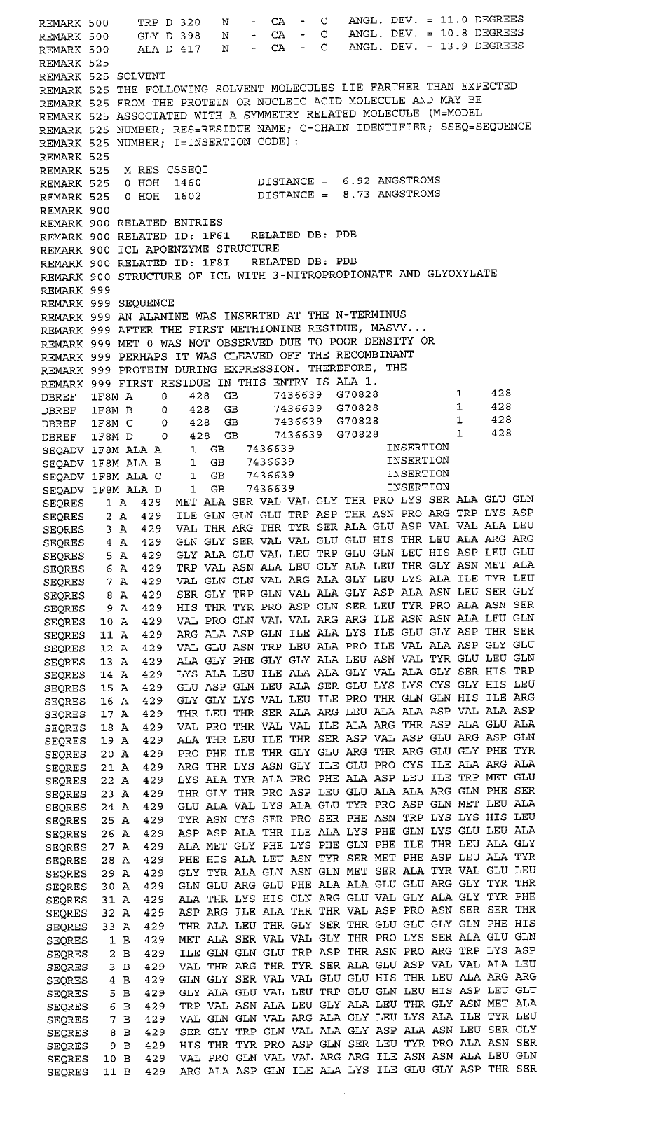

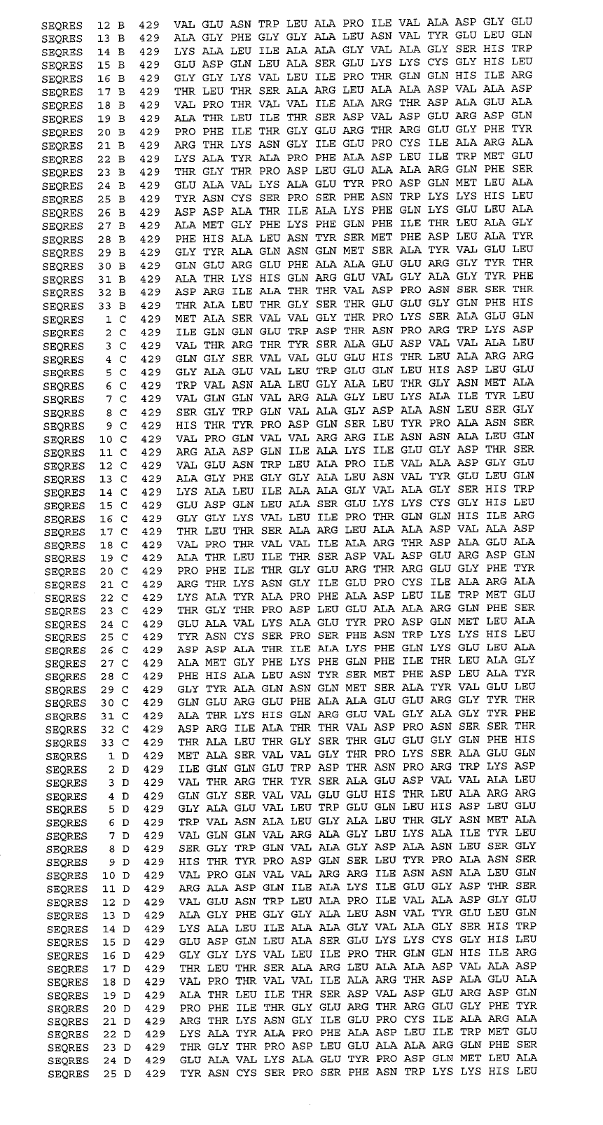

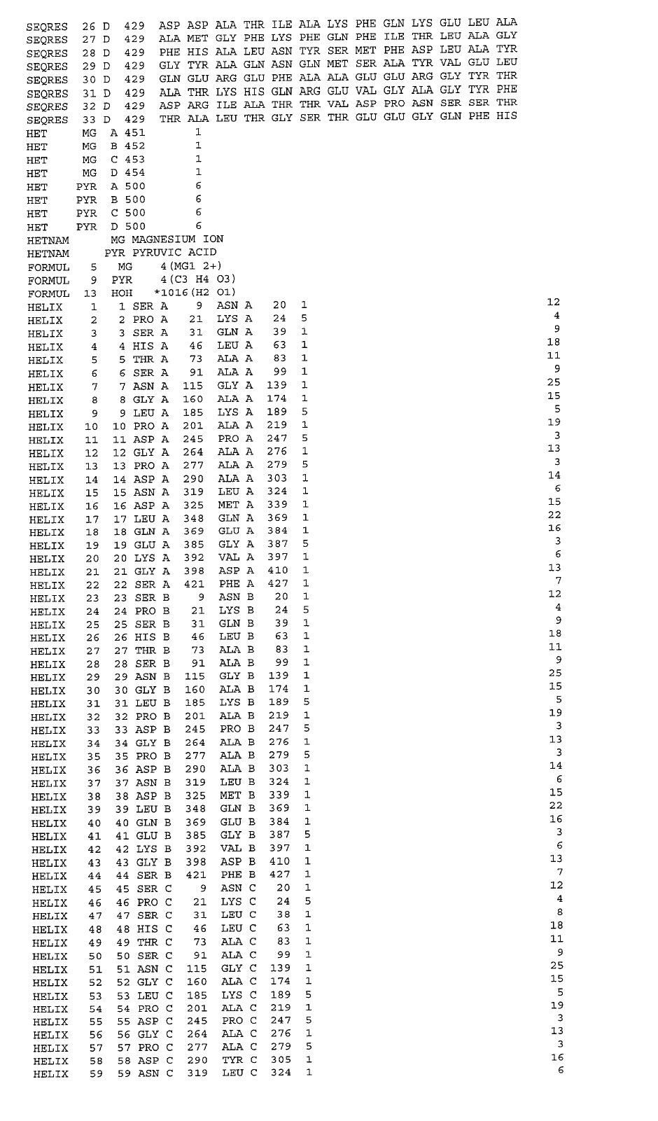

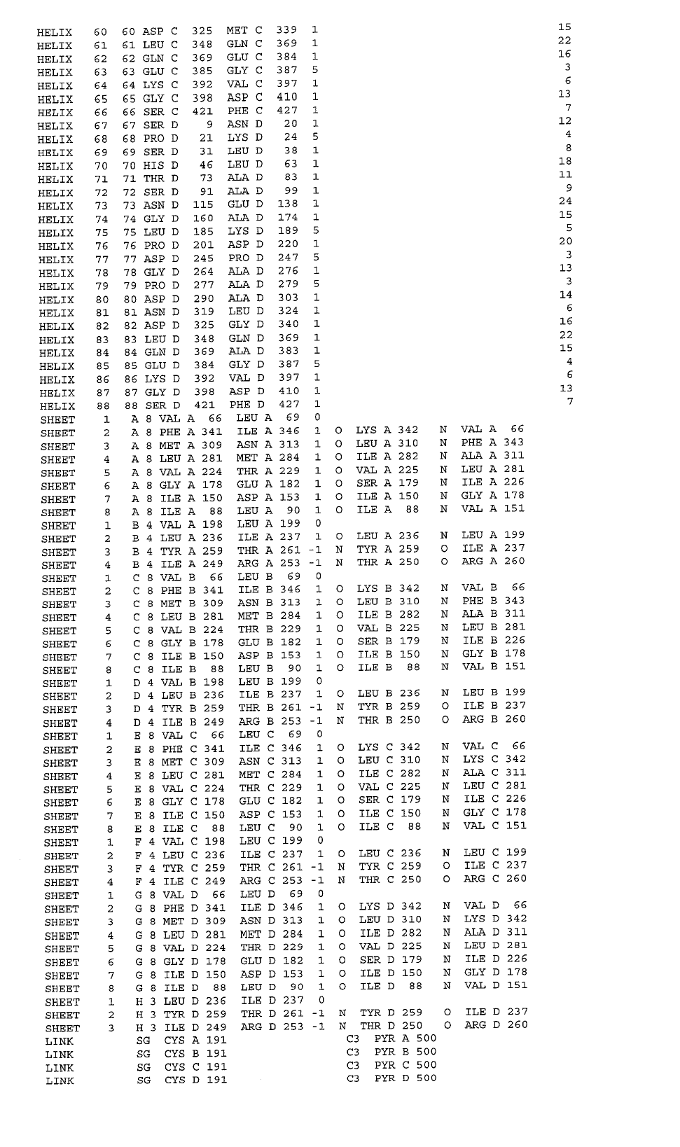

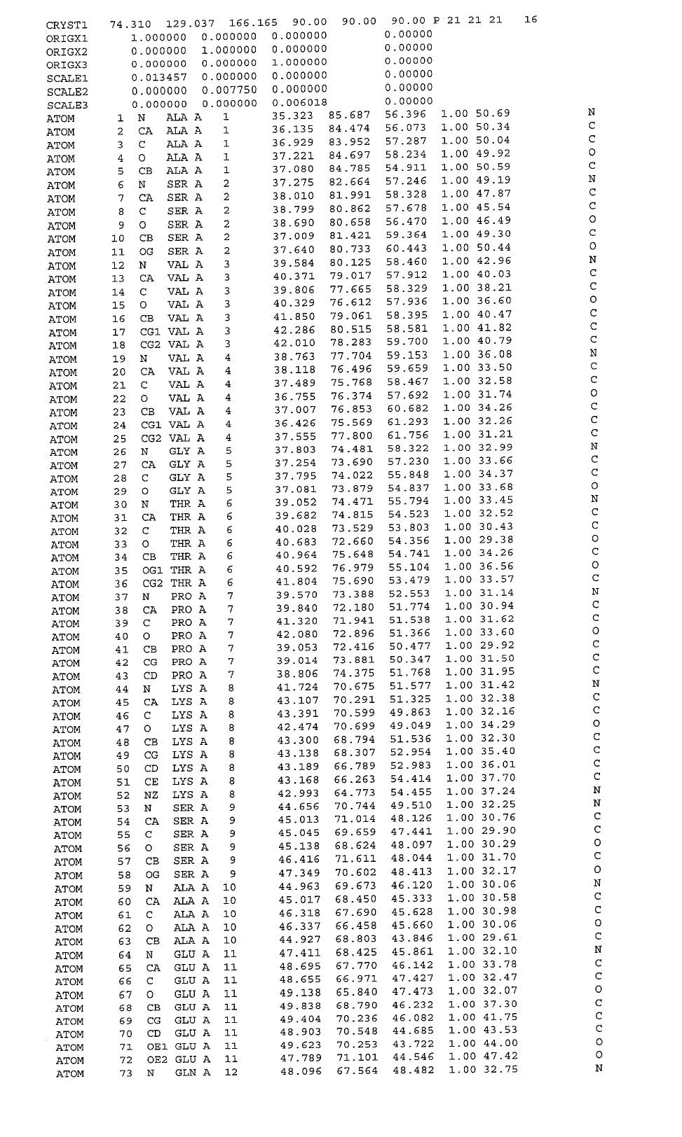

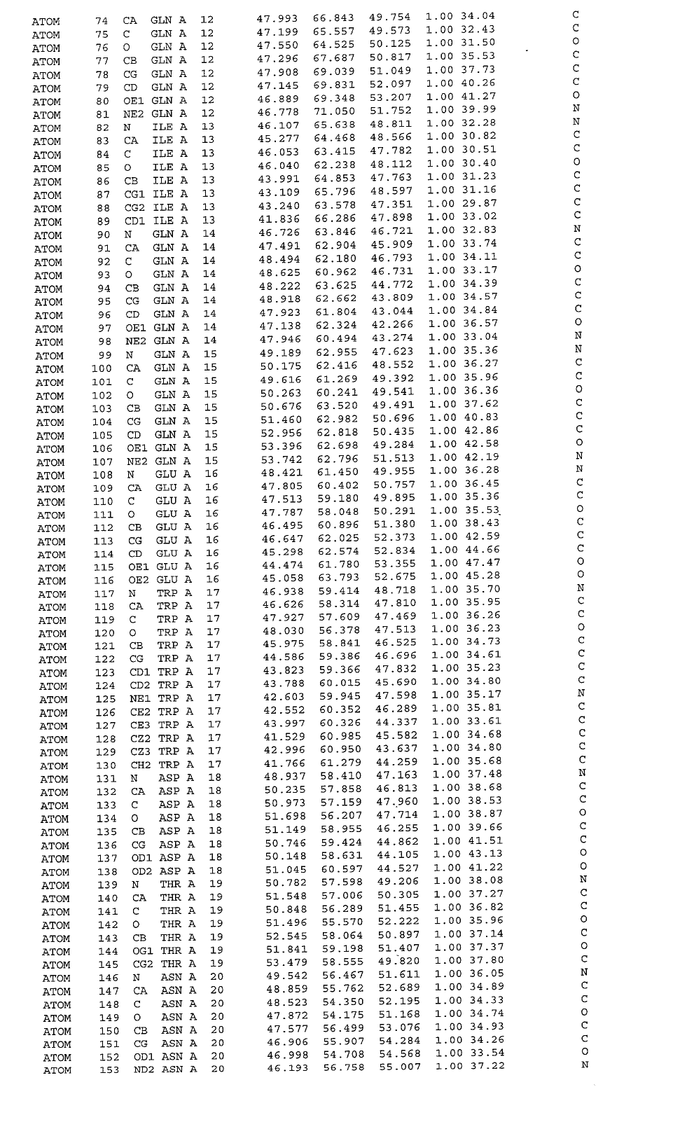

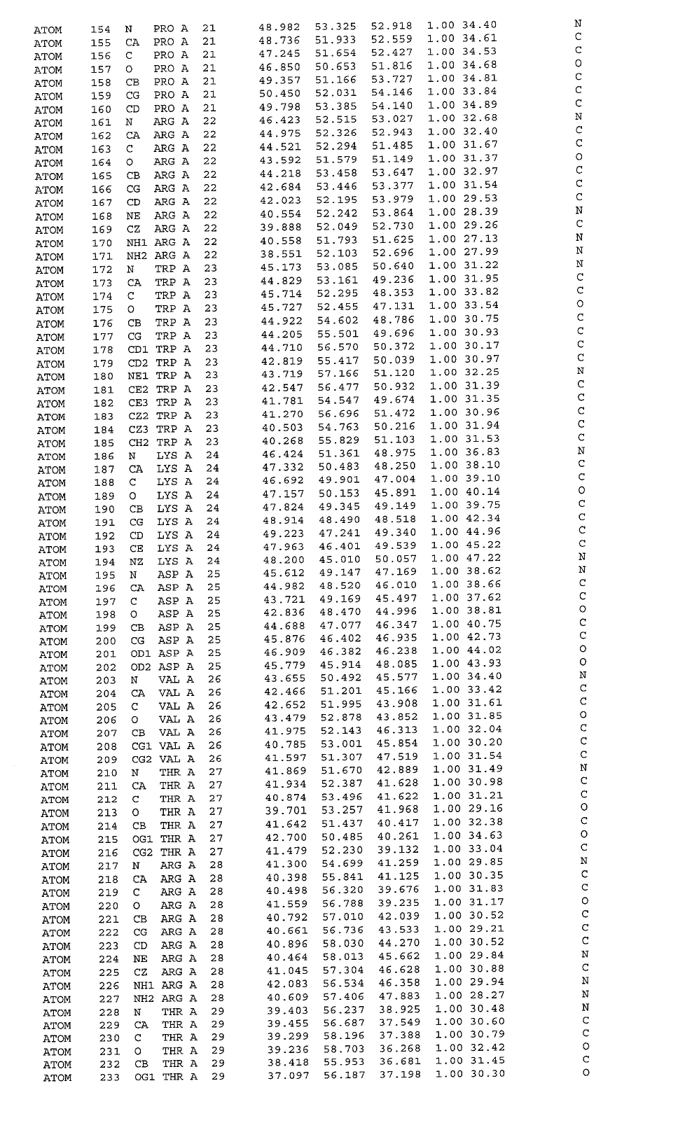

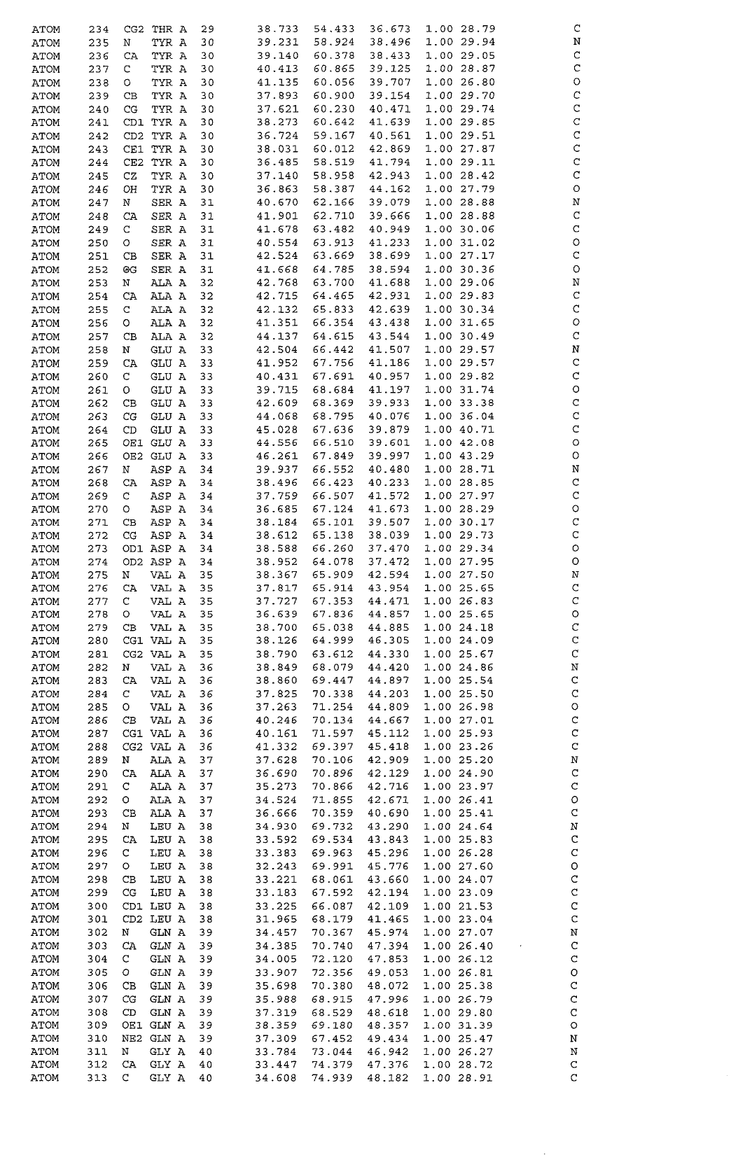

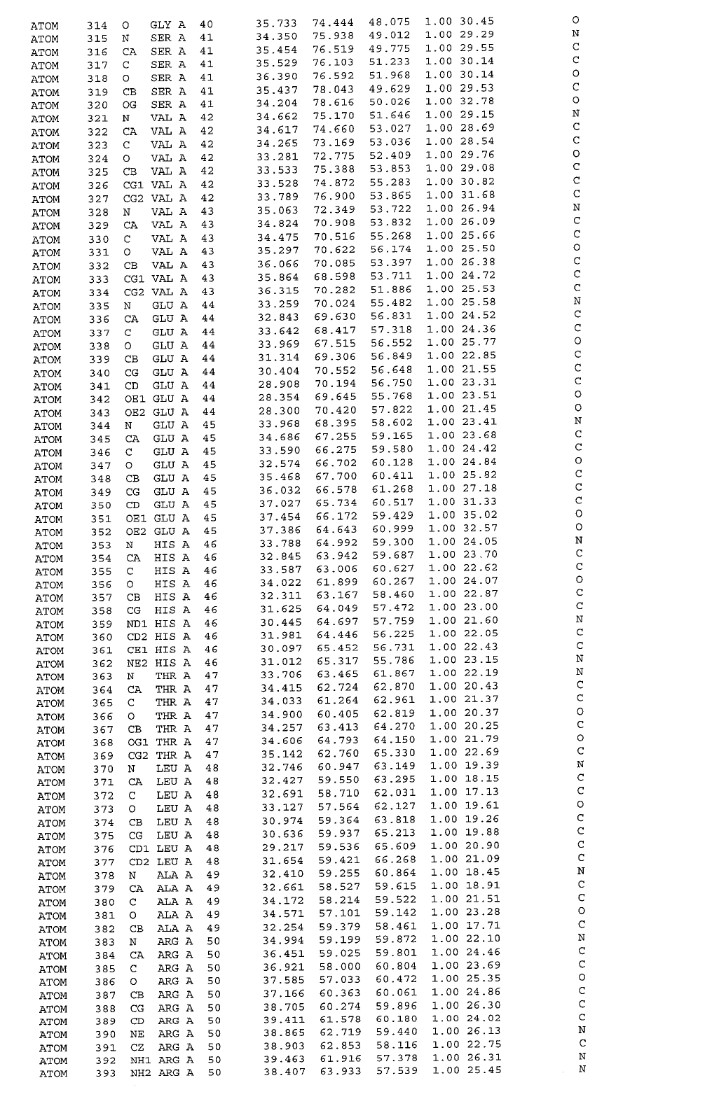

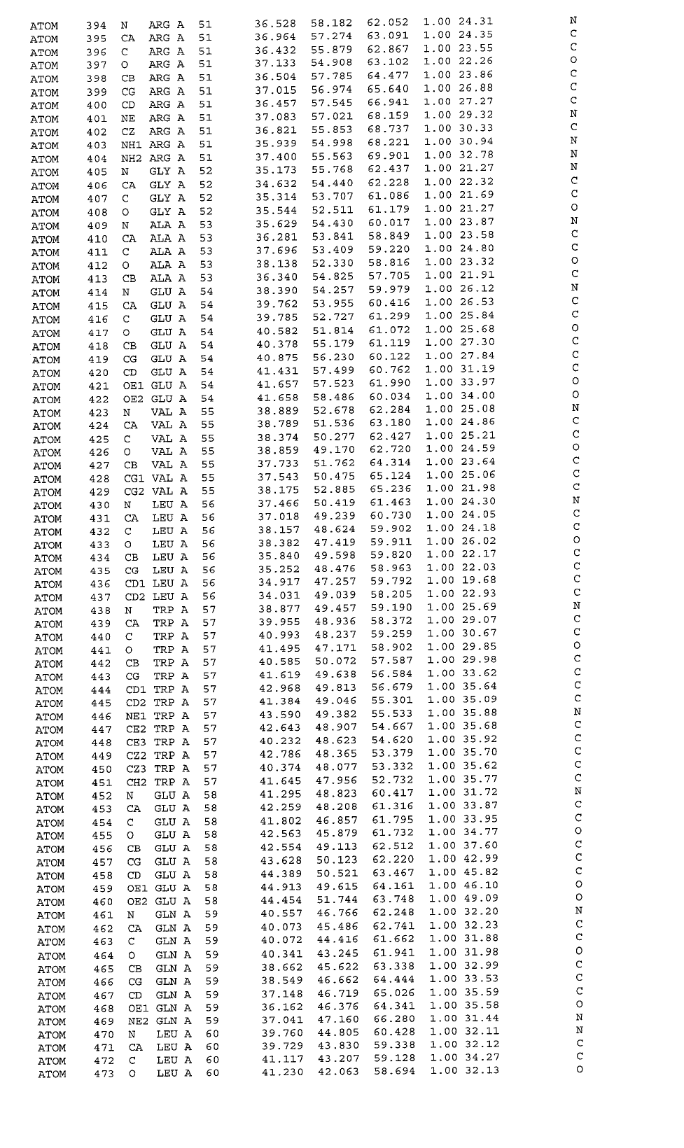

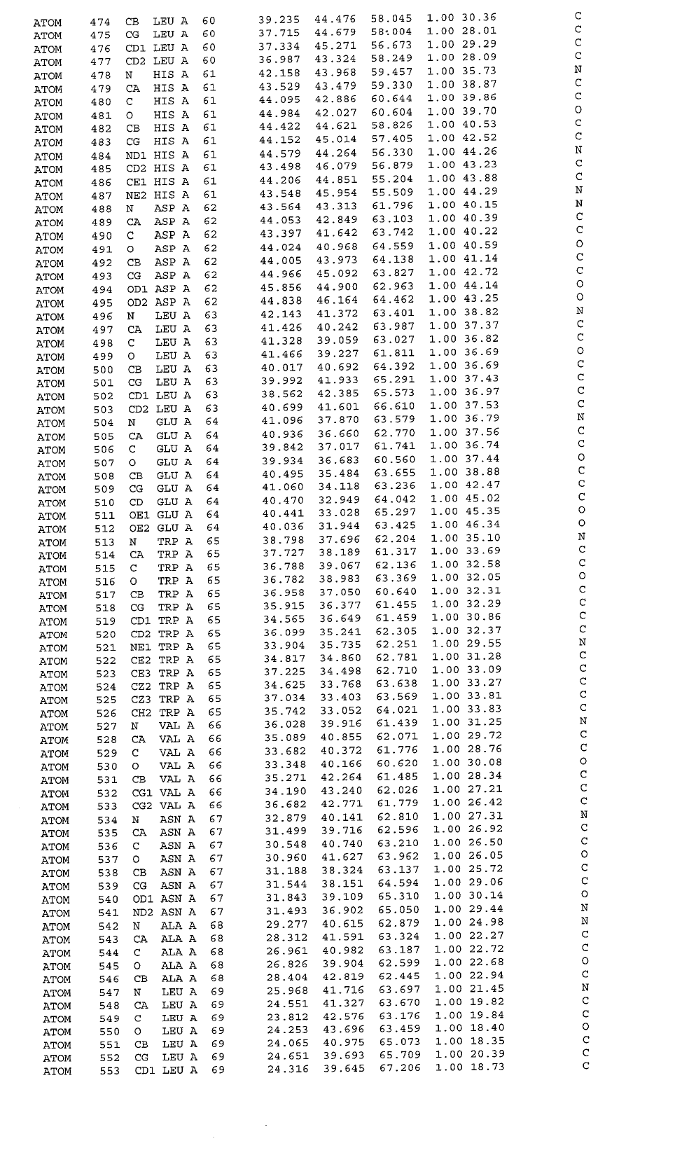

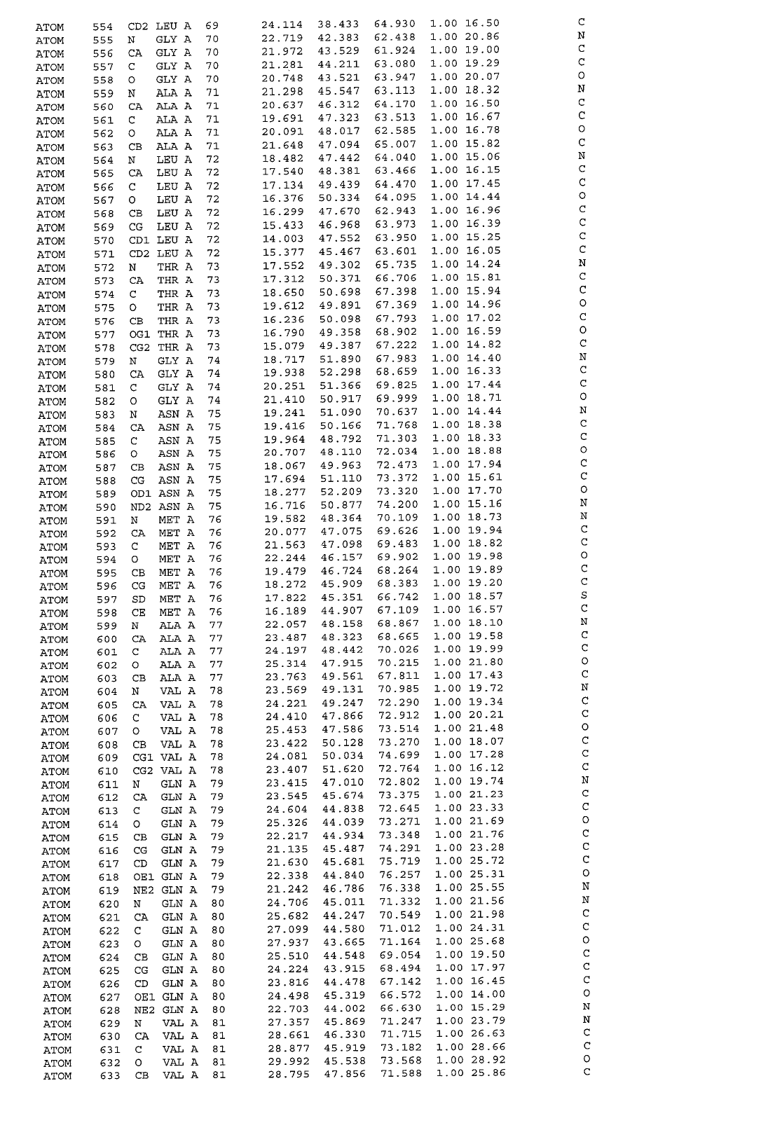

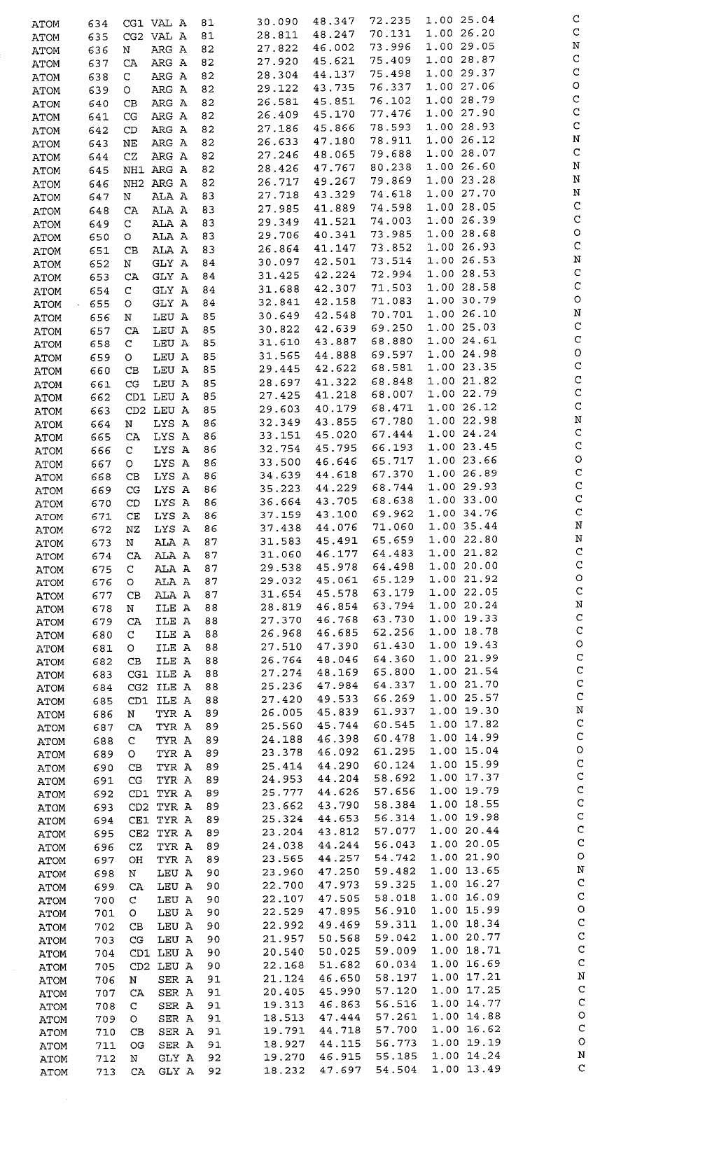

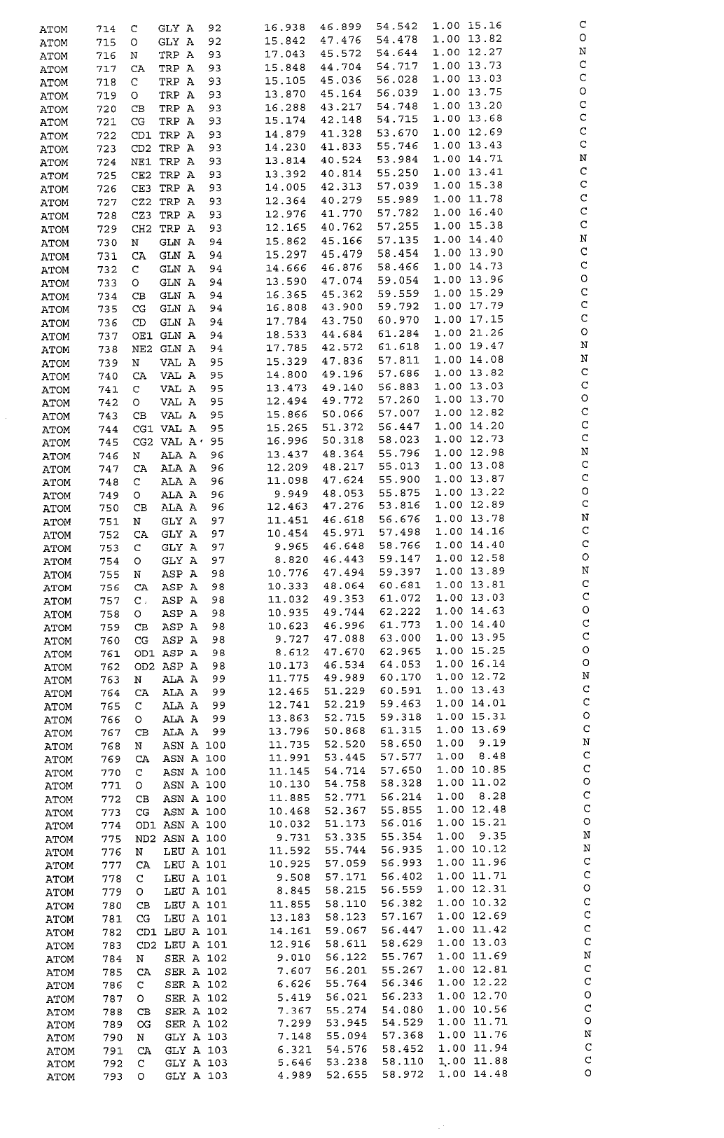

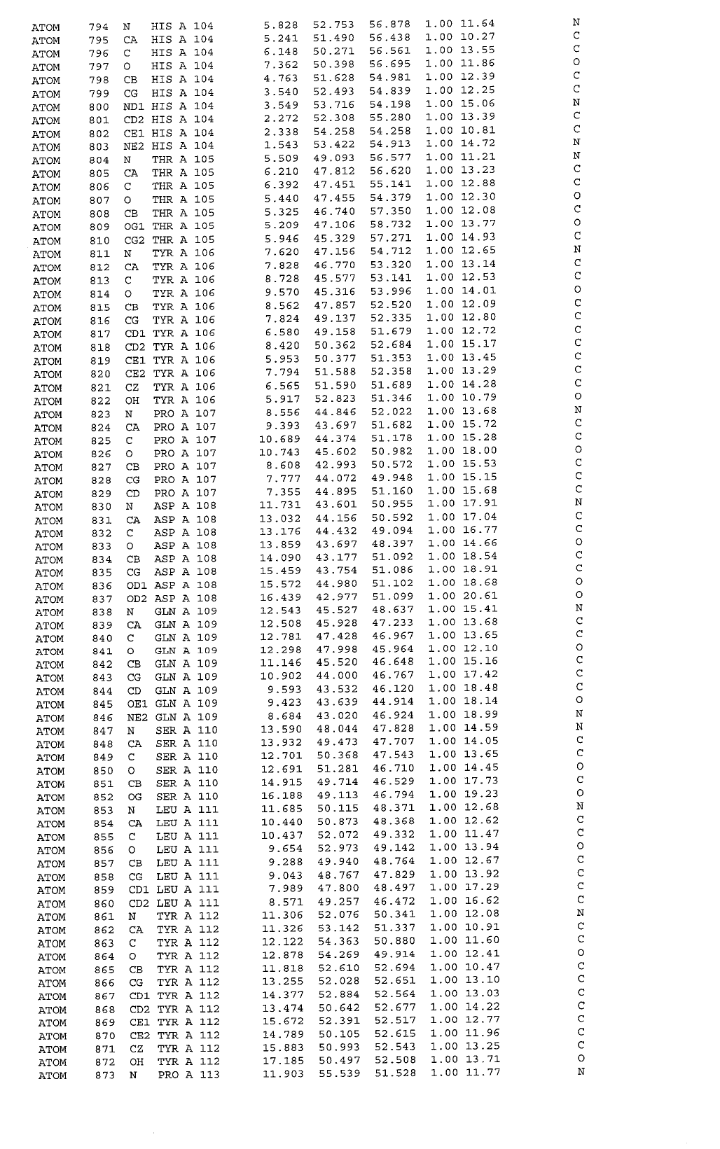

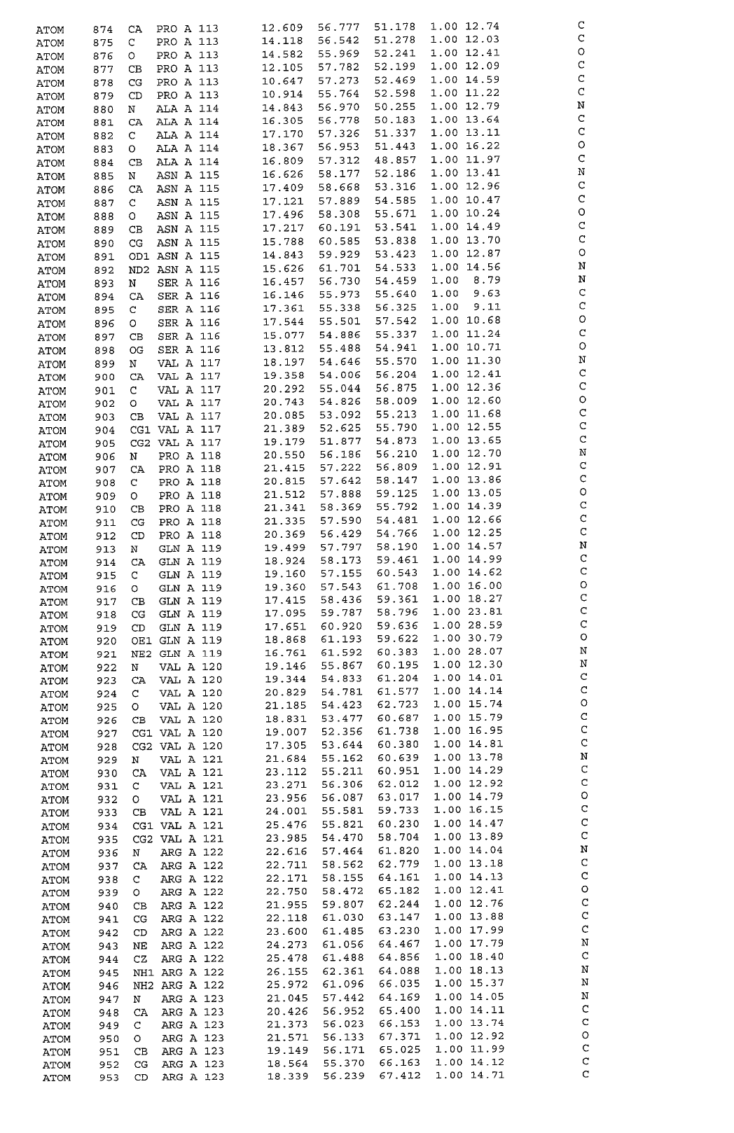

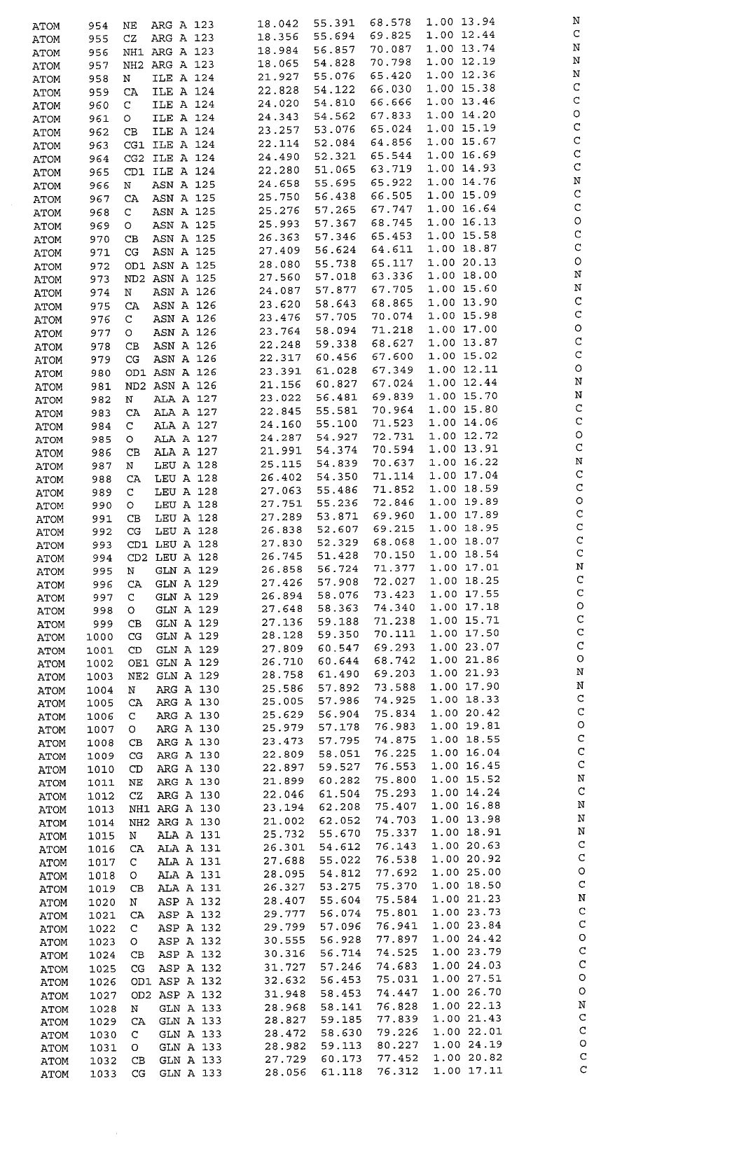

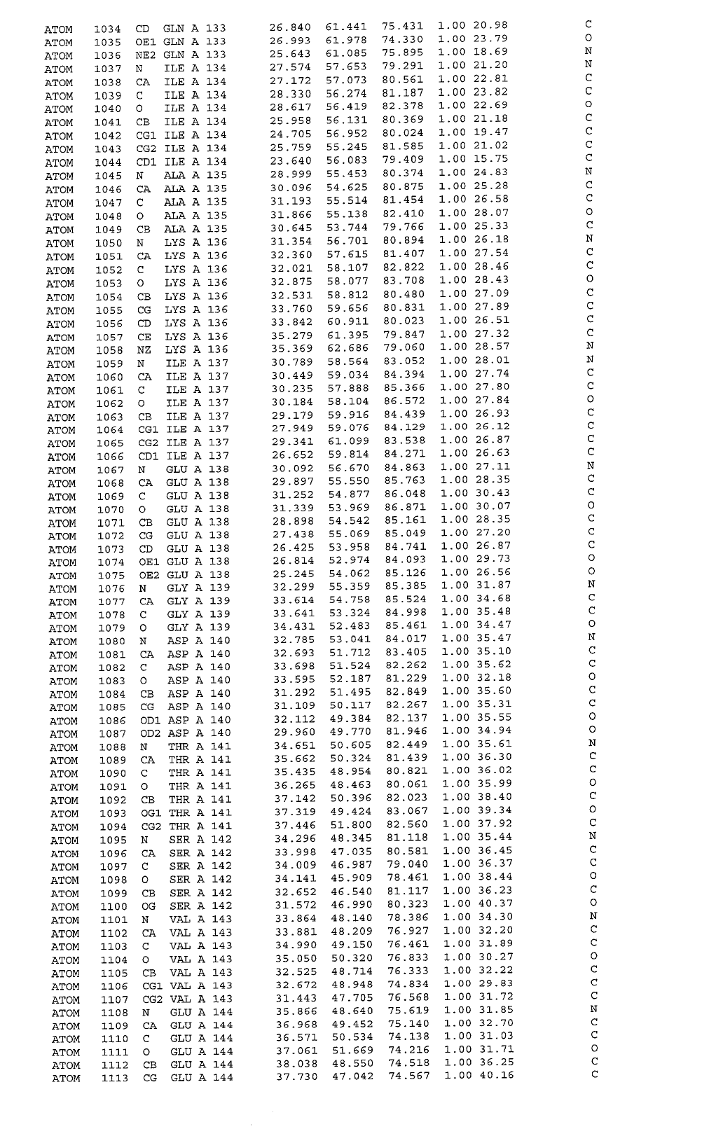

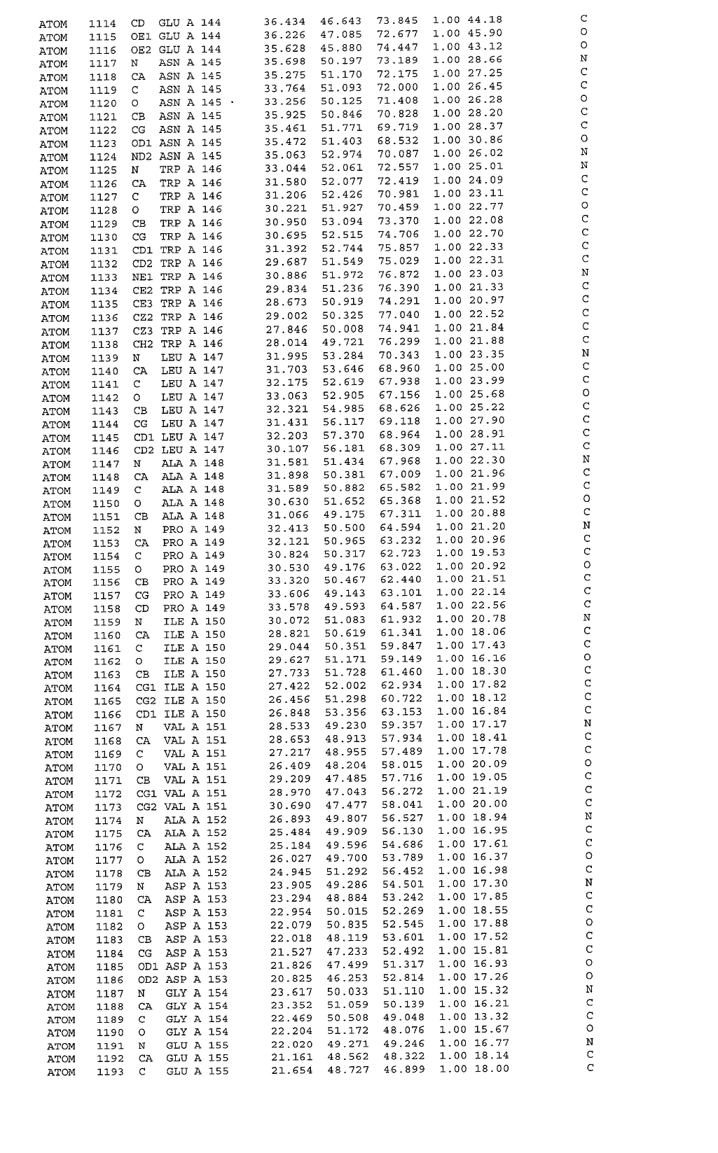

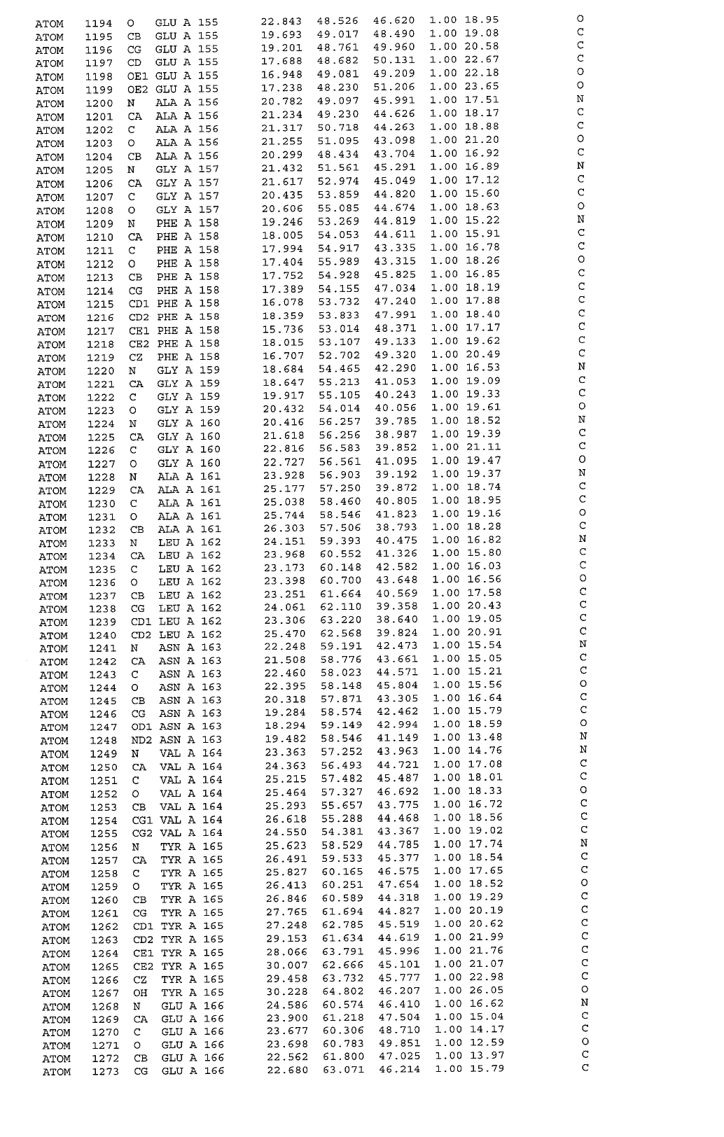

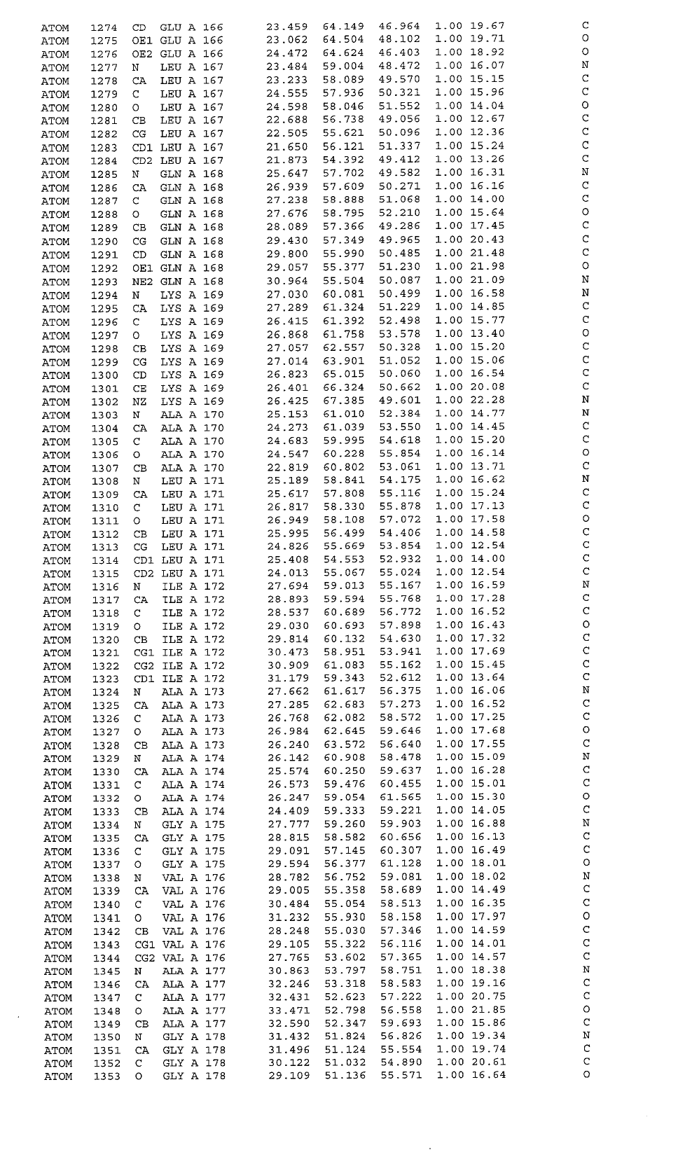

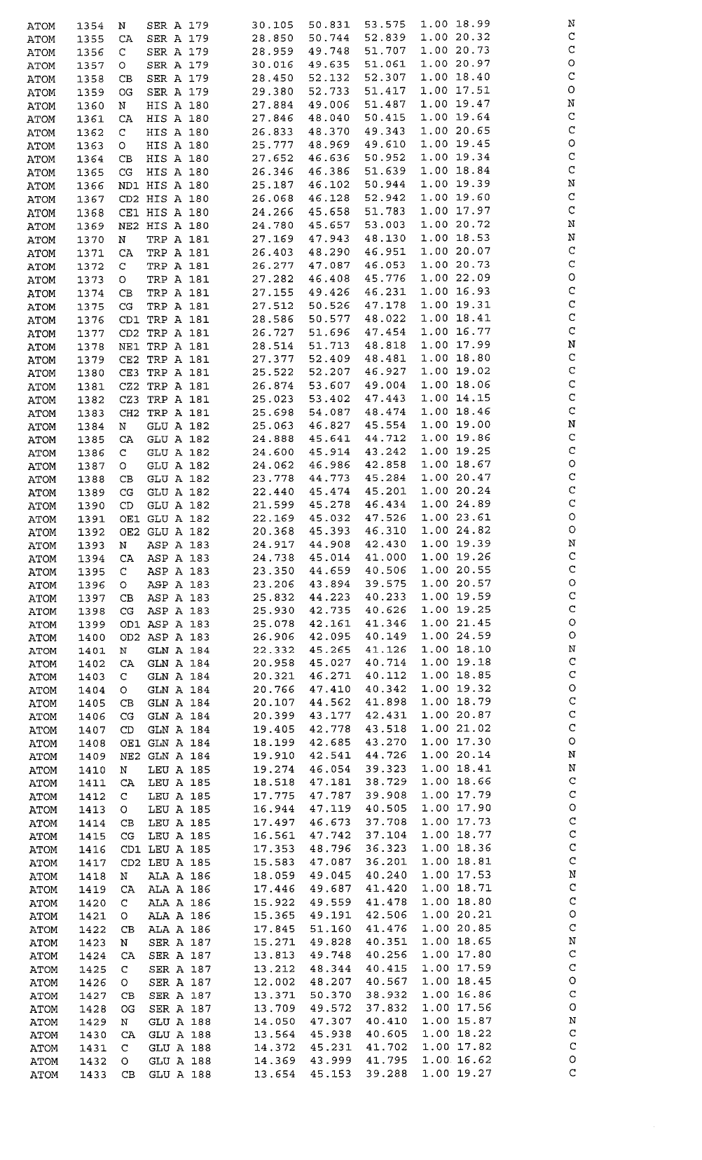

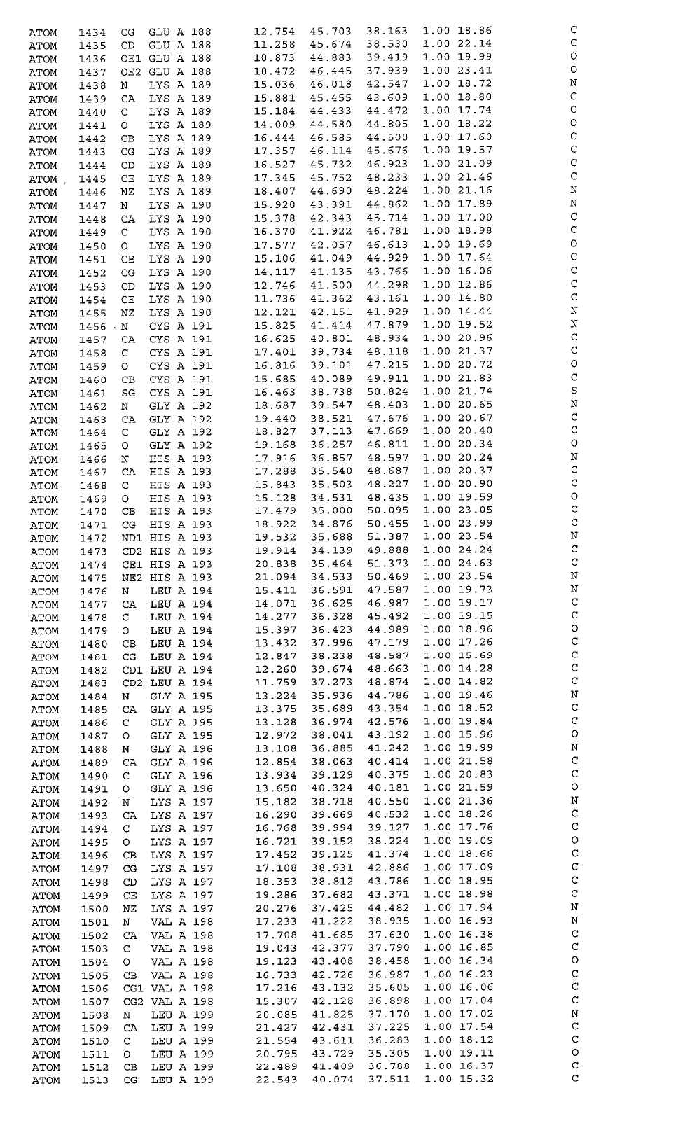

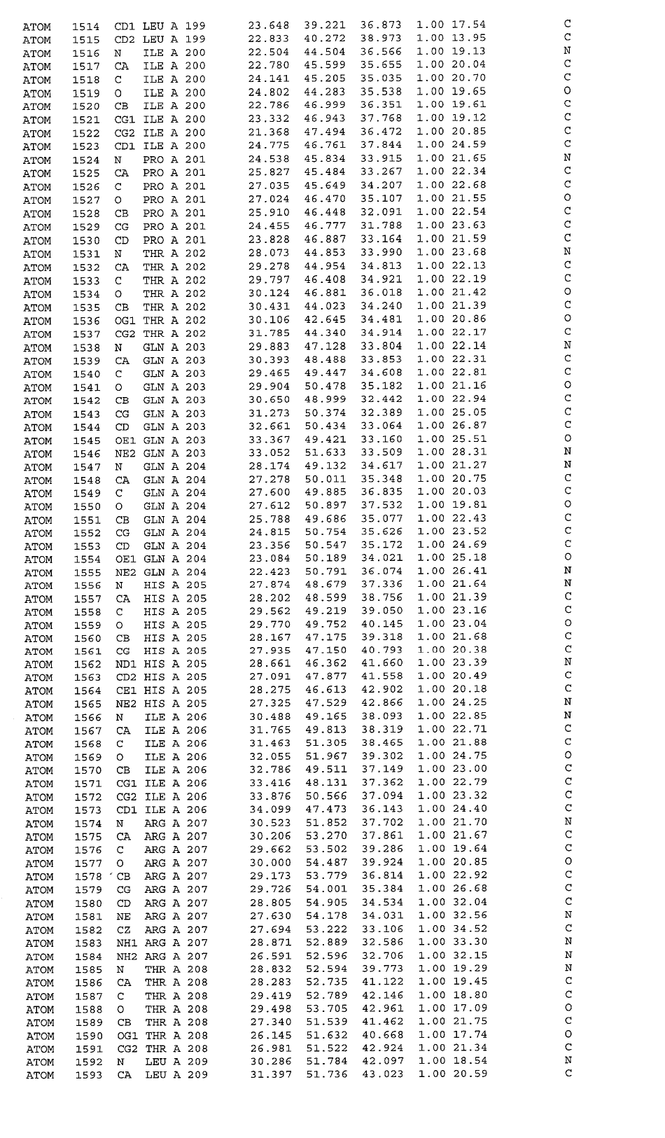

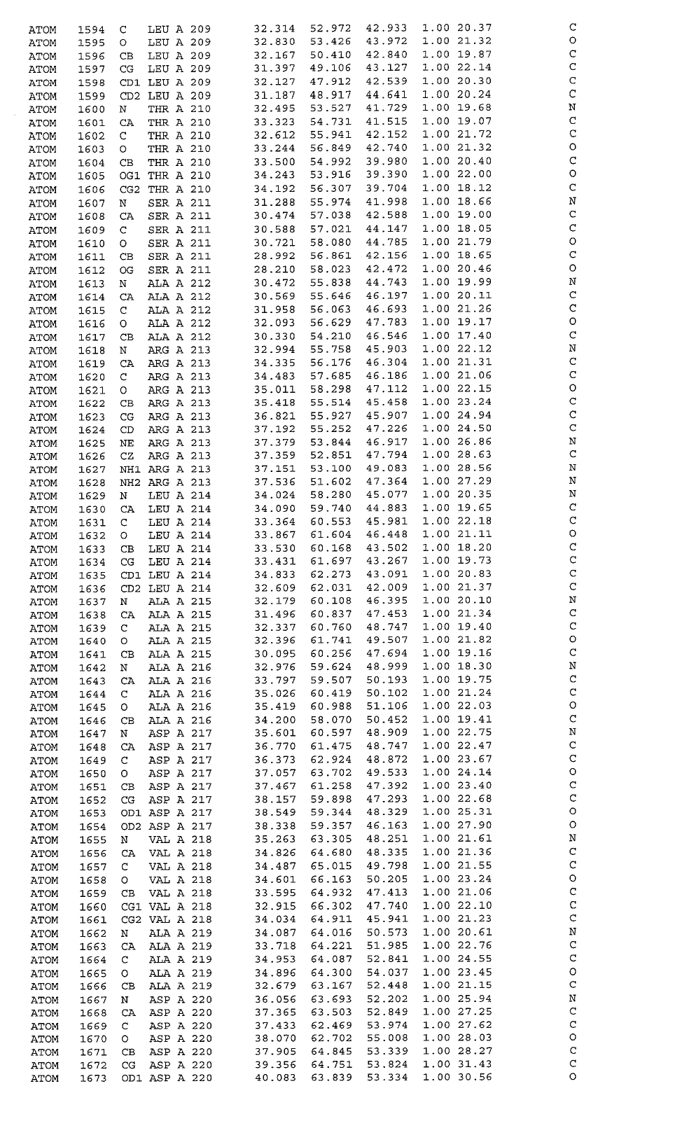

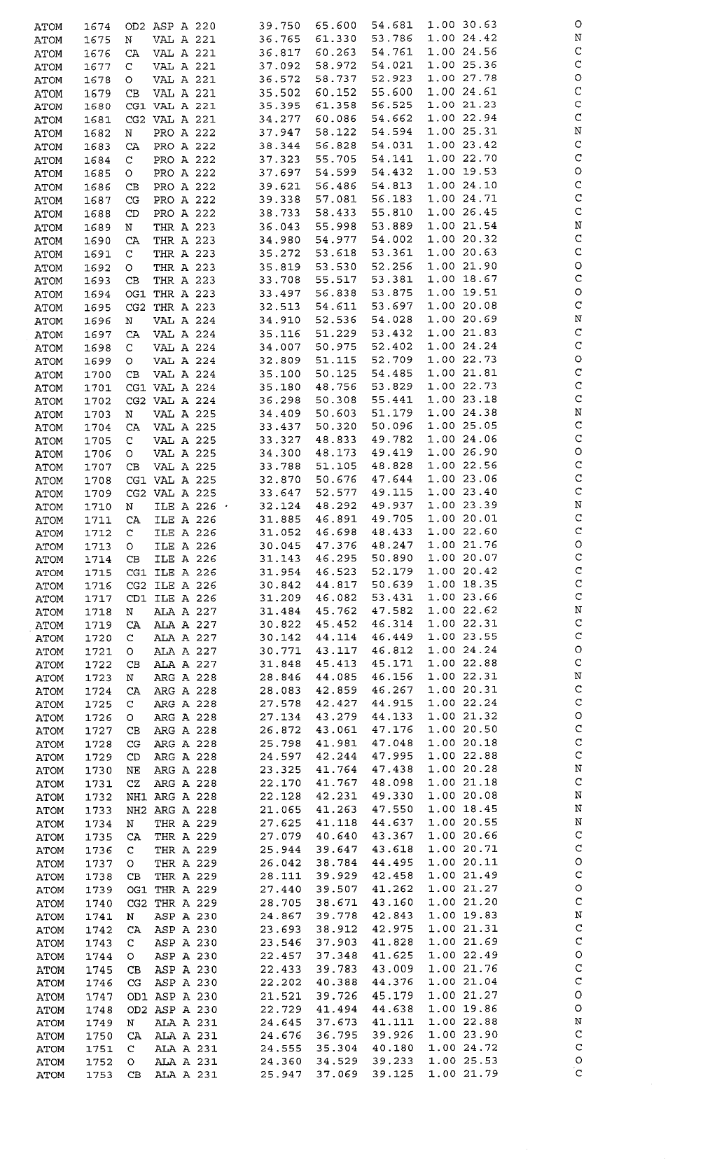

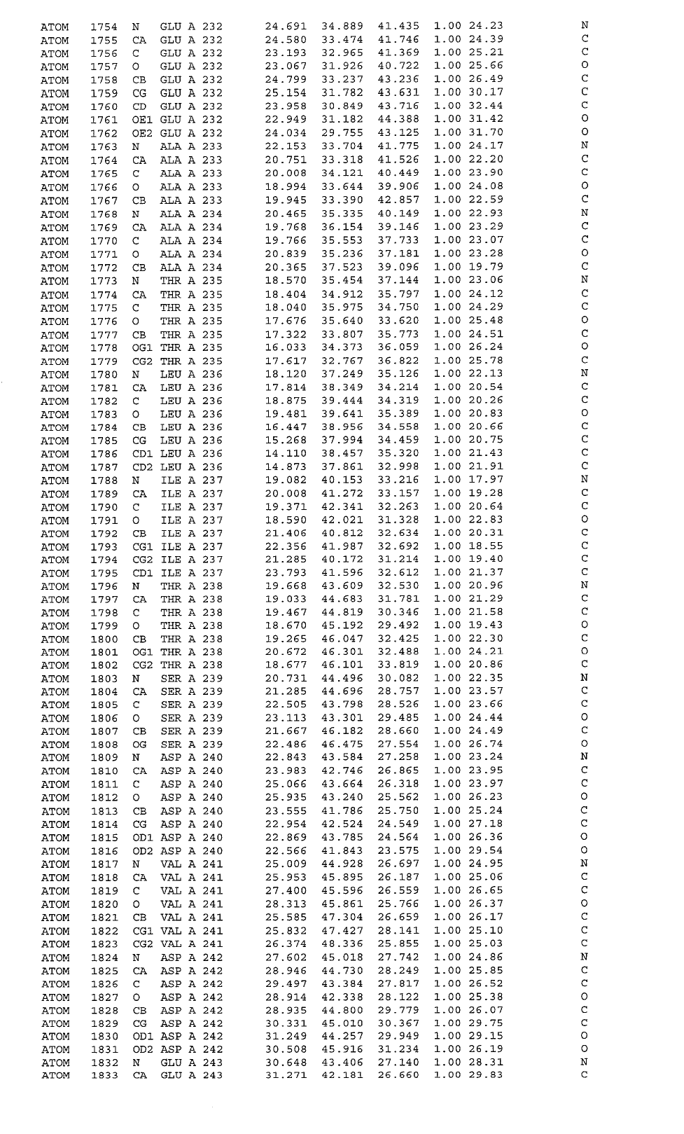

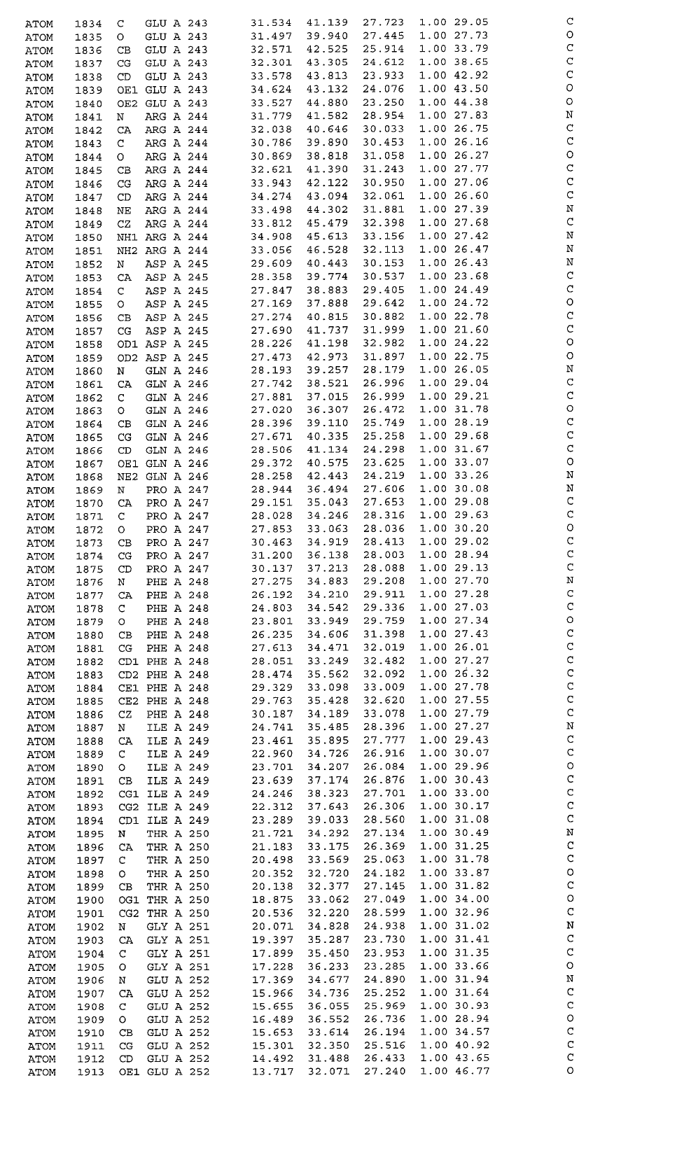

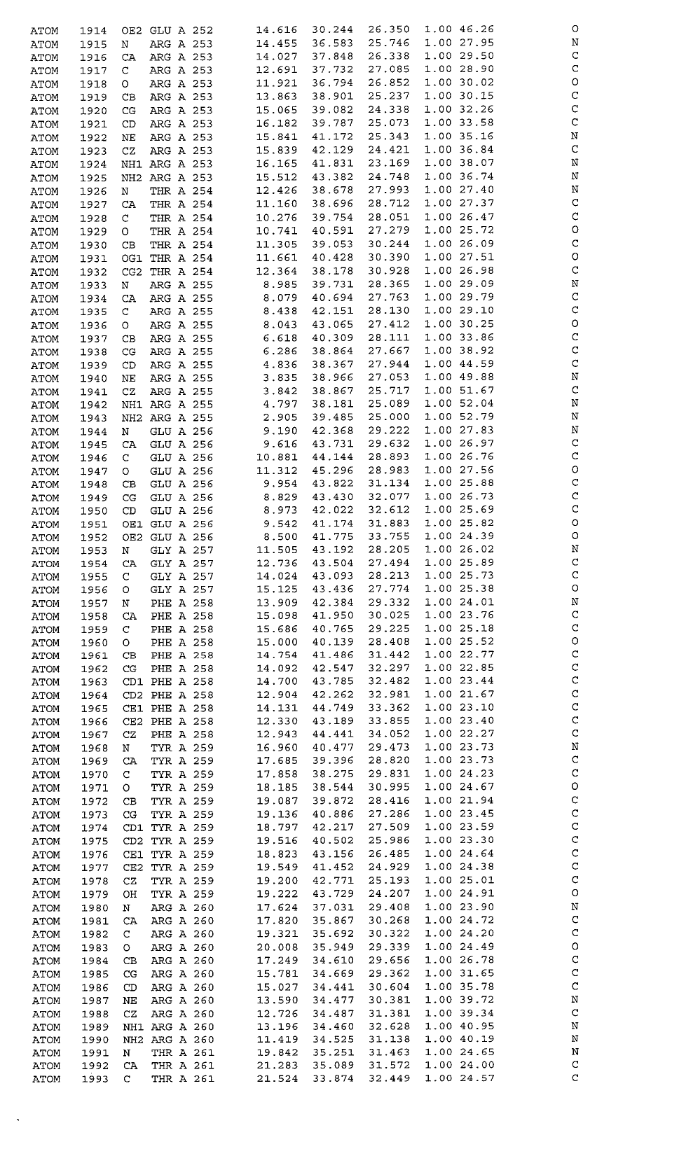

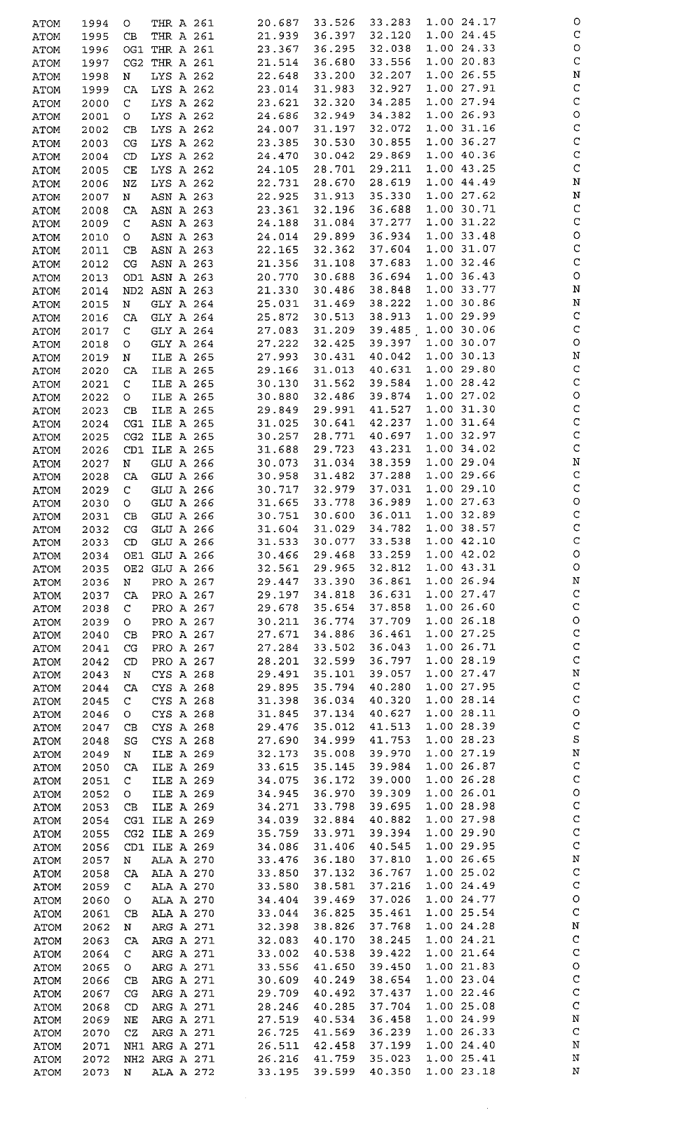

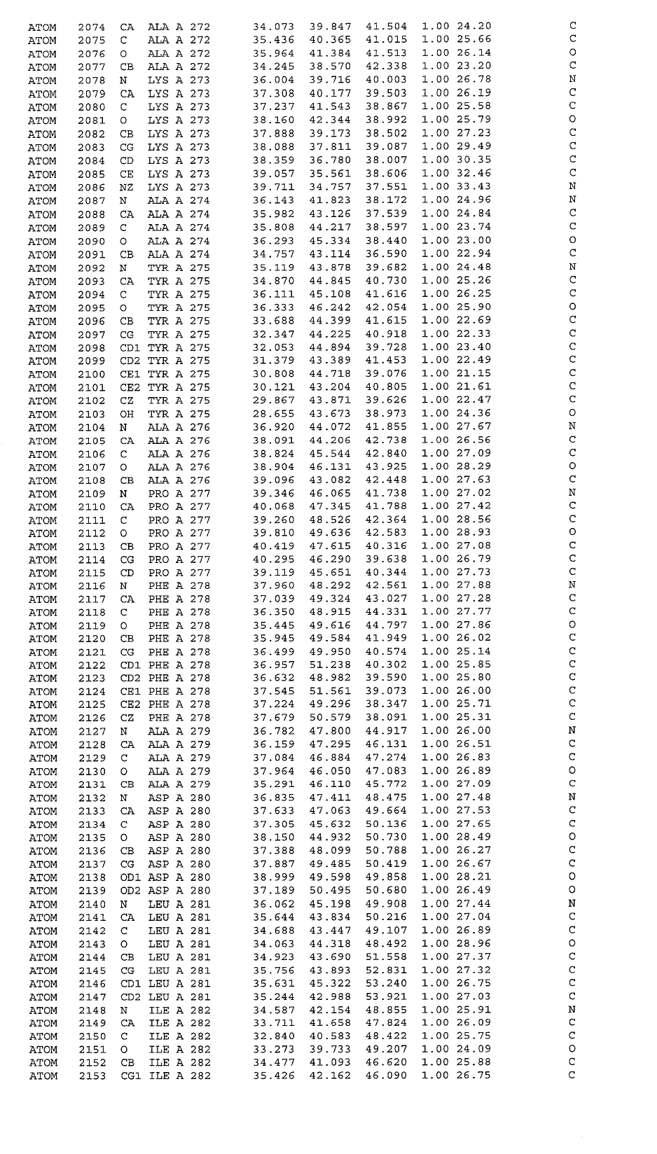

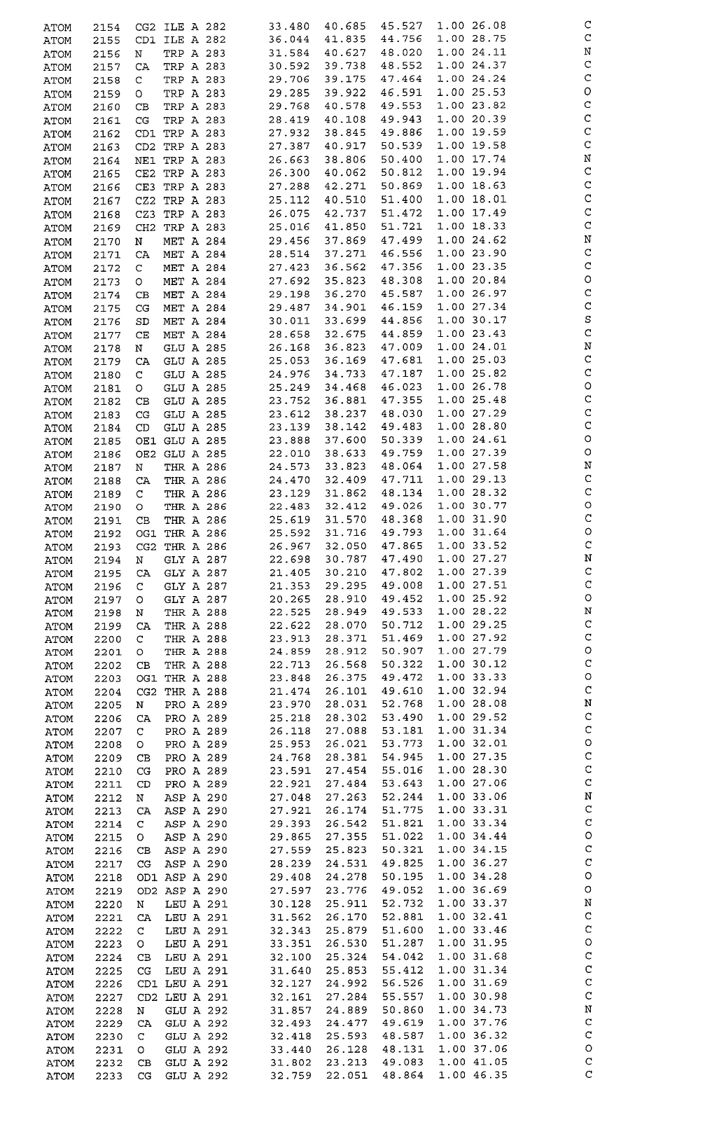

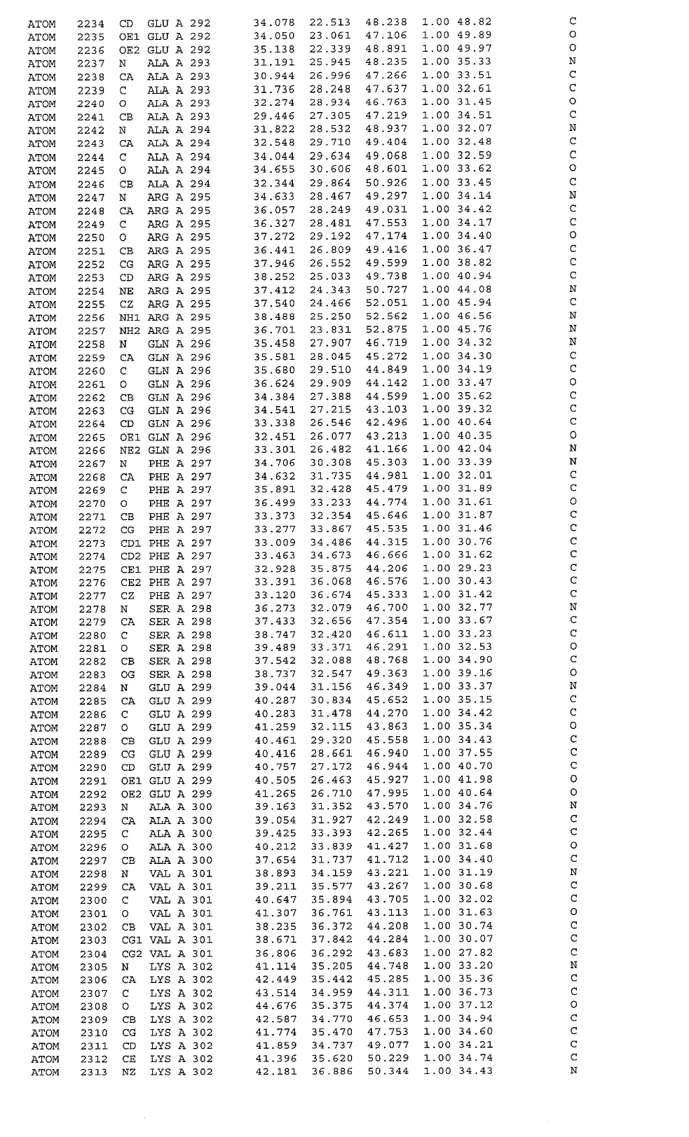

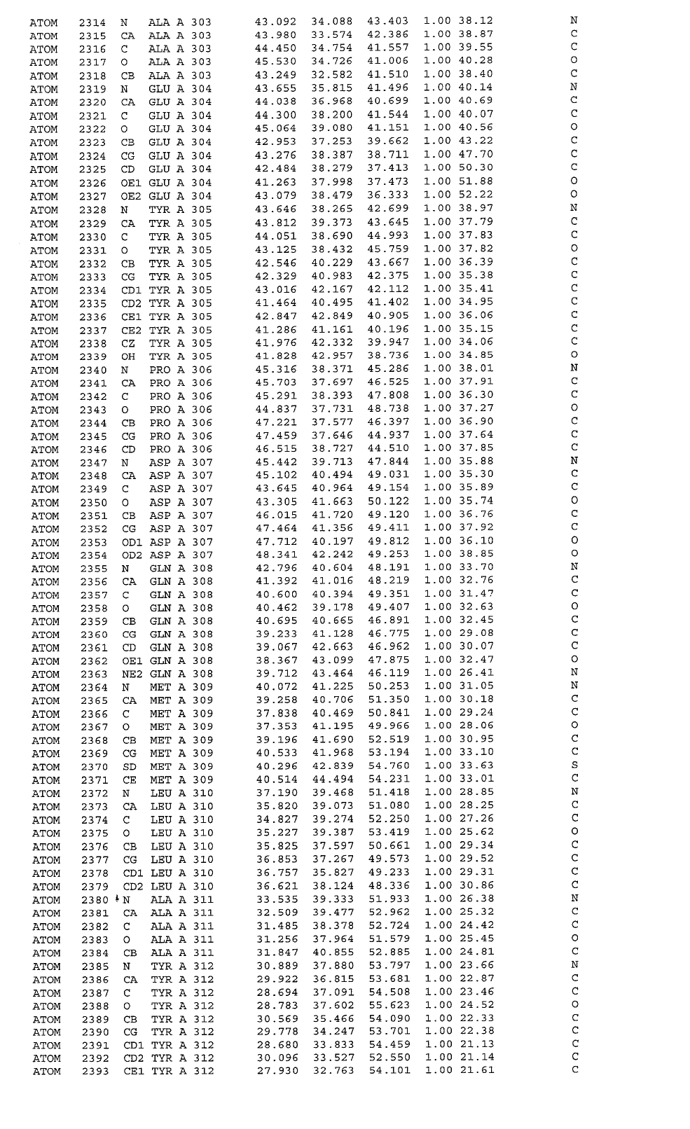

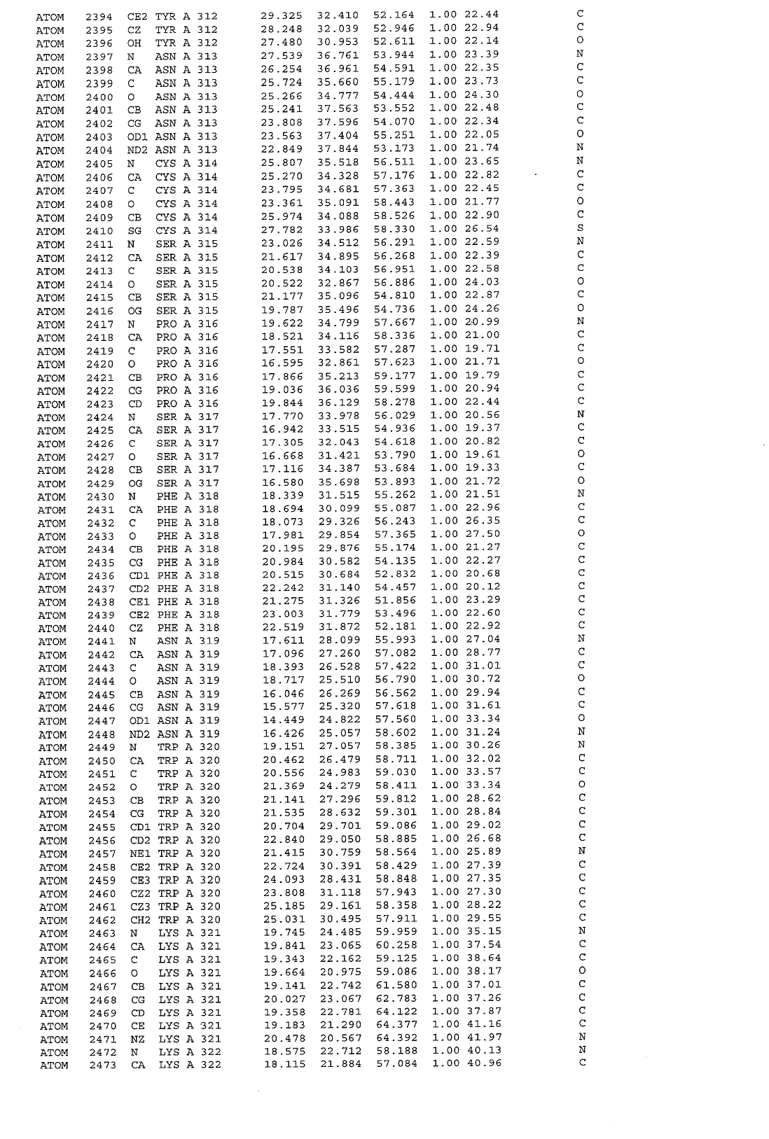

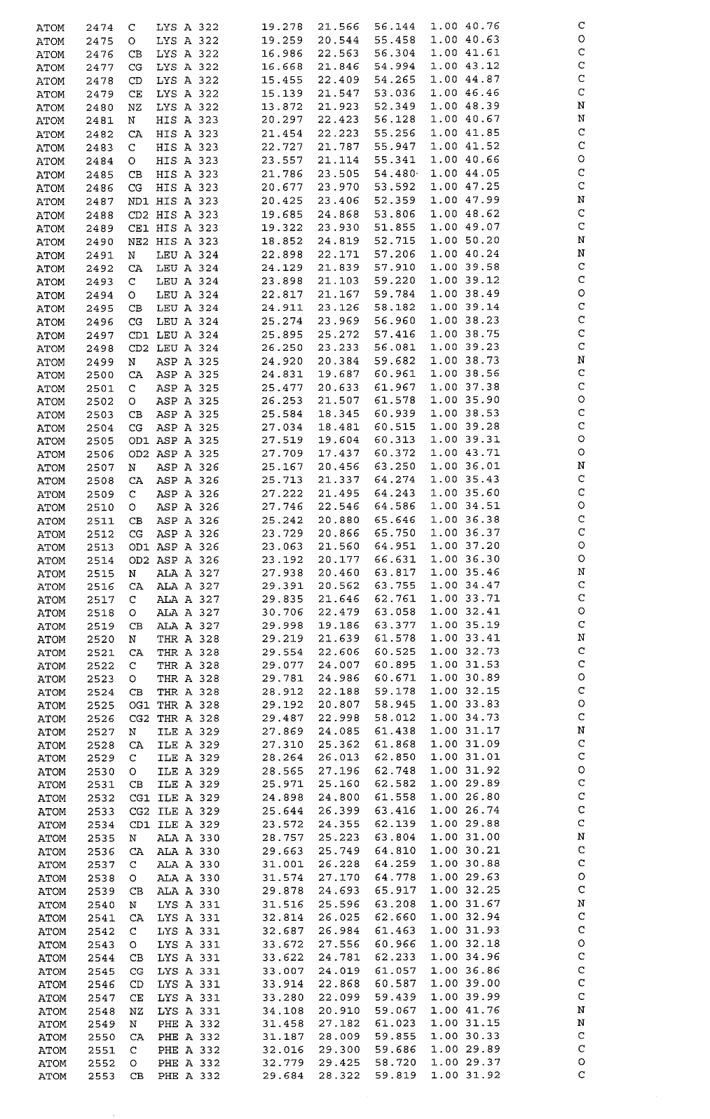

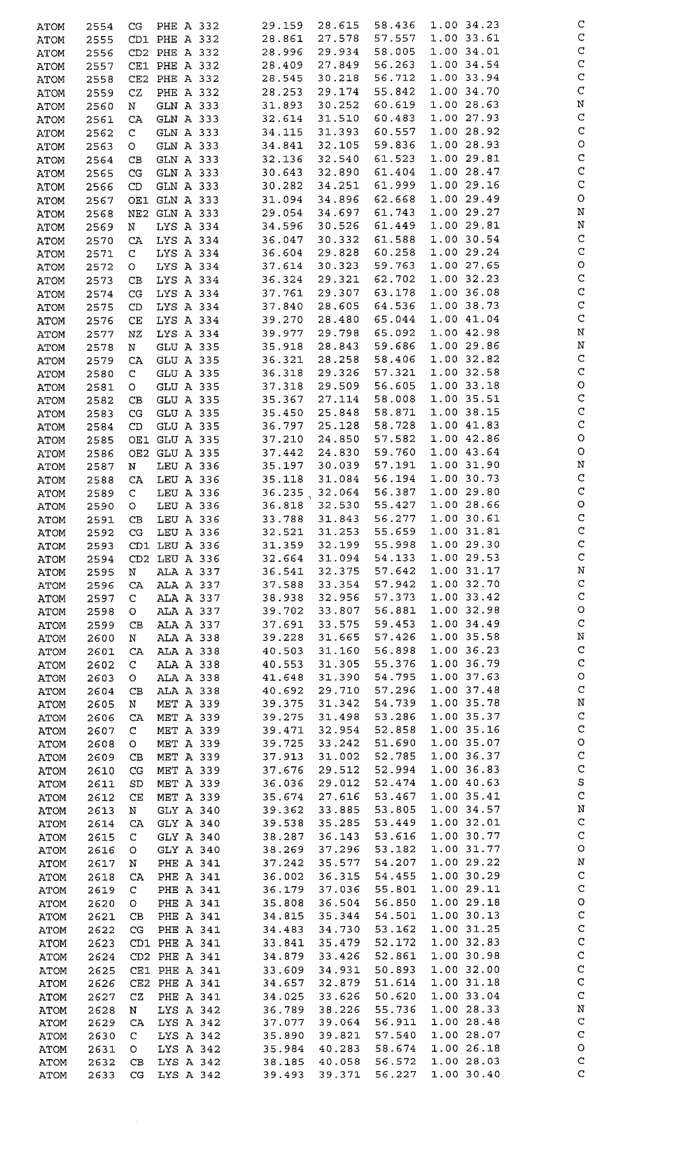

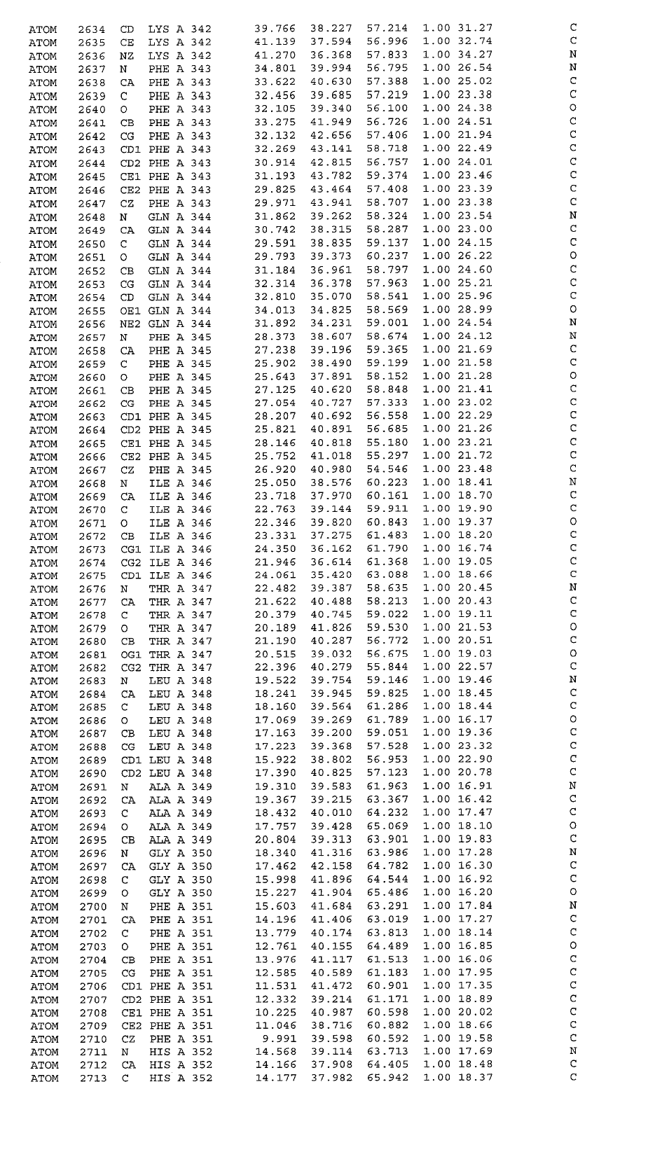

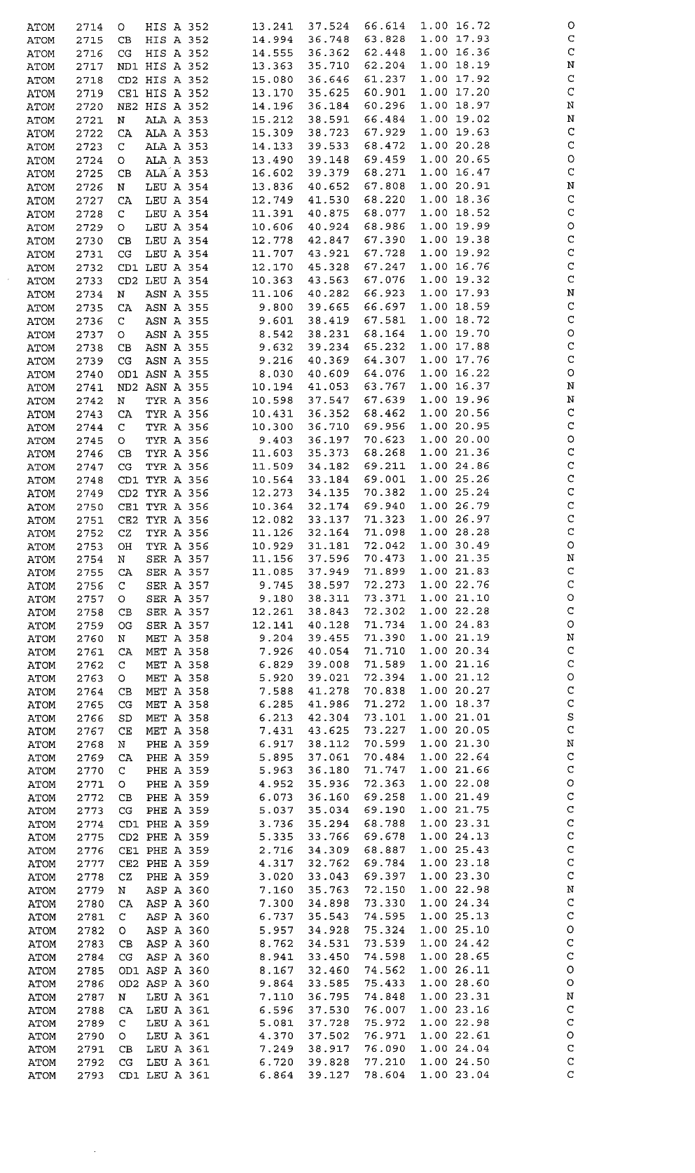

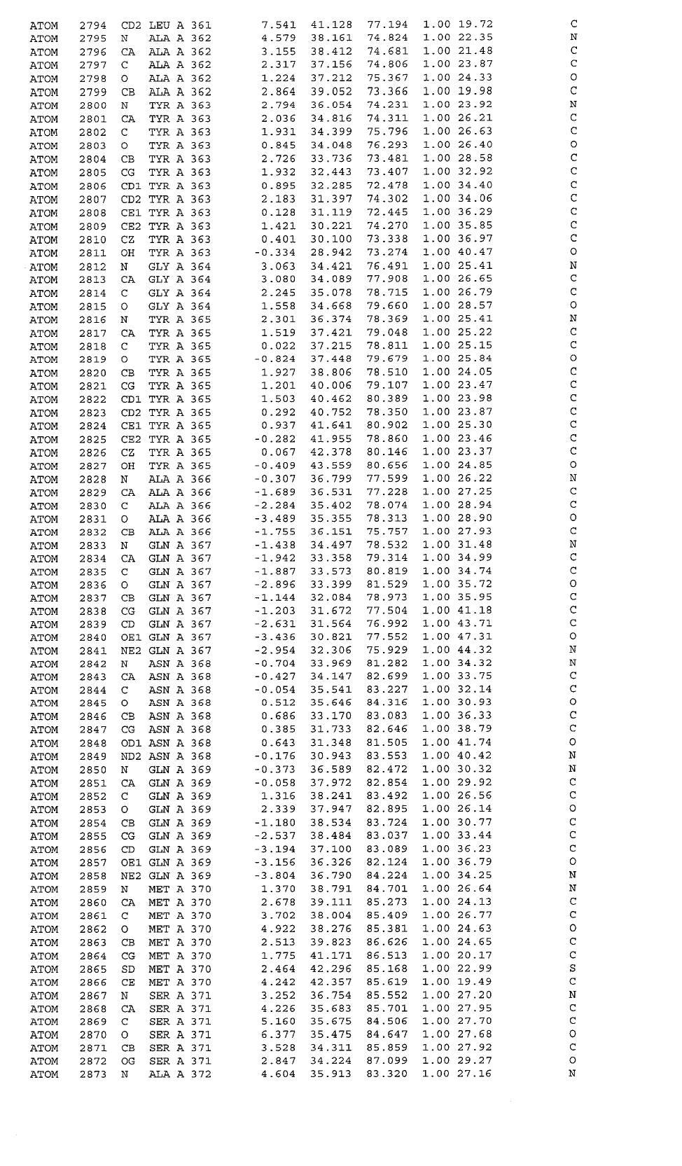

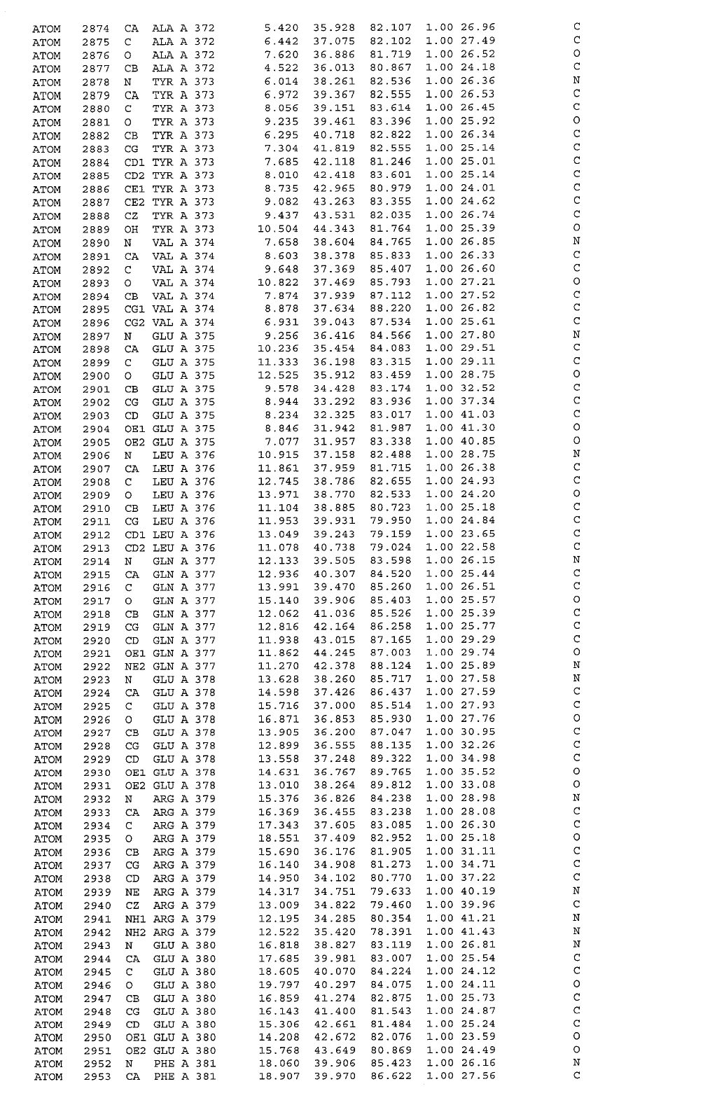

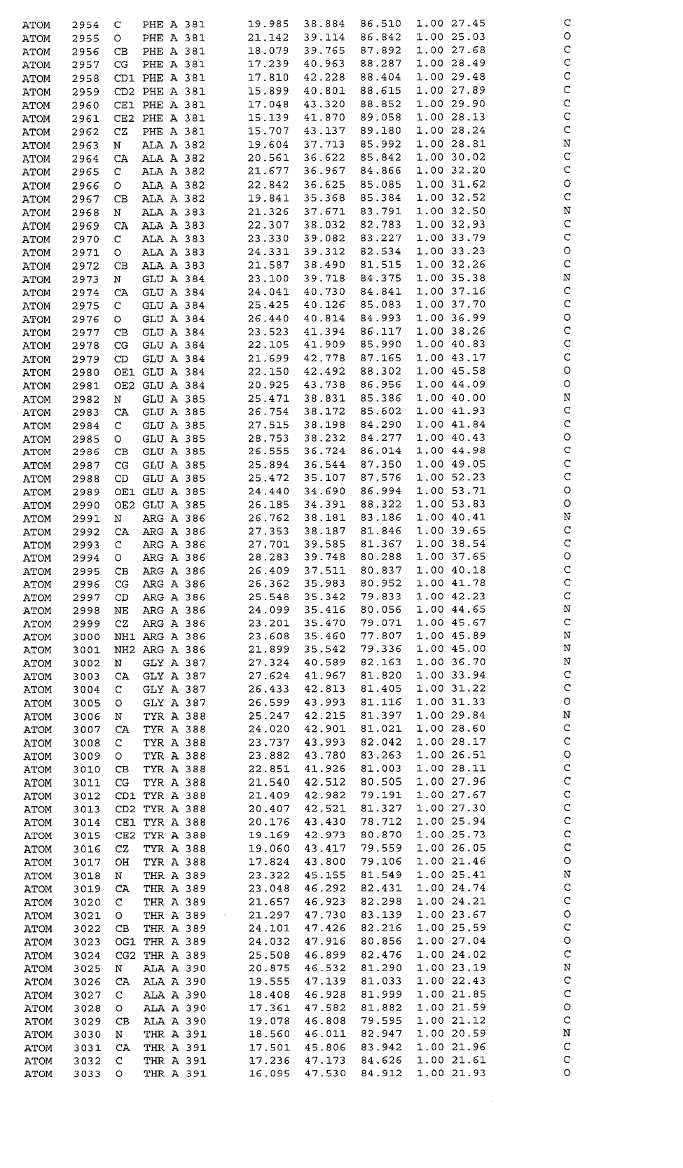

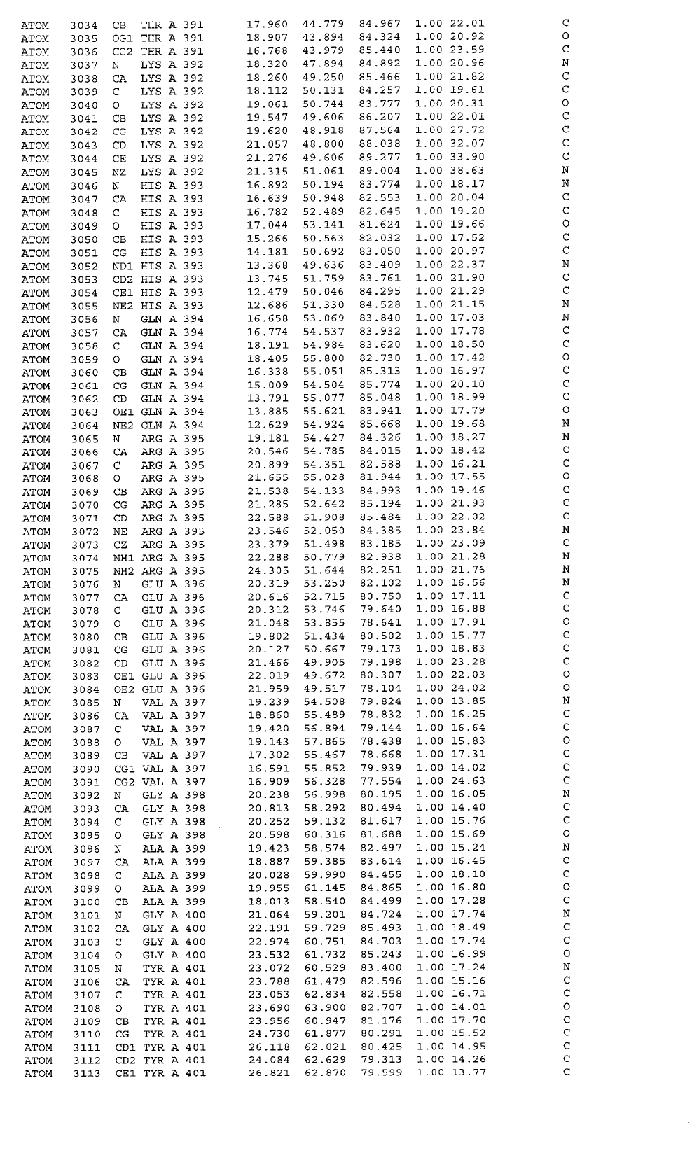

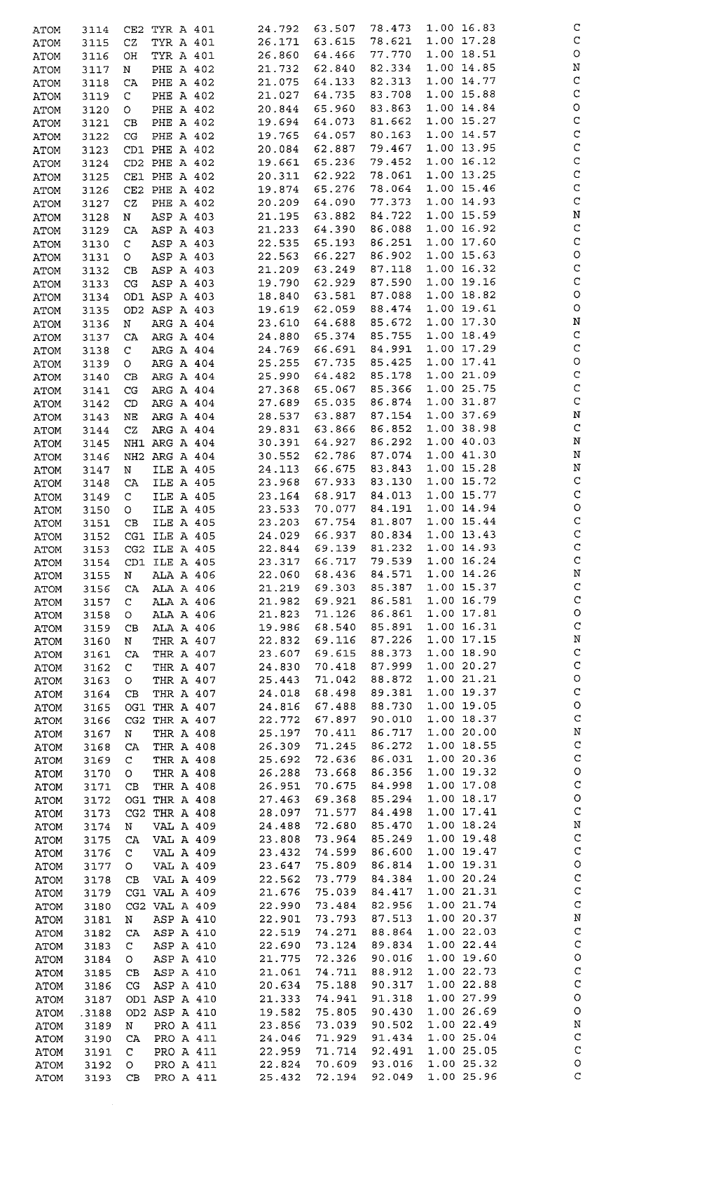

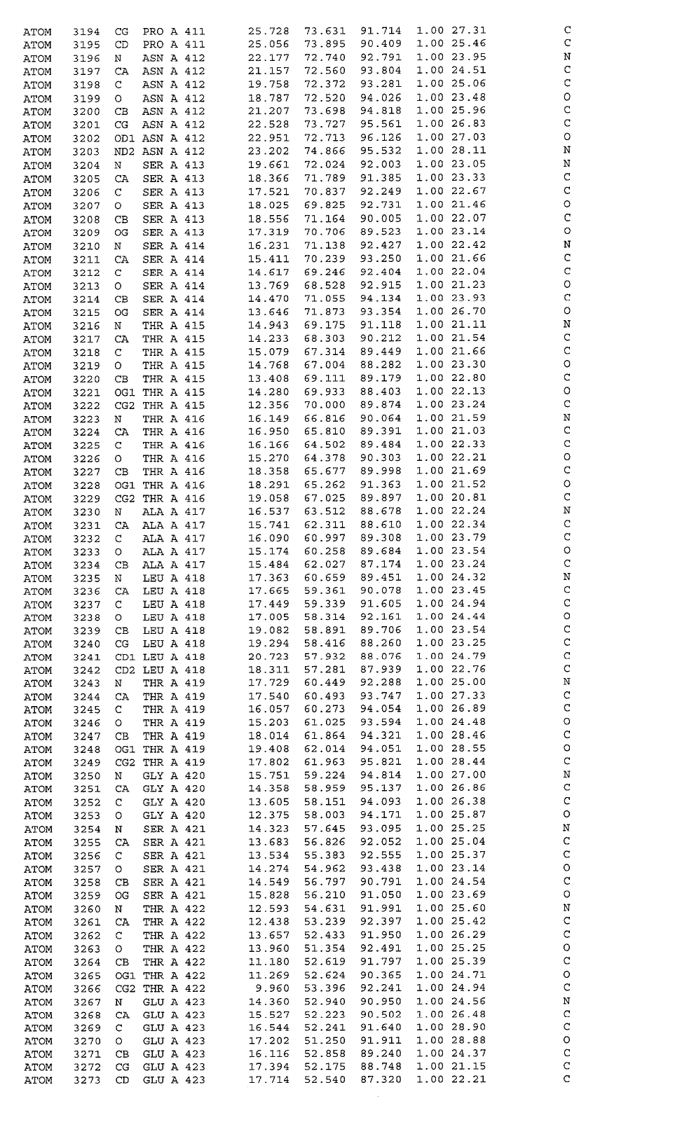

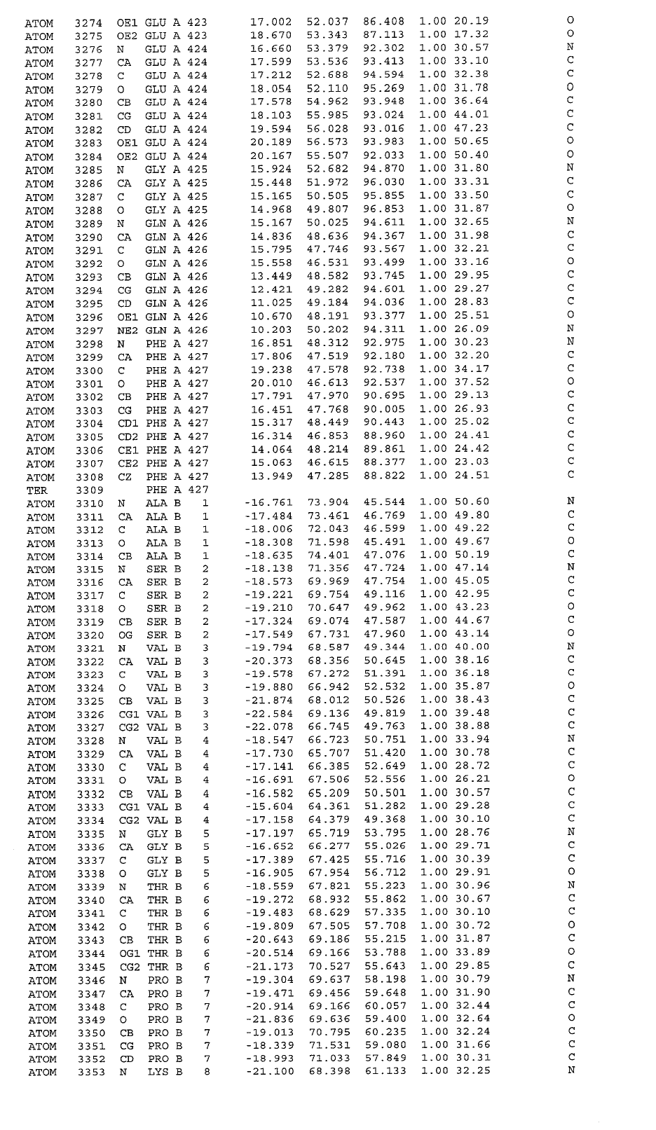

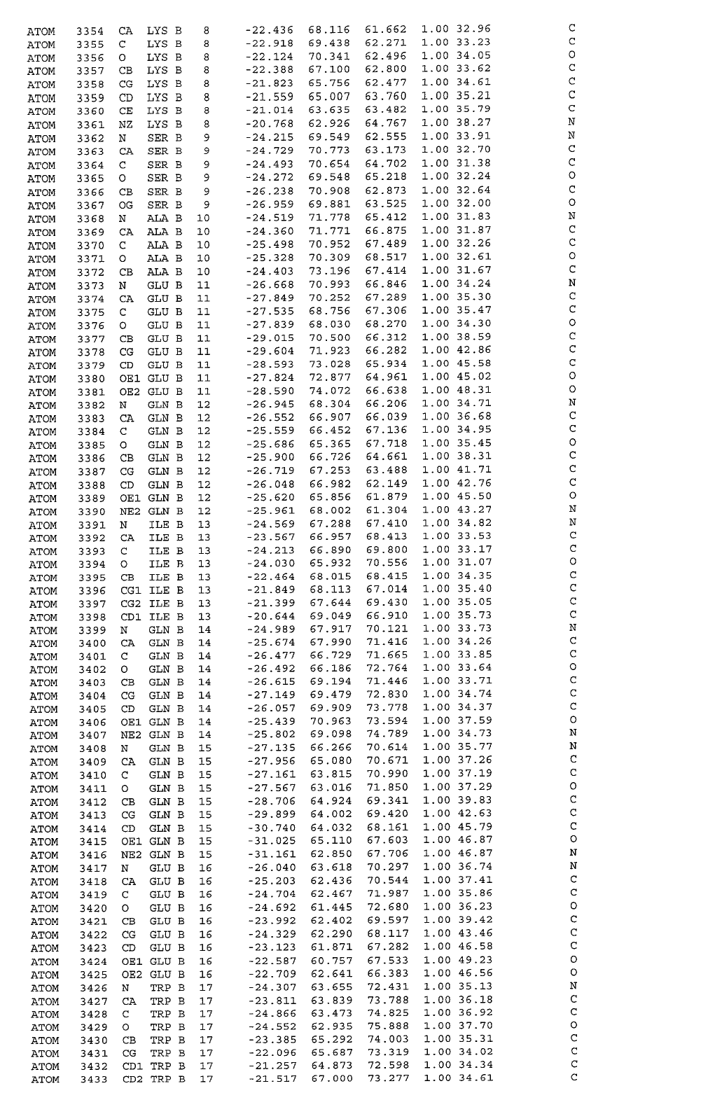

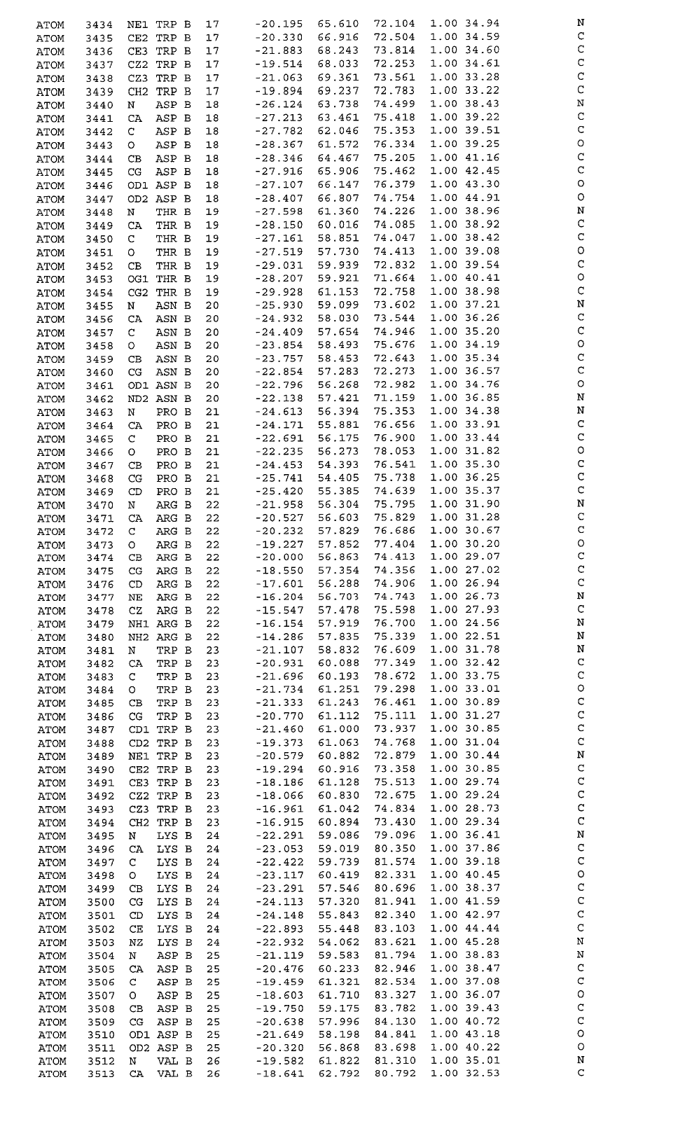

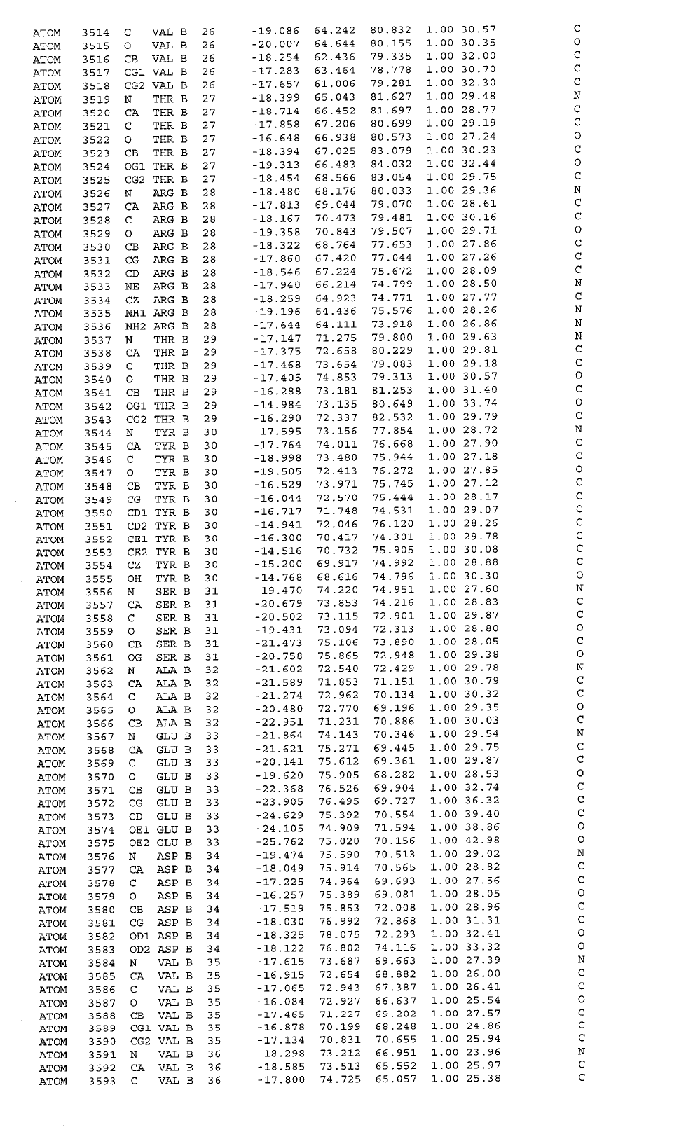

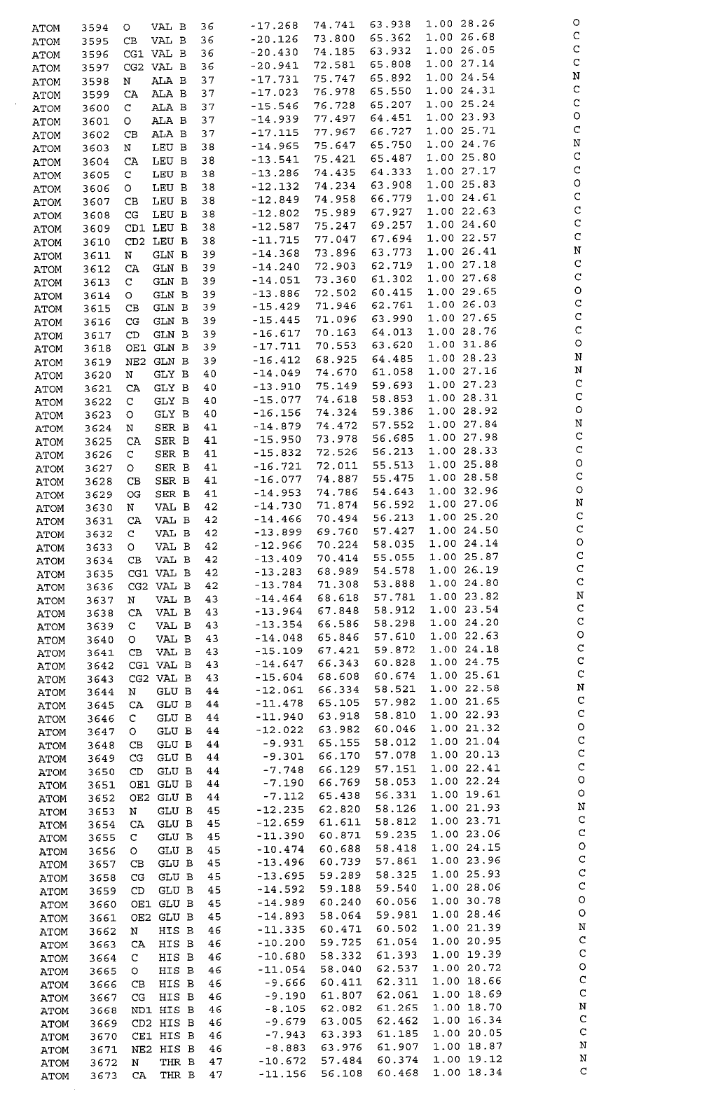

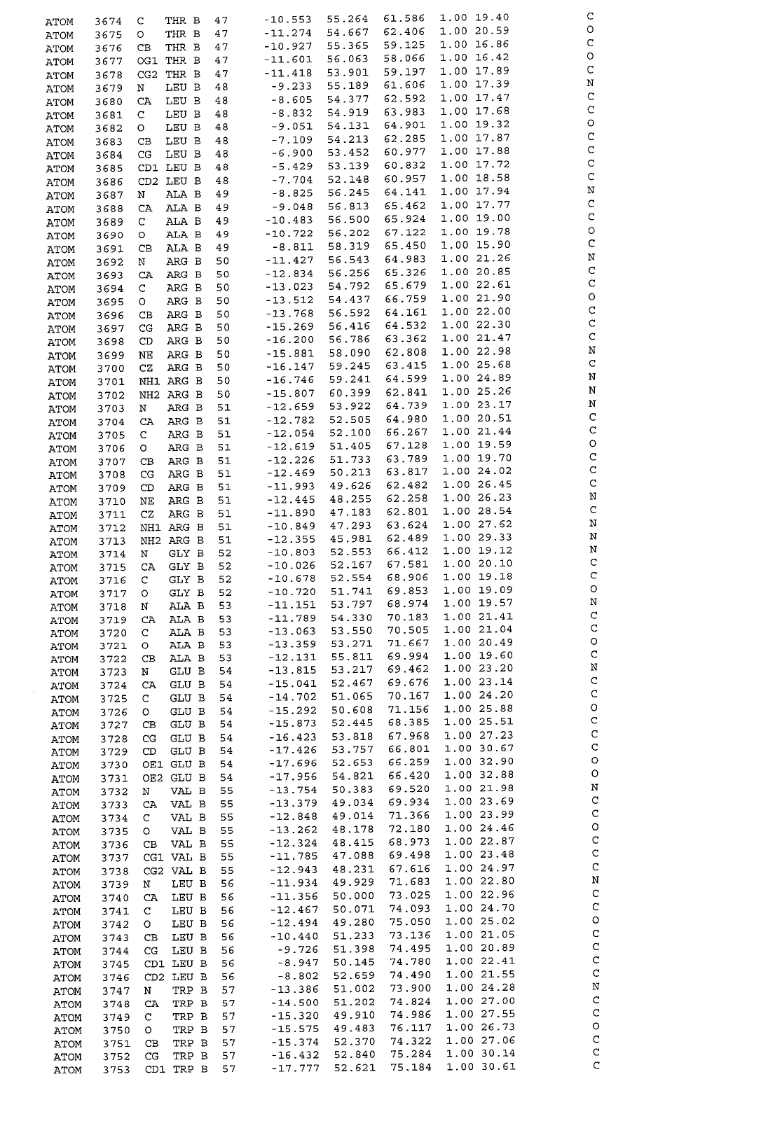

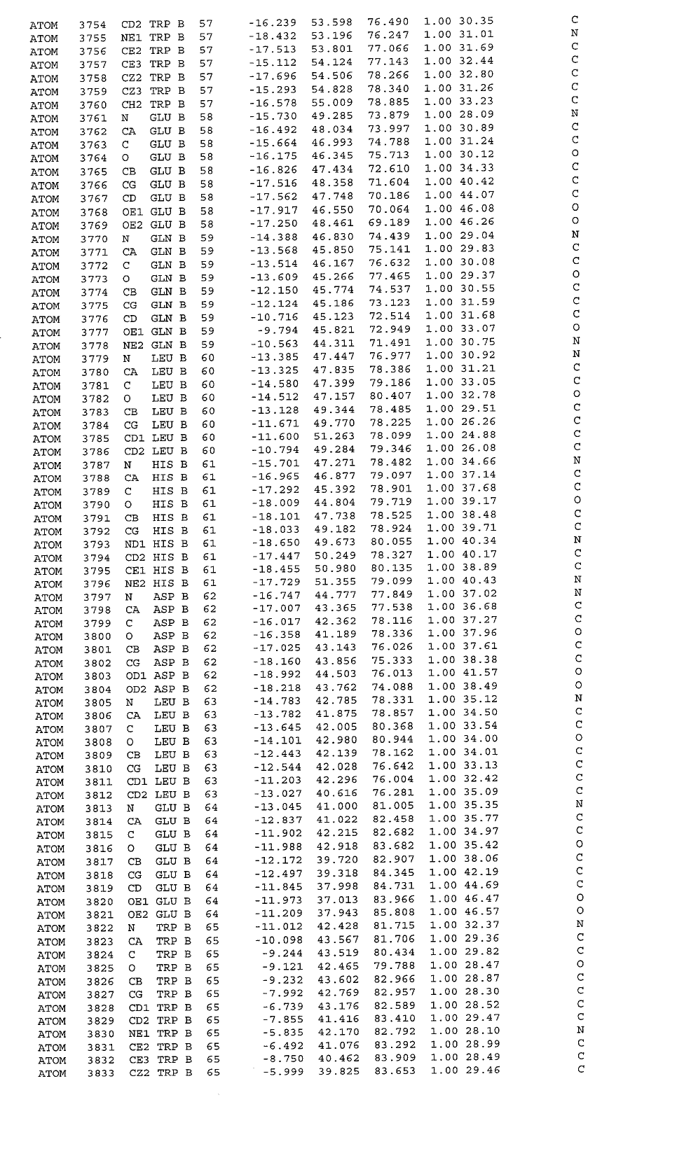

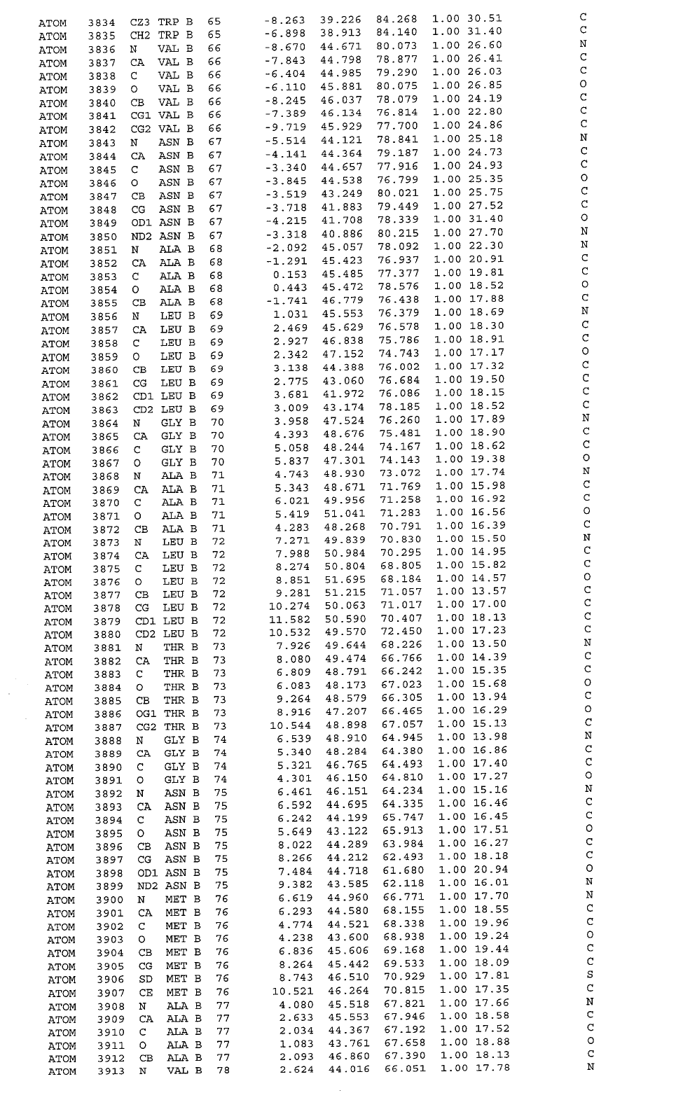

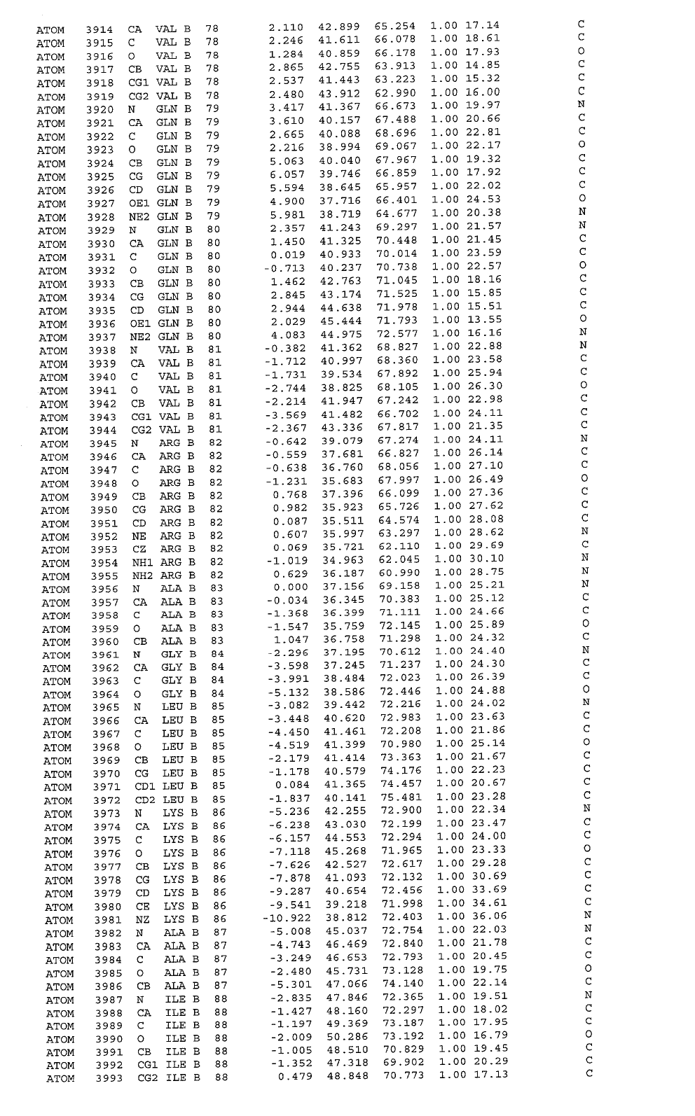

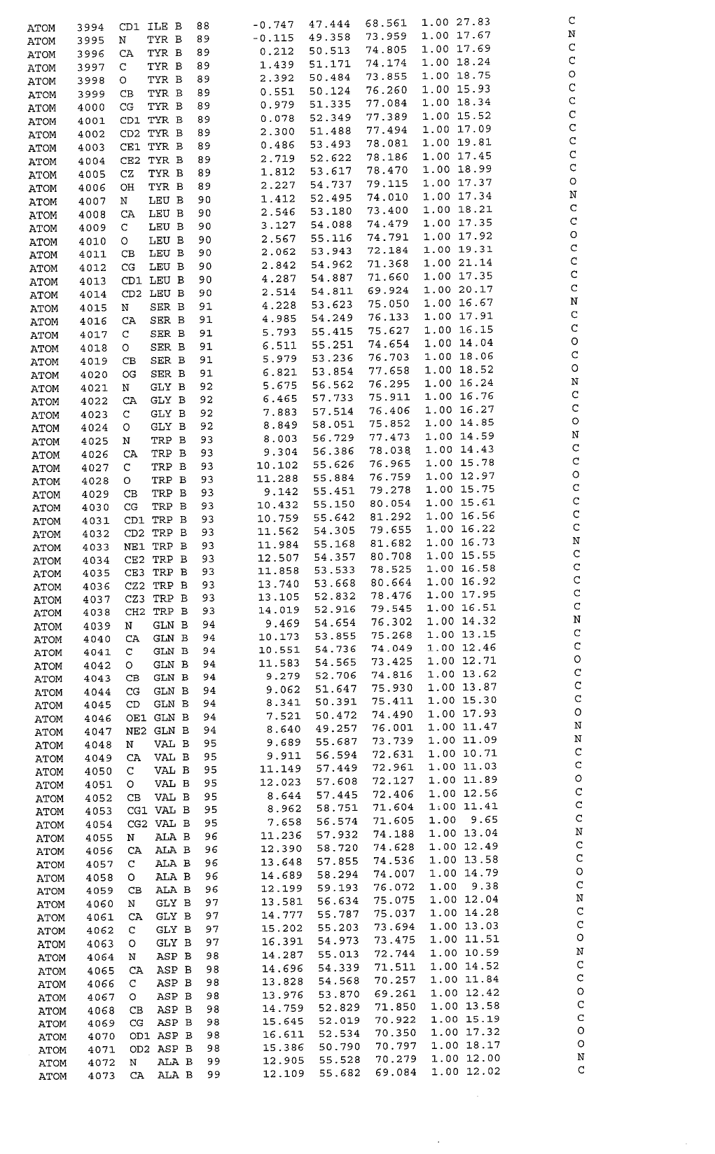

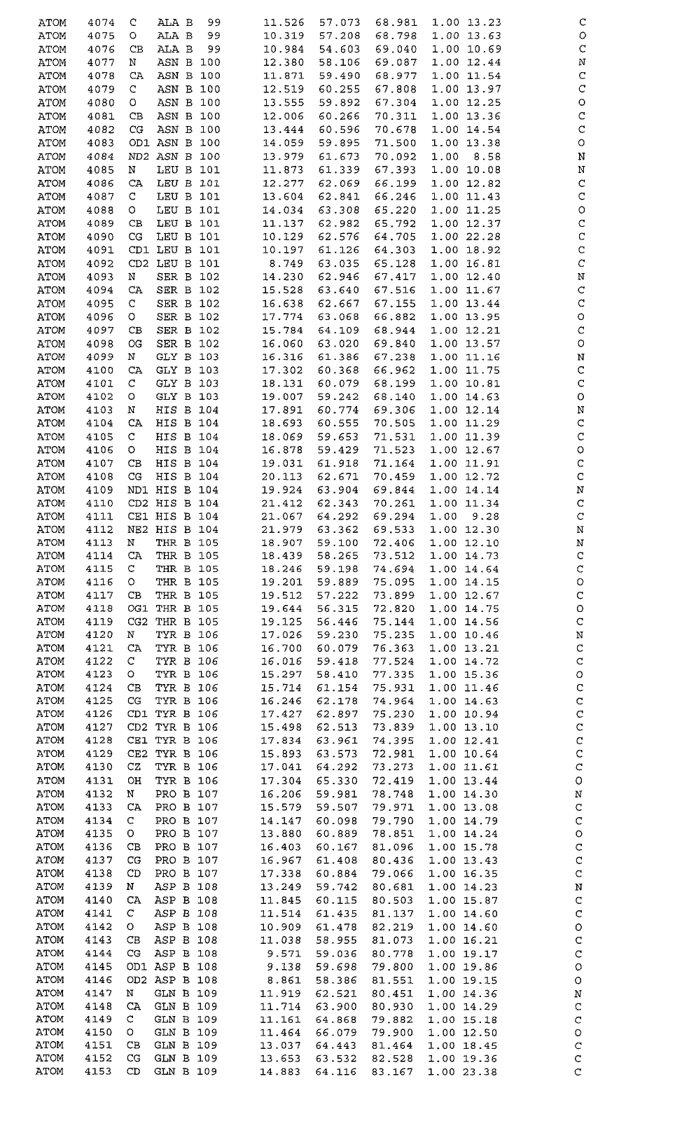

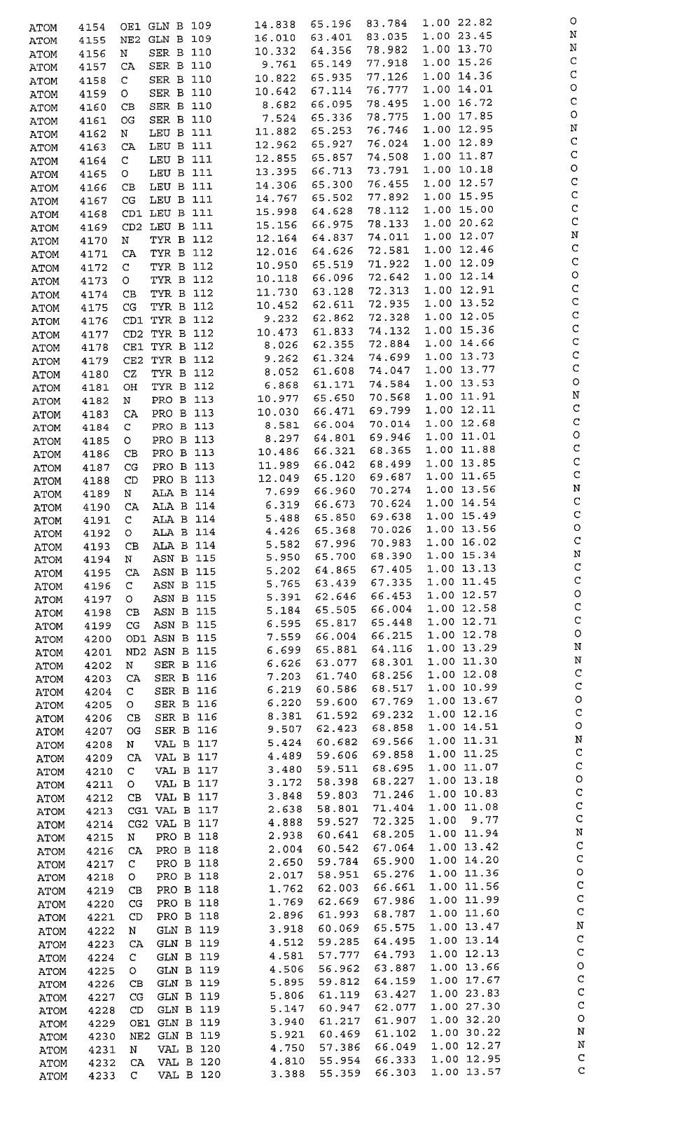

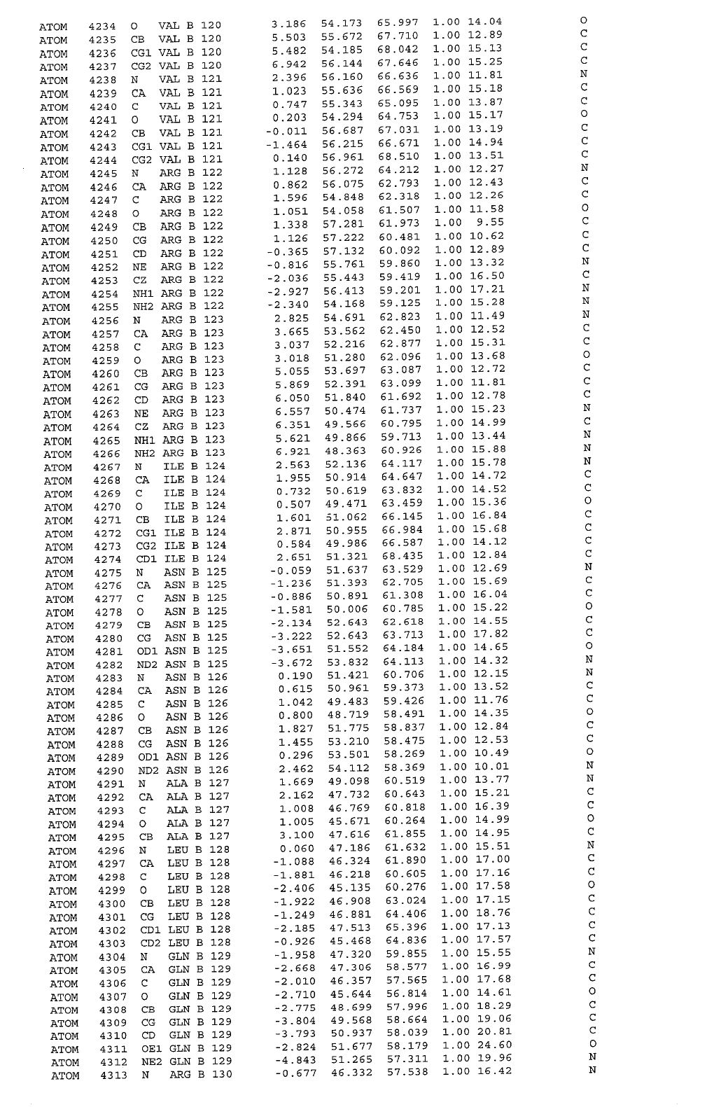

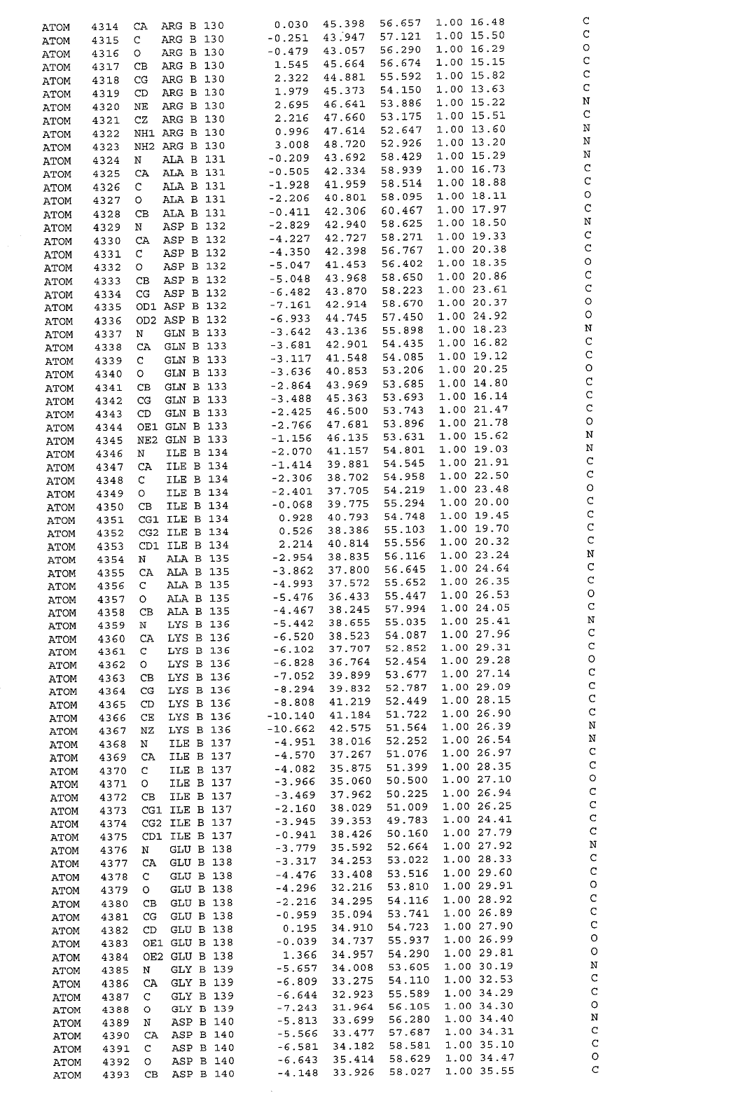

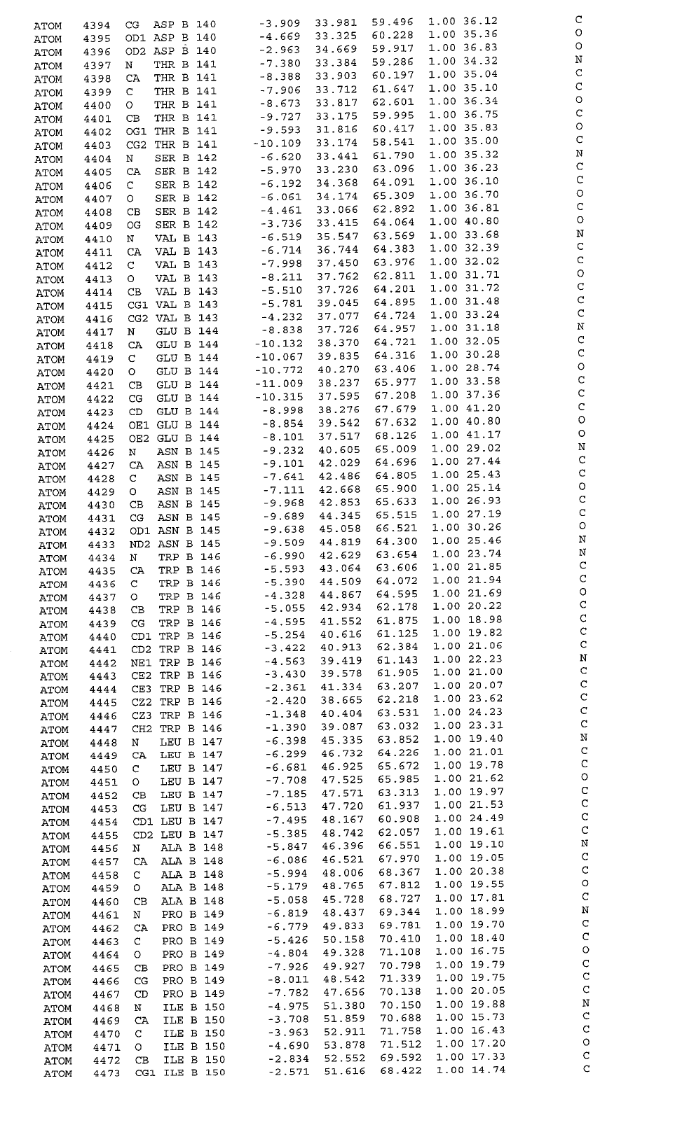

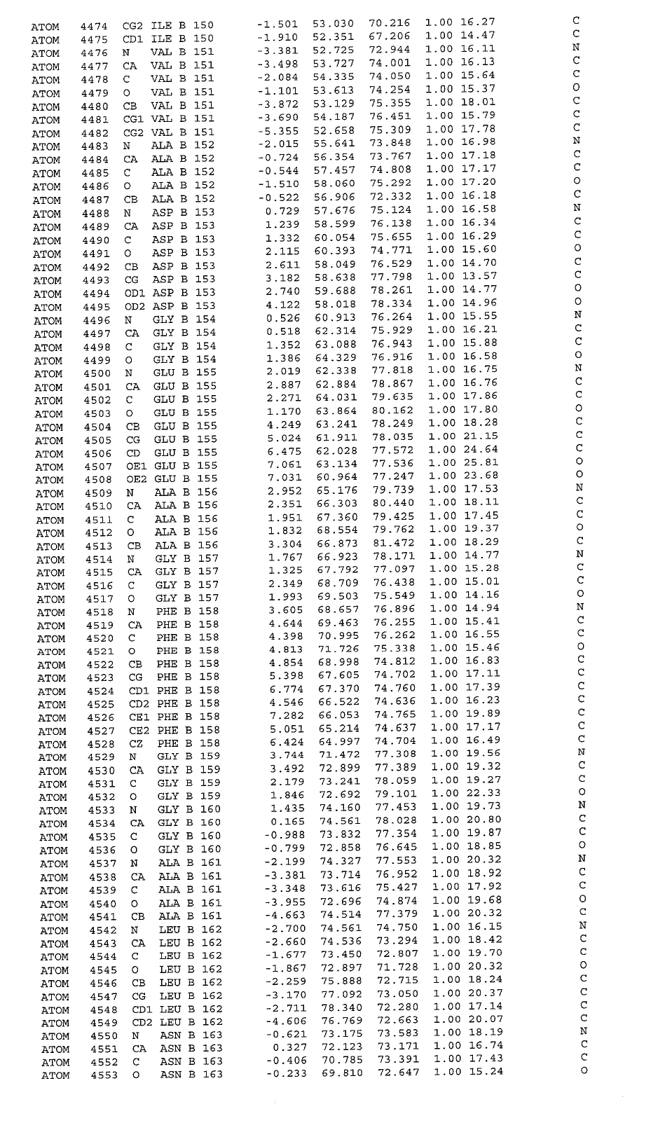

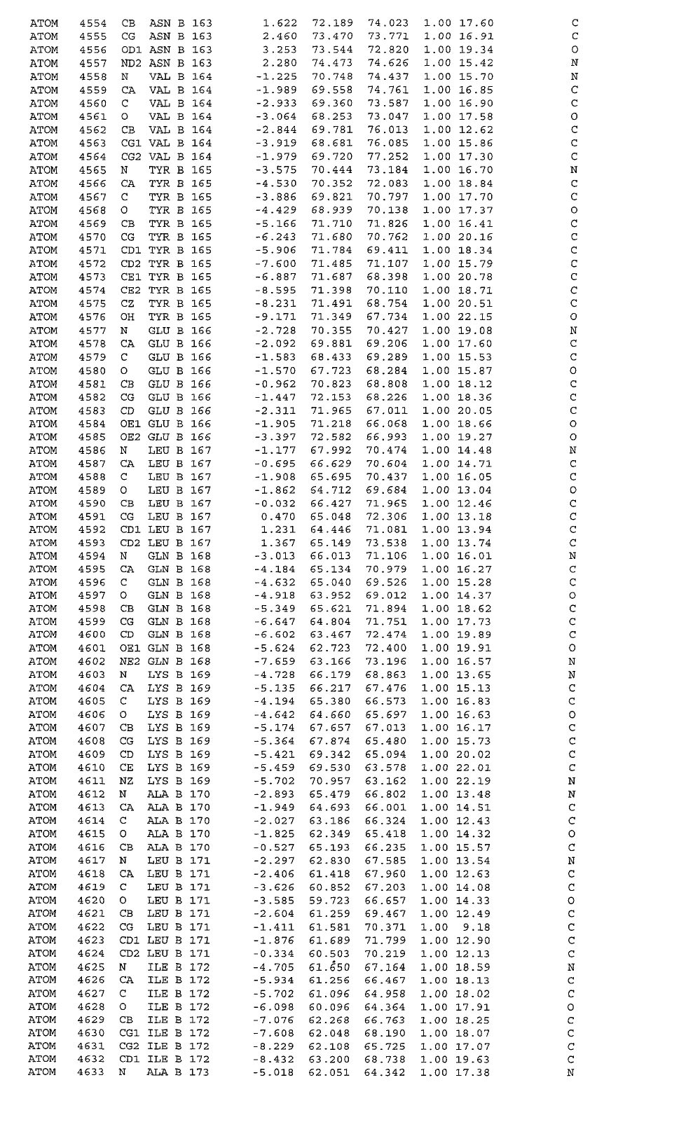

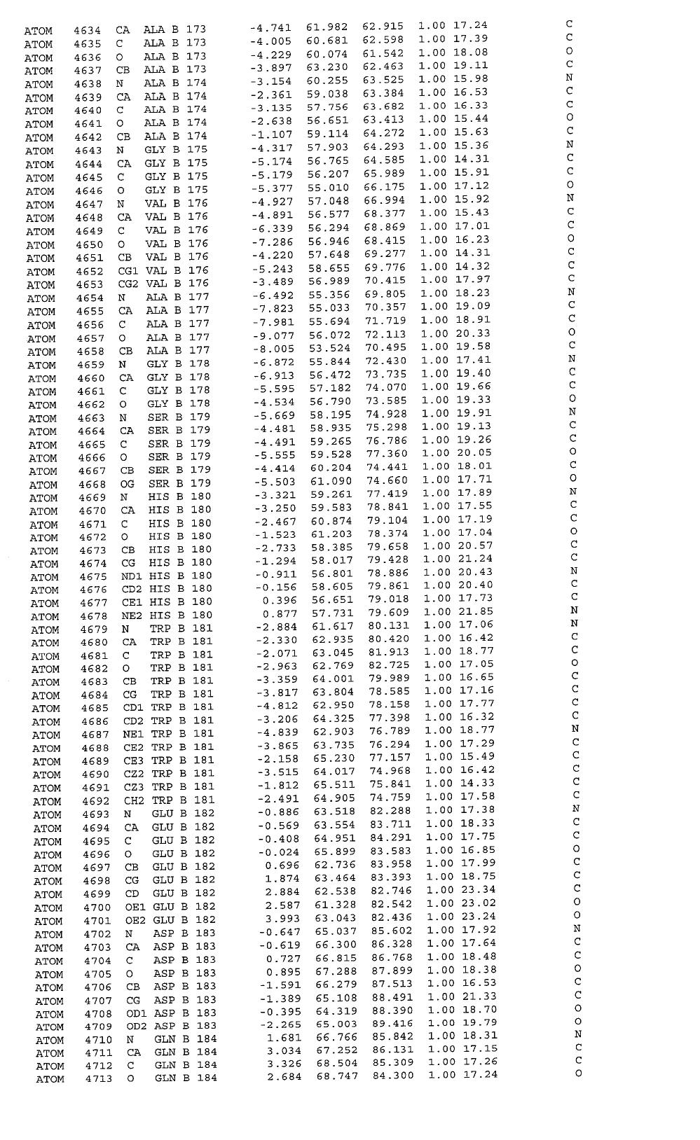

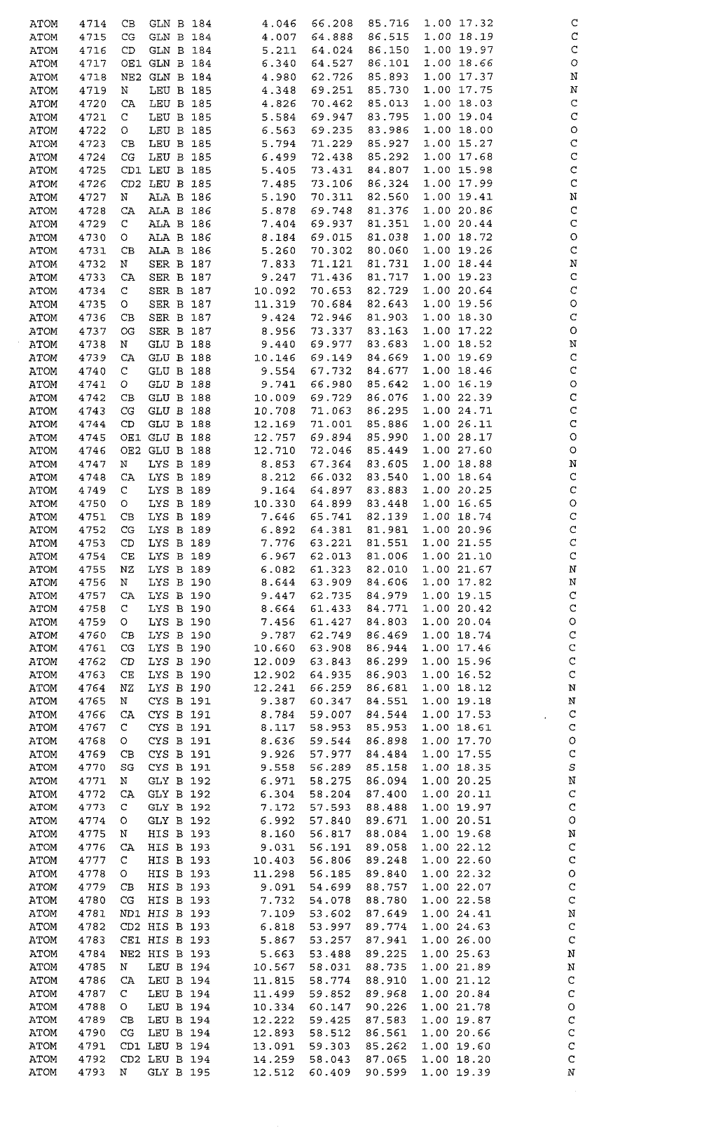

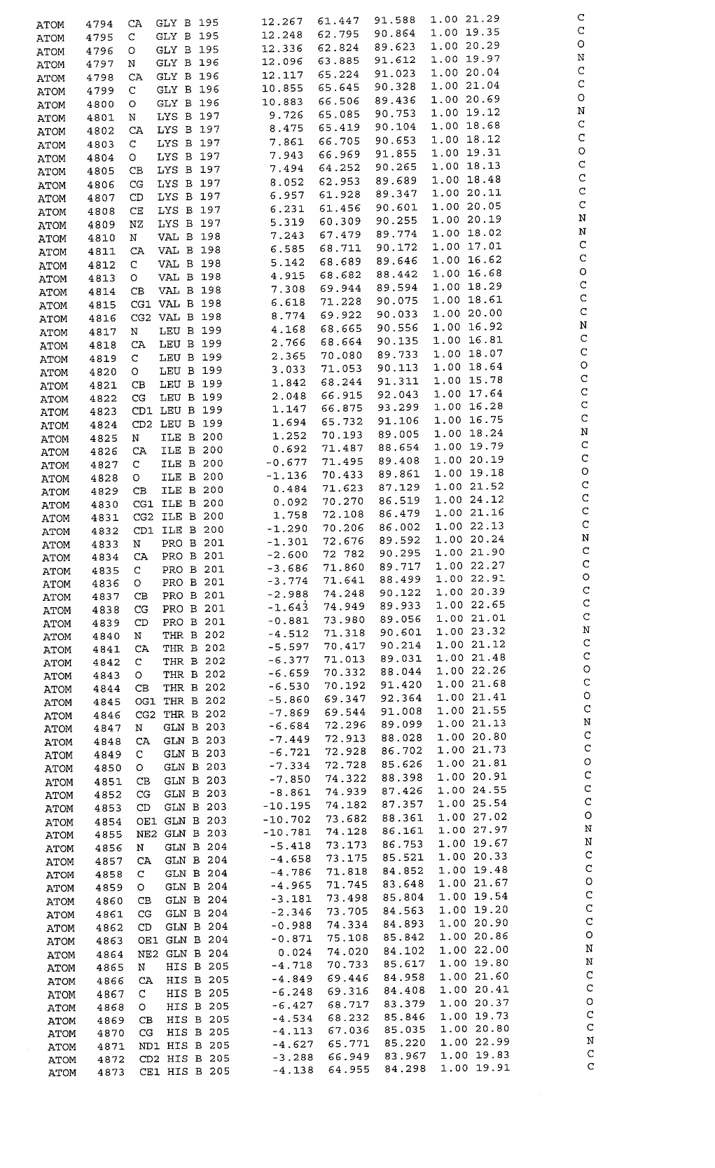

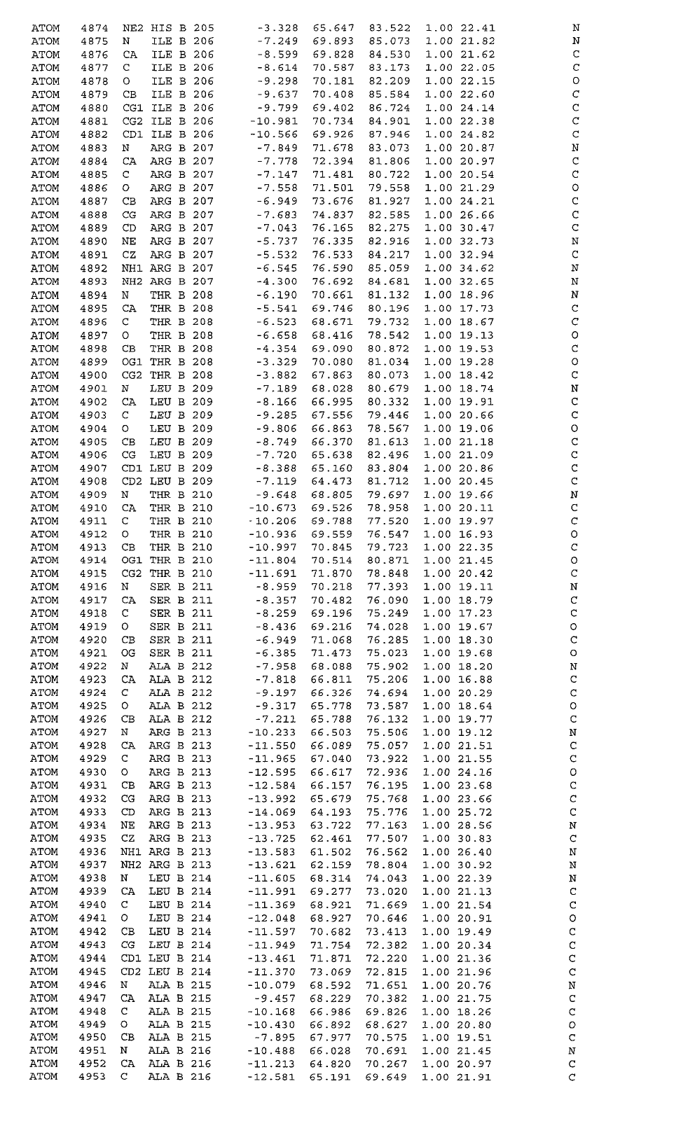

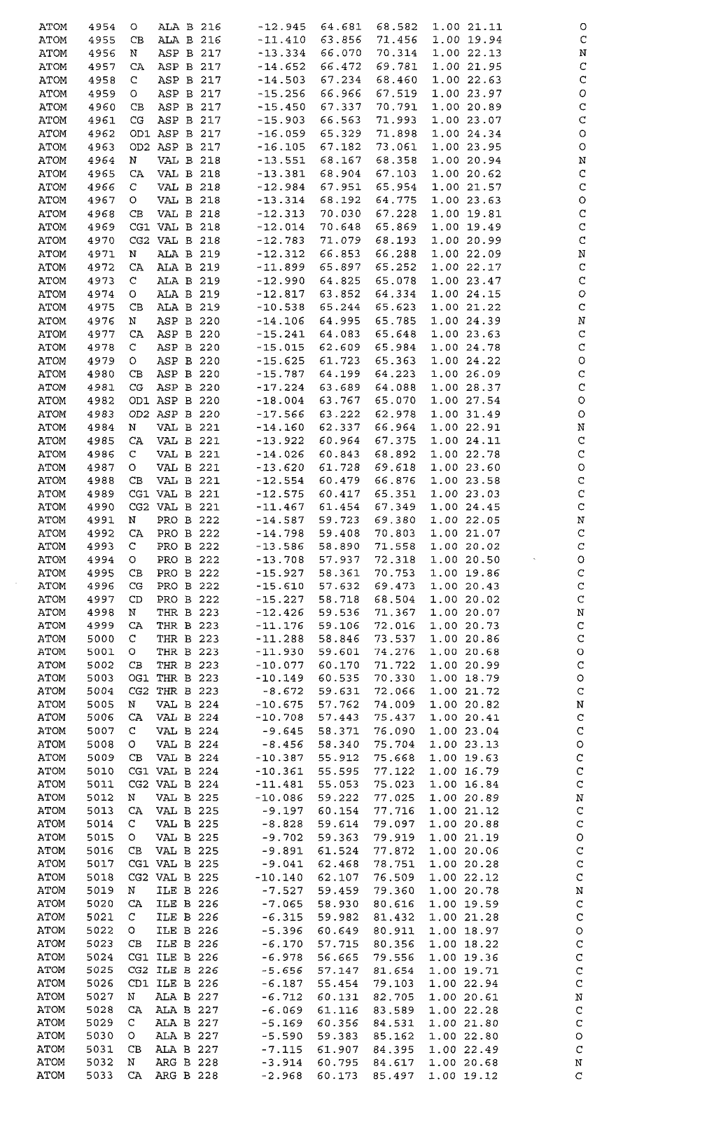

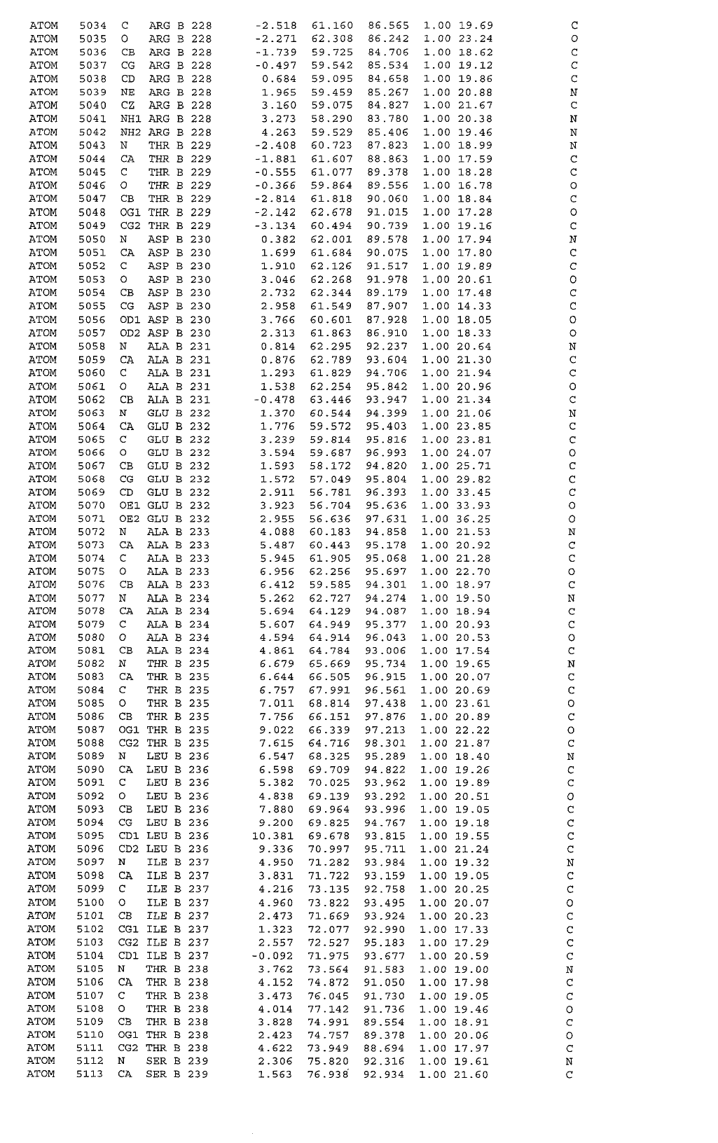

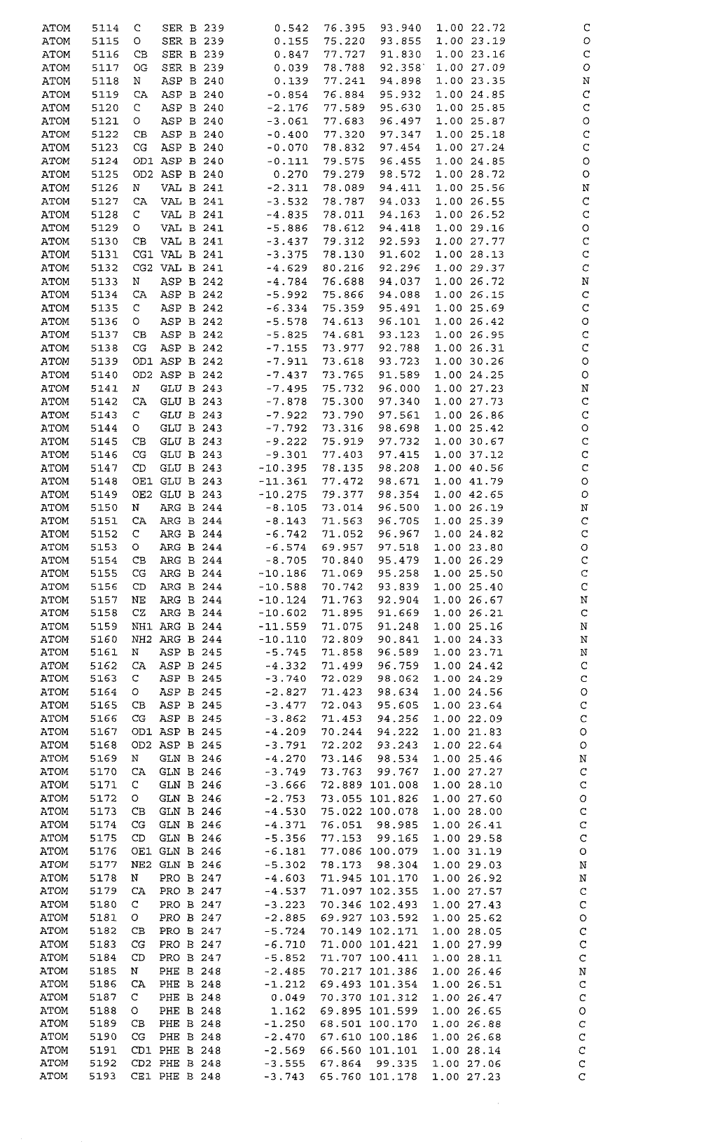

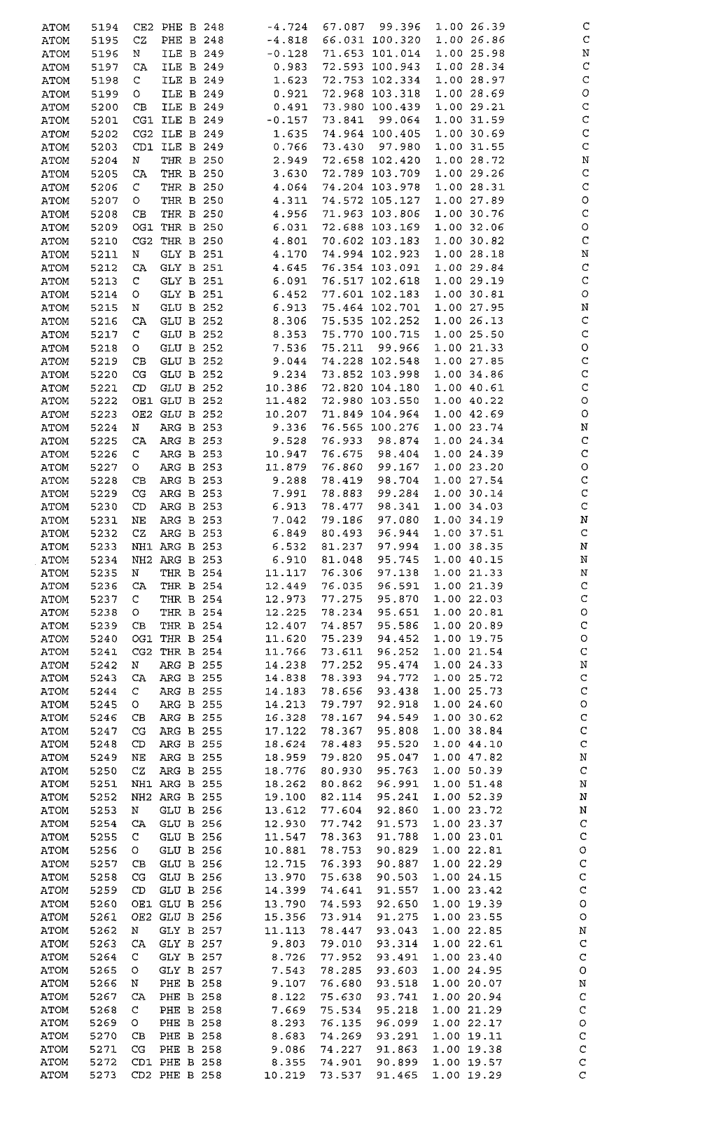

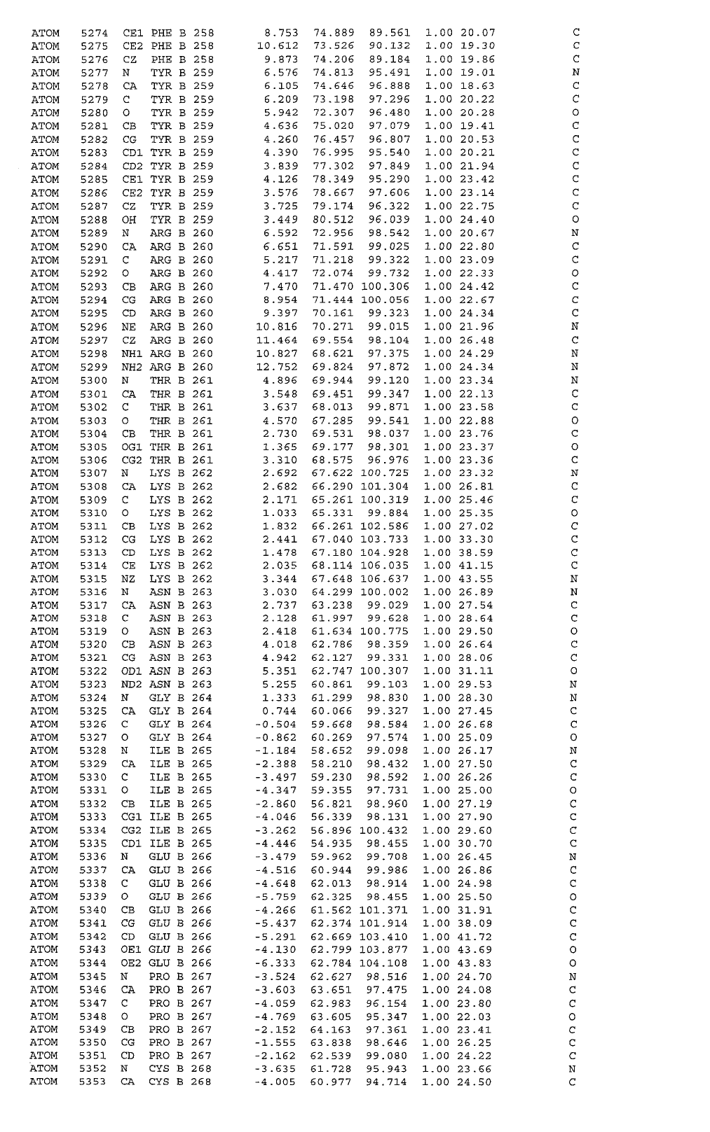

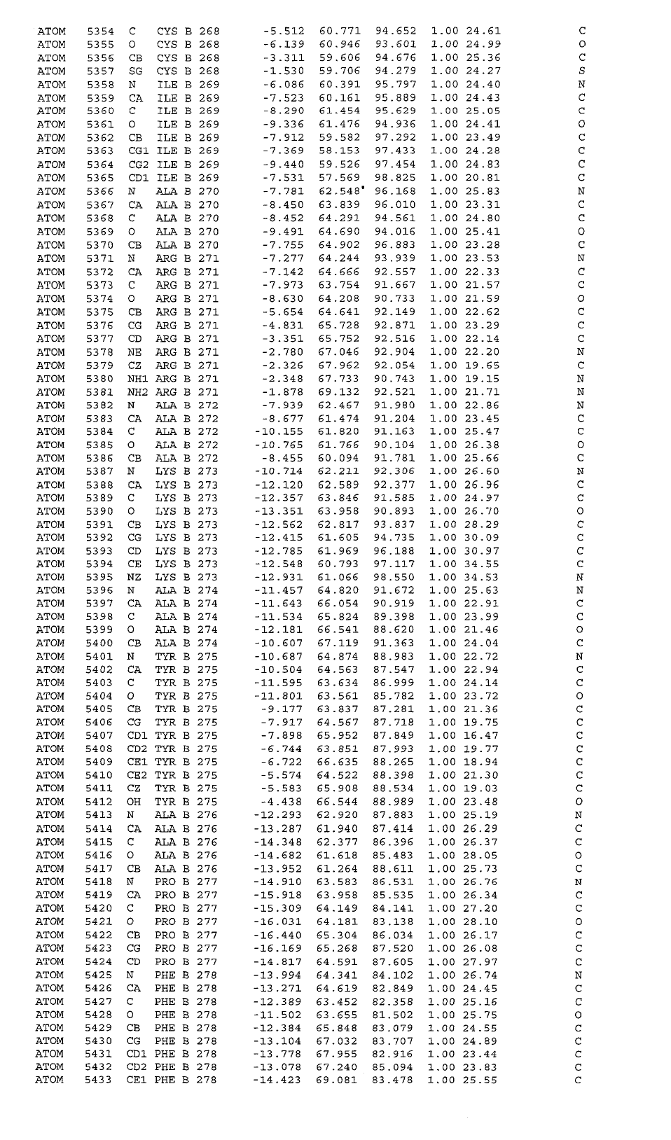

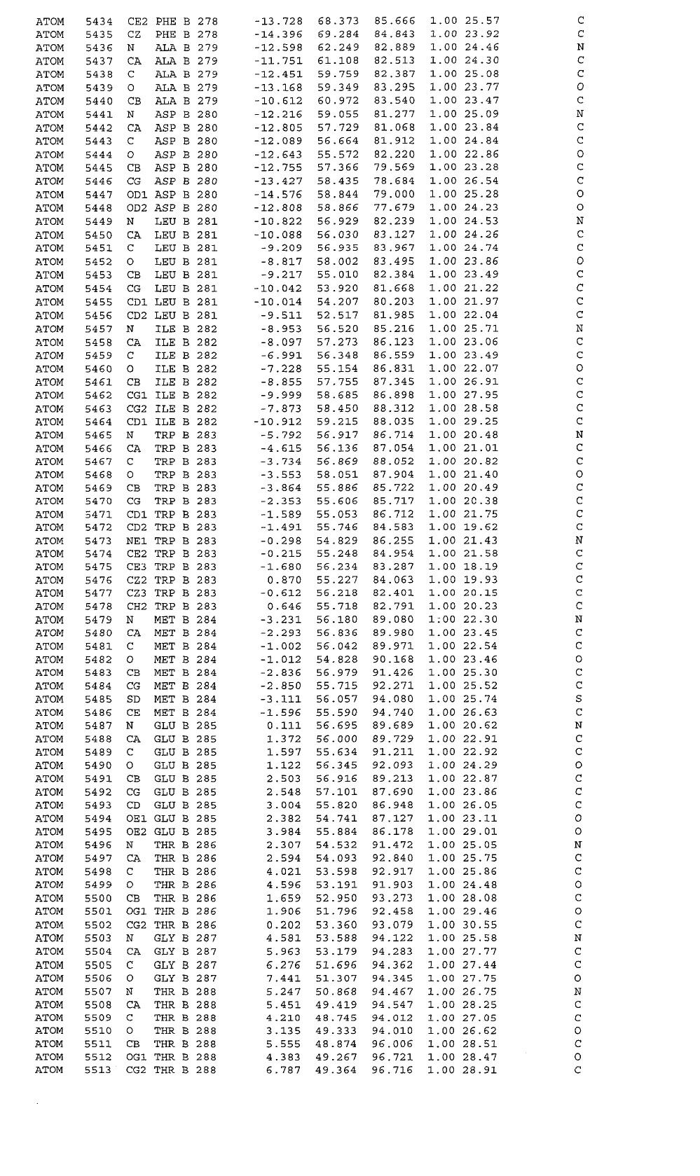

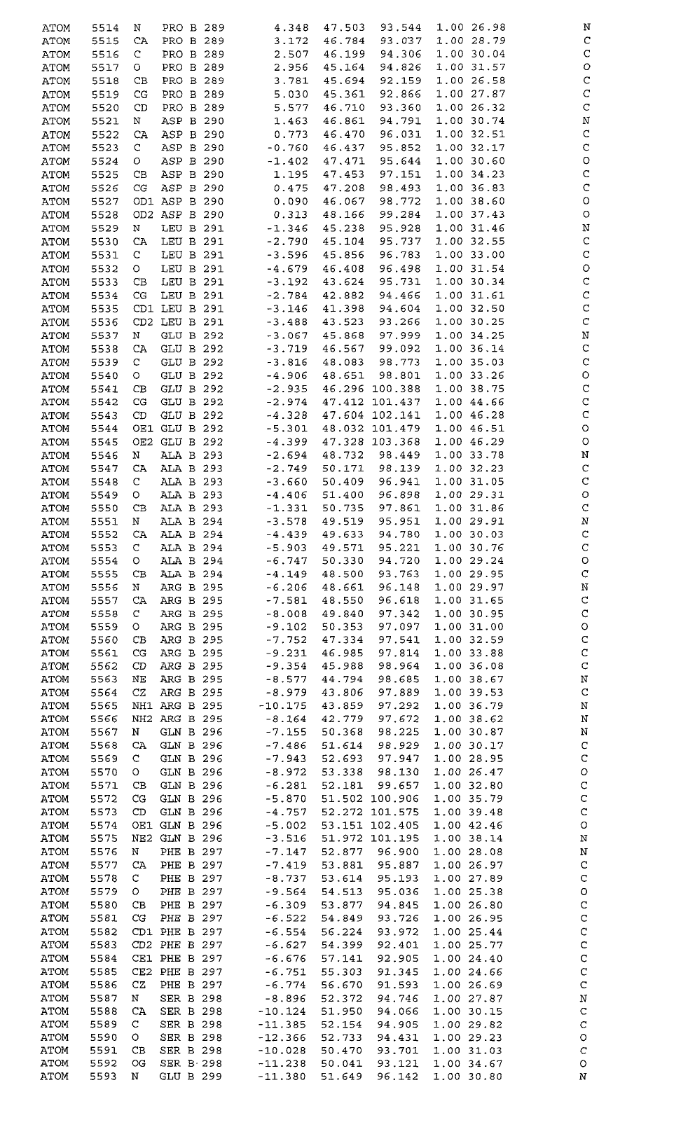

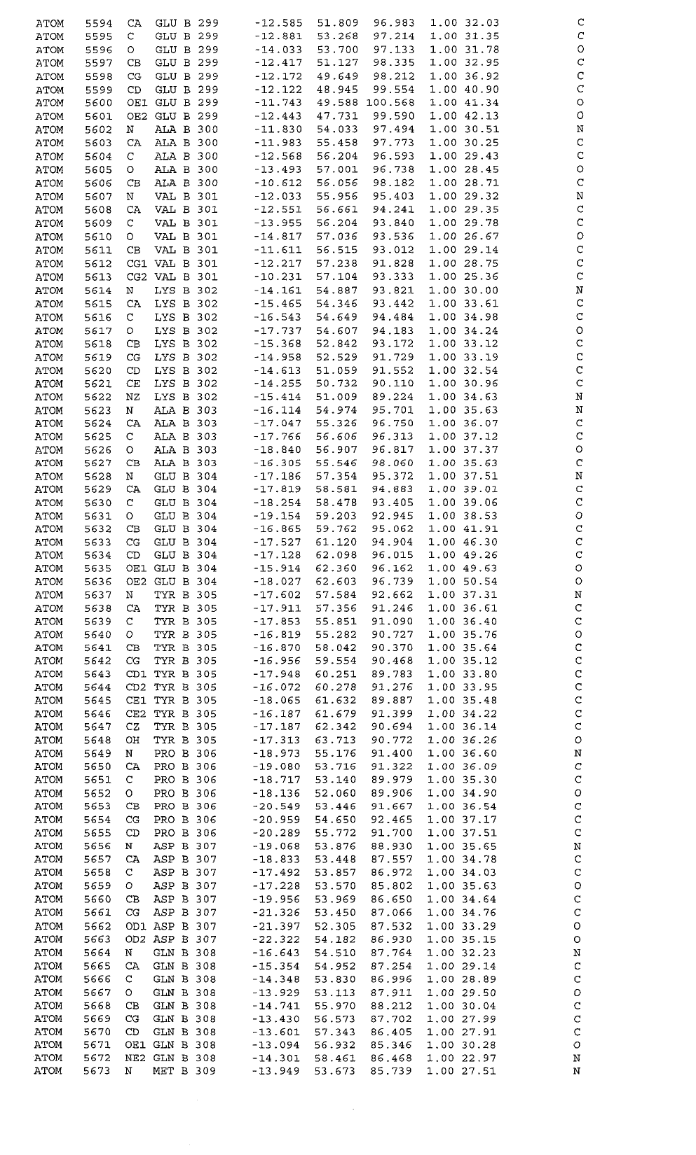

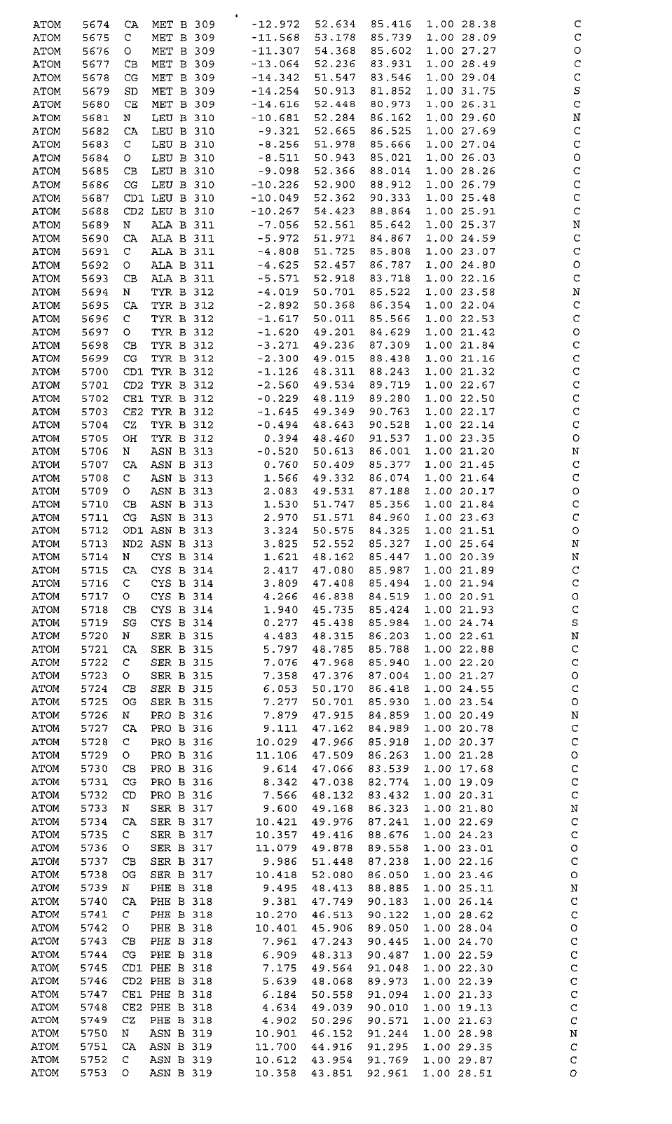

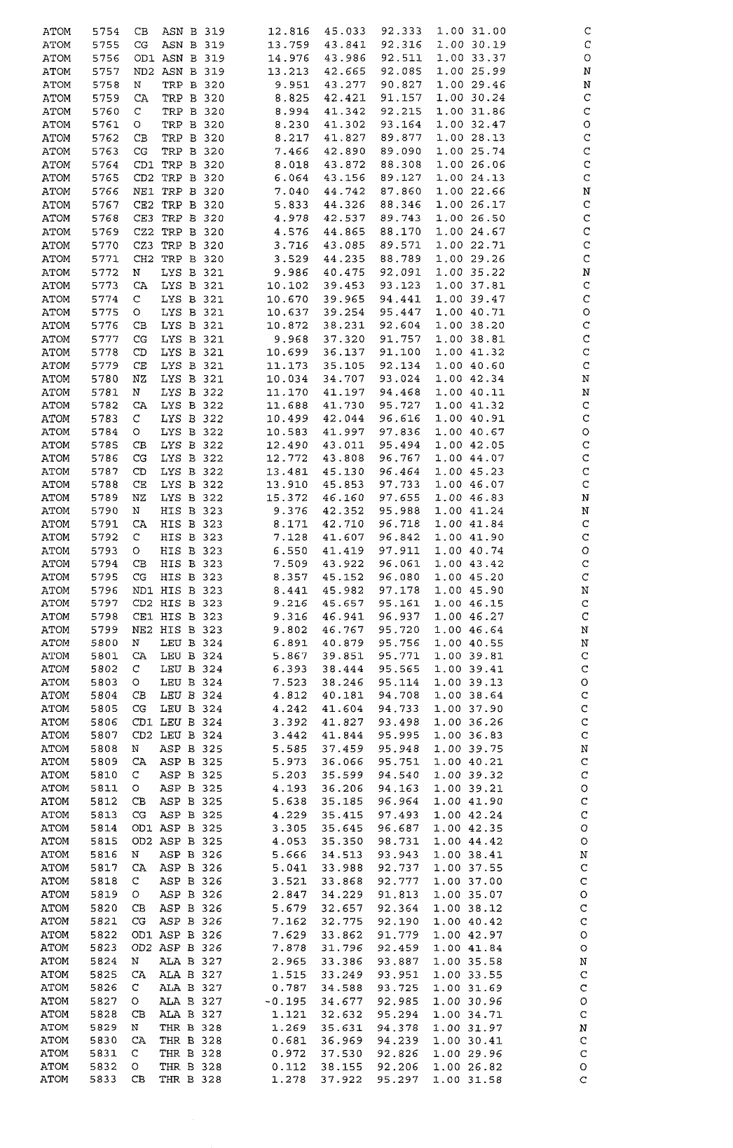

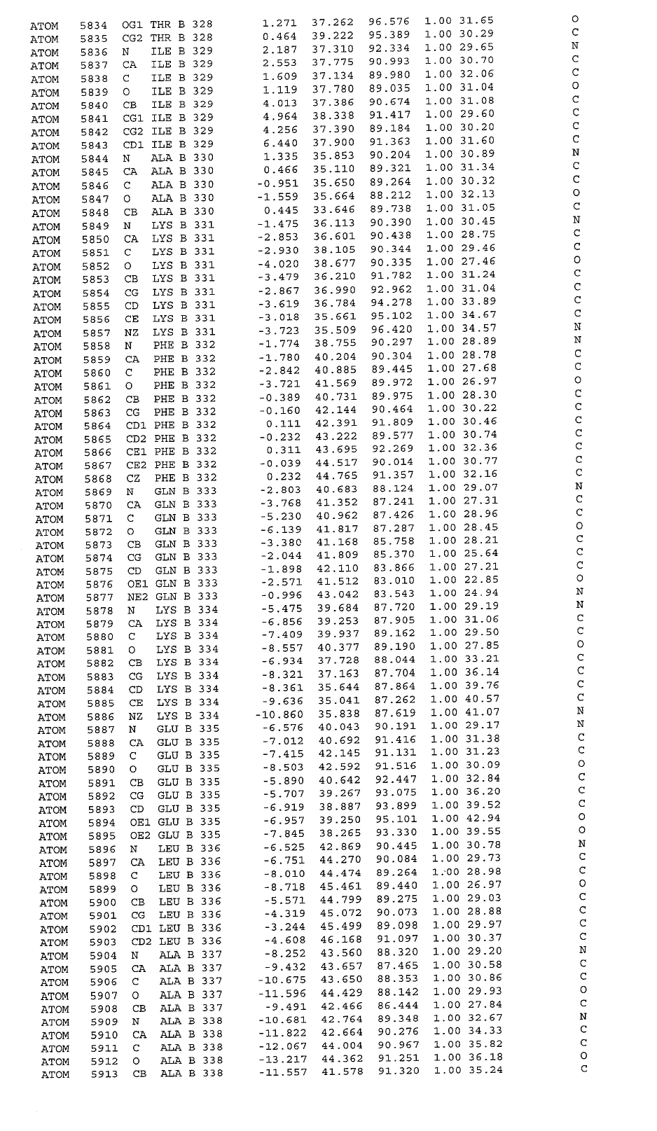

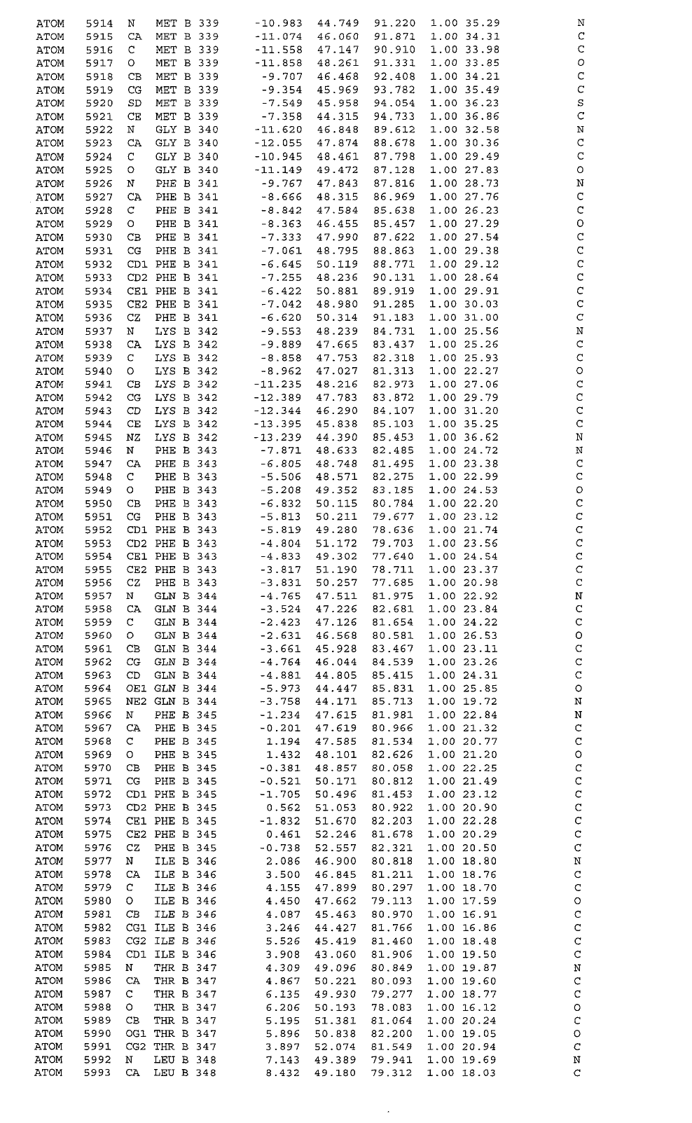

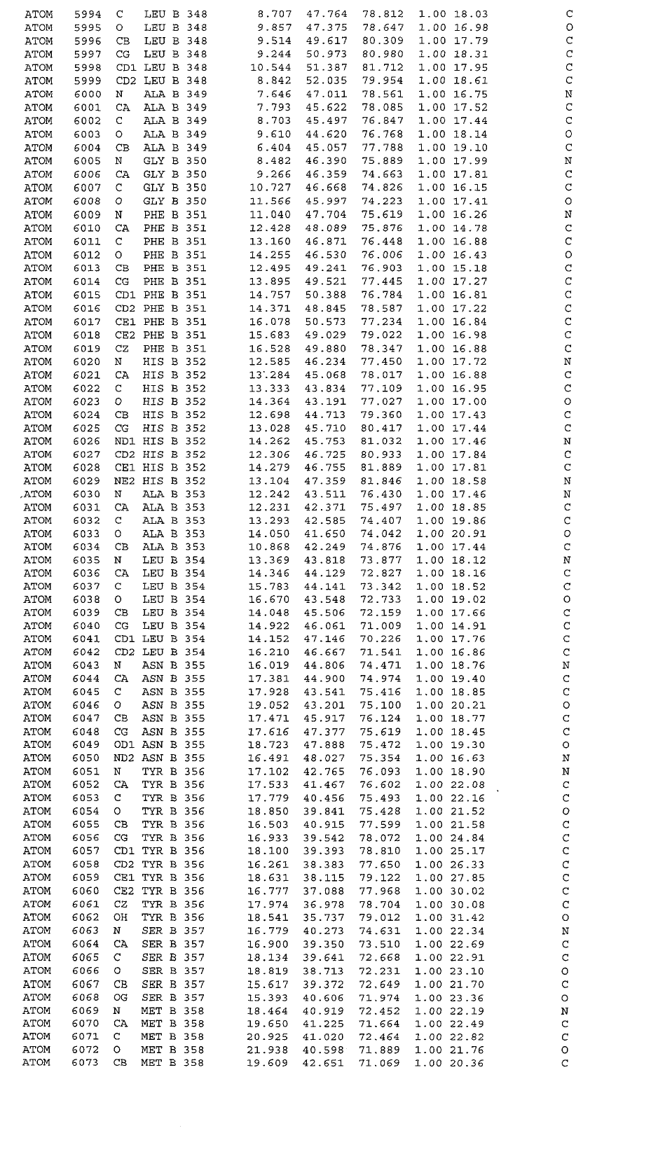

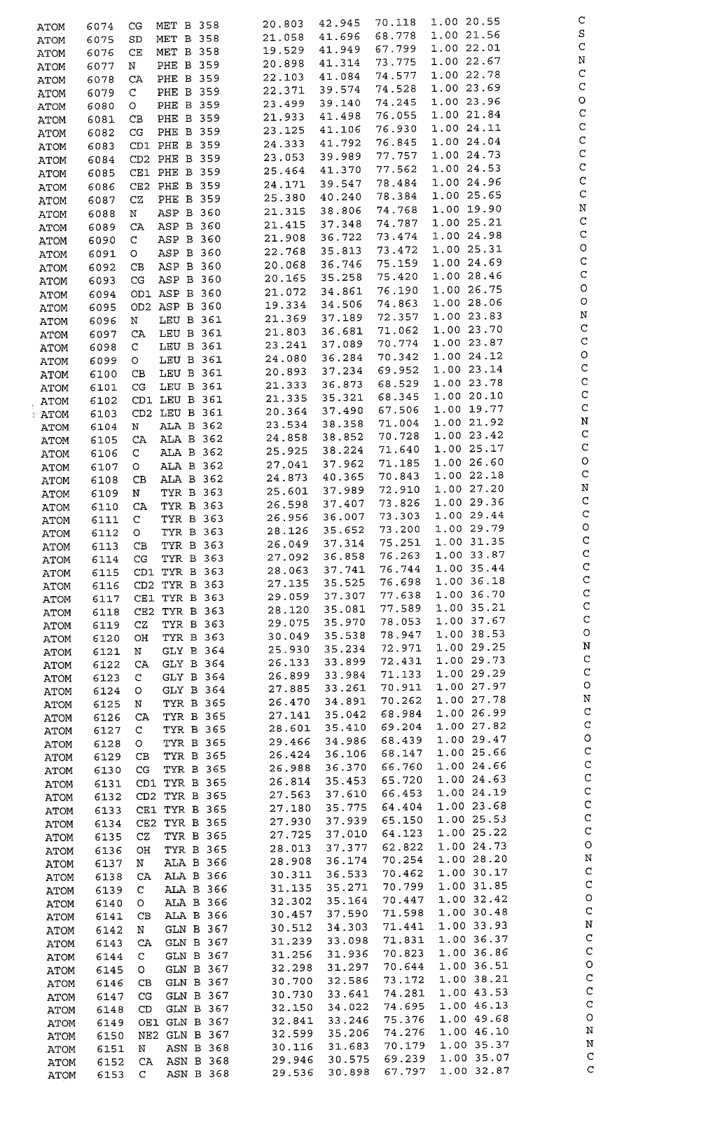

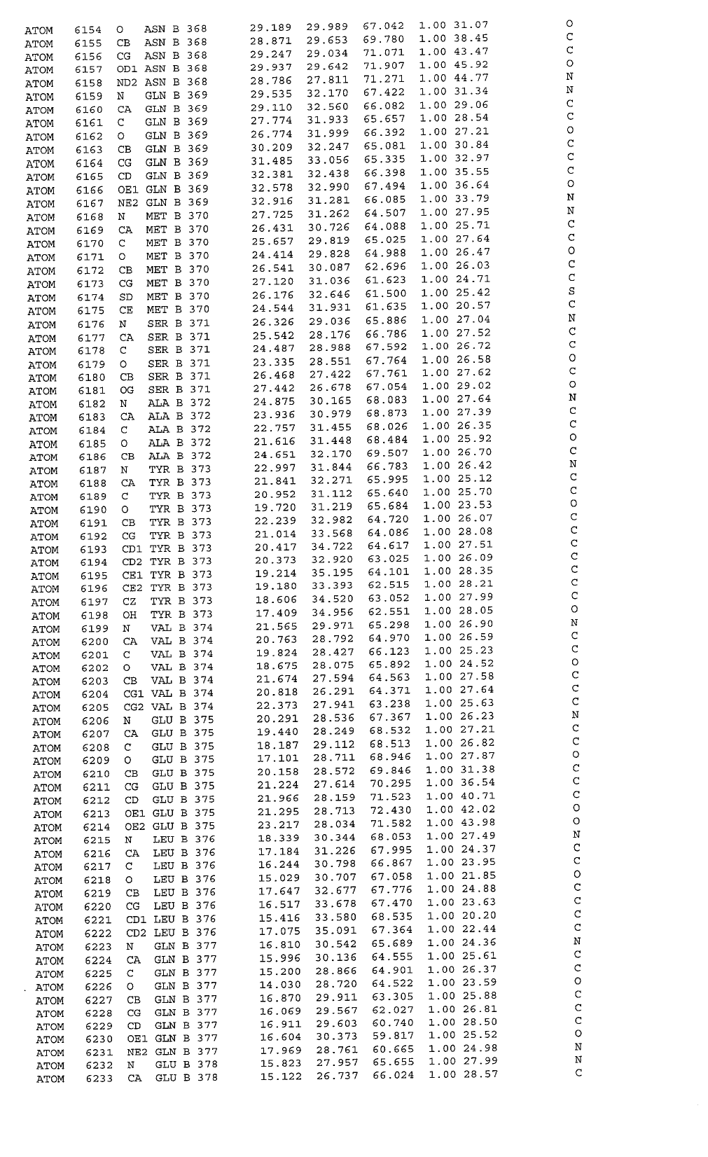

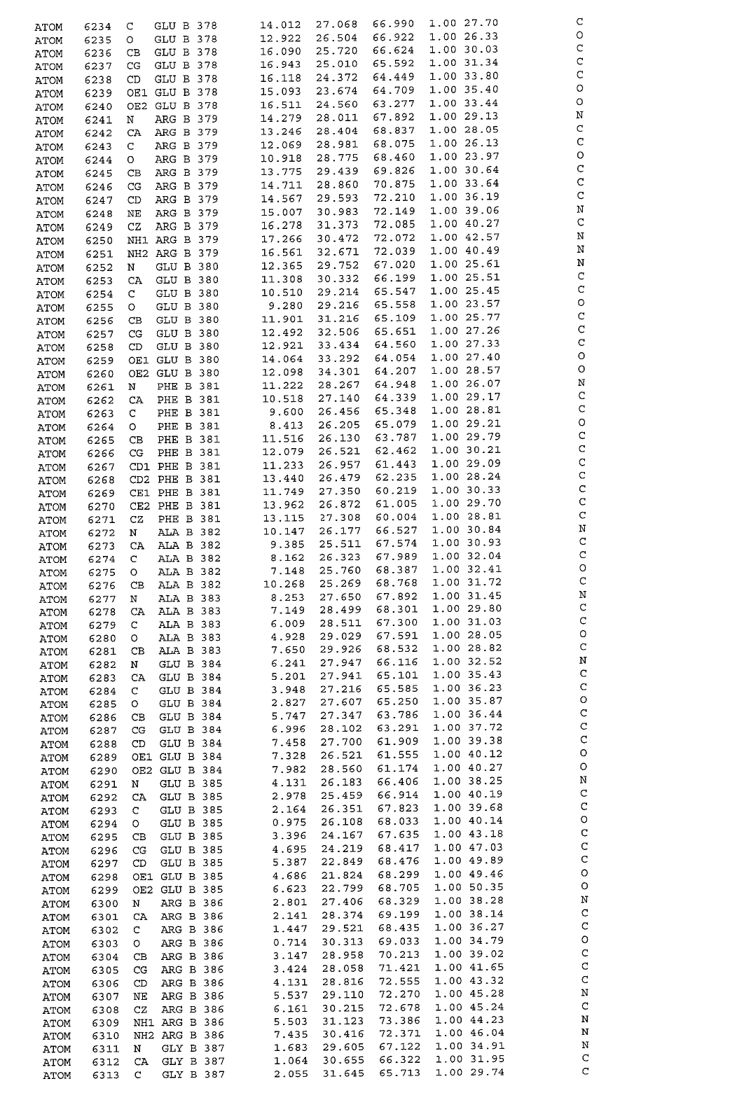

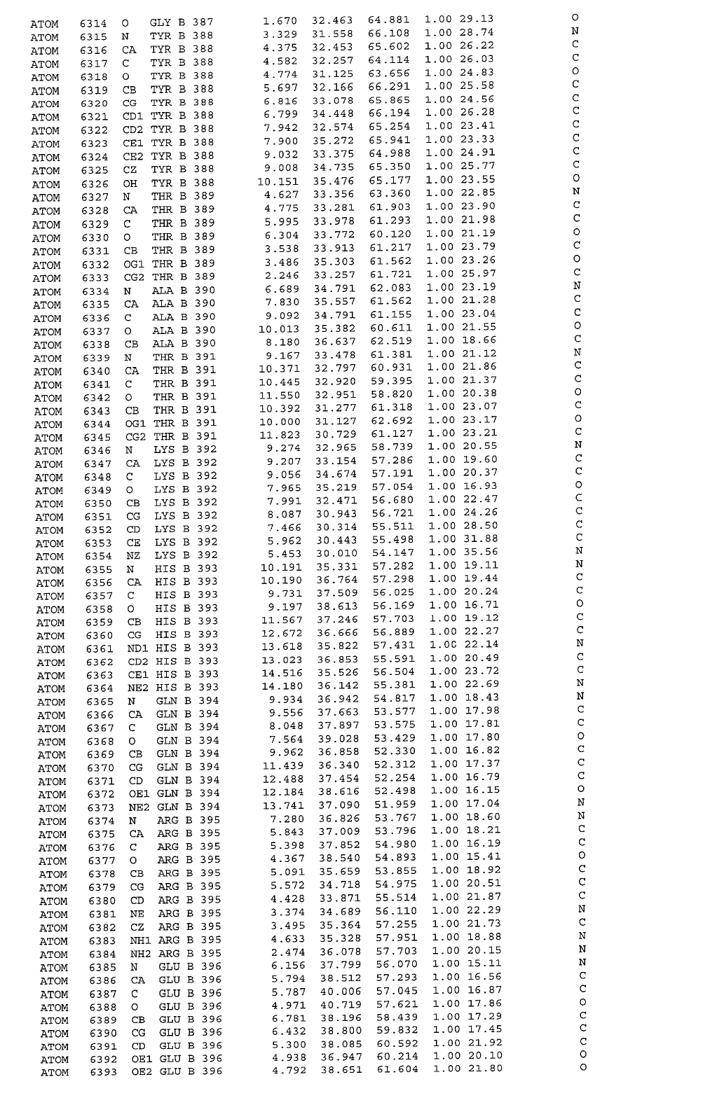

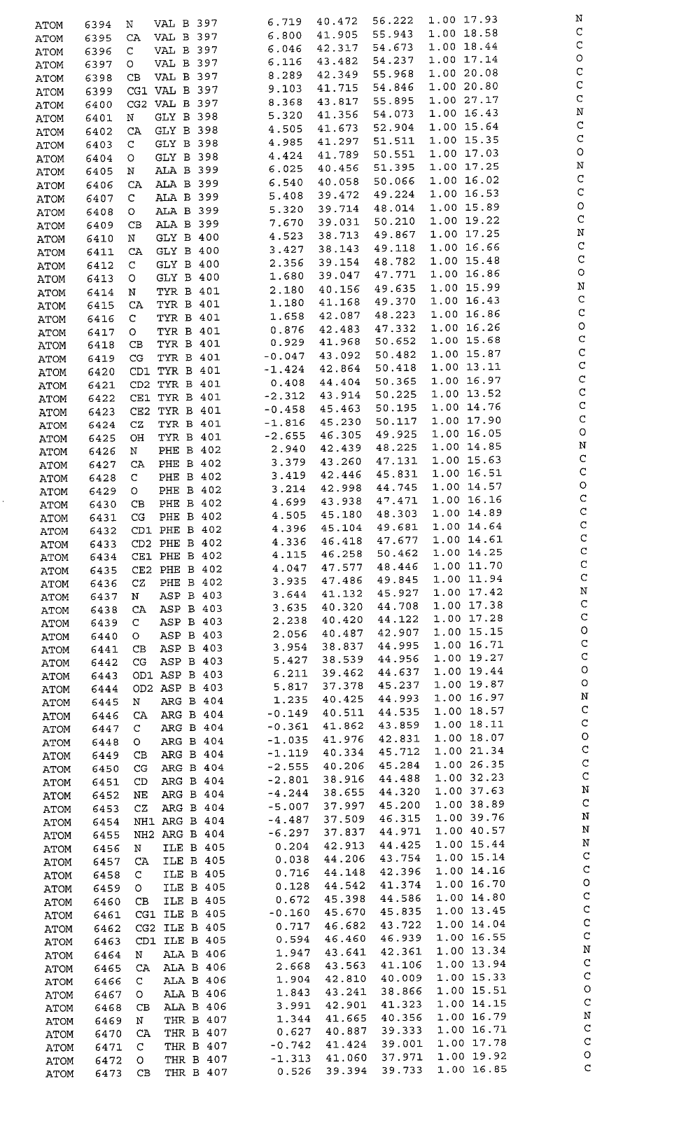

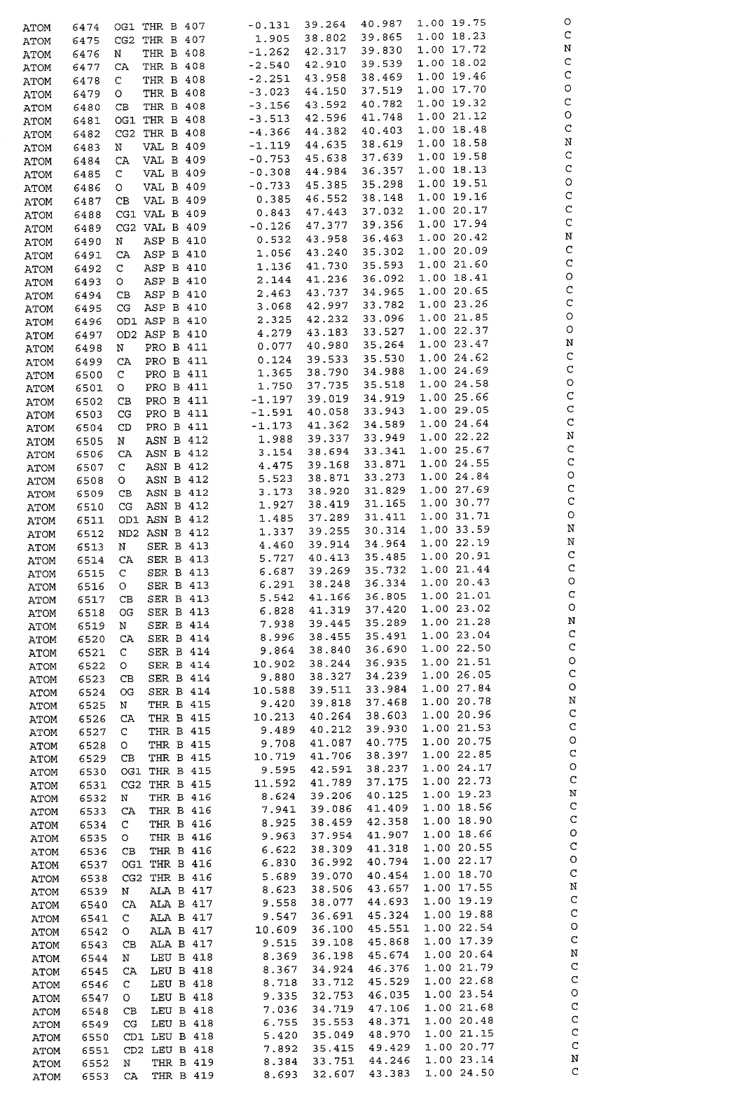

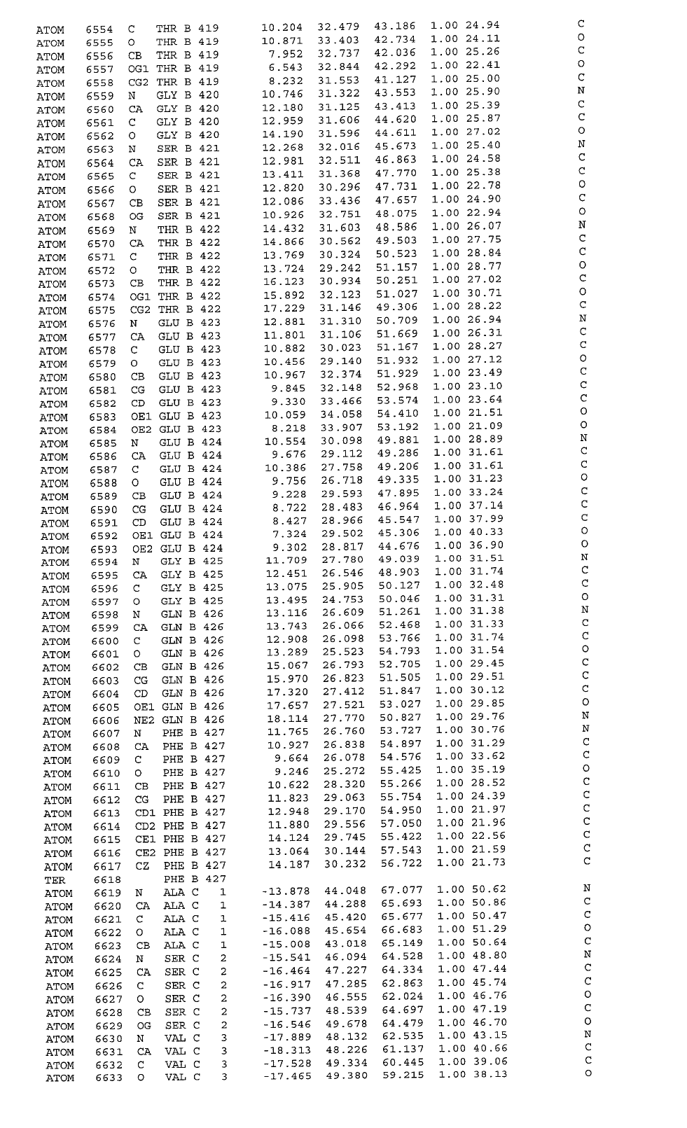

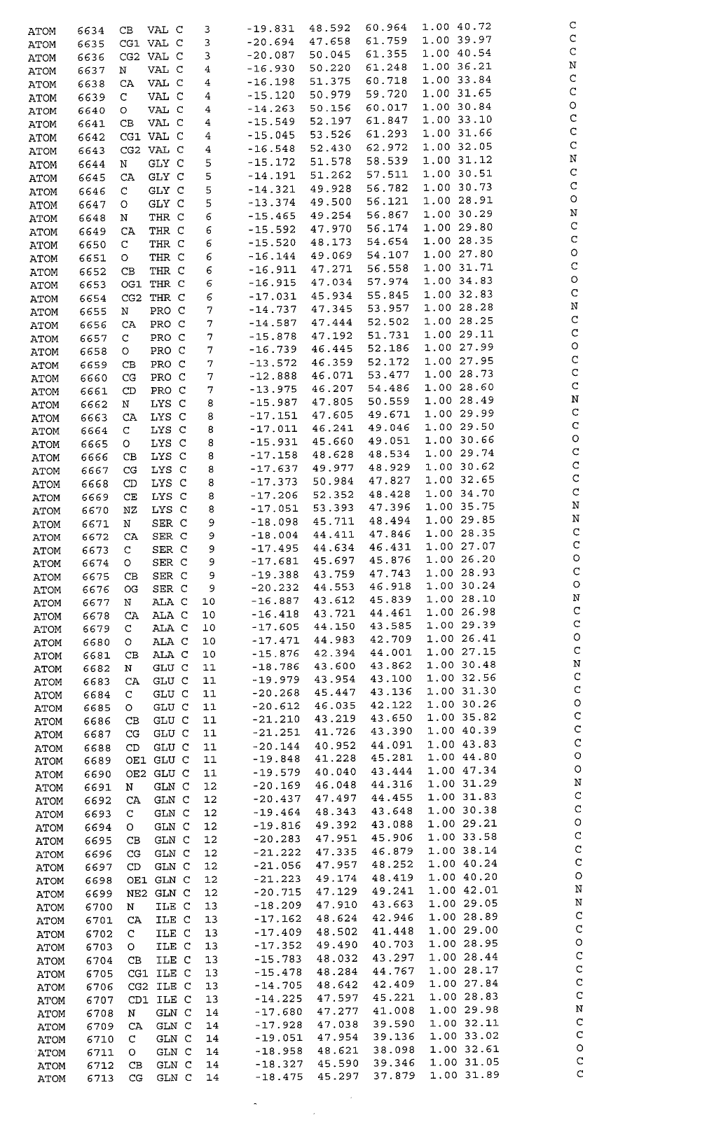

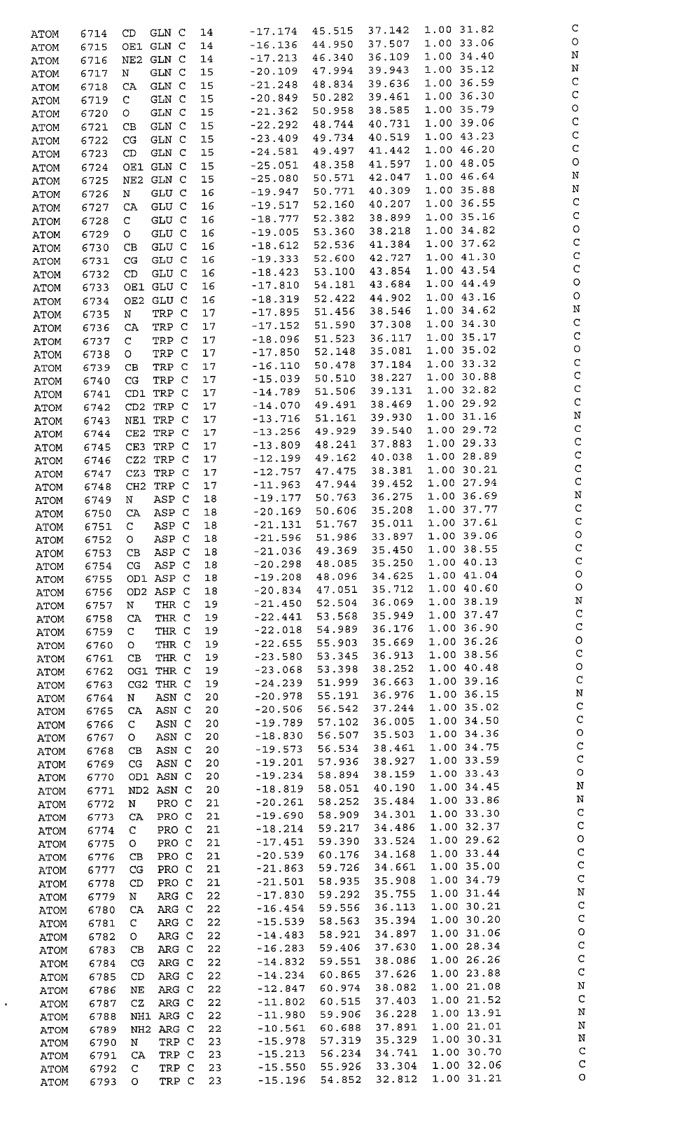

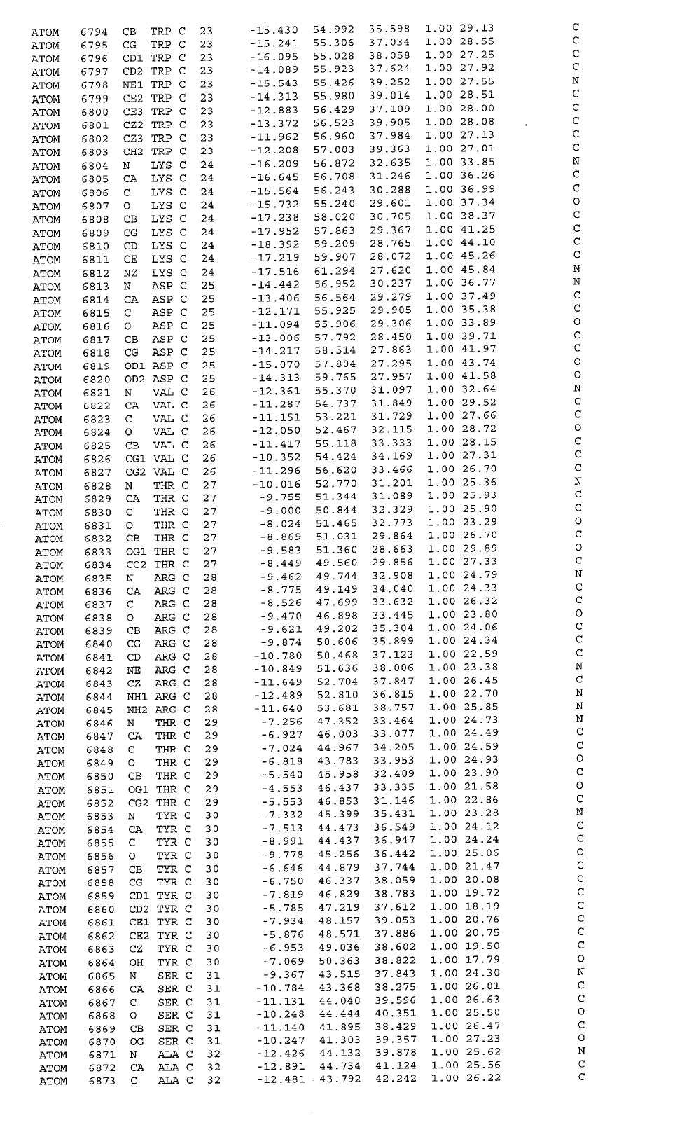

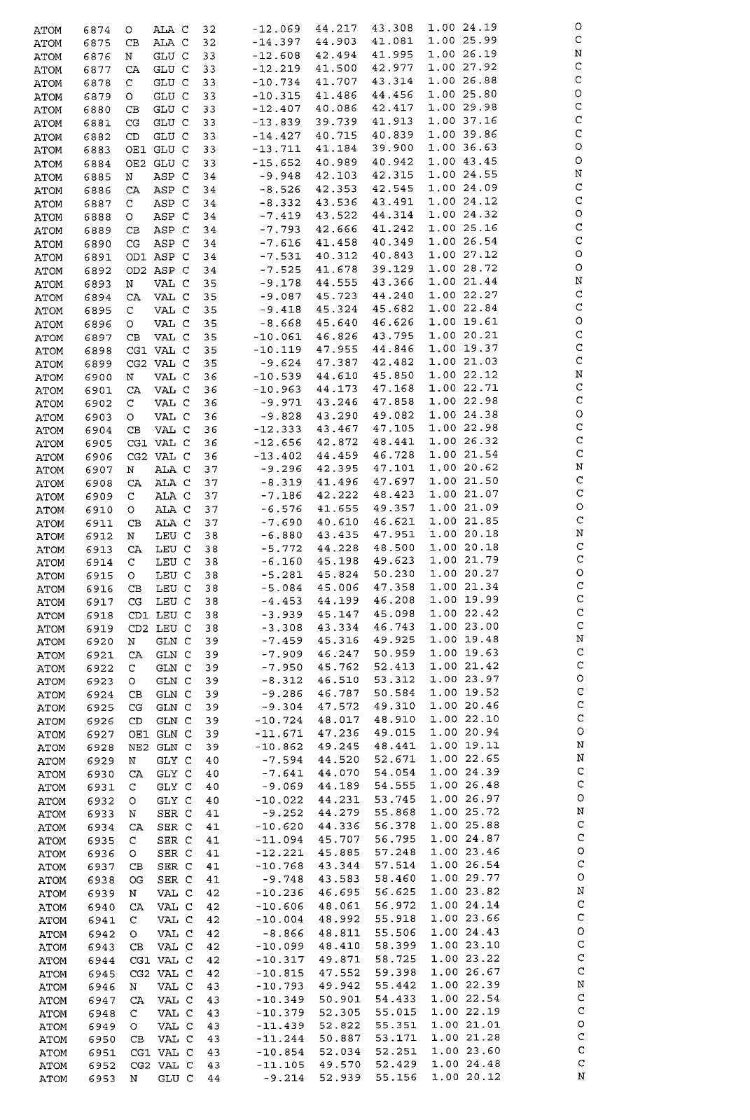

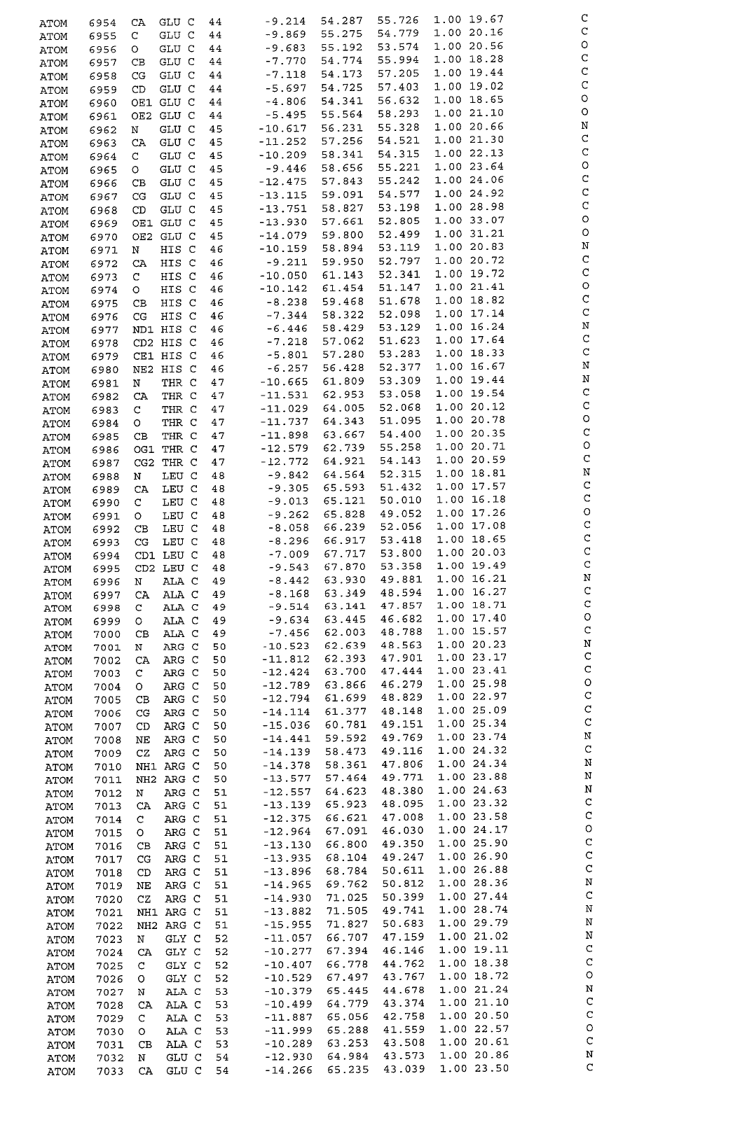

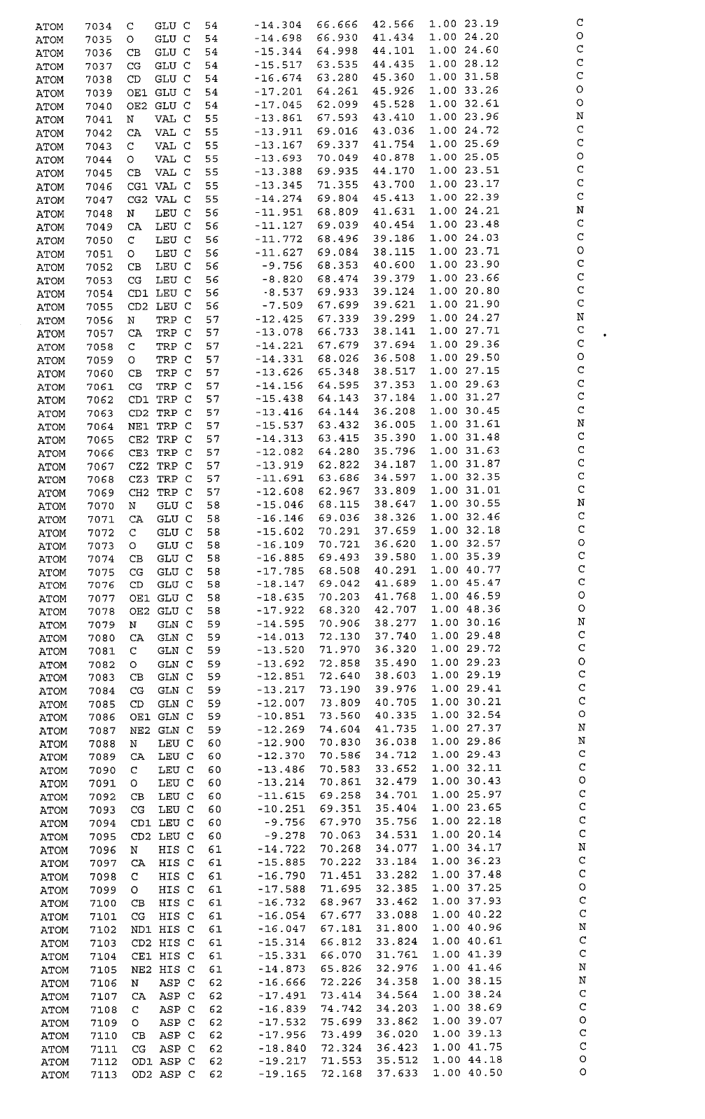

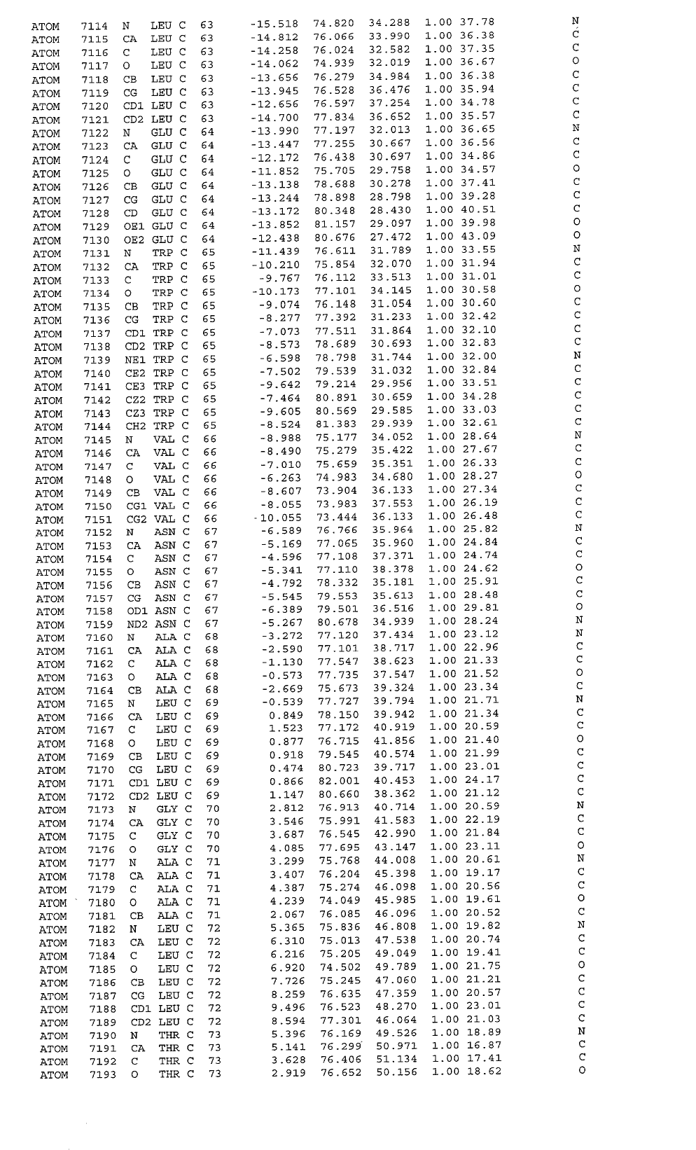

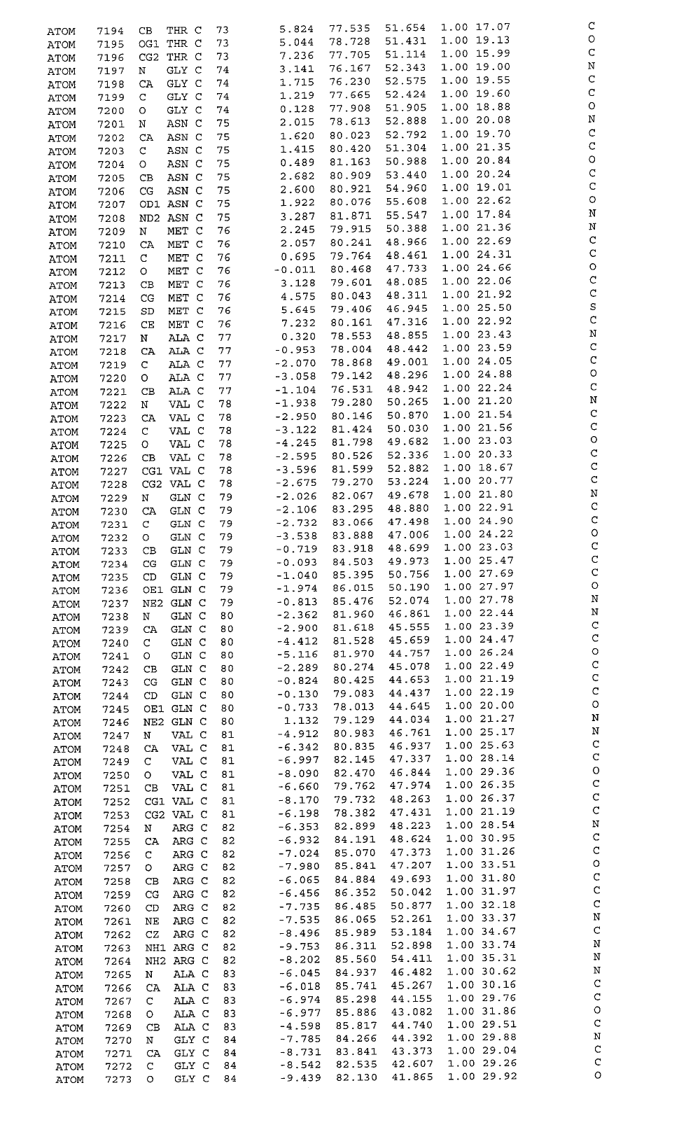

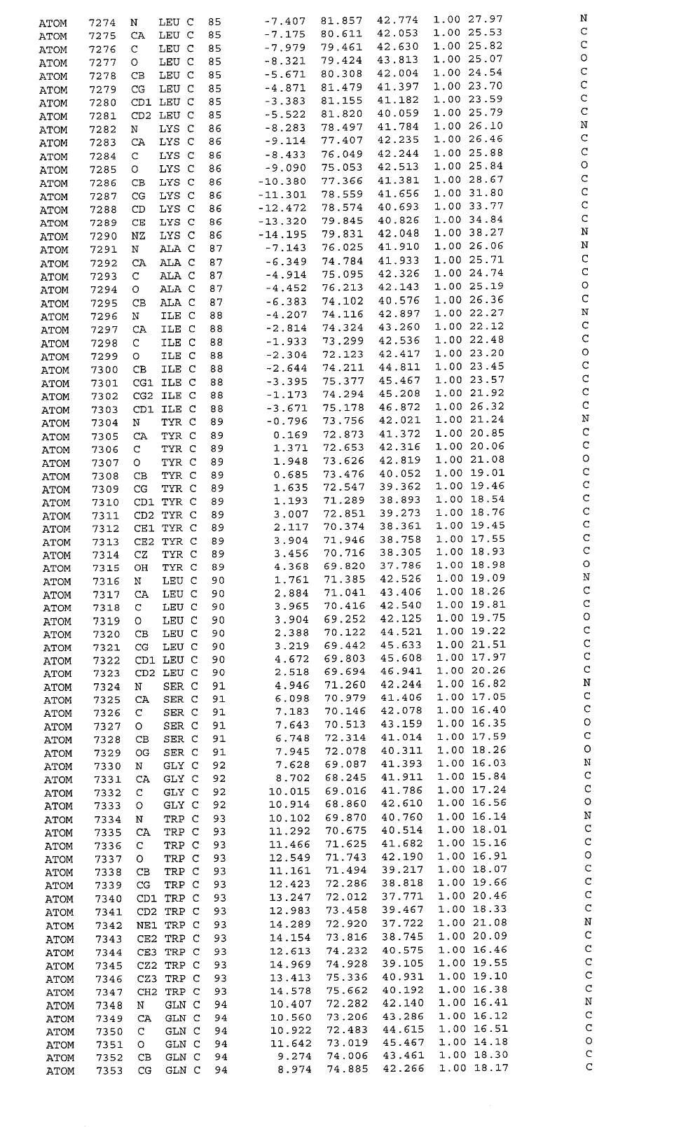

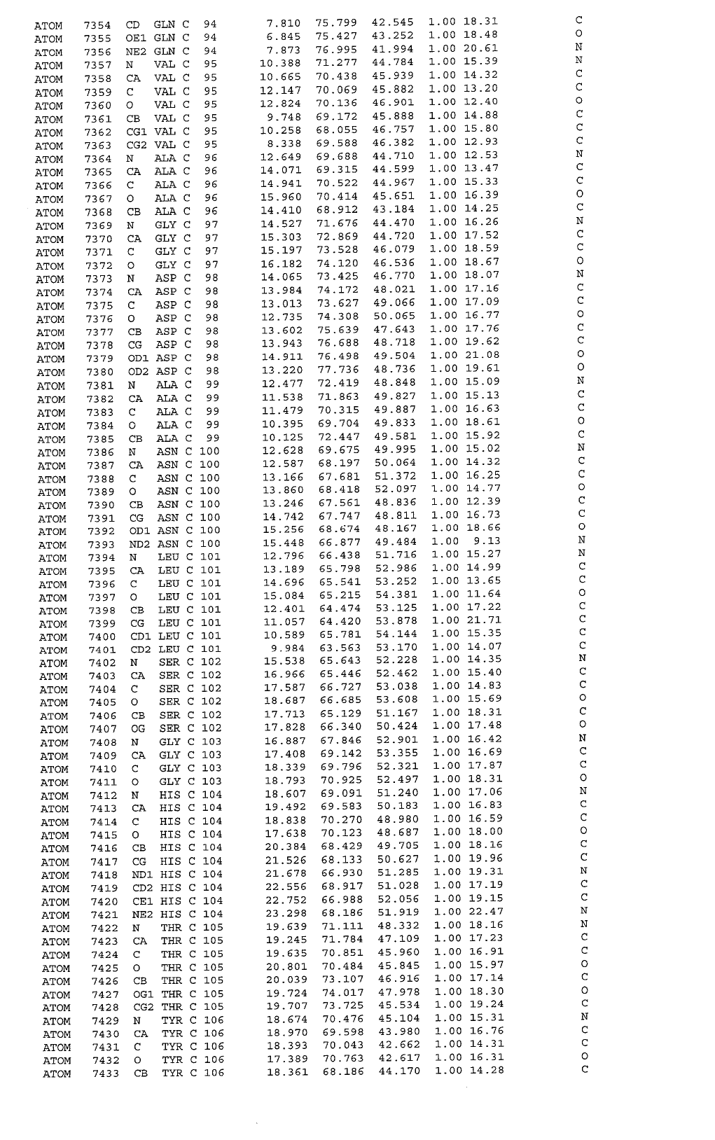

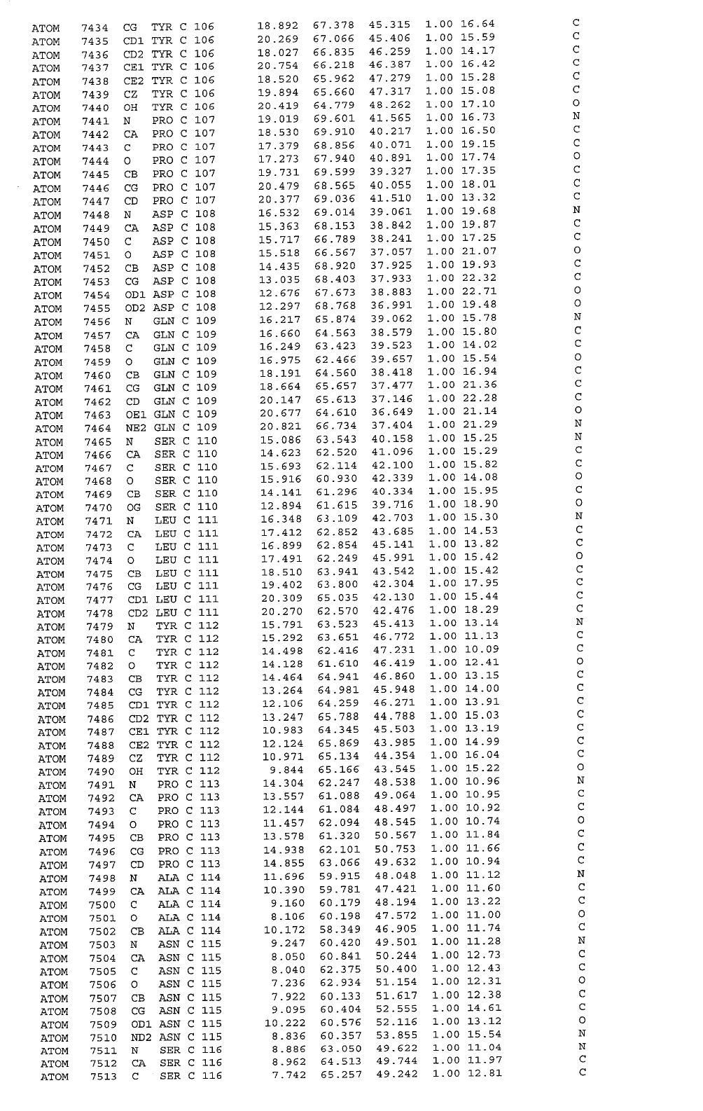

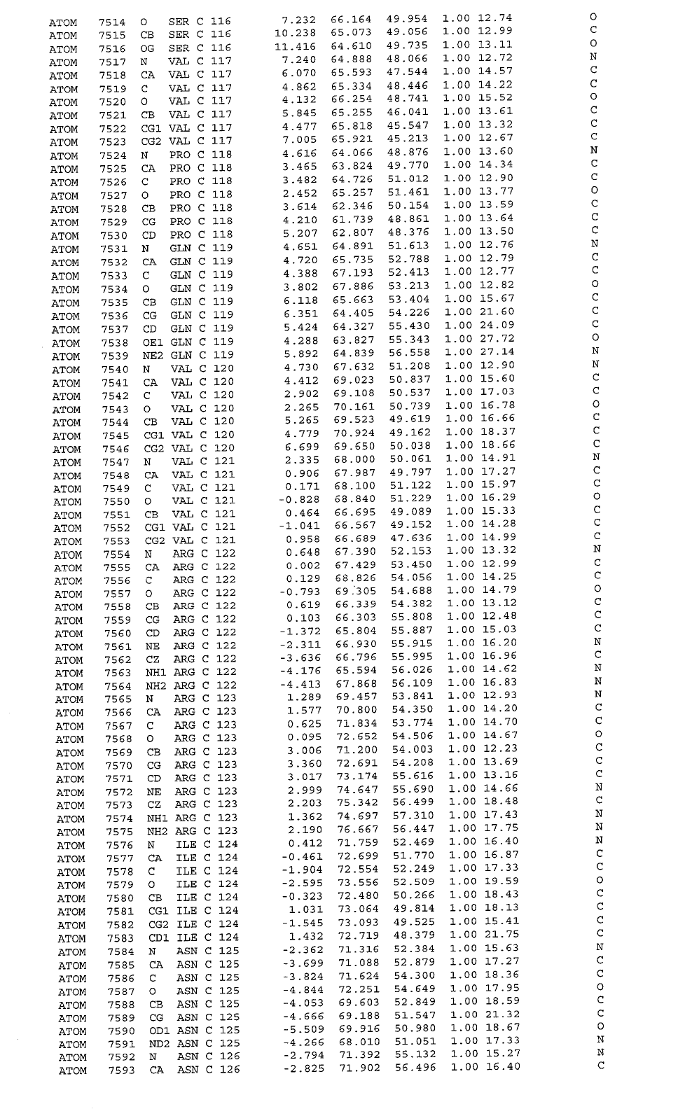

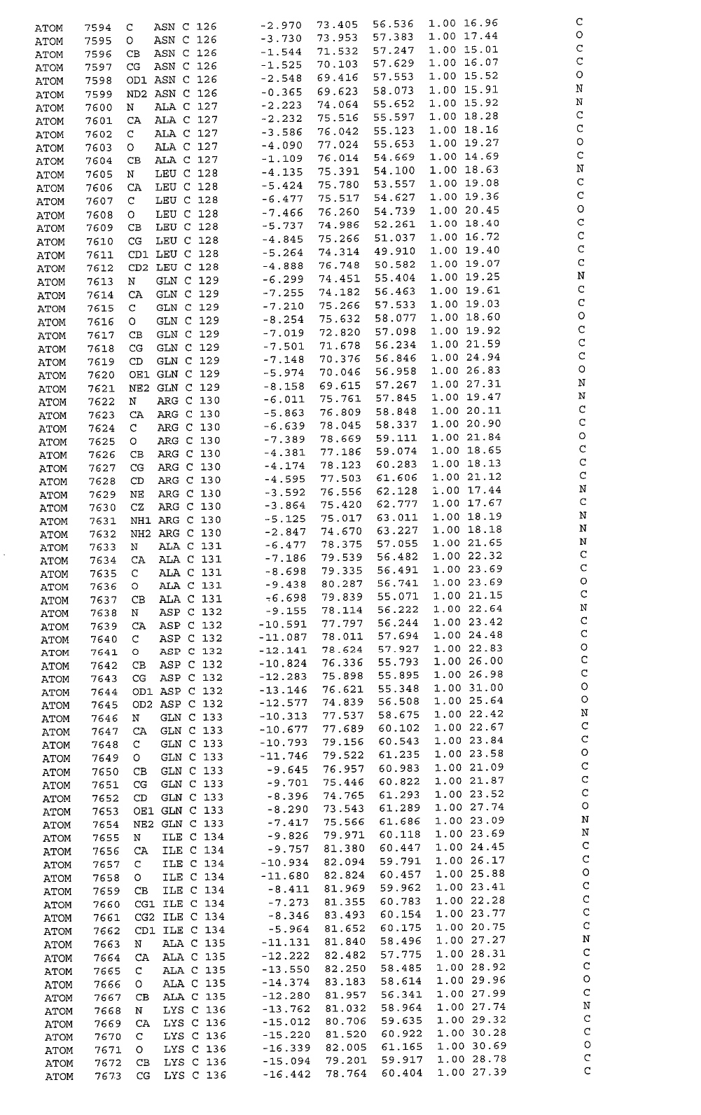

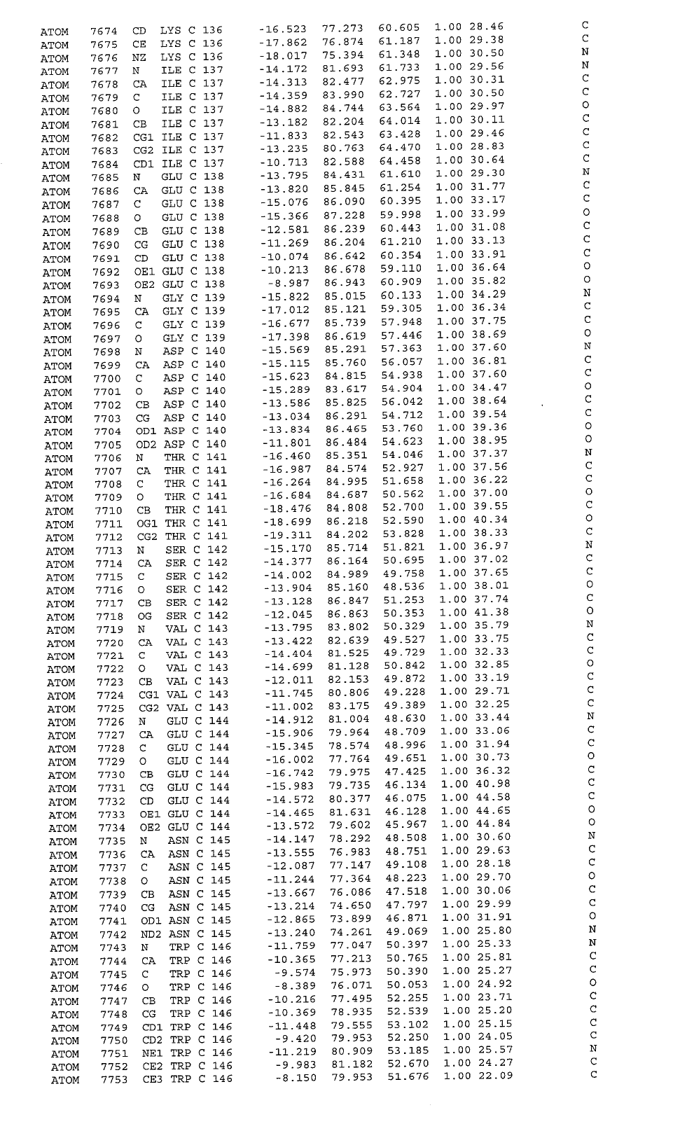

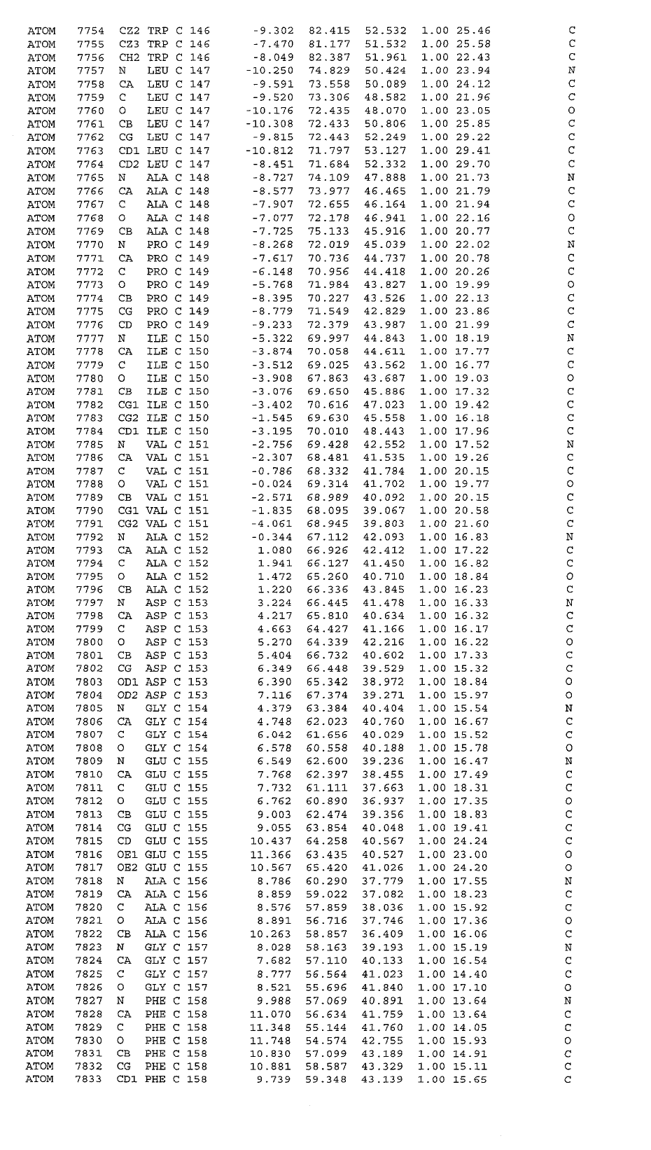

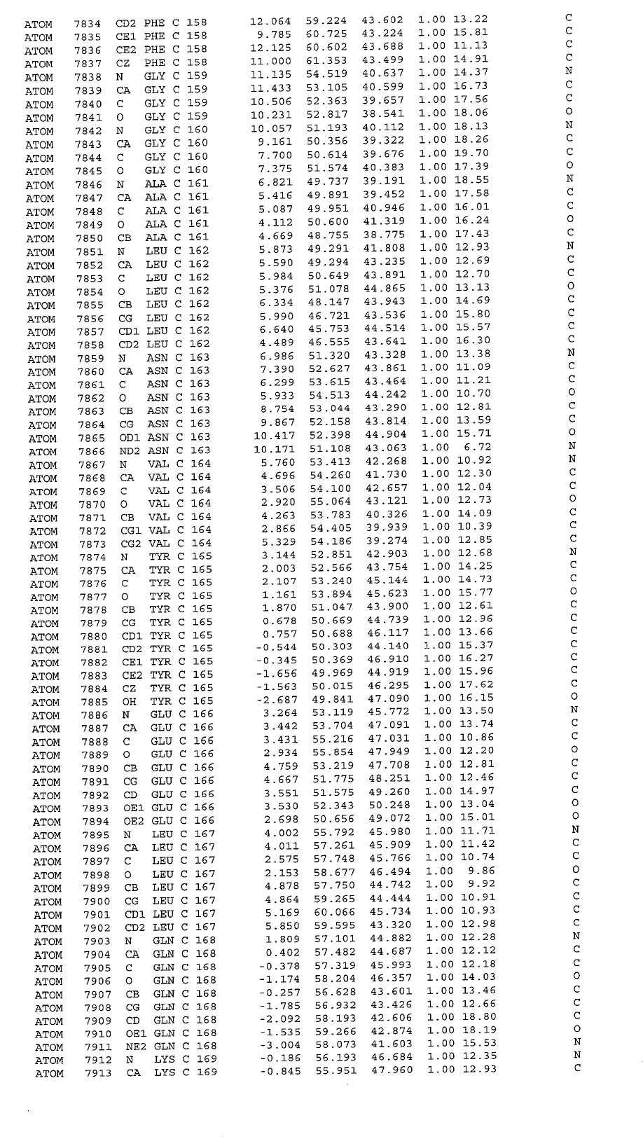

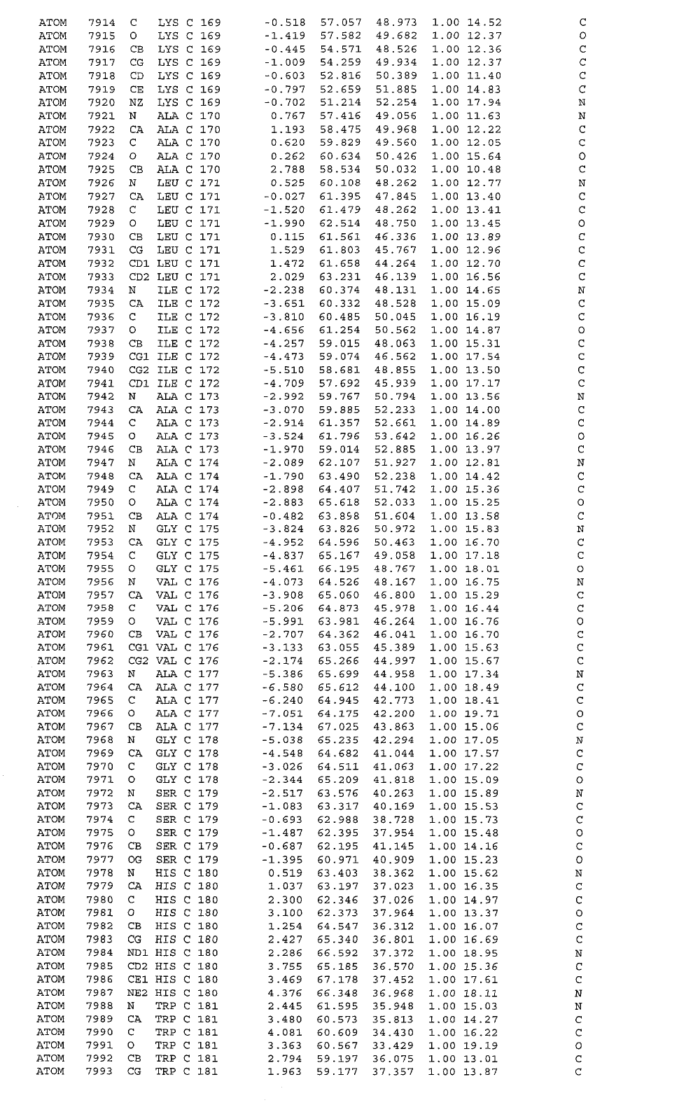

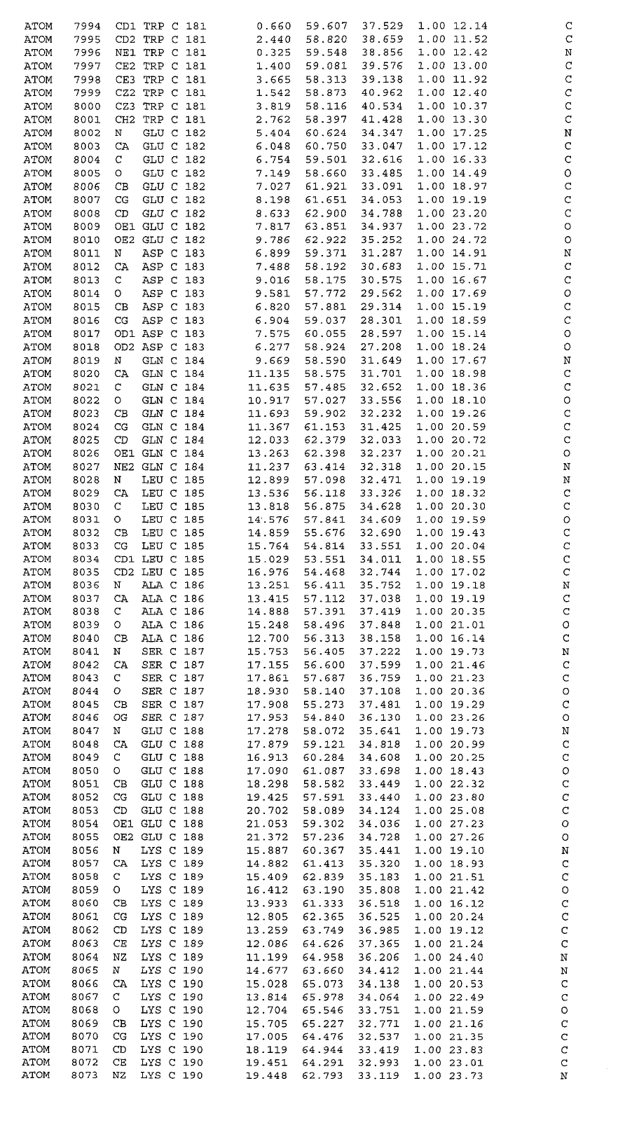

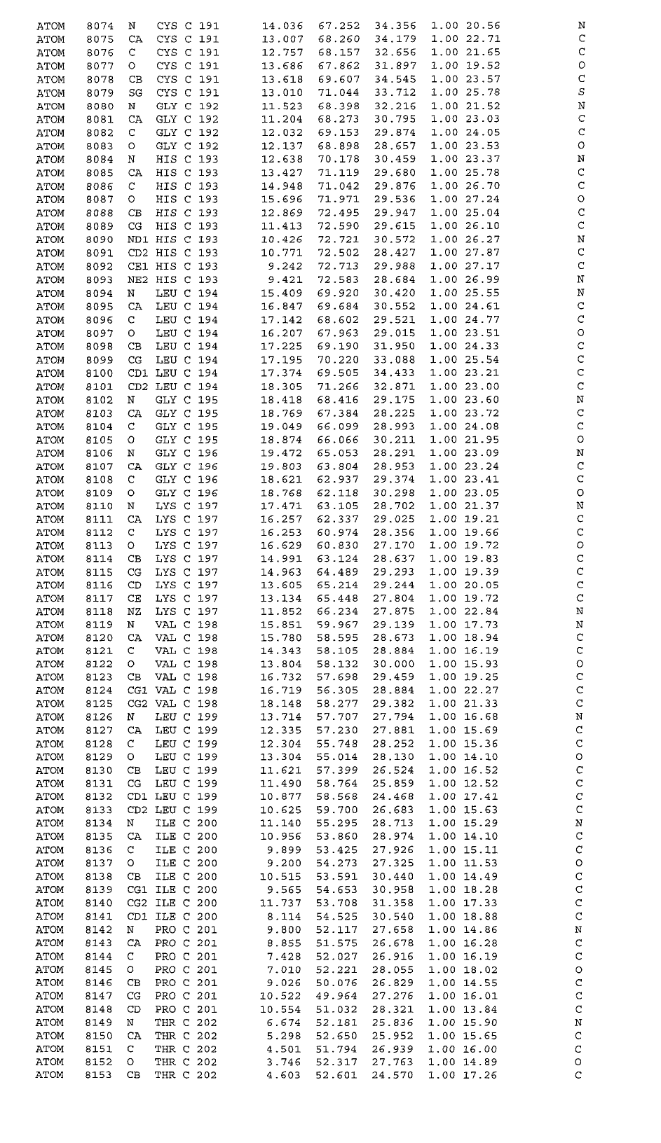

Inhibition of ICL by 3-bromopyruvate is accomplished via dehalogenation of the inhibitor to form a covalent adduct with active site nucleophile, Cys 191. The pyruvyl moiety occupies the site where the second carboxylate of succinate was located and forms hydrogen bonds with the side chains of His 193 ND1, Asn 313 ND2, Ser 315 OG, Ser 317 OG, Thr 347 OG1 and a solvent molecule (FIG. 4A and FIG. 4B). However, the orientation of the carboxylate group differs in the two cases, perhaps as a result of the covalent linkage with Cys 191 in the case of 3-bromopyruvate modified ICL. The residue Cys 191 adopts a conformation almost identical to Ser 191 in the C191S mutant, indicating that accommodation of the pyruvyl moiety did not require any additional rearrangement of the active site residues. In this complex, the glyoxylate binding site is occupied by solvent molecules that coordinate the Mg²⁺ ion.

4. Inhibitor Induced Conformational Changes. Comparison of the structures of the native and inhibitor bound forms of ICL showed large conformational changes (FIG. 4C and FIG. 4D) in two regions that control access to the active site. The first region is an active site loop (residues 185-196) that contains the ICL signature sequence (K₁₈₉KCGH₁₉₃) and the second region consists of the last 18 residues (residues 411-428) at the C-terminal end of the adjacent subunit. In the ‘open’ conformation of the apoenzyme, cysteine (Cys 191) in the active site loop is relatively far from other catalytic residues and the binding site is highly solvent accessible. Poor electron density was observed for residues His 193 and Leu 194 for one of the subunits in the asymmetric unit, suggesting that the active site loop is flexible in the ‘open’ conformation.

Upon binding of inhibitor, the loop moves by 10-15 Å and adopts the ‘closed’ conformation (FIG. 4C and FIG. 4D). Unlike the ‘open’ conformation of the free enzyme, access to the catalytic site in the inhibitor bound ‘closed’ conformation is completely blocked by the active site loop (residues 185-196). Closure of the active site loop invokes a movement of residues 411 to 428 of the adjacent subunit (FIG. 4C and FIG. 4D). Whereas the last 11 residues were somewhat disordered and extend into solvent in the apo enzyme crystal, clear electron density was observed for all except residue 428 of the C-terminus in the inhibited enzyme crystal. In the ‘closed’ conformation, residues 411-427 occupy the space created by closing of the active site loop. In the resulting orientation, the C-terminus lies on top of the active site loop, locking it into the catalytic conformation. This two-step conformational rearrangement appears to be triggered by a 2.5 Å movement of a Mg⁺² ion which is bound in a highly electronegative depression formed by Asp 108, Asp 153, Glu 155 and Glu 182 in the apo enzyme. Binding of succinate appears to trigger the movement of the Mg⁺², allowing Lys 189 of the active site loop to form electrostatic interactions within this region, and facilitating closure of the active site loop over the bound substrates.

The inventors reason that successful drug discovery may involve pinpointing interactions that are important, or even critical, in obtaining the closed conformation. Occurrence of loop closure only when the succinate binding site is occupied by either nitropropionate or the pyruvyl moiety suggests that presence of succinate in addition to glyoxylate might be required to induce loop movement. Since crystals with 3-bromopyruvate were also obtained without glyoxylate, the interactions responsible for the loop closure can be narrowed down to the common carboxylate that binds in the pocket formed by residues His 193, Asn 313, Ser 315, Ser 317, and Thr 347 in both the complexes. With the exception of residue Thr 347, which is positioned identically in both bound and unbound states, all of these residues undergo significant movements upon binding. While His 193 is located on the flexible active site loop, residues Asn 313, Ser 315 and Ser 317 are located at the C-terminal end of strand β14 and undergo a 1˜2 Å shift upon binding.

5. The Catalytic Mechanism of ICL. ICL catalyzes the reversible lysis of a C-C bond of isocitrate to form glyoxylate and succinate. The crystal structures confirm observations that for the reverse reaction, ICL follows a sequential mechanism in which glyoxylate binds first to the enzyme followed by succinate to form a ternary complex (Hoyt et al., 1988). This is based on the observations that glyoxylate is buried deeper in the active site than succinate and that loop closure requires succinate binding. The ICL reaction mechanism involves Claisen condensation via the formation of an enolic intermediate (Kyte, 1995). The key step in the reaction is the deprotonation of the α-proton of a carboxylate of succinate by a base to form 4,4-dihydroxy-3-butenoate. The location of Cys 191 and the ability of its thiol to alkylate with 3-bromopyruvate suggest that it acts as the base and carries out the nucleophilic abstraction of the cc proton from the C2 position of the succinate.

Extending the analogy of thiol proteases to ICL, mutagenesis studies on the enzyme from Escherichia coli led to the proposal that the deprotonation of the active site Cys 191 is carried out by a neighboring His residue (His 193 in M. tuberculosis ICL) (Diehl and McFadden, 1994). In the crystal structures of inhibitor bound ICL, side chains of these residues are located ˜5 Å apart, suggesting that direct proton exchange may not occur between them. A general acid, probably Glu 295, protonates the carboxylate adjacent to the bond formed. The negative charge on the aldehyde oxygen of glyoxylate is stabilized by interactions with an oxyanion hole formed by Mg²⁺ ion, Arg 228 and His 180. While it seems likely that the groups involved in isocitrate lysis are the same, a complete understanding of the C-C cleavage will require the structure of the ICL-isocitrate complex.

The data of the present invention provide two observations that are key to the development of antimicrobial agents. First, the structures of the inhibitor complexes of the invention provide meaningful guidance for the development of drugs targeting ICL for treatment of infections in chronic stages of tuberculosis. The inventors envision that drugs which target ICL will significantly reduce the duration of chemotherapy and act in synergy with the current frontline drugs, isoniazid or rifampicin, that appear to preferentially target rapidly growing organisms. Information from the known substrate analogs can now be used in conjunction with the data and templates provided by the present invention, to build specificity and thereby generate new and more effective inhibitors of ICL.

Second, the present invention is a prime illustration of the requirement for a greater appreciation of the differences between in vivo and in vitro microbial metabolism in designing drug screens effective against intracellular pathogens. The target identified and characterized in these current studies, ICL, represents an enzyme that has been reported in many other microbial pathogens (Mycobacterium spp (Kannan et al., 1985), Pseudomonas (Rao and McFadden, 1965), Salmonella (Wilson and Maloy, 1987), Yersinia (Moncla et al., 1983) and Leishmania (Simon et al., 1978)) that show persistence. This allows the present invention to have application in the development of inhibitors useful for the treatment of persistent infection by pathogens other than M. tuberculosis (Example 4).

### EXAMPLE 4

### Inhibition of the Glyoxylate Shunt in Other Pathogens

In light of the data in the preceding examples, the inventors realized that because the glyoxylate shunt, as exemplified by the isocitrate lyase and malate synthase enzymes, is present in many other microbial pathogens, the present invention provides a unified approach for the development of agents to treat a range of persistence infections.

For example, the enzymes of glyoxylate shunt are also present in M. flavescens, M. vaccae, M. smegmatis and Mycobacteria strain w (M.w.), the latter of which is important as it has a close antigenic resemblance to M. leprae (Kannan et al., 1985), and in Pseudomonas (Rao and McFadden, 1965). Enzymes of the glyoxylate shunt are also present in Salmonella, such as S. typhimurium (Wilson and Maloy, 1987); Yersinia, such as Y. pestis (Moncla et al., 1983); and in Leishmania, including L. brasiliensis, L. donovani, L. mexicana, L. tarentolae and L. tropica (Simon et al., 1978), each of which show persistence. All such organisms and the diseases that result from infection may thus be combated by the present invention, which now provides realistic means to develop inhibitors for use in the treatment of persistent infection by many pathogens of worldwide significance.

Indeed, the glyoxylate shunt is required for fungal virulence, e.g., in Candida albicans and other fungal pathogens. C. albicans, a normal component of the mammalian gastrointestinal flora, is responsible for many fungal infections in immunosuppressed patients, such as patients undergoing treatment for HIV infection and AIDS. In common with M. tuberculosis, Candida and other organisms that are normally phagocytosed by macrophages and/or neutrophils are now amenable to attack based upon inhibiting the glyoxylate shunt.

On ingestion by mammalian macrophages, genes of the glyoxylate shunt are induced in fungi such as Candida and Saccharomyces cerevisiae. Recent data confirms the value of the present invention by additionally showing that phagocytosis in C. albicans up-regulates the principal enzymes of the glyoxylate shunt, isocitrate lyase (ICL1) and malate synthase (MLS1) (Lorenz and Fink, 2001). C. albicans mutants lacking ICL1 are markedly less virulent in mice than the wild type (Lorenz and Fink, 2001). These findings in fungi complement the data from the present inventors showing that isocitrate lyase is both upregulated and required for the virulence of M. tuberculosis (Example 3 in particular). The results thus provide an additional practical demonstration supporting the inventors' concept that the glyoxylate shunt is of wide-ranging significance to microbial pathogenesis and that the enzymes of the glyoxylate shunt are ideal targets for drug development.

The related, but non-pathogenic yeast S. cerevisiae is typically used in the art in studies of host-pathogen interactions relevant to C. albicans. In vitro, cultured mammalian macrophages readily ingest both S. cerevisiae and C. albicans cells. A population of S. cerevisiae highly enriched for phagocytosed cells was isolated and subjected to whole-genome microarray analysis using oligonucleotide-based arrays. Three hours after initiating the co-culture, most of the phagocytosed cells were alive and transcriptional profiling of these cells revealed the response of fungal cells to phagocytosis (Lorenz and Fink, 2001).

Eleven of the fifteen most highly induced S. cerevisiae genes after phagocytosis encoded proteins related to the glyoxylate shunt (Lorenz and Fink, 2001), through which two-carbon compounds are assimilated into the tricarboxylic acid (TCA) cycle. Three of the five glyoxylate shunt enzymes are on this list (isocitrate lyase, ICL1; malate synthase, MLS1; and malate dehydrogenase, MDH2), and a fourth (citrate synthase, CIT2) is also strongly induced (4.9-fold, ranking 24^{th}). Several genes functionally related to glyoxylate shunt are also induced, including acetyl coenzyme A (acetyl-CoA) synthase (ACS1); YDR384c, a homologue of the Yarrowia lipolytica glyoxylate pathway regulator (GPR1); several transporters and acetyltransferases, which are used to traffic intermediates of the glyoxylate shunt and fatty-acid degradation between organelles (CRC1, ACR1, YAT1 and YER024w); and fructose-1,6-biosphosphatase (FBP1), the central regulatory point in gluconeogenesis, of relevance as the production of glucose is the principal function of the glyoxylate shunt (Lorenz and Fink, 2001).

Although the glyoxylate shunt and TCA share common reactions, it is only the isozymes specialized for the glyoxylate shunt that are induced (Lorenz and Fink, 2001). The cytosolic isozyme of MDH2, which preferentially functions in the glyoxylate shunt, is induced 15.6-fold. By contrast, the mitochondrial (MDHl) and peroxisomal (MDH3) forms are not induced. Out of the three citrate synthase isoforms, only the glyoxylate shunt-specific CIT2 is induced (Lorenz and Fink, 2001). In control array studies, expression of glyoxylate shunt enzymes were not changed significantly in response to conditioned media, oxidative stress, or contact with heat-killed macrophages. Thus, phagocytosis specifically upregulates the glyoxylate shunt and its accessory proteins in fungi, as envisioned by the present inventors.

The C. albicans genes for isocitrate lyase (ICL1) and malate synthase (MLS1), the only enzymes whose activity is both specific and limited to the glyoxylate shunt, share significant homology with proteins from fungi, plants and bacteria but, notably, not mammals, which do not have the glyoxylate shunt. Northern analysis of RNA from both S. cerevisiae and C. albicans cells grown in the presence of macrophages showed that in both organisms the ICL1 or MLS1 genes are significantly induced by macrophage contact when compared with cells grown in media alone (Lorenz and Fink, 2001). Thus, the induction of the glyoxylate enzymes is a conserved response to phagocytosis in these two yeasts.

In mutant strains of both S. cerevisiae and C. albicans that lacked ICL1, the icl1 mutant strains fail to use acetate or ethanol. In C. albicans, both the heterozygous strain (≈icl1/ICL1) and a homozygous mutant in which ICL1 has been re-introduced (≈icl1/≈ICL1+ICL1) grow as well as a wild-type strain on acetate media (Lorenz and Fink, 2001). The growth rate of the C. albicans ≈icl1/≈ICL1 strain was not significantly different from the parent strain on rich (YP-Dextrose) media, nor was this strain any more sensitive to a variety of in vivo stresses, including salt, heat shock, ethanol (assayed on glucose media), or oxidative stress. This is in accordance with the inventors' data of the earlier examples.

In testing the virulence of these C. albicans strains in a mouse model of systemic candidiasis, mice injected with wild-type C. albicans strain SC5314 succumbed rapidly to the infection (median survival of 3 days), whereas mice injected with two independently constructed ≈icl1/≈icl1 strains survived longer (Lorenz and Fink, 2001). At day 28, 7 out of 10 of the animals injected with one strain (MCL7) remained alive, as did 6 out of 10 of an independent homozygous mutant (MLC8). Infection with the heterozygotic (≈icl1/ICL1) resulted in an intermediate mortality (median of 8 days). These results therefore confirm the inventors' data that isocitrate lyase is not only induced by macrophage phagocytosis, but is also essential for full virulence in important pathogens, and provide actual data for a fungal pathogen.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods, and in the steps or in the sequence of steps of the methods described herein, without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Abeysinghe, Baker, Rice, Rodgers, Stillman, Ko, McFadden, Nimmo, J. Mol. Biol., 220(1):13-6, 1991.

Bartlett, In Molecular Recognition in Chemical and Biological Problems, Special Pub., Royal Chem. Soc., 78:182-196, 1989.

Bennett, Schlunegger, Eisenberg, Protein Sci., 4:2455-2468, 1995.

Blundel & Johnson, Methods in Enzymology, vols. 114 & 115, H. W. Wyckoff et al., Eds., Academic Press, 1985.

Bohm, J. ComR. Aid. Molec. Design, 6:61-78, 1992.

Britton et al., Structure Fold. Des., 8, 349-362, 2000.

Brunger, et al. Acta Crystallogr. D, 54:905-921, 1998.

Collaborative Computational Project, Number 4, Acta Crystallogr. D, 50:760-763, 1994.

Cohen et al., J. Med. Chem., 33:883-894, 1990.

Diehl and McFadden, J. Bacteriol., 176:927-931, 1994.

Farewell, Diez, DiRusso, Nystrom, J. Bacteriol., 178:6443-6450, 1996.

Goodford, J. Med. Chem., 28:849-857, 1985.

Goodsell & Olsen, Proteins: Structure. Function, and Genetics, 8:195-202, 1990.

Graham and Clark-Curtiss, Proc. Natl. Acad. Sci. U.S.A, 96:11554-11559, 1999.

Hendrickson, Horton, LeMaster, EMBO J., 9:1665-1672, 1990.

Hoener zu Bentrup, Miczak, Swenson, Russell, J. Bacteriol., 181:7161-7167, 1999.

Hoyt, Robertson, Berlyn, Reeves, Biochim. Biophys. Acta, 966:30-35, 1988.

Huang, Li, Jia, Dunaway-Mariano, Herzberg, Structure Fold. Des., 7:539-548, 1999.

Jones, Zou, Kjelgaard, Cowan, Acta Crystallogr. A, 47:110-119, 1991.

Kannan et al., Indian J. Lepr., 57:542-548, 1985.

Ko and McFadden, Arch. Biochem. Biophys., 278:373-380, 1990.

Kuntz, J. Mol. Biol., 161:269-288, 1982.

Kyte, In: Mechanism in Protein Chemistry, Garland, New York, 377-378, 1995.

Lattman, in Methods in Enzymology, 115, pp. 55-77 (1985).

Lorenz and Fink, Nature, 412:83-86, 2001.

Laskowski, MacArthur, Moss, Thornton, J. Appl. Crystallogr., 26:283-291, 1993.

Martin, J. Med Chem., 35:2145-2154, 1992.

Matsuoka and McFadden, J. Bacteriol., 170:4528-4536, 1988.

McKinney et al., Nature, 406:735-738, 2000.

McKinney, Jacobs, Bloom, In: Emerging Infections (eds. Krause, R., Gallin, J. I. and Fauci, A. S.) Academic Press, New York, 51-146, 1998.

Miranker & Karplus, Proteins: Structure, Function and Genetics, 11:29-34, 1991.

Mitchison, J. R. Coll. Physicians Lond., 14:91-95, 98-99, 1980

Moncla, Hillier, Charnetzky, J. Bacteriol., 153:340-344, 1983.

Navaza, Acta Crystallogr. A, 50: 157-163, 1994.

Navia & Murcko, Current Opinions in Structural Biology, 2:202-210, 1992.

Nishibata & Itai, Tetrahedron, 47:8985, 1991.

Otwinowski and Minor, Methods Enzymol., 276:307-326, 1997.

Parrish, Dick, Bishai, Trends Microbiol., 6:107-112, 1998.

Rao and McFadden, Arch. Biochem. Biophys., 112:294-303, 1965.

Rossmann, Ed., Int. Sci. Rev. Ser., No. 13, Gordon & Breach, New York, 1972.

Schaible, Sturgill-Koszycki, Schlesinger, Russell, J. Immunol., 160:1290-1296, 1998.

Schloss and Cleland, Biochemistry, 21:4420-4427, 1982.

Segel, I. H., Enzvme Kinetics, J. Wiley & Sons, 1975.

Segal, In: The Mycobacteria: A Sourcebook (eds. Kubica, G. P. and Wayne, L. G.) Dekker, New York, 547-573, 1984.

Seshadri et al., Indian J. Biochem. Biophys., 13:95-96, 1976.

Simon, Martin, Mukkada, J. Bacteriol., 135:895-899, 1978.

Spector et al., Microbiol 145:15-31, 1999.

Suryanarayana Murthy, Sirsi, Ramakrishnan, Amer. Rev. Resp. Dis. 108:689-690, 1973.

Terwilliger and Berendzen, Acta Crystallogr. D, 55:849-861, 1999.

Vanni, Giachetti, Pinzuati, McFadden, Comp. Biochem. Physiol., 95B:431-458, 1990.

Wayne & Lin, Infect Immun 37:1042-1049, 1982.

Wheeler and Ratledge, J. Gen. Microbiol., 134:2111-2121, 1988.

Wilson and Maloy, 169:3029-3034, 1987.

What is claimed is:

1. A crystallized M. tuberculosis isocitrate lyase enzyme (ICL) characterized as:
(a) a crystallized apo-ICL that has the crystallographic data of Table 1;
(b) a crystallized apo-ICL that has the crystallographic coordinates of FIG. 5, as deposited in the Protein Data Bank under accession code lF61;
(c) a crystallized ICL in complex with the inhibitor 3-brompyruvate and having the crystallographic coordinates of FIG. 6, as deposited in the Protein Data Bank under accession code 1F8M; or
(d) a crystallized ICL in complex with the inhibitor 3-nitropropionate and having the crystallographic coordinates of FIG. 7, as deposited in the Protein Data Bank under accession code 1F8I.

2. The crystallized ICL of claim 1, wherein said crystallized ICL is a crystallized apo-ICL that has the crystallographic data of Table 1.

3. The crystallized ICL of claim 1, wherein said crystallized ICL is a crystallized apo-ICL that has the crystallographic coordinates of FIG. 5, as deposited in the Protein Data Bank under accession code 1F61.

4. The crystallized ICL of claim 1, wherein said crystallized ICL is a crystallized ICL in complex with the inhibitor 3-brompyruvate and having the crystallographic coordinates of FIG. 6, as deposited in the Protein Data Bank under accession code 1F8M.

5. The crystallized ICL of claim 1, wherein said crystallized ICL is a crystallized ICL in complex with the inhibitor 3-nitropropionate and having the crystallographic coordinates of FIG. 7, as deposited in the Protein Data Bank under accession code 1F8I.

6. A computer-readable data storage medium comprising a data storage material encoded with computer-readable data, wherein said data comprises:
(a) the structure coordinates of apo-ICL according to FIG. 5, as deposited in the Protein Data Bank under accession code 1F61;
(b) the structure coordinates of the ICL-3-brompyruvate complex according to FIG. 6, as deposited in the Protein Data Bank under accession code 1F8M;
(c) the structure coordinates of the ICL-3-nitropropionate complex according to FIG. 7, as deposited in the Protein Data Bank under accession code 1F8I; or
(d) the structure coordinates of ICL amino acids His 193, Asn 313, Ser 315 and Ser 317 according to FIG. 5, FIG. 6 or FIG. 7.

7. The data storage medium of claim 6, wherein said computer-readable data comprises:
(a) the structure coordinates of apo-ICL according to FIG. 5, as deposited in the Protein Data Bank under accession code 1F61;
(b) the structure coordinates of the ICL-3-brompyruvate complex according to FIG. 6, as deposited in the Protein Data Bank under accession code 1F8M; or
(c) the structure coordinates of the ICL-3-nitropropionate complex according to FIG. 7, as deposited in the Protein Data Bank under accession code 1F8I.

8. The data storage medium of claim 6, wherein said computer-readable data comprises the structure coordinates of ICL amino acids His 193, Asn 313, Ser 315 and Ser 317 according to FIG. 5, FIG. 6 or FIG. 7.

9. A computer-readable data storage medium comprising a data storage material encoded with computer-readable data, wherein said data comprises the structure coordinates of ICL amino acids His 193, Asn 313, Ser 315 and Ser 317 according to FIG. 5, FIG. 6 or FIG. 7; wherein amino acids His 193, Asn 313, Ser 315 and Ser 317 mediate closure of the active site loop upon binding of an inhibitor to ICL.

10. A computer for producing a three-dimensional representation of:
(a) a molecule or molecular complex comprising the structure coordinates of FIG. 5 (Protein Data Bank accession code 1F61), FIG. 6 (Protein Data Bank accession code 1F8M) or FIG. 7 (Protein Data Bank accession code 1F8I); or
(b) a homologue of said molecule or molecular complex, wherein said homologue comprises structure coordinates that have a root mean square deviation from the backbone atoms of the amino acids of the structure coordinates of FIG. 5, FIG. 6 or FIG. 7 of not more than 1.5 angstroms; wherein said computer comprises:
(i) a computer-readable data storage medium comprising a data storage material encoded with computer-readable data, wherein said data comprises the structure coordinates of FIG. 5, FIG. 6 or FIG. 7;
(ii) a working memory for storing instructions for processing said computer-readable data;
(iii) a central-processing unit coupled to said working memory and to said computer-readable data storage medium for processing said computer-machine readable data into said three-dimensional representation; and
(iv) a display coupled to said central-processing unit for displaying said three-dimensional representation.

11. A computer for producing a three-dimensional representation of:
(a) a molecule or molecular complex that comprises an active site loop defined by the structure coordinates of ICL amino acids His 193, Asn 313, Ser 315 and Ser 317 according to FIG. 5, FIG. 6 or FIG. 7; wherein amino acids His 193, Asn 313, Ser 315 and Ser 317 mediate closure of the active site loop upon binding of an inhibitor to ICL; or
(b) a homologue of said molecule or molecular complex, wherein said homologue comprises an active site loop that has a root mean square deviation from the backbone atoms of said amino acids of not more than 1.5 angstroms, wherein said computer comprises:
(i) a computer-readable data storage medium comprising a data storage material encoded with computer-readable data, wherein said data comprises the structure coordinates of ICL amino acids His 193, Asn 313, Ser 315 and Ser 317 according to FIG. 5, FIG. 6 or FIG. 7;
(ii) a working memory for storing instructions for processing said computer-readable data;
(iii) a central-processing unit coupled to said working memory and to said computer-readable data storage medium for processing said computer-machine readable data into said three-dimensional representation; and
(iv) a display coupled to said central-processing unit for displaying said three-dimensional representation.

12. A computer for determining at least a portion of the structure coordinates corresponding to X-ray diffraction data obtained from a molecule or molecular complex, wherein said computer comprises:
(a) a computer-readable data storage medium comprising a data storage material encoded with machine-readable data, wherein said data comprises at least a portion of the structural coordinates of apo-ICL according to FIG. 5 (Protein Data Bank accession code 1F61), ICL-3-brompyruvate complex according to FIG. 6 (Protein Data Bank accession code 1F8M) or ICL-3-nitropropionate complex according to FIG. 7 (Protein Data Bank accession code 1F8I);
(b) a computer-readable data storage medium comprising a data storage material encoded with computer-readable data, wherein said data comprises X-ray diffraction data obtained from said molecule or molecular complex;
(c) a working memory for storing instructions for processing said computer-readable data of (a) and (b);
(d) a central-processing unit coupled to said working memory and to said computer-readable data storage medium of (a) and (b) for performing a Fourier transform of the machine readable data of (a) and for processing said computer-readable data of (b) into structure coordinates; and
(e) a display coupled to said central-processing unit for displaying said structure coordinates of said molecule or molecular complex.

13. The computer of claim 12, wherein said computer comprises a computer-readable data storage medium comprising a data storage material encoded with machine-readable data, wherein said data comprises the structural coordinates of apo-ICL according to FIG. 5 (1F61), ICL-3-brompyruvate complex according to FIG. 6 (1F8M) or ICL-3-nitropropionate complex according to FIG. 7 (1F8I).

14. The computer of claim 12, wherein said molecule or molecular complex comprises a polypeptide having isocitrate lyase enzyme activity.

15. A method for evaluating the potential of a candidate compound to associate with:
(a) a molecule or molecular complex that comprises an active site loop defined by the structure coordinates of ICL amino acids His 193, Asn 313, Ser 315 and Ser 317 according to FIG. 5, FIG. 6 or FIG. 7; or
(b) a homologue of said molecule or molecular complex, wherein said homologue comprises an active site loop that has a root mean square deviation from the backbone atoms of said amino acids of not more than 1.5 angstroms; wherein said method comprises the steps of:
(i) employing computational means to perform a fitting operation between said candidate compound and an active site loop defined by the structure coordinates of ICL amino acids His 193, Asn 313, Ser 315 and Ser 317 according to FIG. 5, FIG. 6 or FIG. 7 within a root mean square deviation from the backbone atoms of said amino acids of not more than 1.5 angstroms; and
(ii) analyzing the results of said fitting operation to quantify the association between said candidate compound and said active site loop.

16. The method of claim 15, wherein said method evaluates the potential of said candidate compound to associate with:
(a) a molecule or molecular complex defined by structure coordinates of all amino acids of ICL, wherein said structure coordinates are selected from the group consisting of apo-ICL according to FIG. 5 (1F61), ICL-3-brompyruvate complex according to FIG. 6 (1F8M) and ICL-3-nitropropionate complex according to FIG. 7 (1F8I); or
(b) a homologue of said molecule or molecular complex having a root mean square deviation from the backbone atoms of said amino acids of not more than 1.5 angstroms.

17. A method for identifying an inhibitor of a microbial isocitrate lyase enzyme (ICL), comprising:
(a) defining the catalytic active site of ICL from the atomic coordinates selected from the group consisting of the atomic coordinates for the apo-ICL of FIG. 5 (Protein Data Bank accession code 1F61), the atomic coordinates for the ICL-3-brompyruvate complex of FIG. 6 (Protein Data Bank accession code 1F8M) and the atomic coordinates for the ICL-3-nitropropionate complex of FIG. 7 (Protein Data Bank accession code 1F 8I); and
(b) identifying a non-native substrate compound that fits said active site and thereby inhibits said microbial isocitrate lyase enzyme.

18. A method for identifying an inhibitor of a microbial ICL, comprising:
(a) obtaining atomic coordinates of ICL, wherein said atomic coordinates are selected from the group consisting of the apo-ICL atomic coordinates of FIG. 5 (Protein Data Bank accession code 1F61), the ICL-3-brompyruvate complex atomic coordinates of FIG. 6 (Protein Data Bank accession code 1F8M) and the ICL-3-nitropropionate complex atomic coordinates of FIG. 7 (Protein Data Bank accession code 1F8I);
(b) defining the catalytic active site of ICL from said atomic coordinates; and
(c) identifying a non-native substrate compound that fits said catalytic active site, wherein a non-native substrate compound that fits said active site is indicative of an inhibitor of a microbial ICL.

19. The method of claim 18, wherein said non-native substrate compound that fits said catalytic active site is identified by selecting a candidate compound and confirming that said candidate compound inhibits said microbial ICL.

20. A method for identifying an inhibitor of a microbial ICL, comprising:
(a) obtaining atomic coordinates of ICL, wherein said atomic coordinates are selected from the group consisting of the apo-ICL atomic coordinates of FIG. 5 (Protein Data Bank accession code 1F61), the ICL-3-brompyruvate complex atomic coordinates of FIG. 6 (Protein Data Bank accession code 1F8M) and the ICL-3-nitropropionate complex atomic coordinates of FIG. 7 (Protein Data Bank accession code 1F8I);
(b) defining the catalytic active site of ICL from said atomic coordinates;
(c) selecting a candidate compound by identifying a non-native substrate compound that fits said catalytic active site; and
(d) contacting said microbial ICL with said candidate compound under conditions effective for ICL activity, wherein a candidate compound that inhibits the activity of said microbial ICL is confirmed as an inhibitor of said microbial ICL.

21. The method of claim 20, wherein said candidate compound is selected from consideration of a database of compounds.

22. The method of claim 20, wherein said candidate compound is selected by de novo design.

23. The method of claim 20, wherein said candidate compound is selected by design starting from a known inhibitor.

24. The method of claim 20, wherein said candidate compound is selected by identifying a compound intended to interact with at least one of ICL amino acids His 193, Asn 313, Ser 315 or Ser 317 according to FIG. 5, FIG. 6 or FIG. 7.

25. The method of claim 24, wherein a candidate compound intended to interact with at least one of said ICL amino acids His 193, Asn 313, Ser 315 or Ser 317 is selected by employing computational means to perform a fitting operation between said candidate compound and a binding pocket defined by said amino acids within a root mean square deviation from the backbone atoms of said amino acids of not more than 1.5 angstroms.

26. The method of claim 20, wherein the inhibitor so identified inhibits a mycobacterial ICL.

27. The method of claim 26, wherein the inhibitor so identified inhibits M. tuberculosis ICL.

28. The method of claim 20, wherein the inhibitor so identified inhibits a fungal ICL.

29. The method of claim 20, wherein the inhibitor so identified inhibits said microbial ICL by changing the structure of said active site from the open conformation to the closed conformation.

30. The method of claim 20, wherein the inhibitor so identified is a competitive inhibitor.

31. The method of claim 20, wherein the inhibitor so identified is a non-competitive or uncompetitive inhibitor.

32. The method of claim 20, further comprising purifying or synthesizing the inhibitor so identified.

33. The method of claim 20, further comprising formulating the inhibitor so identified in a pharmaceutically acceptable formulation.

34. The method of claim 33, wherein said pharmaceutically acceptable formulation further comprises at least a second antimicrobial agent.

35. A method for identifying a potential agonist or antagonist of a molecule comprising a microbial ICL-like active site loop, said method comprising the steps of:
(a) using the atomic coordinates of ICL amino acids His 193, Asn 313, Ser 315 and Ser 317 according to FIG. 5, FIG. 6 or FIG. 7 within a root mean square deviation from the backbone atoms of said amino acids of not more than 1.5 angstroms, to generate a three-dimensional structure of molecule comprising a microbial ICL-like active site loop;
(b) employing said three-dimensional structure to design or select said potential agonist or antagonist;
(c) purifying or synthesizing said potential agonist or antagonist; and
(d) contacting said potential agonist or antagonist with said molecule to determine the ability of said potential agonist or antagonist to interact with said molecule.

36. The method of claim 35, wherein step (a) comprises using the atomic coordinates of all the amino acids of ICL according to FIG. 5, FIG. 6 or FIG. 7 within a root mean square deviation from the backbone atoms of said amino acids of not more than 1.5 angstroms.

37. A method for identifying an agent for use in the treatment of chronic tuberculosis, comprising:
(a) obtaining atomic coordinates of M. tuberculosis ICL, wherein said atomic coordinates are selected from the group consisting of the apo-ICL atomic coordinates of FIG. 5 (Protein Data Bank accession code 1F61), the ICL-3-brompyruvate complex atomic coordinates of FIG. 6 (Protein Data Bank accession code 1F8M) and the ICL-3-nitropropionate complex atomic coordinates of FIG. 7 (Protein Data Bank accession code 1F8I);
(b) defining the catalytic active site of ICL from said atomic coordinates;
(c) selecting a candidate compound by identifying a non-native substrate compound that fits said catalytic active site; and
(d) contacting M. tuberculosis ICL with said candidate compound under conditions effective for ICL activity, wherein a candidate compound that inhibits the activity of said M. tuberculosis ICL is indicative of an agent for use in the treatment of chronic tuberculosis.

38. A method for identifying an agent for use in treating or preventing a persistent microbial infection, comprising selecting an enzyme target from the glyoxylate shunt pathway in an intracellular microbial pathogen and identifying a compound that inhibits said enzyme target, thereby identifying an agent for use in treating or preventing a persistent microbial infection.

39. The method of claim 38, wherein said selected enzyme target is isocitrate lyase or malate synthase.

40. The method of claim 38, wherein said selected enzyme target is an enzyme from the glyoxylate shunt pathway in a mycobacterium.

41. The method of claim 38, wherein said selected enzyme target is an enzyme from the glyoxylate shunt pathway in a fungus.

42. The method of claim 38, wherein a compound that inhibits the selected enzyme target is identified by testing the ability of said compound to inhibit the growth of said intracellular microbial pathogen when grown on a carbon source in vitro that mimics the nutrient environment encountered during the persistent phase of infection in vivo.

43. The method of claim 42, wherein a compound that inhibits the selected enzyme target is identified by testing the ability of said compound to differentially inhibit the growth of said intracellular microbial pathogen when grown in vitro on a C₂ carbon source versus a C₆ carbon source.

44. The method of claim 38, wherein a compound that inhibits the selected enzyme target is identified by a method comprising:
(a) testing the ability of said compound to inhibit the activity of the selected enzyme target in a cell-free enzyme activity assay; and
(b) confirming the ability of said compound to significantly inhibit the growth of said intracellular microbial pathogen when grown on acetate in vitro, but to not significantly inhibit the growth of said intracellular microbial pathogen when grown on glucose in vitro, thereby favoring the selection of an inhibitor that preferentially inhibits an enzyme of the glyoxylate shunt pathway operative in persistent infection of inflammatory macrophages in vivo.

45. The method of claim 38, wherein a compound that inhibits the selected enzyme target is identified by selecting a candidate inhibitor based upon the crystal structure of said selected enzyme target.
